(19) United States
(12) Patent Application Publication
(10) Pub. No.: US 2024/0217968 A1
(43) Pub. Date: Jul. 4, 2024

(54) PHARMACEUTICAL COMPOUND

(71) Applicant: DUKE STREET BIO LIMITED, London (GB)

(72) Inventors: Phillip Martin Cowley, London (GB); Barry Edward McGuiness, London (GB); Alan Wise, London (GB); Allan Michael Jordan, Nottingham (GB); Kamaldeep Kaur Chohan, Nottingham (GB); Morgan Jouanneau, Nottingham (GB)

(21) Appl. No.: 18/284,682

(22) PCT Filed: Mar. 30, 2022
(86) PCT No.: PCT/EP2022/058490

§ 371 Date: Sep. 28, 2023

(30) Foreign Application Priority Data
Mar. 31, 2021 (GB) 2104664.4
Oct. 6, 2021 (GB) 2114315.1

(51) Int. Cl. C07D 471/04; A61K 31/496; A61K 31/502; A61K 31/5025; A61K 31/506; A61K 31/517; A61K 31/519; A61K 31/53; A61K 31/551; A61K 45/06; A61P 35/00; A61P 37/04; C07D 237/32; C07D 239/88; C07D 239/96; C07D 401/10; C07D 401/12; C07D 401/14; C07D 403/12; C07D 403/14; C07D 413/14; C07D 417/12; C07D 417/14; C07D 487/04; C07D 495/04; C07D 498/04

(52) U.S. Cl. CPC C07D 471/04; A61K 31/496; A61K 31/502; A61K 31/5025; A61K 31/506; A61K 31/517; A61K 31/519; A61K 31/53; A61K 31/551; A61K 45/06; A61P 35/00; A61P 37/04; C07D 237/32; C07D 239/88; C07D 239/96; C07D 401/10; C07D 401/12; C07D 401/14; C07D 403/12; C07D 403/14; C07D 413/14; C07D 417/12; C07D 417/14; C07D 487/04; C07D 495/04; C07D 498/04

(57) ABSTRACT

A compound comprising the following formula:

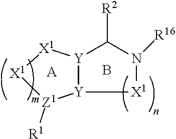

wherein each X¹ is independently selected from C, N, O and S; each Y is independently selected from C and N; Z¹ is independently selected from C and N; each X¹ may independently be unsubstituted, or may independently be substituted with H or a substituted or unsubstituted organic group; each Y may independently be unsubstituted, or may independently be substituted with H or a substituted or unsubstituted organic group; m may be 1, 2, 3 or 4; n may be 1, 2, or 3; the bonds between all of the atoms in ring A may independently be single bonds or double bonds; the bonds between all of the atoms in ring B may independently be single bonds or double bonds; and wherein R¹ may be attached to Z¹ by a single bond or a double bond.

The present invention relates to PARP7 inhibitor compounds, and in particular to PARP7 inhibitor compounds for use in medicine. The inhibitors of the invention may be used in pharmaceutical compositions, and in particular pharmaceutical compositions for treating a cancer, an infectious disease, a central nervous system disease or disorder, a pain condition and other diseases, conditions and disorders. The invention also relates to methods of manufacture of such inhibitors, and methods of treatment using such inhibitors.

### BACKGROUND TO THE INVENTION

Monoclonal antibody-based therapeutics targeting immune checkpoints, most notably the PDL1-PD1 axis, are transforming approaches to the treatment of cancer. These agents have been demonstrated to elicit complete and durable regressions of metastatic disease, most notably in the setting of malignant melanoma. PDL1 expressed by tumour (and other) cells delivers an inhibitory signal via ligation of PD1 on T-cells. Blocking this interaction with antibodies targeting PD1 or PDL1 results in T-cell reactivation, recognition of tumour cell neoantigens and CD8+ve T-cell-mediated tumour cell killing (Hashem O. et al. PD-1 and PD-L1 Checkpoint Signalling Inhibition for Cancer Immunotherapy: Mechanism, Combinations, and Clinical Outcome. Front Pharmacol. 8: 561, (2017)). Despite these developments the fact remains that tumour responses are only observed in a minority of cancer patients. Furthermore, in many patients that do respond responses are not durable. There is an urgent need to identify and develop complementary therapies that will broaden the population for whom immunomodulatory therapy delivers benefit.

Immune checkpoint inhibitors (ICIs) such as anti-PD1 and anti-PDL1 act by relieving checkpoint restraints on anti-tumour T cell responses. They work best against immunogenic, T-cell inflamed or hot tumours. In contrast, ICIs are poorly efficient in cold tumour microenvironments (TMEs) that are largely devoid of T cells and infiltrated by immunosuppressive cells. In hot TMEs, increased expression of type I interferons (IFN-I) and IFN-stimulated genes (ISGs), such as T-cell attracting chemokines, contribute to potent anti-tumour responses. One emerging therapeutic strategy to transform cold tumours into hot exploits the use of pattern recognition receptor (PRR) agonists. Indeed, combinations of ICIs with agonists of RIG-I Helicase, Toll-like receptor 9 (TLR9) or stimulator of interferon genes (STING) have now reached clinical evaluation.

The innate immune system provides a first line of host defence and plays a crucial role in initiating and driving the development of adaptive immune responses. The cytosolic DNA sensor cyclic GMP-AMP synthase (cGAS) can be activated by double stranded DNA arising from the genomes of invading pathogens and also by aberrant cytosolic levels of host DNA that are generated in tumour cells (Chen Q et al. Regulation and function of the cGAS-STING pathway of cytosolic DNA sensing. Nat Immunol. 17: 1142-9, (2016)). Activation of cGAS leads to the generation of cyclic guanosine monophosphate-adenosine monophosphate (cGAMP) which induces dimerization of Stimulator of interferon genes (STING). STING subsequently translocates from the endoplasmic reticulum to the Golgi where it recruits and activates TANK-binding kinase 1 (TBK1). TBK1 phosphorylates interferon regulatory transcription factor 3 (IRF3) which drives the production of type I interferons and supports the generation of immunity (Zhu Y et al. STING: a master regulator in the cancer-immunity cycle. Mol Cancer 18: 152 (2019)). As such, activation of the STING pathway has become of increasing interest to the cancer drug discovery community as a potential strategy to boost the development of adaptive immune responses to tumour cell neoantigens (Sivick K. E. et al. Magnitude of Therapeutic STING Activation Determines CD8+ T Cell-Mediated Anti-tumor Immunity. Cell Reports. 25: 3074, (2018)). Cytoplasmic DNA sensing has also been linked to inactivation of cellular proliferation providing an additional potential mechanistic axis that may contribute to control of tumorigenesis (Paludan S. R. et al. DNA-stimulated cell death: implications for host defence, inflammatory diseases and cancer. Nat Rev Immunol. 19: 141-153, (2019)).

Cancer cells can exhibit a chronic Interferon-stimulate gene (ISG) signature triggered by a STING-dependent pathway, which results in a unique primed cancer cell state that is sensitized to respond to aberrant nucleic acid accumulation (Liu H et al. Tumor-derived IFN triggers chronic pathway agonism and sensitivity to ADAR loss. Nat Medicine. 25: 95-102, 2019). It has recently been shown that genomic instability, in the form of unrepaired DNA double-strand breaks or micronuclei disruption can trigger STING-dependent anti-tumour responses. For example, use of chemotherapeutics can lead to higher levels of aberrant DNA in the cytosol which in turn can trigger cancer cell intrinsic STING signalling leading to anti-tumour immunity. Indeed the efficacy of the commonly used chemotherapeutic drug 5-fluorouracil (5-FU) was recently shown to depend on anti-tumor immunity triggered by the activation of cancer-cell intrinsic STING (Tian J et al. 5-Fluorouracil efficacy requires anti-tumor immunity triggered by cancer-cell-intrinsic STING. EMBO J. 40: e106065 (2021). In addition, PARP inhibitor-induced STING pathway activation and anti-tumor immune responses have been demonstrated in multiple tumour models, providing rationale for exploiting combinations of PARP inhibitors with immunotherapies for improved therapeutic efficacy. For example the PARP inhibitor Olaparib was also recently shown to induce synthetic lethal effects in combination with a synthetic cyclic dinucleotide STING agonist in DNA damage repair deficient cancer cells and a BRCA-deficient breast cancer model (Pantelidou C et al. STING agonism enhances anti-tumor immune responses and therapeutic efficacy of PARP inhibition in BRCA-associated breast cancer. bioRxiv (2021). The authors hypothesize that STING agonism can enhance the therapeutic efficacy of PARP inhibitors in BRCA-associated triple-negative breast cancer (TNBC).

Overall, modulation of nucleic acid sensing pathways via multiple mechanisms has been shown to promote anti-tumour efficacy in a variety of cell and animal models thus demonstrating therapeutic potential for augmenting efficacy of immunotherapies and overcoming resistance to immune checkpoint blockade.

Poly-ADP-ribose polymerase 7 (PARP7, TIPARP, ARTD14), a member of the wider PARP enzyme family, modulates protein function by using nicotinamide adenine dinucleotide (NAD+) as a substrate to transfer an ADP-ribose monomer onto specific amino acid acceptor residues of target proteins (Gomez A et al. Characterisation of TCDD-inducible poly-ADP-ribose polymerase (TIPARP/ARTD14) catalytic activity. Biochemical Journal. 475: 3827-3846, (2018)). PARP7 catalyses mono-ADP ribosylation (MARylation) of its target substrates and as such is a member of the mono(ADP-ribosyl) transferase (MART) enzymes, a subclass of the PARP family of enzymes (reviewed in-Challa L. et al. MARTs and MARylation in the Cytosol: Biological Functions, Mechanisms of Action, and Therapeutic Potential. Cells 10, 313 (2021)). PARP7 is a target gene of the Aryl Hydrocarbon Receptor (AHR) which is a ligand-activated transcription factor and member of the basic helix-loop-helix/Per-AHR nuclear translocator (ARNT)-Sim (PAS) protein family which plays a central role in controlling immune responses. Therefore, PARP7 has emerged as a critical regulator of the innate immune response. The PARP7 gene is amplified in a number of cancers, notably those of the upper aerodigestive tract (Vasbinder, M. M. et al. RBN-2397: A First-in-class PAPR7 inhibitor targeting a newly discovered cancer vulnerability in stress-signalling pathways. Cancer Res. 80:16 suppl DDT02-01, (2020)). PARP7 has been reported to ADP ribosylate and inactivate the kinase domain of TBK1 resulting in suppression of a central pathway for interferon production (Yamada T et al. Constitutive aryl hydrocarbon receptor signalling constrains type I interferon-mediated antiviral innate defence. Nature Immunol. 17: 687-694, (2016)). The possibility of using PARP7 inhibitors in cancer therapy, especially in the treatment of lung squamous cell carcinoma, has been described in WO 2016/116602. The discovery of a potent and selective inhibitor of PARP7, RBN-2397 has been recently reported (Vasbinder, M. M. et al. RBN-2397: A First-in-class PAPR7 inhibitor targeting a newly discovered cancer vulnerability in stress-signalling pathways. Cancer Res. 80:16 suppl DDT02-01, (2020); Gozgit J et al. PARP7 negatively regulates the type I interferon response in cancer cells and its inhibition leads to tumour regression. Cancer Res. 80:16 suppl 3405, (2020); Gozgit J et al. PARP7 negatively regulates the type I interferon response in cancer cells and its inhibition triggers antitumor immunity. Cancer Cell 39: 1-13, 2021). RBN-2397 potently inhibited proliferation in cancer cell lines with high baseline expression of interferon stimulated genes and restored type I interferon responses both in vitro and in vivo resulting in tumour regression and establishment of specific anti-tumour immunity in animal models. WO 2019/212937 describes pyridazinone compounds as inhibitors of PARP7 for use in the treatment of cancer. The monocyclic pyridazinone ring is claimed as an essential feature in the interaction with the PARP7 target. These observations provide a rational basis for generating novel agents to inhibit PARP7 and induce therapeutic anti-tumour responses.

There is also an established and growing literature highlighting key roles of PARP7 in other diseases:

### Infectious Diseases

The inactive PARP family member, PARP13, which plays a key role in regulating the antiviral innate immune response, is a major substrate of PARP7 (Rodriguez, K et al. Chemical genetics and proteome-wide site mapping reveal cysteine MARylation by PARP-7 on immune-relevant protein targets. Elife. 10:e60480, (2021)). PARP13 is preferentially MARylated on cysteine residues in its RNA binding zinc finger domain. PARP13 stimulates the interferon response in response to influenza A viral infection via direct activation of the cytosolic nucleic acid sensor RNA helicase RIG-I. This interaction is dependent on the finger domains of PARP13. Hence Cys MARylation of PARP13 by PARP7 could potentially disrupt the interaction between PARP13 and RIG-I thus regulating its antiviral and immune regulatory roles.

In addition, PARP7 promotes influenza A virus infection by ADP-ribosylating TBK1, which inhibits type I IFN (IFN-I) production (Yamada T. et al. Constitutive aryl hydrocarbon receptor signaling constrains type-I-interferon-mediated antiviral innate defense. Nat. Immunol. 17: 687-694, (2016)). The same study found that constitutive AHR signalling negatively regulated the type I interferon (IFN-I) response during infection with various types of virus; therefore revealing the physiological importance of endogenous activation of AHR signalling in shaping the IFN-I-mediated innate response and, further, suggesting that the AHR-PARP7 axis is a potential therapeutic target for controlling antiviral responses.

More recently (Heer C. et al. Coronavirus infection and PARP expression dysregulate the NAD Metabolome: an actionable component of innate immunity. J Biol Chem. 195, 17986-17996 (2020)) it has been shown that SARS-CoV-2 infection strikingly upregulates MARylating PARPs including PARP7. and induces the expression of genes encoding enzymes for salvage NAD synthesis from nicotinamide (NAM) and nicotinamide riboside (NR), while downregulating other NAD biosynthetic pathways. Furthermore, infection of mice with mouse hepatitis virus (MHV), a coronavirus (CoV), stimulated upregulation of downstream effector PARP7 via activation of the AHR. Knockdown of PARP7 reduced viral replication and increased interferon expression, suggesting that PARP7 functions in a proviral manner during MHV infection (Grunewald M. E. et al. Murine Coronavirus Infection Activates the Aryl Hydrocarbon Receptor in an Indoleamine 2,3-Dioxygenase-Independent Manner, Contributing to Cytokine Modulation and Proviral TCDD-Inducible-PARP Expression. J Virology 94: e01743-19 (2020). The AhR is also overexpressed following coronavirus infection, including SARS-CoV-2 and, as it regulates PARP gene expression, the latter is likely to be activated in COVID-19 (Badawy A. Immunotherapy of COVID-19 with poly (ADP-ribose) polymerase inhibitors: starting with nicotinamide. Bioscience Reports. 40: BSR20202856 (2020)). Therefore, given its key role in the innate immune system, PARP7 inhibition could be used to improve the outcome of patients with a wide variety of infectious diseases including those driven by viral infection.

### Central Nervous System Diseases

PARP7 affects neural progenitor cell proliferation and migration, and its loss leads to aberrant organization of the mouse cortex during development (Grimaldi G et al. Loss of Tiparp Results in Aberrant Layering of the Cerebral Cortex. ENeuro 6(6) 0239-19.2019). PARP7 is highly expressed in the brain with increased expression reported in a range of neurological diseases. PARP7 was identified as a highly upregulated protein following trace fear conditioning and in neurologic disorders, such as epilepsy (Dachet et al. Predicting novel histopathological microlesions in human epileptic brain through transcriptional clustering. Brain 138:356-370, (2015)). In an integrated multi-cohort transcriptional meta-analysis of neurodegenerative diseases including Alzheimers Disease, Amyotrophic Lateral Sclerosis, Parkinsons Disease and Huntingdons Disease, PARP7 was shown to be strongly upregulated (Li et al. Integrated multi-cohort transcriptional meta-analysis of neurodegenerative diseases. Acta Neuropathol Commun 2:93 (2014)). The phenotype of the PARP7−/− mice and expression pattern suggests that alterations in PARP7 expression or function could increase susceptibility to a wide range of both developmental and degenerative neurologic diseases and that inhibitors may potentially show beneficial effects in these conditions.

### Nociception

It has recently been reported that STING is a critical regulator of nociception mediated through induction of type I interferon production and subsequent activation of type I interferon receptors on sensory neurons (Donnelly C R et al. STING controls nociception via type I interferon signalling in sensory neurons. Nature. 591: 275-280 (2021)). Mice lacking STING exhibit hypersensitivity to nociceptive stimuli whereas STING activation elicits marked antinociception in mice and non-human primates. PARP7 is a negative regulator of the STING pathway and inhibitors of PARP7 have been shown to activate this pathway. Such inhibitors may have utility as antinociceptive agents and the treatment of chronic pain conditions including cancer-associated pain and peripheral neuropathy.

In addition, there is therapeutic potential for use of a PARP7 inhibitor in canine cancer as notably recent data showed that intratumoral delivery of a STING agonist resulted in clinical responses in canine glioblastoma (Boudreau C E et al. Delivery of STING Agonist Results in Clinical Responses in Canine Glioblastoma. Clin Cancer Res (2021).

Having regard to the above, it is an aim of the present invention to provide PARP7 inhibitors, and in particular PARP7 inhibitors for use in medicine. It is a further aim to provide pharmaceutical compositions comprising such inhibitors, and in particular to provide compounds and pharmaceutical compositions for treating a cancer, an infectious disease, a central nervous system disease or disorder and other diseases, conditions and disorders. It is also an aim to provide methods of synthesis of the compounds.

### SUMMARY OF THE INVENTION

Accordingly, the present invention provides a PARP7 inhibitor compound, which compound comprises the following formula:

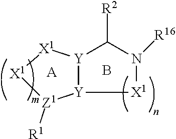

wherein each X¹ may be the same or different and is independently selected from C, N, O and S; each Y may be the same or different and is independently selected from C and N; Z¹ is independently selected from C and N; each X¹ may independently be unsubstituted, or may independently be substituted with H or a substituted or unsubstituted organic group; each Y may independently be unsubstituted, or may independently be substituted with H or a substituted or unsubstituted organic group; Z¹ may independently be further substituted with H or a substituted or unsubstituted organic group; m may be 1, 2, 3 or 4; n may be 1, 2, or 3; the bonds between all of the atoms in ring A may independently be single bonds or double bonds provided that when X¹ is O or S the bonds to that X¹ are single bonds; the bonds between all of the atoms in ring B may independently be single bonds or double bonds provided that when X¹ is O or S the bonds to that X¹ are single bonds;
and wherein R¹ may be attached to Z¹ by a single bond or a double bond and is a substituent of formula:

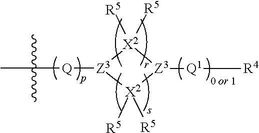

wherein each Q may be the same or different an is independently selected from C, N, O and S; each Q may independently be attached to another Q, or to Z³, by a single bond or a double bond; each Q may independently be unsubstituted, or may independently be substituted by H or a substituted or unsubstituted organic group; two or more Q atoms may form a ring together with their substituents; p is a number from 2 to 8; each Z³ may be the same or different and is independently selected from C and N; each Z³ may independently be further substituted with H or a substituted or unsubstituted organic group; each X² may be the same or different and is independently selected from C, N, O and S; r is a number from 1 to 5; s is independently a number from 1 to 5; wherein Q¹ is selected from C, N, O and S and may be attached to Z³ and R⁴, by a single bond or a double bond and may be unsubstituted, or substituted by H or an organic group; and R⁴ is a substituted or unsubstituted organic group comprising a substituted or unsubstituted carbocyclic or heterocyclic ring; each bond in the ring comprised of Z³ and X² atoms may independently be a double bond or a single bond, provided that when X² is O or S the bonds to that X² are single bonds; each R⁵ may be present or absent depending on the number of bonds to, and the valence of, the X² atom attached to that R⁵; and wherein each R⁵ is independently selected from H or a substituted or unsubstituted organic group;
and wherein R² may be attached to ring B by a single bond or a double bond and is a substituted or unsubstituted organic group;
and wherein R¹⁶ may be present or absent and when present is selected from H, a C₁-C₆ alkyl group or a linear or branched C₁-C₆ halogenated alkyl group.

Typically, if an R⁵ group is a substituent on a C atom, then that R⁵ may be selected from any substituent that R⁷ or R⁸ may be, and if an R⁵ group is a substituent on an N atom, then that R⁵ may be selected from any substituent that R⁶ or R⁹ may be.

In the present invention, both above and in the following, where a substituent is possible, but not depicted in a formula, the number of substituents borne by any atom is the number required to maintain the valency for that atom. For example, in the formula above, the groups X¹ and Y may be unsubstituted or substituted. The substituents have not been shown explicitly in the formula since the number of such substituents (and their presence or absence) will depend on the number of bonds to, and the valence of, the X¹ atom or Y atom comprising a substituent. In general, both above and in the following, where a substituent is possible, but not depicted in a formula, the number of substituents borne by any atom is the number required to maintain the valency for that atom. Similarly, in some cases a substituent has been depicted, but the number of such substituents (and their presence or absence) depends on the number of bonds to, and the valence of, the atom comprising the substituent. In those cases, both above and in the following, the number of substituents borne by the atom is again the number required to maintain the valency for that atom.

In the context of the present invention, maintaining the valency means ensuring that an atom has its normal (typically most common) valency in organic compounds (for example 2 for oxygen and sulphur, 3 for nitrogen and 4 for carbon). Nitrogen atoms may, in some instances, have 4 bonds, but in such cases they are typically positively charged such that the compound may have a counter-ion. Sulphur atoms may, in some instances, have a higher valency such as 6, for example when forming a sulphonyl group. Such compounds are also considered to be part of the invention. When there is a positive charge on a nitrogen, it will be clear that the nitrogen atom still maintains its normal valency of 3. For the avoidance of doubt, where the number of R groups may vary according to the choice of X, Y or Z group, it may vary as follows.

Each R⁵ may be the same or different, provided that for each X²: R⁵ is absent when X² is N and is double bonded to a ring atom; one R⁵ is present when X² is N and is not double bonded to a ring atom; one R⁵ is present when X² is C and is double bonded to a ring atom; and two R⁵ are present when X² is C and is not double bonded to a ring atom.

R¹⁶ is absent when the N to which it is attached in ring B is double bonded to a ring atom; R¹⁶ is present when the N is not double bonded to a ring atom.

Each R¹¹ may be the same or different, provided that for each X⁴: R¹¹ is absent when X⁴ is O or divalent S; R¹¹ is absent when X⁴ is N and is double bonded to an adjacent atom; one R¹¹ is present when X⁴ is N and is not double bonded to an adjacent atom; one R¹¹ is present when X⁴ is C and is double bonded to an adjacent atom; two R¹¹ are present when X⁴ is C and is not double bonded to an adjacent atom; and two R¹¹ are present, each as double bonded O when X⁴ is hexavalent S.

R¹² is absent when the Z⁶ to which it is attached is O or S; R¹² is absent when the Z⁶ is N and is double bonded to a ring atom; R¹² is present when the Z⁶ is N and is not double bonded to a ring atom; R¹² is present when the Z⁶ is C and is double bonded to a ring atom; R¹² is present when the Z⁶ is C and is single bonded to a ring atom and bears a further substituent.

In these compounds, and elsewhere herein, in some embodiments any R group (with the exception of R¹) may form a ring with any other R group on an adjacent and/or proximal atom, although in most embodiments this is not preferred, except where explicitly stated. Thus, in some embodiments the following substituents may together form a ring: R⁵ with another R⁵; R⁵ with R⁴; R⁶ with another R⁶; R⁶ with R⁷; R⁷ with another R⁷; R⁸ with another R⁸; R⁸ with R⁹; R⁹ with another R⁹; R⁶ with R⁸; R⁶ with R¹¹; and R¹¹ with another R¹¹. In the context of the present invention, an adjacent and/or proximal atom may mean another atom directly bonded to an atom (adjacent), or may be two atoms with only a single atom in between (proximal), or may mean two atoms close enough sterically to be capable of forming a ring (proximal). Preferably R groups attached to the same atom do not together form a ring, although this is not excluded.

In the present context the invention includes compounds in which a single R group on an atom, or two R groups on the same atom, form a group which is double bonded to that atom. Accordingly, an R group, or two R groups attached to the same atom, may together form a ═O group, or a ═C(R′)₂ group (wherein each R′ group is the same or different and is H or an organic group, preferably H or a straight or branched C₁-C₆ alkyl group). This is more typical in cases where the R groups are attached to a C atom, such that together they form a C═O group or a C═C(R′)₂ group. Thus is some cases a C ring atom in a ring may comprise a ═O group, as may any X, any Z, and/or and one or more of R², R⁵, R⁷, R⁸, and R¹¹.

In the present context, part of any structure present in brackets may be repeated the number of times given by the numbers next to the brackets (whether regular brackets or square brackets). For example, in the case of (C(R))_{0,1,2} or [C(R)]_{0,1,2} the C—R group may be absent, present once i.e. —C(R)—; or present twice i.e. —C(R)—C(R)—.

Further in the present context, where a structural component is depicted with a wavy line on a bond, that bond is the bond that attaches to another structural component of the compound.

In the context of the present invention, a compound is considered to be a PARP7 inhibitor if its presence is capable of preventing or reducing the ability of immobilised PARP7 to undergo auto-mono-ADP ribosylation (AutoMARylation) following incubation with biotinylated-NAD⁺ as compared to the same process in its absence. Typically, the compound is considered to be a PARP7 inhibitor if it has an IC₅₀<10 μM in a suitable assay. A suitable assay may be conducted using 10-30 nM PARP7 (amino acids 456-657), 2 μM biotin-NAD assay solution in 20 mM HEPES (pH 7.5), 100 mM NaCl, 2 mM DTT, 0.1% BSA (w/v), 0.02% Tween (v/v) assay buffer. MARylation may take place for 2-3 h at room temperature and may be detected using a dissociation-enhanced lanthanide fluorescence immunoassay (DELFIA) readout. This assay format has been recently utilised for screening for modulators of PARP7 and other MonoPARP enzymes (Wigle T. et al. Forced Self-Modification Assays as a Strategy to Screen MonoPARP Enzymes. SLAS Discovery. 25; 241-252, (2020)). A particularly suitable assay is described in the Examples below.

In all of the embodiments of this invention (both above and below herein), the substituents (each of the R groups) are not especially limited, provided that they do not prevent the PARP7 inhibitory function from occurring. In all of the embodiments mentioned in connection with this invention, both above and in the following, the substituents are selected from H and an organic group. Thus, both above and in the following, the terms ‘substituent’ and ‘organic group’ are not especially limited and may be any functional group or any atom, especially any functional group or atom common in organic chemistry. Thus, ‘substituent’ and ‘organic group’ may have any of the following meanings.

The organic group may comprise any one or more atoms from any of groups IIIA, IVA, VA, VIA or VIIA of the Periodic Table, such as a B, Si, N, P, O, or S atom (e.g. OH, OR, NH₂, NHR, NR₂, SH, SR, SO₂R, SO₃H, PO₄H₂) or a halogen atom (e.g. F, Cl, Br or I) where R is a linear or branched lower hydrocarbon (1-6 C atoms) or a linear or branched higher hydrocarbon (7 C atoms or more, e.g. 7-40 C atoms).

The organic group preferably comprises a hydrocarbon group. The hydrocarbon group may comprise a straight chain, a branched chain or a cyclic group. Independently, the hydrocarbon group may comprise an aliphatic or an aromatic group. Also independently, the hydrocarbon group may comprise a saturated or unsaturated group.

When the hydrocarbon comprises an unsaturated group, it may comprise one or more alkene functionalities and/or one or more alkyne functionalities. When the hydrocarbon comprises a straight or branched chain group, it may comprise one or more primary, secondary and/or tertiary alkyl groups.

When the hydrocarbon comprises a cyclic group, it may comprise an aromatic ring, a non-aromatic ring, an aliphatic ring, a heterocyclic group, and/or fused ring derivatives of these groups. The ring may be fully saturated, partially saturated, or fully unsaturated. The cyclic group may thus comprise a benzene, naphthalene, anthracene, phenanthrene, phenalene, biphenylene, pentalene, indene, as-indacene, s-indacene, acenaphthylene, fluorene, fluoranthene, acephenanthrylene, azulene, heptalene, pyrrole, pyrazole, imidazole, 1,2,3-triazole, 1,2,4-triazole, tetrazole, pyrrolidine, furan, tetrahydrofuran, 2-aza-tetrahydrofuran, 3-aza-tetrahydrofuran, oxazole, isoxazole, furazan, 1,2,4-oxadiazol, 1,3,4-oxadiazole, thiophene, isothiazole, thiazole, thiolane, pyridine, pyridazine, pyrimidine, pyrazine, piperidine, 2-azapiperidine, 3-azapiperidine, piperazine, pyran, oxetan-2-yl, oxetan-3-yl, tetrahydropyran, 2-azapyran, 3-azapyran, 4-azapyran, 2-aza-tetrahydropyran, 3-aza-tetrahydropyran, morpholine, thiopyran, 2-azathiopyran, 3-azathiopyran, 4-azathiopyran, thiane, indole, indazole, benzimidazole, 4-azaindole, 5-azaindole, 6-azaindole, 7-azaindole, isoindole, 4-azaisoindole, 5-azaisoindole, 6-azaisoindole, 7-azaisoindole, indolizine, 1-azaindolizine, 2-azaindolizine, 3-azaindolizine, 5-azaindolizine, 6-azaindolizine, 7-azaindolizine, 8-azaindolizine, 9-azaindolizine, purine, carbazole, carboline, benzofuran, isobenzofuran, benzothiophene, isobenzothiophene, quinoline, cinnoline, quinazoline, quinoxaline, 5-azaquinoline, 6-azaquinoline, 7-azaquinoline, isoquinoline, phthalazine, 6-azaisoquinoline, 7-azaisoquinoline, pteridine, chromene, isochromene, acridine, phenanthridine, perimidine, phenanthroline, phenoxazine, xanthene, phenoxanthiin, and/or thianthrene, as well as regioisomers of the above groups. These groups may generally be attached at any point in the group, and also may be attached at a hetero-atom or at a carbon atom. In some instances particular attachment points are preferred, such as at 1-yl, 2-yl and the like, and these are specified explicitly where appropriate. All tautomeric ring forms are included in these definitions. For example pyrrole is intended to include 1H-pyrrole, 2H-pyrrole and 3H-pyrrole.

The number of carbon atoms in the hydrocarbon group is not especially limited, but preferably the hydrocarbon group comprises from 1-40 C atoms. The hydrocarbon group may thus be a lower hydrocarbon (1-6 C atoms) or a higher hydrocarbon (7 C atoms or more, e.g. 7-40 C atoms). The lower hydrocarbon group may be a methyl, ethyl, propyl, butyl, pentyl or hexyl group or regioisomers of these, such as isopropyl, isobutyl, tert-butyl, etc. The number of atoms in the ring of the cyclic group is not especially limited, but preferably the ring of the cyclic group comprises from 3-10 atoms, such as 3, 4, 5, 6, 7, 8, 9 or 10 atoms.

The groups comprising heteroatoms described above, as well as any of the other groups defined above, may comprise one or more heteroatoms from any of groups IIIA, IVA, VA, VIA or VIIA of the Periodic Table, such as a B, Si, N, P, O, or S atom or a halogen atom (e.g. F, Cl, Br or I). Thus the substituent may comprise one or more of any of the common functional groups in organic chemistry, such as hydroxy groups, carboxylic acid groups, ester groups, ether groups, aldehyde groups, ketone groups, amine groups, amide groups, imine groups, thiol groups, thioether groups, sulphate groups, sulphonic acid groups, sulphonyl groups, and phosphate groups etc. The substituent may also comprise derivatives of these groups, such as carboxylic acid anhydrides and carboxylic acid halides.

In addition, any substituent may comprise a combination of two or more of the substituents and/or functional groups defined above.

The invention will now be described in more detail with reference to some of the preferred embodiments.

The rings A and B of the compounds of the present invention form a bicyclic fused ring structure (which may comprise further fused rings when the substituents on either ring themselves form a ring). Each of rings A and B are not necessarily limited, provided that they do not prevent the PARP7 inhibitory function from occurring. Ring A and ring B may independently be comprised of an aromatic ring, a non-aromatic ring, an aliphatic ring, and/or a heterocyclic ring. The rings may be fully saturated, partially saturated, or fully unsaturated. Each ring may thus independently comprise a benzene, naphthalene, anthracene, phenanthrene, phenalene, biphenylene, pentalene, indene, as-indacene, s-indacene, acenaphthylene, fluorene, fluoranthene, acephenanthrylene, azulene, heptalene, pyrrole, pyrazole, imidazole, 1,2,3-triazole, 1,2,4-triazole, tetrazole, pyrrolidine, furan, tetrahydrofuran, 2-aza-tetrahydrofuran, 3-aza-tetrahydrofuran, oxazole, isoxazole, furazan, 1,2,4-oxadiazol, 1,3,4-oxadiazole, thiophene, isothiazole, thiazole, thiolane, pyridine, pyridazine, pyrimidine, pyrazine, piperidine, 2-azapiperidine, 3-azapiperidine, piperazine, pyran, tetrahydropyran, 2-azapyran, 3-azapyran, 4-azapyran, 2-aza-tetrahydropyran, 3-aza-tetrahydropyran, morpholine, thiopyran, 2-azathiopyran, 3-azathiopyran, 4-azathiopyran, thiane, indole, indazole, benzimidazole, 4-azaindole, 5-azaindole, 6-azaindole, 7-azaindole, isoindole, 4-azaisoindole, 5-azaisoindole, 6-azaisoindole, 7-azaisoindole, indolizine, 1-azaindolizine, 2-azaindolizine, 3-azaindolizine, 5-azaindolizine, 6-azaindolizine, 7-azaindolizine, 8-azaindolizine, 9-azaindolizine, purine, carbazole, carboline, benzofuran, isobenzofuran, benzothiophene, isobenzothiophene, quinoline, cinnoline, quinazoline, quinoxaline, 5-azaquinoline, 6-azaquinoline, 7-azaquinoline, isoquinoline, phthalazine, 6-azaisoquinoline, 7-azaisoquinoline, pteridine, chromene, isochromene, acridine, phenanthridine, perimidine, phenanthroline, phenoxazine, xanthene, phenoxanthiin, and/or thianthrene, as well as regioisomers of the above groups. These rings may generally be substituted at any point in the group, and also may be substituted at a hetero-atom or at a carbon atom. All tautomeric ring forms are included in these definitions. For example pyrrole is intended to include 1H-pyrrole, 2H-pyrrole and 3H-pyrrole.

In typical embodiments, the invention provides a compound as defined above, wherein ring B is selected from the following:

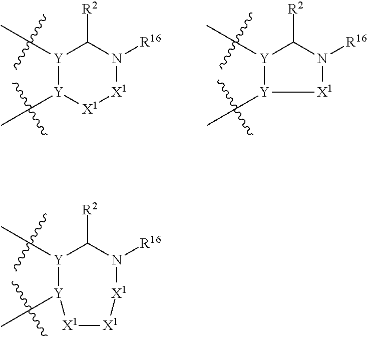

wherein each Y may independently be selected from C and N; each X¹ may independently be selected from C, N, O and S; the bonds between all of the atoms in ring B may independently be single bonds or double bonds provided that when X¹ is O or S the bonds to that X¹ are single bonds; wherein each X¹ may independently be unsubstituted, or substituted by H or a substituted or unsubstituted organic group; and wherein R¹⁶ may be present or absent and is as defined herein.

In some preferred embodiments, ring B may be selected from the following:

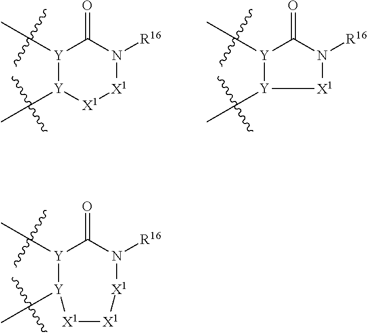

wherein Y, X¹ and R¹⁶ are as defined anywhere herein, and the bonds between all of the atoms in ring B may independently be single bonds or double bonds provided that when X¹ is O or S the bonds to that X¹ are single bonds.

In further preferred embodiments, ring B may be selected from the following:

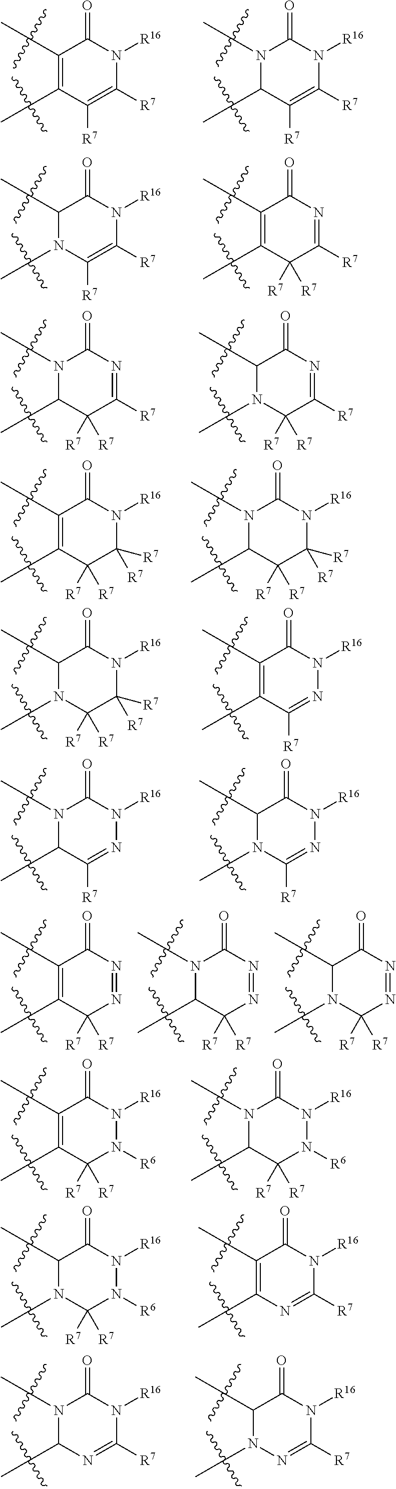

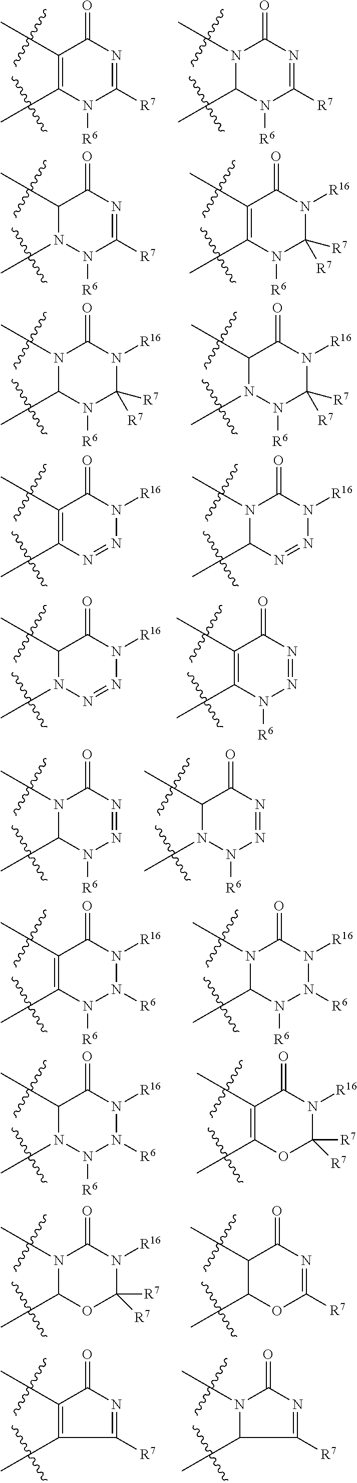

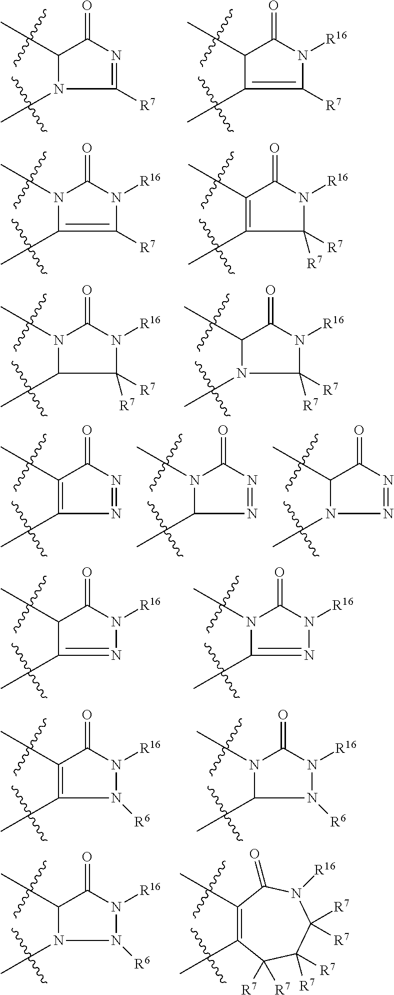

wherein R⁶ and R⁷ are independently selected from H or a substituted or unsubstituted organic group and R¹⁶ is as defined herein.

In further preferred embodiments, and independently from ring B, ring A may be selected from the following:

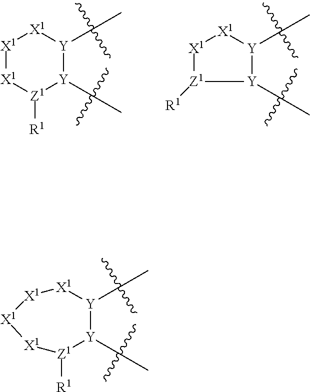

wherein Y, X¹, Z¹ and R¹ are as defined herein.

In yet further preferred embodiments, ring A may be selected from the following:

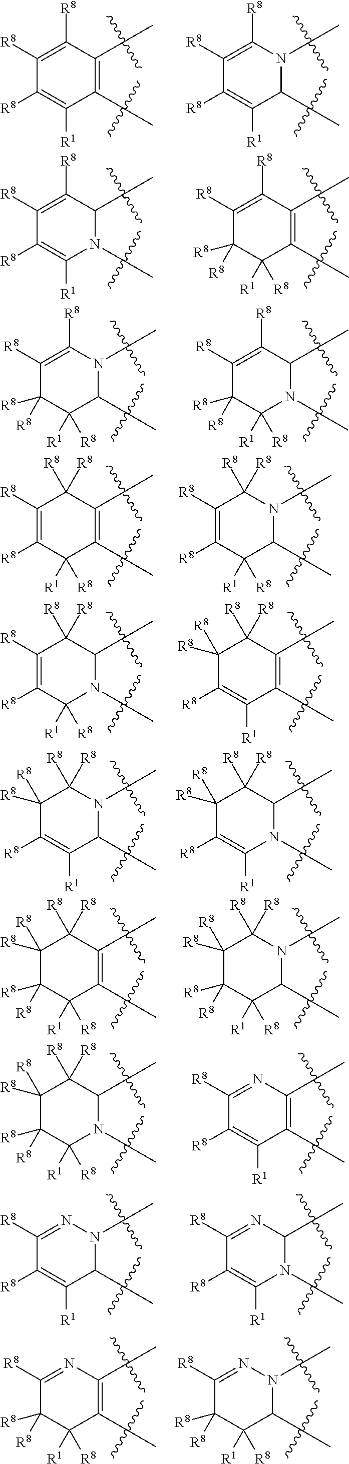

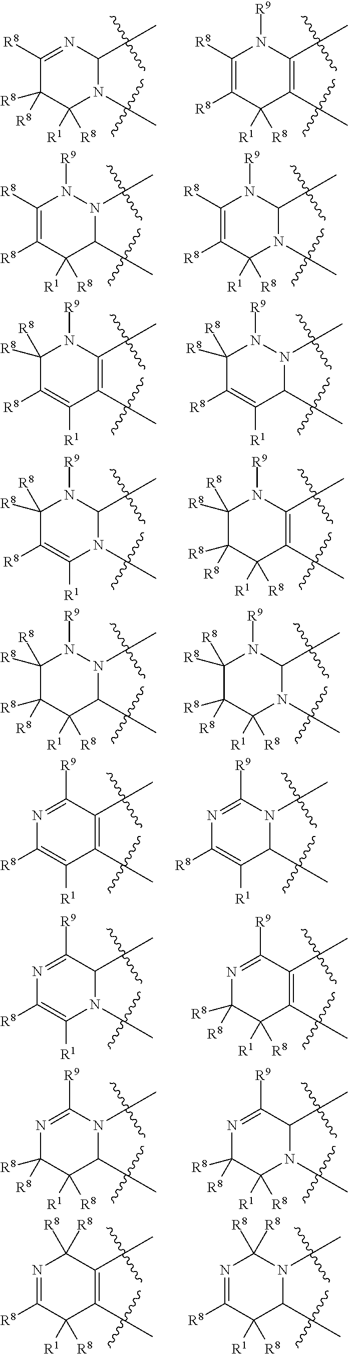

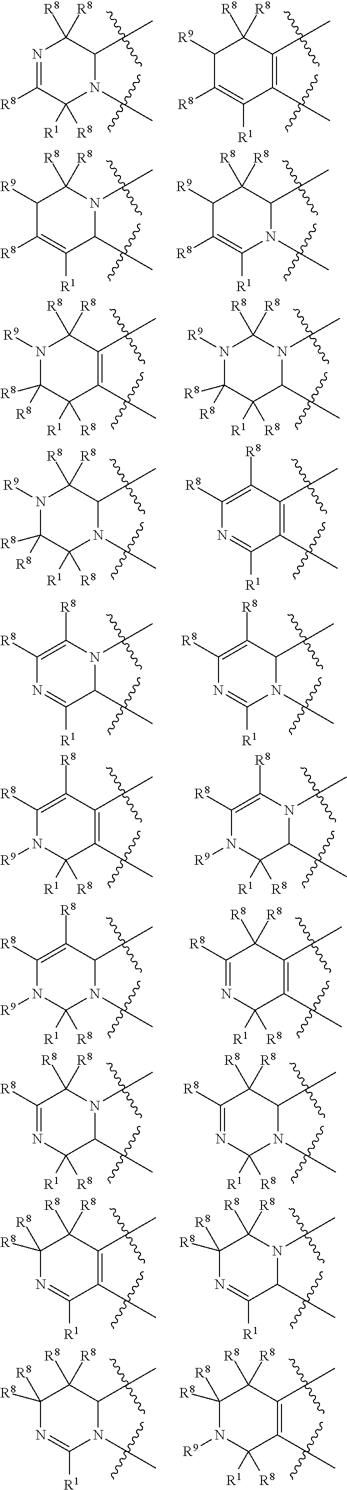

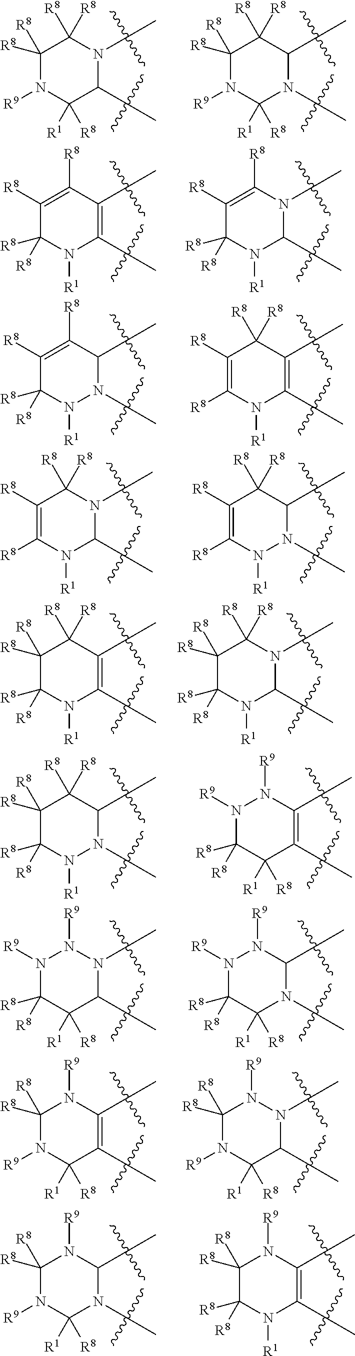

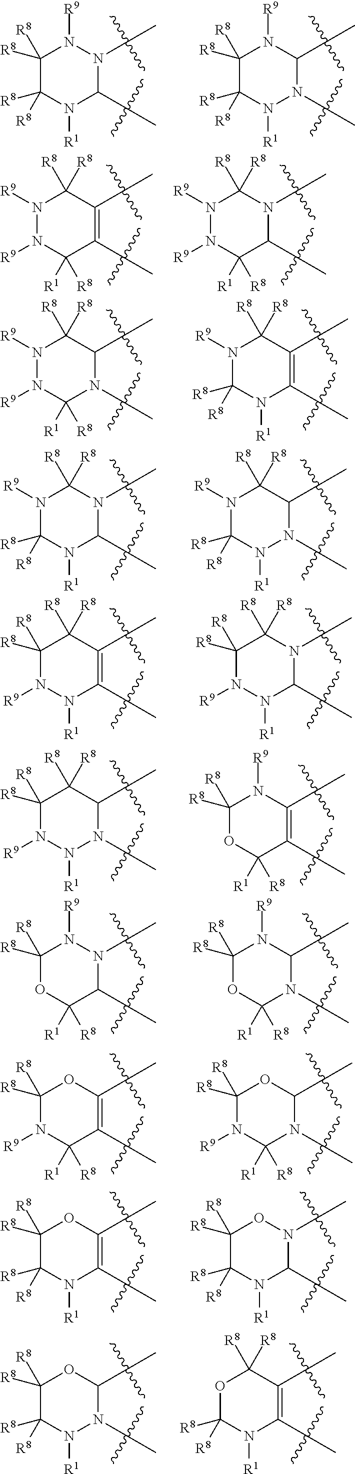

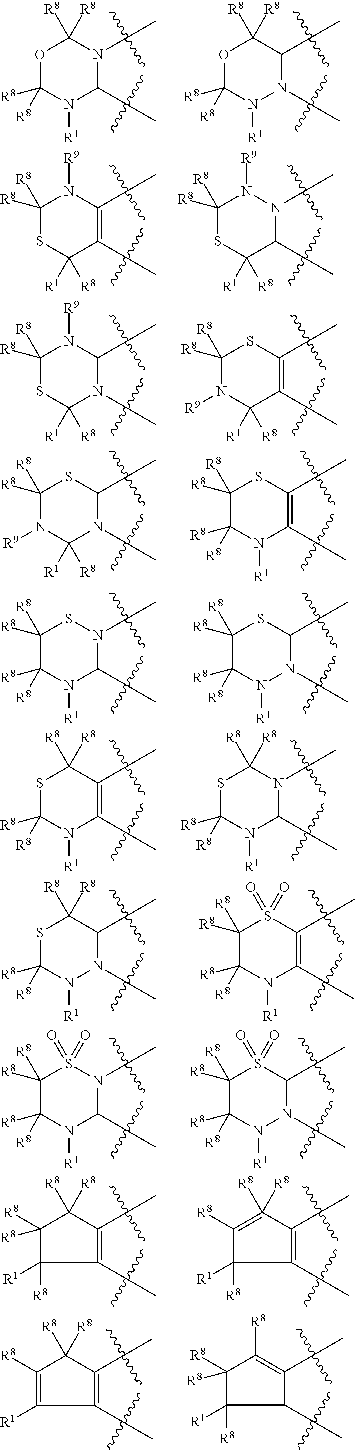

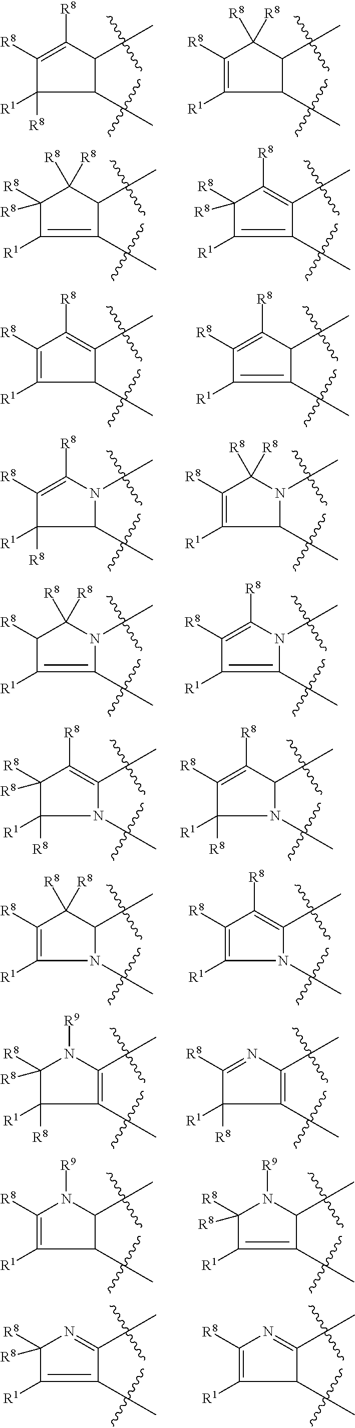

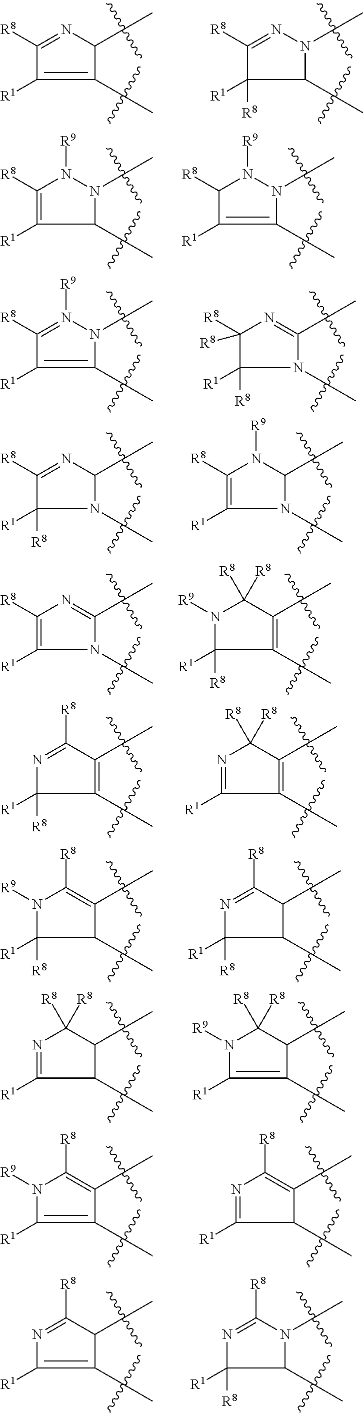

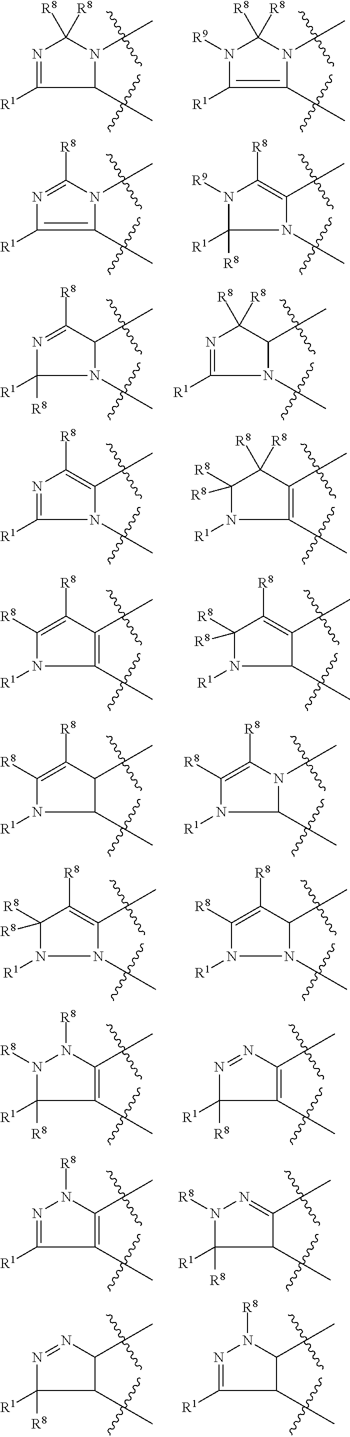

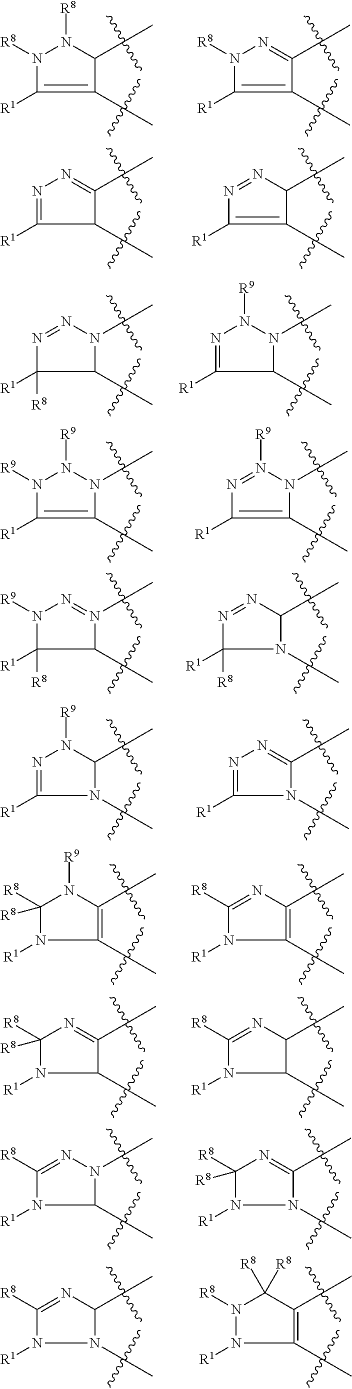

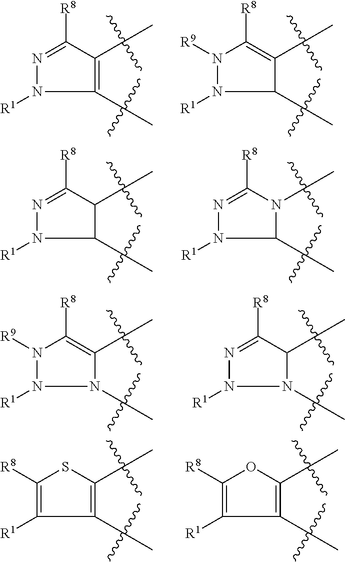

wherein R¹ is as defined herein, and R⁸ and R⁹ are independently selected from H and a substituted or unsubstituted organic group.

In preferred embodiments, and independently from ring A and ring B, R¹ may be selected from the following:

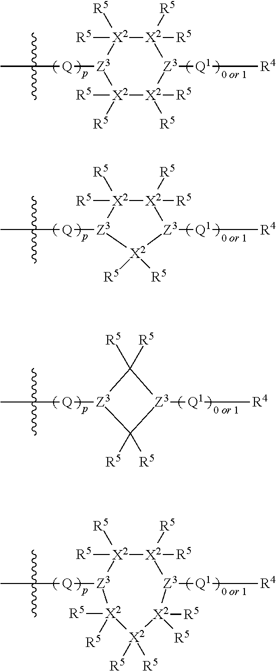

wherein Q, Q¹, p, Z³, X², R⁴ and R⁵ are as defined herein.

In still further preferred embodiments, R¹ may be selected from the following:

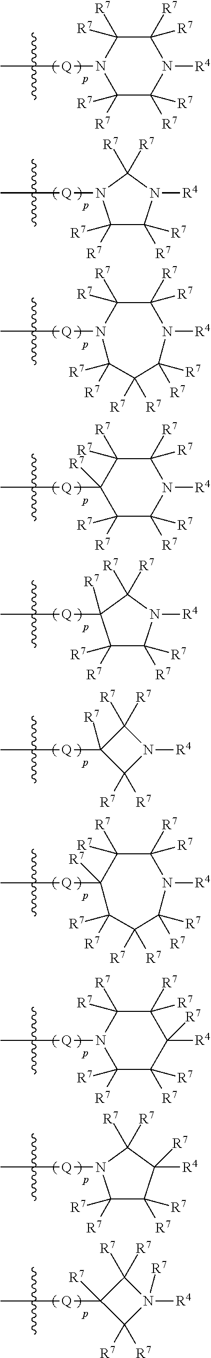

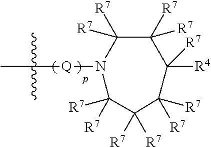

wherein Q, p, R⁷ and R⁴ are as defined herein.

In some preferred embodiments, the linking group -(Q)ₚ- may be selected from the following:

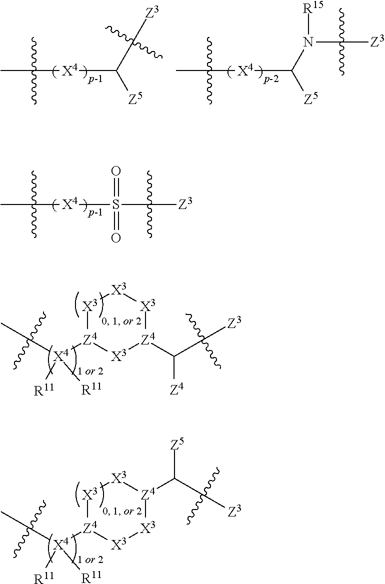

wherein each X³ may be the same or different and is independently selected from C, N, O and S; when C or N, each X³ may independently be unsubstituted or substituted with H or a substituted or unsubstituted organic group; each X⁴ may be the same or different and is independently selected from C, N, O and S; each Z⁴ may be the same or different and is independently selected from C and N; the bonds between all of the atoms any ring may independently be single bonds or double bonds provided that when X³ is O or S the bonds to that X³ are single bonds; R¹¹ may be present or absent depending on the number of bonds and the valence of the X⁴ atom comprising that R¹; and wherein each R¹ is independently selected from H or a substituted or unsubstituted organic group; and wherein R¹⁵ is selected from H, a linear or branched C₁-C₆ alkyl group or a linear or branched C₁-C₆ halogenated alkyl group; and wherein Z⁵ may be attached via a single bond or a double bond and is selected from the following:

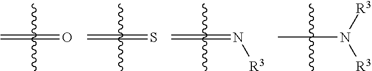

wherein each R³ may be the same or different and is independently selected from H and a substituted or unsubstituted organic group;
and wherein p and Z³ are as defined herein.

In still further preferred embodiments, the linking group -(Q)ₚ- may be selected from the following:

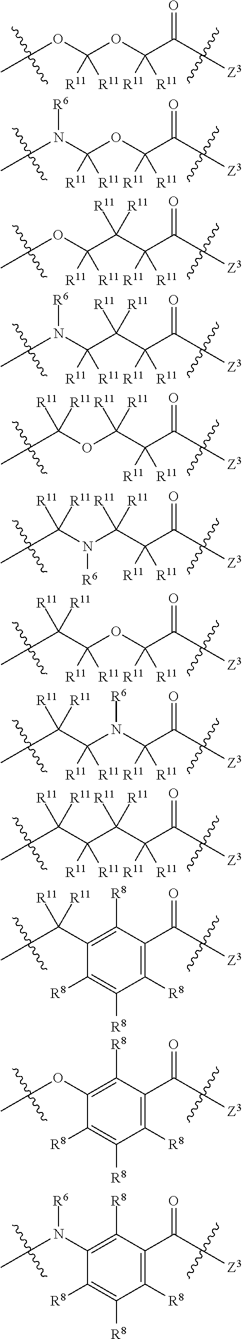

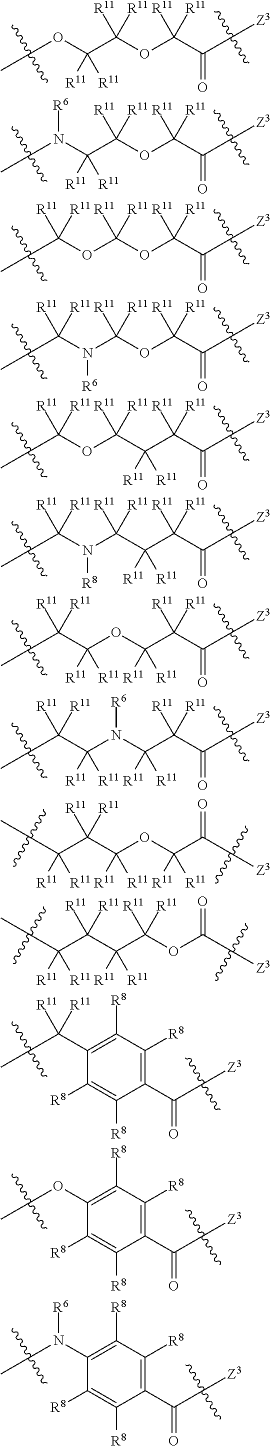

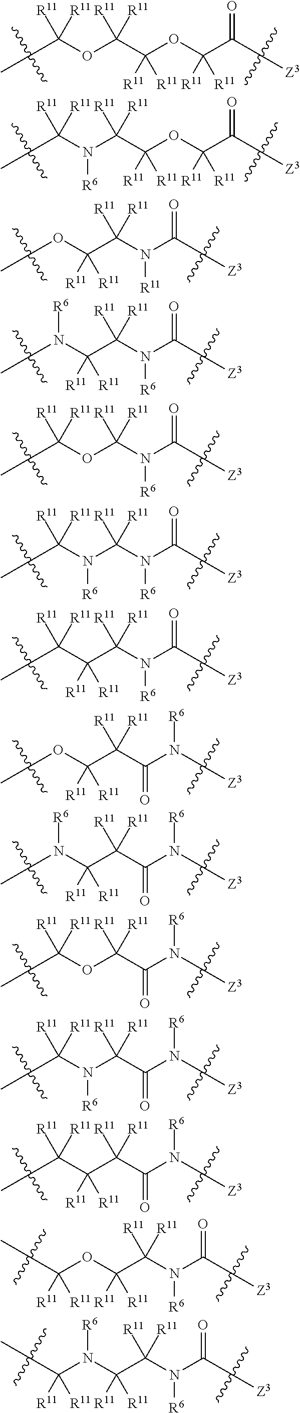

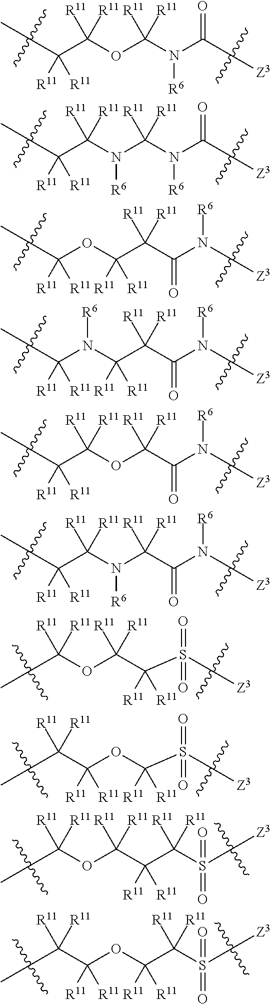

wherein Z³, R⁶, R⁸ and R¹¹ are as defined herein.

In preferred embodiments of the present invention, R⁴ may be attached via a single bond or a double bond, and may be selected from the following:

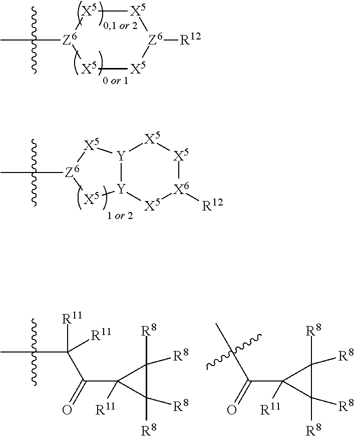

wherein each X⁵ may be the same or different and is independently selected from C, N, O and S; when C or N, each X⁵ may independently be unsubstituted or substituted with H or a substituted or unsubstituted organic group; each Z⁶ may be the same or different and is independently selected from C, N, O and S; the bonds between all of the atoms any ring may independently be single bonds or double bonds provided that when X⁵ or Z⁶ is O or S the bonds to that X⁵ or Z⁶ are single bonds; R¹² may be present or absent depending on the number of bonds and the valence of the Z⁶ atom comprising that R¹²; wherein if present R¹² is independently selected from H or a substituted or unsubstituted organic group; wherein each Z⁶ may independently be further substituted with H or a substituted or unsubstituted organic group; and wherein R⁸ and R¹¹ are as defined herein.

In some preferred embodiments, R⁴ may be selected from the following:

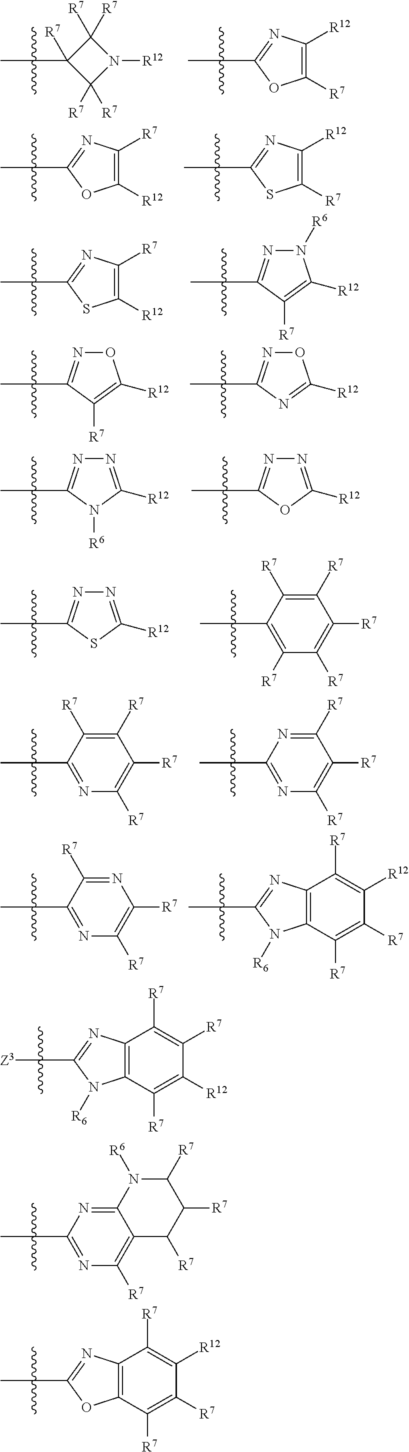

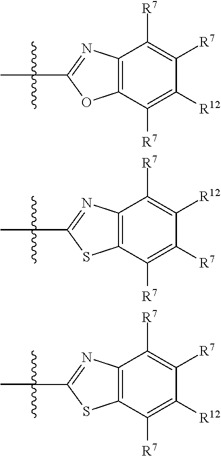

wherein R⁶, R⁷ and R¹² are each independently H or a substituted or unsubstituted organic group.
Q¹ may be present or absent and it is preferred that Q¹ is absent so that R⁴ is directly attached to Z³. When present, Q¹ is typically O, S, CH₂ or NH.

In preferred embodiments of the invention, R² may be attached via a single bond or a double bond and is selected from the following:

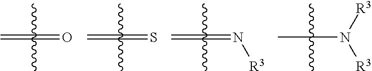

wherein each R³ may be the same or different and is independently selected from H and a substituted or unsubstituted organic group.

In some preferred embodiments R¹ may be selected from the following:

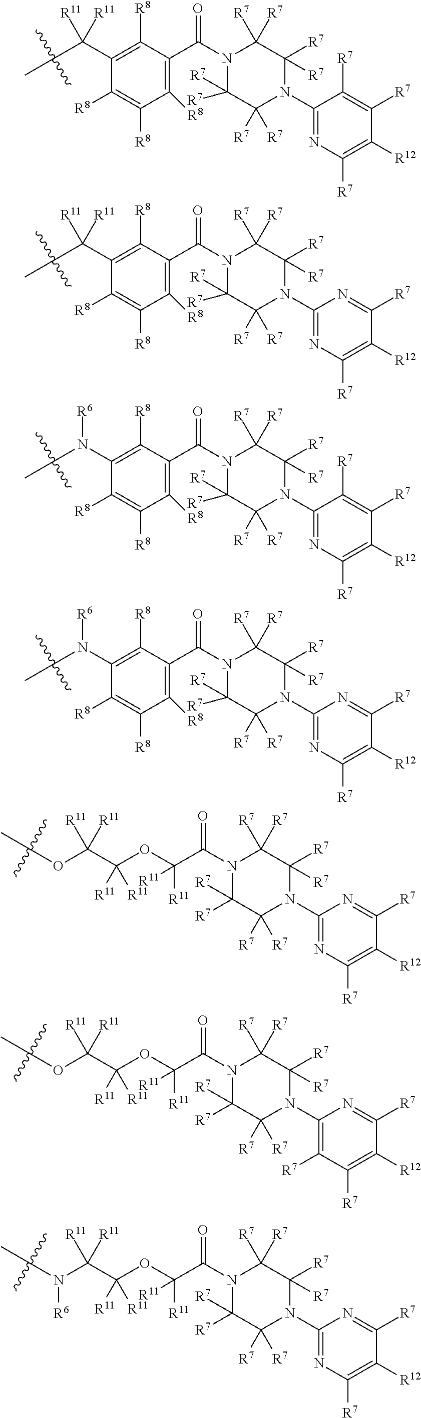

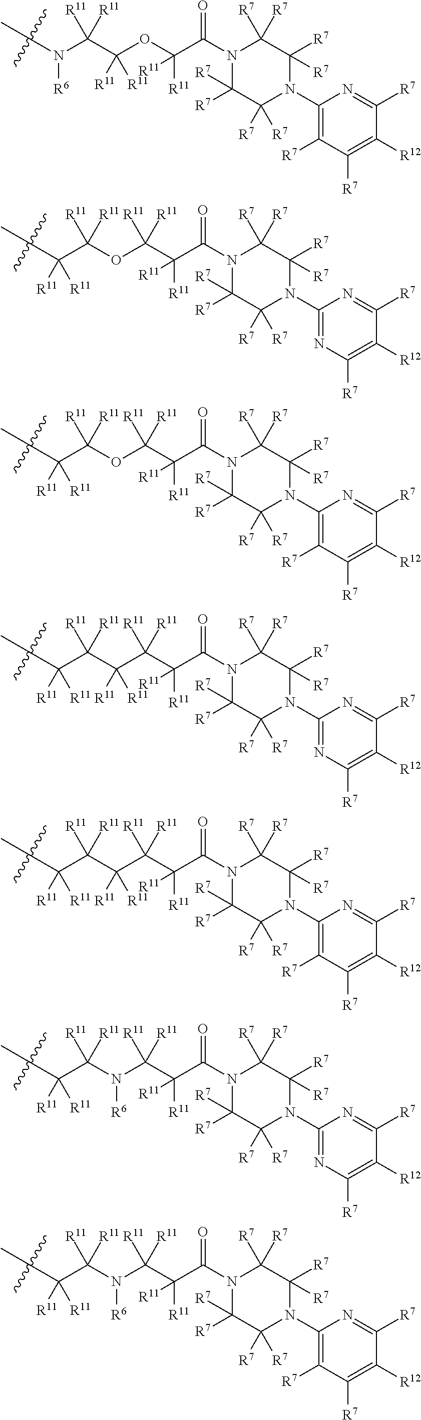

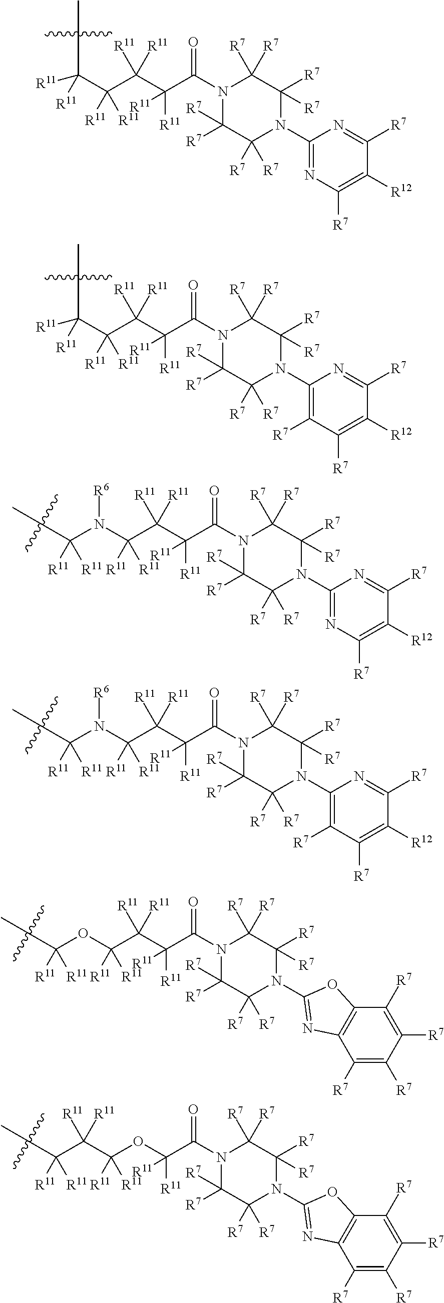

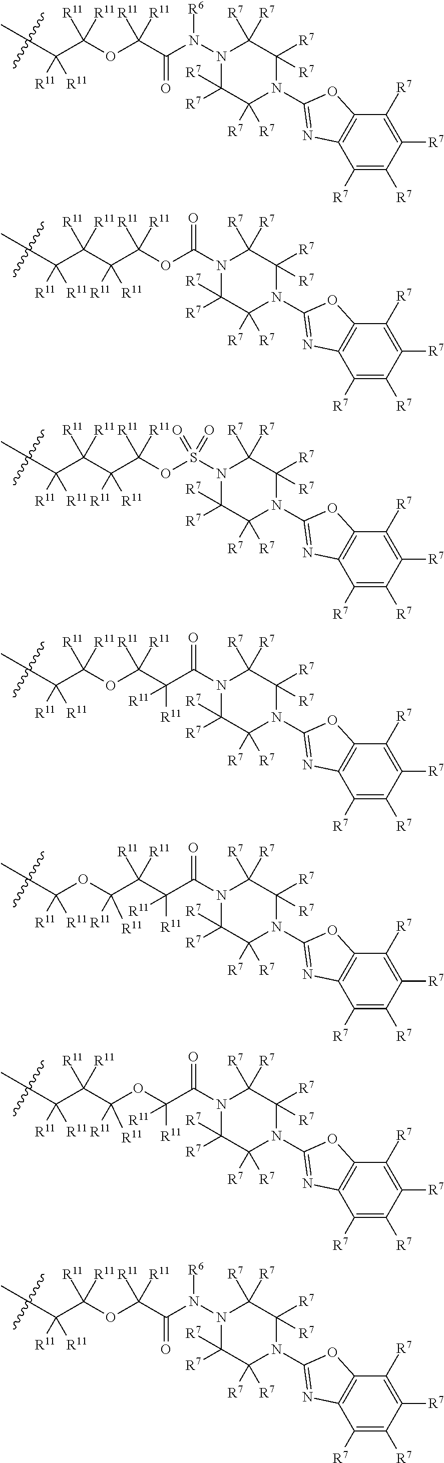

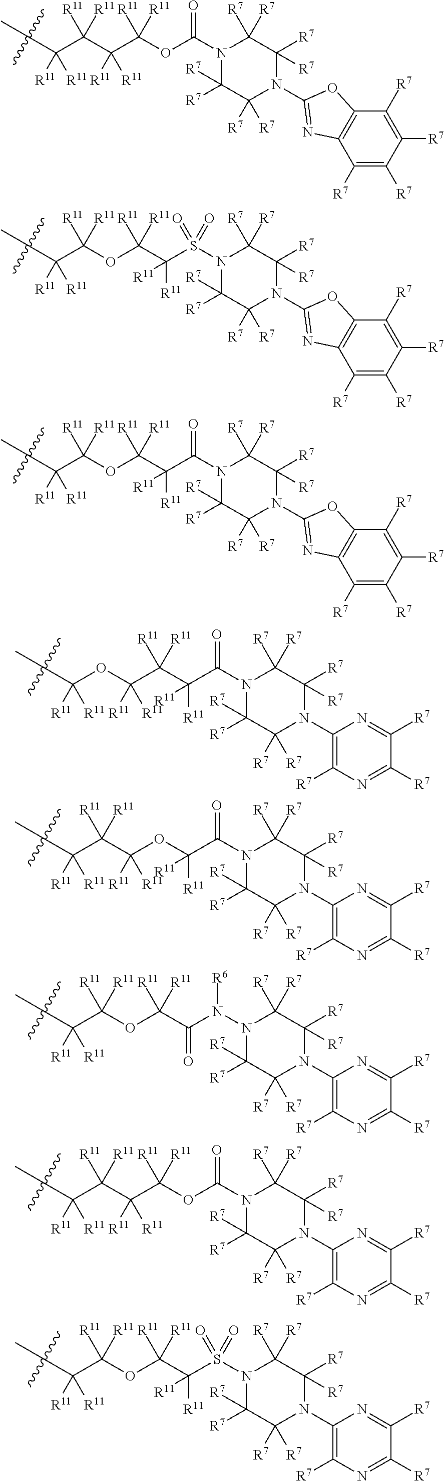

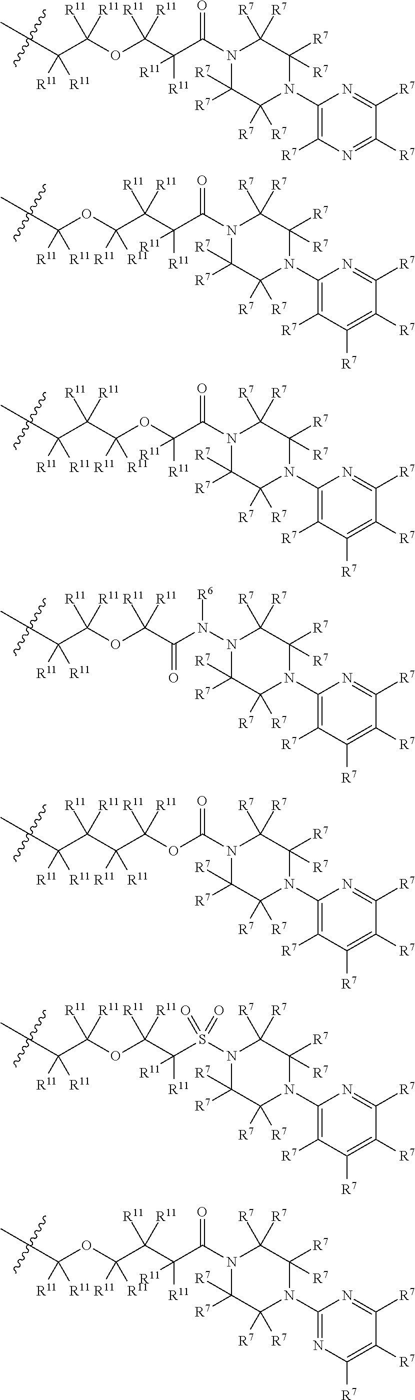

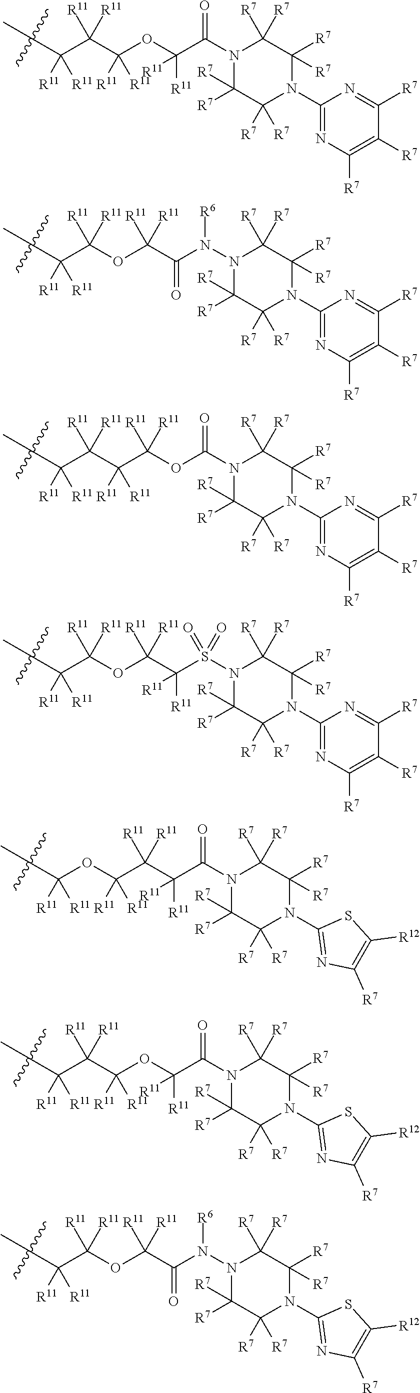

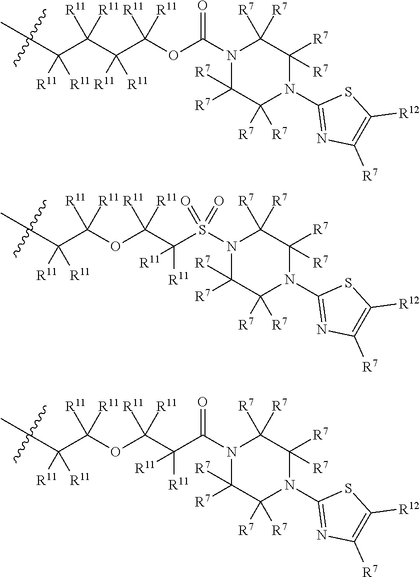

wherein R⁶, R⁷, R⁸, R¹¹ and R¹² are as defined herein.

The present invention further provides a PARP7 inhibitor compound, which compound comprises the following formula:

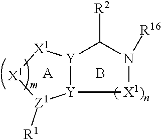

wherein each X¹ may be the same or different and is independently selected from C, N, O and S; each Y may be the same or different and is independently selected from C and N; Z¹ is independently selected from C and N; each X¹ may independently be unsubstituted, or may independently be substituted with H or a substituted or unsubstituted organic group; each Y may independently be unsubstituted, or may independently be substituted with H or a substituted or unsubstituted organic group; Z¹ may independently be further substituted with H or a substituted or unsubstituted organic group; m may be 1, 2, or 3; n may be 1, 2 or 3, preferably 1 or 2; the bonds between all of the atoms in ring A may independently be single bonds or double bonds provided that when X¹ is O or S the bonds to that X¹ are single bonds; the bonds between all of the atoms in ring B may independently be single bonds or double bonds provided that when X¹ is O or S the bonds to that X¹ are single bonds; and R² and R¹⁶ are as defined herein;
and wherein R¹ may be attached to Z¹ by a single bond or a double bond and is a substituent of formula:

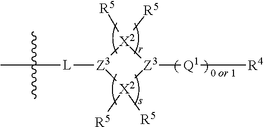

wherein L is a group selected from any of the following:

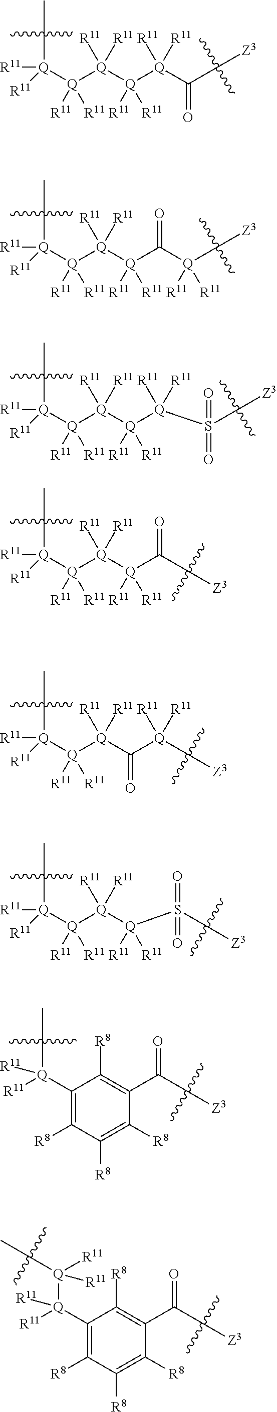

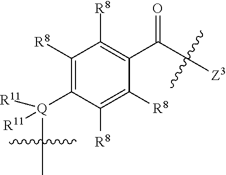

wherein each Q may be the same or different and is independently selected from C, N, O and S; each Q may independently be attached to another Q, or to Z³, by a single bond or a double bond; each Q may independently be unsubstituted, or may independently be substituted by H or a substituted or unsubstituted organic group; each R⁸ is independently selected from H and a substituted or unsubstituted organic group; R¹¹ may be present or absent depending on the number of bonds and the valence of the Q atom comprising that R¹¹; and each R¹¹ is independently selected from H and a substituted or unsubstituted organic group; and wherein each Z³ may be the same or different and is independently selected from C and N; each X² may be the same or different and is independently selected from C, N, O and S; r is a number from 1 to 3; and s is independently a number from 1 to 3; wherein Q¹ is selected from C, N, O and S and may be attached to Z³ and R⁴, by a single bond or a double bond and may be unsubstituted, or substituted by H or an organic group; each bond in the ring comprised of Z³ and X² atoms may independently be a double bond or a single bond provided that when X² is O or S the bonds to that X² are single bonds; each R⁵ may be present or absent depending on the number of bonds to, and the valence of, the X² atom attached to that R⁵; and wherein each R⁵ is independently selected from H or a substituted or unsubstituted organic group;
and wherein R⁴ is a group selected from any of the following:

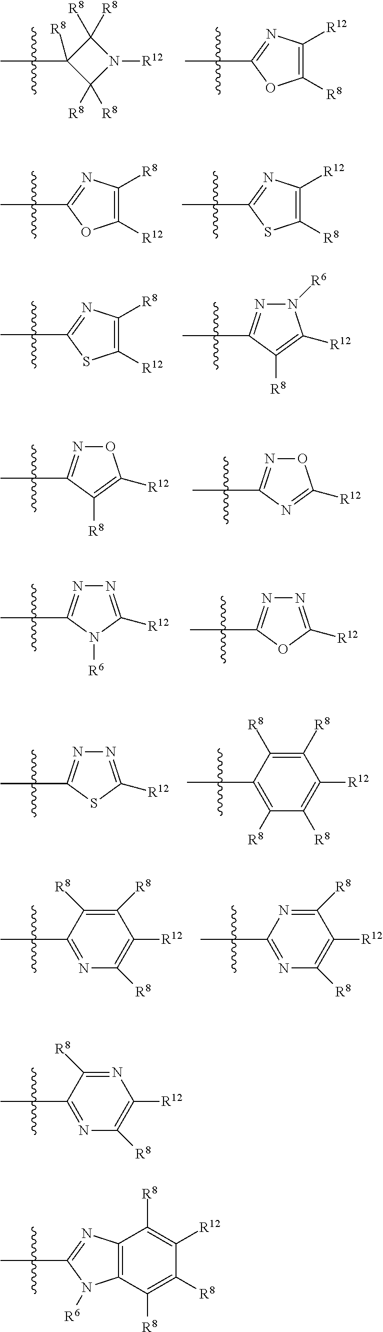

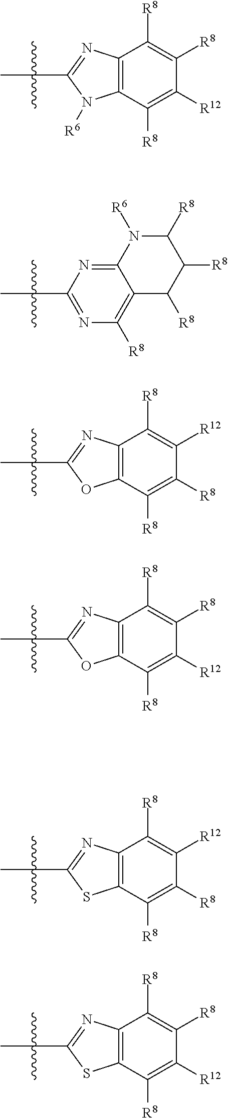

and wherein each R⁶ is independently selected from H and a substituted or unsubstituted organic group; and wherein R¹² is independently selected from H or a substituted or unsubstituted organic group and preferably a group selected from: —H, —CH₃, —CN, —CF₃, —CHF₂, —CH₂F, —OCF₃, —OMe, —CH₂CF₃, —CF₂CH₃, —OCHF₂, —OCH₂F, —F, —Cl, —Br, —I, —SO₂Me, —CONHMe, t-Bu, cyclopropyl and

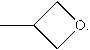

In a compound according to this formula it is preferred that R¹ has any of the following structures:

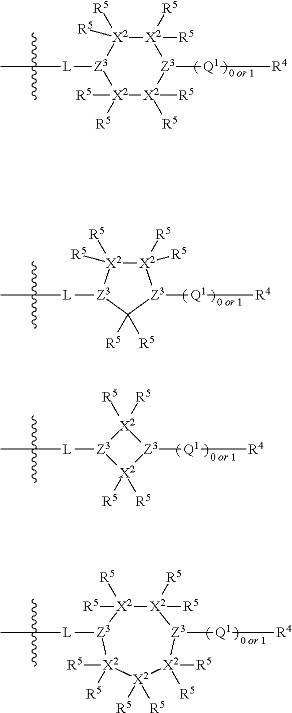

wherein L, Z³, X², Q¹, R⁴ and R are as defined herein.

Preferably, R¹ has any of the following structures:

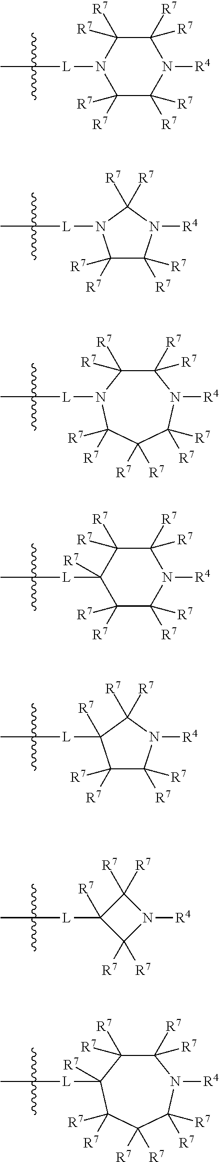

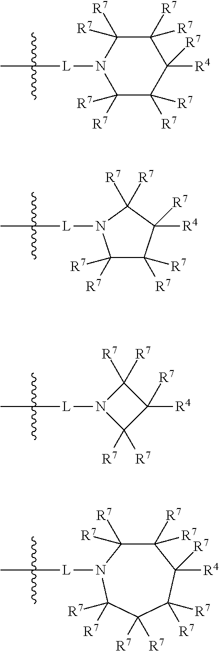

wherein L, R⁴ and R⁷ are as defined herein.

Advantageously, the compounds according to the invention may comprise the following general formula:

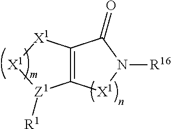

wherein X¹, Z¹, R¹, R¹⁶, m and n are as defined herein. Typically, m is 1,2 or 3; n is 1,2 or 3; m is preferably 1 or 2; and n is preferably 1 or 2, most preferably 2.

The invention will now be described in more detail. Firstly, a number of typical general structures of the compounds of the invention will be described.

The following are a number of typical general structures of the compounds of the invention.

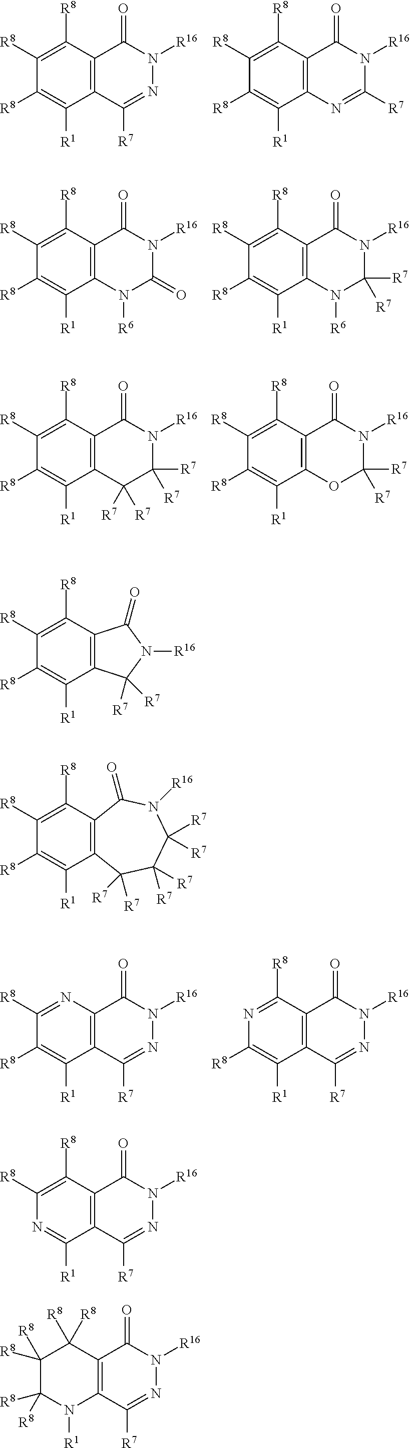

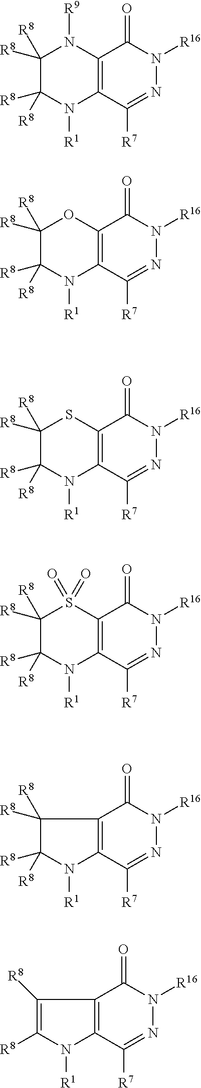

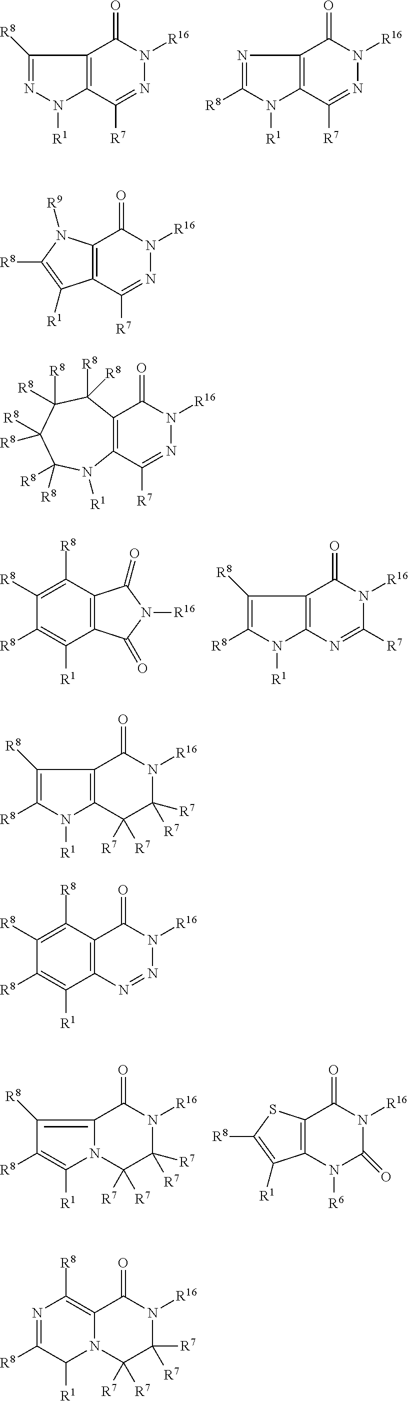

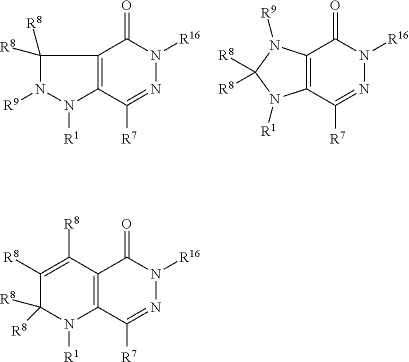

wherein R¹, R⁶, R⁷, R⁸, R⁹ and R¹⁶ are as defined herein.

The following are some preferred general structures according to the invention:

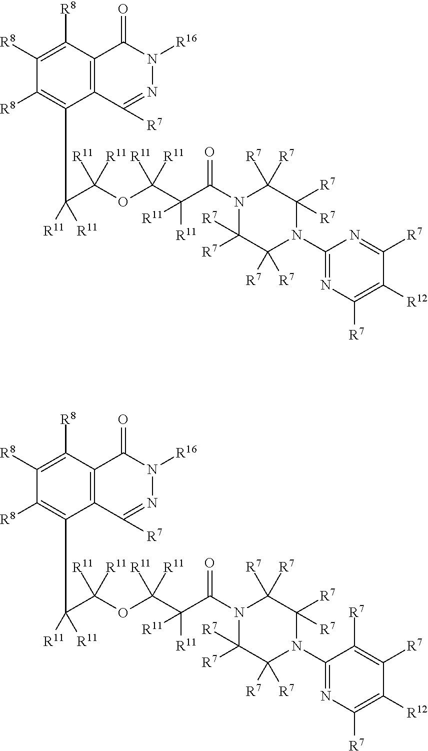

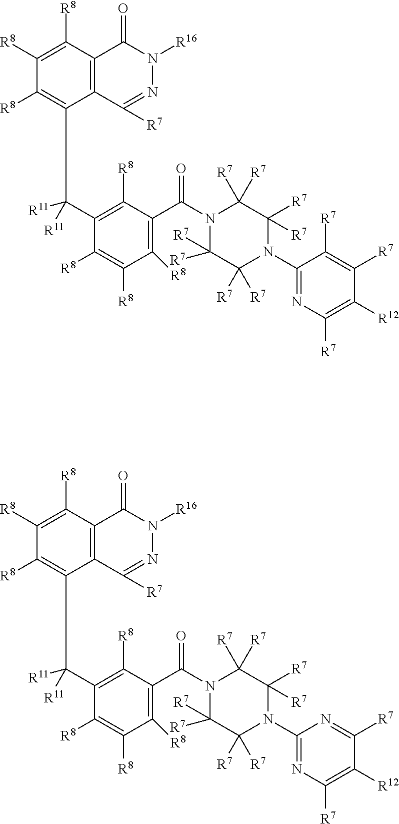

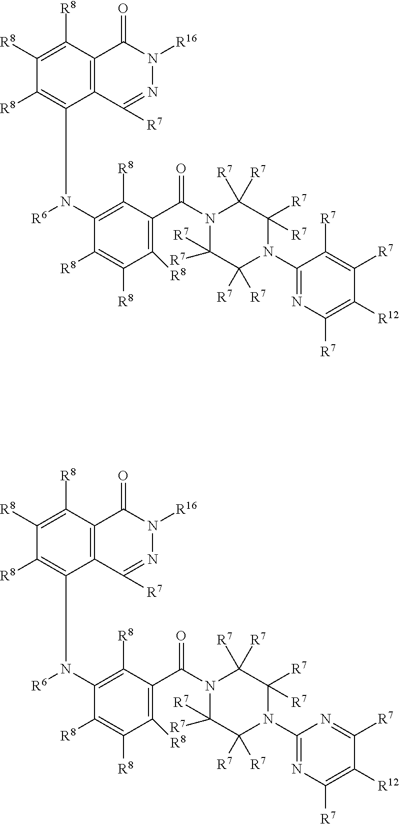

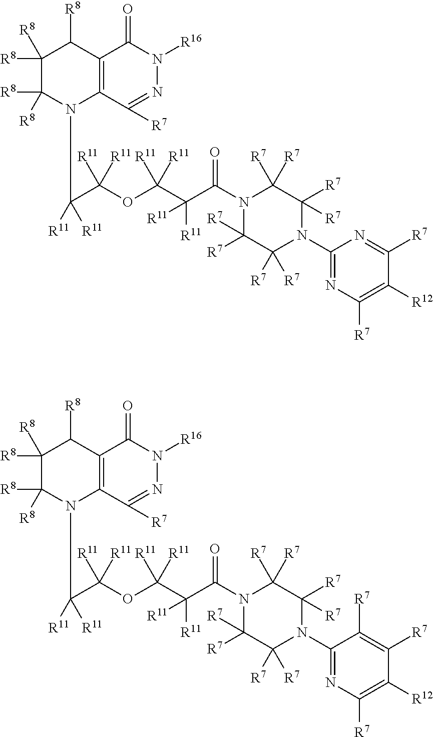

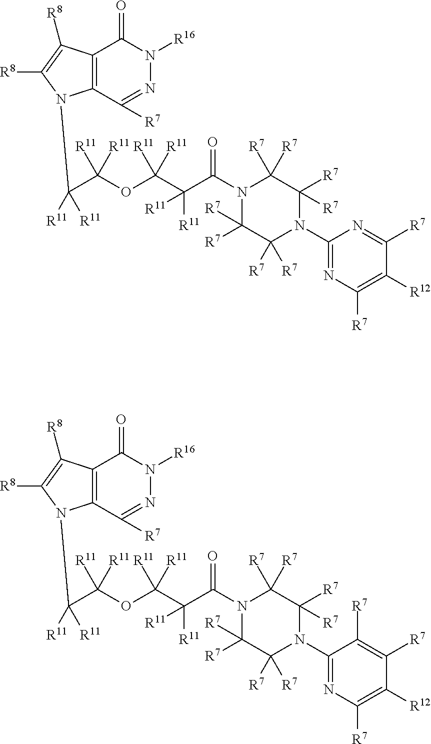

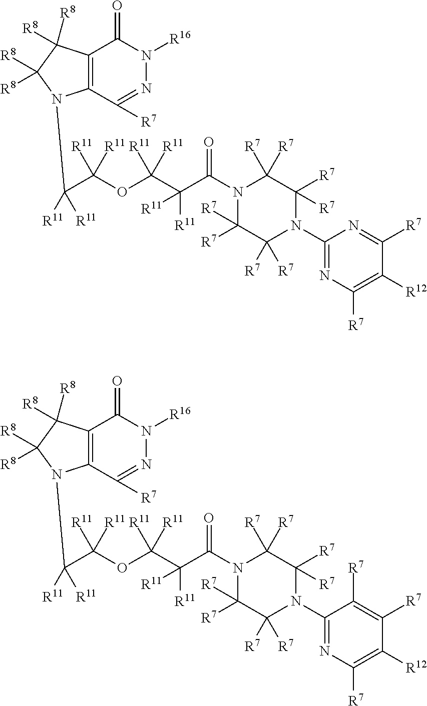

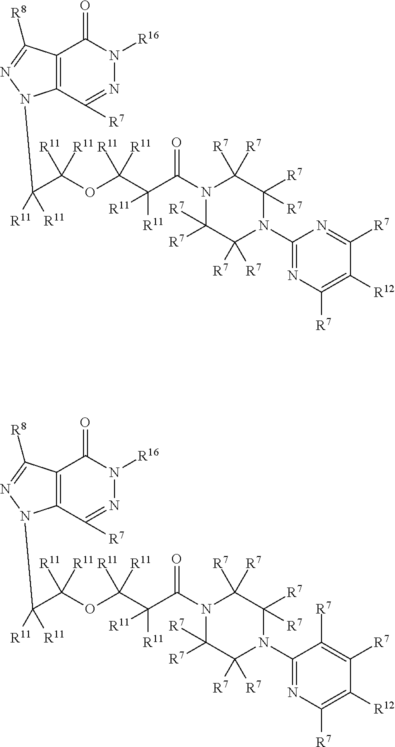

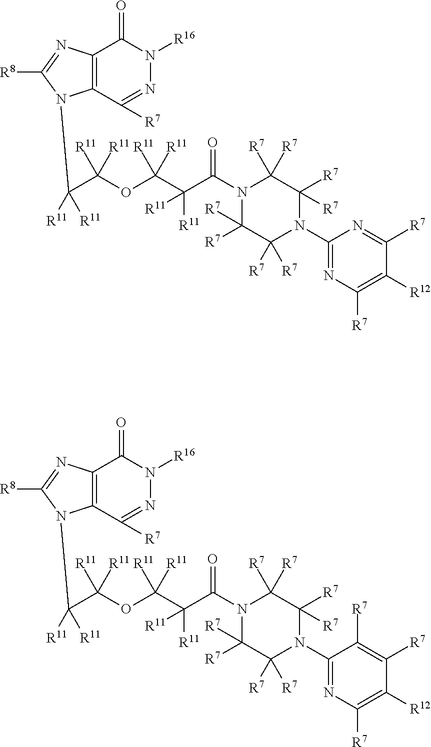

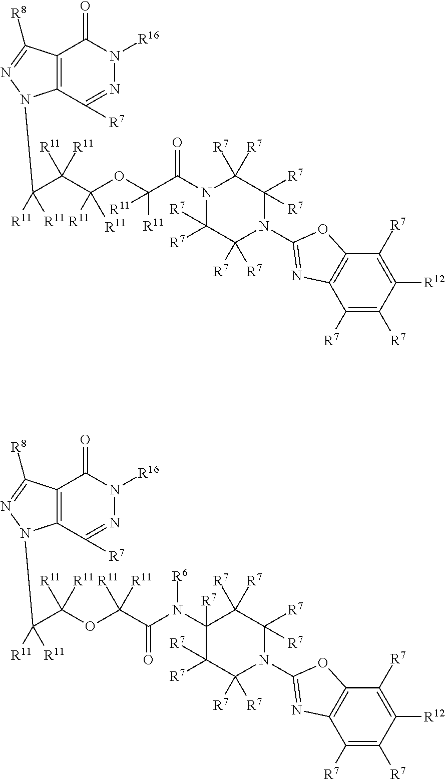

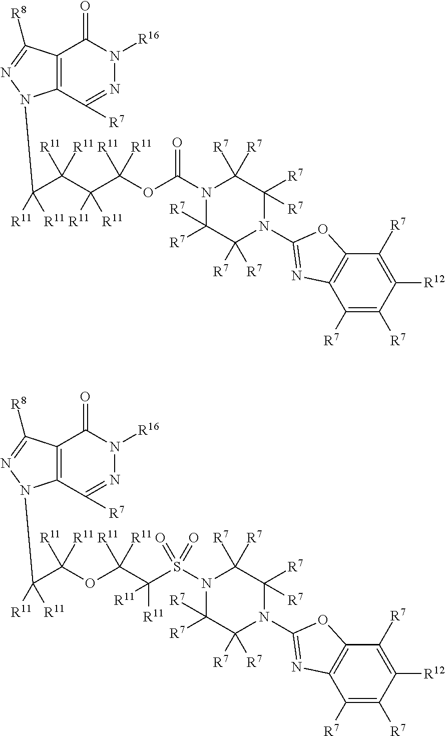

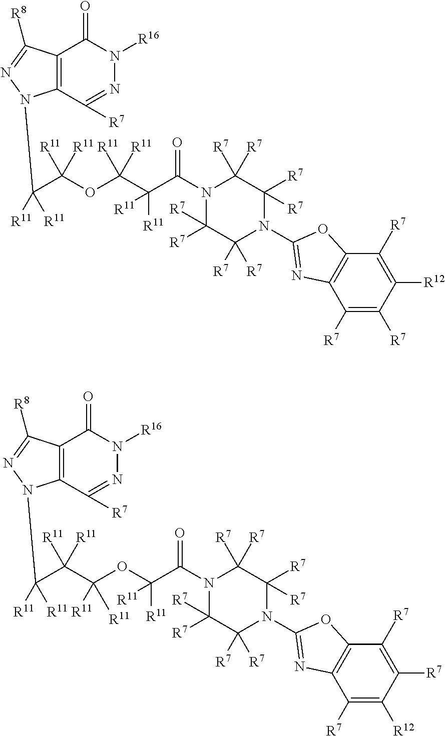

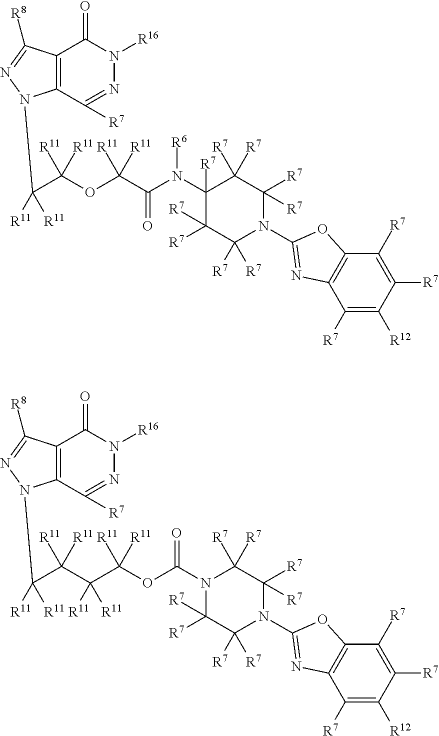

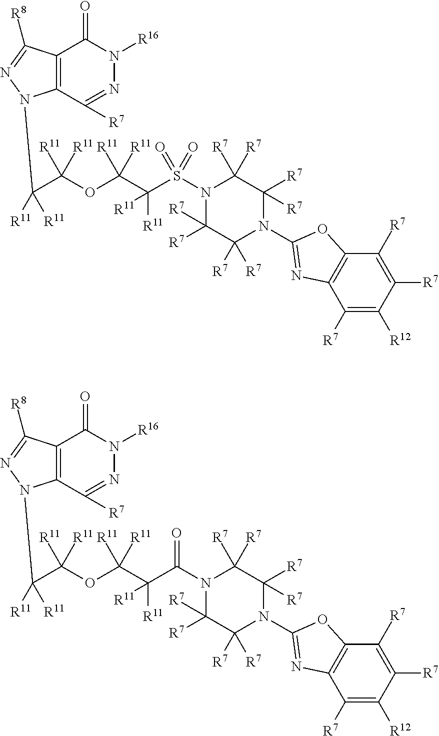

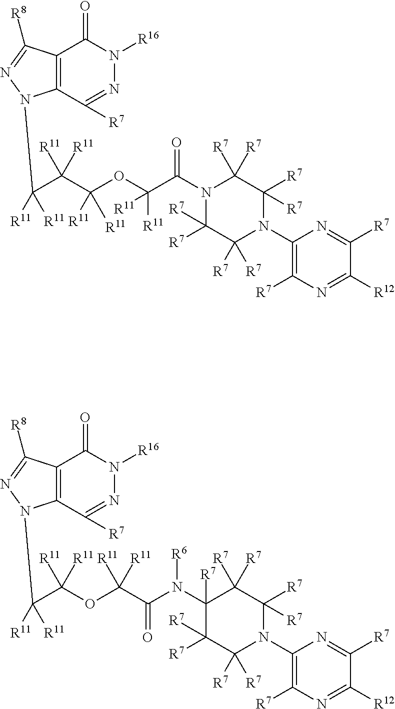

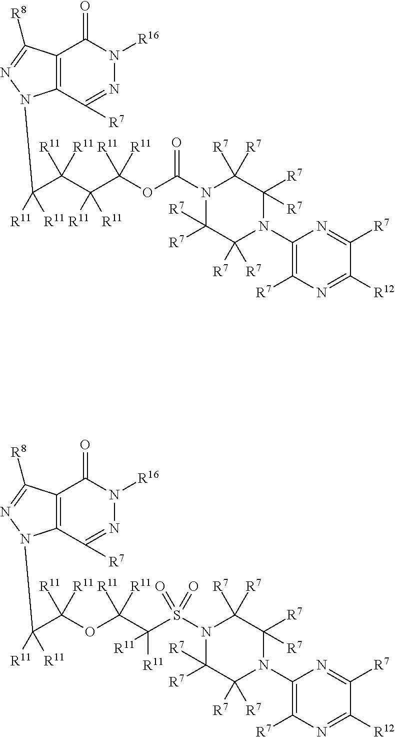

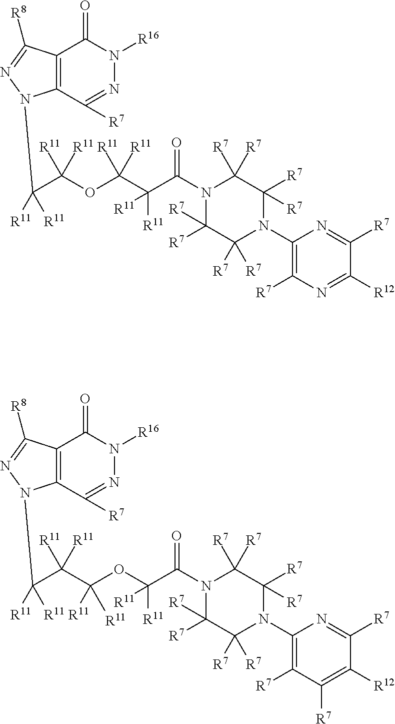

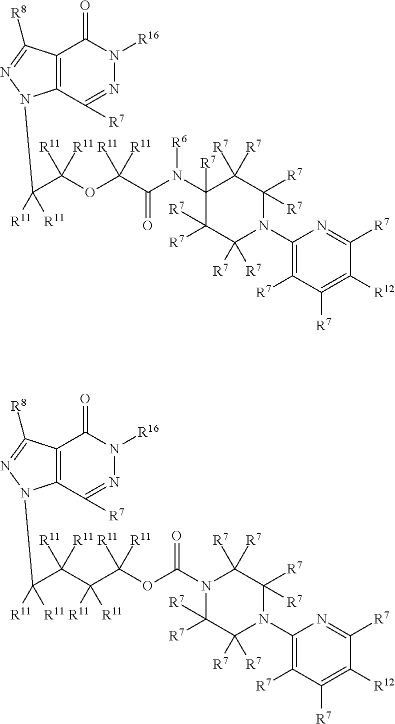

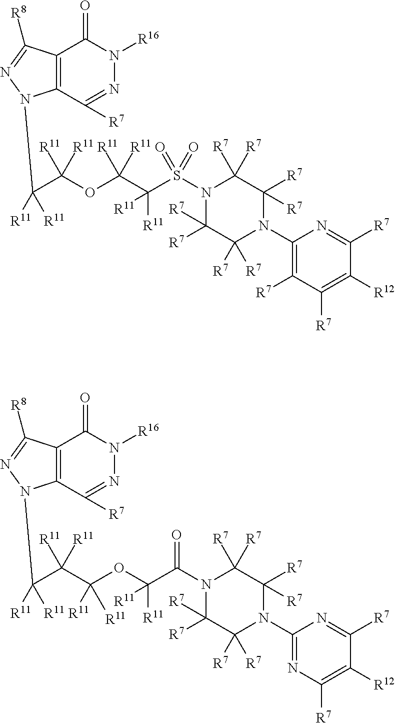

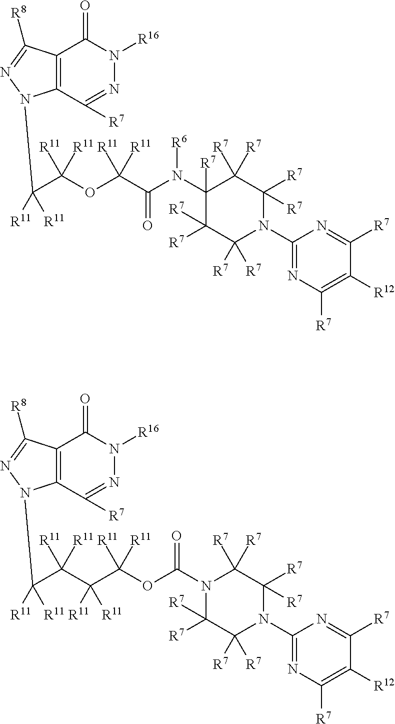

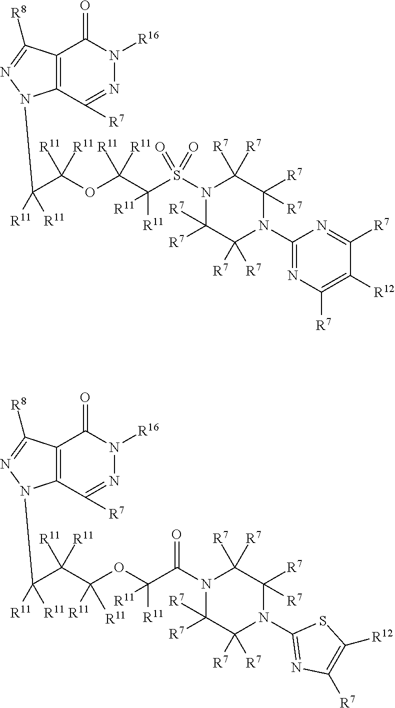

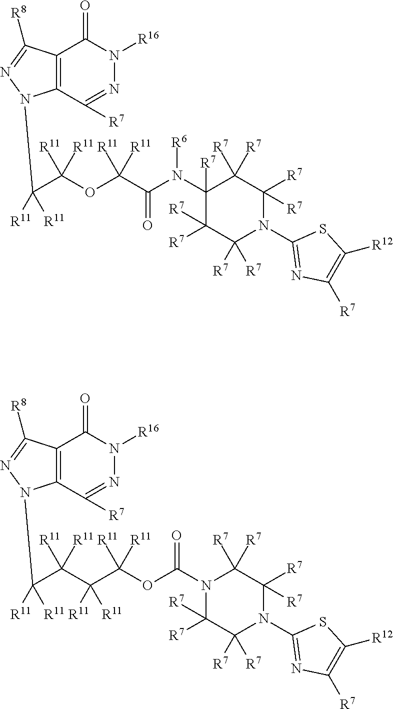

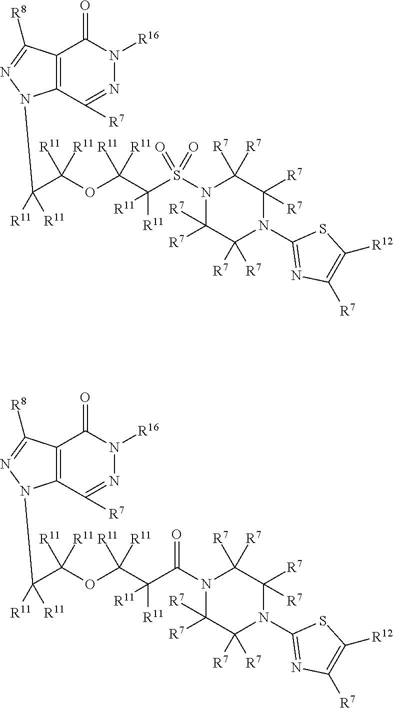

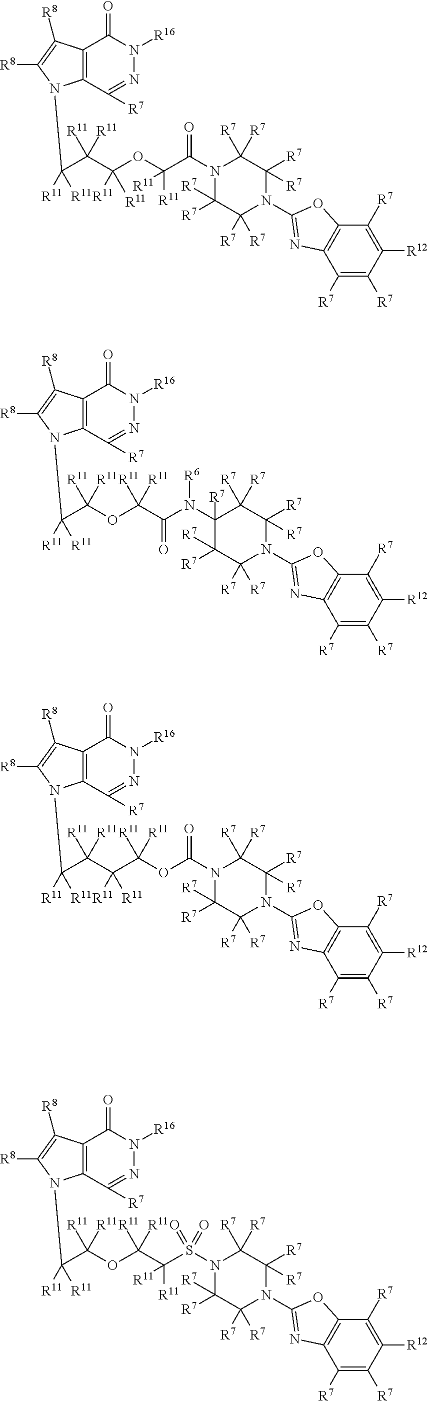

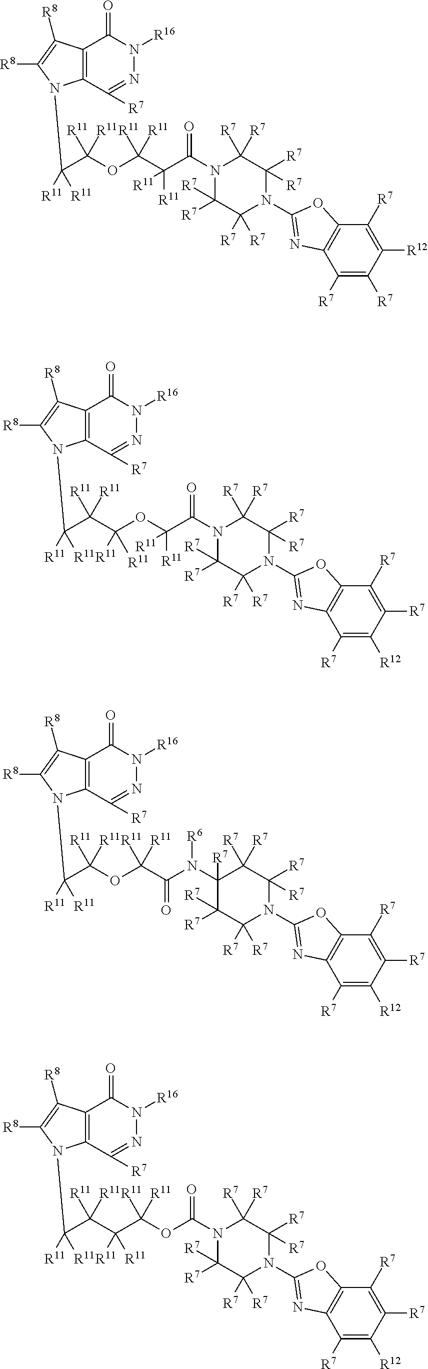

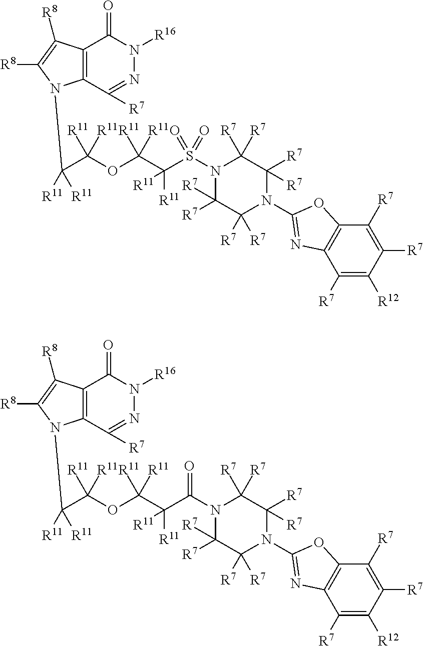

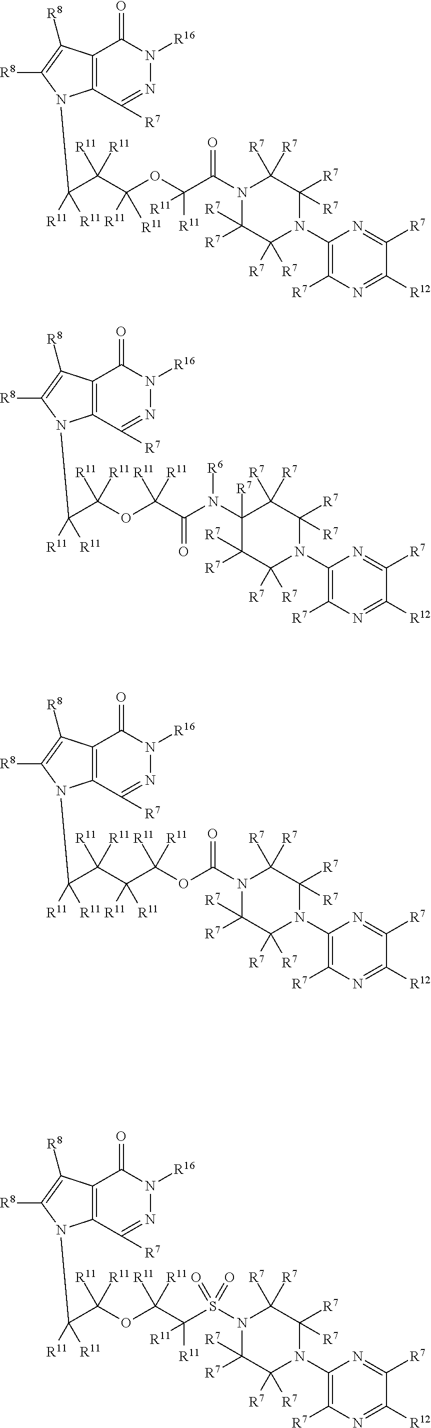

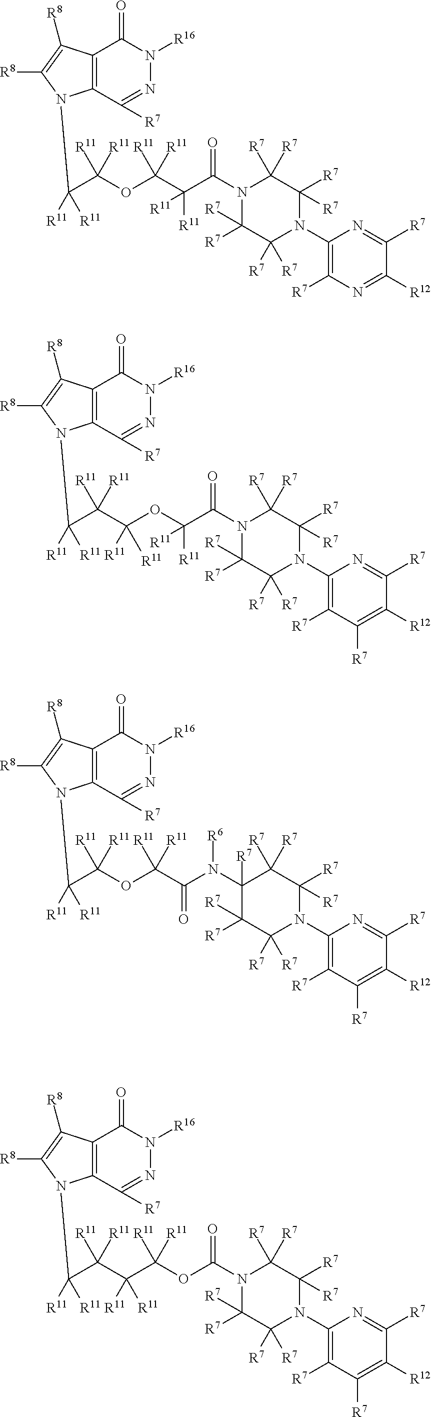

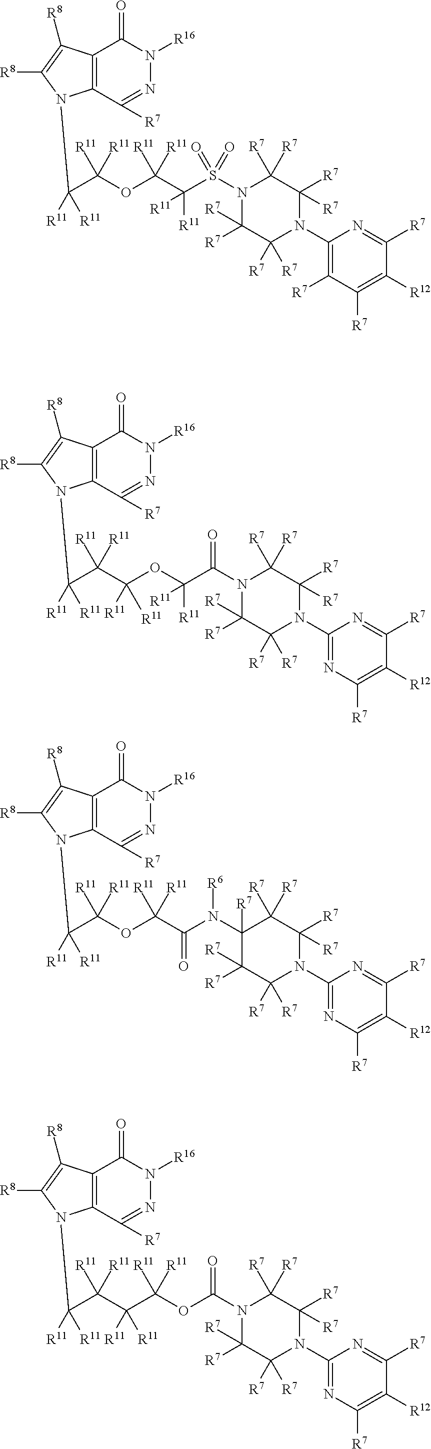

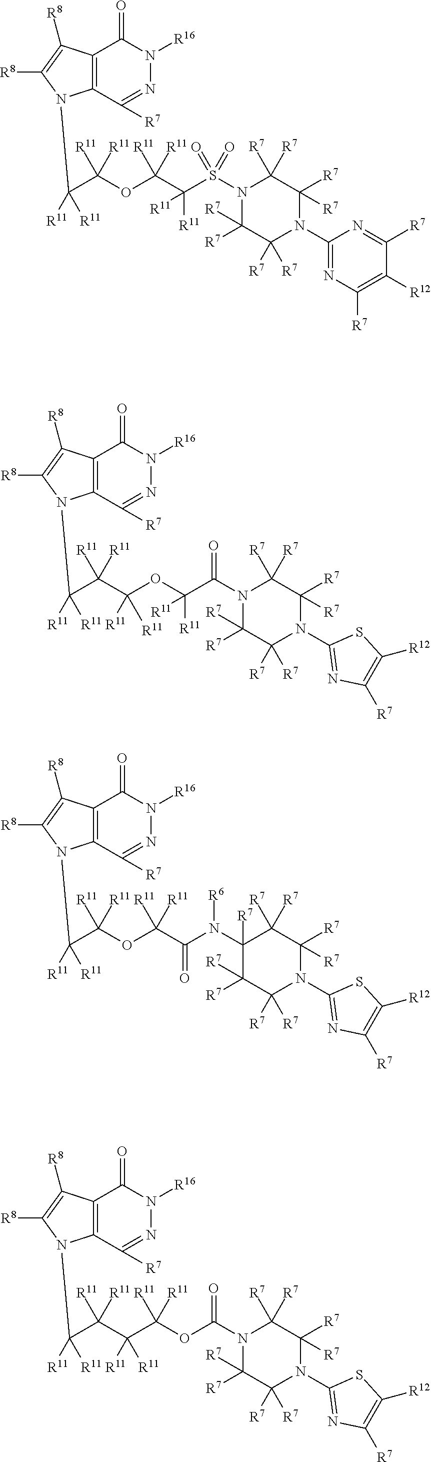

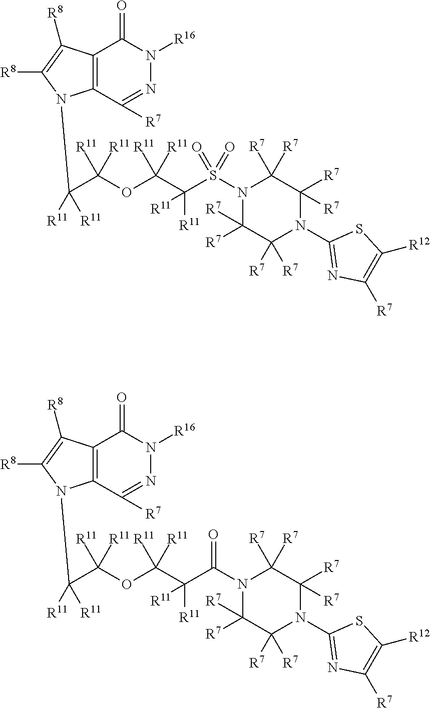

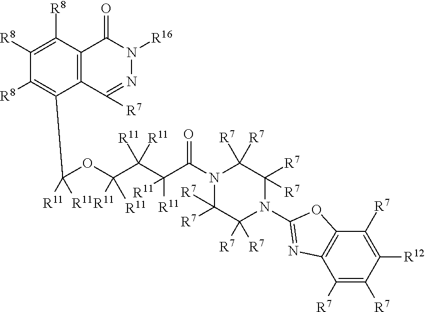

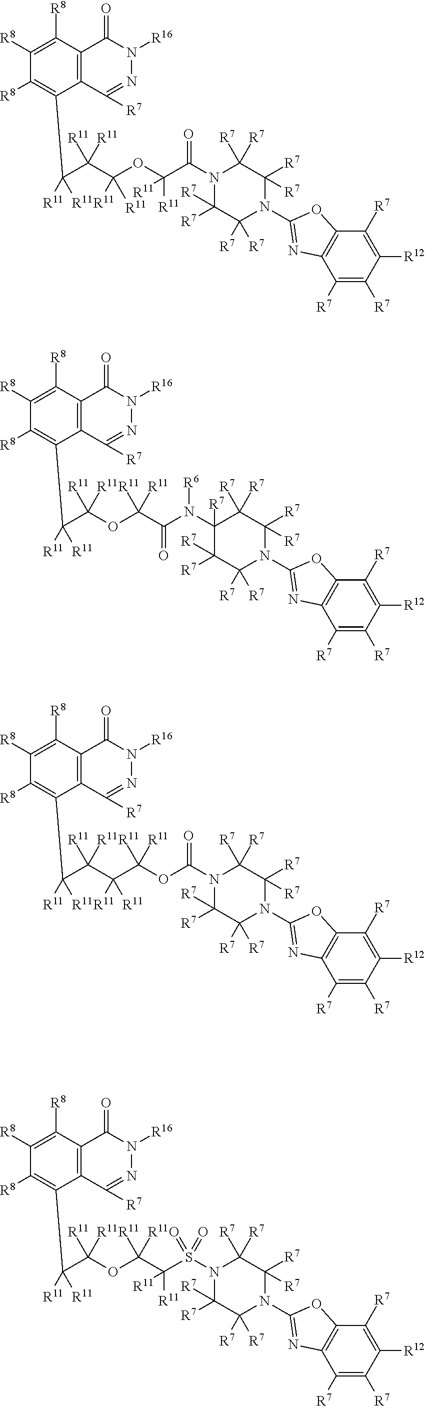

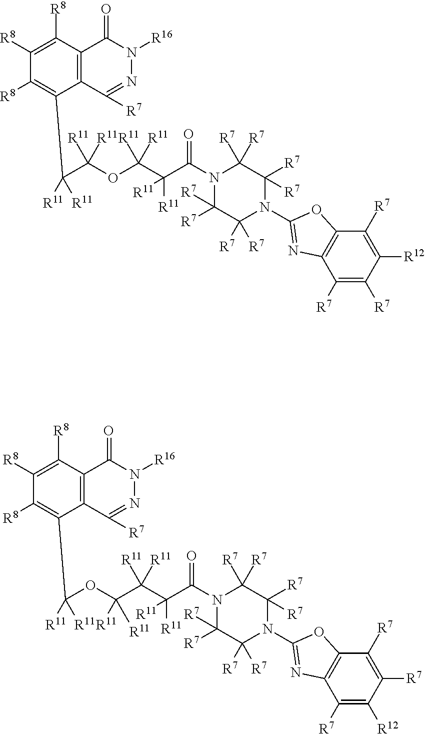

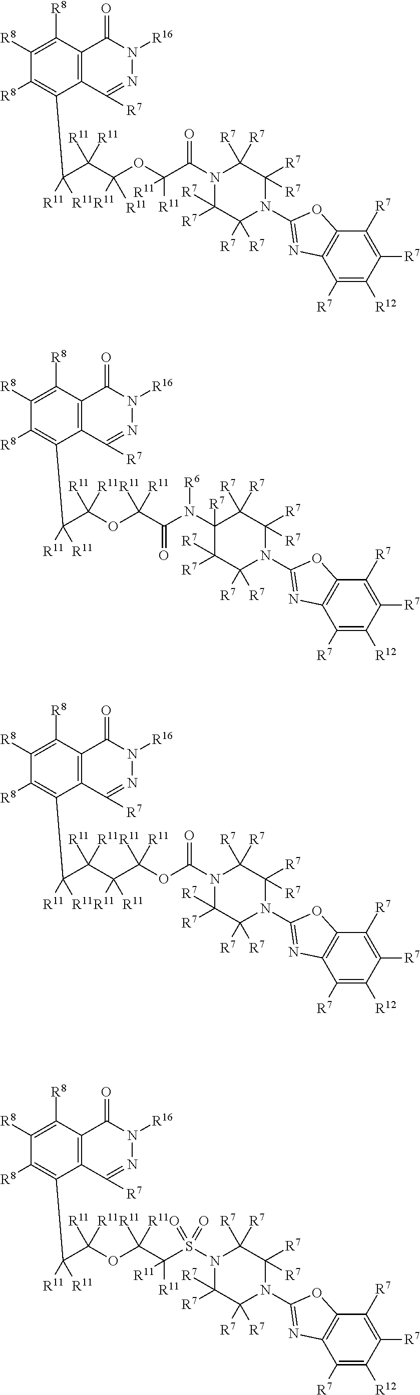

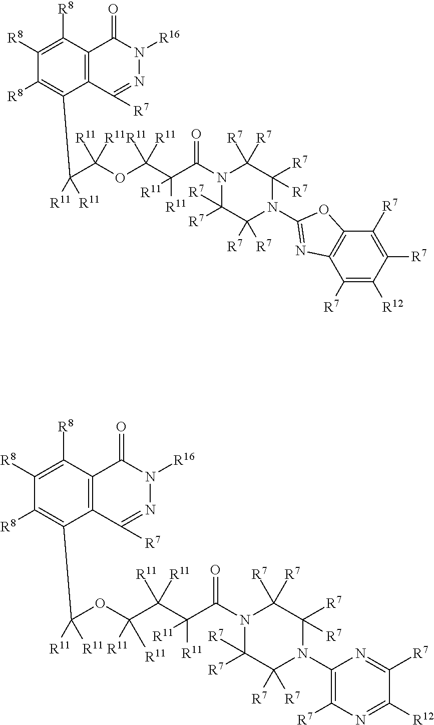

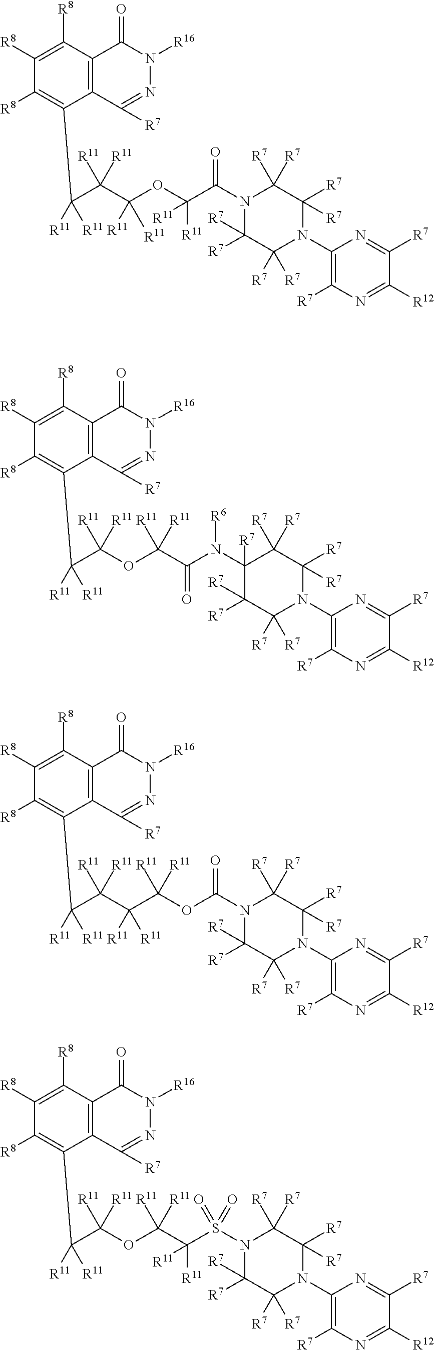

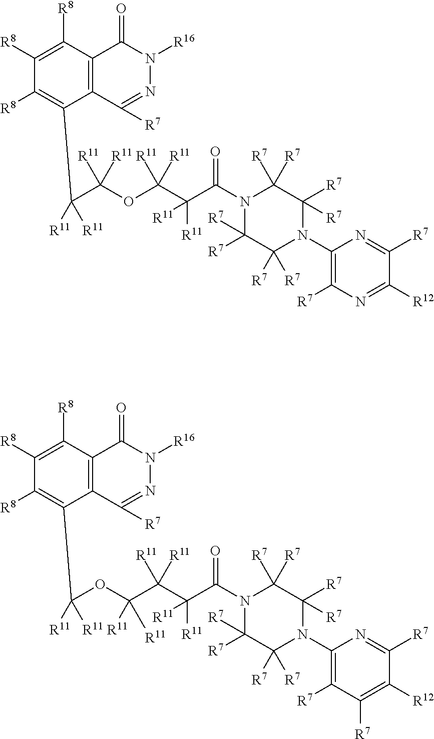

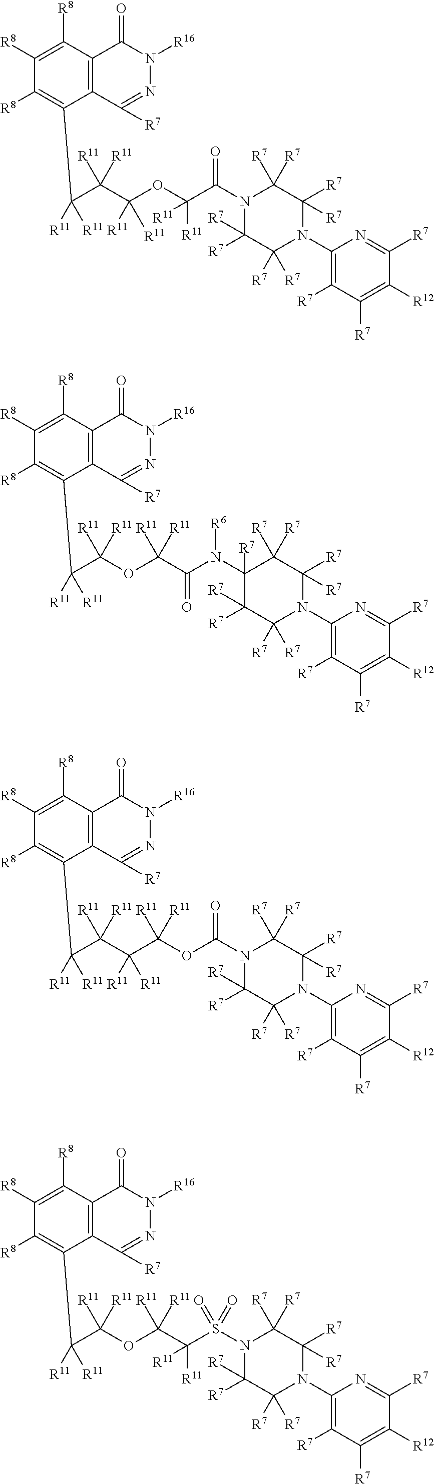

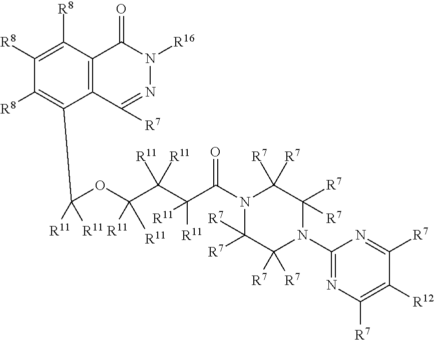

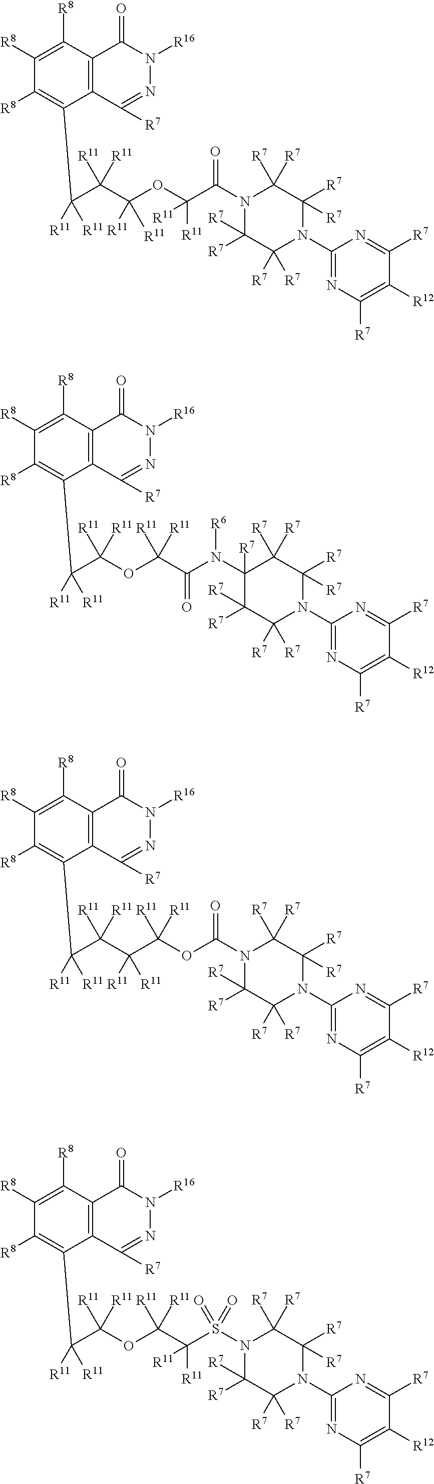

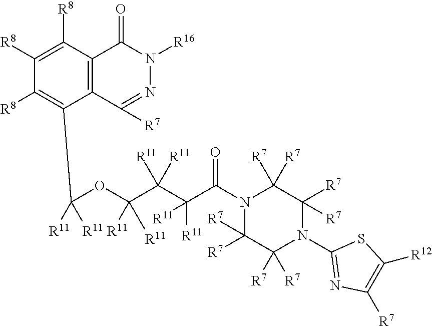

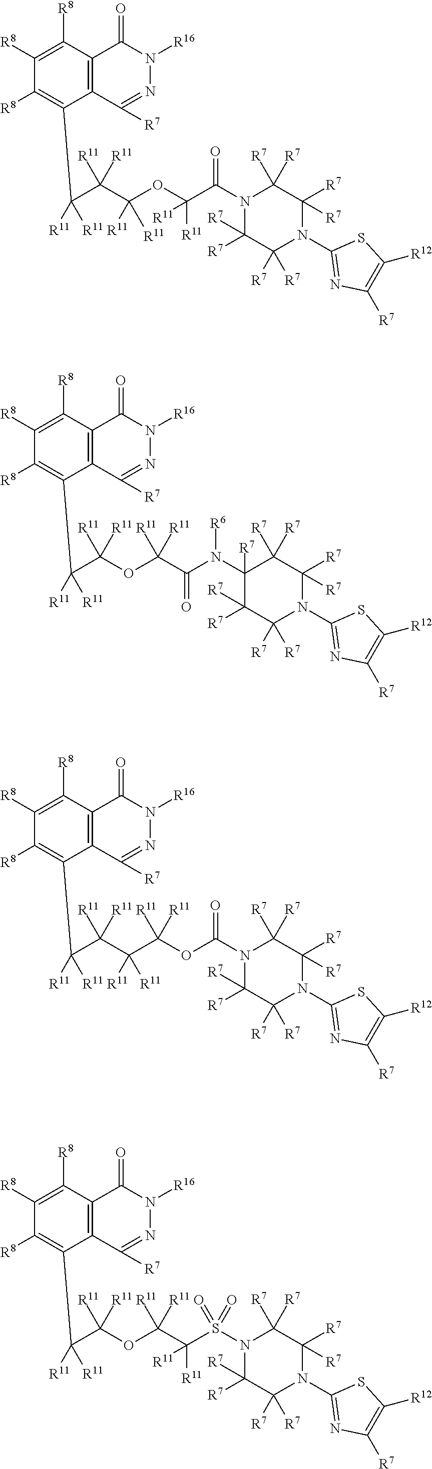

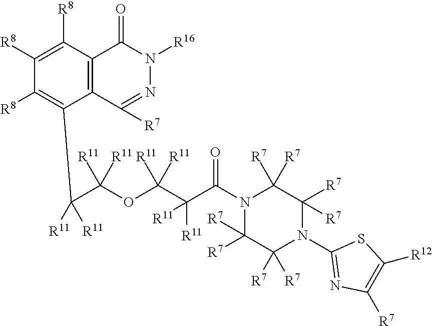

wherein R⁶, R⁷, R⁸, R¹¹, R¹² and R¹⁶ are as defined herein.

The R groups referred to in the compounds and structures herein will now be described in more detail.

As has been mentioned, the number of R substituents on an X, Y, Z or a ring atom will depend on its valency. Thus, it will be apparent in all of the embodiments of the invention, both above and below, that when an X, Y or Z, or a ring atom has three ring bonds (either 3 single bonds or a single bond and a double bond), it will have no substituents if it is N and 1 substituent (H or an organic group as defined herein) if it is C, and when X, Y or Z, or a ring atom has two ring bonds (2 single bonds), it will have 1 substituent (H or an organic group as defined herein) if it is N and 2 substituents if it is C (each independently chosen from H or an organic group as defined herein). Of course, if X or Z is O there will not be any substituents. If X or Z is S it may have no substituents, or it may be a sulphonyl group.

As has been mentioned, in all of the embodiments of this invention (both above and below herein), the substituent is not especially limited, provided that it does not prevent the PARP7 inhibitory function from occurring. However, in typical embodiments, the substituents may be selected independently as follows.

R⁵, R⁷, R⁸, R¹¹ and R¹² are typically each independently selected from H and a group selected from the following groups:
deuterium
a halogen (such as —F, —Cl, —Br and —I);
a substituted or unsubstituted linear or branched C₁-C₆ alkyl group (such as Me, Et, Pr, i-Pr, n-Bu, i-Bu, t-Bu, pentyl and hexyl);
a substituted or unsubstituted linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a substituted or unsubstituted linear or branched C₁-C₆ halogenated alkyl group (such as —CH₂F, —CHF₂, —CH₂CH₂F, —CH₂Cl, —CH₂Br, —CH₂I, —CF₃, —CCl₃—CBr₃, —Cl₃, —CH₂CF₃, —CH₂CCl₃, —CH₂CBr₃, and —CH₂Cl₃);
—NH₂ or a substituted or unsubstituted linear or branched primary secondary or tertiary C₁-C₆ amine group (such as —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a substituted or unsubstituted amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2, (3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph,
a substituted or unsubstituted cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a substituted or unsubstituted cyclic C₃-C₈ alkyl group (such as cyclopropyl, cyclobutyl, cyclopentyl, cyclohexyl, cycloheptyl and cyclooctyl);
an —OH group or a substituted or unsubstituted linear or branched C₁-C₆ alcohol group (such as —CH₂OH, —CH₂CH₂OH, —CH(CH₃)CH₂OH, —C(CH₃)₂OH, —CH₂CH₂CH₂OH, —CH₂CH₂CH₂CH₂OH, —CH(CH₃)CH₂CH₂OH, —CH(CH₃)CH(CH₃)OH, —CH(CH₂CH₃)CH₂OH, —C(CH₃)₂CH₂OH, —CH₂CH₂CH₂CH₂CH₂OH, and —CH₂CH₂CH₂CH₂CH₂CH₂OH); —a substituted or unsubstituted linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a substituted or unsubstituted linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a substituted or unsubstituted linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a substituted or unsubstituted linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a substituted or unsubstituted linear or branched C₁-C₇ amino carbonyl group (such as —NH—CO-Me, —NH—CO-Et, —NH—CO—Pr, —NH—CO-Bu, —NH—CO-pentyl, —NH—CO-hexyl, —NH—CO-Ph, —NMe-CO-Me, —NMe-CO-Et, —NMe-CO—Pr, —NMe-CO-Bu, —NMe-CO-pentyl, —NMe-CO-hexyl, —NMe-CO-Ph;
a substituted or unsubstituted linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a substituted or unsubstituted linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂C H₂NHMe, —OCH₂CH₂NMe₂, —OCH₂CH₂NHEt, and —OCH₂CH₂NEt₂;
a substituted or unsubstituted sulphonyl group (such as —SO₂Me, —SO₂Et, —SO₂Pr, —SO₂iPr, —SO₂Ph, —SO₂-(2,3 or 4)-F-Ph, —SO₂— cyclopropyl, —SO₂CH₂CH₂OCH₃), —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe;
a substituted or unsubstituted aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a substituted or unsubstituted aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-);
a saturated or unsaturated, substituted or unsubstituted, heterocyclic group including an aromatic heterocyclic group and/or a non-aromatic heterocyclic group (such as pyrrole-1-yl, pyrrole-2-yl, pyrrole-3-yl, pyrazole-1-yl, pyrazole-3-yl, pyrazole-4-yl, pyrazole-5-yl, imidazole-1-yl, imidazole-2-yl, imidazole-4-yl, imidazole-5-yl, 1,2,3-triazole-1-yl, 1,2,3-triazole-4-yl, 1,2,3-triazole-5-yl, 1,2,4-triazole-1-yl, 1,2,4-triazole-3-yl, 1,2,4-triazole-5-yl, pyridin-2-yl, pyridin-3-yl, pyridin-4-yl, pyridazine-3-yl, pyridazine-4-yl, pyrimidin-2-yl, pyrimidin-4-yl, pyrimidin-5-yl, pyrimidin-6-yl, pyrazine-2-yl, pyrrolidine-1-yl, pyrrolidine-2-yl, pyrrolidine-3-yl, piperidine-1-yl, piperidine-2-yl, piperidine-3-yl, piperidine-4-yl, 2-azapiperidine-1-yl, 2-azapiperidine-3-yl, 2-azapiperidine-4-yl, 3-azapiperidine-1-yl, 3-azapiperidine-2-yl, 3-azapiperidine-4-yl, 3-azapiperidine-5-yl, piperazine-1-yl, piperazine-2-yl, furan-2-yl, furan-3-yl, pyran-2-yl, pyran-3-yl, pyran-4-yl, 2-azapyran-2-yl, 2-azapyran-3-yl, 2-azapyran-4-yl, 2-azapyran-5-yl, 2-azapyran-6-yl, 3-azapyran-2-yl, 3-azapyran-4-yl, 3-azapyran-5-yl, 3-azapyran-6-yl, 4-azapyran-2-yl, 4-azapyran-3-yl, 4-azapyran-4-yl, 4-azapyran-5-yl, 4-azapyran-6-yl, oxetan-2-yl, oxetan-3-yl, tetrahydrofuran-2-yl, tetrahydrofuran-3-yl, 2-aza-tetrahydrofuran-2-yl, 2-aza-tetrahydrofuran-3-yl, 2-aza-tetrahydrofuran-4-yl, 2-aza-tetrahydrofuran-5-yl, 3-aza-tetrahydrofuran-2-yl, 3-aza-tetrahydrofuran-3-yl, 3-aza-tetrahydrofuran-4-yl, 3-aza-tetrahydrofuran-5-yl, tetrahydropyran-2-yl, tetrahydropyran-3-yl, tetrahydropyran-4-yl, 2-aza-tetrahydropyran-2-yl, 2-aza-tetrahydropyran-3-yl, 2-aza-tetrahydropyran-4-yl, 2-aza-tetrahydropyran-5-yl, 2-aza-tetrahydropyran-6-yl, 3-aza-tetrahydropyran-2-yl, 3-aza-tetrahydropyran-3-yl, 3-aza-tetrahydropyran-4-yl, 3-aza-tetrahydropyran-5-yl, 3-aza-tetrahydropyran-6-yl, morpholine-2-yl, morpholine-3-yl, morpholine-4-yl, thiophen-2-yl, thiophen-3-yl, isothiazole-3-yl, isothiazole-4-yl, isothiazole-5-yl, thiazole-2-yl, thiazole-4-yl, thiazole-5-yl, thiopyran-2-yl, thiopyran-3-yl, thiopyran-4-yl, 2-azathiopyran-2-yl, 2-azathiopyran-3-yl, 2-azathiopyran-4-yl, 2-azathiopyran-5-yl, 2-azathiopyran-6-yl, 3-azathiopyran-2-yl, 3-azathiopyran-4-yl, 3-azathiopyran-5-yl, 3-azathiopyran-6-yl, 4-azathiopyran-2-yl, 4-azathiopyran-3-yl, 4-azathiopyran-4-yl, 4-azathiopyran-5-yl, 4-azathiopyran-6-yl, thiolane-2-yl, thiolane-3-yl, thiane-2-yl, thiane-3-yl, thiane-4-yl, oxazol-2-yl, oxazol-4-yl, oxazol-5-yl, isoxazol-3-yl, isoxazol-4-yl, isoxazol-5-yl, furazan-3-yl, (1,3,4-oxadiazol)-2-yl, (1,3,4-oxadiazol)-5-yl, (1,2,4-oxadiazol)-3-yl, (1,2,4-oxadiazol)-5-yl; and tetrazole-1-yl, tetrazole-2-yl, tetrazole-5-yl); and
where there are two R groups attached to the same atom, they may together form a group which is double bonded to that atom, (such as a carbonyl group (═O) or an alkene group (═C(R′)₂) wherein each R′ group is the same or different and is H or an organic group, preferably H or a straight or branched C₁-C₆ alkyl group).
   R⁷ and R⁸ may also be independently selected from a nitrile group.

More typically, R⁵ is independently selected from H, deuterium, a halogen (such as —F, —Cl, —Br, and —I, preferably F), a substituted or unsubstituted C₁-C₆ alkyl group, a substituted or unsubstituted linear or branched C₁-C₆ halogenated alkyl group, an —OH group or a substituted or unsubstituted linear or branched C₁-C₆ alcohol group, an —NH₂ group or a substituted or unsubstituted C₁-C₆ amino group and a substituted or unsubstituted C₁-C₆ alkoxy group; or wherein there are two R⁵ groups on the same atom which together form a carbonyl group.

More typically, R⁷ and R⁸ are each independently selected from H, deuterium, a halogen (such as —F, —Cl, —Br, and —I), a substituted or unsubstituted C₁-C₆ alkyl or cycloalkyl group, a substituted or unsubstituted linear or branched C₁-C₆ halogenated alkyl group, an —OH group or a substituted or unsubstituted linear or branched C₁-C₆ alcohol group, an —NH₂ group or a substituted or unsubstituted C₁-C₆ amino group, a substituted or unsubstituted C₁-C₆ alkoxy group, and a nitrile group; or wherein there are two R⁷ or R⁸ groups on the same atom which together form a carbonyl group.

More typically R¹¹ is selected from H, deuterium, a halogen (such as —F, —Cl, —Br, and —I, preferably —F), a substituted or unsubstituted C₁-C₆ alkyl group, a substituted or unsubstituted linear or branched C₁-C₆ halogenated alkyl group (preferably CF₃), an —NH₂ group or a substituted or unsubstituted C₁-C₆ amino group, an —OH group or a substituted or unsubstituted linear or branched C₁-C₆ alcohol group and a substituted or unsubstituted C₁-C₆ alkoxy group.

More typically, R¹² is selected from —H, —CH₃, —CN, —CF₃, —CHF₂, —CH₂F, —OCF₃, —OMe, —CH₂CF₃, —CF₂CH₃, —OCHF₂, —OCH₂F, —F, —Cl, —Br, —I, —SO₂Me, —CONHMe, t-Bu, cyclopropyl and

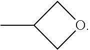

Typically, R³, R⁶, and R⁹ are each independently selected from H and a group selected from the following groups:
a substituted or unsubstituted linear or branched C₁-C₆ alkyl group (such as Me, Et, Pr, i-Pr, n-Bu, i-Bu, t-Bu, pentyl and hexyl);
a substituted or unsubstituted linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a substituted or unsubstituted linear or branched C₁-C₆ halogenated alkyl group (such as —CH₂F, —CH₂CF₃ and —CH₂CH₂F);
a substituted or unsubstituted cyclic amine or amido group (such as pyrrolidin-3-yl, piperidin-3-yl, piperidin-4-yl, 2-keto-pyrrolidinyl, 3-keto-pyrrolidinyl, 2-keto-piperidinyl, 3-keto-piperidinyl, and 4-keto-piperidinyl);
a substituted or unsubstituted cyclic C₃-C₈ alkyl group (such as cyclopropyl, cyclobutyl, cyclopentyl, cyclohexyl, cycloheptyl and cyclooctyl);
a substituted or unsubstituted linear or branched C₂-C₆ alcohol group (such as —CH₂CH₂OH, —CH(CH₃)CH₂OH, —C(CH₃)₂OH, —CH₂CH₂CH₂OH, —CH₂CH₂CH₂CH₂OH, —CH(CH₃)CH₂CH₂OH, —CH(CH₃)CH(CH₃)OH, —CH(CH₂CH₃)CH₂OH, —C(CH₃)₂CH₂OH, —CH₂CH₂CH₂CH₂CH₂OH, and —CH₂CH₂CH₂CH₂CH₂CH₂OH);
a substituted or unsubstituted linear or branched C₂-C₆ carboxylic acid group (such as —CH₂COOH, —CH₂CH₂COOH, —CH₂CH₂CH₂COOH, —CH₂CH₂CH₂CH₂COOH, and —CH₂CH₂CH₂CH₂CH₂COOH);
a substituted or unsubstituted linear or branched carbonyl group (such as —(CO)Me, —(CO)Et, —(CO)Pr, —(CO)-i-Pr, —(CO)-n-Bu, —(CO)-i-Bu, —(CO)-t-Bu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a substituted or unsubstituted linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a substituted or unsubstituted linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a substituted or unsubstituted sulphonyl group (such as —SO₂Me, —SO₂Et, —SO₂Pr, —SO₂iPr, —SO₂Ph, —SO₂-(2,3 or 4)-F-Ph, —SO₂— cyclopropyl, —SO₂CH₂CH₂OCH₃), —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe;
a substituted or unsubstituted aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a substituted or unsubstituted saturated or unsaturated, substituted or unsubstituted, heterocyclic group including an aromatic heterocyclic group and/or a non-aromatic heterocyclic group (such as pyrrole-2-yl, pyrrole-3-yl, pyrazole-3-yl, pyrazole-4-yl, pyrazole-5-yl, imidazole-2-yl, imidazole-4-yl, imidazole-5-yl, 1,2,3-triazole-4-yl, 1,2,3-triazole-5-yl, 1,2,4-triazole-3-yl, 1,2,4-triazole-5-yl, pyridin-2-yl, pyridin-3-yl, pyridin-4-yl, pyridazine-3-yl, pyridazine-4-yl, pyrimidin-2-yl, pyrimidin-4-yl, pyrimidin-5-yl, pyrimidin-6-yl, pyrazine-2-yl, pyrrolidine-2-yl, pyrrolidine-3-yl, piperidine-2-yl, piperidine-3-yl, piperidine-4-yl, 2-azapiperidine-3-yl, 2-azapiperidine-4-yl, 3-azapiperidine-2-yl, 3-azapiperidine-4-yl, 3-azapiperidine-5-yl, piperazine-2-yl, furan-2-yl, furan-3-yl, pyran-2-yl, pyran-3-yl, pyran-4-yl, 2-azapyran-3-yl, 2-azapyran-4-yl, 2-azapyran-5-yl, 2-azapyran-6-yl, 3-azapyran-2-yl, 3-azapyran-4-yl, 3-azapyran-5-yl, 3-azapyran-6-yl, 4-azapyran-2-yl, 4-azapyran-3-yl, 4-azapyran-5-yl, 4-azapyran-6-yl, oxetan-3-yl, tetrahydrofuran-2-yl, tetrahydrofuran-3-yl, 2-aza-tetrahydrofuran-3-yl, 2-aza-tetrahydrofuran-4-yl, 2-aza-tetrahydrofuran-5-yl, 3-aza-tetrahydrofuran-2-yl, 3-aza-tetrahydrofuran-4-yl, 3-aza-tetrahydrofuran-5-yl, tetrahydropyran-2-yl, tetrahydropyran-3-yl, tetrahydropyran-4-yl, 2-aza-tetrahydropyran-3-yl, 2-aza-tetrahydropyran-4-yl, 2-aza-tetrahydropyran-5-yl, 2-aza-tetrahydropyran-6-yl, 3-aza-tetrahydropyran-2-yl, 3-aza-tetrahydropyran-4-yl, 3-aza-tetrahydropyran-5-yl, 3-aza-tetrahydropyran-6-yl, morpholine-2-yl, morpholine-3-yl, thiophen-2-yl, thiophen-3-yl, isothiazole-3-yl, isothiazole-4-yl, isothiazole-5-yl, thiazole-2-yl, thiazole-4-yl, thiazole-5-yl, thiopyran-2-yl, thiopyran-3-yl, thiopyran-4-yl, 2-azathiopyran-3-yl, 2-azathiopyran-4-yl, 2-azathiopyran-5-yl, 2-azathiopyran-6-yl, 3-azathiopyran-2-yl, 3-azathiopyran-4-yl, 3-azathiopyran-5-yl, 3-azathiopyran-6-yl, 4-azathiopyran-2-yl, 4-azathiopyran-3-yl, 4-azathiopyran-5-yl, 4-azathiopyran-6-yl, thiolane-2-yl, thiolane-3-yl, thiane-2-yl, thiane-3-yl, thiane-4-yl, oxazol-2-yl, oxazol-4-yl, oxazol-5-yl, isoxazol-3-yl, isoxazol-4-yl, isoxazol-5-yl, furazan-3-yl, (1,3,4-oxadiazol)-2-yl, (1,3,4-oxadiazol)-5-yl, (1,2,4-oxadiazol)-3-yl, (1,2,4-oxadiazol)-5-yl; and tetrazole-5-yl).

More typically, R³, R⁶ and R⁹ are each independently selected from H, a substituted or unsubstituted C₁-C₆ alkyl group or a substituted or unsubstituted linear or branched C₁-C₆ halogenated alkyl group.

Preferably, R¹⁶ is absent or selected from H, a C₁-C₃ alkyl group and a C₁-C₃ halogenated alkyl group. More preferably, R¹⁶ is H.

In some embodiments, the present invention provides a PARP7 inhibitor compound which comprises a formula selected from one of the following:

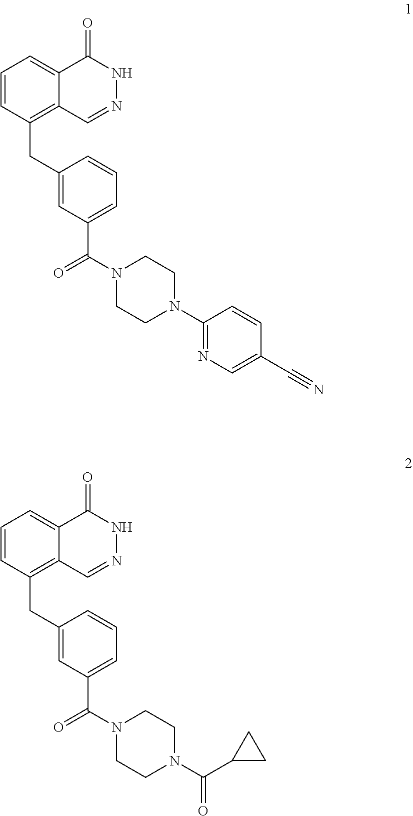 [SMILES: N#Cc1ccc(N2CCN(C(=O)c3cccc(Cc4cccc5c(=O)[nH]ncc45)c3)CC2)nc1 ; O=C(c1cccc(Cc2cccc3c(=O)[nH]ncc23)c1)N1CCN(C(=O)C2CC2)CC1]

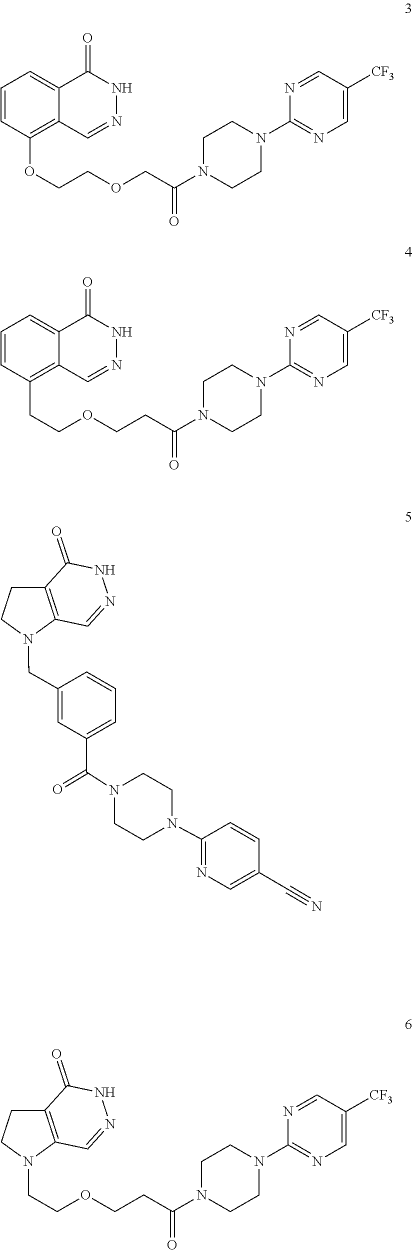 [SMILES: O=C(COCCOc1cccc2c(=O)[nH]ncc12)N1CCN(c2ncc(C(F)(F)F)cn2)CC1 ; O=C(CCOCCc1cccc2c(=O)[nH]ncc12)N1CCN(c2ncc(C(F)(F)F)cn2)CC1 ; N#Cc1ccc(N2CCN(C(=O)c3cccc(CN4CCc5c4cn[nH]c5=O)c3)CC2)nc1 ; O=C(CCOCCN1CCc2c1cn[nH]c2=O)N1CCN(c2ncc(C(F)(F)F)cn2)CC1]

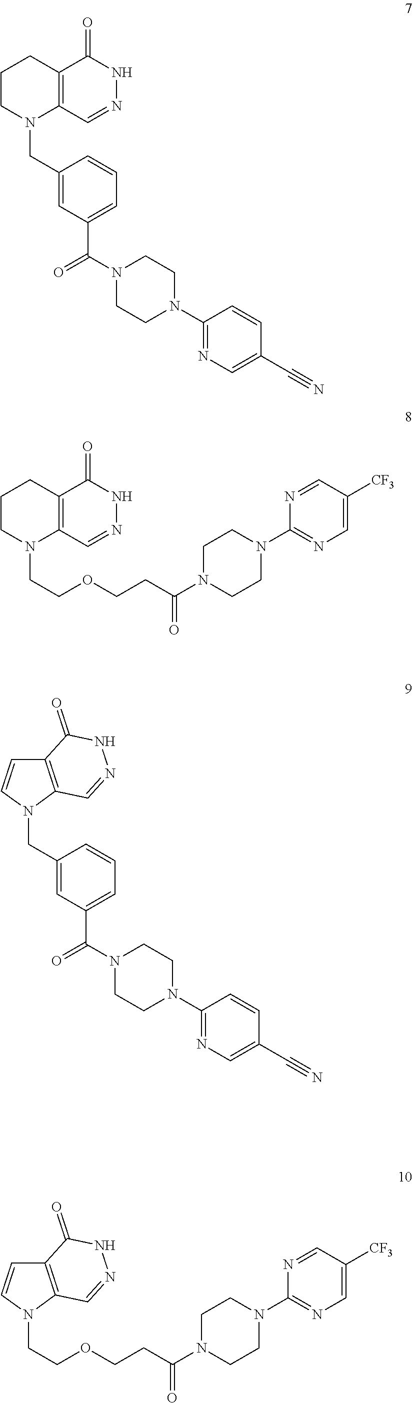 [SMILES: N#Cc1ccc(N2CCN(C(=O)c3cccc(CN4CCCc5c4cn[nH]c5=O)c3)CC2)nc1 ; O=C(CCOCCN1CCCc2c1cn[nH]c2=O)N1CCN(c2ncc(C(F)(F)F)cn2)CC1 ; N#Cc1ccc(N2CCN(C(=O)c3cccc(Cn4ccc5c(=O)[nH]ncc54)c3)CC2)nc1 ; O=C(CCOCCn1ccc2c(=O)[nH]ncc21)N1CCN(c2ncc(C(F)(F)F)cn2)CC1]

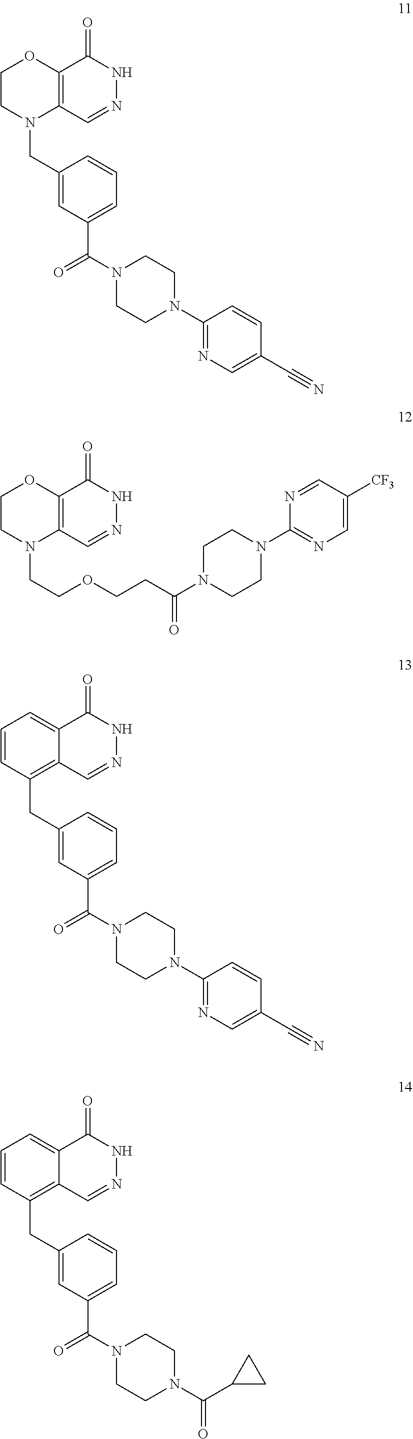 [SMILES: N#Cc1ccc(N2CCN(C(=O)c3cccc(CN4CCOc5c4cn[nH]c5=O)c3)CC2)nc1 ; O=C(CCOCCN1CCOc2c1cn[nH]c2=O)N1CCN(c2ncc(C(F)(F)F)cn2)CC1 ; N#Cc1ccc(N2CCN(C(=O)c3cccc(Cc4cccc5c(=O)[nH]ncc45)c3)CC2)nc1 ; O=C(c1cccc(Cc2cccc3c(=O)[nH]ncc23)c1)N1CCN(C(=O)C2CC2)CC1]

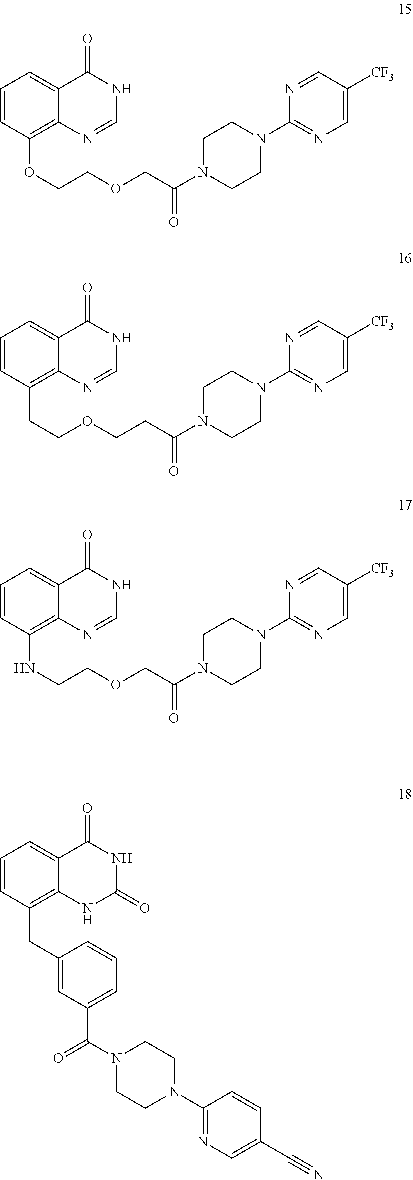 [SMILES: O=C(COCCOc1cccc2c(=O)[nH]cnc12)N1CCN(c2ncc(C(F)(F)F)cn2)CC1 ; O=C(CCOCCc1cccc2c(=O)[nH]cnc12)N1CCN(c2ncc(C(F)(F)F)cn2)CC1 ; O=C(COCCNc1cccc2c(=O)[nH]cnc12)N1CCN(c2ncc(C(F)(F)F)cn2)CC1 ; N#Cc1ccc(N2CCN(C(=O)c3cccc(Cc4cccc5c(=O)[nH]c(=O)[nH]c45)c3)CC2)nc1]

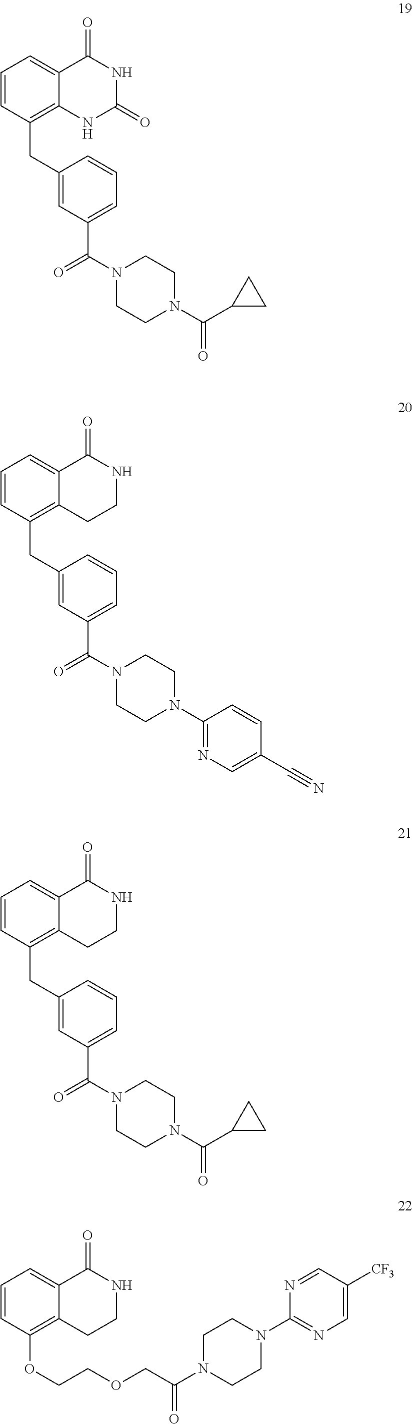 [SMILES: O=C(c1cccc(Cc2cccc3c(=O)[nH]c(=O)[nH]c23)c1)N1CCN(C(=O)C2CC2)CC1 ; N#Cc1ccc(N2CCN(C(=O)c3cccc(Cc4cccc5c4CCNC5=O)c3)CC2)nc1 ; O=C1NCCc2c(Cc3cccc(C(=O)N4CCN(C(=O)C5CC5)CC4)c3)cccc21 ; O=C1NCCc2c(OCCOCC(=O)N3CCN(c4ncc(C(F)(F)F)cn4)CC3)cccc21]

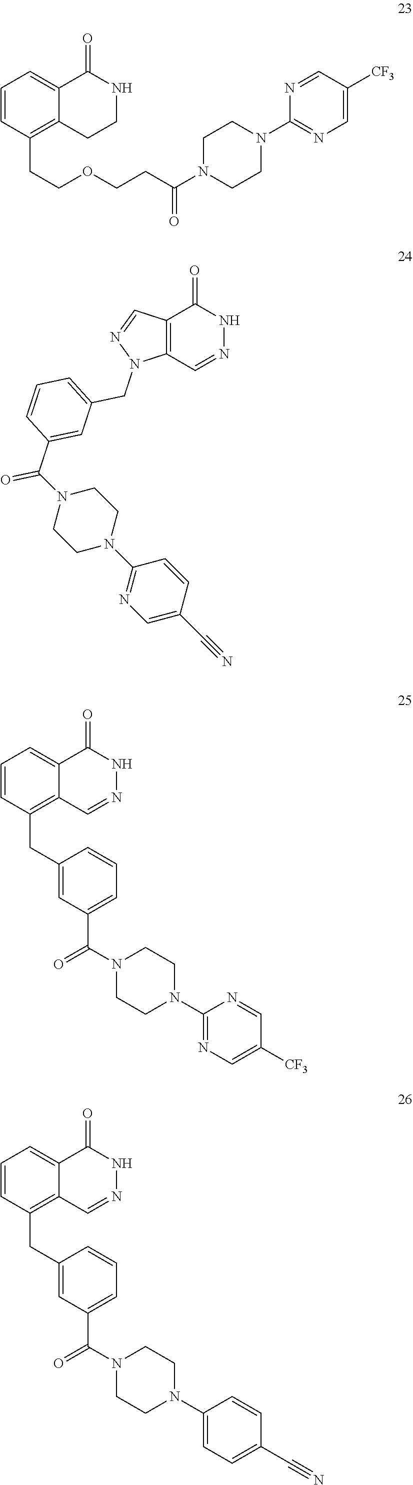 [SMILES: O=C1NCCc2c(CCOCCC(=O)N3CCN(c4ncc(C(F)(F)F)cn4)CC3)cccc21 ; N#Cc1ccc(N2CCN(C(=O)c3cccc(Cn4ncc5c(=O)[nH]ncc54)c3)CC2)nc1 ; O=C(c1cccc(Cc2cccc3c(=O)[nH]ncc23)c1)N1CCN(c2ncc(C(F)(F)F)cn2)CC1 ; N#Cc1ccc(N2CCN(C(=O)c3cccc(Cc4cccc5c(=O)[nH]ncc45)c3)CC2)cc1]

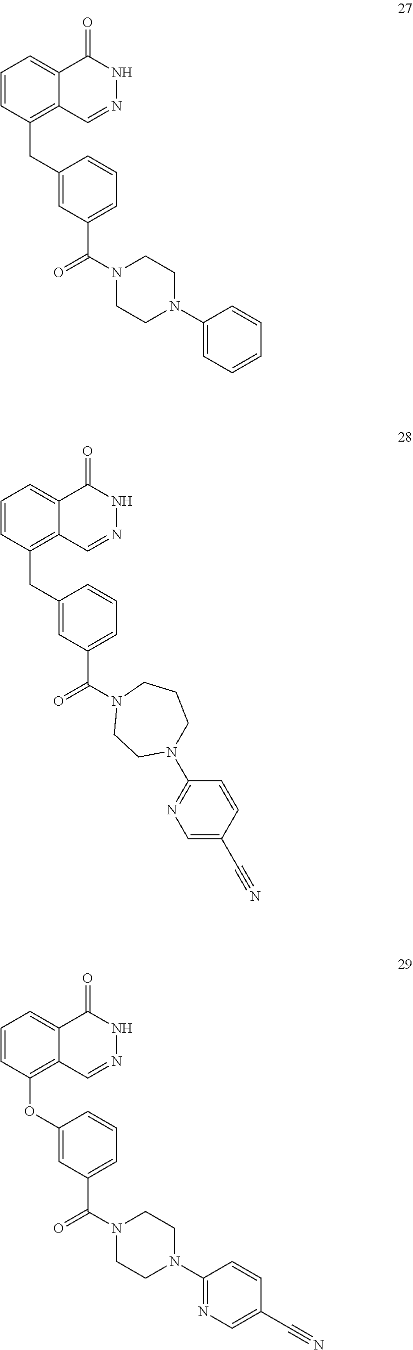 [SMILES: O=C(c1cccc(Cc2cccc3c(=O)[nH]ncc23)c1)N1CCN(c2ccccc2)CC1 ; N#Cc1ccc(N2CCCN(C(=O)c3cccc(Cc4cccc5c(=O)[nH]ncc45)c3)CC2)nc1 ; N#Cc1ccc(N2CCN(C(=O)c3cccc(Oc4cccc5c(=O)[nH]ncc45)c3)CC2)nc1]

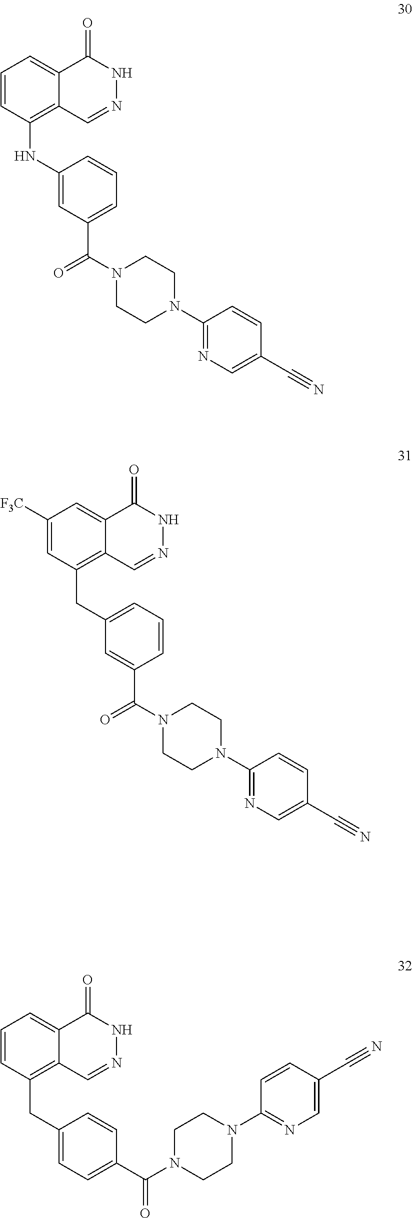 [SMILES: N#Cc1ccc(N2CCN(C(=O)c3cccc(Nc4cccc5c(=O)[nH]ncc45)c3)CC2)nc1 ; N#Cc1ccc(N2CCN(C(=O)c3cccc(Cc4cc(C(F)(F)F)cc5c(=O)[nH]ncc45)c3)CC2)nc1 ; N#Cc1ccc(N2CCN(C(=O)c3ccc(Cc4cccc5c(=O)[nH]ncc45)cc3)CC2)nc1]

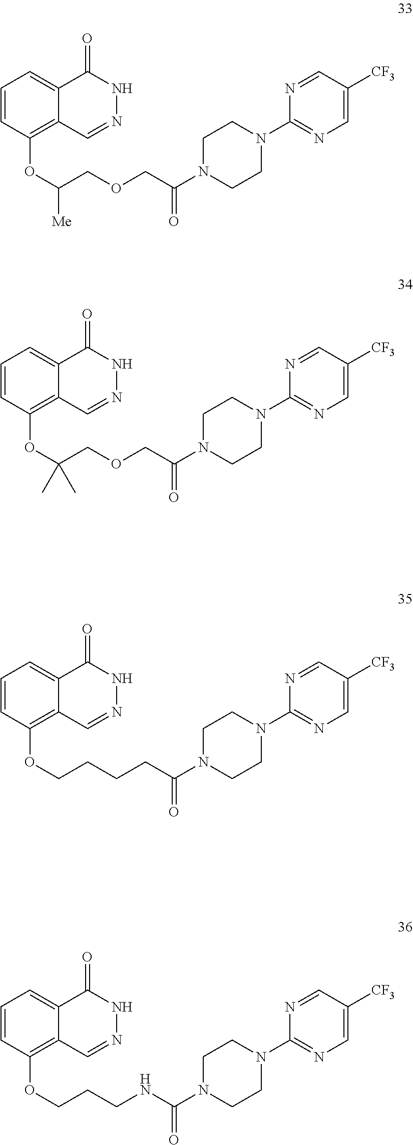 [SMILES: CC(COCC(=O)N1CCN(c2ncc(C(F)(F)F)cn2)CC1)Oc1cccc2c(=O)[nH]ncc12 ; CC(C)(COCC(=O)N1CCN(c2ncc(C(F)(F)F)cn2)CC1)Oc1cccc2c(=O)[nH]ncc12 ; O=C(CCCCOc1cccc2c(=O)[nH]ncc12)N1CCN(c2ncc(C(F)(F)F)cn2)CC1 ; O=C(NCCCOc1cccc2c(=O)[nH]ncc12)N1CCN(c2ncc(C(F)(F)F)cn2)CC1]

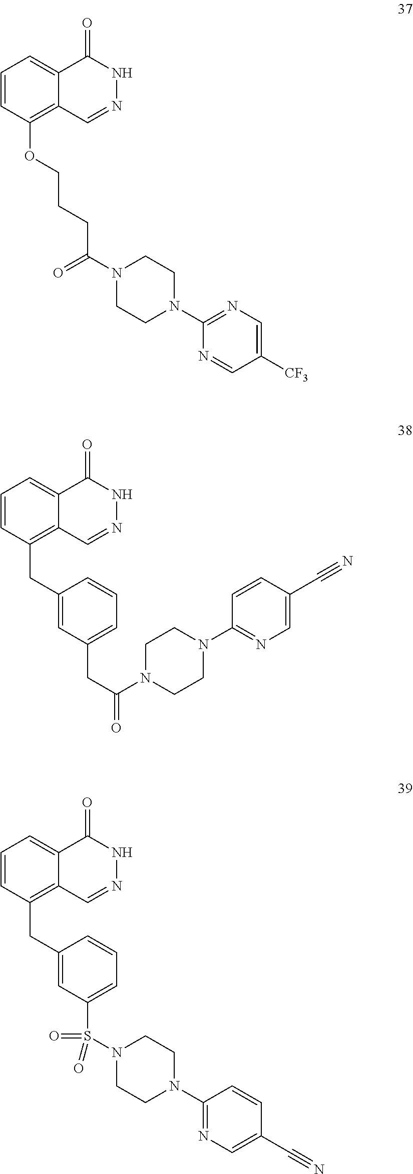 [SMILES: O=C(CCCOc1cccc2c(=O)[nH]ncc12)N1CCN(c2ncc(C(F)(F)F)cn2)CC1 ; N#Cc1ccc(N2CCN(C(=O)Cc3cccc(Cc4cccc5c(=O)[nH]ncc45)c3)CC2)nc1 ; N#Cc1ccc(N2CCN(S(=O)(=O)c3cccc(Cc4cccc5c(=O)[nH]ncc45)c3)CC2)nc1]

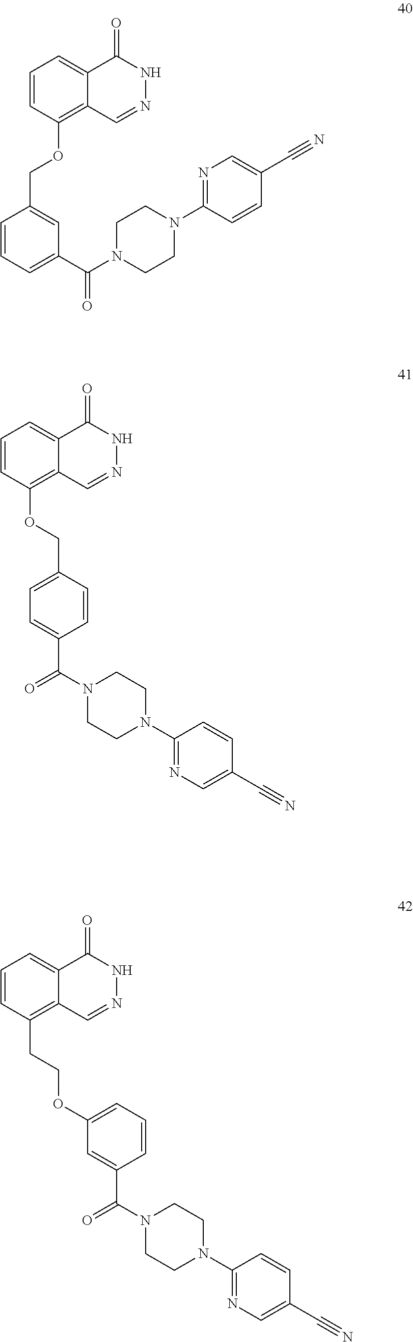 [SMILES: N#Cc1ccc(N2CCN(C(=O)c3cccc(COc4cccc5c(=O)[nH]ncc45)c3)CC2)nc1 ; N#Cc1ccc(N2CCN(C(=O)c3ccc(COc4cccc5c(=O)[nH]ncc45)cc3)CC2)nc1 ; N#Cc1ccc(N2CCN(C(=O)c3cccc(OCCc4cccc5c(=O)[nH]ncc45)c3)CC2)nc1]

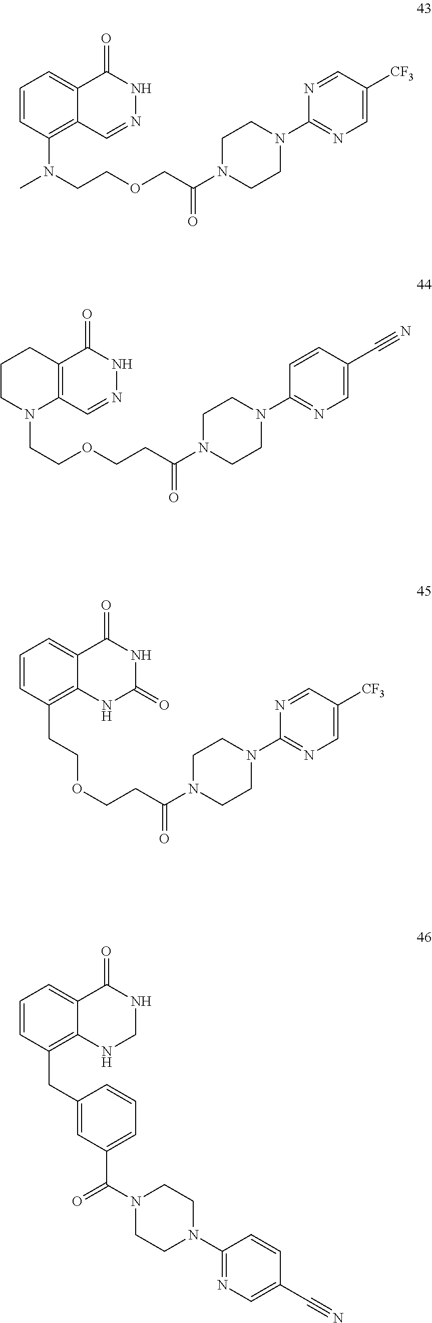 [SMILES: CN(CCOCC(=O)N1CCN(c2ncc(C(F)(F)F)cn2)CC1)c1cccc2c(=O)[nH]ncc12 ; N#Cc1ccc(N2CCN(C(=O)CCOCCN3CCCc4c3cn[nH]c4=O)CC2)nc1 ; O=C(CCOCCc1cccc2c(=O)[nH]c(=O)[nH]c12)N1CCN(c2ncc(C(F)(F)F)cn2)CC1 ; N#Cc1ccc(N2CCN(C(=O)c3cccc(Cc4cccc5c4NCNC5=O)c3)CC2)nc1]

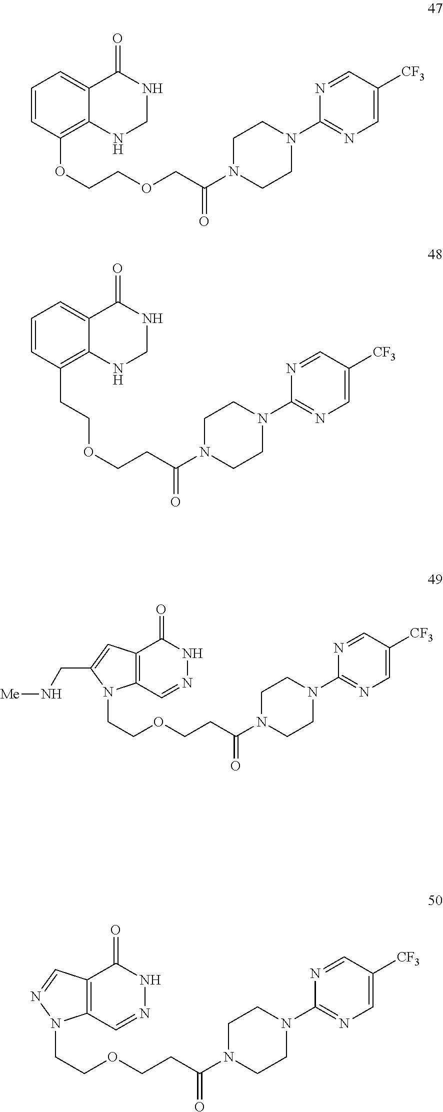 [SMILES: O=C1NCNc2c(OCCOCC(=O)N3CCN(c4ncc(C(F)(F)F)cn4)CC3)cccc21 ; O=C1NCNc2c(CCOCCC(=O)N3CCN(c4ncc(C(F)(F)F)cn4)CC3)cccc21 ; CNCc1cc2c(=O)[nH]ncc2n1CCOCCC(=O)N1CCN(c2ncc(C(F)(F)F)cn2)CC1 ; O=C(CCOCCn1ncc2c(=O)[nH]ncc21)N1CCN(c2ncc(C(F)(F)F)cn2)CC1]

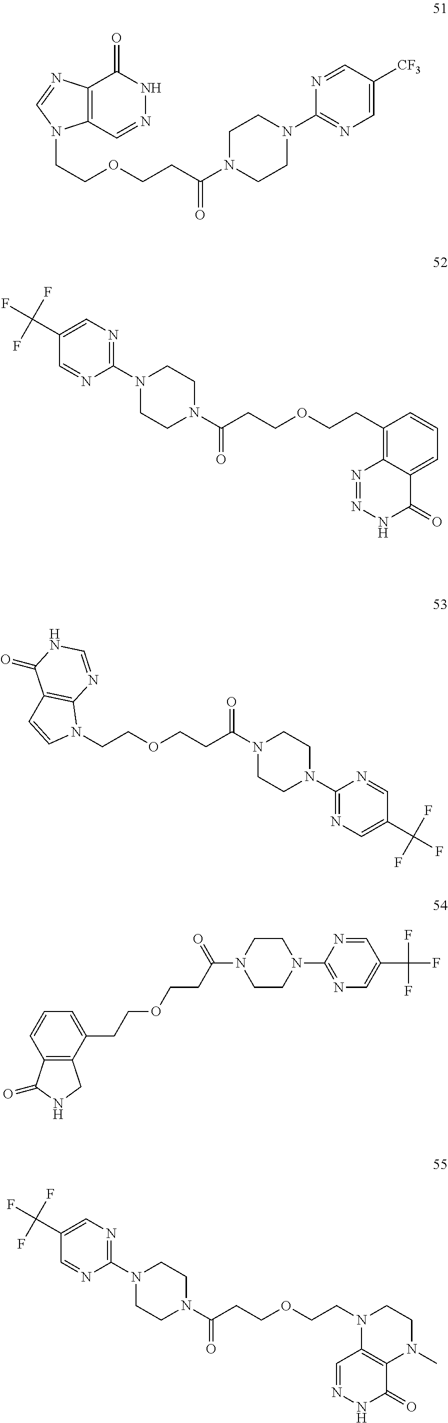 [SMILES: O=C(CCOCCn1cnc2c(=O)[nH]ncc21)N1CCN(c2ncc(C(F)(F)F)cn2)CC1 ; O=C(CCOCCc1cccc2c(=O)[nH]nnc12)N1CCN(c2ncc(C(F)(F)F)cn2)CC1 ; O=C(CCOCCn1ccc2c(=O)[nH]cnc21)N1CCN(c2ncc(C(F)(F)F)cn2)CC1 ; O=C1NCc2c(CCOCCC(=O)N3CCN(c4ncc(C(F)(F)F)cn4)CC3)cccc21 ; CN1CCN(CCOCCC(=O)N2CCN(c3ncc(C(F)(F)F)cn3)CC2)c2cn[nH]c(=O)c21]

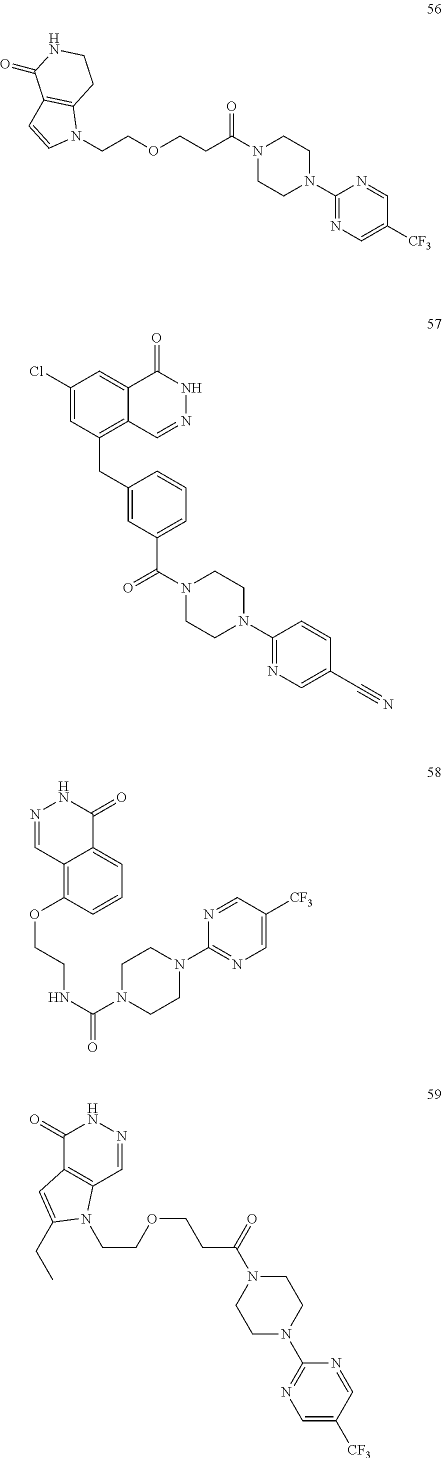 [SMILES: O=C1NCCc2c1ccn2CCOCCC(=O)N1CCN(c2ncc(C(F)(F)F)cn2)CC1 ; N#Cc1ccc(N2CCN(C(=O)c3cccc(Cc4cc(Cl)cc5c(=O)[nH]ncc45)c3)CC2)nc1 ; O=C(NCCOc1cccc2c(=O)[nH]ncc12)N1CCN(c2ncc(C(F)(F)F)cn2)CC1 ; CCc1cc2c(=O)[nH]ncc2n1CCOCCC(=O)N1CCN(c2ncc(C(F)(F)F)cn2)CC1]

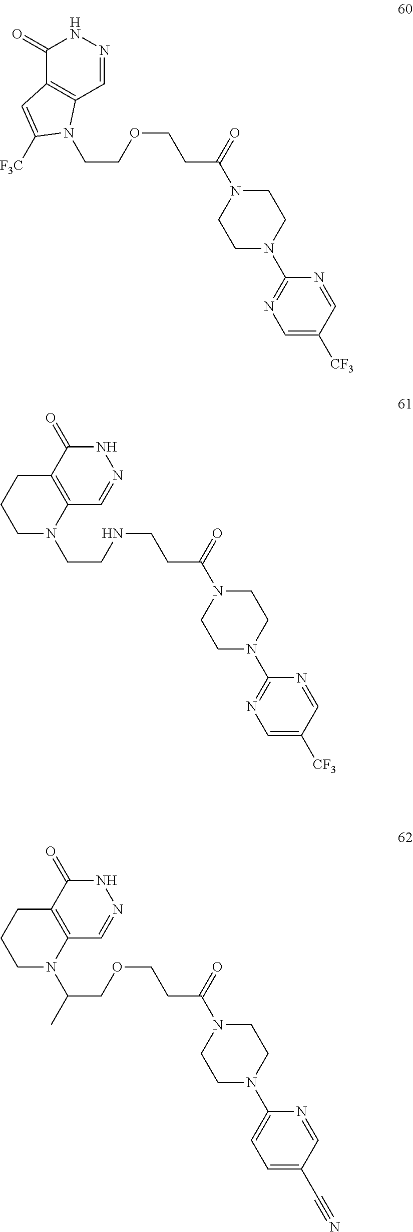 [SMILES: O=C(CCOCCn1c(C(F)(F)F)cc2c(=O)[nH]ncc21)N1CCN(c2ncc(C(F)(F)F)cn2)CC1 ; O=C(CCNCCN1CCCc2c1cn[nH]c2=O)N1CCN(c2ncc(C(F)(F)F)cn2)CC1 ; CC(COCCC(=O)N1CCN(c2ccc(C#N)cn2)CC1)N1CCCc2c1cn[nH]c2=O]

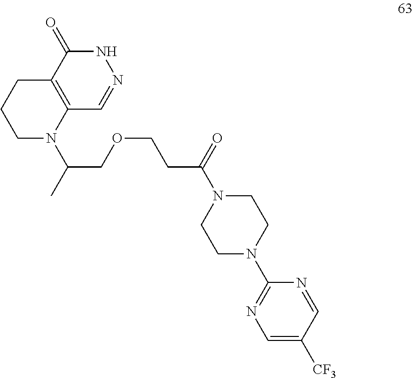 [SMILES: CC(COCCC(=O)N1CCN(c2ncc(C(F)(F)F)cn2)CC1)N1CCCc2c1cn[nH]c2=O]

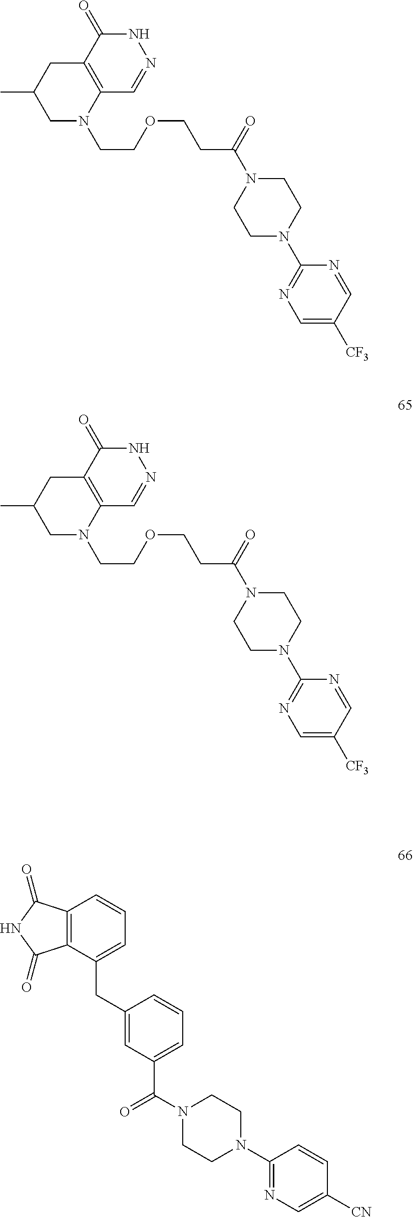 [SMILES: CC1Cc2c(cn[nH]c2=O)N(CCOCCC(=O)N2CCN(c3ncc(C(F)(F)F)cn3)CC2)C1 ; CC1Cc2c(cn[nH]c2=O)N(CCOCCC(=O)N2CCN(c3ncc(C(F)(F)F)cn3)CC2)C1 ; N#Cc1ccc(N2CCN(C(=O)c3cccc(Cc4cccc5c4C(=O)NC5=O)c3)CC2)nc1]

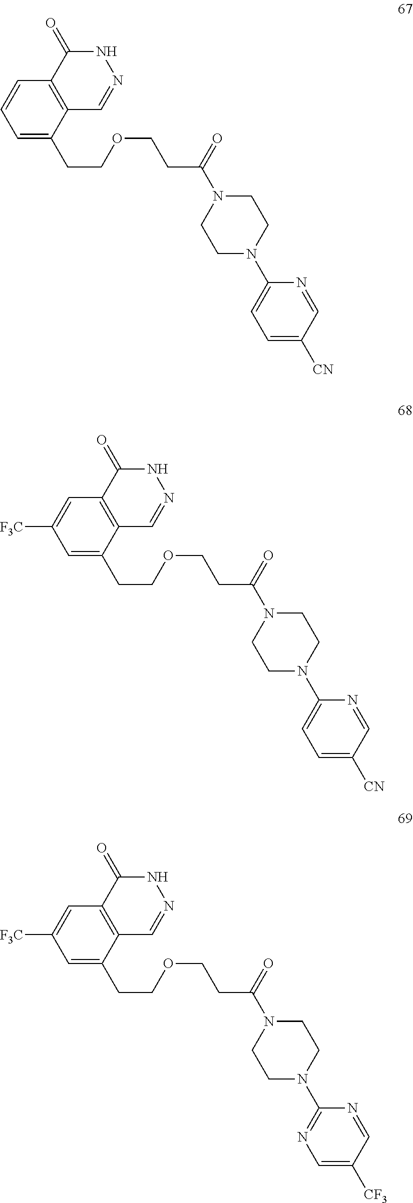 [SMILES: N#Cc1ccc(N2CCN(C(=O)CCOCCc3cccc4c(=O)[nH]ncc34)CC2)nc1 ; N#Cc1ccc(N2CCN(C(=O)CCOCCc3cc(C(F)(F)F)cc4c(=O)[nH]ncc34)CC2)nc1 ; O=C(CCOCCc1cc(C(F)(F)F)cc2c(=O)[nH]ncc12)N1CCN(c2ncc(C(F)(F)F)cn2)CC1]

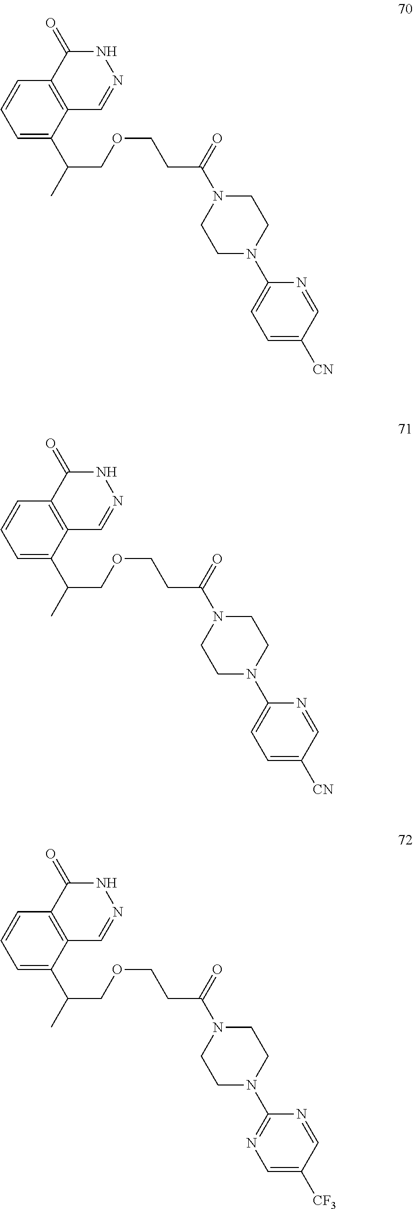 [SMILES: CC(COCCC(=O)N1CCN(c2ccc(C#N)cn2)CC1)c1cccc2c(=O)[nH]ncc12 ; CC(COCCC(=O)N1CCN(c2ccc(C#N)cn2)CC1)c1cccc2c(=O)[nH]ncc12 ; CC(COCCC(=O)N1CCN(c2ncc(C(F)(F)F)cn2)CC1)c1cccc2c(=O)[nH]ncc12]

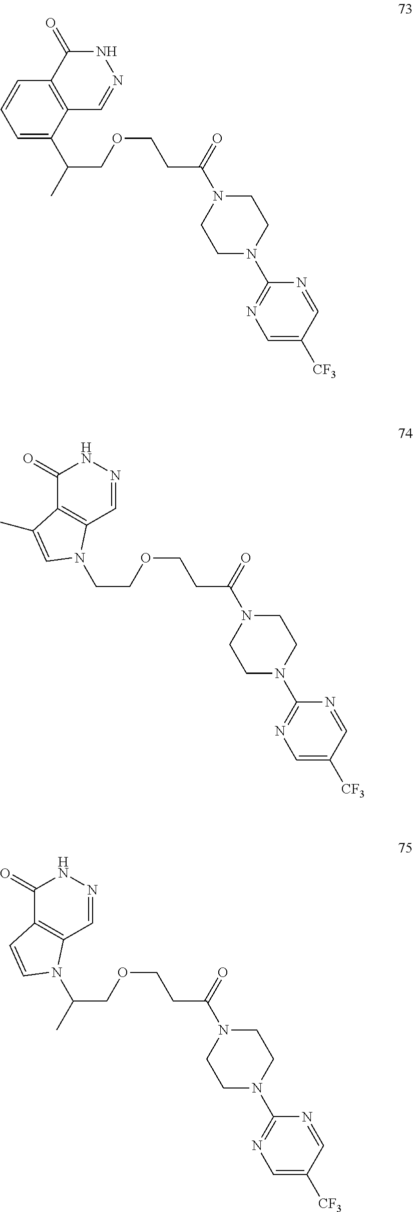 [SMILES: CC(COCCC(=O)N1CCN(c2ncc(C(F)(F)F)cn2)CC1)c1cccc2c(=O)[nH]ncc12 ; Cc1cn(CCOCCC(=O)N2CCN(c3ncc(C(F)(F)F)cn3)CC2)c2cn[nH]c(=O)c12 ; CC(COCCC(=O)N1CCN(c2ncc(C(F)(F)F)cn2)CC1)n1ccc2c(=O)[nH]ncc21]

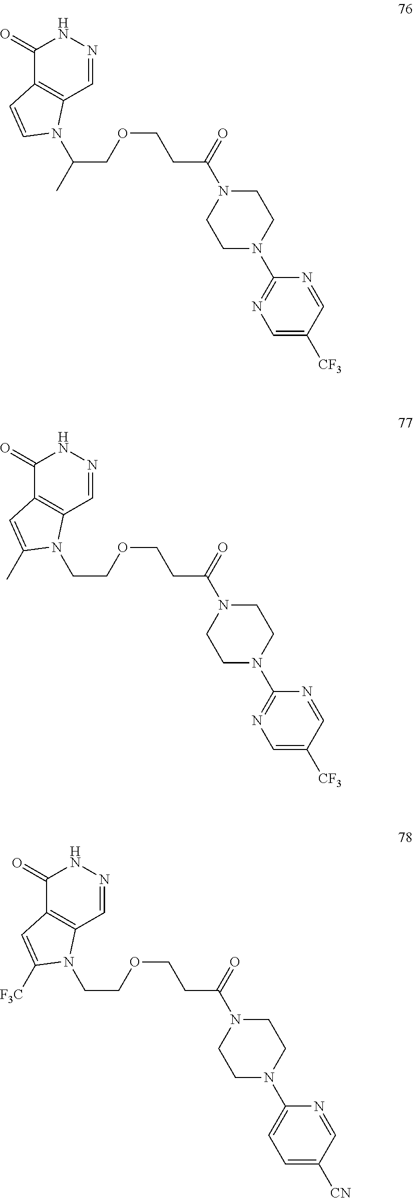 [SMILES: CC(COCCC(=O)N1CCN(c2ncc(C(F)(F)F)cn2)CC1)n1ccc2c(=O)[nH]ncc21 ; Cc1cc2c(=O)[nH]ncc2n1CCOCCC(=O)N1CCN(c2ncc(C(F)(F)F)cn2)CC1 ; N#Cc1ccc(N2CCN(C(=O)CCOCCn3c(C(F)(F)F)cc4c(=O)[nH]ncc43)CC2)nc1]

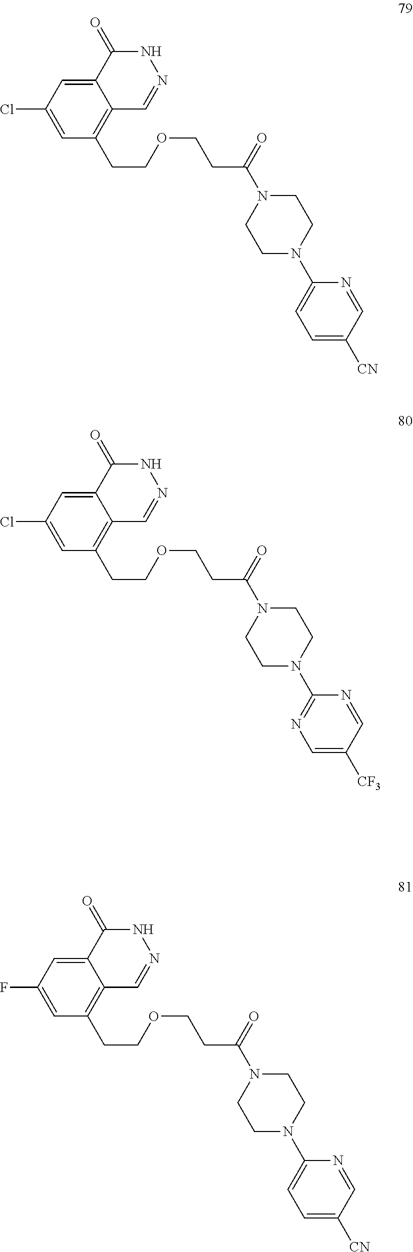 [SMILES: N#Cc1ccc(N2CCN(C(=O)CCOCCc3cc(Cl)cc4c(=O)[nH]ncc34)CC2)nc1 ; O=C(CCOCCc1cc(Cl)cc2c(=O)[nH]ncc12)N1CCN(c2ncc(C(F)(F)F)cn2)CC1 ; N#Cc1ccc(N2CCN(C(=O)CCOCCc3cc(F)cc4c(=O)[nH]ncc34)CC2)nc1]

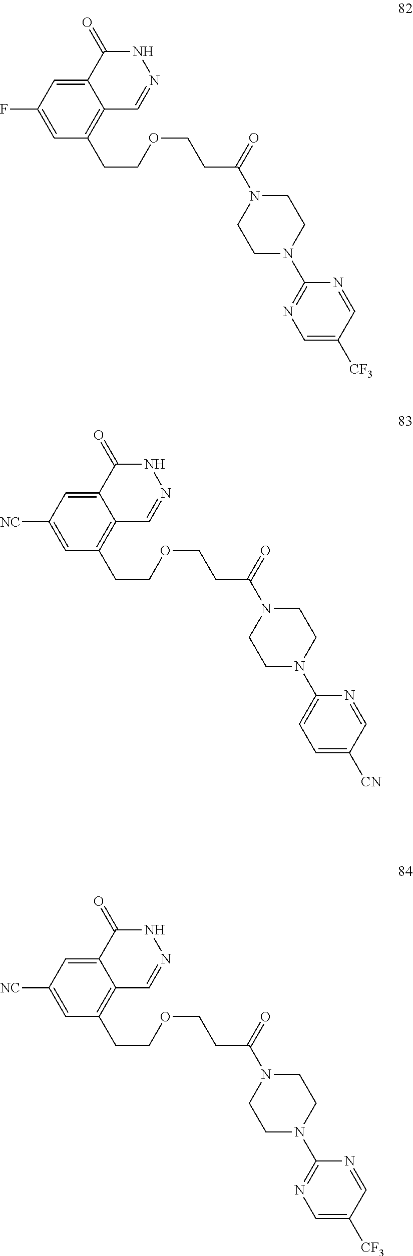 [SMILES: O=C(CCOCCc1cc(F)cc2c(=O)[nH]ncc12)N1CCN(c2ncc(C(F)(F)F)cn2)CC1 ; N#Cc1ccc(N2CCN(C(=O)CCOCCc3cc(C#N)cc4c(=O)[nH]ncc34)CC2)nc1 ; N#Cc1cc(CCOCCC(=O)N2CCN(c3ncc(C(F)(F)F)cn3)CC2)c2cn[nH]c(=O)c2c1]

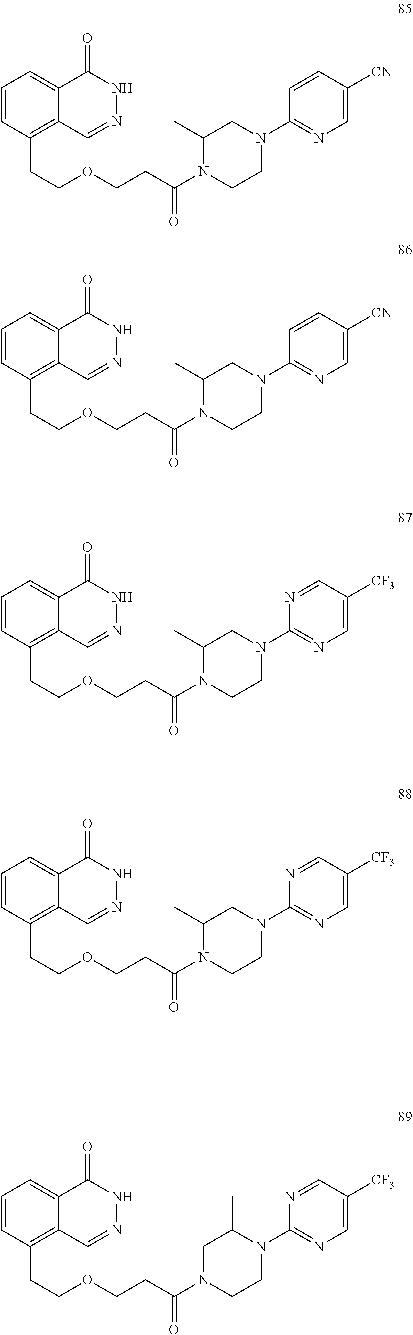 [SMILES: CC1CN(c2ccc(C#N)cn2)CCN1C(=O)CCOCCc1cccc2c(=O)[nH]ncc12 ; CC1CN(c2ccc(C#N)cn2)CCN1C(=O)CCOCCc1cccc2c(=O)[nH]ncc12 ; CC1CN(c2ncc(C(F)(F)F)cn2)CCN1C(=O)CCOCCc1cccc2c(=O)[nH]ncc12 ; CC1CN(c2ncc(C(F)(F)F)cn2)CCN1C(=O)CCOCCc1cccc2c(=O)[nH]ncc12 ; CC1CN(C(=O)CCOCCc2cccc3c(=O)[nH]ncc23)CCN1c1ncc(C(F)(F)F)cn1]

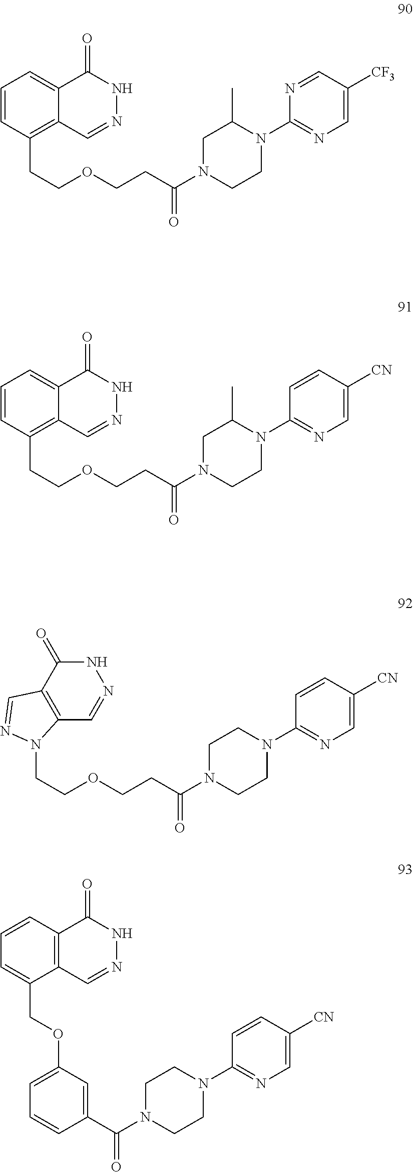 [SMILES: CC1CN(C(=O)CCOCCc2cccc3c(=O)[nH]ncc23)CCN1c1ncc(C(F)(F)F)cn1 ; CC1CN(C(=O)CCOCCc2cccc3c(=O)[nH]ncc23)CCN1c1ccc(C#N)cn1 ; N#Cc1ccc(N2CCN(C(=O)CCOCCn3ncc4c(=O)[nH]ncc43)CC2)nc1 ; N#Cc1ccc(N2CCN(C(=O)c3cccc(OCc4cccc5c(=O)[nH]ncc45)c3)CC2)nc1]

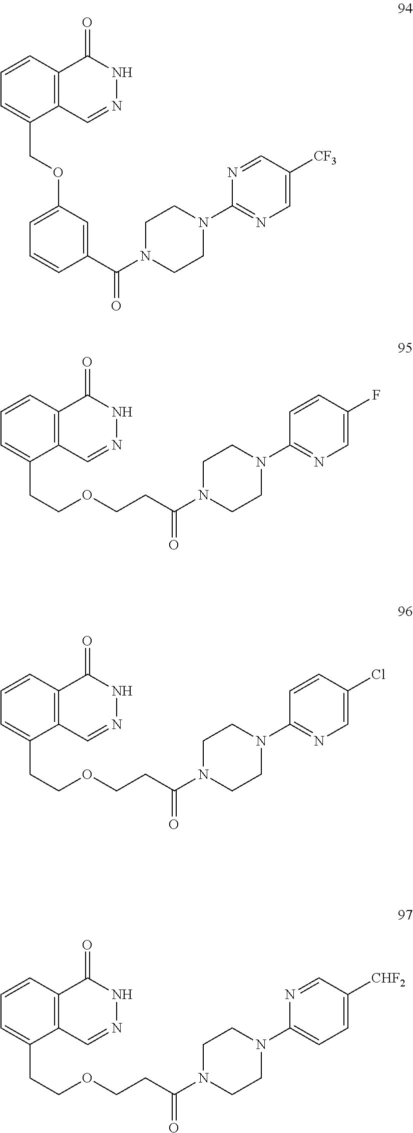 [SMILES: O=C(c1cccc(OCc2cccc3c(=O)[nH]ncc23)c1)N1CCN(c2ncc(C(F)(F)F)cn2)CC1 ; O=C(CCOCCc1cccc2c(=O)[nH]ncc12)N1CCN(c2ccc(F)cn2)CC1 ; O=C(CCOCCc1cccc2c(=O)[nH]ncc12)N1CCN(c2ccc(Cl)cn2)CC1 ; O=C(CCOCCc1cccc2c(=O)[nH]ncc12)N1CCN(c2ccc(C(F)F)cn2)CC1]

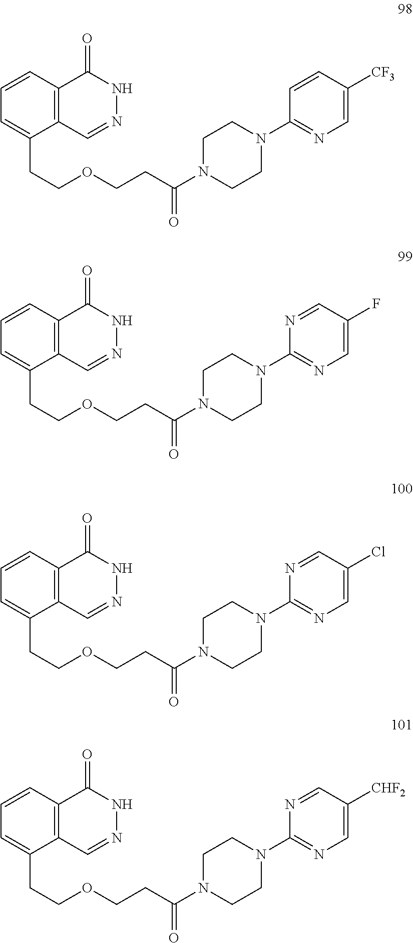 [SMILES: O=C(CCOCCc1cccc2c(=O)[nH]ncc12)N1CCN(c2ccc(C(F)(F)F)cn2)CC1 ; O=C(CCOCCc1cccc2c(=O)[nH]ncc12)N1CCN(c2ncc(F)cn2)CC1 ; O=C(CCOCCc1cccc2c(=O)[nH]ncc12)N1CCN(c2ncc(Cl)cn2)CC1 ; O=C(CCOCCc1cccc2c(=O)[nH]ncc12)N1CCN(c2ncc(C(F)F)cn2)CC1]

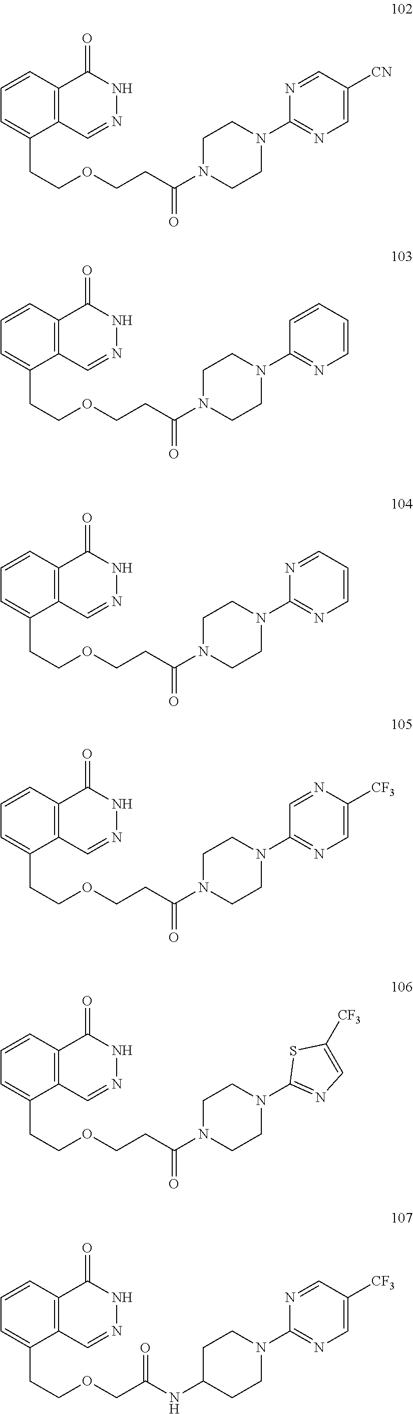 [SMILES: N#Cc1cnc(N2CCN(C(=O)CCOCCc3cccc4c(=O)[nH]ncc34)CC2)nc1 ; O=C(CCOCCc1cccc2c(=O)[nH]ncc12)N1CCN(c2ccccn2)CC1 ; O=C(CCOCCc1cccc2c(=O)[nH]ncc12)N1CCN(c2ncccn2)CC1 ; O=C(CCOCCc1cccc2c(=O)[nH]ncc12)N1CCN(c2cnc(C(F)(F)F)cn2)CC1 ; O=C(CCOCCc1cccc2c(=O)[nH]ncc12)N1CCN(c2ncc(C(F)(F)F)s2)CC1 ; O=C(COCCc1cccc2c(=O)[nH]ncc12)NC1CCN(c2ncc(C(F)(F)F)cn2)CC1]

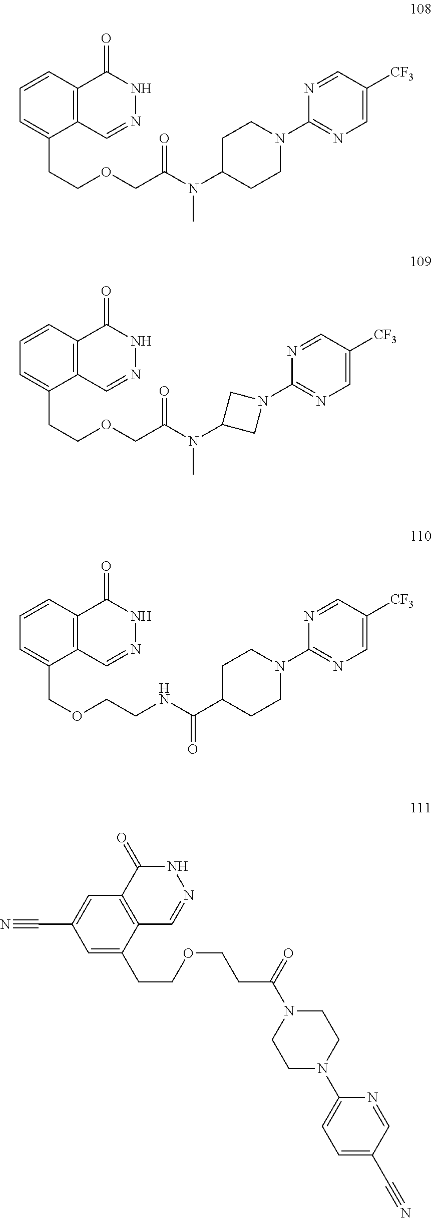 [SMILES: CN(C(=O)COCCc1cccc2c(=O)[nH]ncc12)C1CCN(c2ncc(C(F)(F)F)cn2)CC1 ; CN(C(=O)COCCc1cccc2c(=O)[nH]ncc12)C1CN(c2ncc(C(F)(F)F)cn2)C1 ; O=C(NCCOCc1cccc2c(=O)[nH]ncc12)C1CCN(c2ncc(C(F)(F)F)cn2)CC1 ; N#Cc1ccc(N2CCN(C(=O)CCOCCc3cc(C#N)cc4c(=O)[nH]ncc34)CC2)nc1]

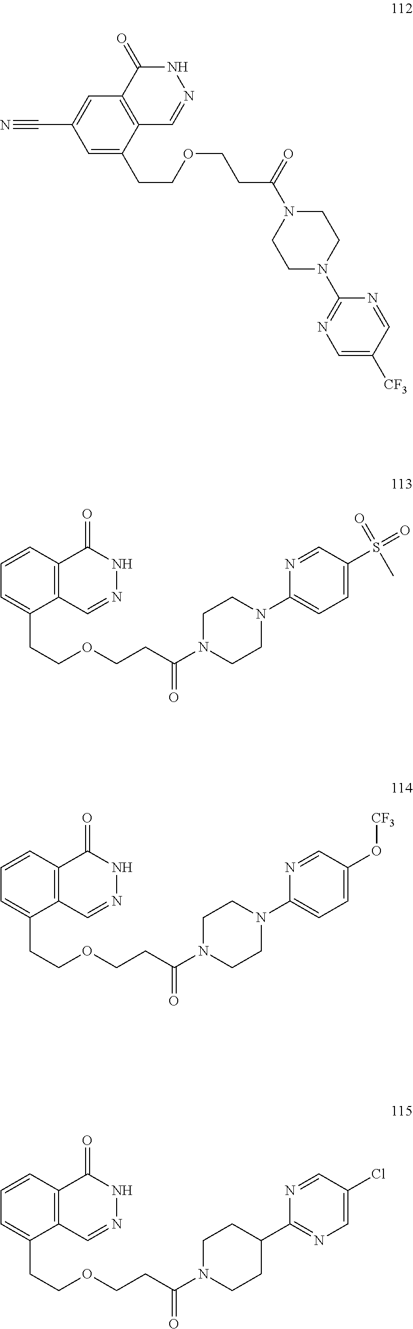 [SMILES: N#Cc1cc(CCOCCC(=O)N2CCN(c3ncc(C(F)(F)F)cn3)CC2)c2cn[nH]c(=O)c2c1 ; CS(=O)(=O)c1ccc(N2CCN(C(=O)CCOCCc3cccc4c(=O)[nH]ncc34)CC2)nc1 ; O=C(CCOCCc1cccc2c(=O)[nH]ncc12)N1CCN(c2ccc(OC(F)(F)F)cn2)CC1 ; O=C(CCOCCc1cccc2c(=O)[nH]ncc12)N1CCC(c2ncc(Cl)cn2)CC1]

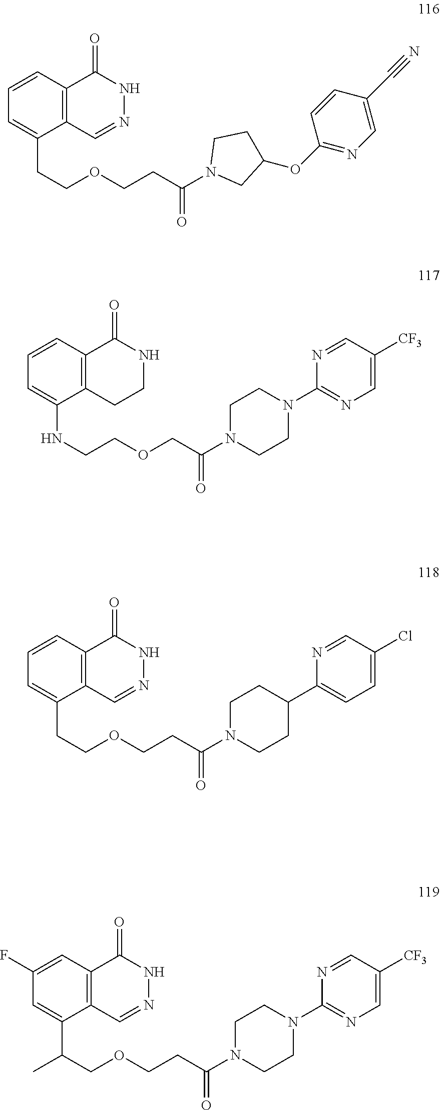 [SMILES: N#Cc1ccc(OC2CCN(C(=O)CCOCCc3cccc4c(=O)[nH]ncc34)C2)nc1 ; O=C1NCCc2c(NCCOCC(=O)N3CCN(c4ncc(C(F)(F)F)cn4)CC3)cccc21 ; O=C(CCOCCc1cccc2c(=O)[nH]ncc12)N1CCC(c2ccc(Cl)cn2)CC1 ; CC(COCCC(=O)N1CCN(c2ncc(C(F)(F)F)cn2)CC1)c1cc(F)cc2c(=O)[nH]ncc12]

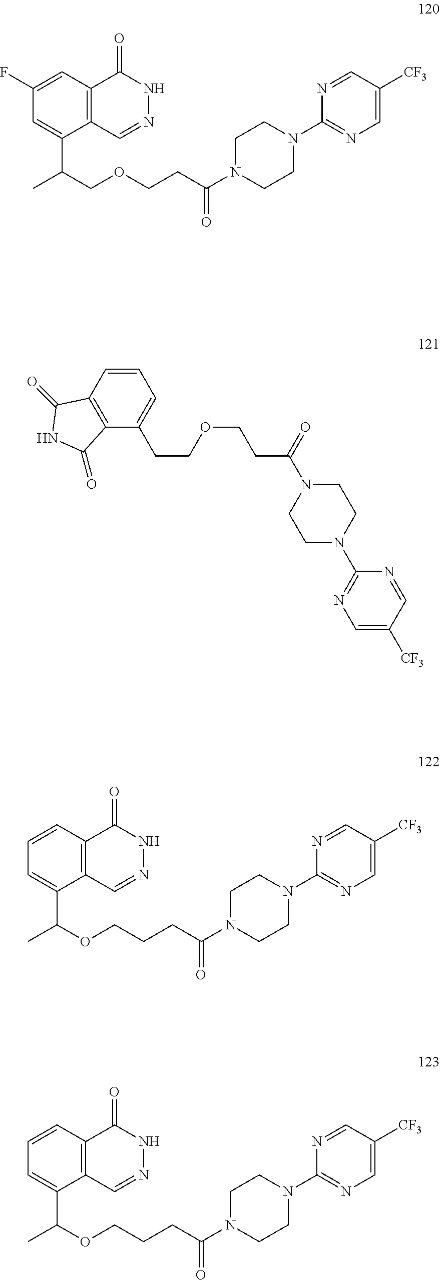 [SMILES: CC(COCCC(=O)N1CCN(c2ncc(C(F)(F)F)cn2)CC1)c1cc(F)cc2c(=O)[nH]ncc12 ; O=C1NC(=O)c2c(CCOCCC(=O)N3CCN(c4ncc(C(F)(F)F)cn4)CC3)cccc21 ; CC(OCCCC(=O)N1CCN(c2ncc(C(F)(F)F)cn2)CC1)c1cccc2c(=O)[nH]ncc12 ; CC(OCCCC(=O)N1CCN(c2ncc(C(F)(F)F)cn2)CC1)c1cccc2c(=O)[nH]ncc12]

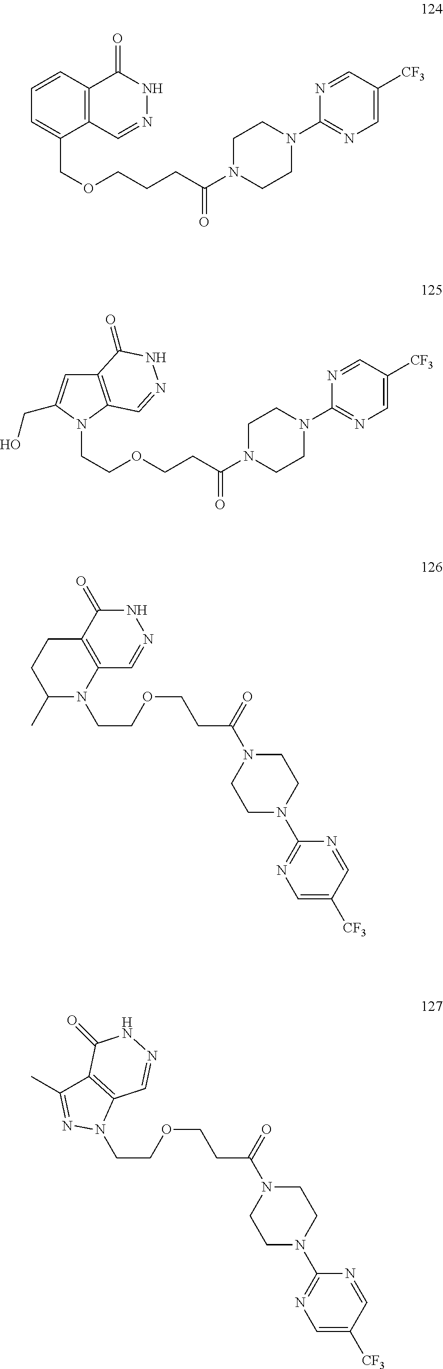 [SMILES: O=C(CCCOCc1cccc2c(=O)[nH]ncc12)N1CCN(c2ncc(C(F)(F)F)cn2)CC1 ; O=C(CCOCCn1c(CO)cc2c(=O)[nH]ncc21)N1CCN(c2ncc(C(F)(F)F)cn2)CC1 ; CC1CCc2c(cn[nH]c2=O)N1CCOCCC(=O)N1CCN(c2ncc(C(F)(F)F)cn2)CC1 ; Cc1nn(CCOCCC(=O)N2CCN(c3ncc(C(F)(F)F)cn3)CC2)c2cn[nH]c(=O)c12]

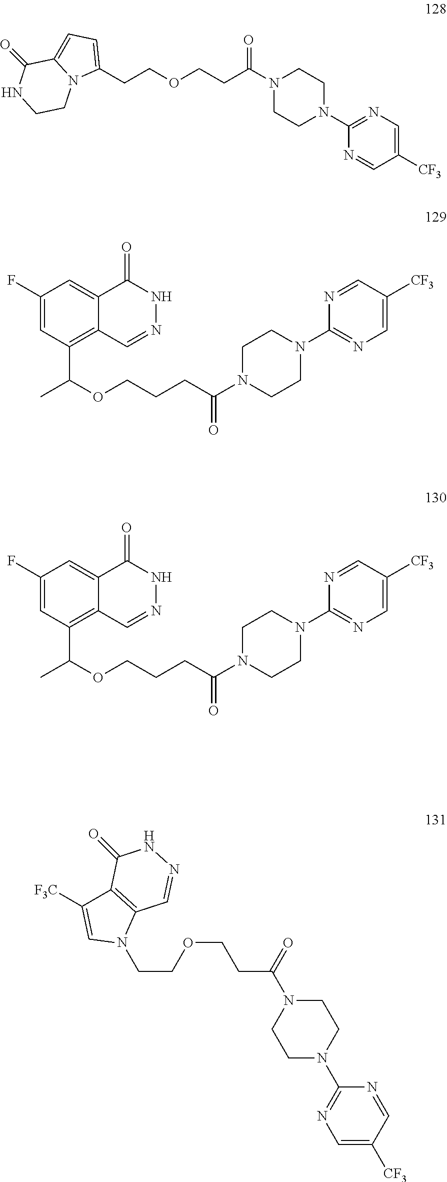 [SMILES: O=C1NCCn2c(CCOCCC(=O)N3CCN(c4ncc(C(F)(F)F)cn4)CC3)ccc21 ; CC(OCCCC(=O)N1CCN(c2ncc(C(F)(F)F)cn2)CC1)c1cc(F)cc2c(=O)[nH]ncc12 ; CC(OCCCC(=O)N1CCN(c2ncc(C(F)(F)F)cn2)CC1)c1cc(F)cc2c(=O)[nH]ncc12 ; O=C(CCOCCn1cc(C(F)(F)F)c2c(=O)[nH]ncc21)N1CCN(c2ncc(C(F)(F)F)cn2)CC1]

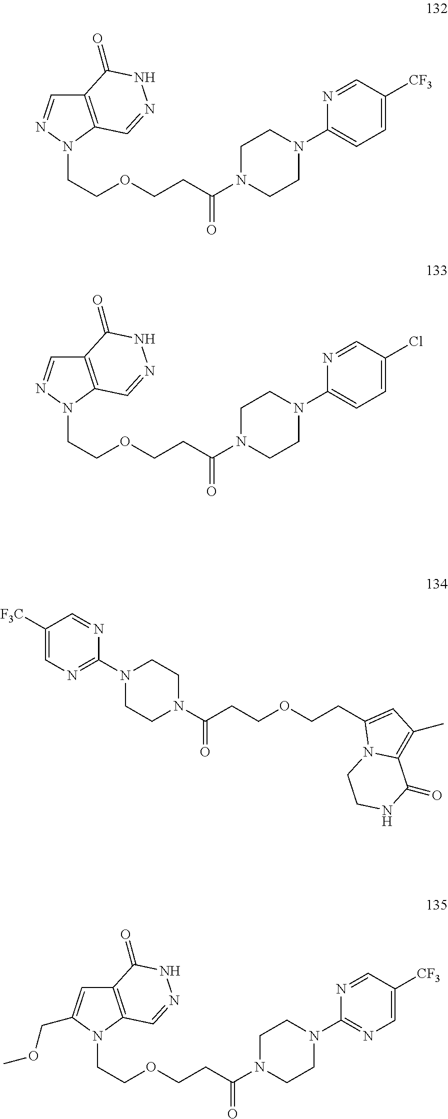

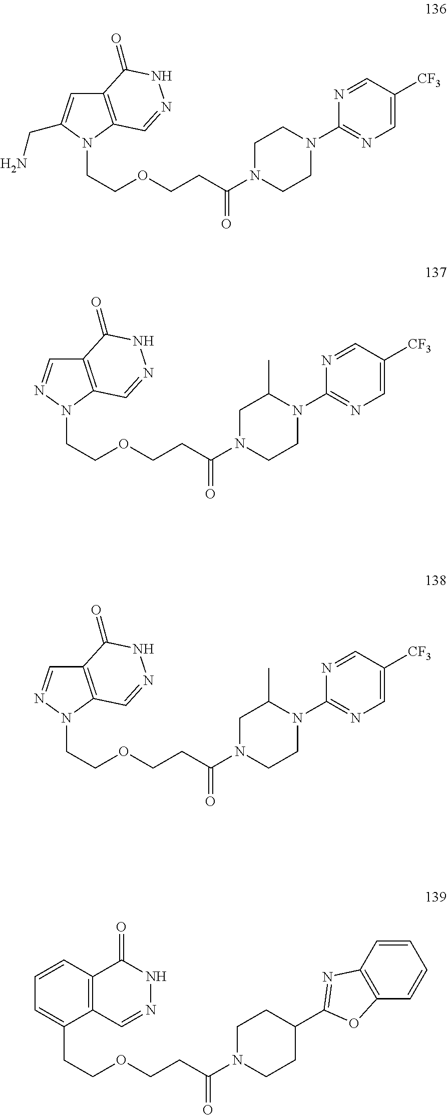 [SMILES: NCc1cc2c(=O)[nH]ncc2n1CCOCCC(=O)N1CCN(c2ncc(C(F)(F)F)cn2)CC1 ; CC1CN(C(=O)CCOCCn2ncc3c(=O)[nH]ncc32)CCN1c1ncc(C(F)(F)F)cn1 ; CC1CN(C(=O)CCOCCn2ncc3c(=O)[nH]ncc32)CCN1c1ncc(C(F)(F)F)cn1 ; O=C(CCOCCc1cccc2c(=O)[nH]ncc12)N1CCC(c2nc3ccccc3o2)CC1]

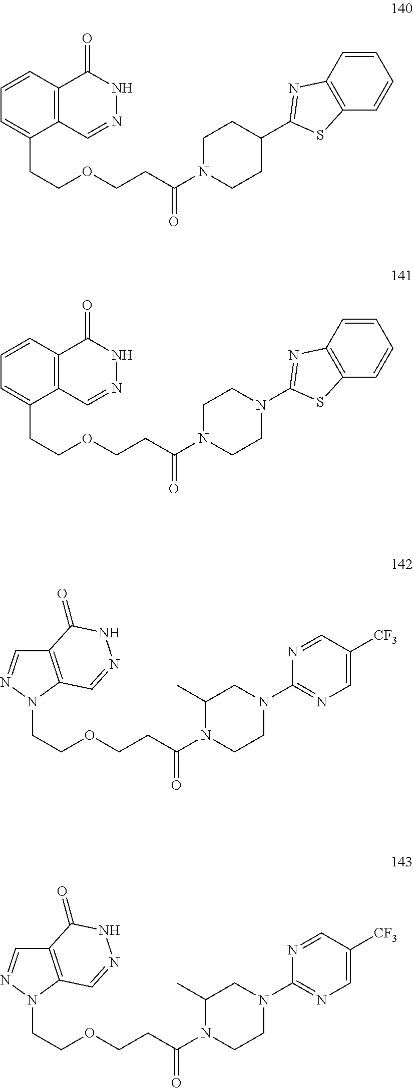 [SMILES: O=C(CCOCCc1cccc2c(=O)[nH]ncc12)N1CCC(c2nc3ccccc3s2)CC1 ; O=C(CCOCCc1cccc2c(=O)[nH]ncc12)N1CCN(c2nc3ccccc3s2)CC1 ; CC1CN(c2ncc(C(F)(F)F)cn2)CCN1C(=O)CCOCCn1ncc2c(=O)[nH]ncc21 ; CC1CN(c2ncc(C(F)(F)F)cn2)CCN1C(=O)CCOCCn1ncc2c(=O)[nH]ncc21]

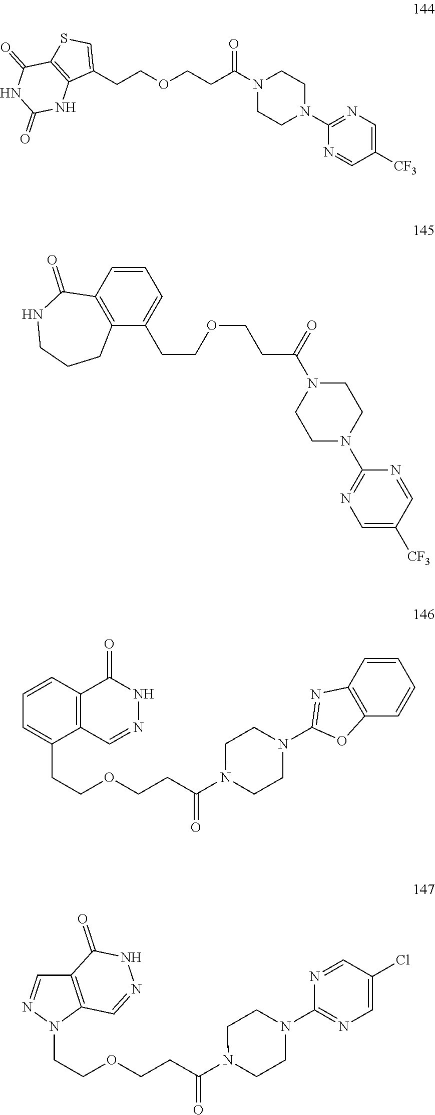 [SMILES: O=C(CCOCCc1csc2c(=O)[nH]c(=O)[nH]c12)N1CCN(c2ncc(C(F)(F)F)cn2)CC1 ; O=C1NCCCc2c(CCOCCC(=O)N3CCN(c4ncc(C(F)(F)F)cn4)CC3)cccc21 ; O=C(CCOCCc1cccc2c(=O)[nH]ncc12)N1CCN(c2nc3ccccc3o2)CC1 ; O=C(CCOCCn1ncc2c(=O)[nH]ncc21)N1CCN(c2ncc(Cl)cn2)CC1]

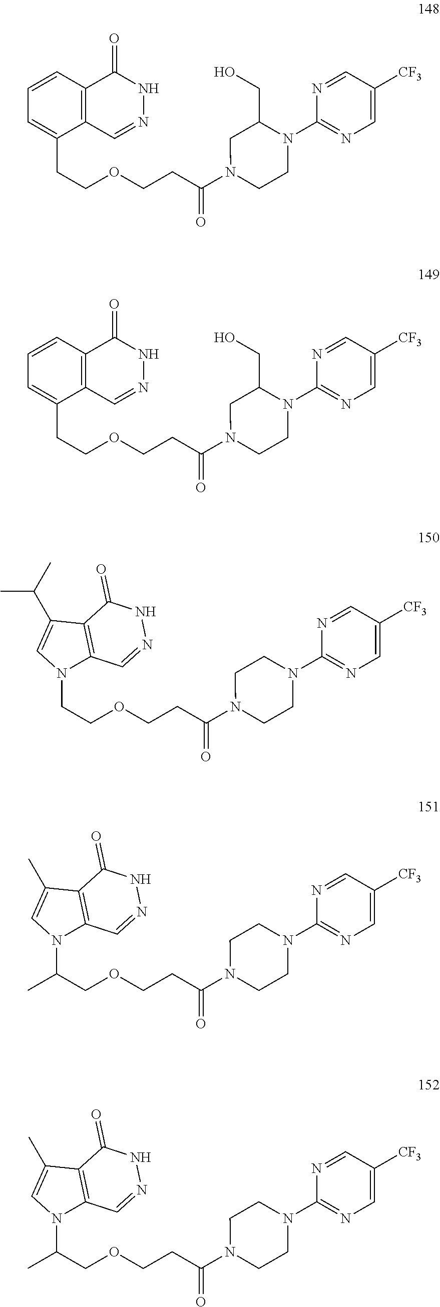 [SMILES: O=C(CCOCCc1cccc2c(=O)[nH]ncc12)N1CCN(c2ncc(C(F)(F)F)cn2)C(CO)C1 ; O=C(CCOCCc1cccc2c(=O)[nH]ncc12)N1CCN(c2ncc(C(F)(F)F)cn2)C(CO)C1 ; CC(C)c1cn(CCOCCC(=O)N2CCN(c3ncc(C(F)(F)F)cn3)CC2)c2cn[nH]c(=O)c12 ; Cc1cn(C(C)COCCC(=O)N2CCN(c3ncc(C(F)(F)F)cn3)CC2)c2cn[nH]c(=O)c12 ; Cc1cn(C(C)COCCC(=O)N2CCN(c3ncc(C(F)(F)F)cn3)CC2)c2cn[nH]c(=O)c12]

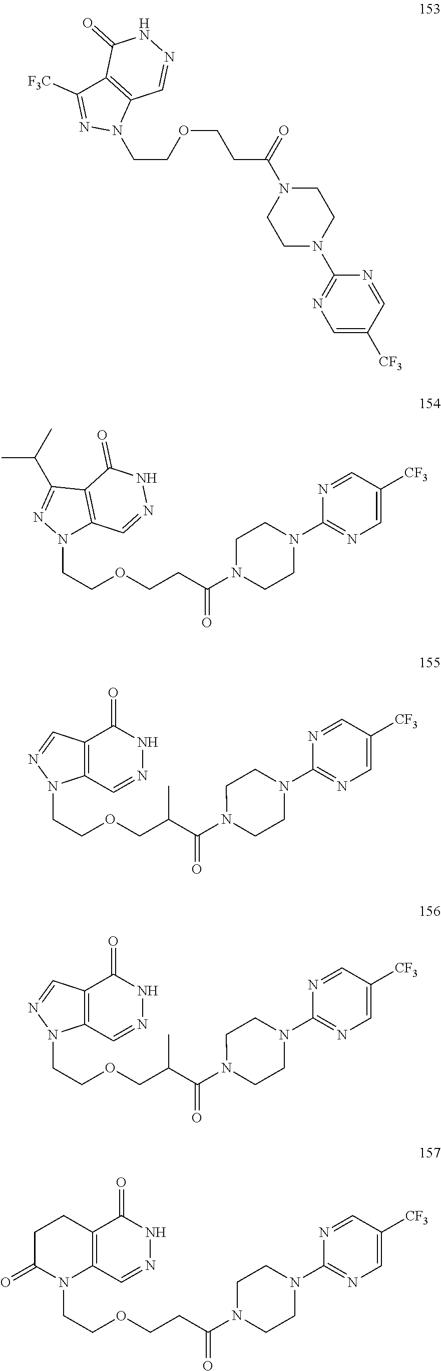 [SMILES: O=C(CCOCCn1nc(C(F)(F)F)c2c(=O)[nH]ncc21)N1CCN(c2ncc(C(F)(F)F)cn2)CC1 ; CC(C)c1nn(CCOCCC(=O)N2CCN(c3ncc(C(F)(F)F)cn3)CC2)c2cn[nH]c(=O)c12 ; CC(COCCn1ncc2c(=O)[nH]ncc21)C(=O)N1CCN(c2ncc(C(F)(F)F)cn2)CC1 ; CC(COCCn1ncc2c(=O)[nH]ncc21)C(=O)N1CCN(c2ncc(C(F)(F)F)cn2)CC1 ; O=C(CCOCCN1C(=O)CCc2c1cn[nH]c2=O)N1CCN(c2ncc(C(F)(F)F)cn2)CC1]

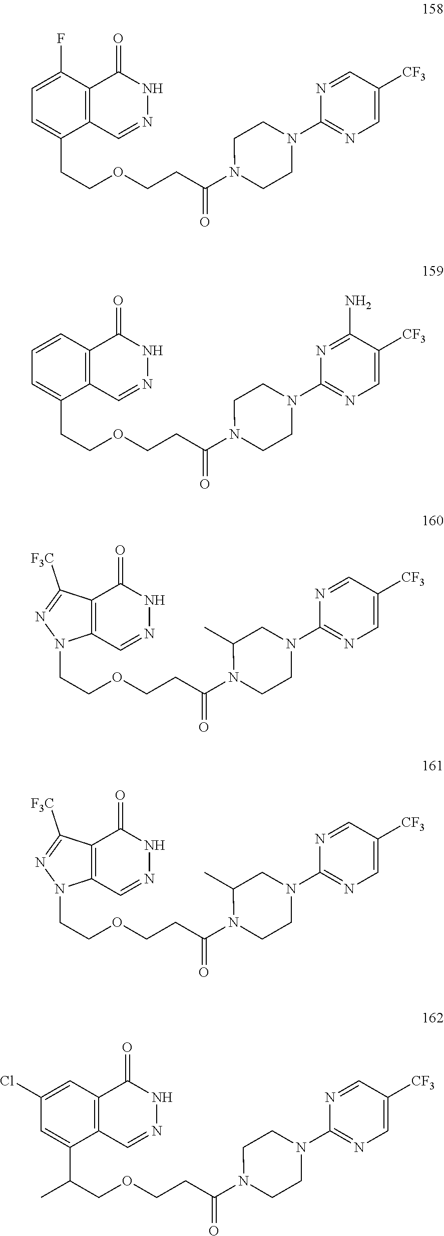 [SMILES: O=C(CCOCCc1ccc(F)c2c(=O)[nH]ncc12)N1CCN(c2ncc(C(F)(F)F)cn2)CC1 ; Nc1nc(N2CCN(C(=O)CCOCCc3cccc4c(=O)[nH]ncc34)CC2)ncc1C(F)(F)F ; CC1CN(c2ncc(C(F)(F)F)cn2)CCN1C(=O)CCOCCn1nc(C(F)(F)F)c2c(=O)[nH]ncc21 ; CC1CN(c2ncc(C(F)(F)F)cn2)CCN1C(=O)CCOCCn1nc(C(F)(F)F)c2c(=O)[nH]ncc21 ; CC(COCCC(=O)N1CCN(c2ncc(C(F)(F)F)cn2)CC1)c1cc(Cl)cc2c(=O)[nH]ncc12]

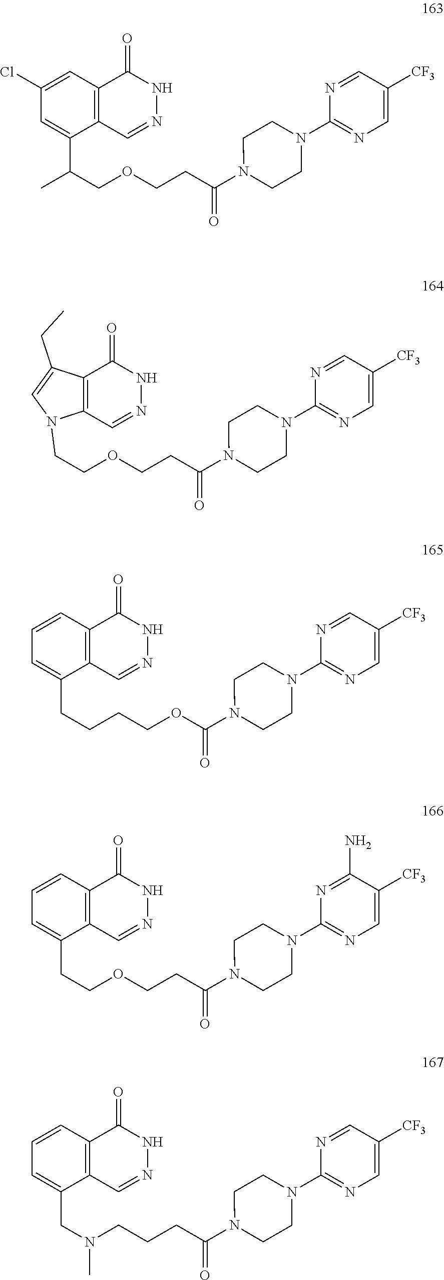 [SMILES: CC(COCCC(=O)N1CCN(c2ncc(C(F)(F)F)cn2)CC1)c1cc(Cl)cc2c(=O)[nH]ncc12 ; CCc1cn(CCOCCC(=O)N2CCN(c3ncc(C(F)(F)F)cn3)CC2)c2cn[nH]c(=O)c12 ; O=C(OCCCCc1cccc2c(=O)[nH]ncc12)N1CCN(c2ncc(C(F)(F)F)cn2)CC1 ; Nc1nc(N2CCN(C(=O)CCOCCc3cccc4c(=O)[nH]ncc34)CC2)ncc1C(F)(F)F ; CN(CCCC(=O)N1CCN(c2ncc(C(F)(F)F)cn2)CC1)Cc1cccc2c(=O)[nH]ncc12]

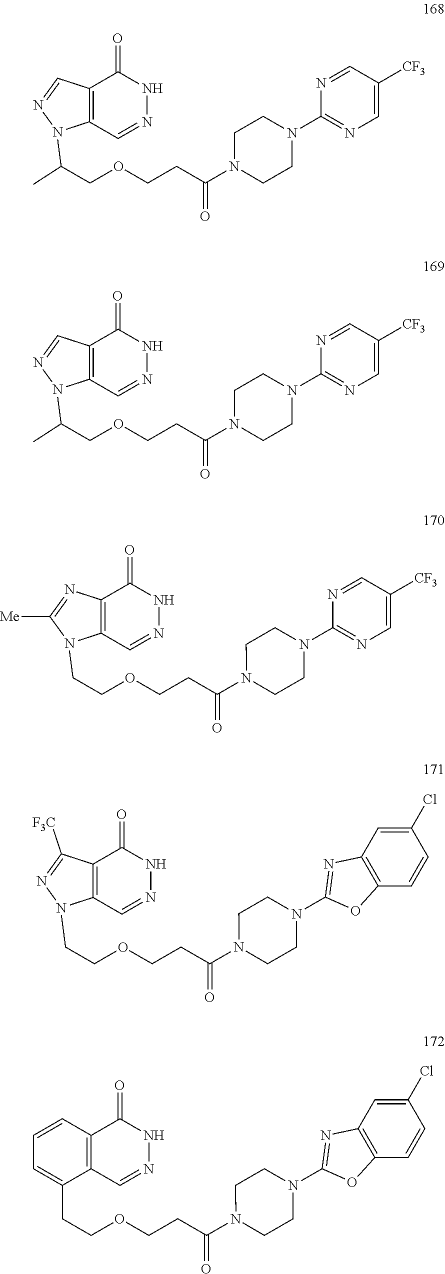 [SMILES: CC(COCCC(=O)N1CCN(c2ncc(C(F)(F)F)cn2)CC1)n1ncc2c(=O)[nH]ncc21 ; CC(COCCC(=O)N1CCN(c2ncc(C(F)(F)F)cn2)CC1)n1ncc2c(=O)[nH]ncc21 ; Cc1nc2c(=O)[nH]ncc2n1CCOCCC(=O)N1CCN(c2ncc(C(F)(F)F)cn2)CC1 ; O=C(CCOCCn1nc(C(F)(F)F)c2c(=O)[nH]ncc21)N1CCN(c2nc3cc(Cl)ccc3o2)CC1 ; O=C(CCOCCc1cccc2c(=O)[nH]ncc12)N1CCN(c2nc3cc(Cl)ccc3o2)CC1]

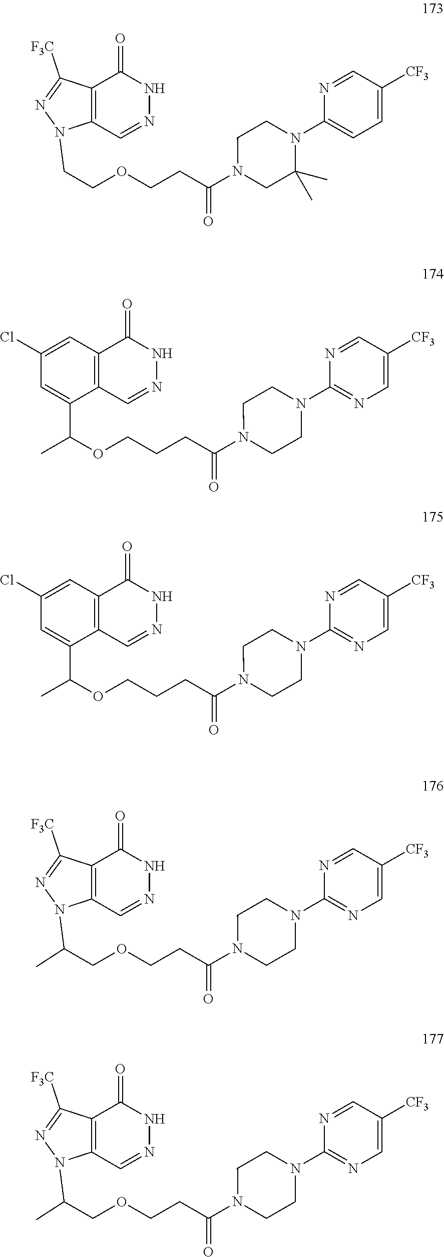 [SMILES: CC1(C)CN(C(=O)CCOCCn2nc(C(F)(F)F)c3c(=O)[nH]ncc32)CCN1c1ccc(C(F)(F)F)cn1 ; CC(OCCCC(=O)N1CCN(c2ncc(C(F)(F)F)cn2)CC1)c1cc(Cl)cc2c(=O)[nH]ncc12 ; CC(OCCCC(=O)N1CCN(c2ncc(C(F)(F)F)cn2)CC1)c1cc(Cl)cc2c(=O)[nH]ncc12 ; CC(COCCC(=O)N1CCN(c2ncc(C(F)(F)F)cn2)CC1)n1nc(C(F)(F)F)c2c(=O)[nH]ncc21 ; CC(COCCC(=O)N1CCN(c2ncc(C(F)(F)F)cn2)CC1)n1nc(C(F)(F)F)c2c(=O)[nH]ncc21]

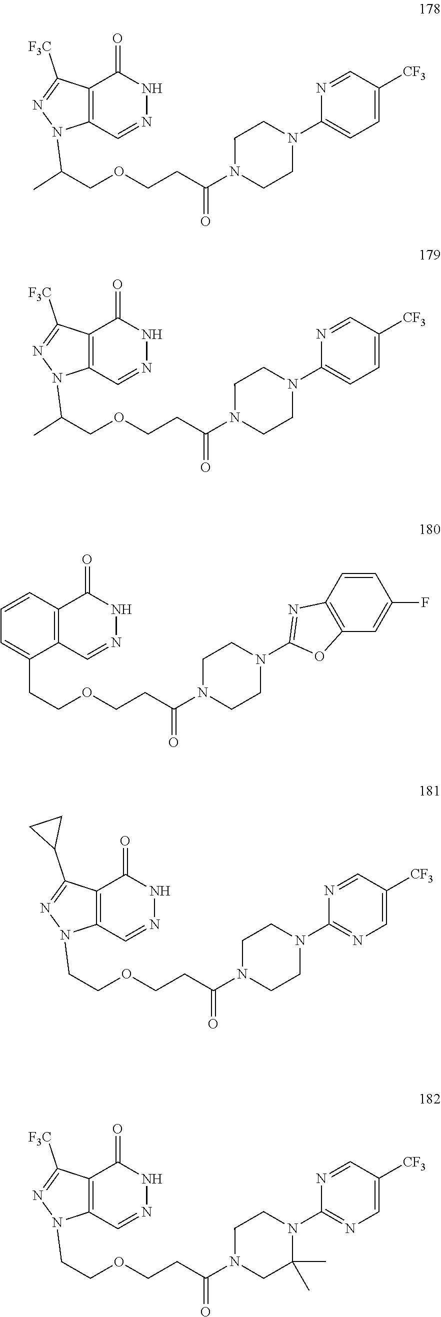 [SMILES: CC(COCCC(=O)N1CCN(c2ccc(C(F)(F)F)cn2)CC1)n1nc(C(F)(F)F)c2c(=O)[nH]ncc21 ; CC(COCCC(=O)N1CCN(c2ccc(C(F)(F)F)cn2)CC1)n1nc(C(F)(F)F)c2c(=O)[nH]ncc21 ; O=C(CCOCCc1cccc2c(=O)[nH]ncc12)N1CCN(c2nc3ccc(F)cc3o2)CC1 ; O=C(CCOCCn1nc(C2CC2)c2c(=O)[nH]ncc21)N1CCN(c2ncc(C(F)(F)F)cn2)CC1 ; CC1(C)CN(C(=O)CCOCCn2nc(C(F)(F)F)c3c(=O)[nH]ncc32)CCN1c1ncc(C(F)(F)F)cn1]

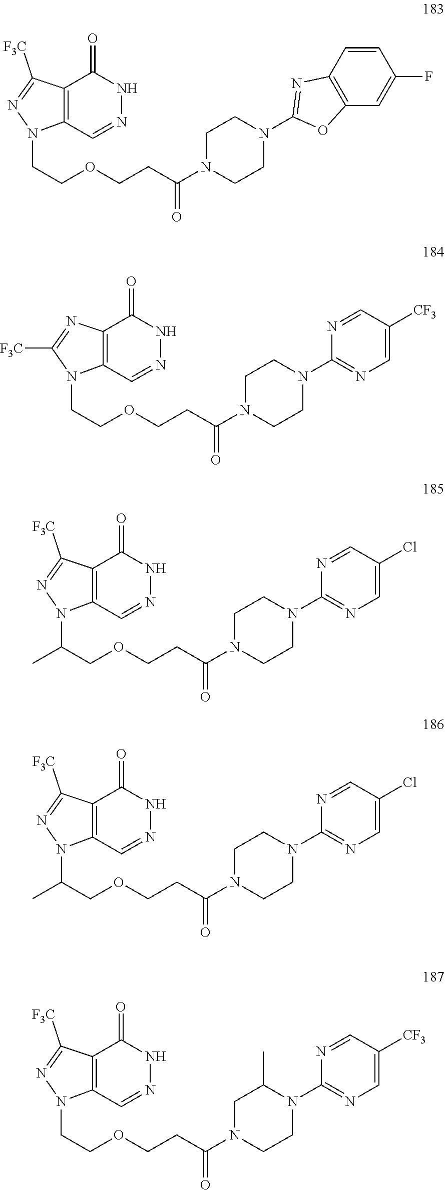

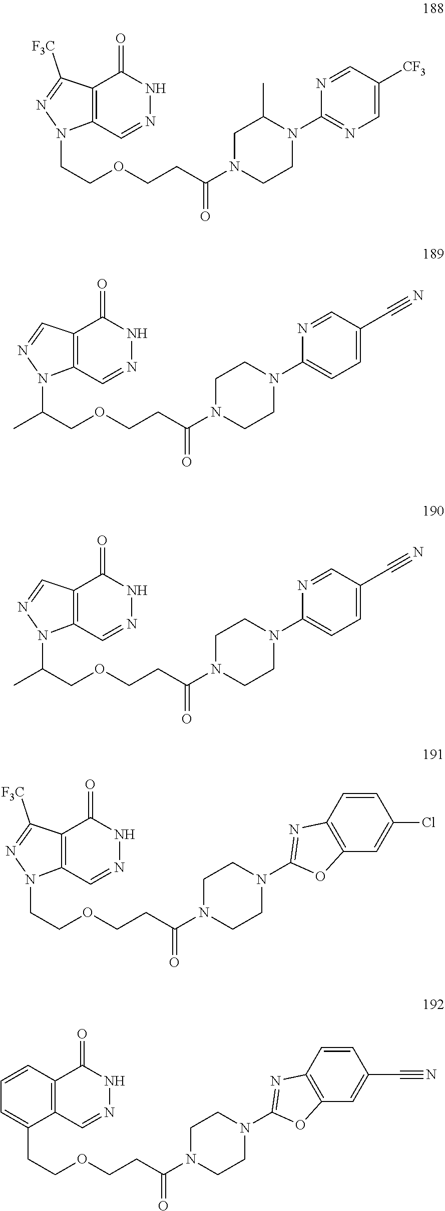

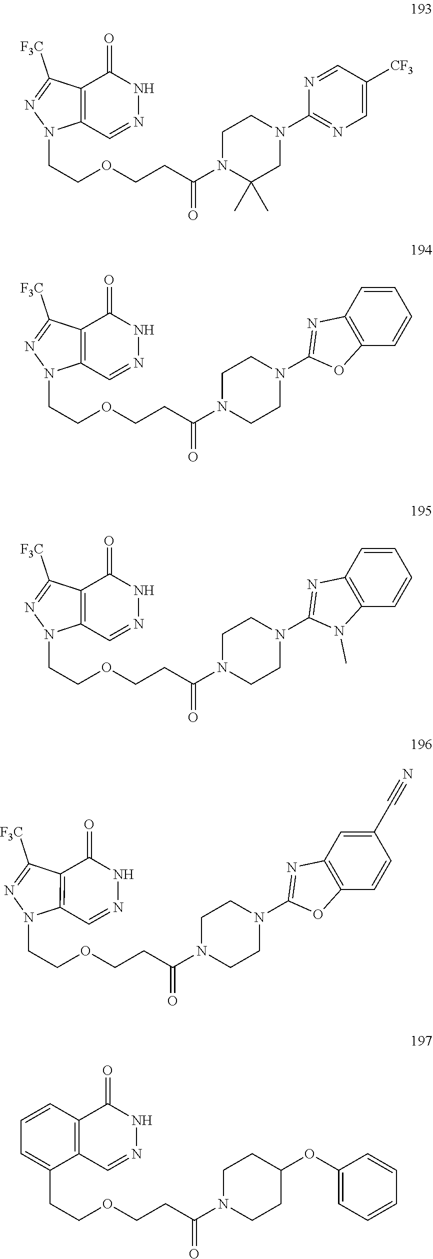

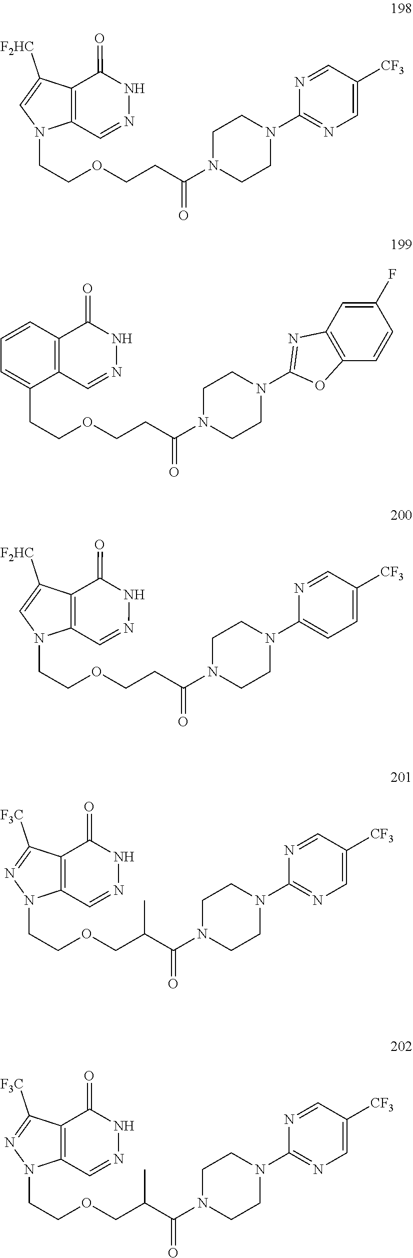

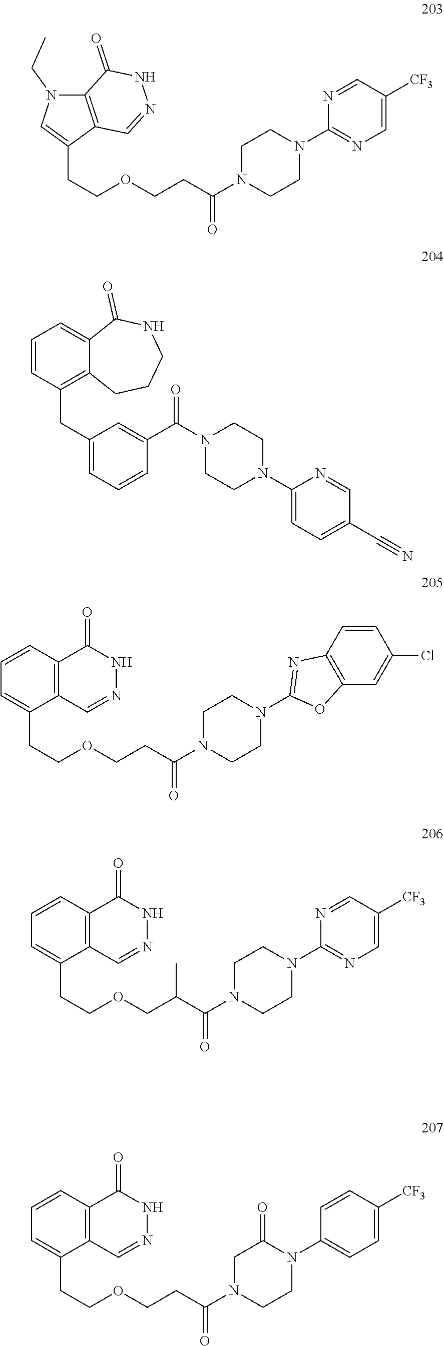 [SMILES: CCn1cc(CCOCCC(=O)N2CCN(c3ncc(C(F)(F)F)cn3)CC2)c2cn[nH]c(=O)c21 ; N#Cc1ccc(N2CCN(C(=O)c3cccc(Cc4cccc5c4CCCNC5=O)c3)CC2)nc1 ; O=C(CCOCCc1cccc2c(=O)[nH]ncc12)N1CCN(c2nc3ccc(Cl)cc3o2)CC1 ; CC(COCCc1cccc2c(=O)[nH]ncc12)C(=O)N1CCN(c2ncc(C(F)(F)F)cn2)CC1 ; O=C(CCOCCc1cccc2c(=O)[nH]ncc12)N1CCN(c2ccc(C(F)(F)F)cc2)C(=O)C1]

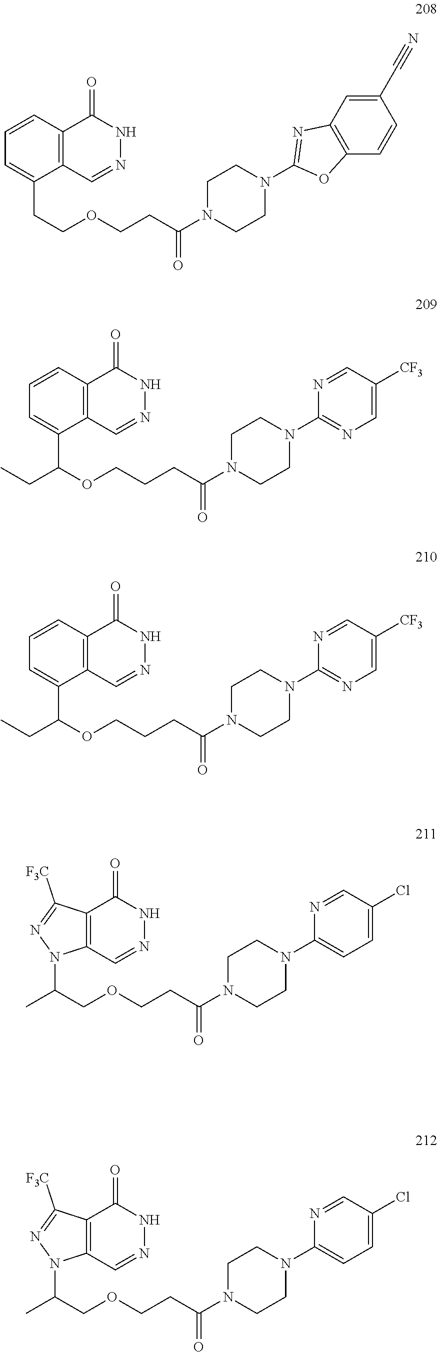

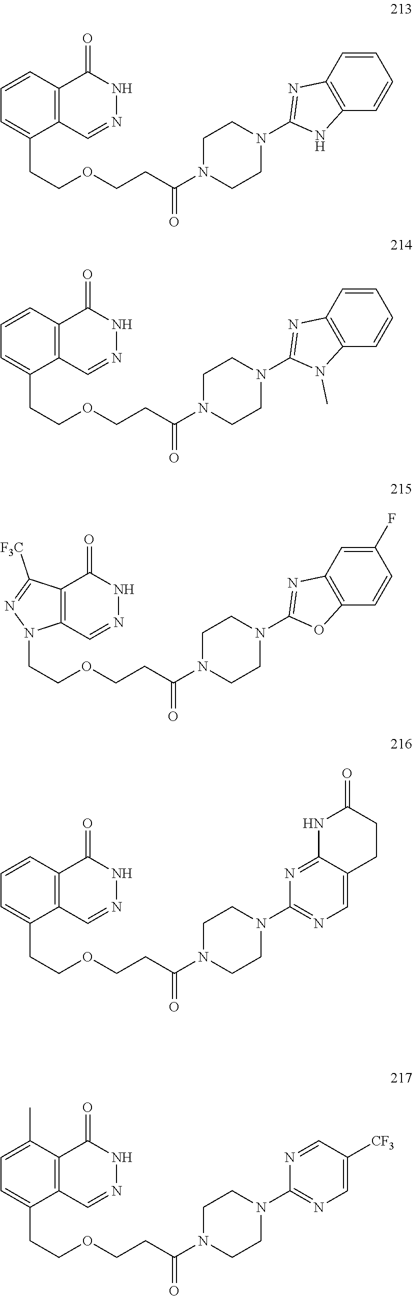

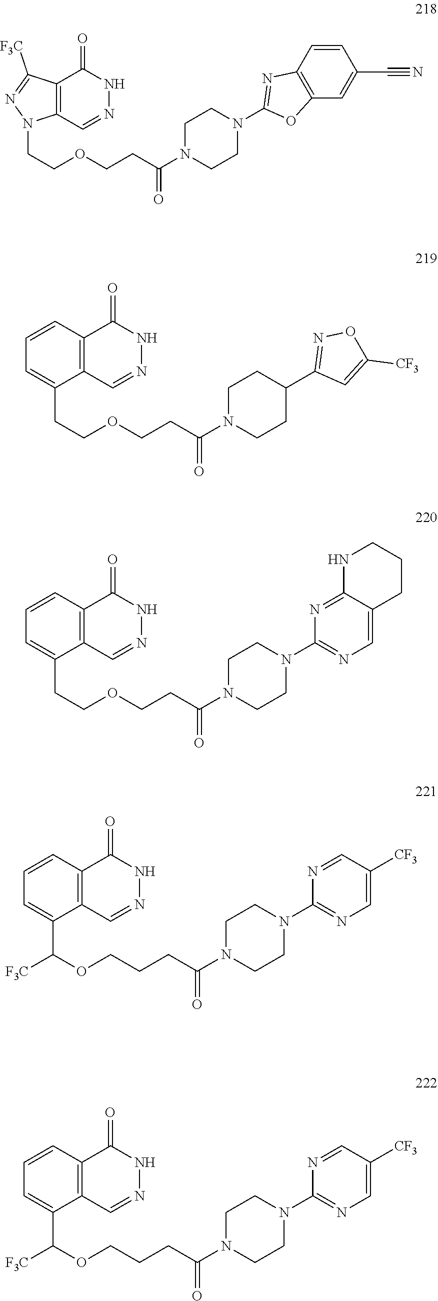

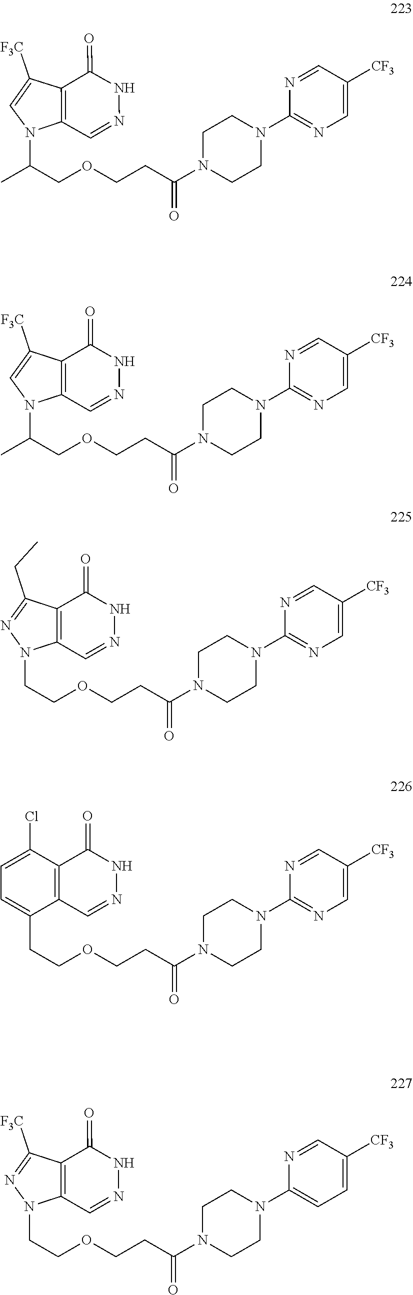 [SMILES: CC(COCCC(=O)N1CCN(c2ncc(C(F)(F)F)cn2)CC1)n1cc(C(F)(F)F)c2c(=O)[nH]ncc21 ; CC(COCCC(=O)N1CCN(c2ncc(C(F)(F)F)cn2)CC1)n1cc(C(F)(F)F)c2c(=O)[nH]ncc21 ; CCc1nn(CCOCCC(=O)N2CCN(c3ncc(C(F)(F)F)cn3)CC2)c2cn[nH]c(=O)c12 ; O=C(CCOCCc1ccc(Cl)c2c(=O)[nH]ncc12)N1CCN(c2ncc(C(F)(F)F)cn2)CC1 ; O=C(CCOCCn1nc(C(F)(F)F)c2c(=O)[nH]ncc21)N1CCN(c2ccc(C(F)(F)F)cn2)CC1]

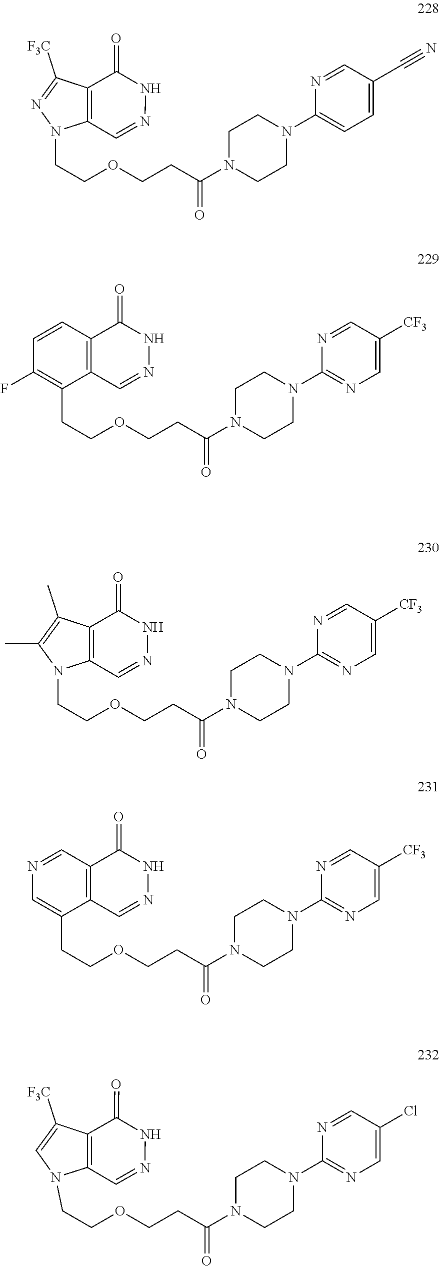

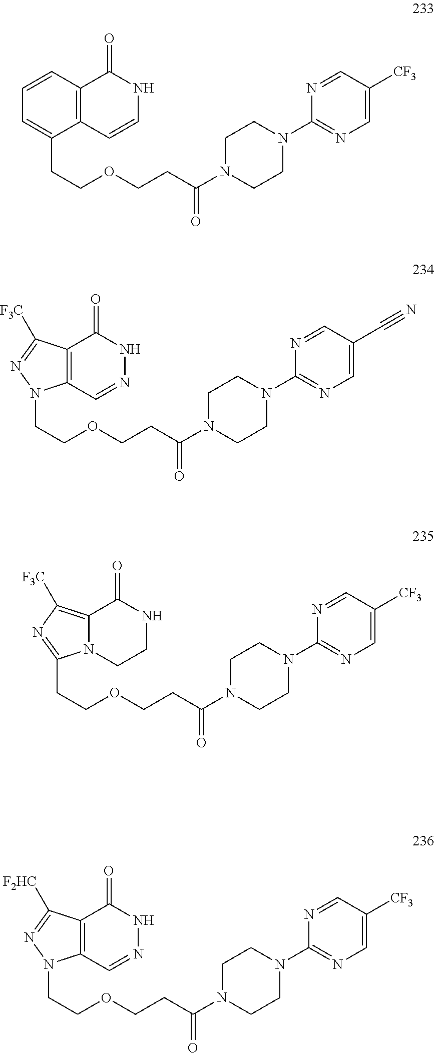 [SMILES: O=C(CCOCCc1cccc2c(=O)[nH]ccc12)N1CCN(c2ncc(C(F)(F)F)cn2)CC1 ; N#Cc1cnc(N2CCN(C(=O)CCOCCn3nc(C(F)(F)F)c4c(=O)[nH]ncc43)CC2)nc1 ; O=C1NCCn2c(CCOCCC(=O)N3CCN(c4ncc(C(F)(F)F)cn4)CC3)nc(C(F)(F)F)c21 ; O=C(CCOCCn1nc(C(F)F)c2c(=O)[nH]ncc21)N1CCN(c2ncc(C(F)(F)F)cn2)CC1]

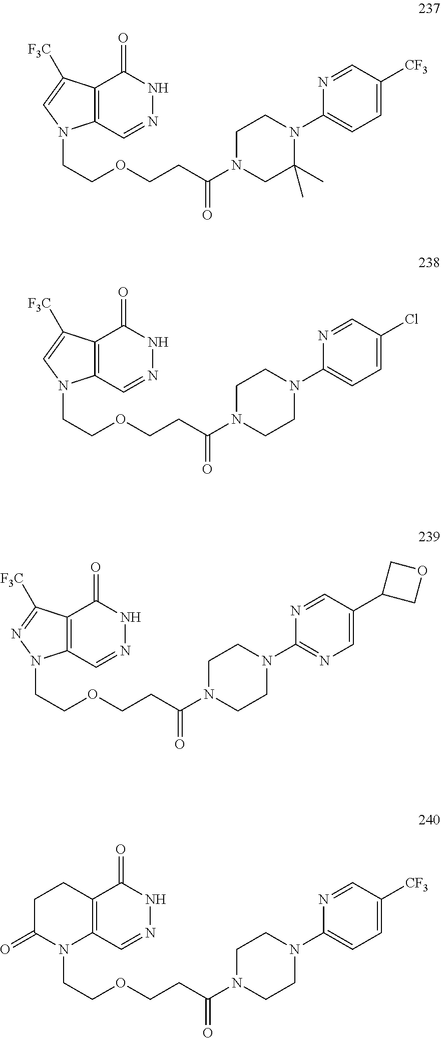 [SMILES: CC1(C)CN(C(=O)CCOCCn2cc(C(F)(F)F)c3c(=O)[nH]ncc32)CCN1c1ccc(C(F)(F)F)cn1 ; O=C(CCOCCn1cc(C(F)(F)F)c2c(=O)[nH]ncc21)N1CCN(c2ccc(Cl)cn2)CC1 ; O=C(CCOCCn1nc(C(F)(F)F)c2c(=O)[nH]ncc21)N1CCN(c2ncc(C3COC3)cn2)CC1 ; O=C(CCOCCN1C(=O)CCc2c1cn[nH]c2=O)N1CCN(c2ccc(C(F)(F)F)cn2)CC1]

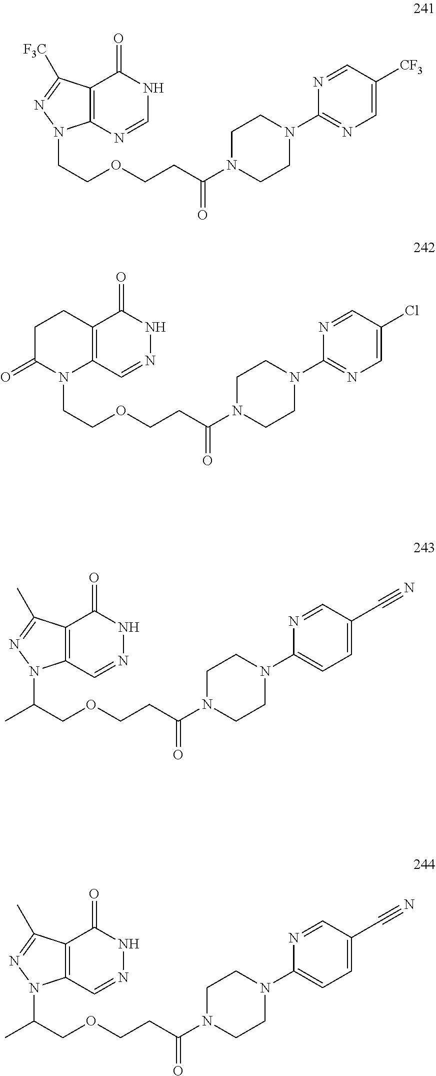 [SMILES: O=C(CCOCCn1nc(C(F)(F)F)c2c(=O)[nH]cnc21)N1CCN(c2ncc(C(F)(F)F)cn2)CC1 ; O=C(CCOCCN1C(=O)CCc2c1cn[nH]c2=O)N1CCN(c2ncc(Cl)cn2)CC1 ; Cc1nn(C(C)COCCC(=O)N2CCN(c3ccc(C#N)cn3)CC2)c2cn[nH]c(=O)c12 ; Cc1nn(C(C)COCCC(=O)N2CCN(c3ccc(C#N)cn3)CC2)c2cn[nH]c(=O)c12]

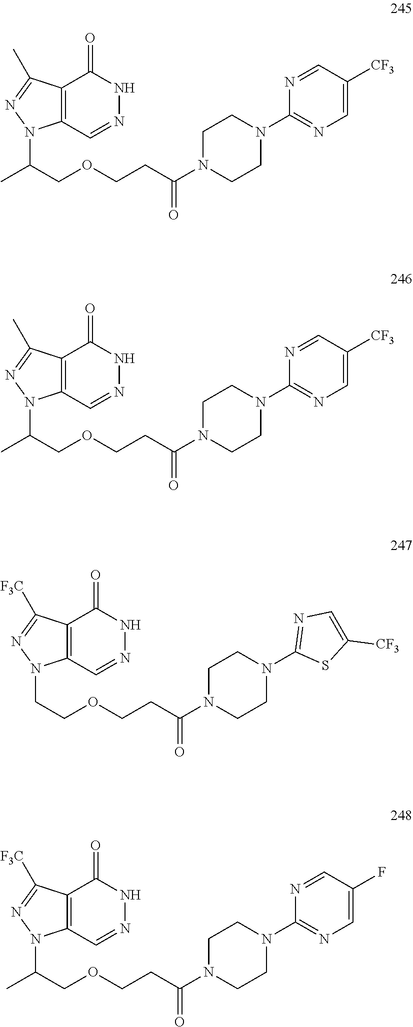 [SMILES: Cc1nn(C(C)COCCC(=O)N2CCN(c3ncc(C(F)(F)F)cn3)CC2)c2cn[nH]c(=O)c12 ; Cc1nn(C(C)COCCC(=O)N2CCN(c3ncc(C(F)(F)F)cn3)CC2)c2cn[nH]c(=O)c12 ; O=C(CCOCCn1nc(C(F)(F)F)c2c(=O)[nH]ncc21)N1CCN(c2ncc(C(F)(F)F)s2)CC1 ; CC(COCCC(=O)N1CCN(c2ncc(F)cn2)CC1)n1nc(C(F)(F)F)c2c(=O)[nH]ncc21]

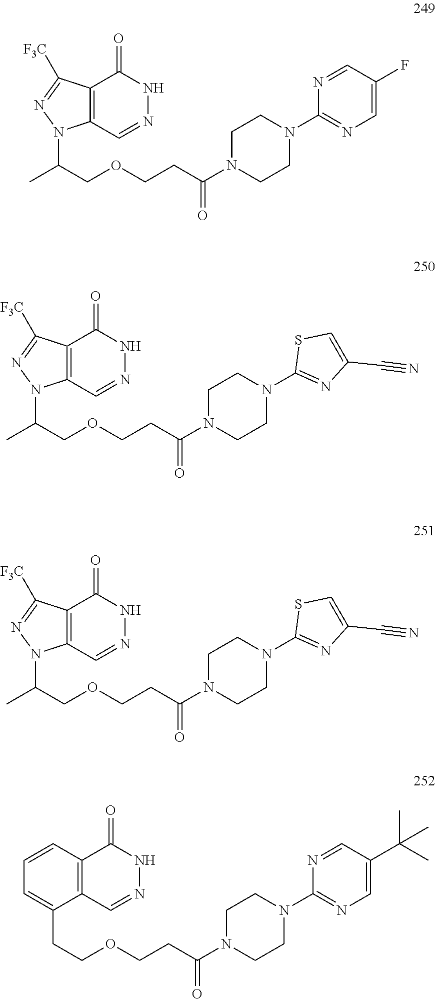 [SMILES: CC(COCCC(=O)N1CCN(c2ncc(F)cn2)CC1)n1nc(C(F)(F)F)c2c(=O)[nH]ncc21 ; CC(COCCC(=O)N1CCN(c2nc(C#N)cs2)CC1)n1nc(C(F)(F)F)c2c(=O)[nH]ncc21 ; CC(COCCC(=O)N1CCN(c2nc(C#N)cs2)CC1)n1nc(C(F)(F)F)c2c(=O)[nH]ncc21 ; CC(C)(C)c1cnc(N2CCN(C(=O)CCOCCc3cccc4c(=O)[nH]ncc34)CC2)nc1]

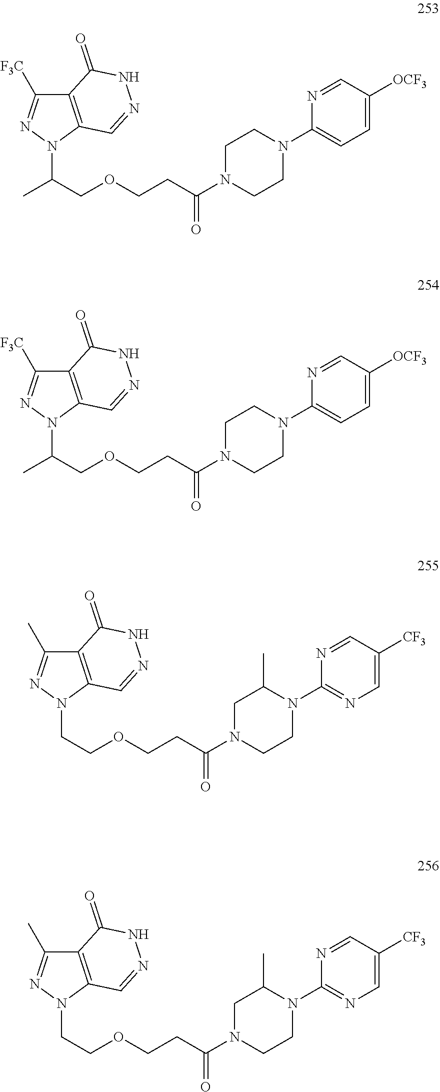 [SMILES: CC(COCCC(=O)N1CCN(c2ccc(OC(F)(F)F)cn2)CC1)n1nc(C(F)(F)F)c2c(=O)[nH]ncc21 ; CC(COCCC(=O)N1CCN(c2ccc(OC(F)(F)F)cn2)CC1)n1nc(C(F)(F)F)c2c(=O)[nH]ncc21 ; Cc1nn(CCOCCC(=O)N2CCN(c3ncc(C(F)(F)F)cn3)C(C)C2)c2cn[nH]c(=O)c12 ; Cc1nn(CCOCCC(=O)N2CCN(c3ncc(C(F)(F)F)cn3)C(C)C2)c2cn[nH]c(=O)c12]

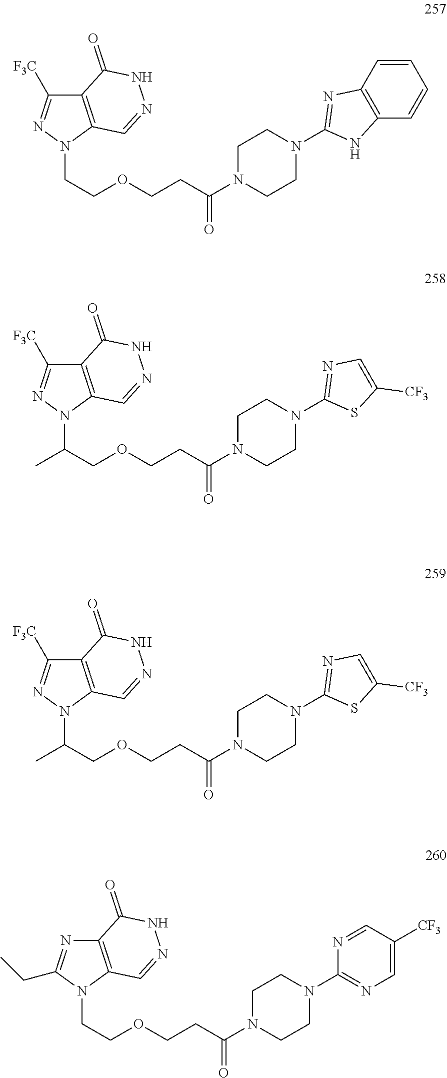 [SMILES: O=C(CCOCCn1nc(C(F)(F)F)c2c(=O)[nH]ncc21)N1CCN(c2nc3ccccc3[nH]2)CC1 ; CC(COCCC(=O)N1CCN(c2ncc(C(F)(F)F)s2)CC1)n1nc(C(F)(F)F)c2c(=O)[nH]ncc21 ; CC(COCCC(=O)N1CCN(c2ncc(C(F)(F)F)s2)CC1)n1nc(C(F)(F)F)c2c(=O)[nH]ncc21 ; CCc1nc2c(=O)[nH]ncc2n1CCOCCC(=O)N1CCN(c2ncc(C(F)(F)F)cn2)CC1]

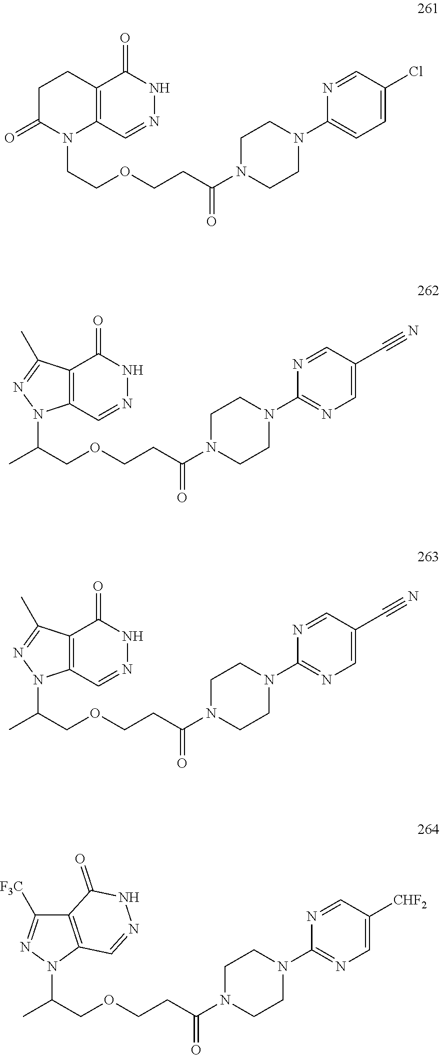 [SMILES: O=C(CCOCCN1C(=O)CCc2c1cn[nH]c2=O)N1CCN(c2ccc(Cl)cn2)CC1 ; Cc1nn(C(C)COCCC(=O)N2CCN(c3ncc(C#N)cn3)CC2)c2cn[nH]c(=O)c12 ; Cc1nn(C(C)COCCC(=O)N2CCN(c3ncc(C#N)cn3)CC2)c2cn[nH]c(=O)c12 ; CC(COCCC(=O)N1CCN(c2ncc(C(F)F)cn2)CC1)n1nc(C(F)(F)F)c2c(=O)[nH]ncc21]

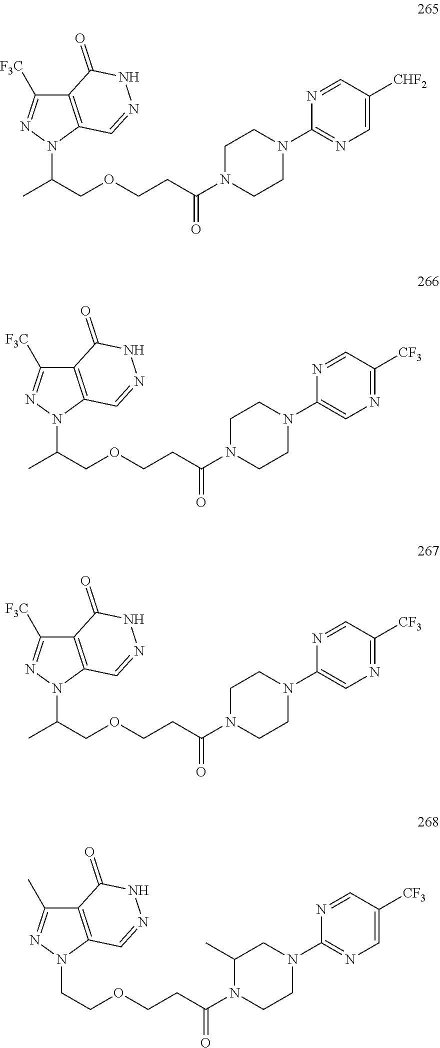 [SMILES: CC(COCCC(=O)N1CCN(c2ncc(C(F)F)cn2)CC1)n1nc(C(F)(F)F)c2c(=O)[nH]ncc21 ; CC(COCCC(=O)N1CCN(c2cnc(C(F)(F)F)cn2)CC1)n1nc(C(F)(F)F)c2c(=O)[nH]ncc21 ; CC(COCCC(=O)N1CCN(c2cnc(C(F)(F)F)cn2)CC1)n1nc(C(F)(F)F)c2c(=O)[nH]ncc21 ; Cc1nn(CCOCCC(=O)N2CCN(c3ncc(C(F)(F)F)cn3)CC2C)c2cn[nH]c(=O)c12]

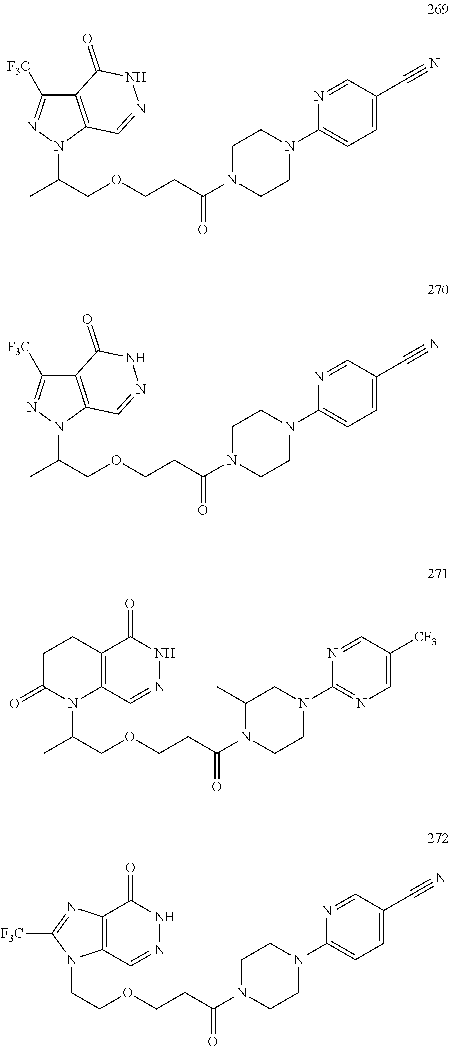 [SMILES: CC(COCCC(=O)N1CCN(c2ccc(C#N)cn2)CC1)n1nc(C(F)(F)F)c2c(=O)[nH]ncc21 ; CC(COCCC(=O)N1CCN(c2ccc(C#N)cn2)CC1)n1nc(C(F)(F)F)c2c(=O)[nH]ncc21 ; CC1CN(c2ncc(C(F)(F)F)cn2)CCN1C(=O)CCOCC(C)N1C(=O)CCc2c1cn[nH]c2=O ; N#Cc1ccc(N2CCN(C(=O)CCOCCn3c(C(F)(F)F)nc4c(=O)[nH]ncc43)CC2)nc1]

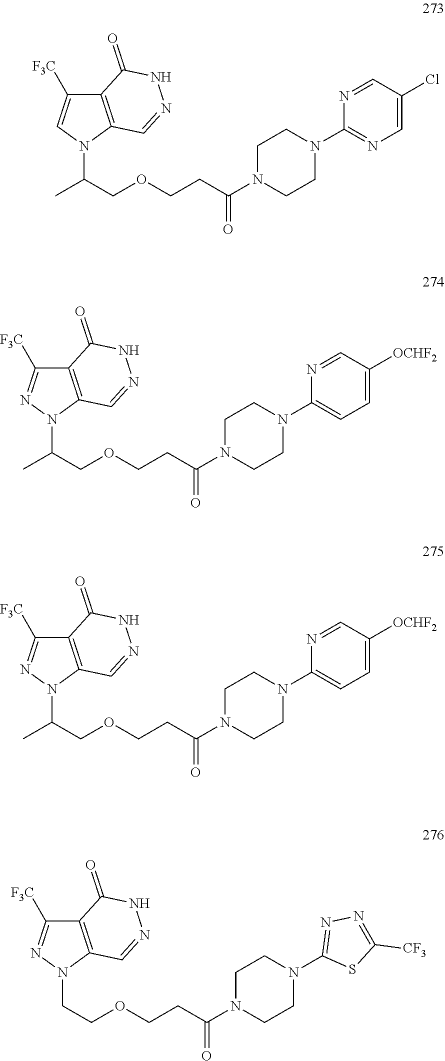 [SMILES: CC(COCCC(=O)N1CCN(c2ncc(Cl)cn2)CC1)n1cc(C(F)(F)F)c2c(=O)[nH]ncc21 ; CC(COCCC(=O)N1CCN(c2ccc(OC(F)F)cn2)CC1)n1nc(C(F)(F)F)c2c(=O)[nH]ncc21 ; CC(COCCC(=O)N1CCN(c2ccc(OC(F)F)cn2)CC1)n1nc(C(F)(F)F)c2c(=O)[nH]ncc21 ; O=C(CCOCCn1nc(C(F)(F)F)c2c(=O)[nH]ncc21)N1CCN(c2nnc(C(F)(F)F)s2)CC1]

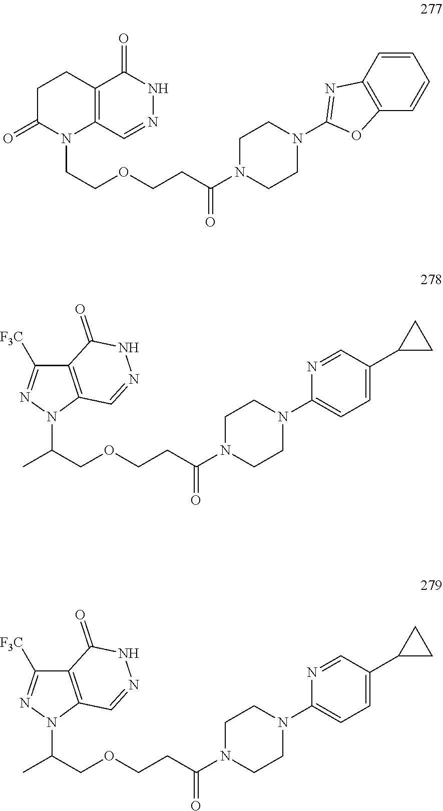 [SMILES: O=C(CCOCCN1C(=O)CCc2c1cn[nH]c2=O)N1CCN(c2nc3ccccc3o2)CC1 ; CC(COCCC(=O)N1CCN(c2ccc(C3CC3)cn2)CC1)n1nc(C(F)(F)F)c2c(=O)[nH]ncc21 ; CC(COCCC(=O)N1CCN(c2ccc(C3CC3)cn2)CC1)n1nc(C(F)(F)F)c2c(=O)[nH]ncc21]

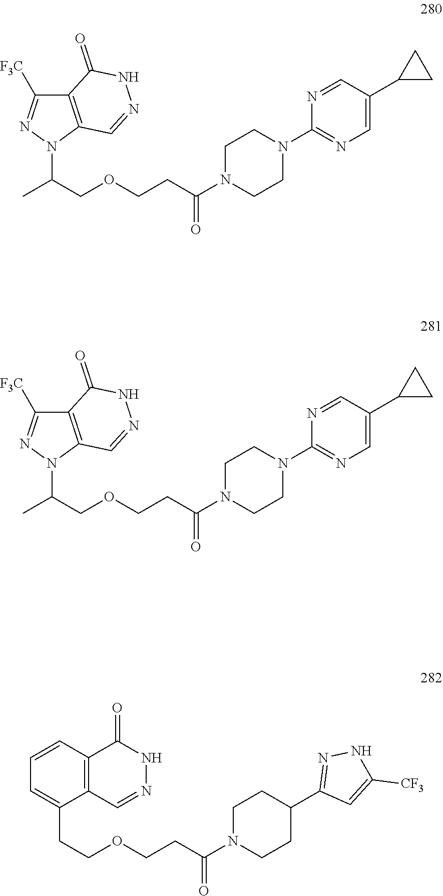 [SMILES: CC(COCCC(=O)N1CCN(c2ncc(C3CC3)cn2)CC1)n1nc(C(F)(F)F)c2c(=O)[nH]ncc21 ; CC(COCCC(=O)N1CCN(c2ncc(C3CC3)cn2)CC1)n1nc(C(F)(F)F)c2c(=O)[nH]ncc21 ; O=C(CCOCCc1cccc2c(=O)[nH]ncc12)N1CCC(c2cc(C(F)(F)F)[nH]n2)CC1]

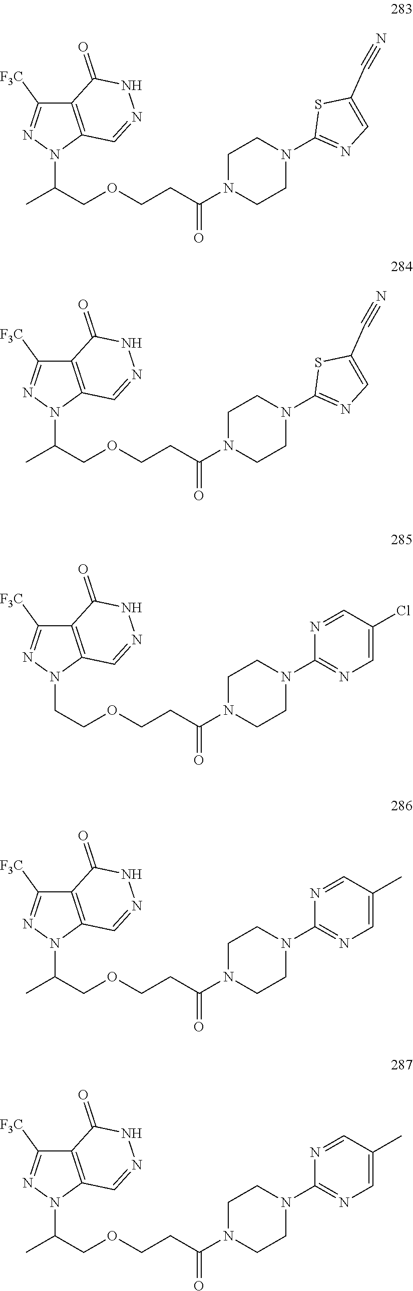 [SMILES: CC(COCCC(=O)N1CCN(c2ncc(C#N)s2)CC1)n1nc(C(F)(F)F)c2c(=O)[nH]ncc21 ; CC(COCCC(=O)N1CCN(c2ncc(C#N)s2)CC1)n1nc(C(F)(F)F)c2c(=O)[nH]ncc21 ; O=C(CCOCCn1nc(C(F)(F)F)c2c(=O)[nH]ncc21)N1CCN(c2ncc(Cl)cn2)CC1 ; Cc1cnc(N2CCN(C(=O)CCOCC(C)n3nc(C(F)(F)F)c4c(=O)[nH]ncc43)CC2)nc1 ; Cc1cnc(N2CCN(C(=O)CCOCC(C)n3nc(C(F)(F)F)c4c(=O)[nH]ncc43)CC2)nc1]

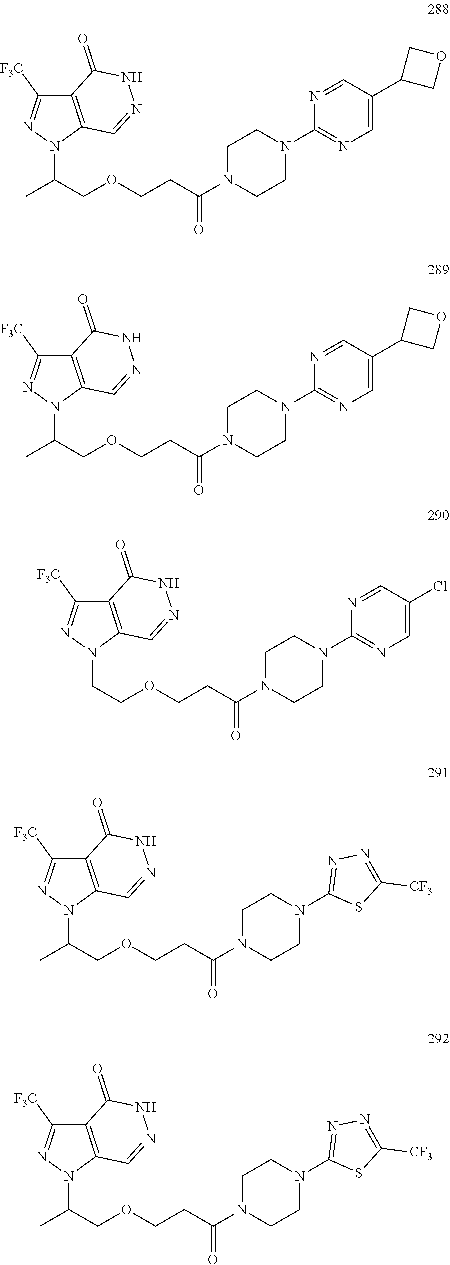 [SMILES: CC(COCCC(=O)N1CCN(c2ncc(C3COC3)cn2)CC1)n1nc(C(F)(F)F)c2c(=O)[nH]ncc21 ; CC(COCCC(=O)N1CCN(c2ncc(C3COC3)cn2)CC1)n1nc(C(F)(F)F)c2c(=O)[nH]ncc21 ; O=C(CCOCCn1nc(C(F)(F)F)c2c(=O)[nH]ncc21)N1CCN(c2ncc(Cl)cn2)CC1 ; CC(COCCC(=O)N1CCN(c2nnc(C(F)(F)F)s2)CC1)n1nc(C(F)(F)F)c2c(=O)[nH]ncc21 ; CC(COCCC(=O)N1CCN(c2nnc(C(F)(F)F)s2)CC1)n1nc(C(F)(F)F)c2c(=O)[nH]ncc21]

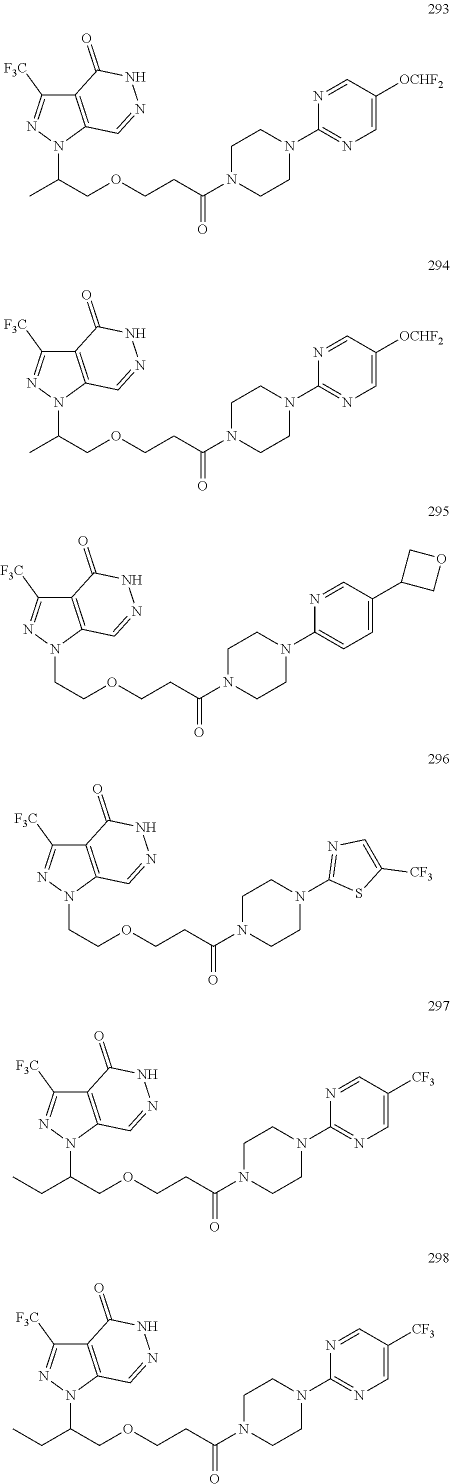 [SMILES: CC(COCCC(=O)N1CCN(c2ncc(OC(F)F)cn2)CC1)n1nc(C(F)(F)F)c2c(=O)[nH]ncc21 ; CC(COCCC(=O)N1CCN(c2ncc(OC(F)F)cn2)CC1)n1nc(C(F)(F)F)c2c(=O)[nH]ncc21 ; O=C(CCOCCn1nc(C(F)(F)F)c2c(=O)[nH]ncc21)N1CCN(c2ccc(C3COC3)cn2)CC1 ; O=C(CCOCCn1nc(C(F)(F)F)c2c(=O)[nH]ncc21)N1CCN(c2ncc(C(F)(F)F)s2)CC1 ; CCC(COCCC(=O)N1CCN(c2ncc(C(F)(F)F)cn2)CC1)n1nc(C(F)(F)F)c2c(=O)[nH]ncc21 ; CCC(COCCC(=O)N1CCN(c2ncc(C(F)(F)F)cn2)CC1)n1nc(C(F)(F)F)c2c(=O)[nH]ncc21]

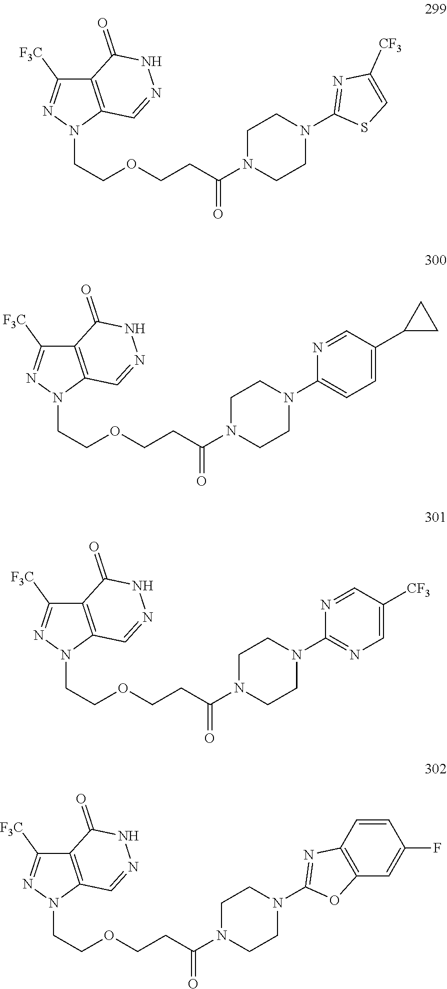 [SMILES: O=C(CCOCCn1nc(C(F)(F)F)c2c(=O)[nH]ncc21)N1CCN(c2nc(C(F)(F)F)cs2)CC1 ; O=C(CCOCCn1nc(C(F)(F)F)c2c(=O)[nH]ncc21)N1CCN(c2ccc(C3CC3)cn2)CC1 ; O=C(CCOCCn1nc(C(F)(F)F)c2c(=O)[nH]ncc21)N1CCN(c2ncc(C(F)(F)F)cn2)CC1 ; O=C(CCOCCn1nc(C(F)(F)F)c2c(=O)[nH]ncc21)N1CCN(c2nc3ccc(F)cc3o2)CC1]

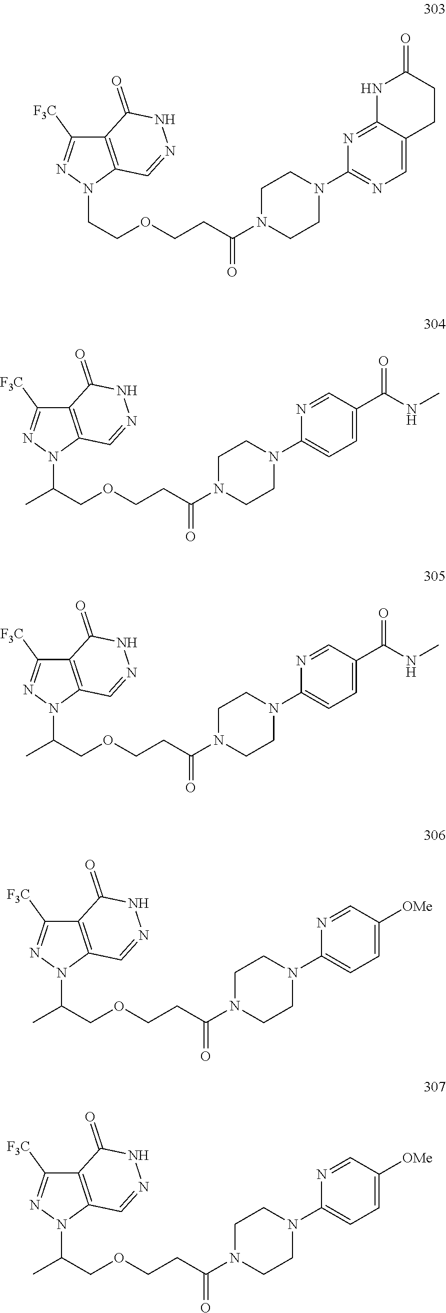 [SMILES: O=C1CCc2cnc(N3CCN(C(=O)CCOCCn4nc(C(F)(F)F)c5c(=O)[nH]ncc54)CC3)nc2N1 ; CNC(=O)c1ccc(N2CCN(C(=O)CCOCC(C)n3nc(C(F)(F)F)c4c(=O)[nH]ncc43)CC2)nc1 ; CNC(=O)c1ccc(N2CCN(C(=O)CCOCC(C)n3nc(C(F)(F)F)c4c(=O)[nH]ncc43)CC2)nc1 ; COc1ccc(N2CCN(C(=O)CCOCC(C)n3nc(C(F)(F)F)c4c(=O)[nH]ncc43)CC2)nc1 ; COc1ccc(N2CCN(C(=O)CCOCC(C)n3nc(C(F)(F)F)c4c(=O)[nH]ncc43)CC2)nc1]

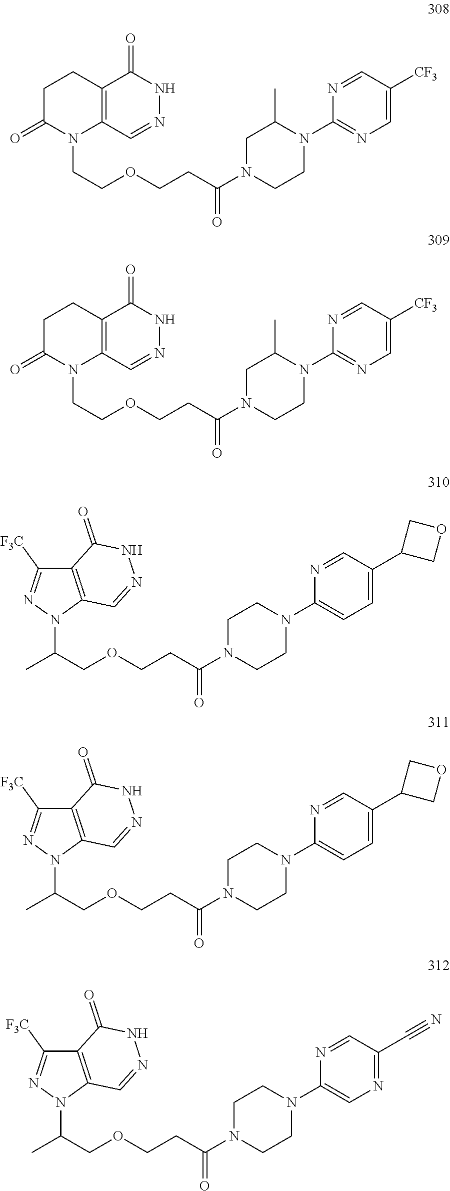 [SMILES: CC1CN(C(=O)CCOCCN2C(=O)CCc3c2cn[nH]c3=O)CCN1c1ncc(C(F)(F)F)cn1 ; CC1CN(C(=O)CCOCCN2C(=O)CCc3c2cn[nH]c3=O)CCN1c1ncc(C(F)(F)F)cn1 ; CC(COCCC(=O)N1CCN(c2ccc(C3COC3)cn2)CC1)n1nc(C(F)(F)F)c2c(=O)[nH]ncc21 ; CC(COCCC(=O)N1CCN(c2ccc(C3COC3)cn2)CC1)n1nc(C(F)(F)F)c2c(=O)[nH]ncc21 ; CC(COCCC(=O)N1CCN(c2cnc(C#N)cn2)CC1)n1nc(C(F)(F)F)c2c(=O)[nH]ncc21]

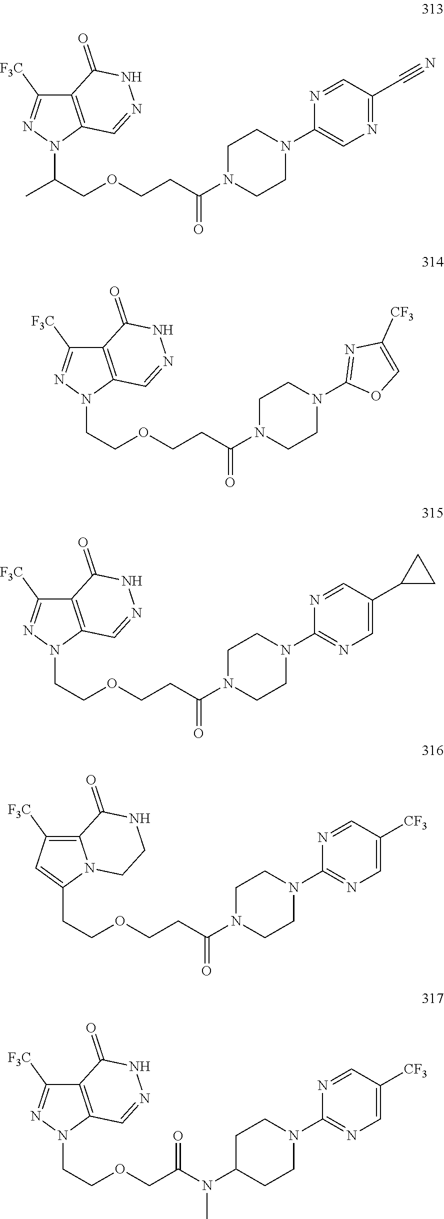 [SMILES: CC(COCCC(=O)N1CCN(c2cnc(C#N)cn2)CC1)n1nc(C(F)(F)F)c2c(=O)[nH]ncc21 ; O=C(CCOCCn1nc(C(F)(F)F)c2c(=O)[nH]ncc21)N1CCN(c2nc(C(F)(F)F)co2)CC1 ; O=C(CCOCCn1nc(C(F)(F)F)c2c(=O)[nH]ncc21)N1CCN(c2ncc(C3CC3)cn2)CC1 ; O=C1NCCn2c(CCOCCC(=O)N3CCN(c4ncc(C(F)(F)F)cn4)CC3)cc(C(F)(F)F)c21 ; CN(C(=O)COCCn1nc(C(F)(F)F)c2c(=O)[nH]ncc21)C1CCN(c2ncc(C(F)(F)F)cn2)CC1]

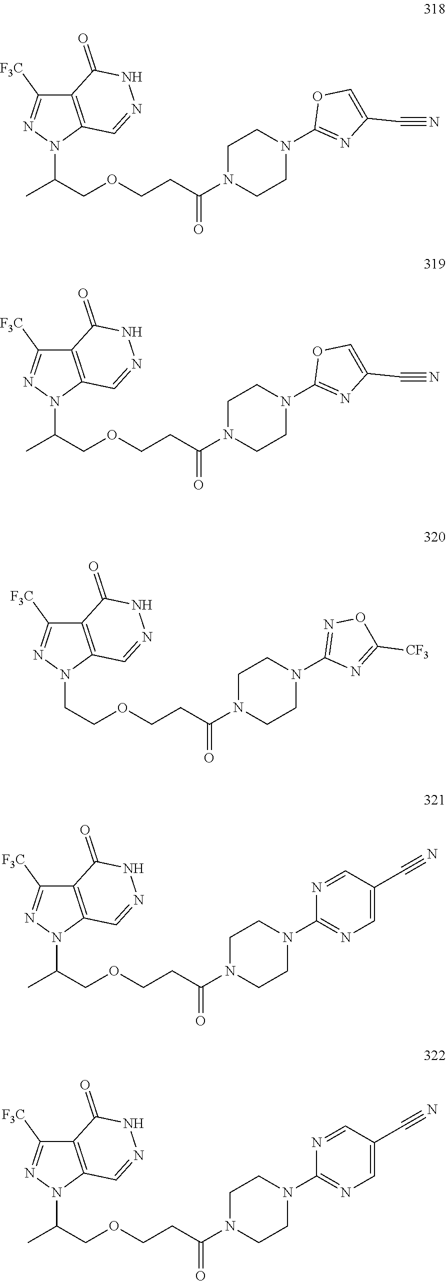 [SMILES: CC(COCCC(=O)N1CCN(c2nc(C#N)co2)CC1)n1nc(C(F)(F)F)c2c(=O)[nH]ncc21 ; CC(COCCC(=O)N1CCN(c2nc(C#N)co2)CC1)n1nc(C(F)(F)F)c2c(=O)[nH]ncc21 ; O=C(CCOCCn1nc(C(F)(F)F)c2c(=O)[nH]ncc21)N1CCN(c2noc(C(F)(F)F)n2)CC1 ; CC(COCCC(=O)N1CCN(c2ncc(C#N)cn2)CC1)n1nc(C(F)(F)F)c2c(=O)[nH]ncc21 ; CC(COCCC(=O)N1CCN(c2ncc(C#N)cn2)CC1)n1nc(C(F)(F)F)c2c(=O)[nH]ncc21]

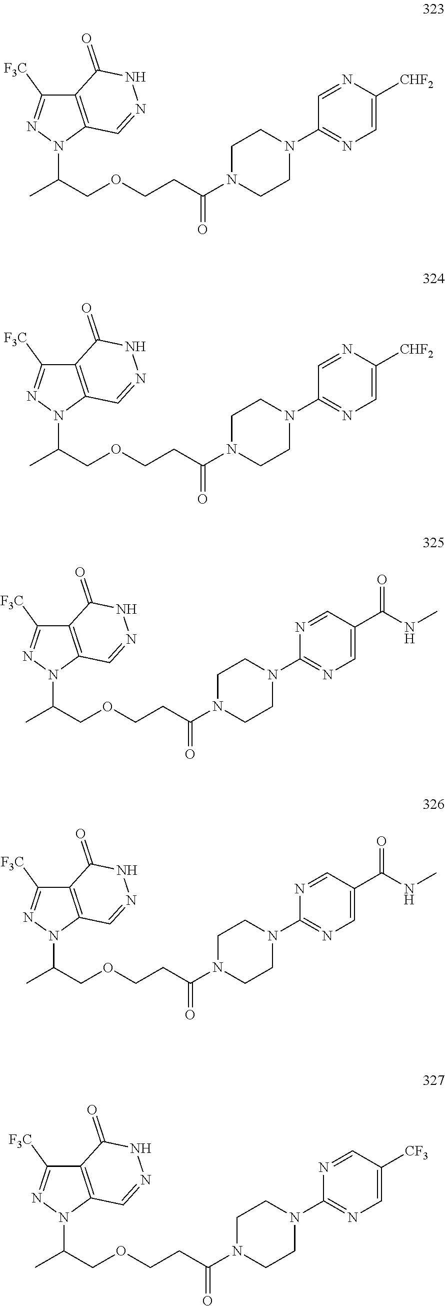 [SMILES: CC(COCCC(=O)N1CCN(c2cnc(C(F)F)cn2)CC1)n1nc(C(F)(F)F)c2c(=O)[nH]ncc21 ; CC(COCCC(=O)N1CCN(c2cnc(C(F)F)cn2)CC1)n1nc(C(F)(F)F)c2c(=O)[nH]ncc21 ; CNC(=O)c1cnc(N2CCN(C(=O)CCOCC(C)n3nc(C(F)(F)F)c4c(=O)[nH]ncc43)CC2)nc1 ; CNC(=O)c1cnc(N2CCN(C(=O)CCOCC(C)n3nc(C(F)(F)F)c4c(=O)[nH]ncc43)CC2)nc1 ; CC(COCCC(=O)N1CCN(c2ncc(C(F)(F)F)cn2)CC1)n1nc(C(F)(F)F)c2c(=O)[nH]ncc21]

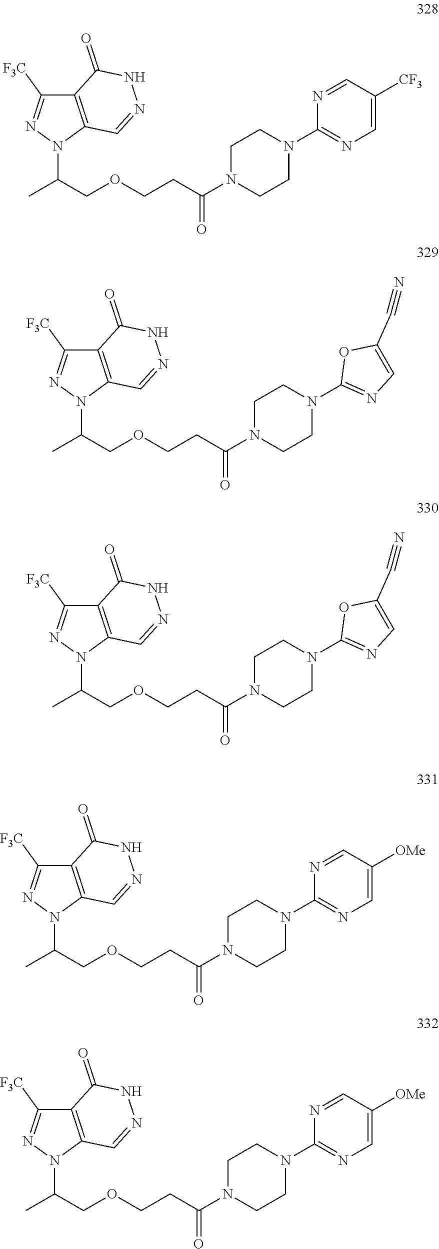 [SMILES: CC(COCCC(=O)N1CCN(c2ncc(C(F)(F)F)cn2)CC1)n1nc(C(F)(F)F)c2c(=O)[nH]ncc21 ; CC(COCCC(=O)N1CCN(c2ncc(C#N)o2)CC1)n1nc(C(F)(F)F)c2c(=O)[nH]ncc21 ; CC(COCCC(=O)N1CCN(c2ncc(C#N)o2)CC1)n1nc(C(F)(F)F)c2c(=O)[nH]ncc21 ; COc1cnc(N2CCN(C(=O)CCOCC(C)n3nc(C(F)(F)F)c4c(=O)[nH]ncc43)CC2)nc1 ; COc1cnc(N2CCN(C(=O)CCOCC(C)n3nc(C(F)(F)F)c4c(=O)[nH]ncc43)CC2)nc1]

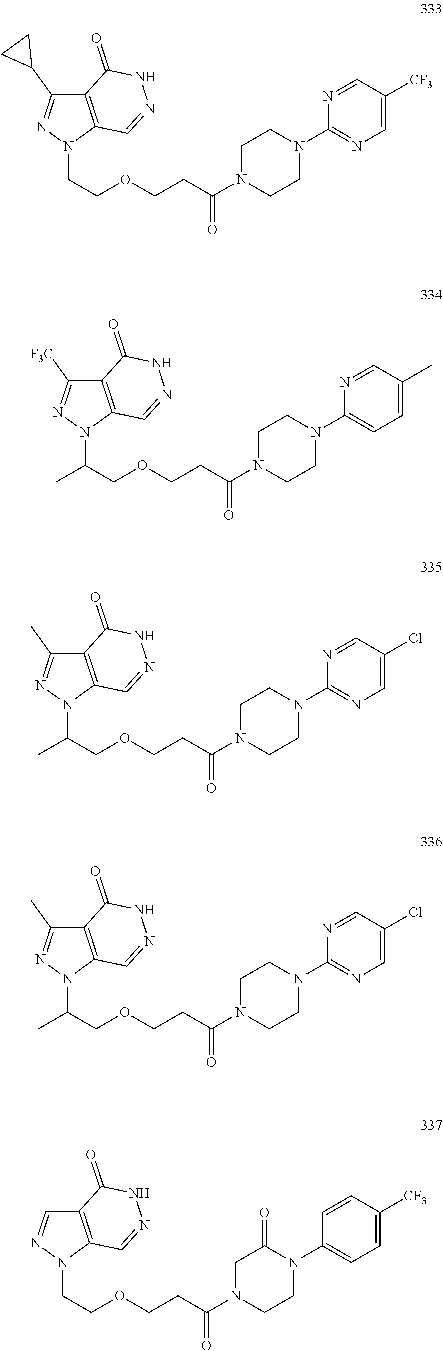

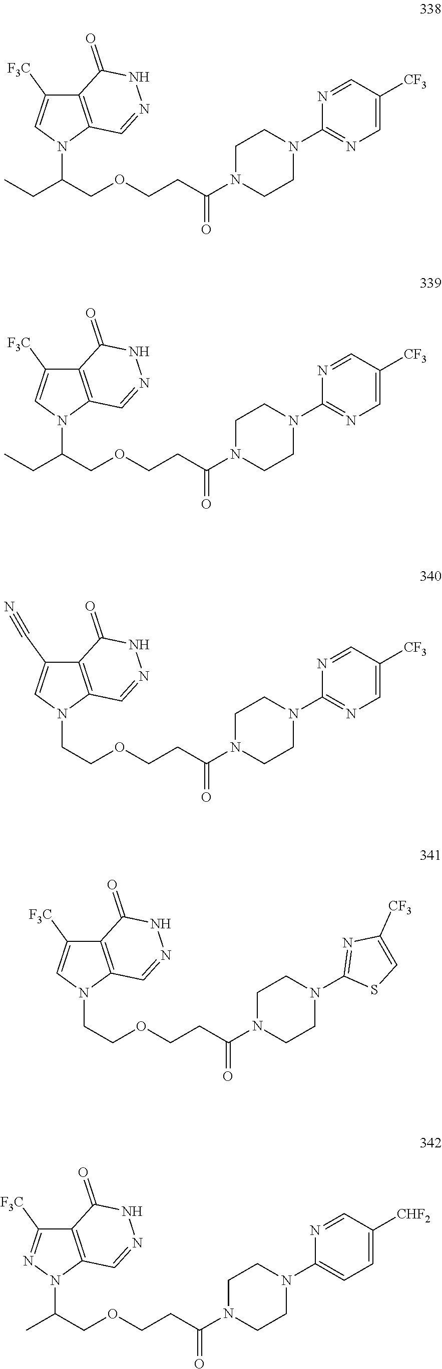 [SMILES: CCC(COCCC(=O)N1CCN(c2ncc(C(F)(F)F)cn2)CC1)n1cc(C(F)(F)F)c2c(=O)[nH]ncc21 ; CCC(COCCC(=O)N1CCN(c2ncc(C(F)(F)F)cn2)CC1)n1cc(C(F)(F)F)c2c(=O)[nH]ncc21 ; N#Cc1cn(CCOCCC(=O)N2CCN(c3ncc(C(F)(F)F)cn3)CC2)c2cn[nH]c(=O)c12 ; O=C(CCOCCn1cc(C(F)(F)F)c2c(=O)[nH]ncc21)N1CCN(c2nc(C(F)(F)F)cs2)CC1 ; CC(COCCC(=O)N1CCN(c2ccc(C(F)F)cn2)CC1)n1nc(C(F)(F)F)c2c(=O)[nH]ncc21]

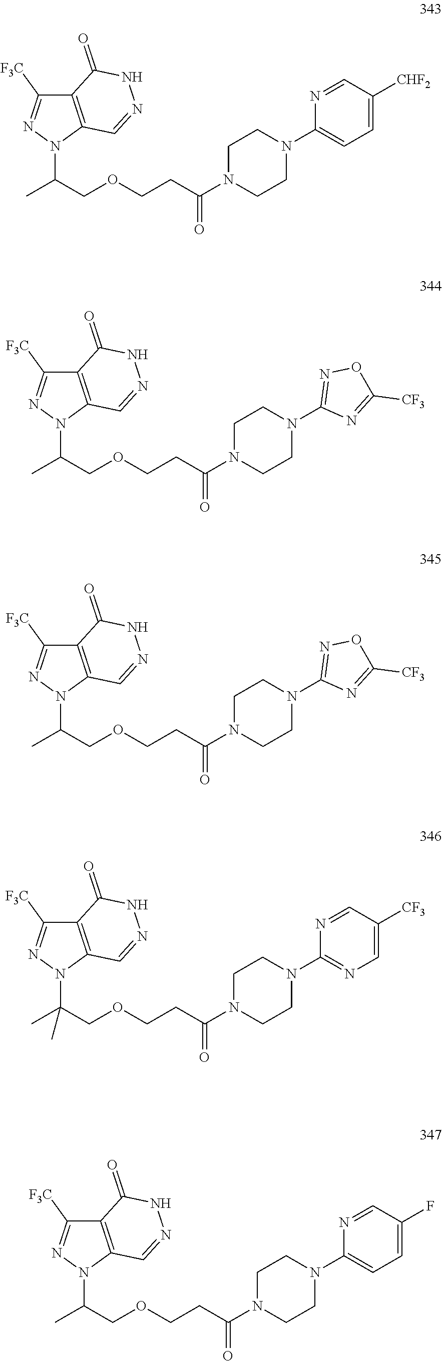 [SMILES: CC(COCCC(=O)N1CCN(c2ccc(C(F)F)cn2)CC1)n1nc(C(F)(F)F)c2c(=O)[nH]ncc21 ; CC(COCCC(=O)N1CCN(c2noc(C(F)(F)F)n2)CC1)n1nc(C(F)(F)F)c2c(=O)[nH]ncc21 ; CC(COCCC(=O)N1CCN(c2noc(C(F)(F)F)n2)CC1)n1nc(C(F)(F)F)c2c(=O)[nH]ncc21 ; CC(C)(COCCC(=O)N1CCN(c2ncc(C(F)(F)F)cn2)CC1)n1nc(C(F)(F)F)c2c(=O)[nH]ncc21 ; CC(COCCC(=O)N1CCN(c2ccc(F)cn2)CC1)n1nc(C(F)(F)F)c2c(=O)[nH]ncc21]

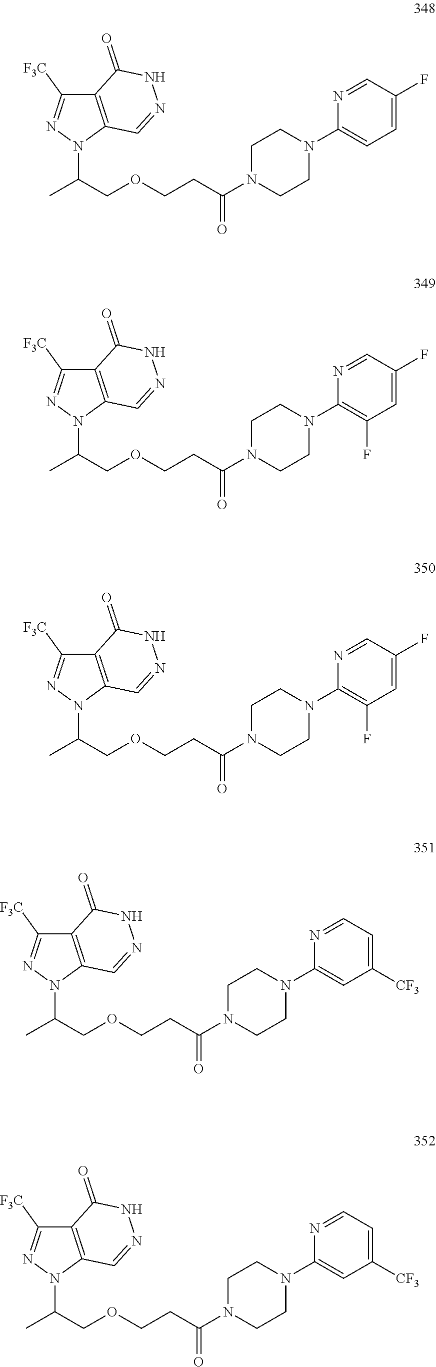 [SMILES: CC(COCCC(=O)N1CCN(c2ccc(F)cn2)CC1)n1nc(C(F)(F)F)c2c(=O)[nH]ncc21 ; CC(COCCC(=O)N1CCN(c2ncc(F)cc2F)CC1)n1nc(C(F)(F)F)c2c(=O)[nH]ncc21 ; CC(COCCC(=O)N1CCN(c2ncc(F)cc2F)CC1)n1nc(C(F)(F)F)c2c(=O)[nH]ncc21 ; CC(COCCC(=O)N1CCN(c2cc(C(F)(F)F)ccn2)CC1)n1nc(C(F)(F)F)c2c(=O)[nH]ncc21 ; CC(COCCC(=O)N1CCN(c2cc(C(F)(F)F)ccn2)CC1)n1nc(C(F)(F)F)c2c(=O)[nH]ncc21]

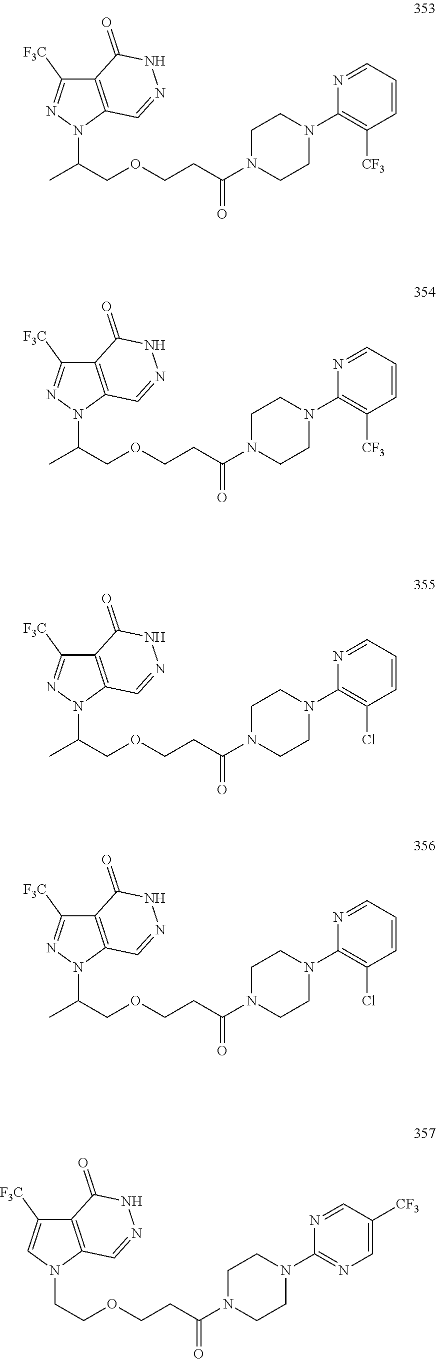 [SMILES: CC(COCCC(=O)N1CCN(c2ncccc2C(F)(F)F)CC1)n1nc(C(F)(F)F)c2c(=O)[nH]ncc21 ; CC(COCCC(=O)N1CCN(c2ncccc2C(F)(F)F)CC1)n1nc(C(F)(F)F)c2c(=O)[nH]ncc21 ; CC(COCCC(=O)N1CCN(c2ncccc2Cl)CC1)n1nc(C(F)(F)F)c2c(=O)[nH]ncc21 ; CC(COCCC(=O)N1CCN(c2ncccc2Cl)CC1)n1nc(C(F)(F)F)c2c(=O)[nH]ncc21 ; O=C(CCOCCn1cc(C(F)(F)F)c2c(=O)[nH]ncc21)N1CCN(c2ncc(C(F)(F)F)cn2)CC1]

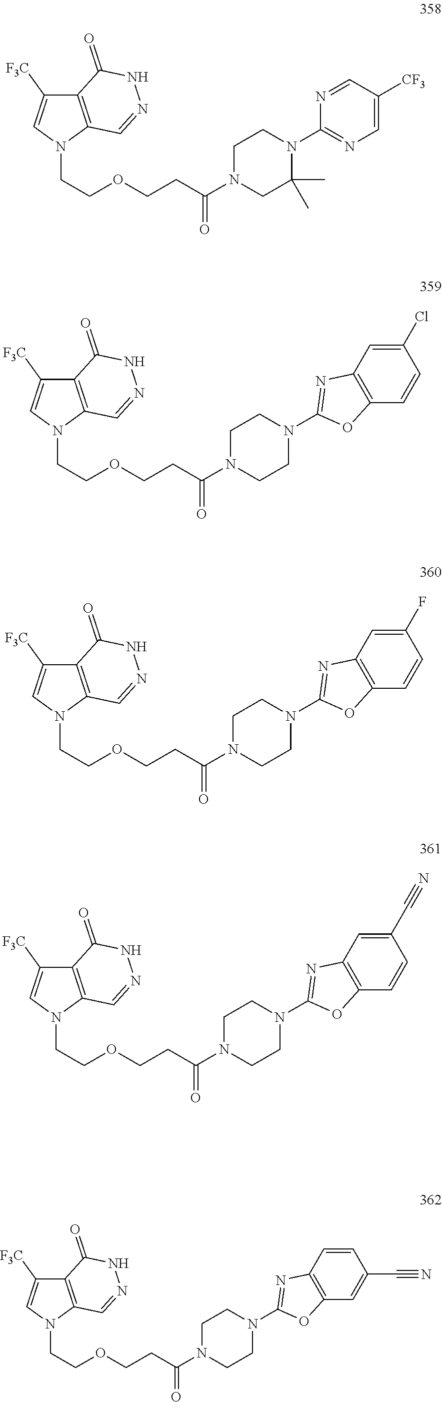 [SMILES: CC1(C)CN(C(=O)CCOCCn2cc(C(F)(F)F)c3c(=O)[nH]ncc32)CCN1c1ncc(C(F)(F)F)cn1 ; O=C(CCOCCn1cc(C(F)(F)F)c2c(=O)[nH]ncc21)N1CCN(c2nc3cc(Cl)ccc3o2)CC1 ; O=C(CCOCCn1cc(C(F)(F)F)c2c(=O)[nH]ncc21)N1CCN(c2nc3cc(F)ccc3o2)CC1 ; N#Cc1ccc2oc(N3CCN(C(=O)CCOCCn4cc(C(F)(F)F)c5c(=O)[nH]ncc54)CC3)nc2c1 ; N#Cc1ccc2nc(N3CCN(C(=O)CCOCCn4cc(C(F)(F)F)c5c(=O)[nH]ncc54)CC3)oc2c1]

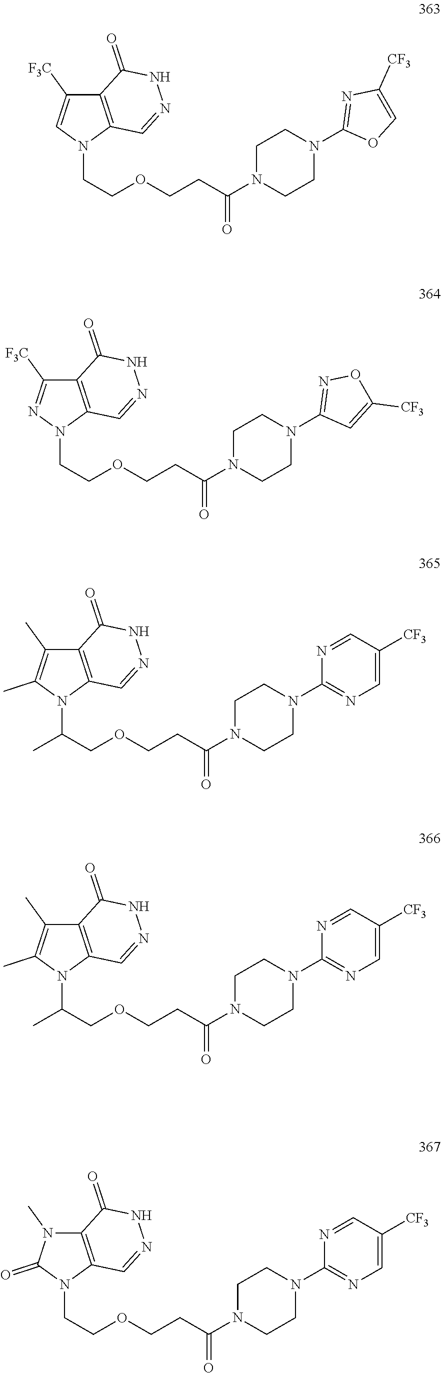 [SMILES: O=C(CCOCCn1cc(C(F)(F)F)c2c(=O)[nH]ncc21)N1CCN(c2nc(C(F)(F)F)co2)CC1 ; O=C(CCOCCn1nc(C(F)(F)F)c2c(=O)[nH]ncc21)N1CCN(c2cc(C(F)(F)F)on2)CC1 ; Cc1c(C)n(C(C)COCCC(=O)N2CCN(c3ncc(C(F)(F)F)cn3)CC2)c2cn[nH]c(=O)c12 ; Cc1c(C)n(C(C)COCCC(=O)N2CCN(c3ncc(C(F)(F)F)cn3)CC2)c2cn[nH]c(=O)c12 ; Cn1c(=O)n(CCOCCC(=O)N2CCN(c3ncc(C(F)(F)F)cn3)CC2)c2cn[nH]c(=O)c21]

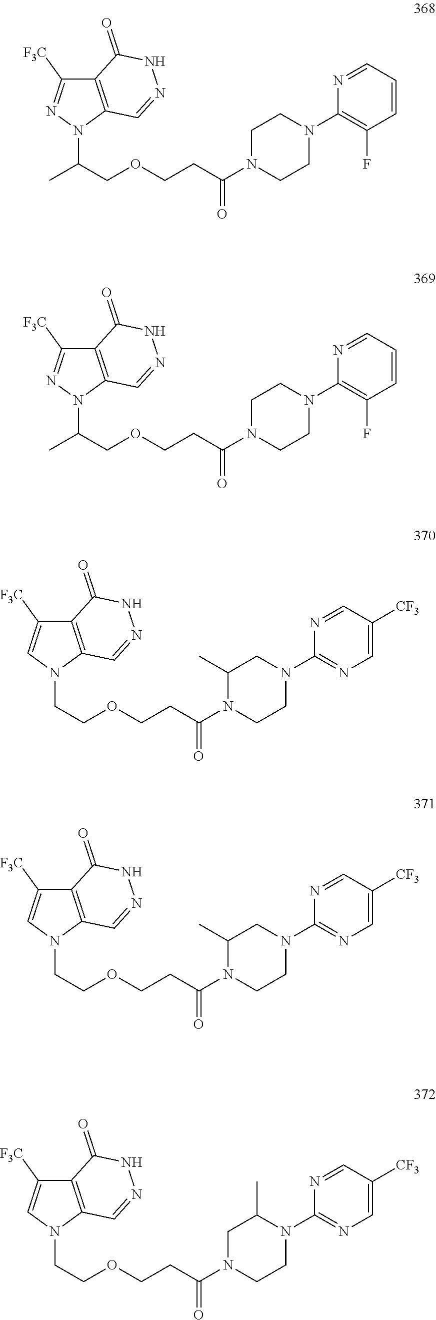 [SMILES: CC(COCCC(=O)N1CCN(c2ncccc2F)CC1)n1nc(C(F)(F)F)c2c(=O)[nH]ncc21 ; CC(COCCC(=O)N1CCN(c2ncccc2F)CC1)n1nc(C(F)(F)F)c2c(=O)[nH]ncc21 ; CC1CN(c2ncc(C(F)(F)F)cn2)CCN1C(=O)CCOCCn1cc(C(F)(F)F)c2c(=O)[nH]ncc21 ; CC1CN(c2ncc(C(F)(F)F)cn2)CCN1C(=O)CCOCCn1cc(C(F)(F)F)c2c(=O)[nH]ncc21 ; CC1CN(C(=O)CCOCCn2cc(C(F)(F)F)c3c(=O)[nH]ncc32)CCN1c1ncc(C(F)(F)F)cn1]

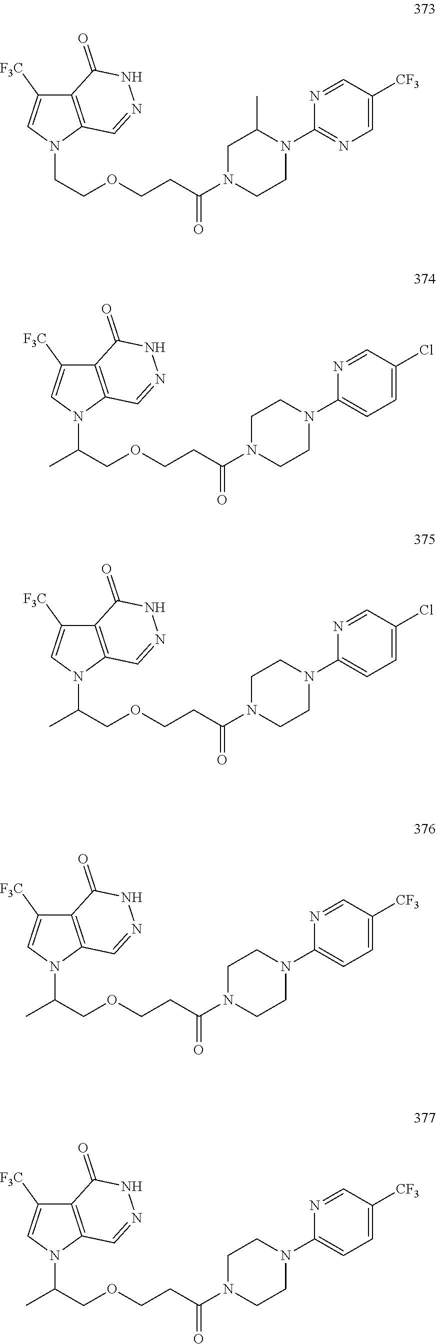 [SMILES: CC1CN(C(=O)CCOCCn2cc(C(F)(F)F)c3c(=O)[nH]ncc32)CCN1c1ncc(C(F)(F)F)cn1 ; CC(COCCC(=O)N1CCN(c2ccc(Cl)cn2)CC1)n1cc(C(F)(F)F)c2c(=O)[nH]ncc21 ; CC(COCCC(=O)N1CCN(c2ccc(Cl)cn2)CC1)n1cc(C(F)(F)F)c2c(=O)[nH]ncc21 ; CC(COCCC(=O)N1CCN(c2ccc(C(F)(F)F)cn2)CC1)n1cc(C(F)(F)F)c2c(=O)[nH]ncc21 ; CC(COCCC(=O)N1CCN(c2ccc(C(F)(F)F)cn2)CC1)n1cc(C(F)(F)F)c2c(=O)[nH]ncc21]

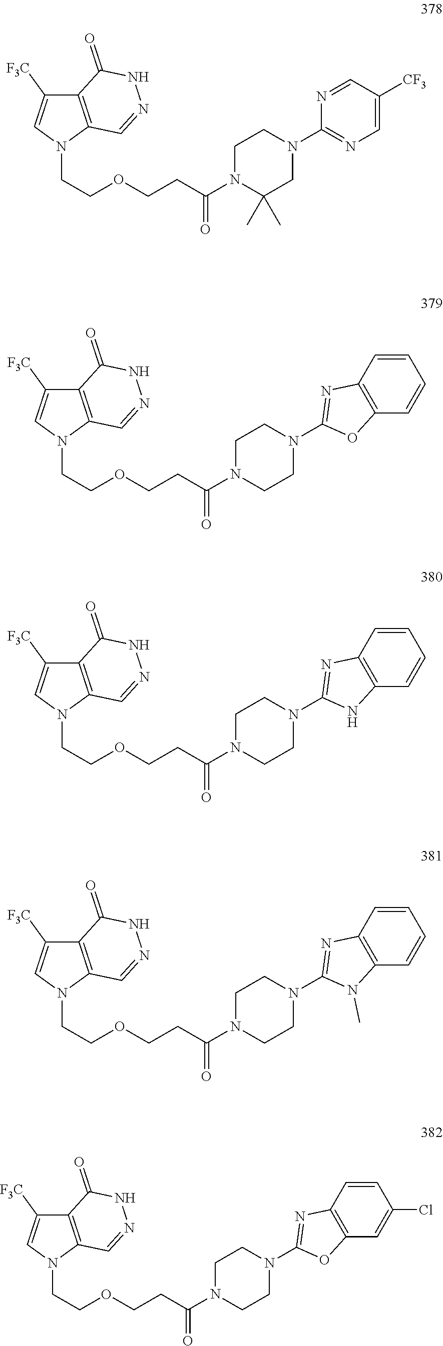 [SMILES: CC1(C)CN(c2ncc(C(F)(F)F)cn2)CCN1C(=O)CCOCCn1cc(C(F)(F)F)c2c(=O)[nH]ncc21 ; O=C(CCOCCn1cc(C(F)(F)F)c2c(=O)[nH]ncc21)N1CCN(c2nc3ccccc3o2)CC1 ; O=C(CCOCCn1cc(C(F)(F)F)c2c(=O)[nH]ncc21)N1CCN(c2nc3ccccc3[nH]2)CC1 ; Cn1c(N2CCN(C(=O)CCOCCn3cc(C(F)(F)F)c4c(=O)[nH]ncc43)CC2)nc2ccccc21 ; O=C(CCOCCn1cc(C(F)(F)F)c2c(=O)[nH]ncc21)N1CCN(c2nc3ccc(Cl)cc3o2)CC1]

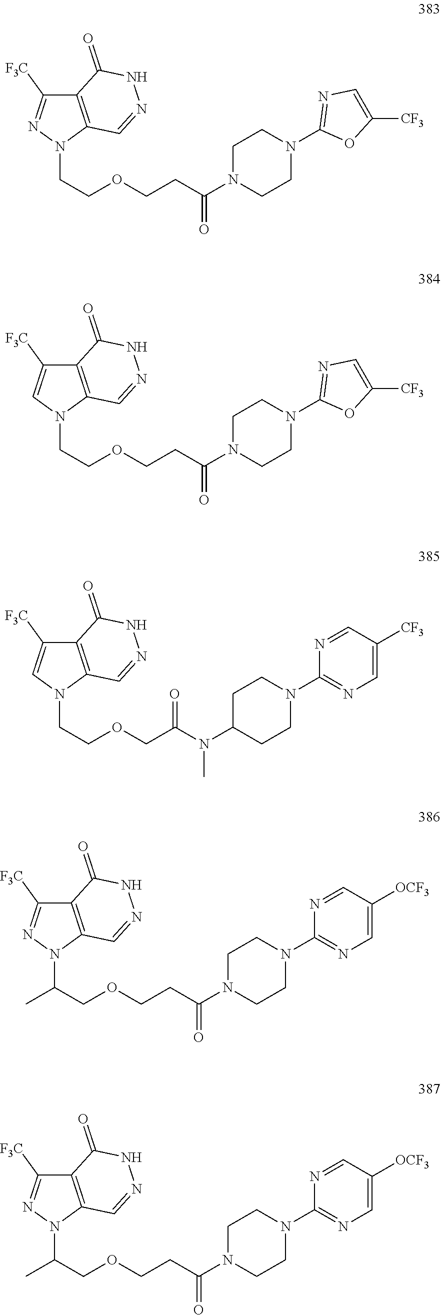 [SMILES: O=C(CCOCCn1nc(C(F)(F)F)c2c(=O)[nH]ncc21)N1CCN(c2ncc(C(F)(F)F)o2)CC1 ; O=C(CCOCCn1cc(C(F)(F)F)c2c(=O)[nH]ncc21)N1CCN(c2ncc(C(F)(F)F)o2)CC1 ; CN(C(=O)COCCn1cc(C(F)(F)F)c2c(=O)[nH]ncc21)C1CCN(c2ncc(C(F)(F)F)cn2)CC1 ; CC(COCCC(=O)N1CCN(c2ncc(OC(F)(F)F)cn2)CC1)n1nc(C(F)(F)F)c2c(=O)[nH]ncc21 ; CC(COCCC(=O)N1CCN(c2ncc(OC(F)(F)F)cn2)CC1)n1nc(C(F)(F)F)c2c(=O)[nH]ncc21]

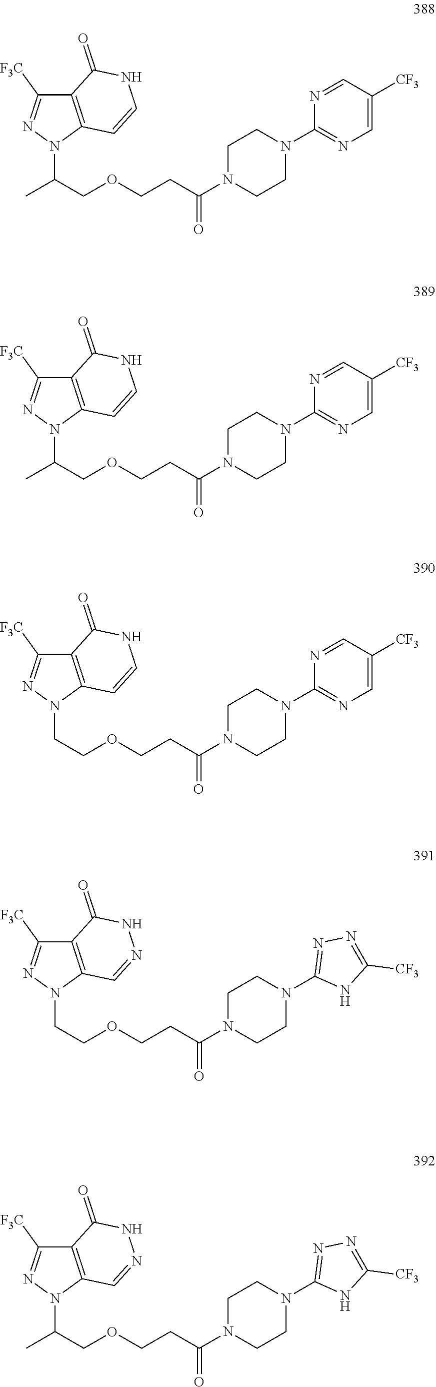 [SMILES: CC(COCCC(=O)N1CCN(c2ncc(C(F)(F)F)cn2)CC1)n1nc(C(F)(F)F)c2c(=O)[nH]ccc21 ; CC(COCCC(=O)N1CCN(c2ncc(C(F)(F)F)cn2)CC1)n1nc(C(F)(F)F)c2c(=O)[nH]ccc21 ; O=C(CCOCCn1nc(C(F)(F)F)c2c(=O)[nH]ccc21)N1CCN(c2ncc(C(F)(F)F)cn2)CC1 ; O=C(CCOCCn1nc(C(F)(F)F)c2c(=O)[nH]ncc21)N1CCN(c2nnc(C(F)(F)F)[nH]2)CC1 ; CC(COCCC(=O)N1CCN(c2nnc(C(F)(F)F)[nH]2)CC1)n1nc(C(F)(F)F)c2c(=O)[nH]ncc21]

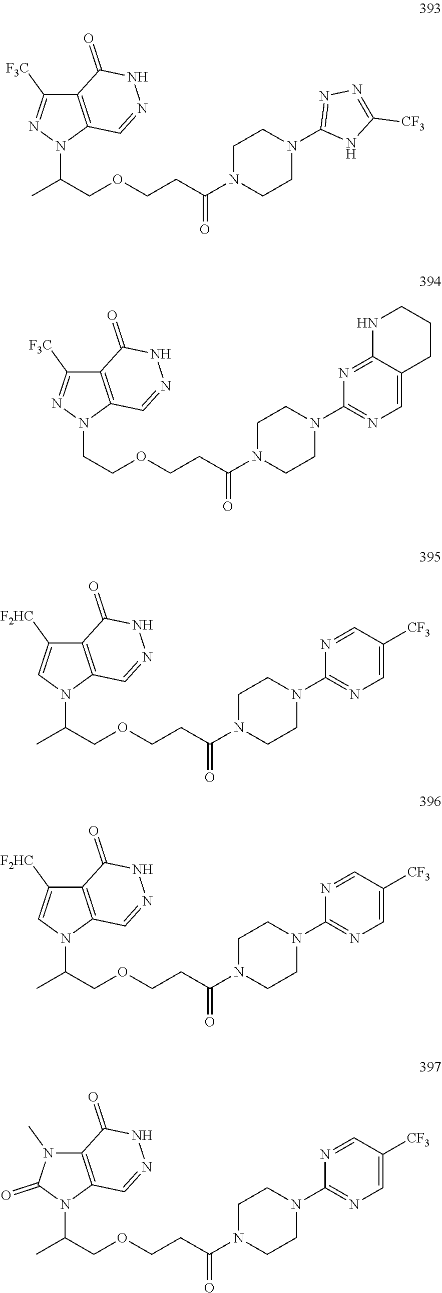 [SMILES: CC(COCCC(=O)N1CCN(c2nnc(C(F)(F)F)[nH]2)CC1)n1nc(C(F)(F)F)c2c(=O)[nH]ncc21 ; O=C(CCOCCn1nc(C(F)(F)F)c2c(=O)[nH]ncc21)N1CCN(c2ncc3c(n2)NCCC3)CC1 ; CC(COCCC(=O)N1CCN(c2ncc(C(F)(F)F)cn2)CC1)n1cc(C(F)F)c2c(=O)[nH]ncc21 ; CC(COCCC(=O)N1CCN(c2ncc(C(F)(F)F)cn2)CC1)n1cc(C(F)F)c2c(=O)[nH]ncc21 ; CC(COCCC(=O)N1CCN(c2ncc(C(F)(F)F)cn2)CC1)n1c(=O)n(C)c2c(=O)[nH]ncc21]

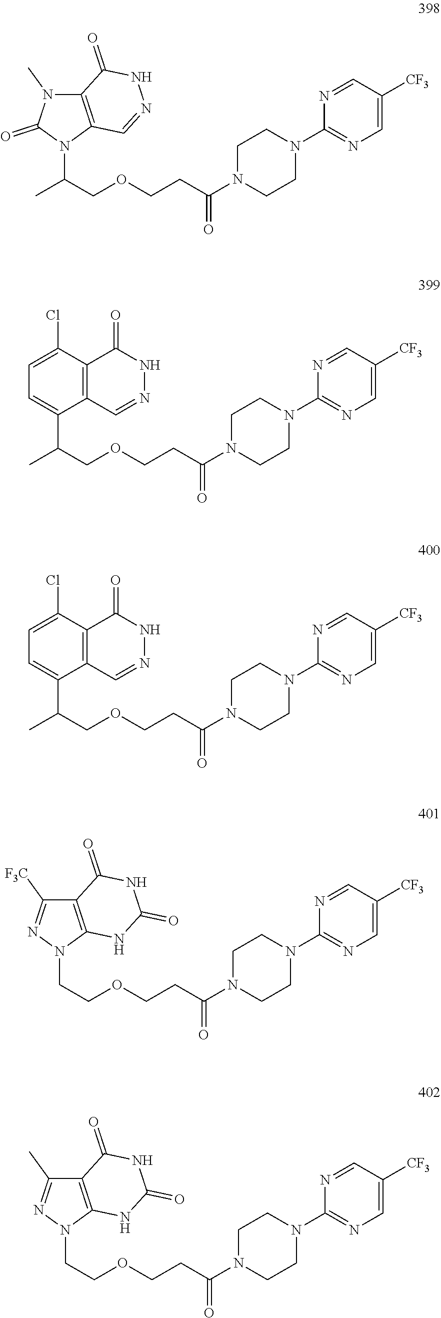 [SMILES: CC(COCCC(=O)N1CCN(c2ncc(C(F)(F)F)cn2)CC1)n1c(=O)n(C)c2c(=O)[nH]ncc21 ; CC(COCCC(=O)N1CCN(c2ncc(C(F)(F)F)cn2)CC1)c1ccc(Cl)c2c(=O)[nH]ncc12 ; CC(COCCC(=O)N1CCN(c2ncc(C(F)(F)F)cn2)CC1)c1ccc(Cl)c2c(=O)[nH]ncc12 ; O=C(CCOCCn1nc(C(F)(F)F)c2c(=O)[nH]c(=O)[nH]c21)N1CCN(c2ncc(C(F)(F)F)cn2)CC1 ; Cc1nn(CCOCCC(=O)N2CCN(c3ncc(C(F)(F)F)cn3)CC2)c2[nH]c(=O)[nH]c(=O)c12]

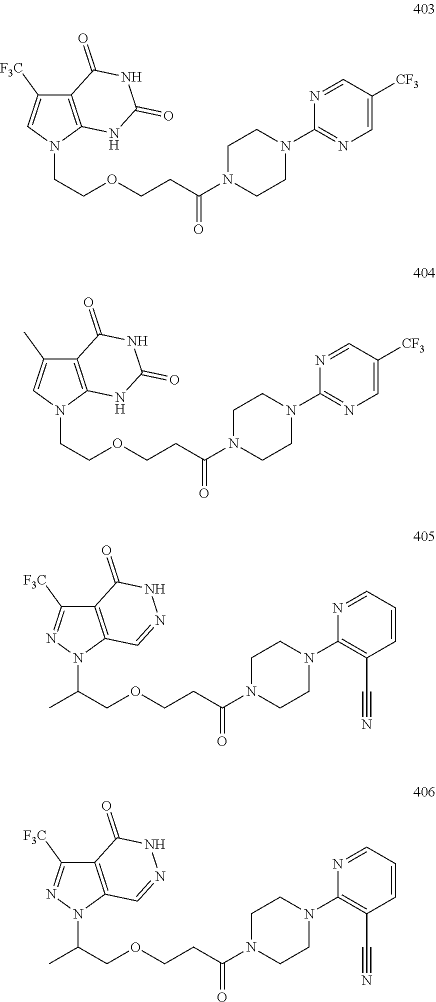 [SMILES: O=C(CCOCCn1cc(C(F)(F)F)c2c(=O)[nH]c(=O)[nH]c21)N1CCN(c2ncc(C(F)(F)F)cn2)CC1 ; Cc1cn(CCOCCC(=O)N2CCN(c3ncc(C(F)(F)F)cn3)CC2)c2[nH]c(=O)[nH]c(=O)c12 ; CC(COCCC(=O)N1CCN(c2ncccc2C#N)CC1)n1nc(C(F)(F)F)c2c(=O)[nH]ncc21 ; CC(COCCC(=O)N1CCN(c2ncccc2C#N)CC1)n1nc(C(F)(F)F)c2c(=O)[nH]ncc21]

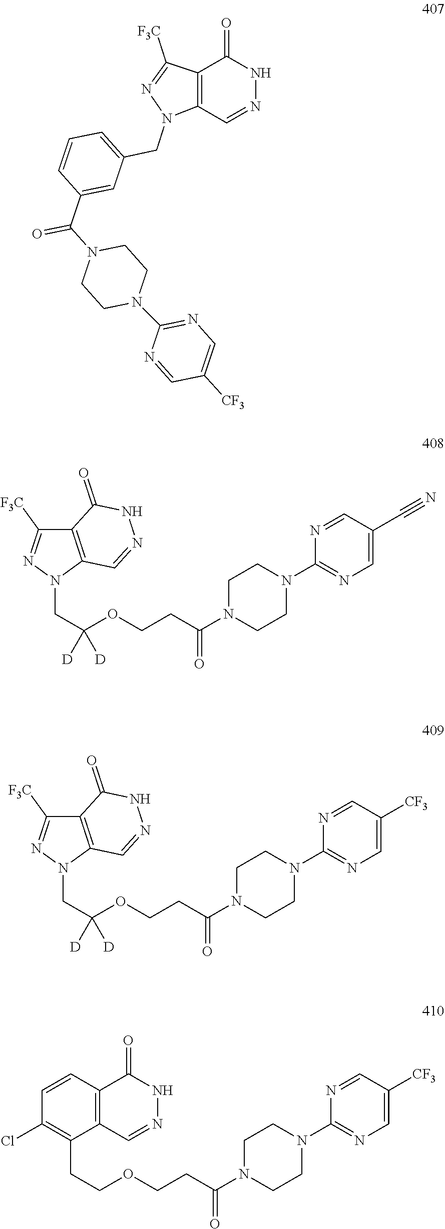 [SMILES: O=C(c1cccc(Cn2nc(C(F)(F)F)c3c(=O)[nH]ncc32)c1)N1CCN(c2ncc(C(F)(F)F)cn2)CC1 ; [2H]C([2H])(Cn1nc(C(F)(F)F)c2c(=O)[nH]ncc21)OCCC(=O)N1CCN(c2ncc(C#N)cn2)CC1 ; [2H]C([2H])(Cn1nc(C(F)(F)F)c2c(=O)[nH]ncc21)OCCC(=O)N1CCN(c2ncc(C(F)(F)F)cn2)CC1 ; O=C(CCOCCc1c(Cl)ccc2c(=O)[nH]ncc12)N1CCN(c2ncc(C(F)(F)F)cn2)CC1]

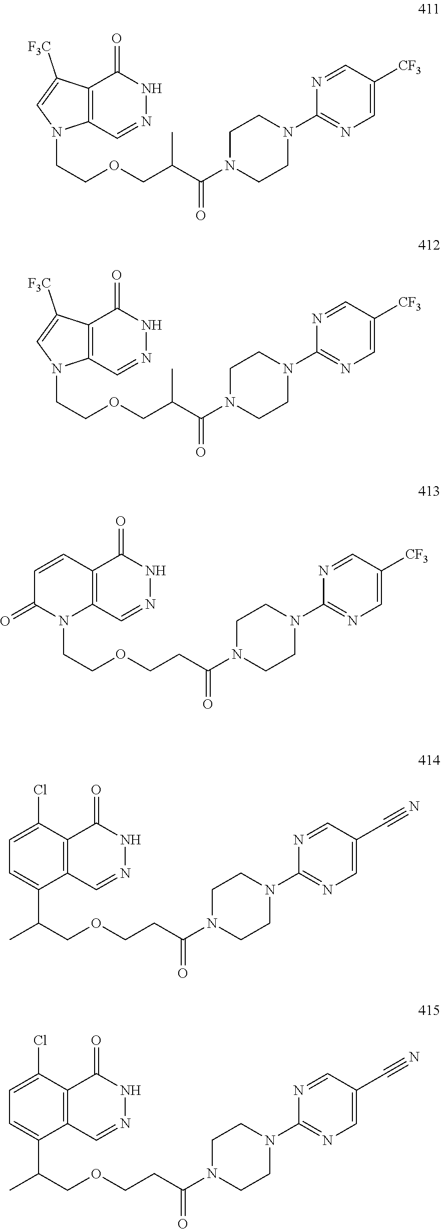 [SMILES: CC(COCCn1cc(C(F)(F)F)c2c(=O)[nH]ncc21)C(=O)N1CCN(c2ncc(C(F)(F)F)cn2)CC1 ; CC(COCCn1cc(C(F)(F)F)c2c(=O)[nH]ncc21)C(=O)N1CCN(c2ncc(C(F)(F)F)cn2)CC1 ; O=C(CCOCCn1c(=O)ccc2c(=O)[nH]ncc21)N1CCN(c2ncc(C(F)(F)F)cn2)CC1 ; CC(COCCC(=O)N1CCN(c2ncc(C#N)cn2)CC1)c1ccc(Cl)c2c(=O)[nH]ncc12 ; CC(COCCC(=O)N1CCN(c2ncc(C#N)cn2)CC1)c1ccc(Cl)c2c(=O)[nH]ncc12]

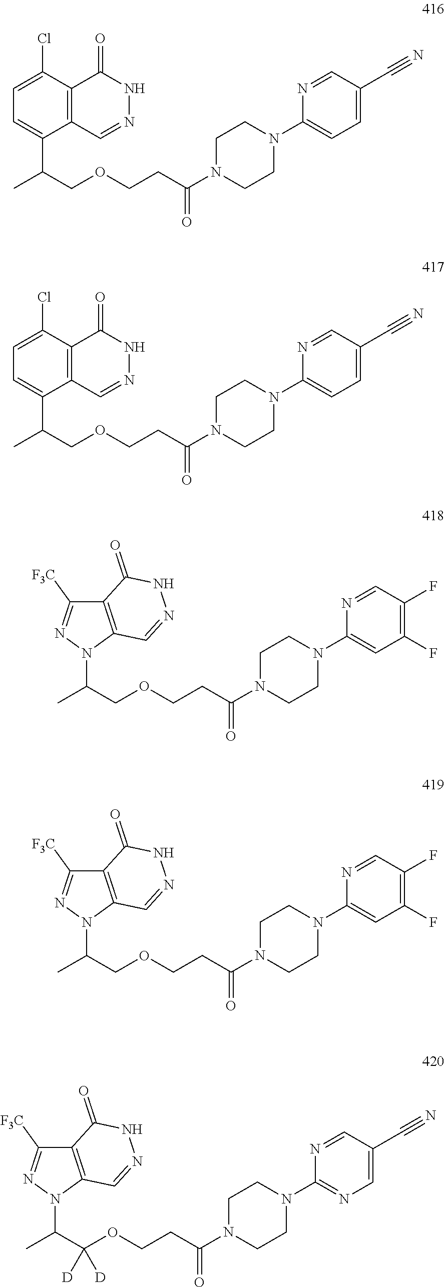 [SMILES: CC(COCCC(=O)N1CCN(c2ccc(C#N)cn2)CC1)c1ccc(Cl)c2c(=O)[nH]ncc12 ; CC(COCCC(=O)N1CCN(c2ccc(C#N)cn2)CC1)c1ccc(Cl)c2c(=O)[nH]ncc12 ; CC(COCCC(=O)N1CCN(c2cc(F)c(F)cn2)CC1)n1nc(C(F)(F)F)c2c(=O)[nH]ncc21 ; CC(COCCC(=O)N1CCN(c2cc(F)c(F)cn2)CC1)n1nc(C(F)(F)F)c2c(=O)[nH]ncc21 ; [2H]C([2H])(OCCC(=O)N1CCN(c2ncc(C#N)cn2)CC1)C(C)n1nc(C(F)(F)F)c2c(=O)[nH]ncc21]

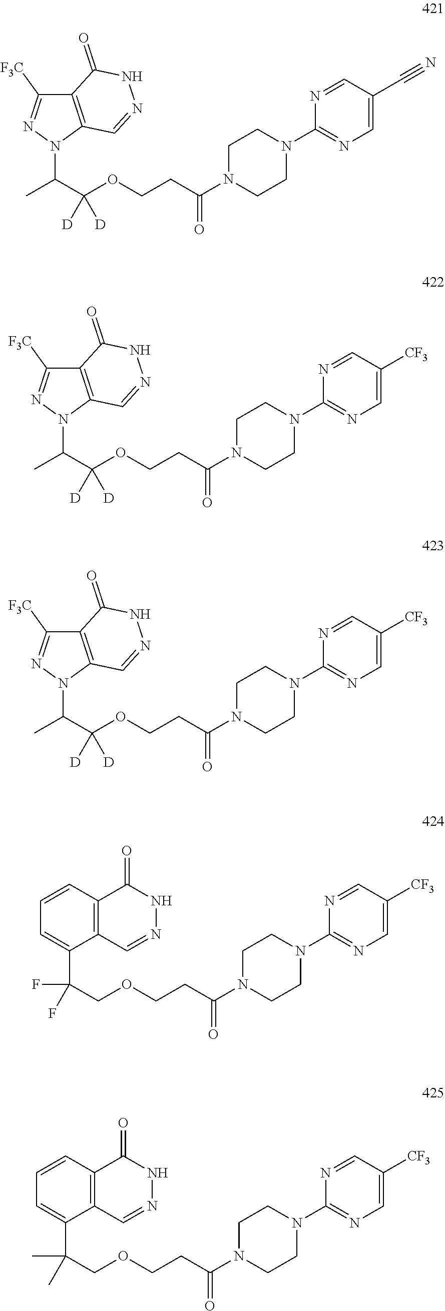 [SMILES: [2H]C([2H])(OCCC(=O)N1CCN(c2ncc(C#N)cn2)CC1)C(C)n1nc(C(F)(F)F)c2c(=O)[nH]ncc21 ; [2H]C([2H])(OCCC(=O)N1CCN(c2ncc(C(F)(F)F)cn2)CC1)C(C)n1nc(C(F)(F)F)c2c(=O)[nH]ncc21 ; [2H]C([2H])(OCCC(=O)N1CCN(c2ncc(C(F)(F)F)cn2)CC1)C(C)n1nc(C(F)(F)F)c2c(=O)[nH]ncc21 ; O=C(CCOCC(F)(F)c1cccc2c(=O)[nH]ncc12)N1CCN(c2ncc(C(F)(F)F)cn2)CC1 ; CC(C)(COCCC(=O)N1CCN(c2ncc(C(F)(F)F)cn2)CC1)c1cccc2c(=O)[nH]ncc12]

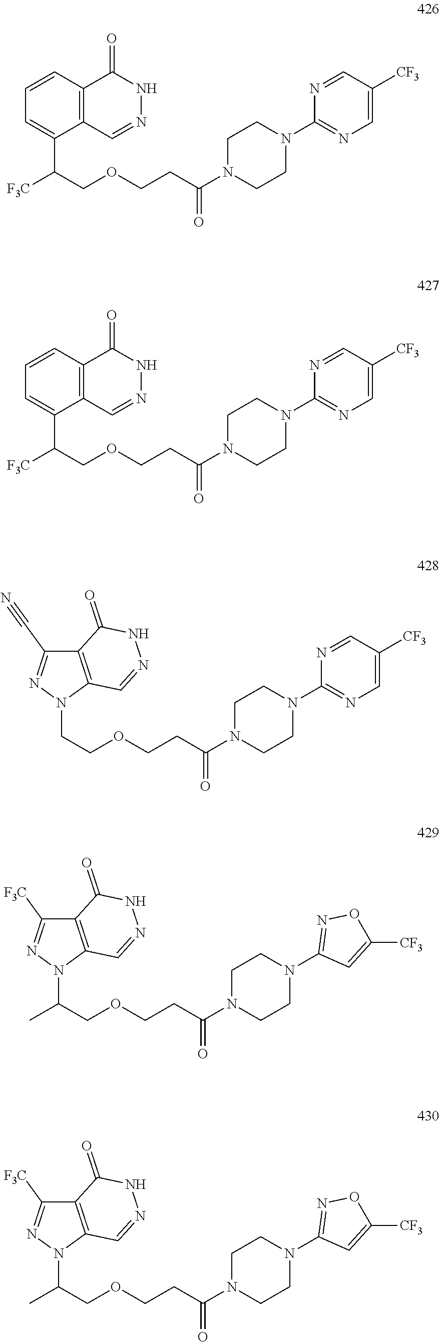 [SMILES: O=C(CCOCC(c1cccc2c(=O)[nH]ncc12)C(F)(F)F)N1CCN(c2ncc(C(F)(F)F)cn2)CC1 ; O=C(CCOCC(c1cccc2c(=O)[nH]ncc12)C(F)(F)F)N1CCN(c2ncc(C(F)(F)F)cn2)CC1 ; N#Cc1nn(CCOCCC(=O)N2CCN(c3ncc(C(F)(F)F)cn3)CC2)c2cn[nH]c(=O)c12 ; CC(COCCC(=O)N1CCN(c2cc(C(F)(F)F)on2)CC1)n1nc(C(F)(F)F)c2c(=O)[nH]ncc21 ; CC(COCCC(=O)N1CCN(c2cc(C(F)(F)F)on2)CC1)n1nc(C(F)(F)F)c2c(=O)[nH]ncc21]

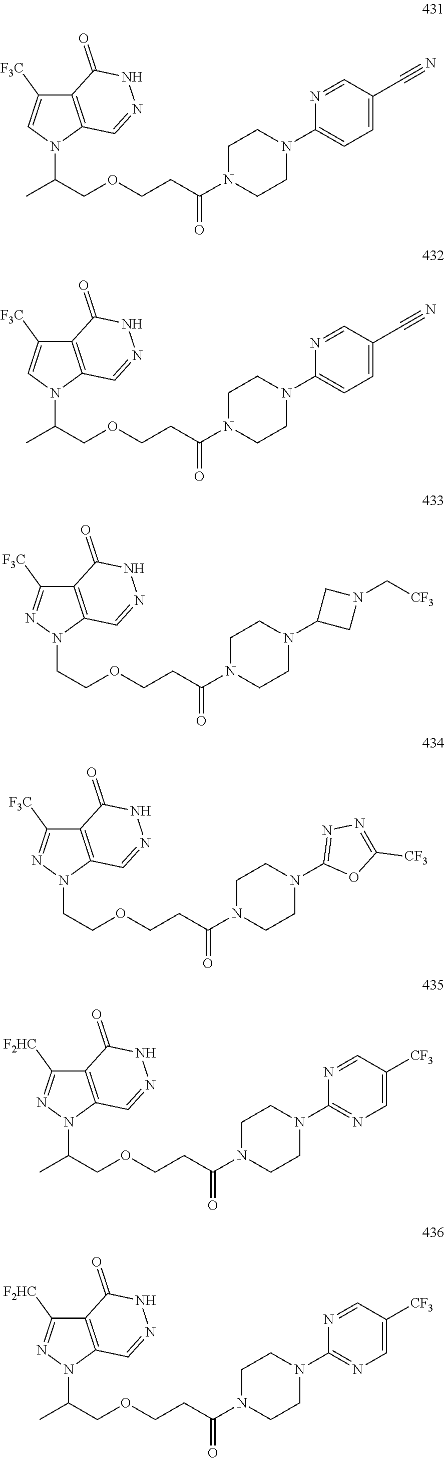 [SMILES: CC(COCCC(=O)N1CCN(c2ccc(C#N)cn2)CC1)n1cc(C(F)(F)F)c2c(=O)[nH]ncc21 ; CC(COCCC(=O)N1CCN(c2ccc(C#N)cn2)CC1)n1cc(C(F)(F)F)c2c(=O)[nH]ncc21 ; O=C(CCOCCn1nc(C(F)(F)F)c2c(=O)[nH]ncc21)N1CCN(C2CN(CC(F)(F)F)C2)CC1 ; O=C(CCOCCn1nc(C(F)(F)F)c2c(=O)[nH]ncc21)N1CCN(c2nnc(C(F)(F)F)o2)CC1 ; CC(COCCC(=O)N1CCN(c2ncc(C(F)(F)F)cn2)CC1)n1nc(C(F)F)c2c(=O)[nH]ncc21 ; CC(COCCC(=O)N1CCN(c2ncc(C(F)(F)F)cn2)CC1)n1nc(C(F)F)c2c(=O)[nH]ncc21]

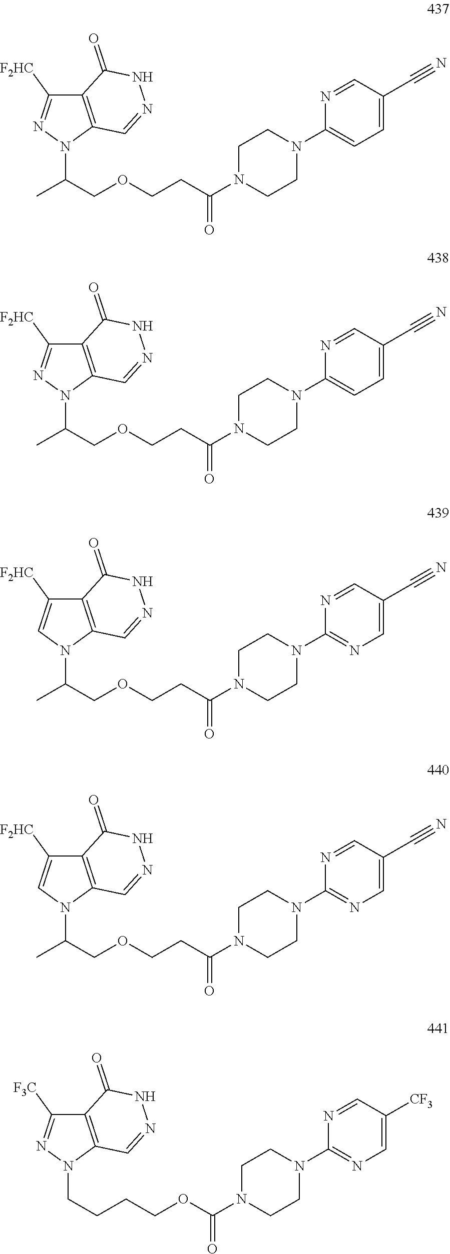 [SMILES: CC(COCCC(=O)N1CCN(c2ccc(C#N)cn2)CC1)n1nc(C(F)F)c2c(=O)[nH]ncc21 ; CC(COCCC(=O)N1CCN(c2ccc(C#N)cn2)CC1)n1nc(C(F)F)c2c(=O)[nH]ncc21 ; CC(COCCC(=O)N1CCN(c2ncc(C#N)cn2)CC1)n1cc(C(F)F)c2c(=O)[nH]ncc21 ; CC(COCCC(=O)N1CCN(c2ncc(C#N)cn2)CC1)n1cc(C(F)F)c2c(=O)[nH]ncc21 ; O=C(OCCCCn1nc(C(F)(F)F)c2c(=O)[nH]ncc21)N1CCN(c2ncc(C(F)(F)F)cn2)CC1]

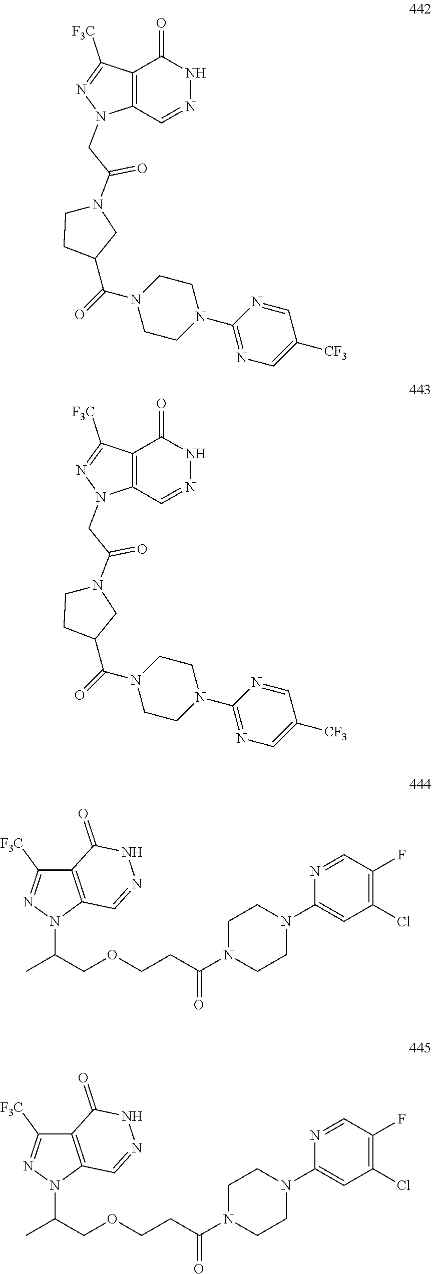 [SMILES: O=C(Cn1nc(C(F)(F)F)c2c(=O)[nH]ncc21)N1CCC(C(=O)N2CCN(c3ncc(C(F)(F)F)cn3)CC2)C1 ; O=C(Cn1nc(C(F)(F)F)c2c(=O)[nH]ncc21)N1CCC(C(=O)N2CCN(c3ncc(C(F)(F)F)cn3)CC2)C1 ; CC(COCCC(=O)N1CCN(c2cc(Cl)c(F)cn2)CC1)n1nc(C(F)(F)F)c2c(=O)[nH]ncc21 ; CC(COCCC(=O)N1CCN(c2cc(Cl)c(F)cn2)CC1)n1nc(C(F)(F)F)c2c(=O)[nH]ncc21]

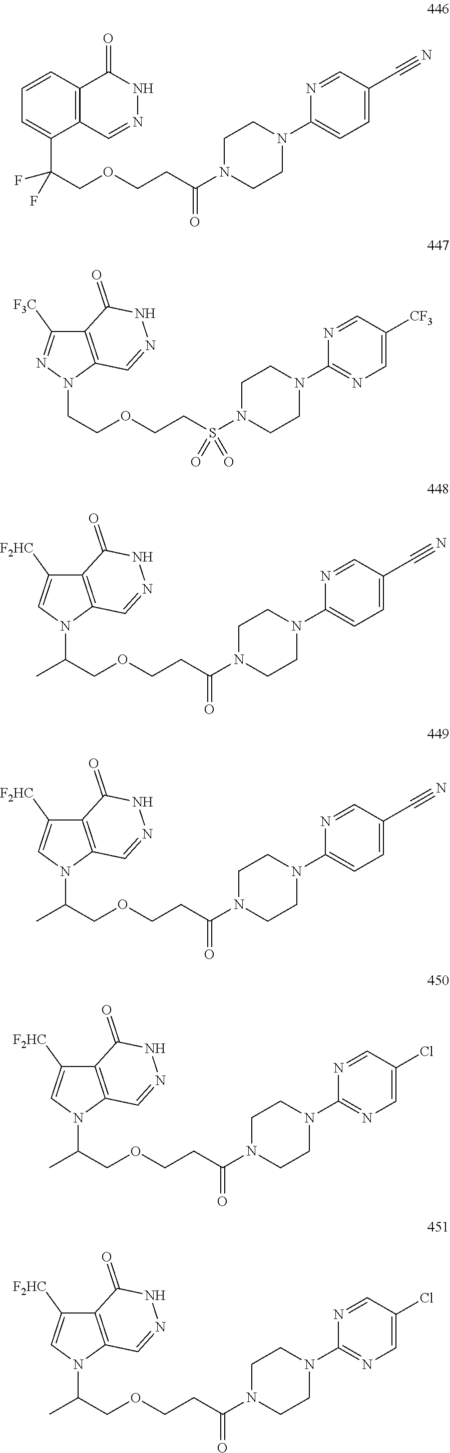 [SMILES: N#Cc1ccc(N2CCN(C(=O)CCOCC(F)(F)c3cccc4c(=O)[nH]ncc34)CC2)nc1 ; O=c1[nH]ncc2c1c(C(F)(F)F)nn2CCOCCS(=O)(=O)N1CCN(c2ncc(C(F)(F)F)cn2)CC1 ; CC(COCCC(=O)N1CCN(c2ccc(C#N)cn2)CC1)n1cc(C(F)F)c2c(=O)[nH]ncc21 ; CC(COCCC(=O)N1CCN(c2ccc(C#N)cn2)CC1)n1cc(C(F)F)c2c(=O)[nH]ncc21 ; CC(COCCC(=O)N1CCN(c2ncc(Cl)cn2)CC1)n1cc(C(F)F)c2c(=O)[nH]ncc21 ; CC(COCCC(=O)N1CCN(c2ncc(Cl)cn2)CC1)n1cc(C(F)F)c2c(=O)[nH]ncc21]

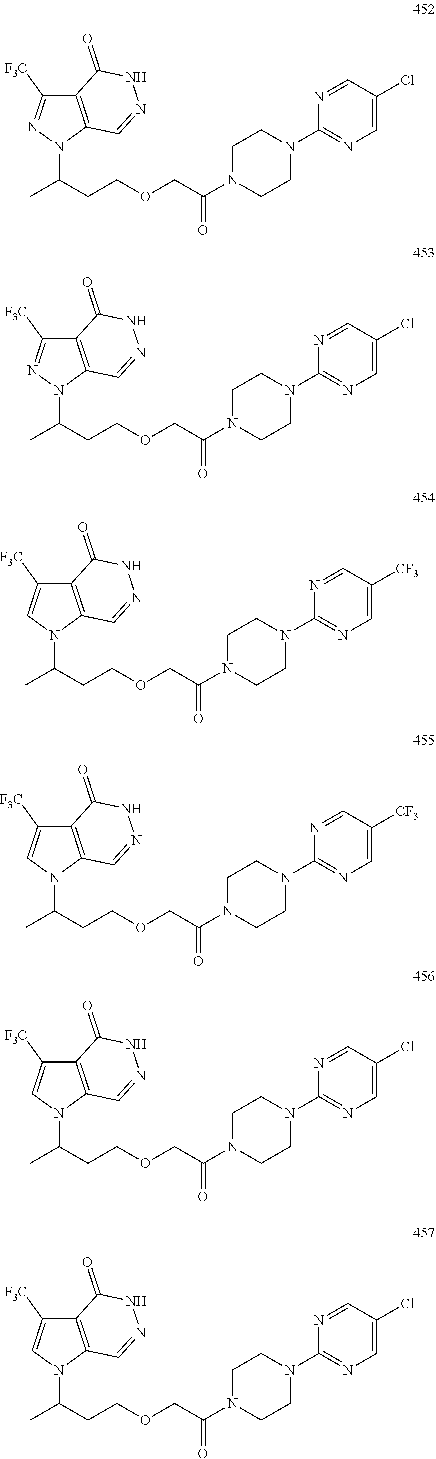 [SMILES: CC(CCOCC(=O)N1CCN(c2ncc(Cl)cn2)CC1)n1nc(C(F)(F)F)c2c(=O)[nH]ncc21 ; CC(CCOCC(=O)N1CCN(c2ncc(Cl)cn2)CC1)n1nc(C(F)(F)F)c2c(=O)[nH]ncc21 ; CC(CCOCC(=O)N1CCN(c2ncc(C(F)(F)F)cn2)CC1)n1cc(C(F)(F)F)c2c(=O)[nH]ncc21 ; CC(CCOCC(=O)N1CCN(c2ncc(C(F)(F)F)cn2)CC1)n1cc(C(F)(F)F)c2c(=O)[nH]ncc21 ; CC(CCOCC(=O)N1CCN(c2ncc(Cl)cn2)CC1)n1cc(C(F)(F)F)c2c(=O)[nH]ncc21 ; CC(CCOCC(=O)N1CCN(c2ncc(Cl)cn2)CC1)n1cc(C(F)(F)F)c2c(=O)[nH]ncc21]

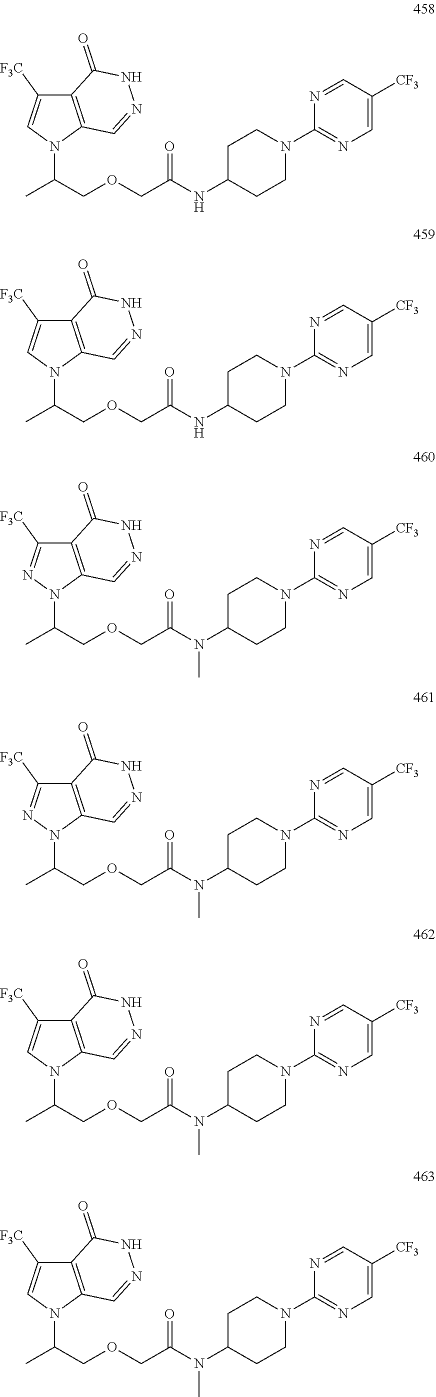 [SMILES: CC(COCC(=O)NC1CCN(c2ncc(C(F)(F)F)cn2)CC1)n1cc(C(F)(F)F)c2c(=O)[nH]ncc21 ; CC(COCC(=O)NC1CCN(c2ncc(C(F)(F)F)cn2)CC1)n1cc(C(F)(F)F)c2c(=O)[nH]ncc21 ; CC(COCC(=O)N(C)C1CCN(c2ncc(C(F)(F)F)cn2)CC1)n1nc(C(F)(F)F)c2c(=O)[nH]ncc21 ; CC(COCC(=O)N(C)C1CCN(c2ncc(C(F)(F)F)cn2)CC1)n1nc(C(F)(F)F)c2c(=O)[nH]ncc21 ; CC(COCC(=O)N(C)C1CCN(c2ncc(C(F)(F)F)cn2)CC1)n1cc(C(F)(F)F)c2c(=O)[nH]ncc21 ; CC(COCC(=O)N(C)C1CCN(c2ncc(C(F)(F)F)cn2)CC1)n1cc(C(F)(F)F)c2c(=O)[nH]ncc21]

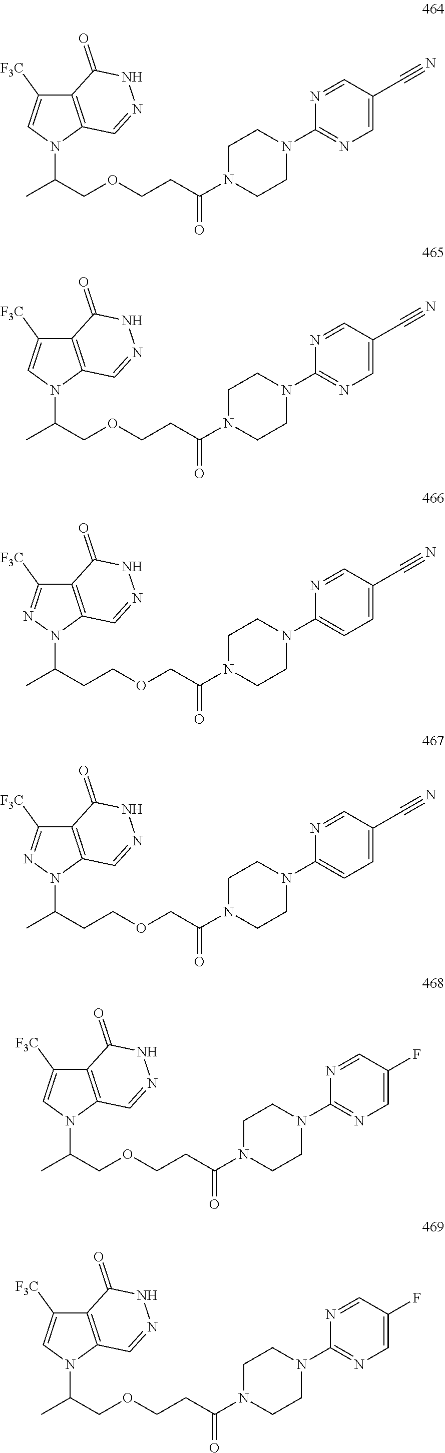 [SMILES: CC(COCCC(=O)N1CCN(c2ncc(C#N)cn2)CC1)n1cc(C(F)(F)F)c2c(=O)[nH]ncc21 ; CC(COCCC(=O)N1CCN(c2ncc(C#N)cn2)CC1)n1cc(C(F)(F)F)c2c(=O)[nH]ncc21 ; CC(CCOCC(=O)N1CCN(c2ccc(C#N)cn2)CC1)n1nc(C(F)(F)F)c2c(=O)[nH]ncc21 ; CC(CCOCC(=O)N1CCN(c2ccc(C#N)cn2)CC1)n1nc(C(F)(F)F)c2c(=O)[nH]ncc21 ; CC(COCCC(=O)N1CCN(c2ncc(F)cn2)CC1)n1cc(C(F)(F)F)c2c(=O)[nH]ncc21 ; CC(COCCC(=O)N1CCN(c2ncc(F)cn2)CC1)n1cc(C(F)(F)F)c2c(=O)[nH]ncc21]

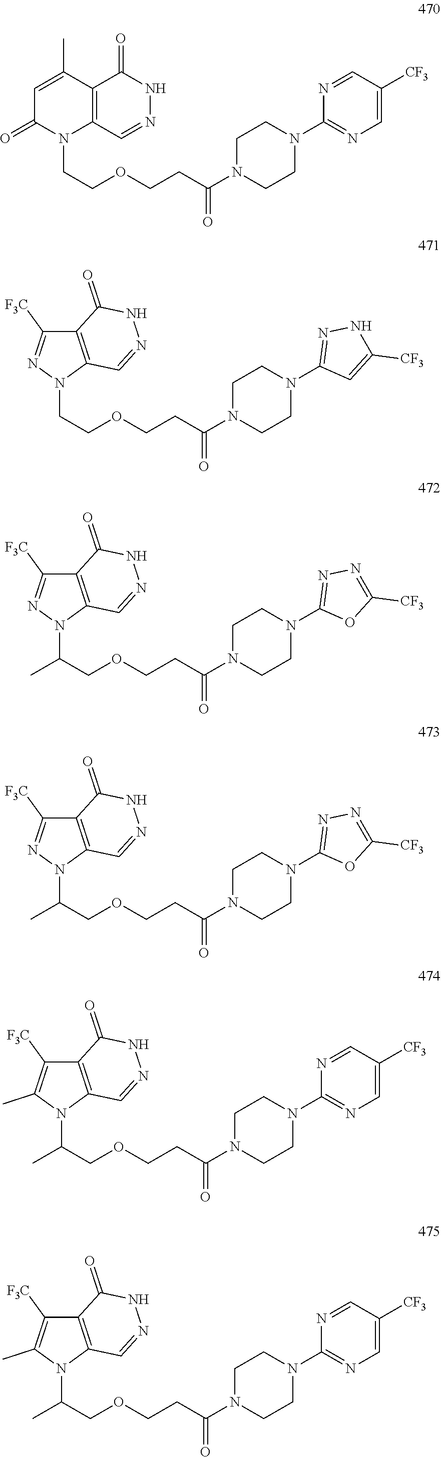 [SMILES: Cc1cc(=O)n(CCOCCC(=O)N2CCN(c3ncc(C(F)(F)F)cn3)CC2)c2cn[nH]c(=O)c12 ; O=C(CCOCCn1nc(C(F)(F)F)c2c(=O)[nH]ncc21)N1CCN(c2cc(C(F)(F)F)[nH]n2)CC1 ; CC(COCCC(=O)N1CCN(c2nnc(C(F)(F)F)o2)CC1)n1nc(C(F)(F)F)c2c(=O)[nH]ncc21 ; CC(COCCC(=O)N1CCN(c2nnc(C(F)(F)F)o2)CC1)n1nc(C(F)(F)F)c2c(=O)[nH]ncc21 ; Cc1c(C(F)(F)F)c2c(=O)[nH]ncc2n1C(C)COCCC(=O)N1CCN(c2ncc(C(F)(F)F)cn2)CC1 ; Cc1c(C(F)(F)F)c2c(=O)[nH]ncc2n1C(C)COCCC(=O)N1CCN(c2ncc(C(F)(F)F)cn2)CC1]

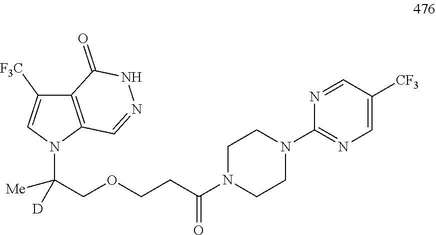 [SMILES: [2H]C(C)(COCCC(=O)N1CCN(c2ncc(C(F)(F)F)cn2)CC1)n1cc(C(F)(F)F)c2c(=O)[nH]ncc21]

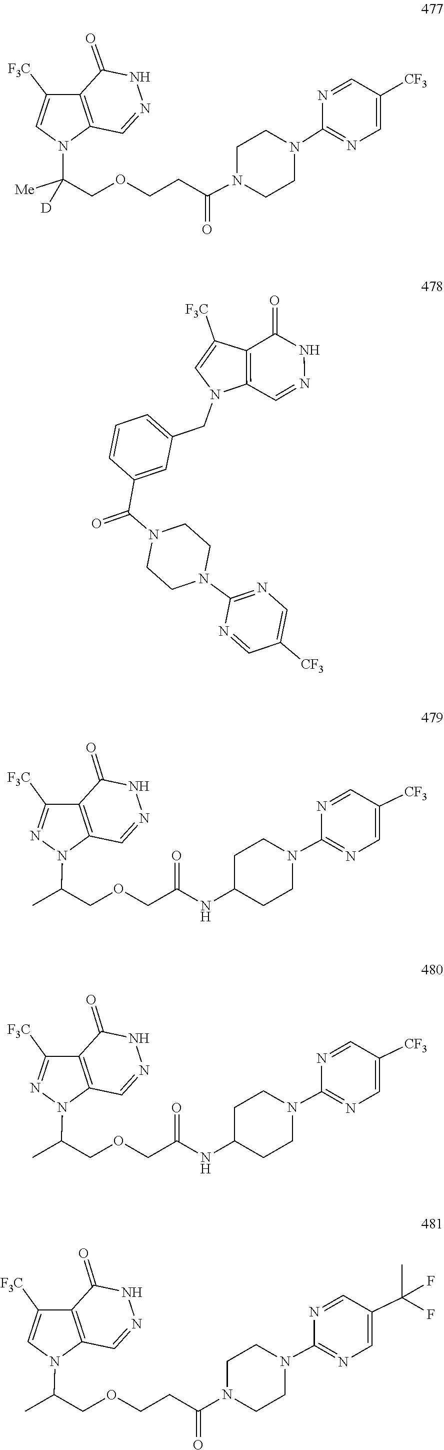 [SMILES: [2H]C(C)(COCCC(=O)N1CCN(c2ncc(C(F)(F)F)cn2)CC1)n1cc(C(F)(F)F)c2c(=O)[nH]ncc21 ; O=C(c1cccc(Cn2cc(C(F)(F)F)c3c(=O)[nH]ncc32)c1)N1CCN(c2ncc(C(F)(F)F)cn2)CC1 ; CC(COCC(=O)NC1CCN(c2ncc(C(F)(F)F)cn2)CC1)n1nc(C(F)(F)F)c2c(=O)[nH]ncc21 ; CC(COCC(=O)NC1CCN(c2ncc(C(F)(F)F)cn2)CC1)n1nc(C(F)(F)F)c2c(=O)[nH]ncc21 ; CC(COCCC(=O)N1CCN(c2ncc(C(C)(F)F)cn2)CC1)n1cc(C(F)(F)F)c2c(=O)[nH]ncc21]

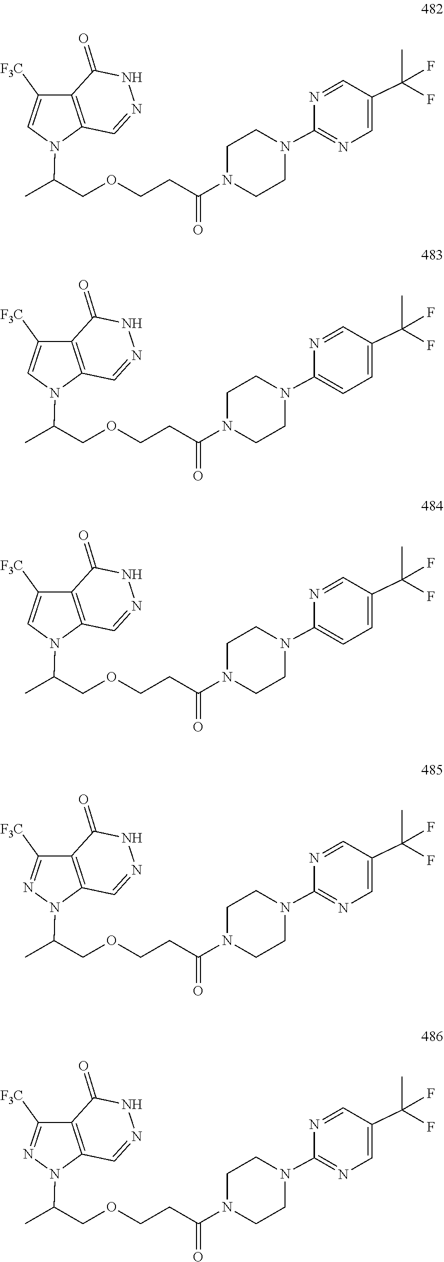 [SMILES: CC(COCCC(=O)N1CCN(c2ncc(C(C)(F)F)cn2)CC1)n1cc(C(F)(F)F)c2c(=O)[nH]ncc21 ; CC(COCCC(=O)N1CCN(c2ccc(C(C)(F)F)cn2)CC1)n1cc(C(F)(F)F)c2c(=O)[nH]ncc21 ; CC(COCCC(=O)N1CCN(c2ccc(C(C)(F)F)cn2)CC1)n1cc(C(F)(F)F)c2c(=O)[nH]ncc21 ; CC(COCCC(=O)N1CCN(c2ncc(C(C)(F)F)cn2)CC1)n1nc(C(F)(F)F)c2c(=O)[nH]ncc21 ; CC(COCCC(=O)N1CCN(c2ncc(C(C)(F)F)cn2)CC1)n1nc(C(F)(F)F)c2c(=O)[nH]ncc21]

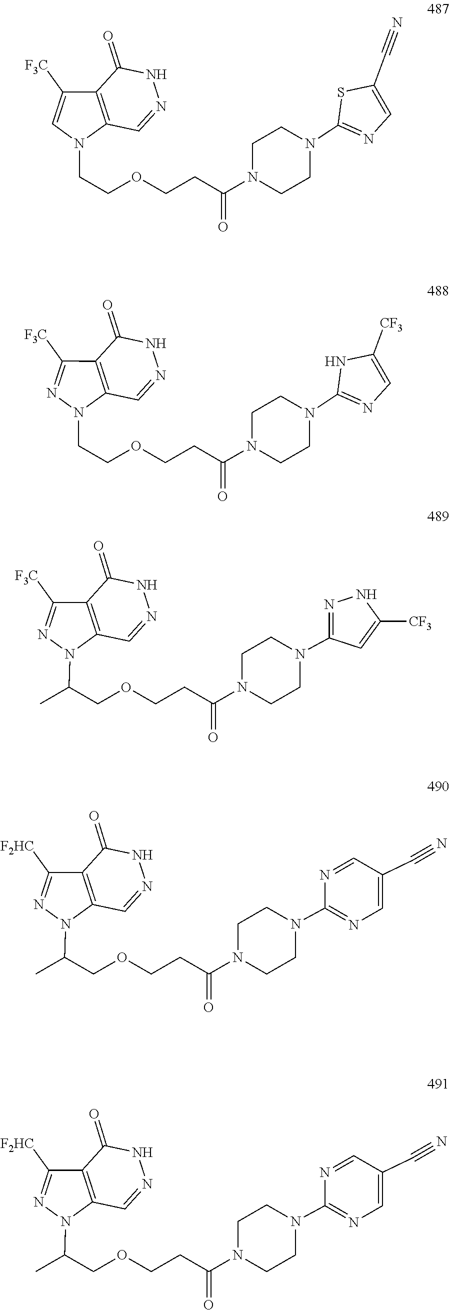 [SMILES: N#Cc1cnc(N2CCN(C(=O)CCOCCn3cc(C(F)(F)F)c4c(=O)[nH]ncc43)CC2)s1 ; O=C(CCOCCn1nc(C(F)(F)F)c2c(=O)[nH]ncc21)N1CCN(c2ncc(C(F)(F)F)[nH]2)CC1 ; CC(COCCC(=O)N1CCN(c2cc(C(F)(F)F)[nH]n2)CC1)n1nc(C(F)(F)F)c2c(=O)[nH]ncc21 ; CC(COCCC(=O)N1CCN(c2ncc(C#N)cn2)CC1)n1nc(C(F)F)c2c(=O)[nH]ncc21 ; CC(COCCC(=O)N1CCN(c2ncc(C#N)cn2)CC1)n1nc(C(F)F)c2c(=O)[nH]ncc21]

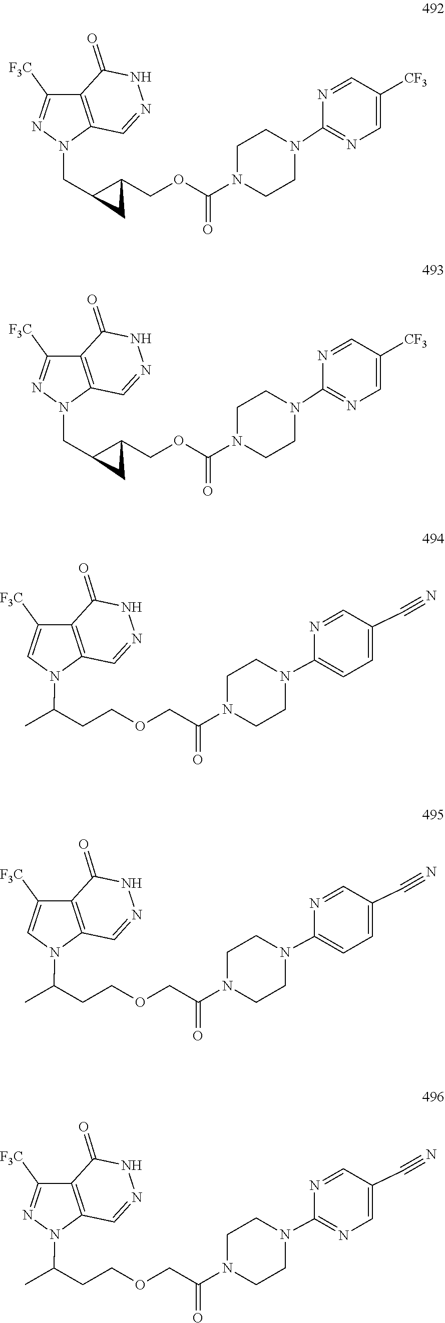 [SMILES: O=C(OC[C@@H]1C[C@H]1Cn1nc(C(F)(F)F)c2c(=O)[nH]ncc21)N1CCN(c2ncc(C(F)(F)F)cn2)CC1 ; O=C(OC[C@@H]1C[C@H]1Cn1nc(C(F)(F)F)c2c(=O)[nH]ncc21)N1CCN(c2ncc(C(F)(F)F)cn2)CC1 ; CC(CCOCC(=O)N1CCN(c2ccc(C#N)cn2)CC1)n1cc(C(F)(F)F)c2c(=O)[nH]ncc21 ; CC(CCOCC(=O)N1CCN(c2ccc(C#N)cn2)CC1)n1cc(C(F)(F)F)c2c(=O)[nH]ncc21 ; CC(CCOCC(=O)N1CCN(c2ncc(C#N)cn2)CC1)n1nc(C(F)(F)F)c2c(=O)[nH]ncc21]

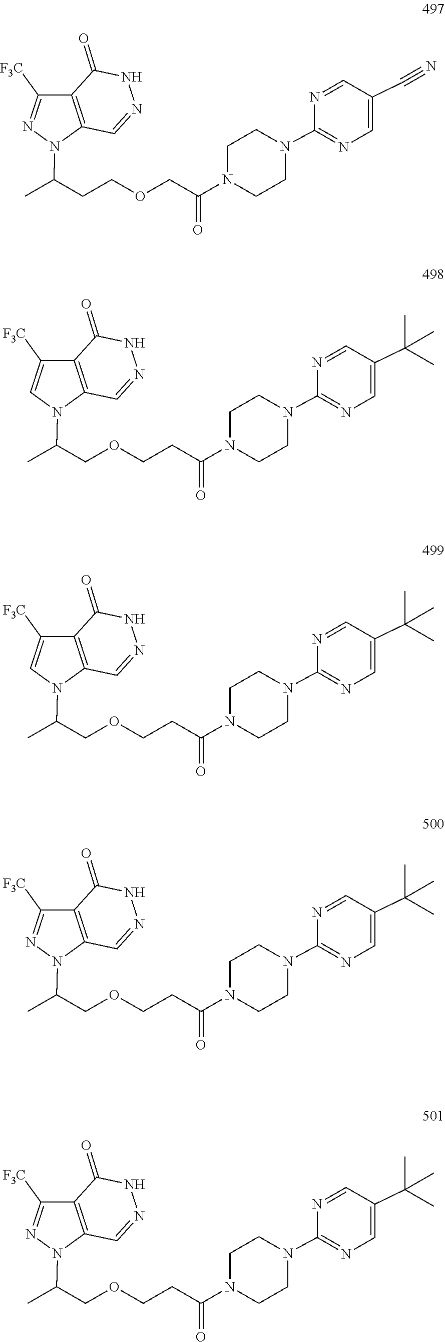 [SMILES: CC(CCOCC(=O)N1CCN(c2ncc(C#N)cn2)CC1)n1nc(C(F)(F)F)c2c(=O)[nH]ncc21 ; CC(COCCC(=O)N1CCN(c2ncc(C(C)(C)C)cn2)CC1)n1cc(C(F)(F)F)c2c(=O)[nH]ncc21 ; CC(COCCC(=O)N1CCN(c2ncc(C(C)(C)C)cn2)CC1)n1cc(C(F)(F)F)c2c(=O)[nH]ncc21 ; CC(COCCC(=O)N1CCN(c2ncc(C(C)(C)C)cn2)CC1)n1nc(C(F)(F)F)c2c(=O)[nH]ncc21 ; CC(COCCC(=O)N1CCN(c2ncc(C(C)(C)C)cn2)CC1)n1nc(C(F)(F)F)c2c(=O)[nH]ncc21]

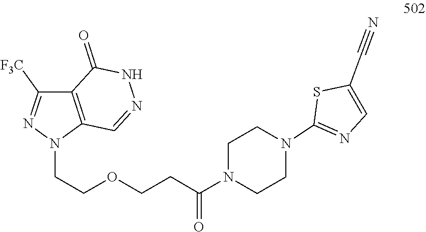 [SMILES: N#Cc1cnc(N2CCN(C(=O)CCOCCn3nc(C(F)(F)F)c4c(=O)[nH]ncc43)CC2)s1]

The compounds of the present invention have been described in detail above in terms of their structures. For the avoidance of doubt, any compounds for use in the invention may comprise compounds or compositions in accordance with their structure as follows:
an isolated enantiomer, or
a mixture of two or more enantiomers, or
a mixture of two or more diastereomers, and/or epimers, or
a racemic mixture, or
one or more tautomers;
   of each structure.

In the case of compound 116, this is the active enantiomer, eluted as a first fraction when a racemic mixture of the two enantiomers is applied to a Daicel CHIRALPAK chiral chromatography column.

In the case of compounds 33, 62, 63 and 273, these are each the active enantiomer, eluted as a second fraction when a racemic mixture of the two enantiomers is applied to a Daicel CHIRALPAK chiral chromatography column.

In the case of the following pairs of compounds: 64 and 65, 70 and 71, 72 and 73, 75 and 76, 85 and 86, 87 and 88, 89 and 90, 119 and 120, 122 and 123, 129 and 130, 137 and 138, 142 and 143, 148 and 149, 151 and 152, 155 and 156, 160 and 161, 162 and 163, 168 and 169, 174 and 175, 176 and 177, 178 and 179, 185 and 186, 187 and 188, 189 and 190,201 and 202, 209 and 210, 211 and 212, 221 and 222, 223 and 224, 243 and 244, 245 and 246, 248 and 249, 250 and 251, 253 and 254, 255 and 256, 258 and 259, 262 and 263, 264 and 265, 266 and 267, 274 and 275, 278 and 279, 280 and 281, 283 and 284, 286 and 287, 288 and 289, 291 and 292, 293 and 294, 297 and 298, 304 and 305, 306 and 307, 308 and 309, 310 and 311, 312 and 313, 318 and 319, 321 and 322, 323 and 324, 325 and 326, 327 and 328, 329 and 330, 331 and 332, 335 and 336, 338 and 339, 342 and 343, 344 and 345, 347 and 348, 349 and 350, 351 and 352, 353 and 354, 355 and 356, 365 and 366, 368 and 389, 370 and 371, 372 and 373, 374 and 375, 376 and 377, 386 and 387, 388 and 389, 392 and 393, 395 and 396, 397 and 398, 399 and 400, 405 and 406,411 and 412, 414 and 415, 416 and 417, 418 and 419, 420 and 421, 422 and 423, 426 and 427, 429 and 430, 431 and 432, 435 and 436, 437 and 438, 439 and 440, 442 and 443, 444 and 445, 448 and 449, 450 and 451, 452 and 453, 454 and 455, 456 and 457, 458 and 459, 460 and 461, 462 and 463, 464 and 465, 466 and 467, 468 and 469, 472 and 473, 474 and 475, 476 and 477, 479 and 480, 481 and 482, 483 and 484, 485 and 486, 490 and 491, 492 and 493, 494 and 495, 496 and 497, 498 and 499, and 500 and 501; these are each a pair of enantiomers, eluted as first and second fractions respectively, when a racemic mixture of the two enantiomers is applied to a Daicel CHIRALPAK chiral chromatography column.

The compounds described herein may be provided for use in medicine. In the context of the present invention, the medicinal use is not especially limited, provided that it is a use which is facilitated by the PARP7 inhibitory effect of the compound. Thus, the compounds of the invention may be for use in any disease, condition or disorder that may be prevented, ameliorated or treated using a PARP7 inhibitor. Typically, this comprises a disease condition and/or a disorder selected from: a cancer, an infectious disease, a central nervous system disease or disorder, and a pain condition.

When the disease, condition or disorder is a cancer, it is not especially limited, provided that the cancer is one which may be treated, prevented or ameliorated by using a PARP7 inhibitor. Thus the cancer may be a cancer selected from: a solid or liquid tumour including cancer of the eye, brain (such as gliomas, glioblastomas, medullablastomas, craniopharyngioma, ependymoma, and astrocytoma), spinal cord, kidney, mouth, lip, throat, oral cavity, nasal cavity, small intestine, colon, parathyroid gland, gall bladder, head and neck, breast, bone, bile duct, cervix, heart, hypopharyngeal gland, lung, bronchus, liver, skin, ureter, urethra, testicles, vagina, anus, laryngeal gland, ovary, thyroid, oesophagus, nasopharyngeal gland, pituitary gland, salivary gland, prostate, pancreas, adrenal glands; an endometrial cancer, oral cancer, melanoma, neuroblastoma, gastric cancer, an angiomatosis, a hemangioblastoma, a pheochromocytoma, a pancreatic cyst, a renal cell carcinoma, Wilms' tumour, squamous cell carcinoma, sarcoma, osteosarcoma, Kaposi sarcoma, rhabdomyosarcoma, hepatocellular carcinoma, PTEN Hamartoma-Tumor Syndromes (PHTS) (such as Lhermitte-Duclos disease, Cowden syndrome, Proteus syndrome, and Proteus-like syndrome), leukaemias and lymphomas (such as acute lymphoblastic leukaemia, chronic lymphocytic leukaemia, acute myelogenous leukaemia, chronic myelogenous leukaemia, hairy cell leukaemia, T-cell prolymphocytic leukemia (T-PLL), large granular lymphocytic leukemia, adult T-cell leukemia, juvenile myelomonocytic leukaemia, Hodgkin lymphoma, non-Hodgkin lymphoma, mantle lymphoma, follicular lymphoma, primary effusion lymphoma, AIDS-related lymphoma, Hodgkin lymphoma, diffuse B cell lymphoma, Burkitt lymphoma, and cutaneous T-cell lymphoma), preferably wherein the cancer is a cancer selected from oesaphageal, head and neck, non-small cell lung cancer, squamous cell cancer of the lung, breast, acute myeloid leukemia (AML), a small-cell lung cancer, a melanoma, an ovarian cancer, a colorectal cancer, a pancreatic cancer, an endometrial cancer, and a skin papilloma.

When the disease is an infectious disease, it is not especially limited, provided that the disease is one which may be treated, prevented or ameliorated by using a PARP7 inhibitor. However, typically the infectious disease is selected from a bacterial infection and a viral infection, preferably a respiratory infection, immune system infection, gut infection and sepsis. Such viral respiratory infections include influenza and coronavirus infections, particularly influenza A and SARS-CoV-2 infections.

When the disease, condition or disorder is a central nervous system disease, condition or disorder, it is not especially limited, provided that the disease, condition or disorder is one which may be treated, prevented or ameliorated by using a PARP7 inhibitor. However, the central nervous system disease, condition or disorder is typically selected from amyotrophic lateral sclerosis (AML), Huntington's disease, Alzheimer's disease, pain, a psychiatric disorder, multiple sclerosis, Parkinson's disease, and HIV related neurocognitive decline.

When the disease, condition or disorder is a pain condition it is not especially limited, provided that the condition is one which may be treated, prevented or ameliorated by using a PARP7 inhibitor. Typically, the pain condition is nociceptive pain or neuropathic pain and may be a chronic pain condition such as cancer-associated pain and peripheral neuropathy.

The present invention also provides a pharmaceutical composition comprising a compound as defined above. Whilst the pharmaceutical composition is not especially limited, typically the composition further comprises a pharmaceutically acceptable additive and/or excipient. In the pharmaceutical composition, the compound as defined above may be present in the form described above, but may alternatively be in a form suitable for improving bioavailability, solubility, and/or activity, and/or may be in a form suitable for improving formulation. Thus, the compound may be in the form of a pharmaceutically acceptable salt, hydrate, acid, ester, or other alternative suitable form. Typically, the composition is for treating a disease, condition or disorder as defined above. In some instances, the compound may be present in the composition as a pharmaceutically acceptable salt, or other alternative form of the compound, in order to ameliorate pharmaceutical formulation.

In some embodiments the pharmaceutical composition is a composition for treating a cancer, further comprising a further agent for treating cancer. The further agent for treating cancer is not especially limited, provided that it affords some utility for cancer treatment. However, typically the further agent for treating cancer is selected from anti-microtubule agents, platinum coordination complexes, alkylating agents, antibiotic agents, topoisomerase II inhibitors, antimetabolites, topoisomerase I inhibitors, senolytic agents, hormones and hormone analogues, signal transduction pathway inhibitors, DNA damage repair pathway inhibitors, non-receptor tyrosine kinase angiogenesis inhibitors, immunotherapeutic agents (such as an anti-tumour vaccine, an oncolytic virus, an immune stimulatory antibody such as anti-CTLA4, anti-PD1, anti-PDL-1, anti-OX40, anti-41BB, anti-CD27, anti-CD40, anti-LAG3, anti-TIM3, and anti-GITR, a novel adjuvant, a peptide, a cytokine, a chimeric antigen receptor T cell therapy (CAR-T), a small molecule immune modulator such as an IDO or TDO inhibitor or a pattern recognition receptor agonist such as a STING, TLR-9 or RIG-I Helicase agonist, tumour microenvironment modulators, and anti-angiogenic agents), receptor tyrosine kinase inhibitors, cell growth inhibitors such as Ras and Raf inhibitors, proapoptotic agents and cell cycle signalling inhibitors.

In still further embodiments the invention provides a pharmaceutical kit for treating a cancer, which pharmaceutical kit comprises:
(a) a compound as defined above; and
(b) a further agent for treating cancer; preferably wherein the further agent for treating cancer is selected from anti-microtubule agents, platinum coordination complexes, alkylating agents, antibiotic agents, topoisomerase II inhibitors, antimetabolites, topoisomerase I inhibitors, senolytic agents, hormones and hormone analogues, signal transduction pathway inhibitors, DNA damage repair pathway inhibitors, non-receptor tyrosine kinase angiogenesis inhibitors, immunotherapeutic agents (such as an anti-tumour vaccine, an oncolytic virus, an immune stimulatory antibody such as anti-CTLA4, anti-PD1, anti-PDL-1, anti-OX40, anti-41BB, anti-CD27, anti-CD40, anti-LAG3, anti-TIM3, and anti-GITR, a novel adjuvant, a peptide, a cytokine, a chimeric antigen receptor T cell therapy (CAR-T), a small molecule immune modulator such as a pattern recognition receptor agonist such as a STING, TLR-9 or RIG-I Helicase agonist, tumour microenvironment modulators, and anti-angiogenic agents), receptor tyrosine kinase inhibitors, cell growth inhibitors such as Ras and Raf inhibitors, proapoptotic agents and cell cycle signalling inhibitors;
   wherein the compound and the further agent are suitable for administration simultaneously, sequentially or separately.

Further provided by the invention is a method of treating a disease and/or a condition and/or a disorder, which method comprises administering to a patient (or subject) a compound, or a composition, or a kit as defined above. The method is typically a method for treating any disease condition or disorder mentioned herein. In typical embodiments, the method is a method for treating a cancer. Preferably such a method comprises administering to a patient (or subject) a compound or a composition as defined above and a further agent for treating cancer as defined above. The compound or composition and the further agent may administered simultaneously, sequentially or separately, depending upon the agents and patients involved, and the type of cancer indicated.

Typically, in all embodiments of the invention, both above and below, the patient (or subject) is an animal, typically a mammal, including canines and felines, and more typically a human.

Further provided by the invention is a method of synthesis of a compound as defined above, which method comprises conducting a reaction between (i) a first reactant comprising rings A and B bearing a portion of substituent group R¹ and (ii) a second reactant comprising the remainder of substituent group R¹ so as to form the PARP7 inhibitor compound.

In one typical method the first reactant comprises a compound of general formula:

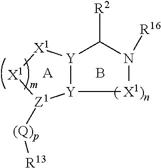

and the second reactant comprises a compound of general formula:

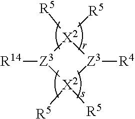

wherein R¹³ and R¹⁴ are each independently substituent groups which are removed during the reaction; and wherein X¹, Y, Z¹, Z², R², R⁴, R⁵, Q, m, n and p are as defined herein.

In typical embodiments, this method of synthesis is carried out by reacting under conditions suitable for an amide formation, nucleophilic displacement or Michael addition reaction. The skilled person may select the reaction conditions, with reference to known synthesis techniques depending on the appropriate starting materials. In some embodiments, the method comprises one or more additional substitution steps. Exemplary syntheses are shown in the Examples herein Typically, the above formulae (and all formulae herein) are shown in non-stereoisomeric form. For the avoidance of doubt, throughout the present disclosure a single formula is intended to represent all possible stereoisomers of a particular structure, including all possible isolated enantiomers corresponding to the formula, all possible mixtures of enantiomers corresponding to the formula, all possible mixtures of diastereomers corresponding to the formula, all possible mixtures of epimers corresponding to the formula and all possible racemic mixtures corresponding to the formula. In addition to this, the above formulae (and all formulae herein) are intended to represent all tautomeric forms equivalent to the corresponding formula.

There is further disclosed a PARP7 inhibitor compound, which compound comprises the following formula:

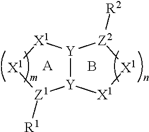

wherein each X¹ may be the same or different and is independently selected from C, N, O and S; each Y may be the same or different and is independently selected from C and N; Z¹ and Z² may be the same or different; Z¹ is independently selected from C and N; Z² is independently selected from C, N, O and S; each X¹ may independently be unsubstituted, or may independently be substituted with H or a substituted or unsubstituted organic group; each Y may independently be unsubstituted, or may independently be substituted with H or a substituted or unsubstituted organic group; Z¹ may independently be further substituted with H or a substituted or unsubstituted organic group; Z² may independently be further substituted with H or a substituted or unsubstituted organic group; m may be 1, 2, 3 or 4; n may be 1, 2, 3, or 4; the bonds between all of the atoms in ring A may independently be single bonds or double bonds provided that when X¹ is 0 or S the bonds to that X¹ are single bonds; the bonds between all of the atoms in ring B may independently be single bonds or double bonds provided that when X¹ or Z² is O or S the bonds to that X¹ or Z² are single bonds; and wherein R¹ may be attached to Z¹ by a single bond or a double bond and is a substituent of formula:

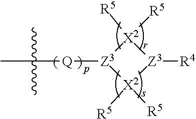

wherein each Q may be the same or different and is independently selected from C, N, O and S; each Q may independently be attached to another Q, or to Z³, by a single bond or a double bond; each Q may independently be unsubstituted, or may independently be substituted by H or a substituted or unsubstituted organic group; two or more Q atoms may form a ring together with their substituents; p is a number from 2 to 8; each Z³ may be the same or different and is independently selected from C and N; each Z³ may independently be further substituted with H or a substituted or unsubstituted organic group; each X² may be the same or different and is independently selected from C, N, O and S, preferably from C and N; r is a number from 1 to 5; s is independently a number from 1 to 5; R⁴ is a substituted or unsubstituted organic group comprising a substituted or unsubstituted carbocyclic or heterocyclic ring; each bond in the ring comprised of Z³ and X² atoms may independently be a double bond or a single bond, provided that when X² is O or S the bonds to that X² are single bonds; each R⁵ may be present or absent depending on the number of bonds to, and the valence of, the X² atom attached to that R⁵; and wherein each R⁵ is independently selected from H or a substituted or unsubstituted organic group;
and wherein R² may be present or absent, and when present may be attached to Z² by a single bond or a double bond and is selected from H or a substituted or unsubstituted organic group.

Typically, when Z² is C, R² is present and is selected from a substituted or unsubstituted organic group.

There is further disclosed a PARP7 inhibitor compound, which compound comprises the following formula:

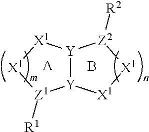

wherein each X¹ may be the same or different and is independently selected from C, N, O and S; each Y may be the same or different and is independently selected from C and N; Z¹ and Z² may be the same or different; Z¹ is independently selected from C and N; Z² is independently selected from C, N, O and S; each X¹ may independently be unsubstituted, or may independently be substituted with H or a substituted or unsubstituted organic group; each Y may independently be unsubstituted, or may independently be substituted with H or a substituted or unsubstituted organic group; Z¹ may independently be further substituted with H or a substituted or unsubstituted organic group; Z² may independently be further substituted with H or a substituted or unsubstituted organic group; m may be 1, 2, or 3; n may be 1, 2, or 3; the bonds between all of the atoms in ring A may independently be single bonds or double bonds provided that when X¹ is O or S the bonds to that X¹ are single bonds; the bonds between all of the atoms in ring B may independently be single bonds or double bonds provided that when X¹ or Z² is O or S the bonds to that X¹ or Z² are single bonds;
and wherein R¹ is as defined herein.

The term “comprises” as used throughout the description and claims herein means “includes or consists of”. The term denotes the inclusion of at least the features following the term and does not exclude the inclusion of other features which have not been explicitly mentioned. The term may also denote an entity which consists only of the features following the term.

### DETAILED DESCRIPTION OF THE INVENTION

The invention will now be described in more detail, by way of example only, with reference to the following specific embodiments and the accompanying drawings, in which:

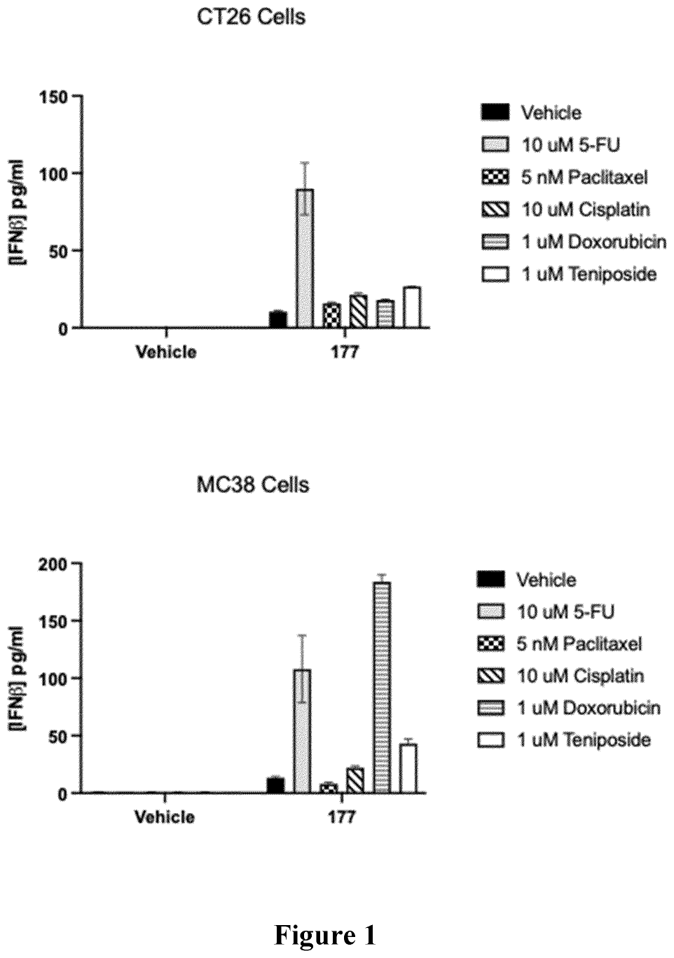

FIG. 1 shows induction of type I interferon production in CT26 and MC38 cancer cells in the presence of a compound according to the invention.

### EXAMPLES

### Exemplary Syntheses of Compounds of the Invention

The compounds of the invention may be synthesised using readily available starting materials and known reactions.

Exemplary syntheses of various compounds are shown below:

### 1 Synthesis of 6-(4-(3-((1-oxo-1,2-dihydrophthalazin-5-yl)methyl)benzoyl)piperazin-1-yl)nicotinonitrile (Compound 1)

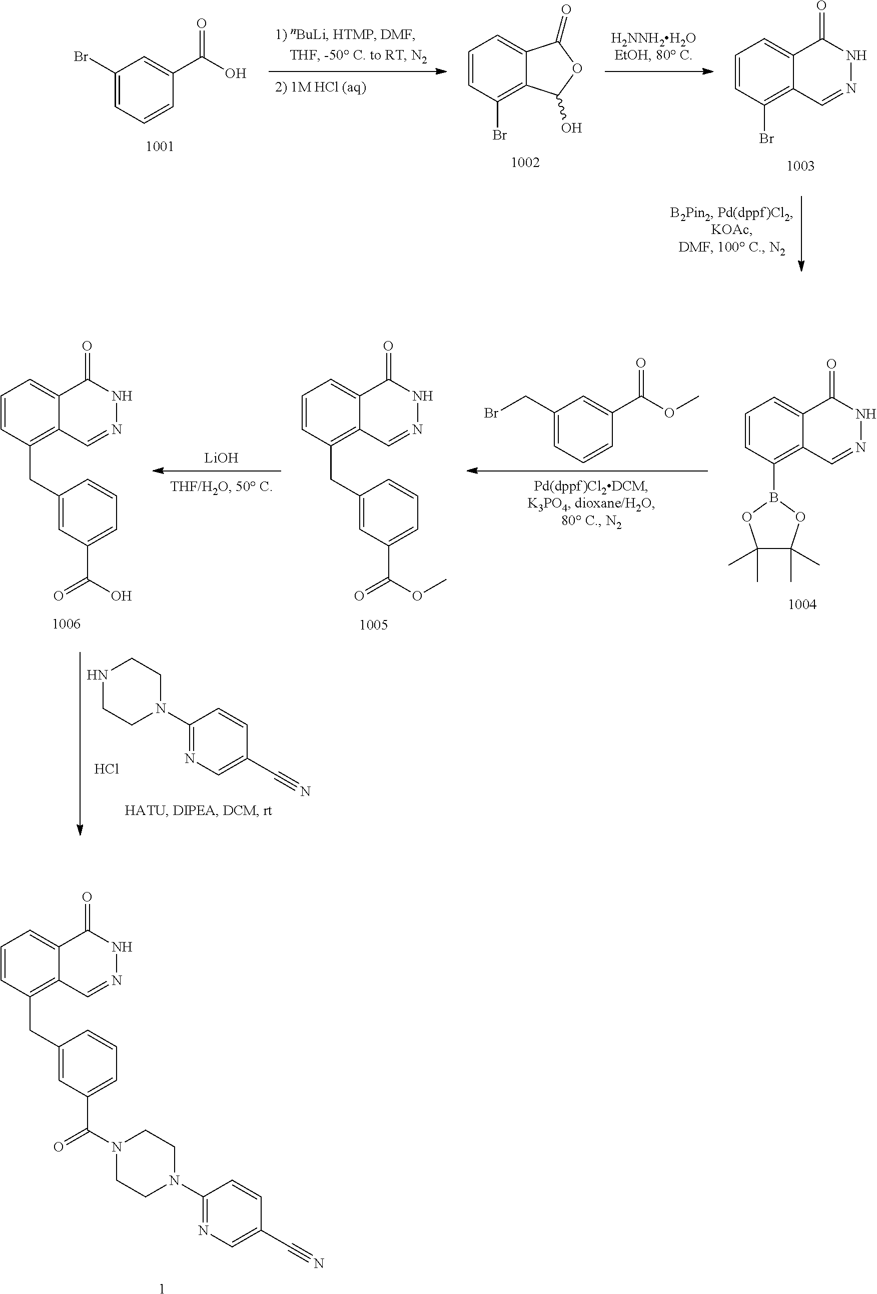

### Preparation of 4-bromo-3-hydroxyisobenzofuran-1(3H)-one (1002)

To a stirred solution of 2,2,6,6-tetramethylpiperidine (1.9 mL, 11.5 mmol) in anhydrous THE (10 mL) was added nBuLi (2.4 M in hexanes, 4.8 mL, 11.5 mmol) dropwise at −20° C. under N₂. After cooling to −50° C., 3-bromobenzoic acid 1001 (1 g, 5 mmol) in THE (10 mL) was added dropwise and the reaction mixture was stirred for 2 h at −50° C. Then N, N-dimethylformamide (1.9 mL, 25.0 mmol) was added dropwise at this temperature. The resulting reaction mixture was then warmed to room temperature slowly and stirred for additional 14 h. The reaction mixture was poured into ice-water (20 mL) carefully and stirred for 10 min. Then the mixture was washed with EtOAc (20 mL) twice. The obtained aqueous layer was acidified with 1 M HCl aqueous at 0° C. and extracted with EtOAc (30 mL×3). The organic layers were combined, dried over Na₂SO₄, filtered and concentrated in vacuo. The residue was purified by flash chromatography (eluting with petroleum ether/EtOAc=80:20 to 0:100). After concentration, the resulting solid was triturated with PE (x mL) to afford 4-bromo-3-hydroxyisobenzofuran-1(3H)-one 1002 (0.2 g, 95% purity, 16% yield) as a light yellow solid.

LCMS (ESI) calcd for C₈H₅BrO₃ [M−H]⁻ m/z 226.93, found 227.

### Preparation of 5-bromophthalazin-1(2H)-one (1003)

To a solution of 4-bromo-3-hydroxyisobenzofuran-1(3H)-one 1002 (100 mg, 0.44 mmol) in ethanol (5 mL) was added hydrazine hydrate (80%, 136.3 mg, 2.18 mmol) in one portion. The reaction mixture was stirred at 80° C. for 2 h. The reaction mixture was cooled to rt and then filtered. The filter cake was rinsed with EtOH (5 mL×2) and then dried in vacuo to give 5-bromophthalazin-1(2H)-one 1003 (90 mg, 95% purity, 87% yield) as white needles.

LCMS (ESI) calcd for C₈H₅BrN₂O [M+H]⁺ m/z 224.97, found 225.

### Preparation of 5-(4,4,5,5-tetramethyl-1,3,2-dioxaborolan-2-yl)phthalazin-1(2H)-one (1004)

In a three-neck flask, equipped with stirring bar, condenser and a rubber septum, thoroughly purged with N₂, were introduced 5-bromophthalazin-1(2H)-one 1003 (2 g, 8.9 mmol), Pd(dppf)Cl₂ (0.65 g, 0.8 mmol), B₂Pin₂ (5.65 g, 22.2 mmol) and potassium acetate (2.62 g, 26.7 mmol). The flask was purged with N₂ once more before adding DMF (100 mL) via syringe. The resulting mixture was stirred at 100° C. for 1 h. After cooling to room temperature, the reaction mixture was poured into cold water and then extracted with EtOAc (200 mL×4). The combined organic layer was concentrated under reduced pressure. The residue was purified by flash chromatography (eluting with petroleum ether/EtOAc=20:80 to 0:100) to afford 5-(4,4,5,5-tetramethyl-1,3,2-dioxaborolan-2-yl)phthalazin-1(2H)-one 1004 (0.5 g, 95% purity, 19% yield) as a white solid.

LCMS (ESI) calcd for C₁₄H₁₇BN₂O₃[M+H]⁺ m/z 272.14, found 273.

### Preparation of methyl 3-((1-oxo-1,2-dihydrophthalazin-5-yl)methyl)benzoate (1005)

To a stirred solution of 5-(4,4,5,5-tetramethyl-1,3,2-dioxaborolan-2-yl)phthalazin-1(2H)-one 1004 (500 mg, 1.84 mmol) and methyl 3-(bromomethyl)benzoate (463 mg, 2.02 mmol) in 1,4-dioxane/H₂O (50 mL, 4:1) was added K₃PO₄ (1170 mg, 5.51 mmol) at room temperature. Nitrogen was purged into the reaction mixture for 5 min before adding Pd(pddf)Cl₂·DCM (150 mg, 0.18 mmol, the mixture was subsequently purged with N₂ for additional 5 min. The reaction mixture was stirred at 80° C. for 1.5 h. The reaction mixture was cooled to rt, diluted with water (30 mL) and extracted with EtOAc (50 mL×3). The combined organic layer was washed with brine, dried over Na₂SO₄ and concentrated in vacuo. The residue was purified by flash chromatography (eluting with PE/EtOAc=60:40˜0:100) to give methyl 3-((1-oxo-1,2-dihydrophthalazin-5-yl)methyl)benzoate 1005 (500 mg, 85% purity, 78% yield) as a brown solid.

LCMS (ESI) calcd for C₁₇H₁₄N₂O₃ [M+H]⁺ m/z 295.11, found 295.

### Preparation of 3-((1-oxo-1,2-dihydrophthalazin-5-yl)methyl)benzoic acid (1006)

To a solution of methyl 3-((1-oxo-1,2-dihydrophthalazin-5-yl)methyl)benzoate 1005 (500 mg, 1.70 mmol) in THF/H₂O (40 mL, 3:1) was added LiOH (203 mg, 8.49 mmol). The mixture was stirred at 50° C. for 1 h. THE was removed under reduced pressure and the aqueous phase was acidified with 1 M HCl aq. to pH=4˜5. The resulting solid was collected by filtration and dried in vacuo to obtain 3-((1-oxo-1,2-dihydrophthalazin-5-yl)methyl)benzoic acid 1006 (350 mg, 85% purity, 62% yield) as a gray solid.

LCMS (ESI) calcd for C₁₆H₁₂N₂O₃ [M+H]⁺ m/z 281.09, found 281.

### Preparation of 6-(4-(3-((1-oxo-1,2-dihydrophthalazin-5-yl)methyl)benzoyl)piperazin-1-yl)nicotinonitrile (1)

To a solution of 3-((1-oxo-1,2-dihydrophthalazin-5-yl)methyl)benzoic acid 1006 (150 mg, 0.54 mmol) in DCM (15 mL) was added HATU (305 mg, 0.80 mmol), DIPEA (208 mg, 1.61 mmol) and 6-(piperazin-1-yl)pyridine-3-carbonitrile hydrochloride (132 mg, 0.59 mmol) at room temperature successively. The mixture was kept stirring at room temperature for 1 h. The resulting mixture was diluted with water and extracted with DCM (30 mL×3). The combined organic layer was dried over Na₂SO₄ and concentrated under reduced pressure. The residue was purified by pre-HPLC (columns: Shim-pack GIST 5 um C18 20×250 mm, mobile phase: ACN-H₂O (0.1% FA), gradient: 35-75) to give 6-(4-(3-((1-oxo-1,2-dihydrophthalazin-5-yl)methyl)benzoyl)piperazin-1-yl)nicotinonitrile 1 (35.7 mg, 99% purity, 14% yield) as a white solid.

¹H NMR (400 MHz, DMSO-d₆, ppm) δ: 12.72 (s, 1H), 8.56 (s, 1H), 8.51 (d, J=2.4 Hz, 1H), 8.15 (dd, J=7.0, 1.8 Hz, 1H), 7.90 (dd, J=9.0, 2.2 Hz, 1H), 7.85-7.77 (m, 2H), 7.42-7.25 (m, 4H), 6.92 (d, J=9.2 Hz, 1H), 4.49 (s, 2H), 3.83-3.53 (m, 6H), 3.38 (s, 2H).

LCMS (ESI) calcd for C₂₆H₂₂N₆O₂ [M+H]⁺ m/z 451.19, found 451.

### 2 Synthesis of 1-(2-(3-oxo-3-(4-(5-(trifluoromethyl)pyrimidin-2-yl)piperazin-1-yl)propoxy)ethyl)-1,5-dihydro-4H-pyrrolo[2,3-d]pyridazin-4-one (Compound 10)

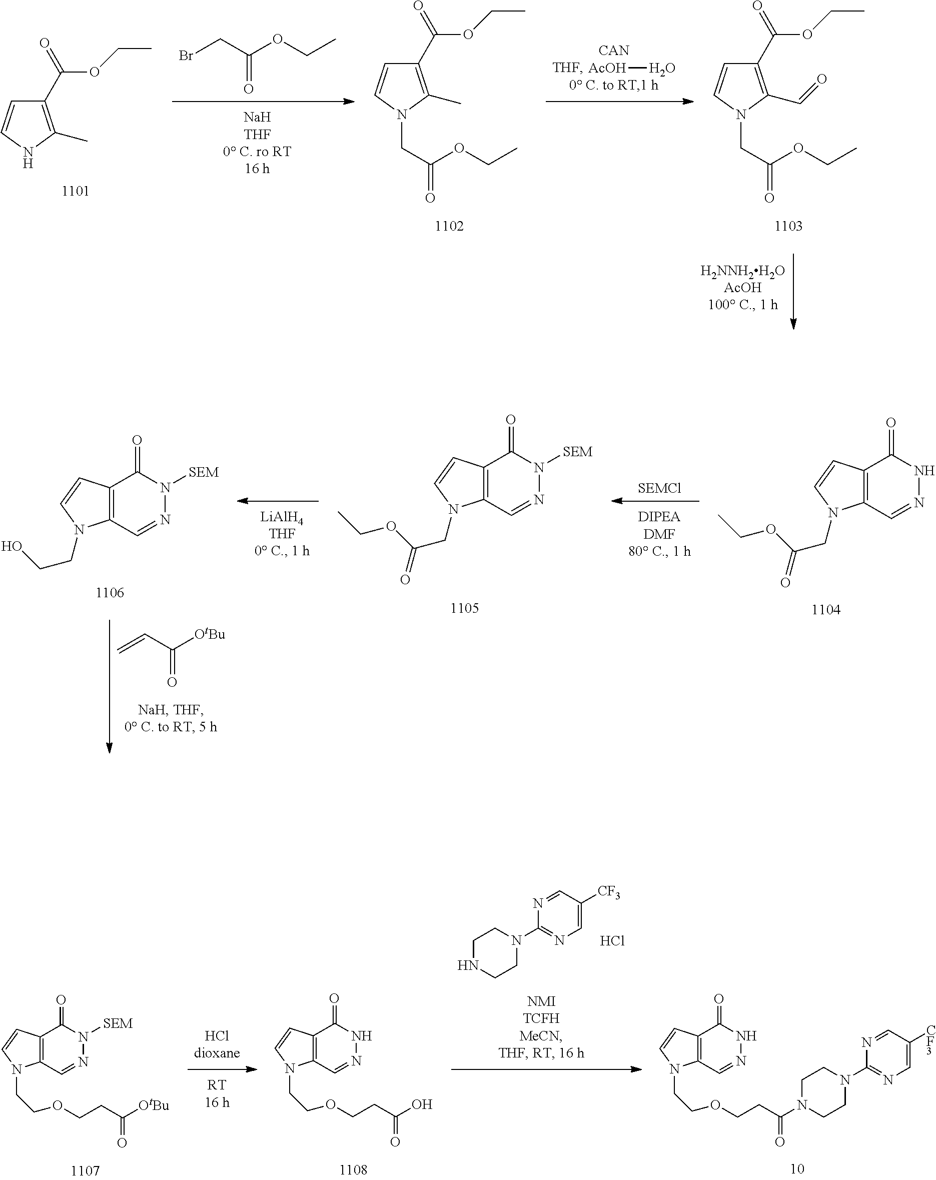

### Preparation of ethyl 1-(2-ethoxy-2-oxoethyl)-2-methyl-1H-pyrrole-3-carboxylate (1102)

To a stirred solution of NaH (60% wt, 1.56 g, 39.1 mmol) in THE (100 mL) at 0° C. was added ethyl 2-methyl-1H-pyrrole-3-carboxylate 1101 (5 g, 32.6 mmol) in portions. After stirring 15 min at 0° C., ethyl 2-bromoacetate (6 g, 35.9 mmol) was added dropwise and the reaction was warmed to rt and stirred for 16 h. The reaction was quenched with saturated aqueous NH₄Cl and extracted with EtOAc (50 mL×4). The combined organic layers were washed with brine (30 mL), dried over Na₂SO₄ and concentrated in vacuo. The residue was purified by flash chromatography (eluting with petroleum ether/EtOAc=90 to 60:40) to afford ethyl 1-(2-ethoxy-2-oxoethyl)-2-methyl-1H-pyrrole-3-carboxylate 1102 (7 g, 90% purity, 80% yield) as an off-white solid.

LCMS (ESI) calcd for C₁₂H₁₇NO₄ [M+H]⁺ m/z 240.12, found 240.

### Preparation of ethyl 1-(2-ethoxy-2-oxoethyl)-2-formyl-1H-pyrrole-3-carboxylate (1103)

Ethyl 1-(2-ethoxy-2-oxoethyl)-2-methyl-1H-pyrrole-3-carboxylate 1102 (7 g, 29.3 mmol) was dissolved in THE (120 mL) under stirring, followed by addition a solution of AcOH (140 mL) and H₂O (120 mL). The mixture was homogeneously stirred at 0° C. and cerium(IV) ammonium nitrate (64 g, 117.2 mol) was added in one portion. After stirring at rt for 1 h, the reaction mixture was poured into ice-water (300 mL) and stirred for another 30 min. The resulting solution was extracted with EtOAc (200 mL×3). The combined organic layer was washed with brine, dried over Na₂SO₄ and concentrated under reduced pressure. The residue was purified by flash chromatography (eluting with petroleum ether/EtOAc=100:0 to 80:20) to obtain the title compound ethyl 1-(2-ethoxy-2-oxoethyl)-2-formyl-1H-pyrrole-3-carboxylate 1103 (3 g, 95% purity, 38% yield) as a yellow solid.

LCMS (ESI) calcd for C₁₂H₁₅NO₅ [M+H]⁺ m/z 254.10, found 254.

### Preparation of ethyl 2-(4-oxo-4,5-dihydro-1H-pyrrolo[2,3-d]-pyridazin-1-yl)-acetate (1104)

To a solution of ethyl 1-(2-ethoxy-2-oxoethyl)-2-formyl-1H-pyrrole-3-carboxylate 1103 (3 g, 11.8 mmol) in AcOH (30 mL) was added H₂NNH₂·H₂O (80% wt, 1.11 g, 17.7 mmol) in one portion. The reaction mixture was heated with stirring at 100° C. for 1 h. Most of the solvent was removed by evaporation (55° C.) under reduced pressure. The residue was cooled in an ice-water bath. The precipitate formed was collected and then rinsed with water (10 mL×2). The solid was dried (55° C.) in vacuo to give ethyl 2-(4-oxo-4,5-dihydro-1H-pyrrolo[2,3-d]-pyridazin-1-yl)-acetate 1104 (1.9 g, 95% purity, 69% yield) as a yellow solid.

LCMS (ESI) calcd for C₁₀H₁₁N₃O₃[M+H]⁺ m/z 222.09, found 222.

### Preparation of ethyl 2-(4-oxo-5-((2-(trimethylsilyl)ethoxy)methyl)-4,5-dihydro-1H-pyrrolo[2,3-d]pyridazin-1-yl)acetate (1105)

To a solution of ethyl 2-(4-oxo-4,5-dihydro-1H-pyrrolo[2,3-d]-pyridazin-1-yl)-acetate 1104 (1.45 g, 6.6 mmol) and DIPEA (4.26 g, 33.0 mmol) in DMF (20 mL) at rt, SEMCl (5.5 g, 33.0 mmol) was added. After addition, the reaction solution was heated at 80° C. for 1 h. The resulting reaction solution was poured into cold water and then extracted with EtOAc (100 mL×3). The combined organic layer was washed with brine, dried over Na₂SO₄ and concentrated under reduced pressure. The residue was purified by flash chromatography (eluting with PE/EtOAc=70:30 to 40:60) to give ethyl 2-(4-oxo-5-((2-(trimethylsilyl)ethoxy)methyl)-4,5-dihydro-1H-pyrrolo[2,3-d]pyridazin-1-yl)acetate 1105 (2 g, 95% purity, 81% yield) as an off-white solid.

LCMS (ESI) calcd for C₁₆H₂₅N₃O₄Si [M+H]⁺ m/z 352.17, found 352.

### Preparation of 1-(2-hydroxyethyl)-5-((2-(trimethylsilyl)ethoxy)methyl)-1,5-dihydro-4H-pyrrolo[2,3-d]pyridazin-4-one (1106)

To a suspension of LiAlH₄ (0.31 g, 8.1 mmol) in THE (35 mL), ethyl 2-(4-oxo-5-((2-(trimethylsilyl)ethoxy)methyl)-4,5-dihydro-1H-pyrrolo[2,3-d]pyridazin-1-yl)acetate 1105 (1.9 g, 5.4 mmol) in THF (15 mL) was added dropwise under N₂ atmosphere, during which the temperature was kept at 0-5° C. The reaction mixture was stirred at this temperature for a further 1 h. After the dropwise addition of water (0.3 mL) and 13% aq. NaOH (0.6 mL) successively, the mixture was stirred for an additional 30 min. The resulting mixture was filtered through diatomaceous earth and the filter cake was thoroughly washed with DCM. The filtrate was concentrated to dryness under reduced pressure. The residue was purified by flash chromatography (eluting with DCM/MeOH=100:0 to 90:10) to obtain 1-(2-hydroxyethyl)-5-((2-(trimethylsilyl)ethoxy)methyl)-1,5-dihydro-4H-pyrrolo[2,3-d]pyridazin-4-one 1106 (1 g, 85% purity, 59% yield) as a yellow solid.

LCMS (ESI) calcd for C₁₄H₂₃N₃O₃Si [M+H]⁺ m/z 310.16, found 310.

### Preparation of tert-butyl 3-(2-(4-oxo-5-((2-(trimethylsilyl)ethoxy)methyl)-4,5-dihydro-1H-pyrrolo[2,3-d]pyridazin-1-yl)ethoxy)propanoate (1107)

To a solution of 1-(2-hydroxyethyl)-5-((2-(trimethylsilyl)ethoxy)methyl)-1,5-dihydro-4H-pyrrolo[2,3-d]pyridazin-4-one 1106 (500 mg, 1.61 mmol, 1 eq.) in THE (50 mL), Na (74 mg, 3.22 mmol, 2 eq.) cut in pieces was added at rt. After stirring for 30 min, tert-butyl acrylate (619 mg, 4.83 mmol, 3 eq.) was added in one portion. The reaction mixture was stirred at rt for an additional 5 h. The reaction solution was sucked out (the rest of the Na was suspended in fresh THE and quenched with EtOH and then H₂O) and poured into cold water, and then extracted with EtOAc (30 mL×3). The combined organic layer was washed with brine, dried over Na₂SO₄ and concentrated under reduced pressure. The residue was purified by flash chromatography (eluting with petroleum ether/EtOAc=60:40 to 30:70) to give tert-butyl 3-(2-(4-oxo-5-((2-(trimethylsilyl)ethoxy)methyl)-4,5-dihydro-1H-pyrrolo[2,3-d]pyridazin-1-yl)ethoxy)propanoate 1107 (530 mg, 95% purity, 71% yield) as a colorless oil.

LCMS (ESI) calcd for C₂₁H₃₅N₃O₅Si [M+H]⁺ m/z 438.24, found 438

### Preparation of 3-(2-(4-oxo-4,5-dihydro-1H-pyrrolo[2,3-d]pyridazin-1-yl)ethoxy)propanoic acid (1108)

A solution of tert-butyl 3-(2-(4-oxo-5-((2-(trimethylsilyl)ethoxy)methyl)-4,5-dihydro-1H-pyrrolo[2,3-d]pyridazin-1-yl)ethoxy)propanoate 1107 (530 mg, 1.21 mmol) in HCl-Dioxane (4 M, 30 mL) was stirred at rt for 16 h under N₂ atmosphere. The resulting reaction mixture was evaporated under reduced pressure to afford 3-(2-(4-oxo-4,5-dihydro-1H-pyrrolo[2,3-d]pyridazin-1-yl)ethoxy)propanoic acid 1108 (300 mg, 85% purity, 84% yield) as a yellow solid.

LCMS (ESI) calcd for C₁₁H₁₃N₃O₄ [M+H]⁺ m/z 252.10, found 252.

### Preparation of 1-(2-(3-oxo-3-(4-(5-(trifluoromethyl)pyrimidin-2-yl)piperazin-1-yl)propoxy)ethyl)-1,5-dihydro-4H-pyrrolo[2,3-d]pyridazin-4-one (10)

To a solution of 3-(2-(4-oxo-4,5-dihydro-1H-pyrrolo[2,3-d]pyridazin-1-yl)ethoxy)propanoic acid 1108 (150 mg, 0.60 mmol) in MeCN (10 mL) was added 2-methylimidazole (123 mg, 1.49 mmol), TCFH (201 mg, 0.71 mmol) and 2-(piperazin-1-yl)-5-(trifluoromethyl)pyrimidine hydrochloride (209 mg, 0.78 mmol) at room temperature successively. The mixture was kept stirring at room temperature for 16 h. The resulting mixture was diluted with water (10 mL) and extracted with DCM (30 mL×3). The combined organic layer was dried over Na₂SO₄ and concentrated in vacuo. The residue was purified by flash chromatography (eluting with DCM/MeOH=100:0 to 90:10) and pre-HPLC (columns: Gemini 5 um C18 150×21.2 mm, mobile phase: ACN-H₂O (0.1% FA), gradient: 30-70) to give 1-(2-(3-oxo-3-(4-(5-(trifluoromethyl)pyrimidin-2-yl)piperazin-1-yl)propoxy)ethyl)-1,5-dihydro-4H-pyrrolo[2,3-d]pyridazin-4-one 10 (30.9 mg, 99% purity, 10% yield) as a white solid. ¹H NMR (400 MHz, DMSO-d₆, ppm) δ: 12.23 (s, 1H), 8.74 (s, 2H), 8.34 (s, 1H), 7.41 (d, J=2.8 Hz, 1H), 6.61 (d, J=2.8 Hz, 1H), 4.38 (t, J=4.8 Hz, 2H), 3.83-3.75 (m, 4H), 3.72 (t, J=4.8 Hz, 2H), 3.63 (t, J=6.4 Hz, 2H), 3.55-3.46 (m, 4H), 2.55-2.53 (m, 2H).

LCMS (ESI) calcd for C₂₀H₂₂F₃N₇O₃[M+H]⁺ m/z 466.18, found 466.

### 3. Synthesis of the enantiomers of 5-((1-(2-oxo-2-(4-(5-(trifluoromethyl)pyrimidin-2-yl)piperazin-1-yl)ethoxy)propan-2-yl)oxy)phthalazin-1(2H)-one (Compound 33)

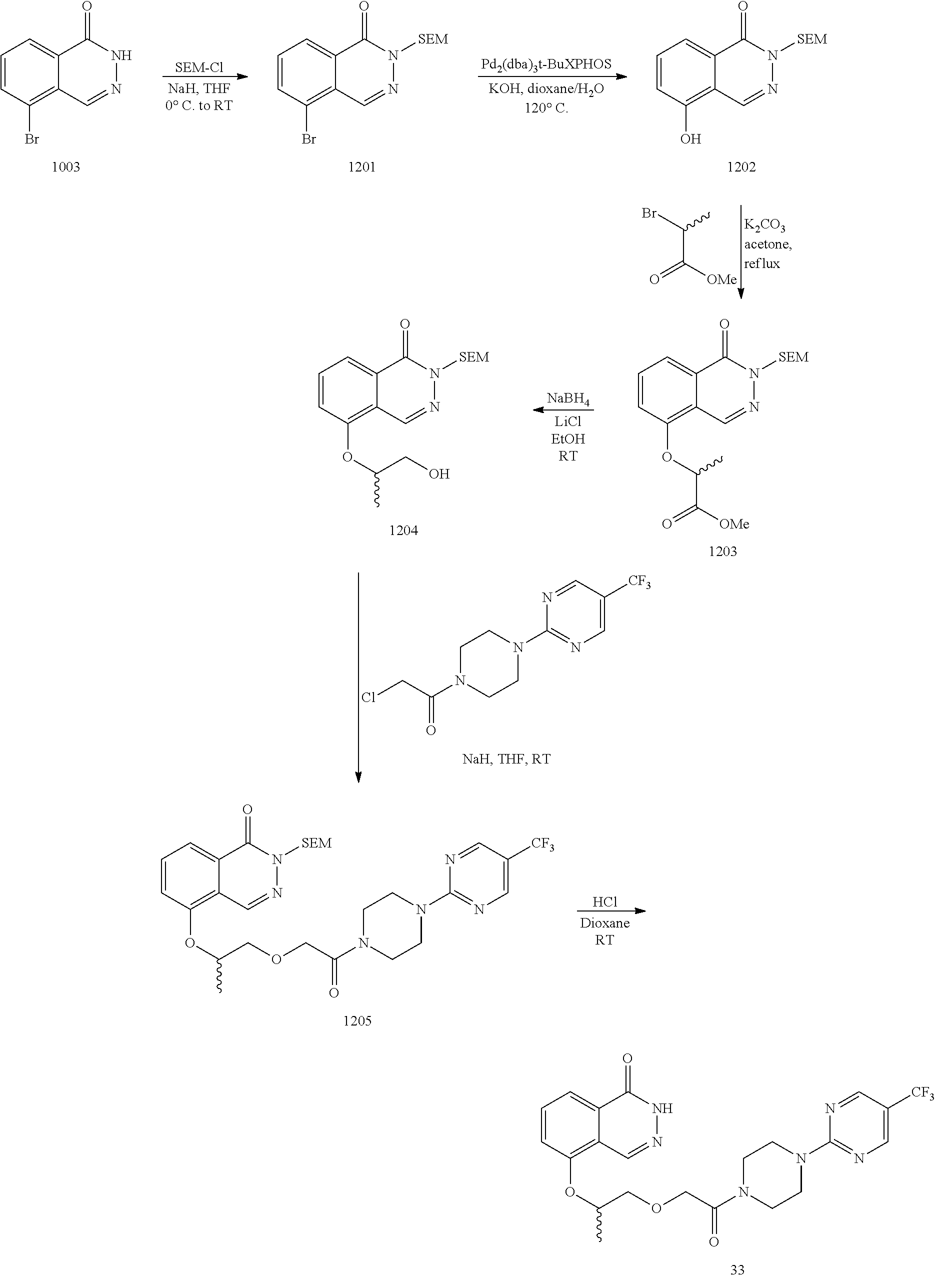

### Preparation of 5-bromo-2-((2-(trimethylsilyl)ethoxy)methyl)phthalazin-1(2H)-one (1201)

To a stirred suspension of NaH (60%, 0.78 g, 19.6 mmol) in THE (60 mL) was added 5-bromophthalazin-1(2H)-one 1003 (2.2 g, 9.8 mmol) in portions under nitrogen at 0° C. and then stirred for additional 10 min. After adding SEM-Cl (2.45 g, 14.7 mmol,), the reaction mixture was warmed to rt and stir for another 16 h. The resulting reaction mixture was poured into cold water (30 mL) and then extracted with EtOAc (50 mL×3). The combined organic layer was washed with brine, dried over anhydrous sodium sulfate and concentrated under reduced pressure. Purification by flash chromatography (eluting with petroleum ether/EtOAc=70:30 to 40:60) provided 5-bromo-2-((2-(trimethylsilyl)ethoxy)methyl)phthalazin-1(2H)-one 1201 (1.3 g, 95% purity, 35% yield) as a light yellow oil.

LCMS (ESI) calcd for C₁₄H₁₉BrN₂O₂Si [M+H]⁺ m/z 355.05, found 355, 357

### Preparation of 5-hydroxy-2-((2-(trimethylsilyl)ethoxy)methyl)phthalazin-1(2H)-one (1202)

To a solution of 5-bromo-2-((2 (trimethylsilyl)ethoxy)methyl)phthalazin-1(2H)-one 1201 (500 mg, 1.41 mmol) in dioxane/H₂O (10 mL, 1:1) was added Pd₂(dba)₃ (64 mg, 0.07 mmol), t-BuXPHOS (60 mg, 0.14 mmol) and KOH (240 mg, 4.23 mmol). The reaction mixture was stirred at 120° C. for 3 h. The reaction mixture was concentrated in vacuo. The residue was purified by silica gel column chromatography (eluting with petroleum ether/EtOAc=100:0 to 90:10) to afford 5-hydroxy-2-((2 (trimethylsilyl)ethoxy) methyl)phthalazin-1(2H)-one 1202 (321 mg, 90% purity, 78% yield) as a yellow solid.

LCMS (ESI) calcd for C₁₄H₂₀N₂O₃Si [M+H]⁺ m/z 293.13, found 293.

### Preparation of methyl 2-((1-oxo-2-((2-(trimethylsilyl)ethoxy)methyl)-1,2-dihydrophthalazin-5-yl)oxy)propanoate (1203)

To a solution of 5-hydroxy-2-((2-(trimethylsilyl)ethoxy)methyl)phthalazin-1(2H)-one 1202 (200 mg, 0.685 mmol) in acetone (5 mL) were added K₂CO₃ (171 mg, 2.058 mmol) and methyl 2-bromopropanoate (235 mg, 1.029 mmol). The reaction mixture was stirred at 80° C. for 3 h. The reaction solution was concentrated under reduced pressure. The residue was purified by silica gel column (eluting with petroleum ether/EtOAc=95:5 to 75:5) to obtain methyl 2-((1-oxo-2-((2-(trimethylsilyl)ethoxy)methyl)-1,2-dihydrophthalazin-5-yl)oxy)propanoate 1203 (246 mg, 90% purity, 95% yield) as yellow solid.

LCMS (ESI) calcd for C₁₈H₂₆N₂O₅Si [M+H]⁺ m/z 379.17, found 379.

### Preparation of 5-((1-hydroxypropan-2-yl)oxy)-2-((2-(trimethylsilyl)ethoxy)methyl)phthalazin-1(2H)-one (1204)

To a solution of methyl 2-((1-oxo-2-((2-(trimethylsilyl)ethoxy)methyl)-1,2-dihydrophthalazin-5-yl)oxy)propanoate 1203 (500 mg, 1.322 mmol) in EtOH (5 mL) was added LiCl (224 mg, 5.291 mmol). The reaction mixture was stirred at rt for 1 h. Then NaBH₄ (200 mg, 5.291 mmol) was added in portions. Then the reaction mixture was stirred at rt overnight. The resulting mixture was quenched with water and extracted with DCM (10 mL×3). The organic phase was concentrated under reduced pressure to obtain 5-((1-hydroxypropan-2-yl)oxy)-2-((2-(trimethylsilyl)ethoxy)methyl)phthalazin-1(2H)-one 1204 (460 mg, 90% purity, 99% yield) as a white solid.

LCMS (ESI) calcd for C₁₇H₂₆N₂O₄Si [M+H]⁺ m/z 351.17, found 351.

### Preparation of 5-((1-(2-oxo-2-(4-(5-(trifluoromethyl)pyrimidin-2-yl)piperazin-1-yl)ethoxy)propan-2-yl)oxy)-2-((2-(trimethylsilyl)ethoxy)methyl)phthalazin-1(2H)-one (1205)

To a solution of 5-((1-hydroxypropan-2-yl)oxy)-2-((2-(trimethylsilyl)ethoxy)methyl)phthalazin-1(2H)-one 1204 (200 mg, 0.571 mmol) in THE (2 mL) was added NaH (60 wt %, 46 mg, 1.143 mmol) at 0° C. The reaction mixture was stirred at rt for 0.5 h, then to the mixture 2-chloro-1-(4-(5-(trifluoromethyl)pyrimidin-2-yl)piperazin-1-yl)ethan-1-one (211 mg, 0.685 mmol) was added in one portion at rt and stirred at rt overnight. The reaction solution was concentrated under reduced pressure. The residue was purified by silica gel column (eluting with DCM/MeOH=95:5) to obtain 5-((1-(2-oxo-2-(4-(5-(trifluoromethyl)pyrimidin-2-yl)piperazin-1-yl)ethoxy)propan-2-yl)oxy)-2-((2-(trimethylsilyl)ethoxy)methyl)phthalazin-1(2H)-one 1205 (170 mg, 90% purity, 48% yield) as white solid.

LCMS (ESI) calcd for C₂₈H₃₇F₃N₆O₅Si [M+H]⁺ m/z 623.25, found 623.

### Preparation of 5-((1-(2-oxo-2-(4-(5-(trifluoromethyl)pyrimidin-2-yl)piperazin-1-yl)ethoxy)propan-2-yl)oxy)phthalazin-1(2H)-one (33 racemate)

5-((1-(2-oxo-2-(4-(5-(trifluoromethyl)pyrimidin-2-yl)piperazin-1-yl)ethoxy)propan-2-yl)oxy)-2-((2-(trimethylsilyl)ethoxy)methyl)phthalazin-1(2H)-one 1205 (150 mg, 0.241 mmol) was dissolved in HCl-dioxane (4 M, 4 mL). The reaction mixture was stirred at rt overnight under N₂. The solvent was removed and the residue was purified by prep-HPLC (columns: Gemini 5 um C18 150×21.2 mm, mobile phase: ACN-H₂O (0.1% FA), gradient: 25-65) to give 5-((1-(2-oxo-2-(4-(5-(trifluoromethyl)pyrimidin-2-yl)piperazin-1-yl)ethoxy)propan-2-yl)oxy)phthalazin-1(2H)-one 33 racemate (84 mg, 97% purity, 71% yield) as a white solid.

LCMS (ESI) calcd for C₂₂H₂₃F₃N₆O₄ [M+H]⁺ m/z 493.18, found 493.

### Chiral resolution of 5-((1-(2-oxo-2-(4-(5-(trifluoromethyl)pyrimidin-2-yl)piperazin-1-yl)ethoxy)propan-2-yl)oxy)phthalazin-1(2H)-one (33)

Compound 33 (racemate) was separated by SFC (Column: Daicel CHIRALPAK OJ-H 250 mm×20 mm I.D., 5 mm; Mobile phase: CO₂/MeOH[0.2% (NH₃)]=70/30) and concentrated under reduced pressure to afford the first fraction as Compound 33 enantiomer 1 (26.7 mg, 99% purity, ee %: 100, white solid) and the second fraction as Compound 33 enantiomer 2 (25 mg, 99% purity, ee %: 100, white solid)

### Compound 33 Enantiomer 1

¹H NMR (400 MHz, DMSO-d₆, ppm) δ: 12.67 (s, 1H), 8.71 (s, 2H), 8.44 (s, 1H), 7.82-7.66 (m, 2H), 7.56 (d, J=6.9 Hz, 1H), 4.90 (dd, J=10.8, 5.6 Hz, 1H), 4.39-4.20 (m, 2H), 3.90-3.64 (m, 6H), 3.48 (d, J=18.9 Hz, 4H), 1.33 (d, J=6.2 Hz, 3H).

LCMS (ESI) calcd for C₂₂H₂₃F₃N₆O₄ [M+H]⁺ m/z 493.18, found 493.

### Compound 33 Enantiomer 2

¹H NMR (400 MHz, DMSO-d₆, ppm) δ: 12.67 (s, 1H), 8.71 (s, 2H), 8.44 (s, 1H), 7.79-7.64 (m, 2H), 7.56 (dd, J=7.0, 2.0 Hz, 1H), 4.90 (dd, J=10.8, 5.7 Hz, 1H), 4.39-4.16 (m, 2H), 3.99-3.63 (m, 6H), 3.48 (d, J=19.2 Hz, 4H), 1.33 (d, J=6.2 Hz, 3H).

LCMS (ESI) calcd for C₂₂H₃F₃N₆O₄ [M+H]⁺ m/z 493.18, found 493.

### 4. Preparation of 1-(2-(3-oxo-3-(4-(5-(trifluoromethyl)pyrimidin-2-yl)piperazin-1-yl)propoxy) ethyl)-1,5-dihydro-4H-pyrazolo[3,4-d]pyridazin-4-one (Compound 50)

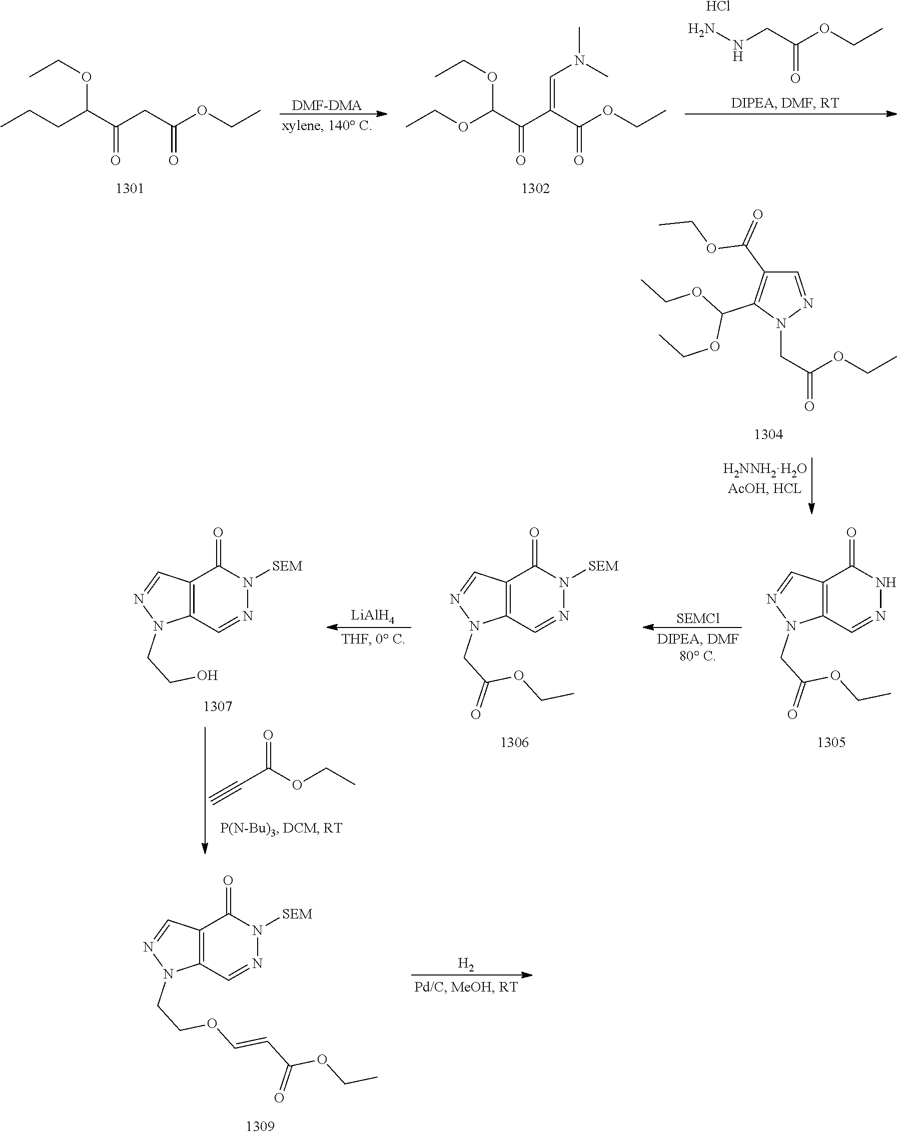

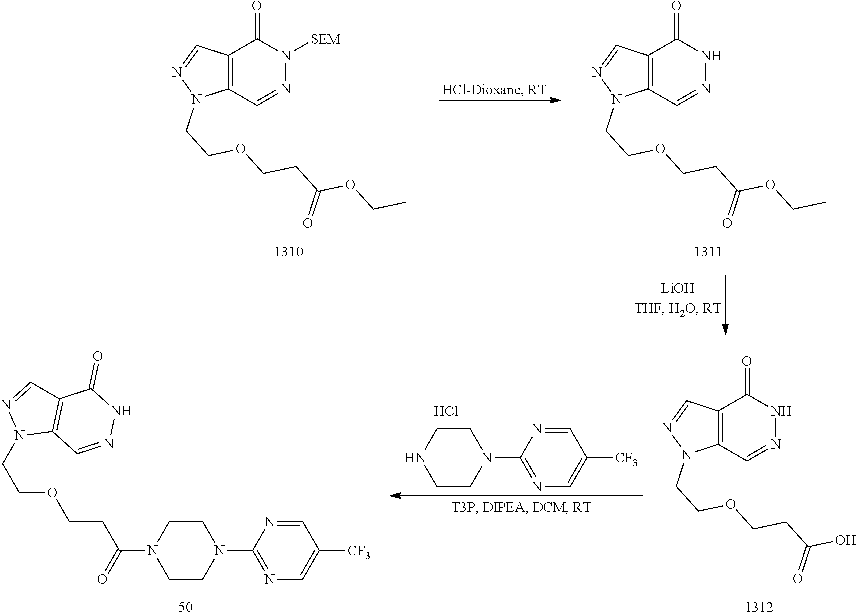

### Preparation of ethyl (Z)-2-((dimethylamino)methylene)-4,4-diethoxy-3-oxobutanoate (1302)

To a solution of ethyl 4,4-diethoxy-3-oxobutanoate 1301 (4 g, 0.0183 mol) in xylene (40 mL), DMF-DMA (4.36 g, 0.0366 mol) was added. The mixture was stirred at 140° C. for 2 h. After cooling to room temperature, the reaction mixture was poured into cold water and then extracted with EtOAc (50 mL×4). The combined organic layer was washed three times with brine solution, dried over Na₂SO₄ and concentrated under reduced pressure to give ethyl (Z)-2-((dimethylamino)methylene)-4,4-diethoxy-3-oxobutanoate 1302 (4 g, 60% purity, 48% yield) as a brown liquid which was used directly in the next step.

LCMS (ESI) calcd for C₁₃H₂₃NO₅ [M+H]⁺ m/z 274.16, found 274.

### Preparation of ethyl 5-(diethoxymethyl)-1-(2-ethoxy-2-oxoethyl)-1H-pyrazole-4-carboxylate (1304)

To a stirred solution of ethyl (Z)-2-((dimethylamino)methylene)-4,4-diethoxy-3-oxobutanoate 1302 (4 g, 0.0146 mol) and ethyl aminoglycinate hydrochloride 1303 (2.23 g, 0.0146 mmol) in DMF (40 mL) was added DIPEA (5.66 g, 0.0438 mmol) at room temperature. The reaction mixture was stirred at rt for 2 h. After the reaction completed, the reaction mixture was poured into cold water and then extracted with EtOAc (50 mL×4). The combined organic layer was washed three times with brine solution, dried over Na₂SO₄ and concentrated under reduced pressure. The residue was purified by flash chromatography (eluting with PE/EtOAc=100:0 to 50:50) to afford ethyl 5-(diethoxymethyl)-1-(2-ethoxy-2-oxoethyl)-1H-pyrazole-4-carboxylate 1304 (3.2 g, 90% purity, 60% yield) as a yellow liquid.

LCMS (ESI) calcd for C₁₅H₂₄N₂O₆ [M+H]⁺ m/z 329.17, found 329.

### Preparation of ethyl 2-(4-oxo-4,5-dihydro-1H-pyrazolo[3,4-d]pyridazin-1-yl)acetate (1305)

To a solution of ethyl 5-(diethoxymethyl)-1-(2-ethoxy-2-oxoethyl)-1H-pyrazole-4-carboxylate 1304 (3.2 g, 0.0097 mol) in AcOH (50 mL) were added con. HCl (0.18 g, 0.0048 mmol) and H₂NNH₂·H₂O (80% wt, 0.61 g, 0.0097 mmol). The mixture was stirred at 100° C. for 2 h. AcOH was removed under reduced pressure. The residue was diluted with water and extracted with DCM (30 mL×3). The combined organic layer was dried over Na₂SO₄ and concentrated under reduced pressure. The crude product was purified by flash chromatography (eluting with DCM/MeOH=90:10 to 80:20) to give ethyl 2-(4-oxo-4,5-dihydro-1H-pyrazolo[3,4-d]pyridazin-1-yl)acetate 1305 (0.7 g, 90% purity, 28% yield) as a white solid.

LCMS (ESI) calcd for C₉H₁₀N₄O₃[M+H]⁺ m/z 223.08, found 223.

### Preparation of ethyl 2-(4-oxo-5-((2-(trimethylsilyl)ethoxy)methyl)-4,5-dihydro-1H-pyrazolo[3,4-d]pyridazin-1-yl)acetate (1306)

To a solution of ethyl 2-(4-oxo-4,5-dihydro-1H-pyrazolo[3,4-d]pyridazin-1-yl)acetate 1305 (730 mg, 3.285 mmol) in DMF (20 mL) were added SEMCl (821 mg, 4.928 mmol) and DIPEA (1273.78 mg, 9.856 mol) at rt. Then the reaction mixture was stirred 80° C. for 2 h. After cooling to room temperature, the reaction mixture was poured into cold water and then extracted with EtOAc (50 mL×4). The combined organic layer was washed three times with brine solution, dried over Na₂SO₄ and concentrated under reduced pressure. The residue was purified by flash chromatography (eluting with PE/EtOAc=100:0 to 50:50) to afford ethyl 2-(4-oxo-5-((2-(trimethylsilyl)ethoxy)methyl)-4,5-dihydro-1H-pyrazolo [3,4-d]pyridazin-1-yl)acetate 1306 (700 mg, 95% purity, 57% yield) as a white solid.

LCMS (ESI) calcd for C₁₅H₂₄N₄O₄Si [M+H]⁺ m/z 353.16, found 353.

### Preparation of 1-(2-hydroxyethyl)-5-((2-(trimethylsilyl)ethoxy)methyl)-1,5-dihydro-4H-pyrazolo[3,4-d]pyridazin-4-one (1307)

To a stirred solution of ethyl 2-(4-oxo-5-((2-(trimethylsilyl)ethoxy)methyl)-4,5-dihydro-1H-pyrazolo [3,4-d]pyridazin-1-yl)acetate 1306 (690 mg, 1.952 mmol) in 20 ml of THF was added LiAlH₄ (114 mg, 2.928 mmol) in portions at 0° C. The mixture was stirred at 0° C. for 5 min, then the reaction mixture was quenched with H₂O (0.1 mL), NaOH (15% wt in water, 0.3 mL) and H₂O (0.3 mL) successively. The resulting mixture was filtered through diatomaceous earth and washed with DCM several times. The filtrate was concentrated under reduced pressure to afford 1-(2-hydroxyethyl)-5-((2-(trimethylsilyl)ethoxy)methyl)-1,5-dihydro-4H-pyrazolo[3,4-d]pyridazin-4-one 1307 (600 mg, 90% purity, 88% yield) as a yellow oil.

LCMS (ESI) calcd for C₁₃H₂₂N₄O₃Si [M+H]⁺ m/z 311.15, found 311.

### Preparation of ethyl (E)-3-(2-(4-oxo-5-((2-(trimethylsilyl)ethoxy)methyl)-4,5-dihydro-1H-pyrazolo[3,4-d]pyridazin-1-yl)ethoxy)acrylate (1309)

To a solution of 1-(2-hydroxyethyl)-5-((2-(trimethylsilyl)ethoxy)methyl)-1,5-dihydro-4H-pyrazolo[3,4-d]pyridazin-4-one 1307 (300 mg, 0.963 mmol) in DCM (15 mL) were added ethyl propiolate 1308 (94 mg, 0.963 mmol) and P(n-Bu)₃ (19 mg, 0.0963 mmol) successively at room temperature. The solution was then stirred at rt for 1 h. After reaction completed, the reaction mixture was concentrated under reduced pressure. The residue was purified by flash chromatography (eluting with PE/EtOAc=70:30 to 30:70) to give ethyl (E)-3-(2-(4-oxo-5-((2-(trimethylsilyl)ethoxy)methyl)-4,5-dihydro-1H-pyrazolo[3,4-d]pyridazin-1-yl)ethoxy)acrylate 1309 (200 mg, 95% purity, 48% yield) as a yellow oil.

LCMS (ESI) calcd for C₁₈H₂₈N₄O₅Si [M+Na]⁺ m/z 431.17, found 431.

### Preparation of ethyl 3-(2-(4-oxo-5-((2-(trimethylsilyl)ethoxy)methyl)-4,5-dihydro-1H-pyrazolo[3,4-d]pyridazin-1-yl)ethoxy)propanoate (1310)

To a solution of ethyl (E)-3-(2-(4-oxo-5-((2-(trimethylsilyl)ethoxy)methyl)-4,5-dihydro-1H-pyrazolo[3,4-d]pyridazin-1-yl)ethoxy)acrylate 1309 (200 mg, 0.488 mmol) in MeOH (10 mL) was added Pd/C (17 mg) at room temperature. The reaction mixture was stirred at rt for 16 h under H₂ atmosphere. The reaction solution was filtered through diatomaceous earth. Then the filtrate was concentrated under reduced pressure to obtain ethyl 3-(2-(4-oxo-5-((2-(trimethylsilyl)ethoxy) methyl)-4,5-dihydro-1H-pyrazolo[3,4-d]pyridazin-1-yl)ethoxy)propanoate 1310 (190 mg, 95% purity, 89% yield) as yellow oil.

LCMS (ESI) calcd for C₁₈H₃₀N₄O₅Si [M+H]⁺ m/z 411.20, found 411.

### Preparation of ethyl 3-(2-(4-oxo-4,5-dihydro-1H-pyrazolo[3,4-d]pyridazin-1-yl)ethoxy) propanoate (1311)

To a solution of ethyl 3-(2-(4-oxo-5-((2-(trimethylsilyl)ethoxy)methyl)-4,5-dihydro-1H-pyrazolo[3,4-d]pyridazin-1-yl)ethoxy)propanoate 1310 (190 mg, 0.4617 mmol) in HCl-Dioxane (4 M, 20 mL) was added at rt and the reaction was stirred for additional 16 h. The resulting reaction mixture was evaporated under reduced pressure to afford ethyl 3-(2-(4-oxo-4,5-dihydro-1H-pyrazolo[3,4-d]pyridazin-1-yl)ethoxy) propanoate 1311 (200 mg, 60% purity, 92% yield) as a yellow oil.

LCMS (ESI) calcd for C₁₂H₁₆N₄O₄ [M+H]⁺ m/z 281.12, found 281.

### Preparation of 3-(2-(4-oxo-4,5-dihydro-1H-pyrazolo[3,4-d]pyridazin-1-yl)ethoxy)propanoic acid (1312)

To a solution of ethyl 3-(2-(4-oxo-4,5-dihydro-1H-pyrazolo[3,4-d]pyridazin-1-yl)ethoxy)propanoate 1311 (200 mg, 0.7136 mmol) in THF/H₂O (20 mL, 3:1) was added LiOH (51 mg, 2.141 mmol). The mixture was stirred at rt for 1 h. THF was removed under reduced pressure and the aqueous phase was acidified with 1 M aq. HCl to pH=4˜5. The residue was purified by C18 columns (mobile phase: ACN-H₂O (0.1% FA), gradient: 40-60) to give 3-(2-(4-oxo-4,5-dihydro-1H-pyrazolo[3,4-d]pyridazin-1-yl)ethoxy)propanoic acid 1312 (35 mg, 97% purity, 18% yield) as a white solid.

LCMS (ESI) calcd for C₁₀H₁₂N₄O₄ [M+H]⁺ m/z 253.09, found 253.

### Preparation of 1-(2-(3-oxo-3-(4-(5-(trifluoromethyl)pyrimidin-2-yl)piperazin-1-yl)propoxy) ethyl)-1,5-dihydro-4H-pyrazolo[3,4-d]pyridazin-4-one (Compound 50)

To a solution of 3-(2-(4-oxo-4,5-dihydro-1H-pyrazolo[3,4-d]pyridazin-1-yl)ethoxy)propanoic acid 1312 (35 mg, 0.139 mmol) in DCM (5 mL) were added 2-(piperazin-1-yl)-5-(trifluoromethyl) pyrimidine hydrochloride Int 3 (48 mg, 0.208 mmol), DIPEA (89 mg, 0.694 mmol), T3P (50% in EtOAc, 132 mg, 0.208 mmol) at room temperature successively. The mixture was kept stirring at room temperature for 1 h. The resulting mixture was diluted with water and extracted with DCM (30 mL×3). The combined organic layer was dried over Na₂SO₄ and concentrated under reduced pressure. The residue was purified by C18 column (mobile phase: ACN-H₂O (0.1% FA), gradient: 40-60) to give 1-(2-(3-oxo-3-(4-(5-(trifluoromethyl)pyrimidin-2-yl) piperazin-1-yl)propoxy) ethyl)-1,5-dihydro-4H-pyrazolo[3,4-d]pyridazin-4-one 50 (10 mg, 97% purity, 14% yield) as white solid.

¹H NMR (400 MHz, DMSO-d₆, ppm) δ: 12.51 (s, 1H), 8.73 (s, 2H), 8.50 (s, 1H), 8.22 (s, 1H), 4.60 (t, J=4.8 Hz, 2H), 3.84-3.71 (m, 6H), 3.61 (t, J=6.4 Hz, 2H), 3.52-3.43 (m, 4H), 2.48-2.46 (m, 2H).

LCMS (ESI) calcd for C₁₉H₂₁F₃N₈O₃[M+H]⁺ m/z 467.17, found 467.

### 5. Synthesis of 1-(2-(3-oxo-3-(4-(5-(trifluoromethyl)pyrimidin-2-yl)piperazin-1-yl)propoxy)ethyl)-2-(trifluoromethyl)-1,5-dihydro-4H-pyrrolo[2,3-d]pyridazin-4-one (Compound 60)

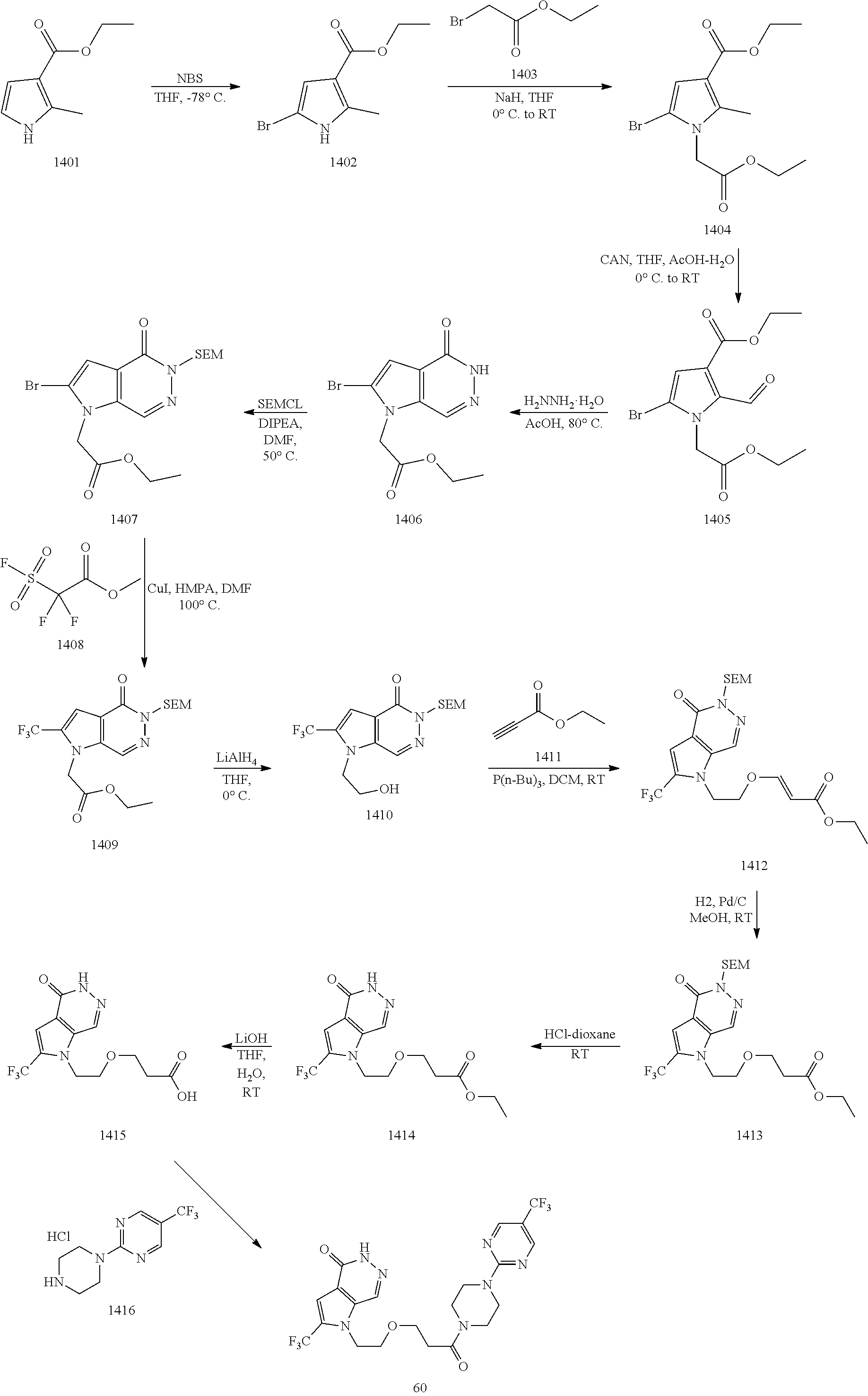

### Preparation of ethyl 5-bromo-1-(2-ethoxy-2-oxoethyl)-2-methyl-1H-pyrrole-3-carboxylate (1404)

To the solution of ethyl 2-methyl-1H-pyrrole-3-carboxylate 1401 (5.0 g, 32.64 mmol) in THE (300 mL) was added NBS (5.8 g, 32.64 mmol). The reaction mixture was stirred at −78° C. for 1 h. After LCMS showed that the 1402 formed completely, NaH (6.5 g, 163.20 mmol, 60% wt) was added to the reaction mixture. Ethyl 2-bromoacetate 1403 (6.5 g, 39.17 mmol) was added after the reaction mixture was stirred at 0° C. for 30 min. The reaction solution was stirred at rt for 16 h until 1402 was consumed completely. The reaction mixture was quenched with saturated aqueous NH₄Cl (20 mL). The aqueous layer was extracted with EtOAc (200 ml×2). The combined organic layers were concentrated under reduced pressure. The residue was purified by flash column chromatography (eluting with PE/EtOAc=85:15 to 70:30) to afford ethyl 5-bromo-1-(2-ethoxy-2-oxoethyl)-2-methyl-1H-pyrrole-3-carboxylate 1404 (9.29 g, 90% purity, 81% yield) as a white solid.

LCMS (ESI) calcd for C₁₂H₁₆BrNO₄ [M+H]⁺ m/z 318.03, found 318/320.

### Preparation of ethyl 5-bromo-1-(2-ethoxy-2-oxoethyl)-2-formyl-1H-pyrrole-3-carboxylate (1405)

Ethyl 5-bromo-1-(2-ethoxy-2-oxoethyl)-2-methyl-1H-pyrrole-3-carboxylate 1404 (4.0 g, 12.62 mmol) was dissolved in THE (50 mL) under stirring, followed by addition a solution of AcOH (50 mL) and H₂O (50 mL). The mixture was homogeneously stirred at 0° C. and ceric ammonium nitrate (CAN) was added (28 g, 50.47 mmol) in one portion. After stirring at rt for 1 h, the reaction mixture was poured into ice-water (200 mL) and stirred for another 30 min. The resulting solution was extracted with EtOAc (100 mL×3). The combined organic layers were washed with brine, dried over Na₂SO₄ and concentrated under reduced pressure. The residue was purified by flash chromatography (eluting with PE/EtOAc=100:0 to 80:20) to obtain ethyl 5-bromo-1-(2-ethoxy-2-oxoethyl)-2-formyl-1H-pyrrole-3-carboxylate 1405 (2.3 g, 90% purity, 49% yield) as a yellow solid.

LCMS (ESI) calcd for C₁₂H₁₄BrNO₅ [M+H]⁺ m/z 332.0, found 332/334.

### Preparation of ethyl 2-(2-bromo-4-oxo-4,5-dihydro-1H-pyrrolo[2,3-d]pyridazin-1-yl)acetate (1406)

To a solution of ethyl 5-bromo-1-(2-ethoxy-2-oxoethyl)-2-formyl-1H-pyrrole-3-carboxylate 1405 (2.0 g, 6.04 mmol) in AcOH (20 mL) was added H₂NNH₂·H₂O (80% wt, 1.2 g, 30.20 mmol) in one portion. The reaction mixture was heated with stirring at 80° C. for 1 h. The solvent was removed by evaporation under reduced pressure. The residue was purified by flash column chromatography (eluting with PE/EtOAc=40:60 to 20:80) to give ethyl 2-(2-bromo-4-oxo-4,5-dihydro-1H-pyrrolo[2,3-d]pyridazin-1-yl)acetate 1406 (900 mg, 90% purity, 45% yield) as a yellow solid.

LCMS (ESI) calcd for C₁₀H₁₀BrN₃O₃[M+H]⁺ m/z 299.99, found 300/302.

### Preparation of ethyl 2-(2-bromo-4-oxo-5-((2-(trimethylsilyl)ethoxy)methyl)-4,5-dihydro-1H-pyrrolo[2,3-d]pyridazin-1-yl)acetate (1407)

To a solution of ethyl 2-(2-bromo-4-oxo-4,5-dihydro-1H-pyrrolo[2,3-d]pyridazin-1-yl)acetate 1406 (1.88 g, 6.3 mmol) and DIPEA (4.10 g, 31.5 mmol) in DMF (15 mL) at rt, SEMCl (2.10 g, 12.6 mmol) was added. After completing of addition, the reaction solution was heated at 50° C. for 1 h. The resulting reaction solution was poured into cold water and then extracted with EtOAc (50 mL×3). The combined organic layer was washed with brine, dried over Na₂SO₄ and concentrated under reduced pressure. The residue was purified by flash chromatography (eluting with PE/EtOAc=70:30 to 40:60) to obtained ethyl 2-(2-bromo-4-oxo-5-((2-(trimethylsilyl)ethoxy) methyl)-4,5-dihydro-1H-pyrrolo[2,3-d]pyridazin-1-yl)acetate 1407 (2.5 g, 90% purity, 82% yield) as an off-white solid.

LCMS (ESI) calcd for C₁₆H₂₄BrN₃O₄Si [M+H]⁺ m/z 430.1, found 430/432.

### Preparation of ethyl 2-(4-oxo-2-(trifluoromethyl)-5-((2-(trimethylsilyl)ethoxy)methyl)-4,5-dihydro-1H-pyrrolo[2,3-d]pyridazin-1-yl)acetate (1409)

To a solution of ethyl 2-(2-bromo-4-oxo-5-((2-(trimethylsilyl)ethoxy)methyl)-4,5-dihydro-1H-pyrrolo[2,3-d]pyridazin-1-yl)acetate 1407 (1.10 g, 2.5 mmol) in DMF (20 mL) were added CuI (0.95 g, 5.1 mmol), HMPA (2.24 g, 12.5 mmol) and methyl 2,2-difluoro-2-(fluorosulfonyl)acetate 1408 (2.40 g, 12.5 mmol) successively. The reaction mixture was stirred at 100° C. for 48 h. The resulting reaction solution was poured into cold water and then extracted with EtOAc (50 mL×3). The combined organic layer was washed with brine, dried over Na₂SO₄ and concentrated under reduced pressure. The residue was purified by flash chromatography (eluting with PE/EtOAc=70:30 to 40:60) to obtain ethyl 2-(4-oxo-2-(trifluoromethyl)-5-((2-(trimethylsilyl)ethoxy)methyl)-4,5-dihydro-1H-pyrrolo[2,3-d]pyridazin-1-yl)acetate 1409 (455 mg, 90% purity, 40% yield) as an off-white solid.

LCMS (ESI) calcd for C₁₇H₂₄F₃N₃O₄Si [M+H]⁺ m/z 420.1, found 420.

### Preparation of 1-(2-hydroxyethyl)-2-(trifluoromethyl)-5-((2-(trimethylsilyl)ethoxy)methyl)-1,5-dihydro-4H-pyrrolo[2,3-d]pyridazin-4-one (1410)

To the solution of ethyl 2-(4-oxo-2-(trifluoromethyl)-5-((2-(trimethylsilyl)ethoxy)methyl)-4,5-dihydro-1H-pyrrolo[2,3-d]pyridazin-1-yl)acetate 1409 (450 mg, 1.07 mmol) in THE (10 mL) was added LiAlH₄ (81 mg, 2.14 mmol) in portions at 0° C. The reaction mixture was stirred at 0° C. for 0.5 h. The reaction mixture was quenched with water, the aqueous layer was extracted with EtOAc (50 mL×3). The combined organic layers were concentrated under reduced pressure. The residue was purified by flash column chromatography (eluting with PE/EtOAc=50:50 to 40:60) to afford 1-(2-hydroxyethyl)-2-(trifluoromethyl)-5-((2-(trimethylsilyl)ethoxy)methyl)-1,5-dihydro-4H-pyrrolo[2,3-d]pyridazin-4-one 1410 (297 mg, 90% purity, 66% yield) as a white solid.

LCMS (ESI) calcd for C₁₅H₂₂F₃N₃O₃Si [M+H]⁺ m/z 378.1, found 378.

### Preparation of ethyl (E)-3-(2-(4-oxo-2-(trifluoromethyl)-5-((2-(trimethylsilyl)ethoxy)methyl)-4,5-dihydro-1H-pyrrolo[2,3-d]pyridazin-1-yl)ethoxy)acrylate (1412)

To the solution of 1-(2-hydroxyethyl)-2-(trifluoromethyl)-5-((2-(trimethylsilyl)ethoxy)methyl)-1,5-dihydro-4H-pyrrolo[2,3-d]pyridazin-4-one 1410 (270 mg, 0.71 mmol) in DCM (10 mL) were added ethyl propiolate 1411 (70 mg, 0.71 mmol) and P(n-Bu)₃ (72 mg, 0.36 mmol) successively. The reaction mixture was stirred at room temperature for 2 h. The reaction mixture was diluted with DCM (20 mL) and water (20 mL). The aqueous layer was extracted with DCM (20 mL×2). The combined organic layers were dried over Na₂SO₄ and concentrated under reduced pressure. The residue was purified by flash column chromatography (eluting with PE/EtOAc=70:30 to 60:40) to afford ethyl (E)-3-(2-(4-oxo-2-(trifluoromethyl)-5-((2-(trimethylsilyl)ethoxy)methyl)-4,5-dihydro-1H-pyrrolo[2,3-d]pyridazin-1-yl)ethoxy)acrylate 1412 (302 mg, 90% purity, 83% yield) as a white solid.

LCMS (ESI) calcd for C₂₀H₂₈F₃N₃O₅Si [M+Na]⁺ m/z 498.2, found 498.

### Preparation of ethyl 3-(2-(4-oxo-2-(trifluoromethyl)-5-((2-(trimethylsilyl)ethoxy)methyl)-4,5-dihydro-1H-pyrrolo[2,3-d]pyridazin-1-yl)ethoxy)propanoate (1413)

A solution of ethyl (E)-3-(2-(4-oxo-2-(trifluoromethyl)-5-((2-(trimethylsilyl)ethoxy)methyl)-4,5-dihydro-1H-pyrrolo[2,3-d]pyridazin-1-yl)ethoxy)acrylate 1412 (300 mg, 0.63 mmol) and Pd/C (30 mg, 10% wt) in MeOH (15 mL) was stirred at rt for 1 h under H₂ atmosphere. The resulting solution was filtered through diatomaceous earth and the filter cake was washed with DCM (5 mL×4). The filtrate was concentrated under reduced pressure. The residue was purified by flash column chromatography (eluting with DCM/MeOH=97:3 to 95:5) to afford ethyl 3-(2-(4-oxo-2-(trifluoromethyl)-5-((2-(trimethylsilyl)ethoxy)methyl)-4,5-dihydro-1H-pyrrolo[2,3-d]pyridazin-1-yl)ethoxy)propanoate 1413 (238 mg, 90% purity, 71% yield) as an off-white solid.

LCMS (ESI) calcd for C₂₀H₃₀F₃N₃O₅Si [M+H]⁺ m/z 478.2, found 478.

### Preparation of ethyl 3-(2-(4-oxo-2-(trifluoromethyl)-4,5-dihydro-1H-pyrrolo[2,3-d]pyridazin-1-yl)ethoxy)propanoate (1414)

Dissolved ethyl 3-(2-(4-oxo-2-(trifluoromethyl)-5-((2-(trimethylsilyl)ethoxy)methyl)-4,5-dihydro-1H-pyrrolo[2,3-d]pyridazin-1-yl)ethoxy) propanoate 1413 (230 mg, 0.48 mmol) in HCl-dioxane (4 M, 15 mL). The reaction mixture was stirred at room temperature for 12 h. The resulting reaction mixture was evaporated under reduced pressure to give ethyl 3-(2-(4-oxo-2-(trifluoromethyl)-4,5-dihydro-1H-pyrrolo[2,3-d]pyridazin-1-yl)ethoxy)propanoate 1414 (180 mg, 90% purity, 97% yield) as a colorless oil.

LCMS (ESI) calcd for C₁₄H₁₆F₃N₃O₄[M+H]⁺ m/z 348.1, found 348.

### Preparation of 3-(2-(4-oxo-2-(trifluoromethyl)-4,5-dihydro-1H-pyrrolo[2,3-d]pyridazine-1-yl)ethoxy)propanoic acid (1415)

To a solution of ethyl 3-(2-(4-oxo-2-(trifluoromethyl)-4,5-dihydro-1H-pyrrolo[2,3-d]pyridazin-1-yl)ethoxy)propanoate 1414 (200 mg, 0.57 mmol) in THF/H₂O (15 mL, 1:1) was added LiOH (72 mg, 1.72 mmol). The mixture was stirred at room temperature for 2 h. THE was removed under reduced pressure and the aqueous phase was acidified with 1 M HCl aq. to pH=4˜5. The resulting solid was collected by filtration and dried in vacuo to obtain 3-(2-(4-oxo-2-(trifluoromethyl)-4,5-dihydro-1H-pyrrolo[2,3-d]pyridazine-1-yl)ethoxy)propanoic acid 1415 (82 mg, 90% purity, 40% yield) as a white solid.

LCMS (ESI) calcd for C₁₂H₁₂F₃N₃O₄ [M+H]⁺ m/z 320.0, found 320.

### Preparation of 1-(2-(3-oxo-3-(4-(5-(trifluoromethyl)pyrimidin-2-yl)piperazin-1-yl)propoxy) ethyl)-2-(trifluoromethyl)-1,5-dihydro-4H-pyrrolo[2,3-d]pyridazin-4-one (Compound 60)

To a solution 3-(2-(4-oxo-2-(trifluoromethyl)-4,5-dihydro-1H-pyrrolo[2,3-d]pyridazine-1-yl)ethoxy)propanoic acid 1415 (40 mg, 0.12 mmol), 2-(piperazin-1-yl)-5-(trifluoromethyl) pyrimidine hydrochloride 1416 (35 mg, 0.15 mmol) in DCM (5 mL) were added DIPEA (658 mg, 0.50 mmol), T3P (50% wt in EtOAc, 160 mg, 0.25 mmol) at rt successively. The reaction mixture was stirred at rt for 1 h. The reaction solution was quenched with water (20 mL) and extracted with DCM (20 mL×3). The organic phase was concentrated and purified by prep-HPLC (columns: Gemini 5 um C18 150×21.2 mm, mobile phase: ACN-H₂O (0.1% FA), gradient: 30-95) to obtain 1-(2-(3-oxo-3-(4-(5-(trifluoromethyl)pyrimidin-2-yl)piperazin-1-yl)propoxy)ethyl)-2-(trifluoromethyl)-1,5-dihydro-4H-pyrrolo[2,3-d]pyridazin-4-one 60 (23.4 mg, 100% purity, 35% yield) as a white solid.

¹H NMR (400 MHz, DMSO-d6, ppm) δ: 12.54 (s, 1H), 8.74 (d, J=0.8 Hz, 2H), 8.40 (s, 1H), 7.27 (s, 1H), 4.49 (t, J=5.0 Hz, 2H), 3.84-3.75 (m, 4H), 3.72 (t, J=5.0 Hz, 2H), 3.61 (t, J=6.4 Hz, 2H), 3.52-3.43 (m, 4H), 2.49-2.44 (m, 2H).

LCMS (ESI) calcd for C₂₁H₂₁F₆N₇O₃ [M+H]⁺ m/z 534.1, found 534.

### 6. Synthesis of 3-methyl-1-(2-(3-oxo-3-(4-(5-(trifluoromethyl)pyrimidin-2-yl)piperazin-1-yl)propoxy)ethyl)-2,3,4,6-tetrahydropyrido[2,3-d]pyridazin-5(1H)-one (Compounds 64 and 65)

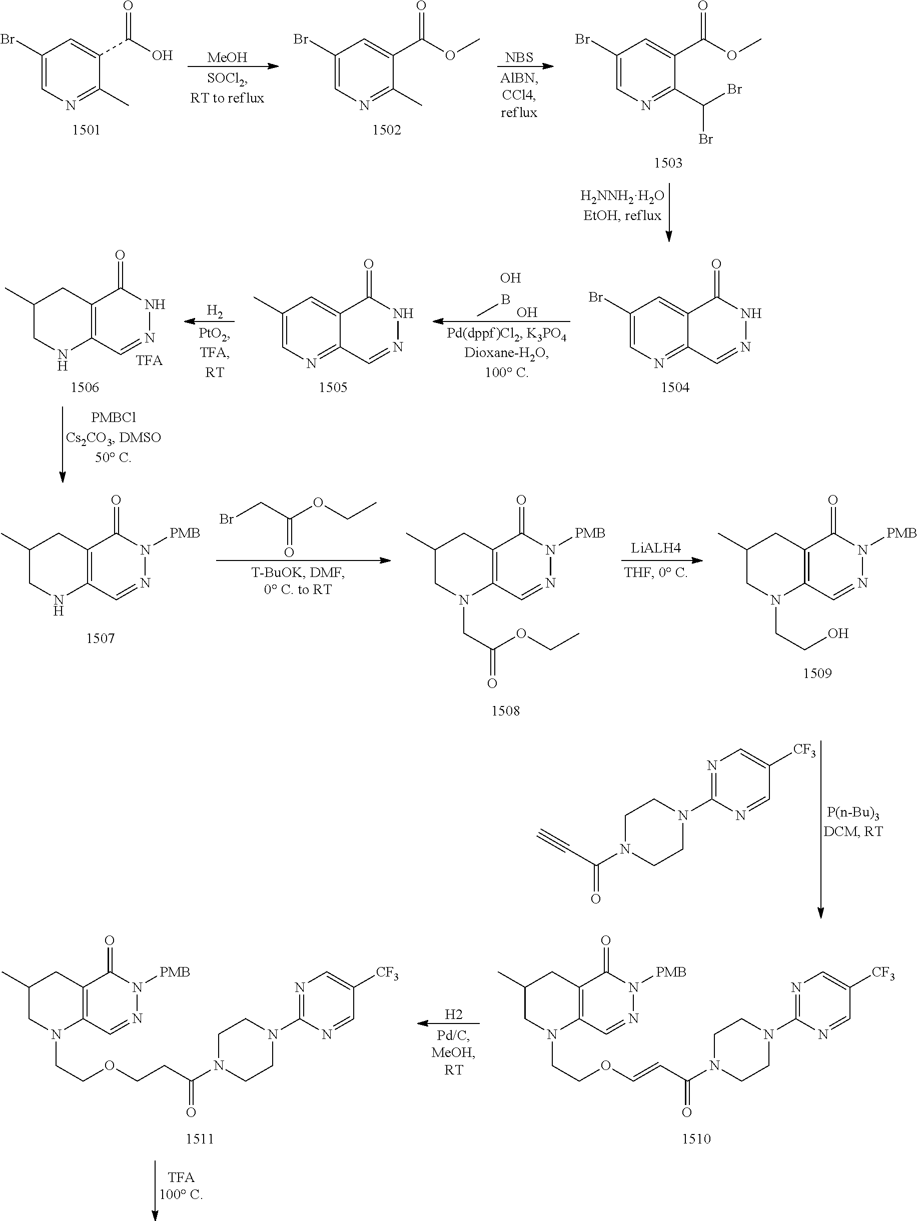

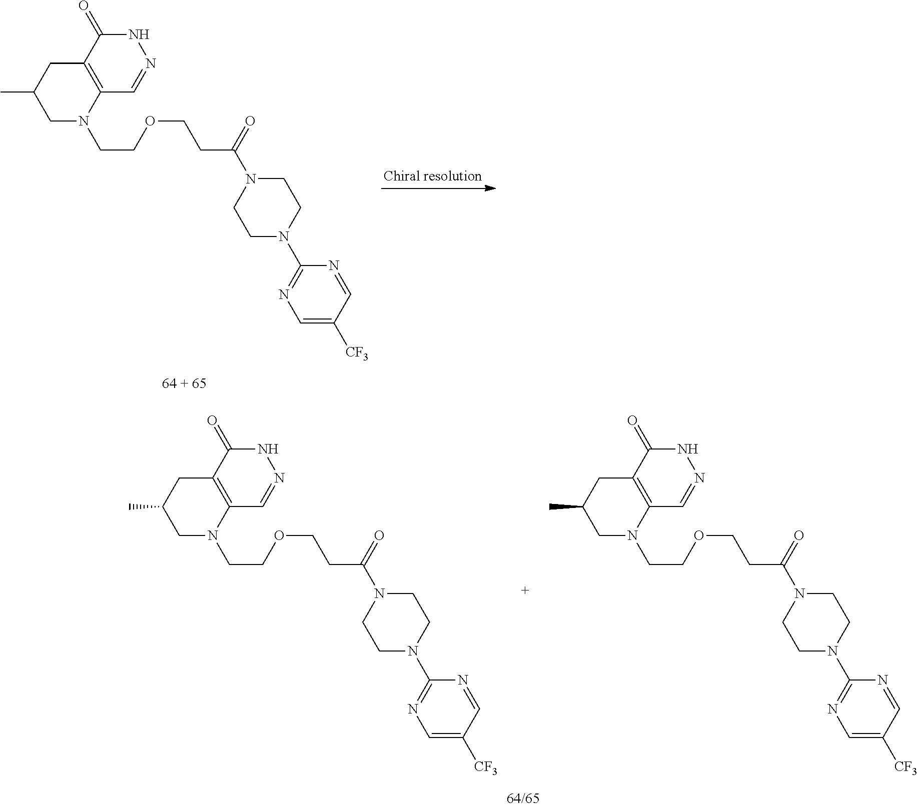 [SMILES: CC1Cc2c(cn[nH]c2=O)N(CCOCCC(=O)N2CCN(c3ncc(C(F)(F)F)cn3)CC2)C1 ; C[C@@H]1Cc2c(cn[nH]c2=O)N(CCOCCC(=O)N2CCN(c3ncc(C(F)(F)F)cn3)CC2)C1 ; C[C@H]1Cc2c(cn[nH]c2=O)N(CCOCCC(=O)N2CCN(c3ncc(C(F)(F)F)cn3)CC2)C1]

### Preparation of methyl 5-bromo-2-methylnicotinate (1502)

To a solution of 5-bromo-2-methylnicotinic acid 1501 (20 g, 92.4 mmol) in MeOH (300 mL) was added SOCl₂ (66 mL, 924 mmol) at 0° C. The resulting mixture was stirred at 80° C. for 16 h. After cooling to room temperature, the mixture was concentrated in vacuo. Water (100 mL) was added, and the mixture was extracted EtOAc (3×150 mL). The combined organic layers were dried (Na₂SO₄), concentrated in vacuo to obtain methyl 5-bromo-2-methylnicotinate 1502 (17.2 g, 90% purity, 73% yield) as colorless oil.

LCMS (ESI) calcd for C₈H₈BrNO₂ [M+H]⁺ m/z 229.98, found 230.

### Preparation of methyl 5-bromo-2-(dibromomethyl)nicotinate (1503)

To a solution of methyl 5-bromo-2-methylnicotinate 1502 (15 g, 65.1 mmol) in CCl₄ (350 mL were added NBS (46.4 g, 260.4 mmol) and AIBN (2.1 g, 13.0 mmol) at room temperature. The reaction mixture was stirred at 85° C. for 30 h. After cooling to room temperature, water (400 mL) was added and the mixture was extracted EtOAc (3×350 mL). The combined organic layers were dried (Na₂SO₄), concentrated in vacuo, and purified by flash chromatography (silica gel, eluting with PE/EtOAc=100:0 to 10:90) to obtain methyl 5-bromo-2-(dibromomethyl)nicotinate 1503 (12 g, 85% purity, 41% yield) as white solid.

LCMS (ESI) calcd for C₈H₆Br₃NO₂ [M+H]⁺ m/z 385.80, found 386.

### Preparation of 3-bromopyrido[2,3-d]pyridazin-5(6H)-one (1504)

To a solution of methyl 5-bromo-2-(dibromomethyl)nicotinate 1503 (10 g, 25.8 mmol) in AcOH (150 mL) was added NH₂NH₂·H₂O (6.46 g, 129 mmol) at room temperature. The resulting mixture was stirred at 100° C. for 10 h. After cooling to room temperature, the mixture was concentrated in vacuo. The residue was purified by flash chromatography (silica gel, eluting with PE/EtOAc=100:0 to 20:80) to obtain 3-bromopyrido[2,3-d]pyridazin-5(6H)-one 1504 (5 g, 80% purity, 69% yield) as white solid.

LCMS (ESI) calcd for C₇H₄BrN₃O [M+H]⁺ m/z 225.96, found 226.

### Preparation of 3-methylpyrido[2,3-d]pyridazin-5(6H)-one (1505)

To a solution of 3-bromopyrido[2,3-d]pyridazin-5(6H)-one 1504 (4.8 g, 21.2 mmol) in Dioxane-H₂O (60 mL, v/v=3:1) were added K₃PO₄ (27 g, 127.2 mmol), methylboronic acid (5.08 g, 84.8 mmol) and Pd(dppf)Cl₂ (1.56 g, 2.12 mmol) at room temperature. The resulting mixture was degassed 5 min, the mixture was stirred at 100° C. for 18 h. After cooling to room temperature, the mixture was concentrated in vacuo. The residue purified by flash chromatography (silica gel, eluting with PE/EtOAc=100:0 to 0:100) to obtain 3-methylpyrido[2,3-d]pyridazin-5(6H)-one 1505 (2.4 g, 85% purity, 59% yield) as white solid.

LCMS (ESI) calcd for C₈H₇N₃O [M+H]⁺ m/z 162.07, found 162.

### Preparation of 3-methyl-2,3,4,6-tetrahydropyrido[2,3-d]pyridazin-5(1H)-one (1506)

To a solution of 3-methylpyrido[2,3-d]pyridazin-5(6H)-one 1505 (1.2 g, 7.4 mmol) in TFA (100 mL) was added PtO₂ (170 mg, 0.74 mmol) at room temperature. The resulting mixture was stirred at room temperature for 18 h under H₂ atmosphere. After cooling to room temperature, the mixture was concentrated in vacuo to obtain crude 3-methyl-2,3,4,6-tetrahydropyrido[2,3-d]pyridazin-5(1H)-one as TFA salt 1506 (1.2 g, 80% purity, 67% yield) as white solid.

LCMS (ESI) calcd for C₈H₁₁N₃O [M+H]⁺ m/z 166.10, found 166.

### Preparation of 6-(4-methoxybenzyl)-3-methyl-2,3,4,6-tetrahydropyrido[2,3-d]pyridazin-5(1H)-one (1507)

To a solution of 3-methyl-2,3,4,6-tetrahydropyrido[2,3-d]pyridazin-5(1H)-one as TFA salt (1506) (2.1 g, 0.0075 mol) in DMSO (80 mL) were added PMBCl (2.35 g, 0.015 mol) and Cs₂CO₃ (9.77 g, 0.03 mol). The reaction mixture was stirred at 50° C. for 18 h. The reaction solution was quenched with water and extracted with EtOAc (3×100 mL). The organic phase was concentrated under reduced pressure. The residue was purified by silica gel column (eluting with PE/EtOAc=90:10 to 80:20) to obtain 6-(4-methoxybenzyl)-3-methyl-2,3,4,6-tetrahydropyrido[2,3-d]pyridazin-5(1H)-one 1507 (0.5 g, 95% purity, 22% yield) as white solid.

LCMS (ESI) calcd for C₁₆H₁₉N₃O₂ [M+H]⁺ m/z 286.15 found 286.

### Preparation of ethyl 2-(6-(4-methoxybenzyl)-3-methyl-5-oxo-3,4,5,6-tetrahydropyrido[2,3-d]pyridazin-1(2H)-yl)acetate (1508)

To a solution of 6-(4-methoxybenzyl)-3-methyl-2,3,4,6-tetrahydropyrido[2,3-d]pyridazin-5(1H)-one 1507 (500 mg, 1.75 mmol) in DMF (40 mL) at 0° C. was added t-BuOK (590 mg, 5.26 mmol) in DMF (10 mL). The reaction mixture was stirred at 0° C. for 10 min. Then added ethyl 2-bromoacetate (878 mg, 5.25 mmol) in DMF (25 mL) at 0° C. The reaction mixture was stirred at 0° C. for 15 min, and then warmed to rt and stirred at rt for 1 h. The reaction solution was quenched with water and extracted with EtOAc (3×50 mL). The organic phase was concentrated under reduced pressure. The residue was purified by silica gel column (eluting with PE/EtOAc=90:10 to 0:100) to obtain ethyl 2-(6-(4-methoxybenzyl)-3-methyl-5-oxo-3,4,5,6-tetrahydropyrido[2,3-d]pyridazin-1(2H)-yl)acetate 1508 (560 mg, 90% purity, 77% yield) as yellow oil.

LCMS (ESI) calcd for C₂₀H₂₅N₃O₄ [M+H]⁺ m/z 372.18 found 372.

### Preparation of 1-(2-hydroxyethyl)-6-(4-methoxybenzyl)-3-methyl-2,3,4,6-tetrahydropyrido[2,3-d]pyridazin-5(1H)-one (1509)

To a solution of ethyl 2-(6-(4-methoxybenzyl)-3-methyl-5-oxo-3,4,5,6-tetrahydropyrido[2,3-d]pyridazin-1(2H)-yl)acetate 1508 (500 mg, 1.34 mmol) in THE (30 mL) at 0° C. was added LiAlH₄ (102 mg, 2.69 mmol). The reaction mixture was stirred at 0° C. for 15 min. The reaction solution was quenched with water and filtered. The filtered liquid was extracted with EtOAc (3×20 mL). The organic phase was concentrated under reduced pressure. The residue was purified by silica gel column (eluting with DCM/MeOH=100:0 to 95:5) to obtain 1-(2-hydroxyethyl)-6-(4-methoxybenzyl)-3-methyl-2,3,4,6-tetrahydropyrido[2,3-d]pyridazin-5(1H)-one 1509 (250 mg, 95% purity, 53% yield) as white solid.

LCMS (ESI) calcd for C₁₈H₂₃N₃O₃ [M+H]⁺ m/z 330.17, found 330.

### Preparation of (E)-6-(4-methoxybenzyl)-3-methyl-1-(2-((3-oxo-3-(4-(5-(trifluoromethyl) pyrimidin-2-yl)piperazin-1-yl)prop-1-en-1-yl)oxy)ethyl)-2,3,4,6-tetrahydropyrido[2,3-d]pyridazin-5(1H)-one (1510)

To a solution of 1-(2-hydroxyethyl)-6-(4-methoxybenzyl)-3-methyl-2,3,4,6-tetrahydropyrido[2,3-d]pyridazin-5(1H)-onee 1509 (140 mg, 0.42 mmol) in DCM (10 mL) at rt were added 1-(4-(5-(trifluoromethyl)pyrimidin-2-yl)piperazin-1-yl)prop-2-yn-1-one (133 mg, 0.47 mmol) and P(n-Bu)₃ (43 mg, 0.21 mmol). The reaction mixture was stirred at rt for 3 h. The reaction solution was concentrated under reduced pressure. The residue was purified by prep-TLC (eluting with DCM/MeOH=95:5) to obtain (E)-6-(4-methoxybenzyl)-3-methyl-1-(2-((3-oxo-3-(4-(5-(trifluoromethyl)pyrimidin-2-yl)piperazin-1-yl)prop-1-en-1-yl)oxy)ethyl)-2,3,4,6-tetrahydropyrido[2,3-d]pyridazin-5(1H)-one 1510 (310 mg, 60% purity, 71% yield) as white solid.

LCMS (ESI) calcd for C₃₀H₃₄F₃N₇O₄[M+H]⁺ m/z 614.26, found 614.35.

### Preparation of 6-(4-methoxybenzyl)-3-methyl-1-(2-(3-oxo-3-(4-(5-(trifluoromethyl)pyrimidin-2-yl)piperazin-1-yl)propoxy)ethyl)-2,3,4,6-tetrahydropyrido[2,3-d]pyridazin-5(1H)-one (1511)

To a solution of (E)-6-(4-methoxybenzyl)-3-methyl-1-(2-((3-oxo-3-(4-(5-(trifluoromethyl) pyrimidin-2-yl)piperazin-1-yl)prop-1-en-1-yl)oxy)ethyl)-2,3,4,6-tetrahydropyrido[2,3-d]pyridazin-5(1H)-one 1510 (310 mg, 0.50 mmol) in MeOH (10 mL) at rt was added Pd/C (32 mg, 0.30 mmol). The reaction mixture was stirred at rt for 18 h. The reaction solution was filtered and concentrated under reduced pressure to obtain 6-(4-methoxybenzyl)-3-methyl-1-(2-(3-oxo-3-(4-(5-(trifluoromethyl)pyrimidin-2-yl)piperazin-1-yl)propoxy)ethyl)-2,3,4,6-tetrahydropyrido[2,3-d]pyridazin-5(1H)-one 1511 (200 mg, 80% purity, 50% yield) as yellow oil.

LCMS (ESI) calcd for C₃₀H₃₆F₃N₇O₄[M+H]+m/z 616.28, found 616.

### Preparation of 3-methyl-1-(2-(3-oxo-3-(4-(5-(trifluoromethyl)pyrimidin-2-yl)piperazin-1-yl)propoxy)ethyl)-2,3,4,6-tetrahydropyrido[2,3-d]pyridazin-5(1H)-one (Mixture of 64 and 65)

To a solution of 6-(4-methoxybenzyl)-3-methyl-1-(2-(3-oxo-3-(4-(5-(trifluoromethyl) pyrimidin-2-yl)piperazin-1-yl)propoxy)ethyl)-2,3,4,6-tetrahydropyrido[2,3-d]pyridazin-5(1i)-one 1511 (200 mg, 0.32 mmol) in TFA (20 mL) was stirred at 100° C. overnight. The solvent was removed under reduced pressure. Then the reaction was quenched with water and adjusted pH to 7˜8 with 1 M NaOH aqueous. The aqueous phase was purified by C18 column (Agela 40 g, mobile phase: ACN-H₂O (0.1% FA), gradient: 50-95) to give 3-methyl-1-(2-(3-oxo-3-(4-(5-(trifluoromethyl) pyrimidin-2-yl) piperazin-1-yl) propoxy) ethyl)-2,3,4,6-tetrahydropyrido[2,3-d]pyridazin-5(1H)-one (mixture of 64+65) (40 mg, 97% purity, 24% yield) as white solid.

LCMS (ESI) calcd for C₂₂H₂₈F₃N₇O₃[M+H]+m/z 496.22, found 496.

Chiral resolution of 3-methyl-1-(2-(3-oxo-3-(4-(5-(trifluoromethyl)pyrimidin-2-yl)piperazin-1-yl)propoxy)ethyl)-2,3,4,6-tetrahydropyrido[2,3-d]pyridazin-5(1H)-one (64 and 65) Compound 64+65 was separated by SFC (Column: Daicel CHIRALPAK OJ-H 250 mm×20 mm I.D., 5 mm; Mobile phase: CO₂/MeOH [0.2% (NH₃)]=70/30) and concentrated under reduced pressure to afford the first fraction as 64 (20.5 mg, 99% purity, ee %: 100, white solid) and the second fraction as 65 (16.1 mg, 100% purity, ee %: 99, white solid) Although two fractions were obtained it was not possible from this information to assign absolute stereochemistry to each enantiomer.

### Compound 64

¹H NMR (400 MHz, DMSO-d₆, ppm) δ:12.02 (s, 1H), 8.73 (d, J=0.8 Hz, 2H), 7.72 (s, 1H), 3.87-3.76 (m, 4H), 3.65 (t, J=6.4 Hz, 2H), 3.61-3.44 (m, 8H), 3.26-3.22 (m, 1H), 2.94-2.86 (m, 1H), 2.57 (t, J=6.4 Hz, 2H), 2.54-2.52 (m, 1H), 1.90-1.79 (m, 2H), 0.95 (d, J=6.4 Hz, 3H).

LCMS (ESI) calcd for C₂₂H₂₈F₃N₇O₃[M+H]+m/z 496.22, found 496.

### Compound 65

¹H NMR (400 MHz, DMSO-d₆, ppm) δ: 12.02 (s, 1H), 8.73 (d, J=0.8 Hz, 2H), 7.72 (s, 1H), 3.87-3.76 (m, 4H), 3.66 (t, J=6.4 Hz, 2H), 3.60-3.45 (m, 8H), 3.27-3.21 (m, 1H), 2.95-2.87 (m, 1H), 2.57 (t, J=6.6 Hz, 2H), 2.54-2.51 (m, 1H), 1.91-1.77 (m, 2H), 0.95 (d, J=6.4 Hz, 3H).

LCMS (ESI) calcd for C₂₂H₂₈F₃N₇O₃[M+H]+m/z 496.22, found 496.

### 7. Synthesis of 5-(1-(3-oxo-3-(4-(5-(trifluoromethyl)pyrimidin-2-yl)piperazin-1-yl) propoxy) propan-2-yl)phthalazin-1(2H)-one (Compounds 72 and 73)

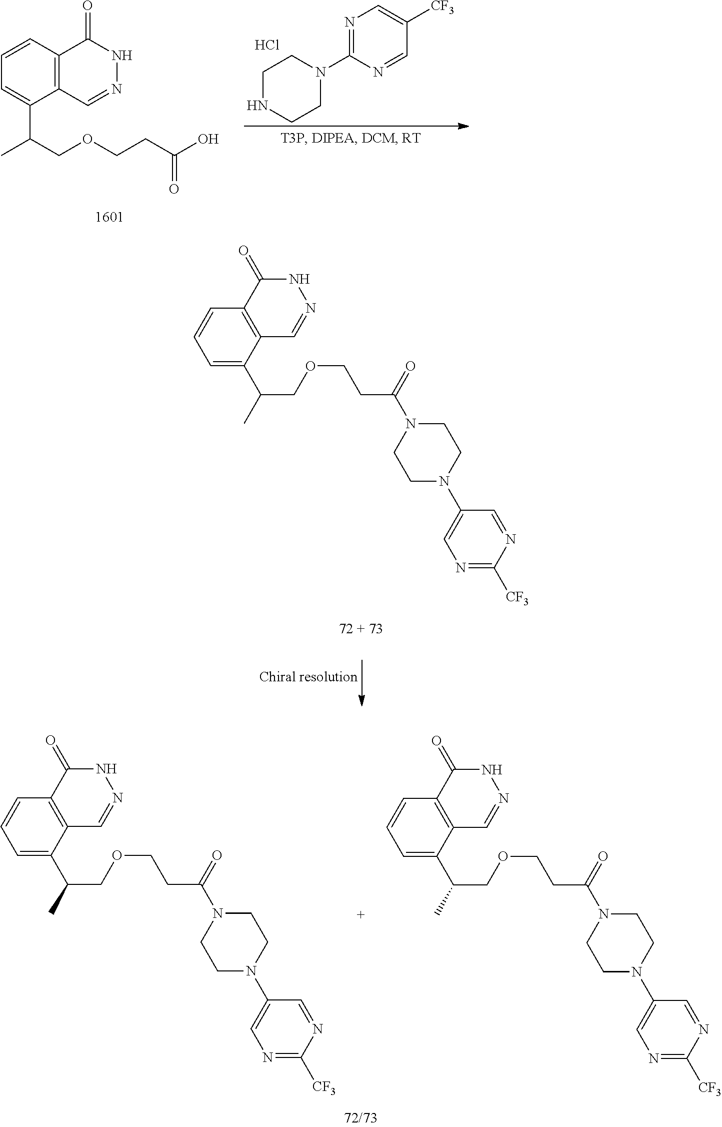 [SMILES: CC(COCCC(=O)O)c1cccc2c(=O)[nH]ncc12 ; Cl ; FC(F)(F)c1cnc(N2CCNCC2)nc1 ; CC(COCCC(=O)N1CCN(c2cnc(C(F)(F)F)nc2)CC1)c1cccc2c(=O)[nH]ncc12 ; C[C@H](COCCC(=O)N1CCN(c2cnc(C(F)(F)F)nc2)CC1)c1cccc2c(=O)[nH]ncc12 ; C[C@@H](COCCC(=O)N1CCN(c2cnc(C(F)(F)F)nc2)CC1)c1cccc2c(=O)[nH]ncc12]

### Preparation of 5-(1-(3-oxo-3-(4-(5-(trifluoromethyl)pyrimidin-2-yl)piperazin-1-yl)propoxy) propan-2-yl)phthalazin-1(2H)-one (Mixture of 72 and 73)

To a solution of 3-(2-(1-oxo-1,2-dihydrophthalazin-5-yl)propoxy)propanoic acid 1601 (150 mg, 0.543 mmol) in DCM (15 mL) were added 2-(piperazin-1-yl)-5-(trifluoromethyl)pyrimidine hydrochloride (220 mg, 0.814 mmol), DIPEA (351 mg, 2.715 mmol), T3P (50% in EtOAc, 518 mg, 0.814 mmol) at room temperature successively. The mixture was kept stirring at room temperature for 1 h. The resulting mixture was diluted with water and extracted with DCM (30 mL×3). The combined organic layer was dried over Na₂SO₄ and concentrated under reduced pressure. The residue was purified by C18 column (Agela 40 g, mobile phase: ACN-H₂O (0.1% FA), gradient: 40-60) to give 5-(1-(3-oxo-3-(4-(5-(trifluoromethyl)pyrimidin-2-yl)piperazin-1-yl)propoxy) propan-2-yl)phthalazin-1(2H)-one (mixture of 72 and 73) (80 mg, 99% purity, 30% yield) as a white solid.

LCMS (ESI) calcd for C₂₃H₂₅F₃N₆O₃[M+H]⁺ m/z 491.20, found 491.

### Chiral resolution of 5-(1-(3-oxo-3-(4-(5-(trifluoromethyl)pyrimidin-2-yl)piperazin-1-yl) propoxy) propan-2-yl)phthalazin-1(2H)-one (72 and 73)

Compound 72+73 was separated by SFC (Column: Daicel CHIRALPAK AS-H 250 mm×20 mm I.D., 5 mm; Mobile phase: CO₂/MeOH [0.1% (NH₃)]=70/30) and concentrated under reduced pressure to afford the first fraction as 72 (34.9 mg, 100% purity, ee %: 100, white solid) and the second fraction as 73 (37.2 mg, 99% purity, ee %: 100, white solid)

### Compound 72

¹H NMR (400 MHz, DMSO-d₆, ppm) δ: 12.64 (s, 1H), 8.72 (d, J=0.8 Hz, 2H), 8.58 (s, 1H), 8.09 (d, J=7.6 Hz, 1H), 7.87-7.82 (m, 1H), 7.80-7.75 (m, 1H), 3.84-3.61 (m, 9H), 3.52-3.44 (m, 4H), 2.56-2.53 (m, 2H), 1.26 (d, J=6.8 Hz, 3H).

LCMS (ESI) calcd for C₂₃H₂₅F₃N₆O₃[M+H]⁺ m/z 491.20, found 491.

### Compound 73

¹H NMR (400 MHz, DMSO-d₆, ppm) δ: 12.64 (s, 1H), 8.72 (d, J=0.7 Hz, 2H), 8.58 (s, 1H), 8.09 (d, J=7.6 Hz, 1H), 7.88-7.83 (m, 1H), 7.80-7.75 (m, 1H), 3.83-3.61 (m, 9H), 3.51-3.43 (m, 4H), 2.56-2.52 (m, 2H), 1.26 (d, J=6.8 Hz, 3H).

LCMS (ESI) calcd for C₂₃H₂₅F₃N₆O₃[M+H]⁺ m/z 491.20, found 491.

### 8. Synthesis of 3-methyl-1-(2-(3-oxo-3-(4-(5-(trifluoromethyl)pyrimidin-2-yl)piperazin-1-yl)propoxy)ethyl)-1,5-dihydro-4H-pyrrolo[2,3-d]pyridazin-4-one (Compound 74)

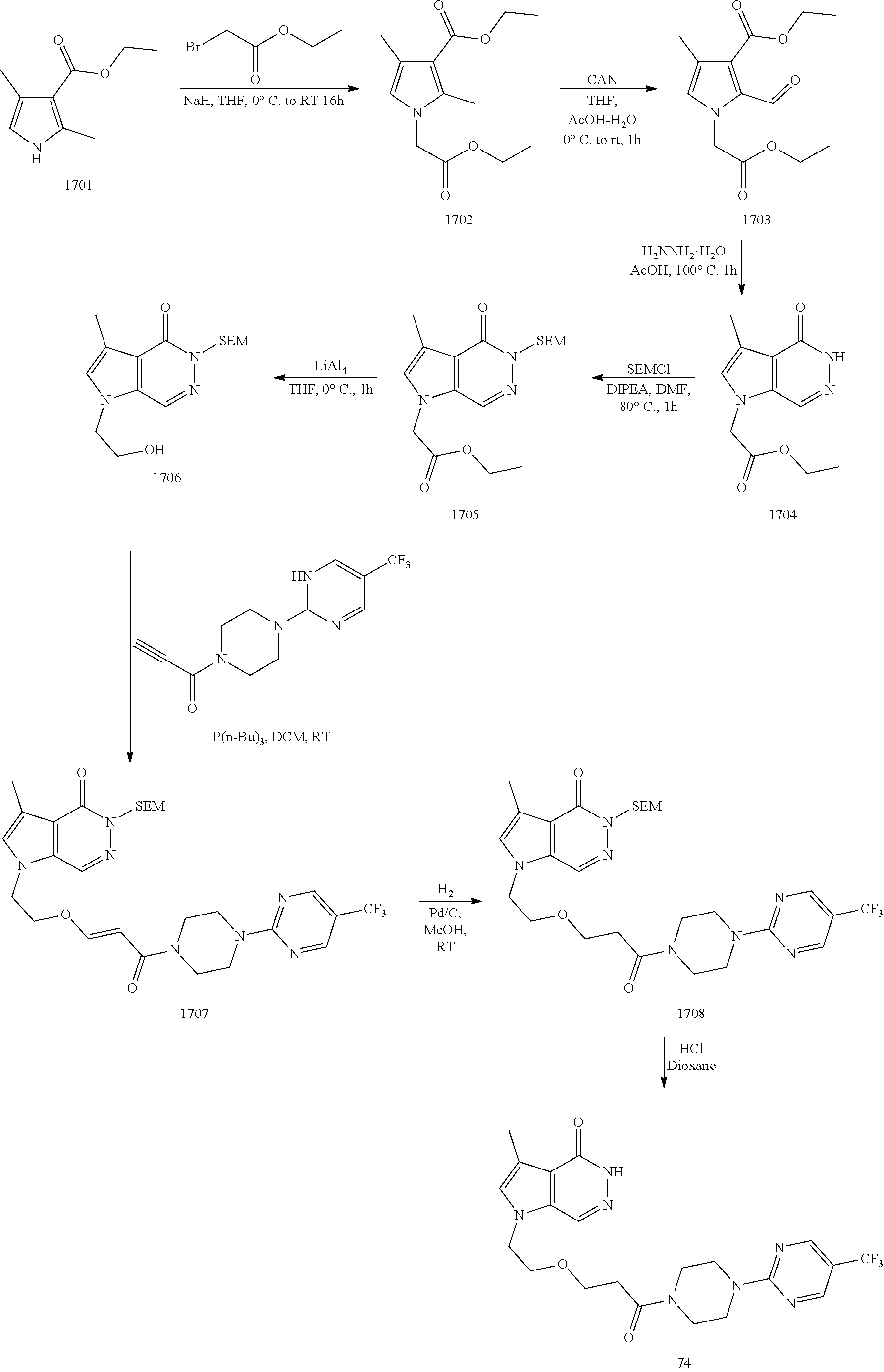

### Preparation of ethyl 1-(2-ethoxy-2-oxoethyl)-2,4-dimethyl-1H-pyrrole-3-carboxylate (1702)

To a stirred solution of NaH (60% in oil, 3.2 g, 80.0 mmol) in THF (100 mL) at 0° C. was added ethyl 2,4-dimethyl-1H-pyrrole-3-carboxylate 1701 (2 g, 11.8 mmol) in portions. After stirring at 0° C. for 15 min, ethyl 2-bromoacetate (10.1 g, 60.9 mmol) was added dropwise and the reaction was warmed to rt and stirred for 16 h. The reaction was quenched with saturated aqueous NH₄Cl and extracted with EtOAc (50 mL×4). The combined organic layers were washed with brine (30 mL), dried over Na₂SO₄ and concentrated in vacuo. The residue was purified by flash chromatography (eluting with PE/EtOAc=100:0 to 95:5) to afford ethyl 1-(2-ethoxy-2-oxoethyl)-2,4-dimethyl-1H-pyrrole-3-carboxylate 1702 (2.63 g, 98% purity, 72% yield) as an off-white solid.

LCMS (ESI) calcd for C₁₃H₁₉NO₄ [M+H]⁺ m/z 254.13, found 254.10.

### Preparation of ethyl 1-(2-ethoxy-2-oxoethyl)-2-formyl-4-methyl-1H-pyrrole-3-carboxylate (1703)

Ethyl 1-(2-ethoxy-2-oxoethyl)-2,4-dimethyl-1H-pyrrole-3-carboxylate 1702 (2.63 g, 10.38 mmol) was dissolved in THE (120 mL) under stirring, followed by addition a solution of AcOH (120 mL) and H₂O (120 mL). The mixture was homogeneously stirred at 0° C. and added with CAN (33.66 g, 61.54 mol) in one portion. After stirring at rt for 1 h, the reaction mixture was poured into ice-water (120 mL) and stirred for another 30 min. The resulting solution was extracted with EtOAc (20 mL×3). The combined organic layer was washed with brine, dried over Na₂SO₄ and concentrated under reduced pressure. The residue was purified by flash chromatography (eluting with PE/EtOAc=100:0 to 90:10) to obtain the title compound ethyl 1-(2-ethoxy-2-oxoethyl)-2-formyl-4-methyl-1H-pyrrole-3-carboxylate 1703 (1.20 g, 98% purity, 46% yield) as a white solid.

### Preparation of ethyl 2-(3-methyl-4-oxo-4,5-dihydro-1H-pyrrolo[2,3-d]pyridazin-1-yl)acetate (1704)

To a solution of ethyl 1-(2-ethoxy-2-oxoethyl)-2-formyl-4-methyl-1H-pyrrole-3-carboxylate 1703 (1.20 g, 4.47 mmol) in AcOH (5 mL) was added H₂NNH₂·H₂O (80% wt, 0.46 g, 6.73 mmol) in one portion. The reaction mixture was heated with stirring at 100° C. for 1 h. Most of the solvent was removed by evaporation (55° C.) under reduced pressure. The resulting solution was extracted with EtOAc (20 mL×3). The combined organic layer was washed with brine, dried over Na₂SO₄ and concentrated under reduced pressure. The residue was purified by flash chromatography (eluting with PE/EtOAc=100:0 to 50:50) to obtain the title compound ethyl 2-(3-methyl-4-oxo-4,5-dihydro-1H-pyrrolo[2,3-d]pyridazin-1-yl)acetate 1704 (603 mg, 97% purity, 47% yield) as a white solid.

LCMS (ESI) calcd for C₁₁H₁₃N₃O₃ [M+H]⁺ m/z 236.10, found 236.

### Preparation of ethyl 2-(3-methyl-4-oxo-5-((2-(trimethylsilyl)ethoxy)methyl)-4,5-dihydro-1H-pyrrolo[2,3-d]pyridazin-1-yl)acetate (1705)

To a solution ethyl 2-(3-methyl-4-oxo-4,5-dihydro-1H-pyrrolo[2,3-d]pyridazin-1-yl)acetate 1704 (603 mg, 2.58 mmol) and DIPEA (1664 mg, 12.8 mmol) in DMF (42 mL) at rt, SEMCl (2127 mg, 12.8 mmol) was added in one portion. After completing the addition, the reaction solution was heated at 80° C. for 1 h. The resulting reaction solution was poured into cold water and then extracted with EtOAc (20 mL×3). The combined organic layer was washed with brine, dried over Na₂SO₄ and concentrated under reduced pressure. The residue was purified by flash chromatography (eluting with PE/EtOAc=100:0 to 60:40) to give ethyl 2-(3-methyl-4-oxo-5-((2-(trimethylsilyl)ethoxy)methyl)-4,5-dihydro-1H-pyrrolo[2,3-d]pyridazin-1-yl)acetate 1705 (565 mg, 98% purity, 82% yield) as a white solid.

LCMS (ESI) calcd for C₁₇H₂₇N₃O₄Si [M+H]⁺ m/z 366.18, found 366.15.

### Preparation of 1-(2-hydroxyethyl)-3-methyl-5-((2-(trimethylsilyl)ethoxy)methyl)-1,5-dihydro-4H-pyrrolo[2,3-d]pyridazin-4-one (1706)

To a suspension of LiAlH₄ (55 mg, 1.449 mmol) in THE (5 mL), ethyl 2-(3-methyl-4-oxo-5-((2-(trimethylsilyl)ethoxy)methyl)-4,5-dihydro-1H-pyrrolo[2,3-d]pyridazin-1-yl)acetate 1705 (350 mg, 0.956 mmol) in THE (5 mL) was added dropwise under N₂ atmosphere, in which the temperature was kept at 0˜5° C. The reaction mixture was stirred at this temperature for a further 1 h. After the dropwise addition of water (0.1 mL) and 13% aq. NaOH (0.2 mL) successively, the mixture was stirred for additional 30 min. The resulting mixture was filtered through diatomaceous earth and the filter cake was thoroughly washed with DCM. The filtrate was concentrated to dryness under reduced pressure. The residue was purified by flash chromatography (eluting with DCM/MeOH=100:0 to 90:10) to obtain 1-(2-hydroxyethyl)-3-methyl-5-((2-(trimethylsilyl)ethoxy)methyl)-1,5-dihydro-4H-pyrrolo[2,3-d]pyridazin-4-one 1706 (280 mg, 98% purity, 75% yield) as a yellow oil.

LCMS (ESI) calcd for C₁₅H₂₅N₃O₃Si [M+H]⁺ m/z 324.17, found 324.15.

### Preparation of (E)-3-methyl-1-(2-((3-oxo-3-(4-(5-(trifluoromethyl)pyrimidin-2-yl)piperazin-1-yl)prop-1-en-1-yl)oxy)ethyl)-5-((2-(trimethylsilyl)ethoxy)methyl)-1,5-dihydro-4H-pyrrolo[2,3-d]pyridazin-4-one (1707)

A round-bottom flask containing a mixture of 1-(4-(5-(trifluoromethyl)pyrimidin-2-yl)piperazin-1-yl)prop-2-yn-1-one (80 mg, 0.282 mmol), 1-(2-hydroxyethyl)-3-methyl-5-((2-(trimethylsilyl)ethoxy)methyl)-1,5-dihydro-4H-pyrrolo[2,3-d]pyridazin-4-one 1706 (90 mg, 0.279 mmol) and P(n-Bu)₃ (49 mg, 0.241 mmol) in the DCM (9 mL) was placed in oil bath heated to 25° C. and stirred under 25° C. of 18 h. The resulting reaction solution was poured into cold water and then extracted with DCM (100 mL×3). The combined organic layer was washed with brine, dried over Na₂SO₄ and concentrated under reduced pressure. The residue was purified by flash chromatography (eluting with DCM/MeOH=100:0 to 90:10) to give of (E)-3-methyl-1-(2-((3-oxo-3-(4-(5-(trifluoromethyl)pyrimidin-2-yl)piperazin-1-yl)prop-1-en-1-yl)oxy)ethyl)-5-((2-(trimethylsilyl)ethoxy)methyl)-1,5-dihydro-4H-pyrrolo[2,3-d]pyridazin-4-one 1707 (172 mg, 98% purity, 79% yield) as a yellow solid.

LCMS (ESI) calcd for C₂₇H₃₆F₃N₇O₄Si [M+H]⁺ m/z 608.26, found 608.25.

### Preparation of 3-methyl-1-(2-(3-oxo-3-(4-(5-(trifluoromethyl)pyrimidin-2-yl)piperazin-1-yl)propoxy)ethyl)-5-((2-(trimethylsilyl)ethoxy)methyl)-1,5-dihydro-4H-pyrrolo[2,3-d]pyridazin-4-one (1708)

A mixture of compound (E)-3-methyl-1-(2-((3-oxo-3-(4-(5-(trifluoromethyl)pyrimidin-2-yl)piperazin-1-yl)prop-1-en-1-yl)oxy)ethyl)-5-((2-(trimethylsilyl)ethoxy)methyl)-1,5-dihydro-4H-pyrrolo[2,3-d]pyridazin-4-one 1707 (152 mg, 0.250 mmol) and Pd/C (31 mg) in MeOH (20 mL) was stirred under 1 atm at room temperature for 6 hour. The resulting mixture was filtered through diatomaceous earth. The filtrate was concentrated to dryness under reduced pressure. The residue was purified by flash chromatography (eluting with DCM/MeOH=100:0 to 98:2) to 3-methyl-1-(2-(3-oxo-3-(4-(5-(trifluoromethyl)pyrimidin-2-yl)piperazin-1-yl)propoxy)ethyl)-5-((2-(trimethylsilyl)ethoxy)methyl)-1,5-dihydro-4H-pyrrolo[2,3-d]pyridazin-4-one 1708 (120 mg, 98% purity, 78% yield) as a yellow solid.

LCMS (ESI) calcd for C₂₇H₃₈F₃N₇O₄Si [M+H]⁺ m/z 610.27, found 610.30.

### Preparation of 3-methyl-1-(2-(3-oxo-3-(4-(5-(trifluoromethyl)pyrimidin-2-yl)piperazin-1-yl)propoxy)ethyl)-1,5-dihydro-4H-pyrrolo[2,3-d]pyridazin-4-one (Compound 74)

A round-bottom flask containing a mixture of 3-methyl-1-(2-(3-oxo-3-(4-(5-(trifluoromethyl)pyrimidin-2-yl)piperazin-1-yl)propoxy)ethyl)-5((2(trimethylsilyl)ethoxy)methyl)-1,5-dihydro-4H-pyrrolo[2,3-d]pyridazin-4-one 1708 (120-mg, 0.197 mmol) and HCl-Dioxane (4 M, 10 mL) was stirred at rt for 2 h. The resulting mixture was concentrated to dryness under reduced pressure. The residue was purified by C18 column (Agela 40 g, mobile phase: ACN-H₂O (0.1% FA), gradient: 30-70) to to 3-methyl-1-(2-(3-oxo-3-(4-(5-(trifluoromethyl)pyrimidin-2-yl)piperazin-1-yl)propoxy)ethyl)-1,5-dihydro-4H-pyrrolo[2,3-d]pyridazin-4-one 74 (40 mg, 99% purity, 40% yield) as a white solid.

¹H NMR (400 MHz, DMSO-d₆, ppm) δ:12.04 (s, 1H), 8.73 (d, J=0.8 Hz, 2H), 8.24 (s, 1H), 7.14 (d, J=0.8 Hz, 1H), 4.29 (t, J=5.0 Hz, 2H), 3.84-3.74 (m, 4H), 3.69 (t, J=5.0 Hz, 2H), 3.63 (t, J=6.4 Hz, 2H), 3.56-3.46 (m, 4H), 2.56-2.51 (m, 2H), 2.30 (s, 3H).

LCMS (ESI) calcd for C₂₁H₂₄F₃N₇O₃[M+H]⁺ m/z 480.19, found 480.25.

### 9. Synthesis of 1-(2-(3-oxo-3-(4-(5-(trifluoromethyl)pyrimidin-2-yl)piperazin-1-yl)propoxy)ethyl)-3-(trifluoromethyl)-1,5-dihydro-4H-pyrrolo[2,3-d]pyridazin-4-one (Compound 131)

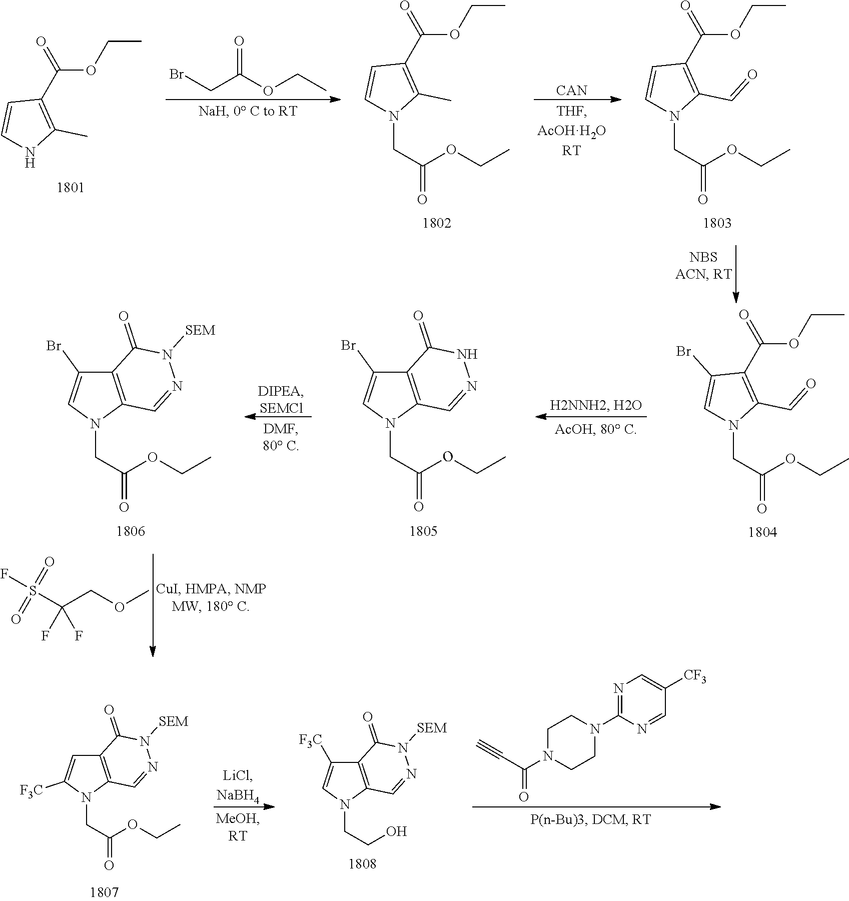

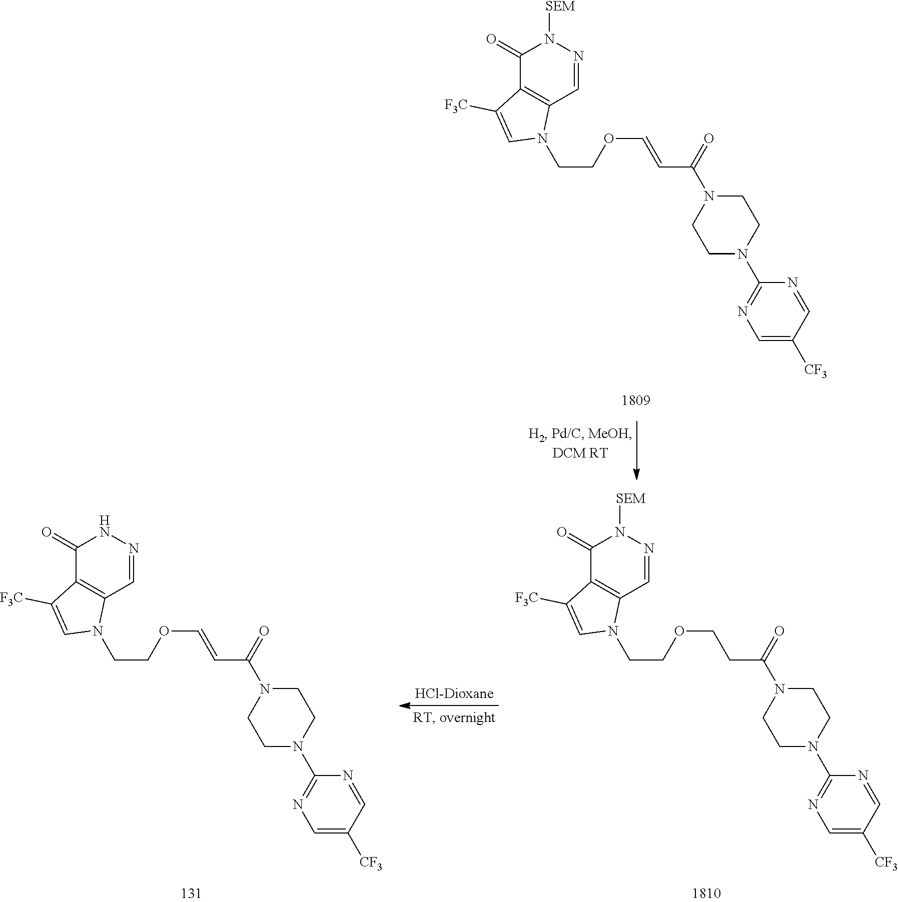 [SMILES: C[Si](C)(C)CCOCn1ncc2c(c(C(F)(F)F)cn2CCO/C=C/C(=O)N2CCN(c3ncc(C(F)(F)F)cn3)CC2)c1=O ; O=C(/C=C/OCCn1cc(C(F)(F)F)c2c(=O)[nH]ncc21)N1CCN(c2ncc(C(F)(F)F)cn2)CC1 ; C[Si](C)(C)CCOCn1ncc2c(c(C(F)(F)F)cn2CCOCCC(=O)N2CCN(c3ncc(C(F)(F)F)cn3)CC2)c1=O]

### Preparation of ethyl 1-(2-ethoxy-2-oxoethyl)-2-methyl-1H-pyrrole-3-carboxylate (1802)

To a solution of ethyl 2-methyl-1H-pyrrole-3-carboxylate 1801 (10 g, 0.065 mol) in THE (100 mL) were added ethyl 2-bromoacetate (16.3 g, 0.097 mol), NaH (60% in oil, 3.9 g, 0.097 mol) at 0° C. After completion of addition, the reaction solution was warmed to rt and kept stirring at rt for an additional 16 h. Water was added to quench the reaction. The obtained solution was extracted with EtOAc (50 mL×4). The combined organic phase was washed with brine, dried over Na₂SO₄ and concentrated under reduced pressure. The residue was purified by flash chromatography (eluting with PE/EtOAc=100:0 to 50:50) to give ethyl 1-(2-ethoxy-2-oxoethyl)-2-methyl-1H-pyrrole-3-carboxylate 1802 (14 g, 90% purity, 80% yield) as yellow solid.

LCMS (ESI) calcd for C₁₂H₁₇NO₄ [M+H]⁺ m/z 240.12, found 240.

### Preparation of ethyl 1-(2-ethoxy-2-oxoethyl)-2-formyl-1H-pyrrole-3-carboxylate (1803)

To a stirred solution of ethyl 1-(2-ethoxy-2-oxoethyl)-2-methyl-1H-pyrrole-3-carboxylate 1802 (10 g, 0.041 mol) in THF: CH₃COOH:H₂O (1:1:1, 150 mL) was added CAN (91.6 g, 0.167 mol) at rt. The reaction mixture was stirred at room temperature for 6 h. The reaction mixture was poured carefully into an ice bath-cooled solution, and then extracted with EtOAc (2×500 mL). The combined extracts were washed with NaHCO₃ solution (500 mL). The organic layer dried over sodium sulfate and concentrated under reduced pressure to give crude. The crude was purified by flash column chromatography (eluting with PE/EtOAc=99:1 to 50:50) to afford ethyl 1-(2-ethoxy-2-oxoethyl)-2-formyl-1H-pyrrole-3-carboxylate 1803 (6 g, 90% purity, 50% yield) as yellow oil.

LCMS (ESI) calcd for C₁₂H₁₅NO₅ [M+H]⁺ m/z 254.10, found 254.

### Preparation of ethyl 4-bromo-1-(2-ethoxy-2-oxoethyl)-2-formyl-1H-pyrrole-3-carboxylate (1804)

To the solution of ethyl 1-(2-ethoxy-2-oxoethyl)-2-formyl-1H-pyrrole-3-carboxylate 1803 (3.0 g, 0.011 mol) in MeCN (50 mL) was added NBS (1.0 g, 0.005 mmol) at rt. The mixture was kept stirring at room temperature for 1 h. The resulting mixture was diluted with water and extracted with DCM (30 mL×3). The combined organic layer was dried over Na₂SO₄ and concentrated under reduced pressure. The residue was purified by flash chromatography (eluting with PE/EtOAc=100:0 to 70:30) to give ethyl 4-bromo-1-(2-ethoxy-2-oxoethyl)-2-formyl-1H-pyrrole-3-carboxylate 1804 (1 g, 90% purity, 22% yield) as yellow oil.

LCMS (ESI) calcd for C₁₂H₁₄BrNO₅ [M+H]⁺ m/z 332.01, found 332, 334.

### Preparation of ethyl 2-(3-bromo-4-oxo-4,5-dihydro-1H-pyrrolo[2,3-d]pyridazin-1-yl)acetate (1805)

To a solution of ethyl 4-bromo-1-(2-ethoxy-2-oxoethyl)-2-formyl-1H-pyrrole-3-carboxylate 1804 (1.2 g, 0.003 mol) in AcOH (20 mL) was added H₂NNH₂·H₂O (80% wt, 0.54 g, 0.010 mol) at rt. The mixture was kept stirring at 80° C. for 1 h. The resulting light brown solution was concentrated under reduced pressure to remove most AcOH. The residue was diluted with DCM (50 mL) and then adjusted PH to 8 with saturated aqueous NaHCO₃ at 0° C. The basified solution was extracted with DCM (10 mL×3). The combined organic layer was washed with brine, dried over Na₂SO₄ and concentrated under reduced pressure to give ethyl 2-(3-bromo-4-oxo-4,5-dihydro-1H-pyrrolo[2,3-d]pyridazin-1-yl)acetate 1805 (1.1 g, 90% purity, 91% yield) as yellow solid.

LCMS (ESI) calcd for C₁₁₀H₁₀BrN₃O₃[M+H]⁺ m/z 299.99, found 300, 302.

### Preparation of ethyl 2-(3-bromo-4-oxo-5-((2-(trimethylsilyl)ethoxy)methyl)-4,5-dihydro-1H-pyrrolo[2,3-d]pyridazin-1-yl)acetate (1806)

To a solution of ethyl 2-(3-bromo-4-oxo-4,5-dihydro-1H-pyrrolo[2,3-d]pyridazin-1-yl)acetate 1805 (2 g, 0.006 mol), DIPEA (2.6 g, 0.020 mol) in DMF (30 mL) was added SEMCl (2.2 g, 0.013 mol) at rt. Then the reaction mixture was stirred 80° C. for 1 h. After cooling to room temperature, the reaction mixture was poured into cold water and then extracted with EtOAc (50 mL×4). The combined organic layer was washed three times with brine solution, dried over Na₂SO₄ and concentrated under reduced pressure. The residue was purified by flash chromatography (eluting with PE/EtOAc=100:0 to 40:60) to afford ethyl 2-(3-bromo-4-oxo-5-((2-(trimethylsilyl)ethoxy)methyl)-4,5-dihydro-1H-pyrrolo[2,3-d]pyridazin-1-yl)acetate 1806 (2 g, 90% purity, 62% yield) as yellow oil.

LCMS (ESI) calcd for C₁₆H₂₄BrN₃O₄Si [M+H]⁺ m/z 430.07, found 430, 432.

### Preparation of ethyl 2-(4-oxo-3-(trifluoromethyl)-5-((2-(trimethylsilyl)ethoxy)methyl)-4,5-dihydro-1H-pyrrolo[2,3-d]pyridazin-1-yl)acetate (1807)

To a stirred solution of ethyl 2-(3-bromo-4-oxo-5-((2-(trimethylsilyl)ethoxy)methyl)-4,5-dihydro-1H-pyrrolo[2,3-d]pyridazin-1-yl)acetate 1806 (300 mg, 0.69 mmol), CuI (131 mg, 0.69 mmol), HMPA (621 mg, 3.45 mmol) in NMP (10 mL) was added methyl 2,2-difluoro-2-(fluorosulfonyl)acetate (662 mg, 3.45 mmol) at rt. The solution was then stirred in a microwave at 160° C. for 1 h. The resulting reaction solution was filtered and the filtrate was purified directly by flash silica chromatography (eluting with PE/EtOAc=100:0 to 50:50) to give ethyl 2-(4-oxo-3-(trifluoromethyl)-5-((2-(trimethylsilyl)ethoxy)methyl)-4,5-dihydro-1H-pyrrolo[2,3-d]pyridazin-1-yl)acetate 1807 (150 mg, 30% purity, 12% yield) as yellow oil.

LCMS (ESI) calcd for C₁₇H₂₄F₃N₃O₄Si [M+H]⁺ m/z 420.15, found 420.

### Preparation of 1-(2-hydroxyethyl)-3-(trifluoromethyl)-5-((2-(trimethylsilyl)ethoxy)methyl)-1,5-dihydro-4H-pyrrolo[2,3-d]pyridazin-4-one (1808)

To a solution of ethyl 2-(4-oxo-3-(trifluoromethyl)-5-((2-(trimethylsilyl)ethoxy)methyl)-4,5-dihydro-1H-pyrrolo[2,3-d]pyridazin-1-yl)acetate 1807 (900 mg, 2.13 mmol) in MeOH (20 mL) were added LiCl (362 mg, 8.54 mmol), NaBH₄ (323 mg, 8.54 mmol) at rt successively. The reaction mixture was stirred at rt for 1 h. The resulting mixture was quenched with water and extracted with DCM (50 mL×3). The combined organic layer was washed with brine and concentrated under reduced pressure. The residue was purified by silica gel column (eluting with PE/EtOAc=100:0 to 50:50) to obtain 1-(2-hydroxyethyl)-3-(trifluoromethyl)-5-((2-(trimethylsilyl)ethoxy)methyl)-1,5-dihydro-4H-pyrrolo[2,3-d]pyridazin-4-one 1808 (150 mg, 90% purity, 16% yield) as colorless oil.

LCMS (ESI) calcd for C₁₅H₂₂F₃N₃O₃Si [M+H]⁺ m/z 378.14, found 378.15.

### Preparation of (E)-1-(2-((3-oxo-3-(4-(5-(trifluoromethyl)pyrimidin-2-yl)piperazin-1-yl)prop-1-en-1-yl)oxy)ethyl)-3-(trifluoromethyl)-5-((2-(trimethylsilyl)ethoxy)methyl)-1,5-dihydro-4H-pyrrolo[2,3-d]pyridazin-4-one (1809)

To a solution of 1-(2-hydroxyethyl)-3-(trifluoromethyl)-5-((2-(trimethylsilyl)ethoxy)methyl)-1,5-dihydro-4H-pyrrolo[2,3-d]pyridazin-4-one 1808 (100 mg, 0.26 mmol), 1-(4-(5-(trifluoromethyl)pyrimidin-2-yl)piperazin-1-yl)prop-2-yn-1-one (112 mg, 0.39 mmol) in DCM (10 mL) was added P(n-Bu)₃ (5 mg, 0.026 mmol) at rt. The reaction mixture was stirred at rt for 1 h. The resulting mixture was quenched with water and extracted with DCM (50 mL×3). The combined organic layer was washed with brine and concentrated under reduced pressure. The residue was purified by silica gel column (eluting with PE/EtOAc=100:0 to 0:100) to obtain (E)-1-(2-((3-oxo-3-(4-(5-(trifluoromethyl)pyrimidin-2-yl)piperazin-1-yl)prop-1-en-1-yl)oxy)ethyl)-3-(trifluoromethyl)-5-((2-(trimethylsilyl)ethoxy)methyl)-1,5-dihydro-4H-pyrrolo[2,3-d]pyridazin-4-one 1809 (180 mg, 90% purity, 92% yield) as colorless oil.

LCMS (ESI) calcd for C₂₇H₃₃F₆N₇O₄Si [M+H]⁺ m/z 662.23, found 662.25.

### Preparation of 1-(2-(3-oxo-3-(4-(5-(trifluoromethyl)pyrimidin-2-yl)piperazin-1-yl)propoxy)ethyl)-3-(trifluoromethyl)-5-((2-(trimethylsilyl)ethoxy)methyl)-1,5-dihydro-4H-pyrrolo[2,3-d]pyridazin-4-one (1810)

A solution of (E)-1-(2-((3-oxo-3-(4-(5-(trifluoromethyl)pyrimidin-2-yl)piperazin-1-yl)prop-1-en-1-yl)oxy)ethyl)-3-(trifluoromethyl)-5-((2-(trimethylsilyl)ethoxy)methyl)-1,5-dihydro-4H-pyrrolo[2,3-d]pyridazin-4-one 1809 (150 mg, 0.22 mmol) and Pd/C (30 mg) in MeOH/DCM (15 mL, 2:1) was stirred at rt for 1 h under H₂ atmosphere. The resulting solution was filtered through diatomaceous earth and the filter cake was washed with DCM (5 mL×4). The filtrate was concentrated under reduced pressure to give 1-(2-(3-oxo-3-(4-(5-(trifluoromethyl)pyrimidin-2-yl)piperazin-1-yl)propoxy)ethyl)-3-(trifluoromethyl)-5-((2-(trimethylsilyl)ethoxy)methyl)-1,5-dihydro-4H-pyrrolo[2,3-d]pyridazin-4-one 1810 (140 mg, 90% purity, 83% yield) as colorless oil.

LCMS (ESI) calcd for C₂₇H₃₅F₆N₇O₄Si [M+H]⁺ m/z 664.24, found 664.25.

### Preparation of 1-(2-(3-oxo-3-(4-(5-(trifluoromethyl)pyrimidin-2-yl)piperazin-1-yl)propoxy)ethyl)-3-(trifluoromethyl)-1,5-dihydro-4H-pyrrolo[2,3-d]pyridazin-4-one (Compound 131)

To a solution of 1-(2-(3-oxo-3-(4-(5-(trifluoromethyl)pyrimidin-2-yl)piperazin-1-yl)propoxy)ethyl)-3-(trifluoromethyl)-5-((2-(trimethylsilyl)ethoxy)methyl)-1,5-dihydro-4H-pyrrolo[2,3-d]pyridazin-4-one 1810 (100 mg, 0.15 mmol) in HCl-Dioxane (4 M, 40 mL) was stirred at rt for 16 h. The resulting reaction solution was basified (PH 8) by saturated aqueous NaHCO₃ at 0° C. and then extracted with EtOAc (50 mL×3). The combined organic layer was washed with brine, dried over Na₂SO₄ and concentrated under reduced pressure. The crude was purified by C18 column (Agela 40 g, mobile phase: ACN-H₂O (0.1% FA), gradient: 30-60) to give 1-(2-(3-oxo-3-(4-(5-(trifluoromethyl)pyrimidin-2-yl)piperazin-1-yl)propoxy)ethyl)-3-(trifluoromethyl)-1,5-dihydro-4H-pyrrolo[2,3-d]pyridazin-4-one 131 (21.5 mg, 98% purity, 23% yield) as white solid.

¹H NMR (400 MHz, DMSO-d₆, ppm) δ:12.56 (s, 1H), 8.73 (s, 2H), 8.43 (s, 1H), 8.01 (s, 1H), 4.44 (t, J=4.4 Hz, 2H), 3.82-3.71 (m, 6H), 3.66 (t, J=6.0 Hz, 2H), 3.55-3.45 (m, 4H), 2.54-2.52 (m, 2H).

LCMS (ESI) calcd for C₂₁H₂₁F₆N₇O₃ [M+H]⁺ m/z 534.16, found 534.

### 10. Synthesis of 1-(2-(3-oxo-3-(4-(5-(trifluoromethyl)pyrimidin-2 yl)piperazin-1-yl)propoxy)ethyl)-3-(trifluoromethyl)-1,5-dihydro-4H-pyrazolo[3,4-d]pyridazin-4-one (Compound 153)

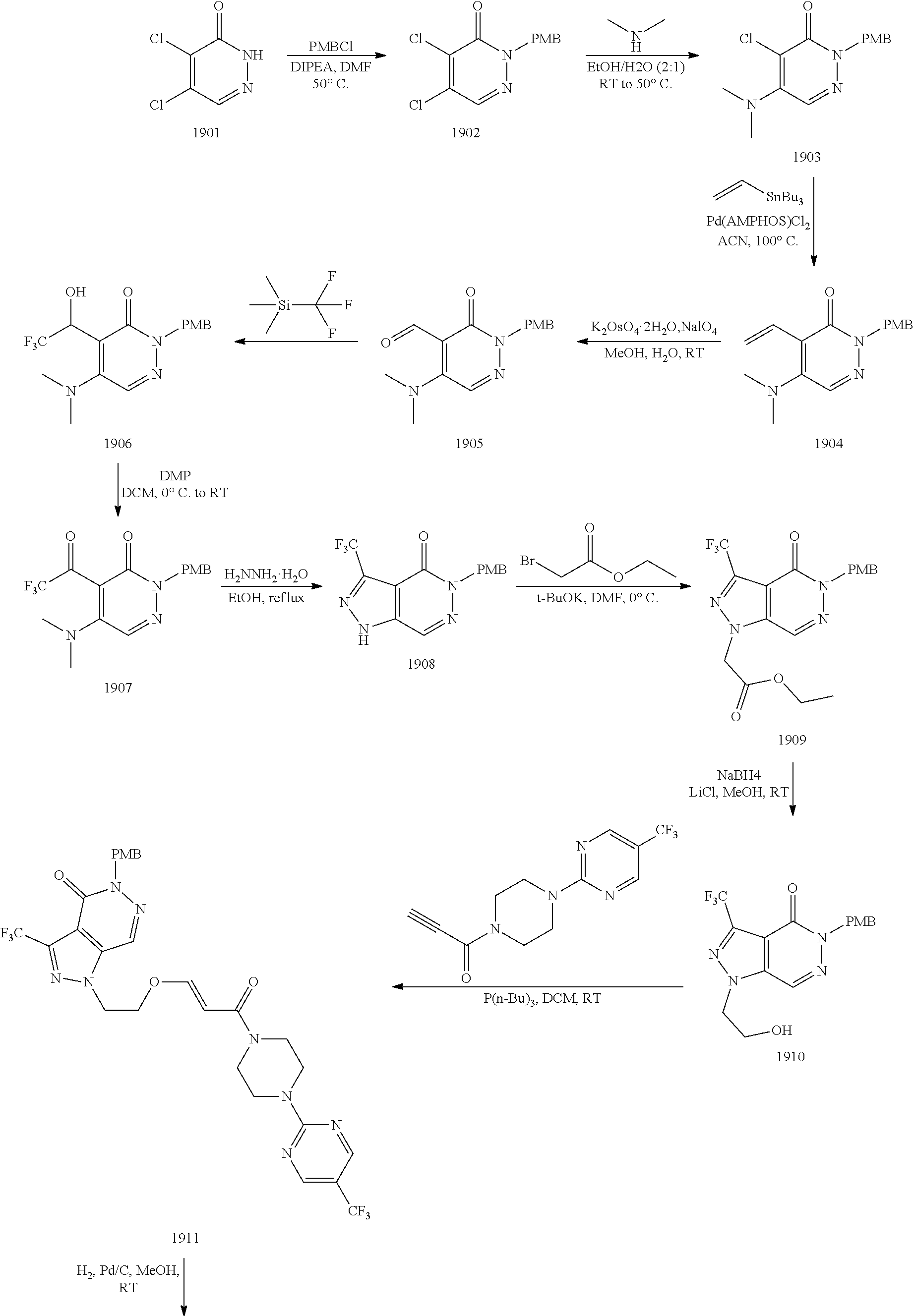

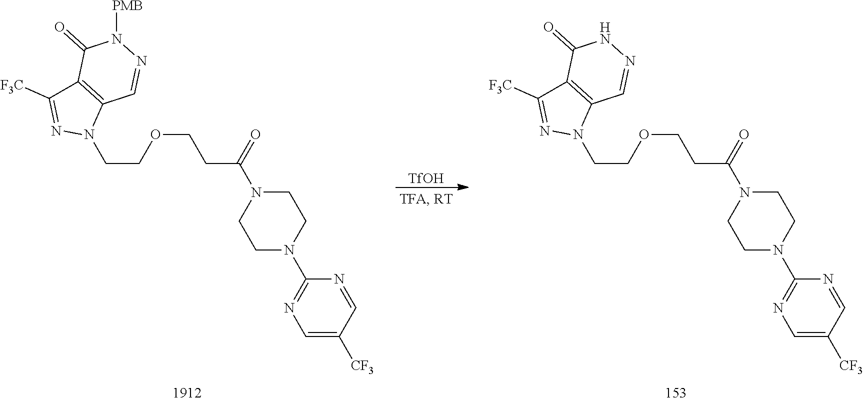

### Preparation of 4,5-dichloro-2-(4-methoxybenzyl) pyridazin-3(2H)-one (1902)

To a solution of 4,5-dichloropyridazin-3(2H)-one 1901 (8 g, 48.5 mmol) in DMF (100 mL) were added PMBCl (19 g, 121.3 mmol) and DIPEA (31.4 g, 242.5 mmol) at room temperature. The resulting mixture was stirred at 50° C. for 6 h. After cooling to room temperature, water (400 mL) was added, and the mixture was extracted EtOAc (3×150 mL). The combined organic layers were dried (Na₂SO₄), concentrated in vacuo. The residue was purified by flash chromatography (silica gel, eluting with PE/EtOAc=100:0 to 80:20) to obtain 4,5-dichloro-2-(4-methoxybenzyl) pyridazin-3(2H)-one 1902 (8 g, 90% purity, 52% yield) as white solid.

LCMS (ESI) calcd for C₁₂H₁₀Cl₂N₂O₂ [M+H]⁺ m/z 285.02, found 285.

### Preparation of 4-chloro-5-(dimethylamino)-2-(4-methoxybenzyl) pyridazin-3(2H)-one (1903)

To a solution of 4,5-dichloro-2-(4-methoxybenzyl) pyridazin-3(2H)-one 1902 (8 g, 28.1 mmol) in EtOH (100 mL) and H₂O (50 mL) was added dimethylamine in MeOH (2 M, 70.5 mL) at room temperature. The reaction mixture was stirred at 50° C. for 4 h. After cooling to room temperature, the mixture was concentrated in vacuo. Water (100 mL) was added, and the mixture was extracted EtOAc (3×150 mL). The combined organic layers were dried (Na₂SO₄), concentrated in vacuo, and purified by flash chromatography (silica gel, eluting with PE/EtOAc=100:0 to 20:80) to obtain 4-chloro-5-(dimethylamino)-2-(4-methoxybenzyl) pyridazin-3(2H)-one 1903 (8 g, 85% purity, 82% yield) as white solid.

LCMS (ESI) calcd for C₁₄H₁₆ClN₃O₂[M+H]⁺ m/z 294.1, found 294.

### Preparation of 5-(dimethylamino)-2-(4-methoxybenzyl)-4-vinvlpyridazin-3(2H)-one (1904)

To a solution of 4-chloro-5-(dimethylamino)-2-(4-methoxybenzyl) pyridazin-3(2H)-one 1903 (8 g, 27.2 mmol) in ACN (100 mL) were added tributyl(vinyl) stannane (17.3 g, 54.4 mmol) and Pd(AMPHOS)Cl₂ (1.93 g, 2.72 mmol) at room temperature. The resulting mixture was stirred at 100° C. for 10 h. After cooling to room temperature, the mixture was concentrated in vacuo. The residue was purified by flash chromatography (silica gel, eluting with PE/EtOAc=100:0 to 10:90) to obtain 5-(dimethylamino)-2-(4-methoxybenzyl)-4-vinylpyridazin-3(2H)-one 1904 (6 g, 90% purity, 69% yield) as white solid.

LCMS (ESI) calcd for C₁₆H₁₉N₃O₂ [M+H]⁺ m/z 286.16, found 286.

### Preparation of 5-(dimethylamino)-2-(4-methoxybenzyl)-3-oxo-2,3-dihydropyridazine-4-carbaldehyde (1905)

To a solution of 5-(dimethylamino)-2-(4-methoxybenzyl)-4-vinylpyridazin-3(2H)-one 1904 (3 g, 0.01 mol) in MeOH/H₂O (2:1, 30 mL) were added K₂OsO₄·2H₂O (0.39 g, 0.001 mol), NaIO₄ (8.9 g, 0.04 mol) at rt. The reaction mixture was stirred at rt for 1 h. The resulting mixture was quenched with water and extracted with DCM (50 mL×3). The combined organic layer was washed with brine and concentrated under reduced pressure. The residue was purified by silica gel column (eluting with PE/EtOAc=100:0 to 50:50) to give 5-(dimethylamino)-2-(4-methoxybenzyl)-3-oxo-2,3-dihydropyridazine-4-carbaldehyde 1905 (1.8 g, 90% purity, 53% yield) as yellow oil.

LCMS (ESI) calcd for C₁₅H₁₇N₃O₃[M+H]⁺ m/z 288.13, found 288.10.

### Preparation of 5-(dimethylamino)-2-(4-methoxybenzyl)-4-(2,2,2-trifluoro-1-hydroxyethyl)pyridazin-3(2H)-one (1906)

To a solution of 5-(dimethylamino)-2-(4-methoxybenzyl)-3-oxo-2,3-dihydropyridazine-4-carbaldehyde 1905 (1.8 g, 0.006 mol), trimethyl(trifluoromethyl)silane (0.9 g, 0.006 mol) in THE (20 mL) was added TBAF in THF (1 M, 0.6 mL) at rt. The reaction mixture was stirred at rt for 1 h. The resulting mixture was quenched with water and extracted with DCM (50 mL×3). The combined organic layer was washed with brine and concentrated under reduced pressure. The residue was purified by silica gel column (eluting with PE/EtOAc=100:0 to 30:70) to obtain 5-(dimethylamino)-2-(4-methoxybenzyl)-4-(2,2,2-trifluoro-1-hydroxyethyl)pyridazin-3(2H)-one 1906 (1.6 g, 90% purity, 63% yield) as yellow oil.

LCMS (ESI) calcd for C₁₆H₁₈F₃N₃O₃[M+H]⁺ m/z 358.13, found 358.10.

### Preparation of 5-(dimethylamino)-2-(4-methoxybenzyl)-4-(2,2,2-trifluoroacetyl)pyridazin-3(2H)-one (1907)

To a solution of 5-(dimethylamino)-2-(4-methoxybenzyl)-4-(2,2,2-trifluoro-1-hydroxyethyl)pyridazin-3(2H)-one 1906 (1.6 g, 0.004 mol) in DCM (30 mL) was added Dess-Martin periodinane (3.8 g, 0.009 mol) at rt. The reaction mixture was stirred at rt for 1 h. The resulting mixture was quenched with water and extracted with DCM (50 mL×3). The combined organic layer was washed with brine and concentrated under reduced pressure. The residue was purified by silica gel column (eluting with PE/EtOAc=100:0 to 50:50) to obtain 5-(dimethylamino)-2-(4-methoxybenzyl)-4-(2,2,2-trifluoroacetyl)pyridazin-3(2H)-one 1907 (1.5 g, 90% purity, 84% yield) as yellow oil.

LCMS (ESI) calcd for C₁₆H₁₆F₃N₃O₃[M+H]⁺ m/z 356.11, found 356.10.

### Preparation of 5-(4-methoxybenzyl)-3-(trifluoromethyl)-1,5-dihydro-4H-pyrazolo[3,4-d]pyridazin-4-one (1908)

To a solution of 5-(dimethylamino)-2-(4-methoxybenzyl)-4-(2,2,2-trifluoroacetyl)pyridazin-3(2H)-one 1907 (1.6 g, 0.004 mol) in EtOH (30 mL) was added H₂NNH₂·H₂O (80% wt, 1.0 g, 0.020 mol) at rt. The reaction mixture was stirred at 80° C. for 3 h. The resulting light brown solution was concentrated under reduced pressure to remove most EtOH to get crude. The crude was purified by C18 column (Agela 40 g, mobile phase: ACN-H₂O (0.1% FA), gradient: 30-60) to give 5-(4-methoxybenzyl)-3-(trifluoromethyl)-1,5-dihydro-4H-pyrazolo[3,4-d]pyridazin-4-one 1908 (0.9 g, 90% purity, 55% yield) as white solid.

LCMS (ESI) calcd for C₁₄H₁₁F₃N₄O₂ [M+H]⁺ m/z 325.08, found 325.05.

### Preparation of ethyl 2-(5-(4-methoxybenzyl)-4-oxo-3-(trifluoromethyl)-4,5-dihydro-1H-pyrazolo[3,4-d]pyridazin-1-yl)acetate (1909)

To a solution of 5-(4-methoxybenzyl)-3-(trifluoromethyl)-1,5-dihydro-4H-pyrazolo[3,4-d]pyridazin-4-one 1908 (200 mg, 0.61 mmol), ethyl 2-bromoacetate (308 mg, 1.84 mmol) was added t-BuOK (206 mg, 1.84 mmol) at 0° C. The reaction mixture was stirred at rt for 1 h. The reaction solution was quenched with water and extracted with EtOAc (20 mL×3). The organic phase was concentrated and purified by silica gel column (eluting with PE/EtOAc=100:0 to 50:50) to obtain ethyl 2-(5-(4-methoxybenzyl)-4-oxo-3-(trifluoromethyl)-4,5-dihydro-1H-pyrazolo[3,4-d]pyridazin-1-yl)acetate 1909 (200 mg, 90% purity, 71% yield) as yellow oil.

LCMS (ESI) calcd for C₁H₁₇F₃N₄O₄[M+H]⁺ m/z 411.12, found 411.10.

### Preparation of 1-(2-hydroxyethyl)-5-(4-methoxybenzyl)-3-(trifluoromethyl)-1,5-dihydro-4H-pyrazolo[3,4-d]pyridazin-4-one (1910)

To a solution of ethyl 2-(5-(4-methoxybenzyl)-4-oxo-3-(trifluoromethyl)-4,5-dihydro-1H-pyrazolo[3,4-d]pyridazin-1-yl)acetate 1909 (200 mg, 0.48 mmol) in MeOH (20 mL) were added LiCl (82 mg, 1.94 mmol), NaBH₄ (73 mg, 1.94 mmol) at rt. The reaction mixture was stirred at rt for 1 h. The resulting mixture was quenched with water and extracted with EtOAc (50 mL×3). The combined organic layer was washed with brine and concentrated under reduced pressure. The residue was purified by silica gel column (eluting with PE/EtOAc=100:0 to 50:50) to obtain 1-(2-hydroxyethyl)-5-(4-methoxybenzyl)-3-(trifluoromethyl)-1,5-dihydro-4H-pyrazolo[3,4-d]pyridazin-4-one 1910 (140 mg, 90% purity, 70% yield) as yellow oil.

LCMS (ESI) calcd for C₁₆H₁₅F₃N₄O₃[M+H]⁺ m/z 369.11, found 369.05.

### Preparation of (E)-5-(4-methoxybenzyl)-1-(2-((3-oxo-3-(4-(5-(trifluoromethyl)pyrimidin-2-yl)piperazin-1-yl)prop-1-en-1-yl)oxy)ethyl)-3-(trifluoromethyl)-1,5-dihydro-4H-pyrazolo[3,4-d]pyridazin-4-one (1911)

To a solution of 1-(2-hydroxyethyl)-5-(4-methoxybenzyl)-3-(trifluoromethyl)-1,5-dihydro-4H-pyrazolo[3,4-d]pyridazin-4-one 1910 (140 mg, 0.37 mmol), 1-(4-(5-(trifluoromethyl)pyrimidin-2-yl)piperazin-1-yl)prop-2-yn-1-one (108 mg, 0.37 mmol) in DCM (10 mL) was added P(n-Bu)₃ (15 mg, 0.075 mmol) at rt. The reaction mixture was stirred at rt for 1 h. The resulting mixture was quenched with water and extracted with DCM (50 mL×3). The combined organic layer was washed with brine and concentrated under reduced pressure. The residue was purified by silica gel column (eluting with PE/EtOAc=100:0 to 0:100) to obtain (E)-5-(4-methoxybenzyl)-1-(2-((3-oxo-3-(4-(5-(trifluoromethyl)pyrimidin-2-yl)piperazin-1-yl)prop-1-en-1-yl)oxy)ethyl)-3-(trifluoromethyl)-1,5-dihydro-4H-pyrazolo[3,4-d]pyridazin-4-one 1911 (200 mg, 90% purity, 72% yield) as yellow oil.

LCMS (ESI) calcd for C₂₈H₂₆F₆N₈O₄[M+H]⁺ m/z 653.20, found 653.

### Preparation of 5-(4-methoxybenzyl)-1-(2-(3-oxo-3-(4-(5-(trifluoromethyl)pyrimidin-2-yl)piperazin-1-yl)propoxy)ethyl)-3-(trifluoromethyl)-1,5-dihydro-4H-pyrazolo[3,4-d]pyridazin-4-one (1912)

A solution of (E)-5-(4-methoxybenzyl)-1-(2-((3-oxo-3-(4-(5-(trifluoromethyl)pyrimidin-2-yl)piperazin-1-yl)prop-1-en-1-yl)oxy)ethyl)-3-(trifluoromethyl)-1,5-dihydro-4H-pyrazolo[3,4-d]pyridazin-4-one 1911 (150 mg, 0.22 mmol) and Pd/C (30 mg) in MeOH/DCM (15 mL, 2:1) was stirred at rt for 1 h under H₂ atmosphere. The resulting solution was filtered through diatomaceous earth and the filter cake was washed with DCM (5 mL×4). The filtrate was concentrated under reduced pressure to give 5-(4-methoxybenzyl)-1-(2-(3-oxo-3-(4-(5-(trifluoromethyl)pyrimidin-2-yl)piperazin-1-yl)propoxy)ethyl)-3-(trifluoromethyl)-1,5-dihydro-4H-pyrazolo[3,4-d]pyridazin-4-one 1912 (140 mg, 90% purity, 83% yield) as yellow oil.

LCMS (ESI) calcd for C₂₈H₂₈F₆N₈O₄[M+H]⁺ m/z 655.21, found 655.

### Preparation of 1-(2-(3-oxo-3-(4-(5-(trifluoromethyl)pyrimidin-2-yl)piperazin-1-yl)propoxy)ethyl)-3-(trifluoromethyl)-1,5-dihydro-4H-pyrazolo[3,4-d]pyridazin-4-one (Compound 153)

To a solution of 5-(4-methoxybenzyl)-1-(2-(3-oxo-3-(4-(5-(trifluoromethyl)pyrimidin-2-yl)piperazin-1-yl)propoxy)ethyl)-3-(trifluoromethyl)-1,5-dihydro-4H-pyrazolo[3,4-d]pyridazin-4-one 1912 (140 mg, 0.21 mmol) in TFA (10 mL) was added TfOH (64 mg, 0.42 mmol) at rt. The reaction mixture was stirred at rt for 1 h. The resulting mixture was diluted with DCM (50 mL) and then adjusted PH to 8 with saturated aqueous NaHCO₃ at 0° C. The basified solution was extracted with DCM (10 mL×3). The combined organic layer was washed with brine, dried over Na₂SO₄ and concentrated under reduced pressure. The crude product was purified by C18 column (Agela 40 g, mobile phase: ACN-H₂O (0.1% FA), gradient: 30-60) to give 1-(2-(3-oxo-3-(4-(5-(trifluoromethyl)pyrimidin-2-yl)piperazin-1-yl)propoxy)ethyl)-3-(trifluoromethyl)-1,5-dihydro-4H-pyrazolo[3,4-d]pyridazin-4-one 153 (47 mg, 99% purity, 40% yield) as white solid.

¹H NMR (400 MHz, DMSO-d₆, ppm) δ:12.85 (s, 1H), 8.73 (s, 2H), 8.58 (s, 1H), 4.70 (t, J=4.8 Hz, 2H), 3.82 (t, J=5.0 Hz, 2H), 3.79-3.73 (m, 4H), 3.63 (t, J=6.2 Hz, 2H), 3.50-3.43 (m, 4H), 2.49-2.46 (m, 2H).

LCMS (ESI) calcd for C₂₀H₂₀F₆N₈O₃[M+H]⁺ m/z 535.16, found 535.20.

### 11. Synthesis of 7-chloro-5-(2-(3-oxo-3-(4-(5-(trifluoromethyl)pyrimidin-2-yl)piperazin-1-yl)propoxy)ethyl)phthalazin-1(2H)-one (Compound 80)

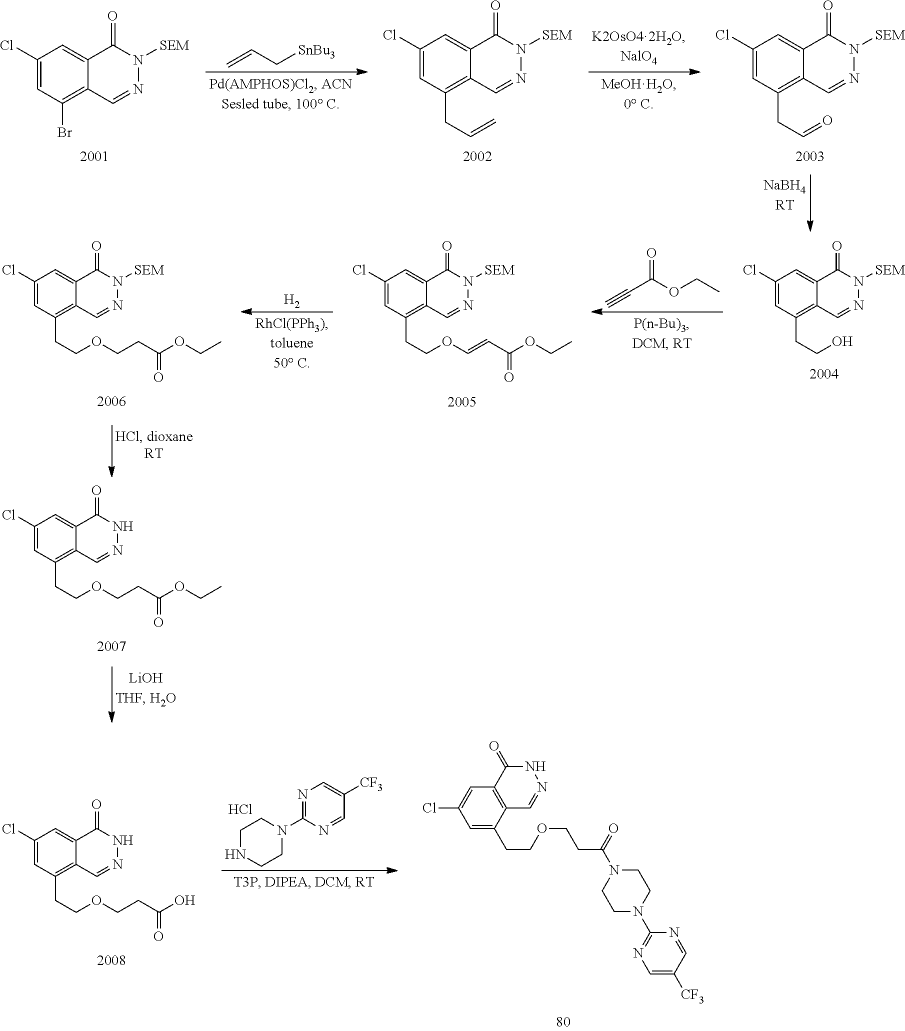

### Preparation of 5-allyl-7-chloro-2-((2-(trimethylsilyl)ethoxy)methyl)phthalazin-1(2H)-one (2002)

To a solution of 5-bromo-7-chloro-2-((2-(trimethylsilyl)ethoxy)methyl)phthalazin-1(2H)-one 2001 (400 mg, 1.02 mmol) in ACN (10 mL) were added allyltributylstannane (339 mg, 1.02 mmol) and Pd(AMPHOS)Cl₂ (73 mg, 0.10 mmol) at rt under N₂. The reaction mixture was stirred at 100° C. for 1 h in a sealed tube. The reaction mixture was concentrated under reduced pressure. The residue was purified by flash column chromatography (eluting with PE/EtOAc=80:20 to 70:30) to obtain 5-allyl-7-chloro-2-((2-(trimethylsilyl)ethoxy)methyl)phthalazin-1(2H)-one 2002 (300 mg, 90% purity, 75% yield) as colorless oil.

LCMS (ESI) calcd for C₁₇H₂₃ClN₂O₂Si [M+H]⁺ m/z 351.12, found 351.15

### Preparation of 2-(7-chloro-1-oxo-2-((2-(trimethylsilyl)ethoxy)methyl)-1,2-dihydrophthalazin-5-yl)acetaldehyde (2003)

To a solution of 5-allyl-7-chloro-2-((2-(trimethylsilyl)ethoxy)methyl)phthalazin-1(2H)-one 2002 (1.0 g, 2.8 mmol) in MeOH/H₂O (3:1, 40 mL) were added K₂OsO₄·2H₂O (100 mg, 0.28 mmol) and NaIO₄ (2.4 g, 11.2 mmol) at 0° C. successively. The reaction mixture was stirred at rt for 1 h. The resulting mixture was quenched with water and extracted with EtOAc (50 mL×3). The combined organic layer was washed with brine and concentrated under reduced pressure to give crude 2-(7-chloro-1-oxo-2-((2-(trimethylsilyl)ethoxy)methyl)-1,2-dihydrophthalazin-5-yl)acetaldehyde 2003 (500 mg, 80% purity, 39% yield) as yellow oil.

LCMS (ESI) calcd for C₁₆H₂₁ClN₂O₃Si [M−27]⁺ m/z 325.10, found 325.

### Preparation of 7-chloro-5-(2-hydroxyethyl)-2-((2-(trimethylsilyl)ethoxy)methyl)phthalazin-1(2H)-one (2004)

To a solution of 2-(7-chloro-1-oxo-2-((2-(trimethylsilyl)ethoxy)methyl)-1,2-dihydrophthalazin-5-yl)acetaldehyde 2003 (500 mg, 1.41 mmol) in methanol was added NaBH₄ (160 mg, 4.24 mmol) in portions at rt. The reaction mixture was stirred at rt for 1 h. The resulting mixture was quenched with water and extracted with EtOAc (50 mL×3). The combined organic layer was washed with brine and concentrated under reduced pressure. The residue was purified by silica gel column (eluting with PE/EtOAc=100:0 to 60:40) to obtain 7-chloro-5-(2-hydroxyethyl)-2-((2-(trimethylsilyl)ethoxy)methyl)phthalazin-1(2H)-one 2004 (413 mg, 90% purity, 74% yield) as a white solid.

LCMS (ESI) calcd for C₁₆H₂₃ClN₂O₃Si [M+H]⁺ m/z 355.12, found 355.

### Preparation of ethyl (E)-3-(2-(7-chloro-1-oxo-2-((2-(trimethylsilyl)ethoxy)methyl)-1,2-dihydrophthalazin-5-yl)ethoxy)acrylate (2005)

To a solution of 7-chloro-5-(2-hydroxyethyl)-2-((2-(trimethylsilyl)ethoxy)methyl)phthalazin-1(2H)-one 2004 (410 mg, 1.16 mmol) and ethyl propiolate (114 mg, 1.16 mmol) in DCM (20 mL) was added P(n-Bu)₃ (117 mg, 0.58 mmol) at rt. The reaction mixture was stirred at rt for 1 h. The resulting mixture was quenched with water and extracted with DCM (50 mL×3). The combined organic layer was washed with brine and concentrated under reduced pressure. The residue was purified by flash column chromatography (eluting with PE/EtOAc=100:0 to 60:40) to obtain ethyl (E)-3-(2-(7-chloro-1-oxo-2-((2-(trimethylsilyl)ethoxy)methyl)-1,2 dihydrophthalazin-5-yl)ethoxy)acrylate 2005 (262 mg, 90% purity, 45% yield) as white solid.

LCMS (ESI) calcd for C₂₁H₂₉ClN₂O₅Si [M+Na]⁺ m/z 475.15, found 475.

### Preparation of ethyl 3-(2-(7-chloro-1-oxo-2-((2-(trimethylsilyl)ethoxy)methyl)-1,2-dihydrophthalazin-5-yl)ethoxy)propanoate (2006)

To a solution of ethyl (E)-3-(2-(7-chloro-1-oxo-2-((2-(trimethylsilyl)ethoxy)methyl)-1,2 dihydrophthalazin-5-yl)ethoxy)acrylate 2005 (240 mg, 0.53 mmol) in toluene(10 mL) was added RhCl(PPh₃)₃(25 mg) at rt. The reaction mixture was stirred at 50° C. for 2 h under H₂ atmosphere. The reaction solution was concentrated under reduced pressure. The residue was purified by flash column chromatography (eluting with PE/EtOAc=100:0 to 50:50) to obtain ethyl 3-(2-(7-chloro-1-oxo-2-((2-(trimethylsilyl)ethoxy)methyl)-1,2-dihydrophthalazin-5-yl)ethoxy)propanoate 2006 (250 mg, 90% purity, 93% yield) as colorless oil.

LCMS (ESI) calcd for C₂₁H₃₁ClN₂O₅Si [M+Na]⁺ m/z 477.17, found 477.

### Preparation of ethyl 3-(2-(7-chloro-1-oxo-1,2-dihydrophthalazin-5-yl)ethoxy)propanoate (2007)

A solution of ethyl 3-(2-(7-chloro-1-oxo-2-((2-(trimethylsilyl)ethoxy)methyl)-1,2-dihydrophthalazin-5-yl)ethoxy)propanoate 2006 (350 mg, 0.77 mmol) in HCl-Dioxane (4 M, 20 mL) was stirred at rt for 16 h. The mixture was concentrated under reduced pressure to obtain crude ethyl 3-(2-(7-chloro-1-oxo-1,2-dihydrophthalazin-5-yl)ethoxy)propanoate 2007 (152 mg, 90% purity, 55% yield) as colorless oil.

LCMS (ESI) calcd for C₁₅H₁₇ClN₂O₄ [M+H]⁺ m/z 325.09, found 325.

### Preparation of 3-(2-(7-chloro-1-oxo-1,2-dihydrophthalazin-5-yl)ethoxy)propanoic acid (2008)

To a solution of ethyl 3-(2-(7-chloro-1-oxo-1,2-dihydrophthalazin-5-yl)ethoxy)propanoate 2007 (150 mg, 0.46 mmol) in THF/H₂O (15 mL, 1:1) was added LiOH (58 mg, 0.14 mmol). The mixture was stirred at room temperature for 2 h. THE was removed under reduced pressure and the aqueous phase was acidified with 1 M HCl aq. to pH=4˜5. The resulting solid was collected by filtration and dried in vacuo to obtain 3-(2-(7-chloro-1-oxo-1,2-dihydrophthalazin-5-yl)ethoxy)propanoic acid 2008 (58 mg, 95% purity, 40% yield) as a white solid.

LCMS (ESI) calcd for C₁₃H₁₃ClN₂O₄ [M+H]⁺ m/z 297.06, found 297.

### Preparation of 7-chloro-5-(2-(3-oxo-3-(4-(5-(trifluoromethyl)pyrimidin-2-yl)piperazin-1-yl)propoxy)ethyl)phthalazin-1(2H)-one (Compound 80)

To a solution 3-(2-(7-chloro-1-oxo-1,2-dihydrophthalazin-5-yl)ethoxy)propanoic acid 2008 (29 mg, 0.10 mmol), 2-(piperazin-1-yl)-5-(trifluoromethyl) pyrimidine hydrochloride (25 mg, 0.11 mmol) in DCM (5 mL) were added DIPEA (51 mg, 0.39 mmol), T3P (50% wt in EtOAc, 124 mg, 0.20 mmol) at rt. The reaction mixture was stirred at rt for 1 h. The reaction solution was quenched with water (20 mL) and extracted with DCM (20 mL×3). The organic phase was concentrated and purified by prep-HPLC (columns: Gemini 5 um C18 150×21.2 mm, mobile phase: ACN-H₂O (0.1% FA), gradient: 40-95) to obtain 7-chloro-5-(2-(3-oxo-3-(4-(5-(trifluoromethyl)pyrimidin-2-yl)piperazin-1-yl)propoxy)ethyl)phthalazin-1(2H)-one 80 (19.3 mg, 99% purity, 38% yield) as a white solid.

¹H NMR (400 MHz, DMSO-d₆, ppm) δ: 12.82 (s, 1H), 8.73 (d, J=0.8 Hz, 2H), 8.51 (s, 1H), 8.03 (d, J=2.0 Hz, 1H), 7.86 (d, J=2.0 Hz, 1H), 3.81-3.74 (m, 4H), 3.73-3.63 (m, 4H), 3.54-3.48 (m, 4H), 3.25 (t, J=6.2 Hz, 2H), 2.56 (t, J=6.4 Hz, 2H).

LCMS (ESI) calcd for C₂₂H₂₂ClF₃N₆O₃ [M+H]⁺ m/z 511.14, found 511.

### 12. Synthesis of 5-(2-(3-oxo-3-(4-(5-(trifluoromethyl)pyridin-2-yl)piperazin-1-yl)propoxy)ethyl)phthalazin-1(2H)-one (Compound 98)

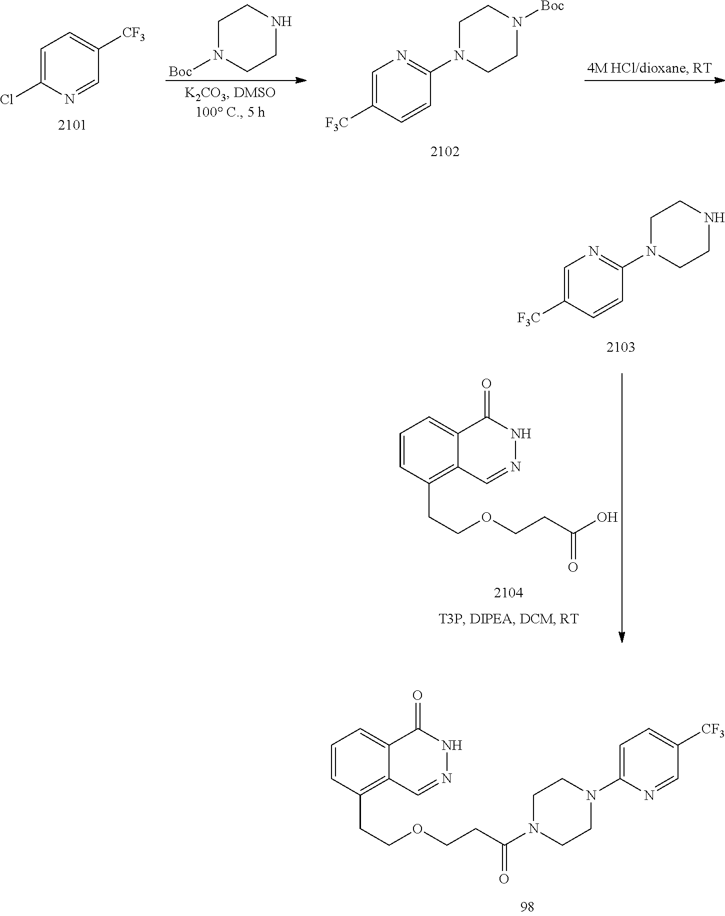 [SMILES: FC(F)(F)c1ccc(Cl)nc1 ; CC(C)(C)OC(=O)N1CCNCC1 ; CC(C)(C)OC(=O)N1CCN(c2ccc(C(F)(F)F)cn2)CC1 ; FC(F)(F)c1ccc(N2CCNCC2)nc1 ; O=C(O)CCOCCc1cccc2c(=O)[nH]ncc12 ; O=C(CCOCCc1cccc2c(=O)[nH]ncc12)N1CCN(c2ccc(C(F)(F)F)cn2)CC1]

### Preparation of tert-butyl 4-(5-(trifluoromethyl)pyridin-2-yl)piperazine-1-carboxylate (2102)

To a stirred mixture of 2-chloro-5-(trifluoromethyl)pyridine 2101 (500 mg, 2.739 mmol) in DMSO (10 mL) was added tert-butyl piperazine-1-carboxylate (513 mg, 2.739 mmol) and potassium carbonate (946 mg, 6.8475 mmol). The resulting mixture was stirred 5 hours at 100° C. The reaction was monitored by TLC. After the reaction was completed, the mixture was cooled to room temperature and diluted with ethyl acetate (30 mL). the reaction was filtered and the filtrate was washed with saturated NaHCO₃ solution (2×10 mL). The organic phase was dried over Na₂SO₄, filtered and concentrated under reduced pressure. The residue obtained was purified by silica gel column chromatography (eluting with PE/EtOAc=100:0 to 85:15). to afford tert-butyl 4-(5-(trifluoromethyl)pyridin-2-yl)piperazine-1-carboxylate 2102 (600 mg, 90% purity, 59% yield) as a white solid.

1H NMR (400 MHz, DMSO-d6, ppm) δ: 8.42 (s, 1H), 7.82 (dd, J=9.1, 2.5 Hz, 1H), 6.95 (d, J=9.1 Hz, 1H), 3.63 (dd, J=6.3, 4.3 Hz, 4H), 3.49-3.36 (m, 4H), 1.42 (s, 9H).

### Preparation of 1-(5-(trifluoromethyl)pyridin-2-yl)piperazine (2103)

A mixture of tert-butyl 4-(5-(trifluoromethyl)pyridin-2-yl)piperazine-1-carboxylate 2102 (300 mg, 0.9 mmol) was added to 4M HCl/dioxane (10 mL), the mixture was stirred for 5 hours at 20° C. The reaction was monitored by LCMS. After the reaction was completed, the reaction mixture was concentrated under reduced pressure to afford 1-(5-(trifluoromethyl)pyridin-2-yl)piperazine 2103 (210 mg, 90% purity, 78% yield) as white solid. The crude mixture was directly used for the next step without further purification.

LCMS: (ESI) calcd for C₁₀H₁₂F₃N₃ [M+H]⁺ m/z 232.10, found 232.

### Preparation of 5-(2-(3-oxo-3-(4-(5-(trifluoromethyl)pyridin-2-yl)piperazin-1-yl)propoxy)ethyl)phthalazin-1(2H)-one (Compound 98)

To a stirred solution of 1-(5-(trifluoromethyl)pyridin-2-yl)piperazine 2103 (60 mg, 0.2233 mmol) in DCM (5 mL) was added 3-[2-(1-oxo-2H-phthalazin-5-yl)ethoxy]propanoic acid 2104 (59 mg, 0.2233 mmol), T₃P (107 mg, 0.3349 mmol) and DIPEA (87 mg, 0.6699 mmol). The reaction mixture was stirred for 2 hours at 20° C. The reaction was monitored by LCMS. After the reaction was completed, the resulting mixture was diluted with water and extracted with DCM (30 mL×3). The combined organic layer was dried over Na₂SO₄ and concentrated under reduced pressure. The residue was purified by pre-HPLC (columns: Gemini 5 um C18 150*21.2 mm, mobile phase: ACN-H₂O (0.1% FA), gradient: 20-95) to obtain 5-(2-(3-oxo-3-(4-(5-(trifluoromethyl)pyridin-2-yl)piperazin-1-yl)propoxy)ethyl)phthalazin-1(2H)-one 98 (51.8 mg, 95% purity, 46% yield) as a white solid.

¹H NMR: (400 MHz, DMSO) δ 12.66 (s, 1H), 8.51 (s, 1H), 8.43 (s, 1H), 8.10 (d, J=7.7 Hz, 1H), 7.89-7.62 (m, 3H), 6.94 (d, J=9.1 Hz, 1H), 3.68 (m, 4H), 3.61 (m, 4H), 3.53 (s, 4H), 3.24 (t, J=5.9 Hz, 2H), 2.57 (t, J=6.3 Hz, 2H).

LCMS: (ESI) calcd for C₂₃H₂₄F₃N₅O₃[M+H]⁺ m/z 476.18, found 476.

### 13. Preparation of 7-chloro-5-(1-(4-oxo-4-(4-(5-(trifluoromethyl)pyrimidin-2-yl)piperazin-1-yl)butoxy)ethyl)phthalazin-1(2H)-one (compounds 174 and 175)

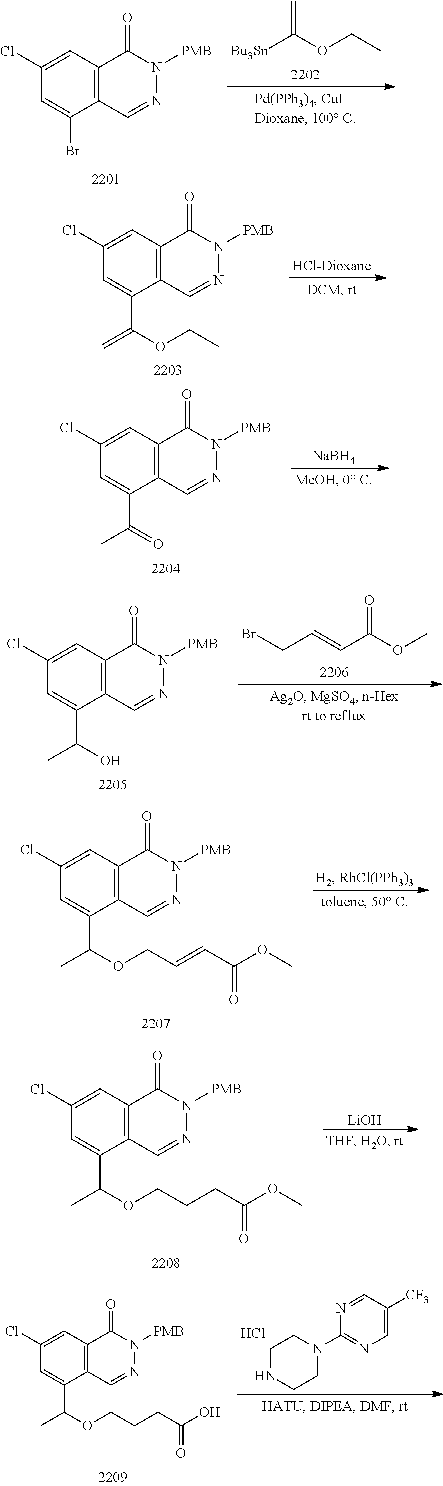

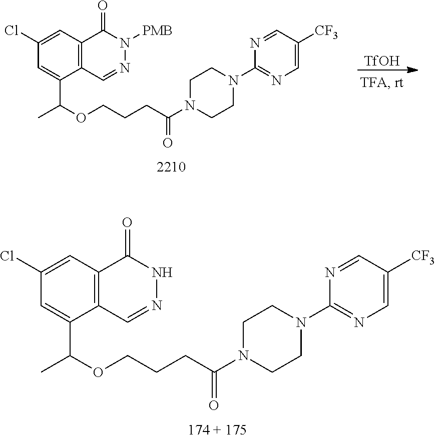

### Preparation of 7-chloro-5-(1-ethoxyvinyl)-2-(4-methoxybenzyl)phthalazin-1(2H)-one (2203)

To a solution of 5-bromo-7-chloro-2-(4-methoxybenzyl)phthalazin-1(2H)-one 2201 (620 mg, 1.63 mmol) in Dioxane (15 mL) at room temperature were added tributyl(1-ethoxyvinyl)stannane 2202 (141 mg, 1.63 mmol), CuI (31 mg, 0.16 mmol) and Pd(PPh₃)₄ (189 mg, 0.16 mmol) successively. The reaction mixture was stirred at 100° C. for 1 h under N₂ atmosphere. The reaction mixture was poured into cold water and then extracted with EtOAc (50 mL×3). The combined organic layer was dried over Na₂SO₄ and concentrated under reduced pressure. The residue was purified by flash column chromatography (eluting with PE/EtOAc=100:0 to 70:30) to obtained 7-chloro-5-(1-ethoxyvinyl)-2-(4-methoxybenzyl)phthalazin-1(2H)-one 2203 (410 mg, 90% purity, 61% yield) as a yellow solid.

LCMS (ESI) calcd for C₂₀H₁₉ClN₂O₃[M+H]⁺ m/z 371.11, found 371.15

### Preparation of 5-acetyl-7-chloro-2-(4-methoxybenzyl)phthalazin-1(2H)-one (2204)

A solution of 7-chloro-5-(1-ethoxyvinyl)-2-(4-methoxybenzyl)phthalazin-1(2H)-one 2203 (400 mg, 1.08 mmol) in DCM (5 mL) was added HCl-Dioxane (4 M, 5 mL) at rt. The reaction solution was stirred at rt for 4 h. The resulting reaction mixture was evaporated under reduced pressure and the residue was purified by flash column chromatography (eluting with PE/EtOAc=85:15 to 70:30) to afford 5-acetyl-7-chloro-2-(4-methoxybenzyl)phthalazin-1(2H)-one 2204 (347 mg, 90% purity, 84% yield) as a colorless oil.

LCMS (ESI) calcd for C₁₈H₁₅ClN₂O₃[M+H]⁺ m/z 343.08, found 343.10

### Preparation of 7-chloro-5-(1-hydroxyethyl)-2-(4-methoxybenzyl)phthalazin-1(2H)-one (2205)

To a solution of 5-acetyl-7-chloro-2-(4-methoxybenzyl)phthalazin-1(2H)-one 2204 (340 mg, 0.99 mmol) in MeOH (5 mL), NaBH₄ (75 mg, 1.98 mmol) was added in portions at 0° C. The reaction was then stirred at 0° C. for 10 min. The reaction solution was poured into water (30 mL) and extracted with EtOAc (30 mL×3). The combined organic layer was washed with saturated NH₄Cl solution, dried over Na₂SO₄ and concentrated under reduced pressure. The residue was purified by flash chromatography (eluting with PE/EtOAc=85:15 to 30:70) to give 7-chloro-5-(1-hydroxyethyl)-2-(4-methoxybenzyl)phthalazin-1(2H)-one 2205 (335 mg, 90% purity, 88% yield) as a white solid.

LCMS (ESI) calcd for C₁₈H₁₇ClN₂O₃[M+H]+m/z 345.09, found 345.10

### Preparation of methyl (E)-4-(1-(7-chloro-2-(4-methoxybenzyl)-1-oxo-1,2-dihydrophthalazin-5-yl)ethoxy)but-2-enoate (2207)

To a solution of 7-chloro-5-(1-hydroxyethyl)-2-(4-methoxybenzyl)phthalazin-1(2H)-one 2205 (330 mg, 0.96 mmol) in n-Hexane (15 mL) was added Ag₂O (887 mg, 3.83 mmol) followed by MgSO₄ (459 mg, 3.83 mmol) at room temperature. The reaction mixture was heated at 80° C. for 1 h then the solution was added Ag₂O (887 mg, 3.83 mmol) followed by methyl (E)-4-bromobut-2-enoate 2206 (857 mg, 4.79 mmol). The resulting solution was heated at reflux for 18 h. The reaction mixture was poured into cold water and then extracted with EtOAc (50 mL×4). The combined organic layer was dried over Na₂SO₄ and concentrated under reduced pressure. The residue was purified by flash chromatography (eluting with EtOAc/PE, 30% to 50%) to afford methyl (E)-4-(1-(7-chloro-2-(4-methoxybenzyl)-1-oxo-1,2-dihydrophthalazin-5-yl)ethoxy)but-2-enoate 2207 (85 mg, 90% purity, 18% yield) as a white solid.

LCMS (ESI) calcd for C₂₃H₂₃ClN₂O₅ [M+H]⁺ m/z 443.13, found 443.10

### Preparation of methyl 4-(1-(7-chloro-2-(4-methoxybenzyl)-1-oxo-1,2-dihydrophthalazin-5-yl)ethoxy)butanoate (2208)

To a solution of methyl (E)-4-(1-(7-chloro-2-(4-methoxybenzyl)-1-oxo-1,2-dihydrophthalazin-5-yl)ethoxy)but-2-enoate 2207 (95 mg, 0.21 mmol) in toluene (5 mL) was added RhCl(PPh₃)₃(10 mg) at rt. The reaction mixture was stirred at 50° C. for 12 h under H₂ atmosphere. After completion of reaction, the reaction mixture was concentrated under reduced pressure. The residue was purified by flash column chromatography on silica gel (eluting with PE/EtOAc=70/30 to 40/60) to obtain methyl 4-(1-(7-chloro-2-(4-methoxybenzyl)-1-oxo-1,2-dihydrophthalazin-5-yl)ethoxy)butanoate 2208 (70 mg, 90% purity, 66% yield) as a white solid.

LCMS (ESI) calcd for C₂₃H₂₅ClN₂O₅[M+H]⁺ m/z 445.15, found 445.20

### Preparation of 4-(1-(7-chloro-2-(4-methoxybenzyl)-1-oxo-1,2-dihydrophthalazin-5-yl)ethoxy) butanoic acid (2209)

To a solution of methyl 4-(1-(7-chloro-2-(4-methoxybenzyl)-1-oxo-1,2-dihydrophthalazin-5-yl)ethoxy)butanoate 2208 (70 mg, 0.16 mmol) in THF/H₂O (3/1, 5 mL) was added LiOH (20 mg, 0.47 mmol) at rt. The mixture was stirred at rt for 4 h. THE was removed under reduced pressure and the aqueous phase was acidified with 1 M aqueous HCl to pH=4˜5. The precipitate was collected by filtration and dried in vacuo to obtain 4-(1-(7-chloro-2-(4-methoxybenzyl)-1-oxo-1,2-dihydrophthalazin-5-yl)ethoxy)butanoic acid 2209 (55 mg, 90% purity, 73% yield) as a white solid.

LCMS (ESI) calcd for C₂₂H₂₃ClN₂O₅ [M+H]⁺ m/z 431.13, found 431.20

### Preparation of 7-chloro-2-(4-methoxybenzyl)-5-(1-(4-oxo-4-(4-(5-(trifluoromethyl)pyrimidin-2-yl)piperazin-1-yl)butoxy)ethyl)phthalazin-1(2H)-one (2210)

To a solution of 4-(1-(7-chloro-2-(4-methoxybenzyl)-1-oxo-1,2-dihydrophthalazin-5-yl)ethoxy)butanoic acid 2209 (50 mg, 0.12 mmol), 2-(piperazin-1-yl)-5-(trifluoromethyl)pyrimidine hydrochloride (47 mg, 0.17 mmol) in DMF (10 mL) were added DIPEA (60 mg, 0.46 mmol), HATU (87 mg, 0.23 mmol) successively at rt. The reaction mixture was stirred at rt for 1 h. The reaction solution was quenched with water (20 mL) and extracted with EtOAc (20 mL×3). The organic phase was washed with brine (20 mL×3). The combined organic layers were concentrated under reduced pressure. The residue was purified by flash column chromatography (eluting with PE/EtOAc=90:10 to 40:60) to obtain 7-chloro-2-(4-methoxybenzyl)-5-(1-(4-oxo-4-(4-(5-(trifluoromethyl)pyrimidin-2-yl)piperazin-1-yl)butoxy)ethyl)phthalazin-1(2H)-one 2210 (70 mg, 90% purity, 84% yield) as a white solid.

LCMS (ESI) calcd for C₃₁H₃₂ClF₃N₆O₄ [M+H]⁺ m/z 645.21, found 645.35

### Preparation of 7-chloro-5-(1-(4-oxo-4-(4-(5-(trifluoromethyl)pyrimidin-2-yl)piperazin-1-yl)butoxy)ethyl)phthalazin-1(2H)-one (mixture of 174 and 175)

To the solution of 7-chloro-2-(4-methoxybenzyl)-5-(1-(4-oxo-4-(4-(5-(trifluoromethyl)pyrimidin-2-yl)piperazin-1-yl)butoxy)ethyl)phthalazin-1(2H)-one 2210 (70 mg, 0.09 mmol) in TFA (2 mL) was added TfOH (28 mg, 0.19 mmol) at 0° C. The reaction solution was stirred at rt for 1 h. The mixture was adjusted to pH=8-9 with aqueous NaHCO₃ at 0° C., then the aqueous layer was extracted with EtOAc (50 mL×3). The combined organic layers were concentrated under reduced pressure. The residue was purified by C₁₈ column (mobile phase: ACN-H₂O (0.1% FA), gradient: 10%-95%) to give 7-chloro-5-(1-(4-oxo-4-(4-(5-(trifluoromethyl)pyrimidin-2-yl)piperazin-1-yl)butoxy)ethyl)phthalazin-1(2H)-one (mixture of 174 and 175) as a white solid.

### Chiral resolution of 7-chloro-5-(1-(4-oxo-4-(4-(5-(trifluoromethyl)pyrimidin-2-yl)piperazin-1-yl)butoxy)ethyl)phthalazin-1(2H)-one (174 and 175)

Compounds 174 and 175 were separated by SFC (Column: Daicel CHIRALPAK AS-H 250 mm×20 mm I.D., 5 um; Mobile phase: CO₂/MeOH (0.1% NH₃)=60/40) and concentrated under reduced pressure to afford the first fraction as 174 (5.9 mg, 99% purity, 100% ee, white solid) and the second fraction as 175 (8.2 mg, 99% purity, 98% ee, white solid).

### Compound 174

¹H NMR (400 MHz, DMSO-d₆, ppm) δ: 12.90 (s, 1H), 8.73 (s, 2H), 8.67 (s, 1H), 8.13 (d, J=2.0 Hz, 1H), 7.91 (d, J=2.0 Hz, 1H), 5.13 (q, J=6.4 Hz, 1H), 3.89-3.82 (m, 2H), 3.79 (t, J=5.2 Hz, 2H), 3.58-3.50 (m, 4H), 3.48-3.41 (m, 1H), 3.30-3.26 (m, 1H), 2.46-2.35 (m, 2H), 1.83-1.73 (m, 2H), 1.47 (d, J=6.4 Hz, 3H).

LCMS (ESI) calcd for C₂₃H₂₄ClF₃N₆O₃ [M+H]⁺ m/z 525.16, found 525.30

### Compound 175

¹H NMR (400 MHz, DMSO-d₆, ppm) δ: 12.90 (s, 1H), 8.73 (s, 2H), 8.67 (s, 1H), 8.13 (d, J=2.0 Hz, 1H), 7.91 (d, J=2.0 Hz, 1H), 5.13 (q, J=6.4 Hz, 1H), 3.89-3.82 (m, 2H), 3.79 (t, J=5.2 Hz, 2H), 3.58-3.50 (m, 4H), 3.48-3.41 (m, 1H), 3.30-3.26 (m, 1H), 2.46-2.35 (m, 2H), 1.83-1.73 (m, 2H), 1.47 (d, J=6.4 Hz, 3H).

LCMS (ESI) calcd for C₂₃H₂₄ClF₃N₆O₃ [M+H]⁺ m/z 525.16, found 525.30

### 14. Synthesis of 1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrazolo[3,4-d]pyridazin-4-one (compounds 176 and 177)

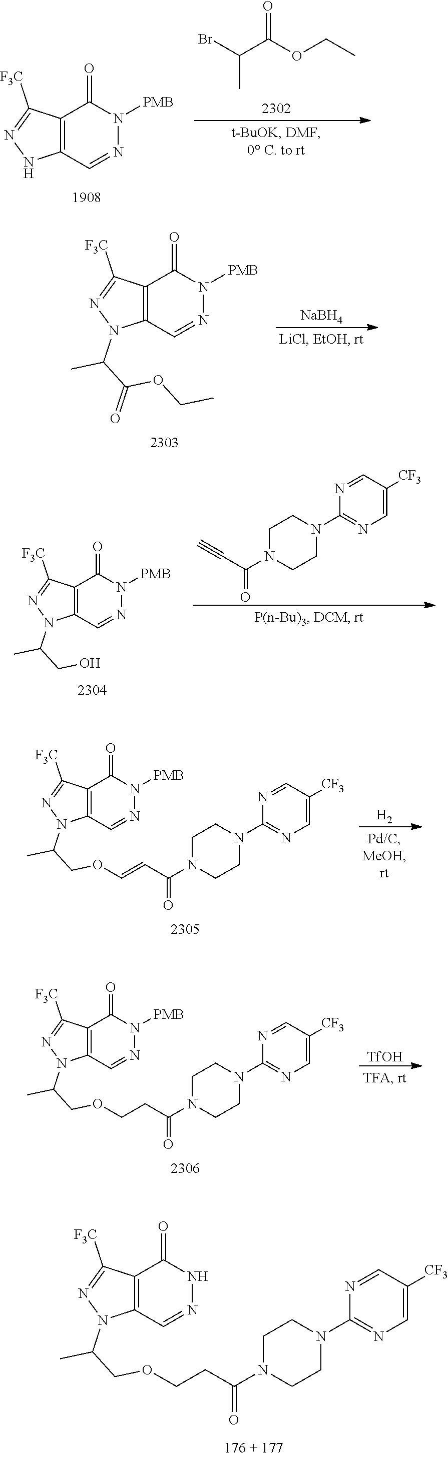

### Preparation of ethyl 2-(5-(4-methoxybenzyl)-4-oxo-3-(trifluoromethyl)-4,5-dihydro-1H-pyrazolo[3,4-d]pyridazin-1-yl)propanoate (2303)

To a solution of 5-(4-methoxybenzyl)-3-(trifluoromethyl)-1,5-dihydro-4H-pyrazolo[3,4-d]pyridazin-4-one 1908 (1 g, 0.0031 mol) was added t-BuOK (1.04 g, 0.0093 mmol) and ethyl 2-bromopropanoate 2302 (2.24 g, 0.0124 mol) at 0° C. The reaction mixture was stirred at rt for 6 h. The reaction solution was quenched with cold water and extracted with EtOAc (20 mL×3). The organic phase was concentrated under reduced pressure and purified by silica gel column (eluting with PE/EtOAc=100:0 to 50:50) to obtain ethyl 2-(5-(4-methoxybenzyl)-4-oxo-3-(trifluoromethyl)-4,5-dihydro-1H-pyrazolo[3,4-d]pyridazin-1-yl)propanoate 2303 (1 g, 90% purity, 68% yield) as a white solid.

LCMS (ESI) calcd for C₁₉H₁₉F₃N₄O₄[M+H]⁺ m/z 425.14, found 425.15.

### Preparation of 1-(1-hydroxypropan-2-yl)-5-(4-methoxybenzyl)-3-(trifluoromethyl)-1,5-dihydro-4H-pyrazolo[3,4-d]pyridazin-4-one (2304)

To a solution of ethyl 2-(5-(4-methoxybenzyl)-4-oxo-3-(trifluoromethyl)-4,5-dihydro-1H-pyrazolo[3,4-d]pyridazin-1-yl)propanoate 2303 (1 g, 0.00235 mol) in EtOH (30 mL) were added LiCl (0.394 g, 0.0094 mol) and NaBH₄ (0.355 g, 0.0094 mmol) at rt. The reaction mixture was stirred at rt for 1 h. The resulting mixture was quenched with water and extracted with EtOAc (50 mL×3). The combined organic layer was washed with brine and concentrated under reduced pressure. The residue was purified by silica gel column (eluting with PE/EtOAc=100:0 to 50:50) to obtain 1-(1-hydroxypropan-2-yl)-5-(4-methoxybenzyl)-3-(trifluoromethyl)-1,5-dihydro-4H-pyrazolo[3,4-d]pyridazin-4-one 2304 (0.8 g, 90% purity, 56% yield) as a white solid.

LCMS (ESI) calcd for C₁₇H₁₇F₃N₄O₃[M+H]⁺ m/z 383.13, found 383.15.

### Preparation of (E)-5-(4-methoxybenzyl)-1-(1-((3-oxo-3-(4-(5-(trifluoromethyl)pyrimidin-2-yl)piperazin-1-yl)prop-1-en-1-yl)oxy)propan-2-yl)-3-(trifluoromethyl)-1,5-dihydro-4H-pyrazolo[3,4-d]pyridazin-4-one (2305)

To a solution of 1-(1-hydroxypropan-2-yl)-5-(4-methoxybenzyl)-3-(trifluoromethyl)-1,5-dihydro-4H-pyrazolo[3,4-d]pyridazin-4-one 2304 (139 mg, 0.3626 mmol), 1-(4-(5-(trifluoromethyl)pyrimidin-2-yl)piperazin-1-yl)prop-2-yn-1-one (101 mg, 0.356 mmol) in DCM (15 mL) was added P(n-Bu)₃ (37 mg, 0.183 mmol) at rt. The reaction mixture was stirred at rt for 1 h. The resulting mixture was quenched with water and extracted with DCM (50 mL×3). The combined organic layer was washed with brine and concentrated under reduced pressure. The residue was purified by silica gel column (eluting with PE/EtOAc=100:0 to 0:100) to obtain (E)-5-(4-methoxybenzyl)-1-(1-((3-oxo-3-(4-(5-(trifluoromethyl)pyrimidin-2-yl)piperazin-1-yl)prop-1-en-1-yl)oxy)propan-2-yl)-3-(trifluoromethyl)-1,5-dihydro-4H-pyrazolo[3,4-d]pyridazin-4-one 2305 (200 mg, 90% purity, 74% yield) as a white solid.

LCMS (ESI) calcd for C₂₉H₂₈F₆N₈O₄[M+H]⁺ m/z 667.21, found 667.25.

### Preparation of 5-(4-methoxybenzyl)-1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrazolo[3,4-d]pyridazin-4-one (2306)

To a solution of (E)-5-(4-methoxybenzyl)-1-(1-((3-oxo-3-(4-(5-(trifluoromethyl)pyrimidin-2-yl)piperazin-1-yl)prop-1-en-1-yl)oxy)propan-2-yl)-3-(trifluoromethyl)-1,5-dihydro-4H-pyrazolo[3,4-d]pyridazin-4-one 2305 (200 mg, 0.300 mmol) in MeOH (10 mL) was added 10% Pd/C (20 mg). The mixture was evacuated and backfilled with hydrogen three times and then charged with hydrogen. The resulting mixture was stirred at room temperature for 2 hours. Then the mixture was filtered through celite and concentrated under vacuum to give crude 5-(4-methoxybenzyl)-1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrazolo[3,4-d]pyridazin-4-one 2306 (190 mg, 90% purity, 85% yield) which was used directly in next step without further purification.

LCMS (ESI) calcd for C₂₉H₃₀F₆N₈O₄[M+H]⁺ m/z 669.23, found 669.25.

### Preparation of 1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrazolo[3,4-d]pyridazin-4-one (mixture of 176 and 177)

To a solution of 5-(4-methoxybenzyl)-1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrazolo[3,4-d]pyridazin-4-one 2306 (190 mg, 0.284 mmol) in TFA (8 mL) was added TfOH (0.3 mL) at rt. The reaction solution was stirred at rt for 0.5 h. The mixture was adjusted to pH=8-9 with saturated aqueous NaHCO₃ at 0° C. The aqueous layer was extracted with EtOAc (50 mL×3). The combined organic layers were concentrated under reduced pressure. The residue was purified by C₁₈ column, (mobile phase: ACN-H₂O (0.1% FA), gradient: 10%-95%) to give 1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrazolo[3,4-d]pyridazin-4-one (mixture of 176 and 177) (90 mg) as a white solid.

Chiral resolution of 1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrazolo[3,4-d]pyridazin-4-one (176 and 177)

Compounds 176 and 177 were separated by SFC (Column: DAICEL AD-H 4.6 mmI.D.*250 mmL 5 um; Mobile phase CO₂/MEOH (0.1% FA)=65/35) and concentrated under reduced pressure to afford the first fraction as 176 (31.6 mg, 95% purity, 100% ee, white solid) and the second fraction as 177 (27.5 mg, 100% purity, 100% ee, white solid).

### Compound 176

¹H NMR (400 MHz, DMSO-d₆, ppm) δ:12.84 (s, 1H), 8.73 (s, 2H), 8.65 (s, 1H), 5.23-5.10 (m, 1H), 3.78-3.72 (m, 5H), 3.71-3.63 (m, 2H), 3.52 (m, 1H), 3.49-3.40 (m, 4H), 2.47-2.40 (m, 2H), 1.50 (d, J=6.8 Hz, 3H).

LCMS (ESI) calcd for C₂₁H₂₂F₆N₈O₃[M+H]⁺ m/z 549.17, found 549.25.

### Compound 177

¹H NMR (400 MHz, DMSO-d₆, ppm) δ: 12.84 (s, 1H), 8.73 (s, 2H), 8.65 (s, 1H), 8.49 (s, 0.2H), 5.23-5.10 (m, 1H), 3.78-3.72 (m, 5H), 3.71-3.63 (m, 2H), 3.52 (m, 1H), 3.49-3.40 (m, 4H), 2.47-2.40 (m, 2H), 1.50 (d, J=6.8 Hz, 3H).

LCMS (ESI) calcd for C₂₁H₂₂F₆N₈O₃[M+H]⁺ m/z 549.17, found 549.30.

### 15. Synthesis of 1-ethyl-3-(2-(3-oxo-3-(4-(5-(trifluoromethyl)pyrimidin-2-yl)piperazin-1-yl)propoxy)ethyl)-1,6-dihydro-7H-pyrrolo[2,3-d]pyridazin-7-one (compound 203)

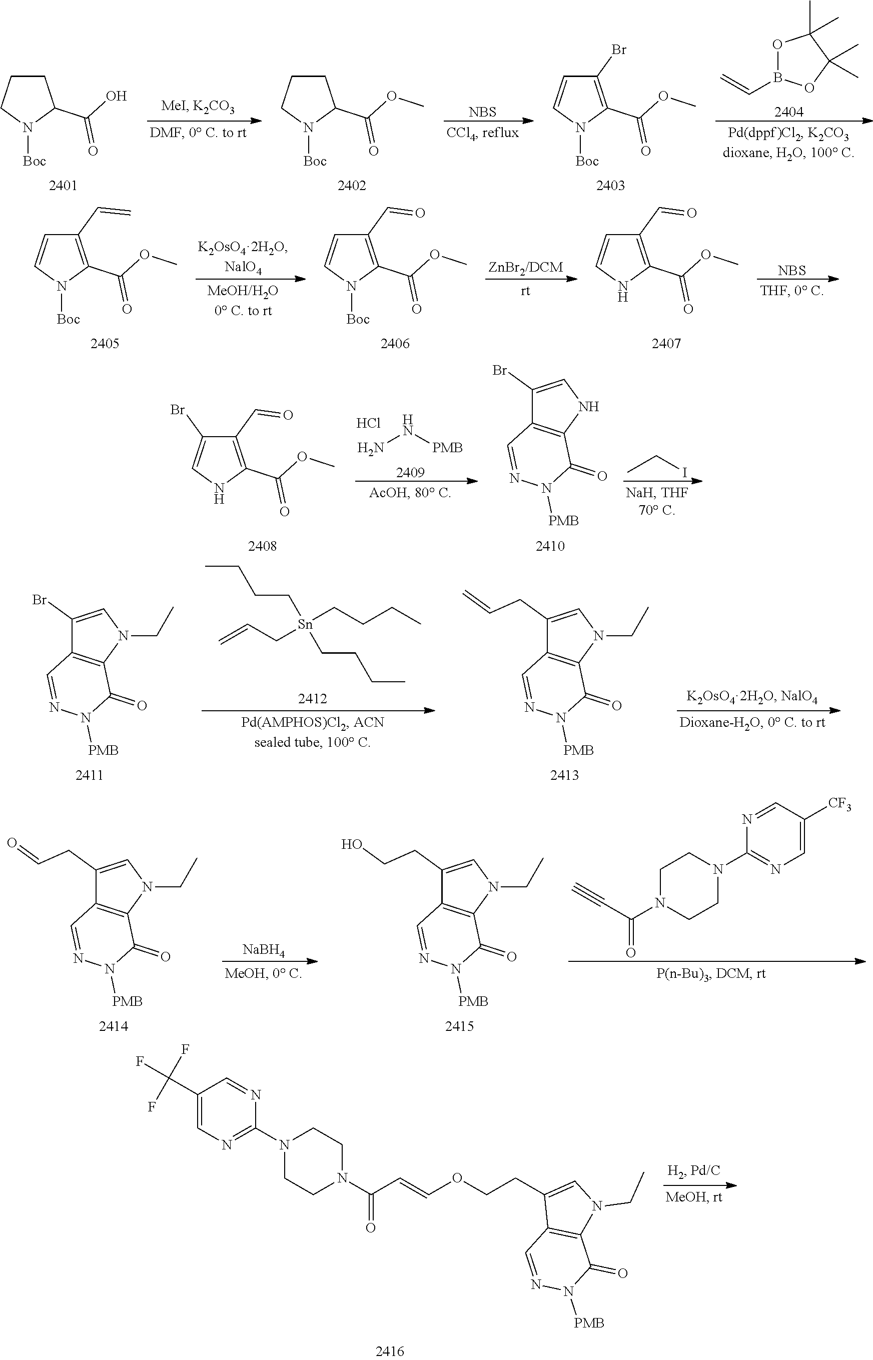

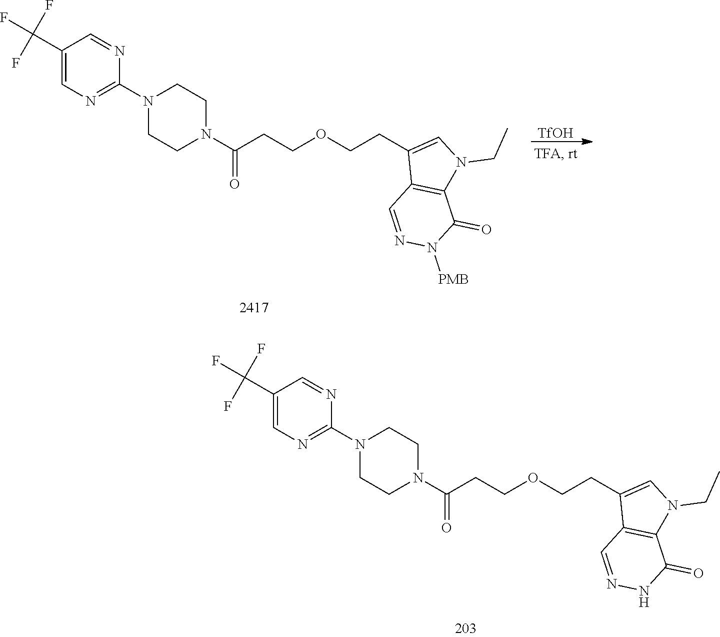

### Preparation of 1-(tert-butyl) 2-methyl pyrrolidine-1,2-dicarboxylate (2402)

To a solution of (tert-butoxycarbonyl)proline 2401 (74.0 g, 0.34 mol) in DMF (444 mL) at 0° C. were added K₂CO₃ (165 g, 1.20 mol) and MeI (97 g, 0.68 mol). The reaction mixture was stirred at rt for 18 h. The reaction solution was quenched with water and extracted with EtOAc (300 mL×3). The organic phase was concentrated and purified by silica gel column to obtain 1-(tert-butyl) 2-methyl pyrrolidine-1,2-dicarboxylate 2402 (65 g, 90% purity, 74% yield) as yellow oil.

¹H NMR (400 MHz, DMSO-d₆, ppm) δ: 4.23-4.09 (m, 1H), 3.68-3.60 (m, 3H), 3.38-3.31 (m, 2H), 2.27-2.11 (m, 1H), 1.88-1.74 (m, 3H), 1.42-1.28 (m, 9H).

### Preparation of 1-(tert-butyl) 2-methyl 3-bromo-1H-pyrrole-1,2-dicarboxylate (2403)

To a solution of 1-(tert-butyl) 2-methyl pyrrolidine-1,2-dicarboxylate 2402 (32.0 g, 0.139 mol) in CCl₄ (8 L) was added NBS (86.6 g, 0.486 mol). The reaction mixture was stirred at 85° C. for 1 h. The reaction solution was filtered and concentrated under reduced pressure. The residue was purified by silica gel column (eluting with PE/EtOAc=90:10) to obtain 1-(tert-butyl) 2-methyl 3-bromo-1H-pyrrole-1,2-dicarboxylate 2403 (22.8 g, 95% purity, 51% yield) as yellow oil.

LCMS (ESI) calcd for C₁₁H₁₄BrNO₄ [M−55]⁺ m/z 248.01, found 248.0

### Preparation of 1-(tert-butyl) 2-methyl 3-vinyl-1H-pyrrole-1,2-dicarboxylate (2405)

To a solution of 1-(tert-butyl) 2-methyl 3-bromo-1H-pyrrole-1,2-dicarboxylate 2403 (4.2 g, 0.014 mol) in 1,4-Dioxane/H₂O=5:1 (182 mL) at room temperature were added 4,4,5,5-tetramethyl-2-vinyl-1,3,2-dioxaborolane 2404 (5.31 g, 0.035 mol), K₂CO₃ (3.81 g, 0.028 mol) and Pd(dppf)Cl₂ (1.01 g, 0.0014 mol). The reaction mixture was stirred at 100° C. for 3 h under N₂. After cooling to rt, the reaction solution was quenched with water and extracted with EtOAc (100 mL×3). The organic phase was concentrated under reduced pressure. The residue was purified by silica gel column (eluting with PE/EtOAc=90:10 to 60:40) to obtain 1-(tert-butyl) 2-methyl 3-vinyl-1H-pyrrole-1,2-dicarboxylate 2405 (1.96 g, 80% purity, 45% yield) as yellow solid.

LCMS (ESI) calcd for C₁₃H₁₇NO₄ [M−55]⁺ m/z 196.12, found 195.95.

### Preparation of 1-(tert-butyl) 2-methyl 3-formyl-1H-pyrrole-1,2-dicarboxylate (2406)

To a solution of 1-(tert-butyl) 2-methyl 3-vinyl-1H-pyrrole-1,2-dicarboxylate 2405 (7.4 g, 0.03 mol) in MeOH/H₂O=3:1 (400 mL) at 0° C. was added K₂OsO₄·2H₂O (1.08 g, 0.003 mol) and NaIO₄ (25.08 g, 0.12 mol). The reaction mixture was stirred at rt for 1 h. The reaction solution was quenched with water and extracted with EtOAc (50 mL×3). The organic phase was concentrated under reduced pressure. The residue was purified by silica gel column (eluting with PE/EtOAc=90:10 to 0:100) to obtain 1-(tert-butyl) 2-methyl 3-formyl-1H-pyrrole-1,2-dicarboxylate 2406 (3.5 g, 95% purity, 44% yield) as yellow oil.

LCMS (ESI) calcd for C₁₂H₁₅NO₅ [M−99]⁺ m/z 154.10, found 154.15.

### Preparation of methyl 3-formyl-1H-pyrrole-2-carboxylate (2407)

To a solution of 1-(tert-butyl) 2-methyl 3-formyl-1H-pyrrole-1,2-dicarboxylate 2406 (4.3 g, 0.017 mol) in DCM (100 mL) at room temperature was added ZnBr₂ (7.65 g, 0.034 mol). The reaction mixture was stirred at rt for 18 h under N₂. The reaction solution was quenched with water and extracted with EtOAc (50 mL×3). The organic phase was concentrated under reduced pressure. The residue was purified by silica gel column (eluting with PE/EtOAc=90:10 to 60:40) to obtain methyl 3-formyl-1H-pyrrole-2-carboxylate 2407 (1.2 g, 90% purity, 24% yield) as yellow oil.

LCMS (ESI) calcd for C₇H₇NO₃ [M+H]⁺ m/z 154.04, found 154.10.

### Preparation of methyl 4-bromo-3-formyl-1H-pyrrole-2-carboxylate (2408)

To a solution of methyl 3-formyl-1H-pyrrole-2-carboxylate 2407 (320 mg, 2.1 mmol) in THE (12 mL) was added NBS (409 mg, 2.3 mmol) in portions at 0° C. The reaction was stirred at 0° C. for 2 h. Water was added to quench the reaction. The obtained solution was extracted with EtOAc (50 mL×3). The combined organic phase was washed with brine, dried over Na₂SO₄ and concentrated under reduced pressure. The residue was purified by flash chromatography (eluting with PE/EtOAc=100:0 to 85:15) to give methyl 4-bromo-3-formyl-1H-pyrrole-2-carboxylate 2408 (260 mg, 90% purity, 48% yield) as a white solid.

LCMS (ESI) calcd for C₇H₆BrNO₃ [M+H]⁺ m/z 231.95, found 232.00.

### Preparation of 3-bromo-6-(4-methoxybenzyl)-1,6-dihydro-7H-pyrrolo[2,3-d]pyridazin-7-one (2410)

To a solution of methyl 4-bromo-3-formyl-1H-pyrrole-2-carboxylate 2408 (310 mg, 1.34 mmol) in AcOH (2 mL) at room temperature was added (4-methoxybenzyl)hydrazine hydrochloride 2409 (504 mg, 2.67 mmol). The reaction mixture was stirred at 80° C. for 18 h. The reaction solution was quenched with water and extracted with EtOAc (50 mL×3). The organic phase was concentrated under reduced pressure. The residue was purified by silica gel column (eluting with PE/EtOAc=90:10 to 60:40) to obtain 3-bromo-6-(4-methoxybenzyl)-1,6-dihydro-7H-pyrrolo[2,3-d]pyridazin-7-one 2410 (125 mg, 90% purity, 25% yield) as yellow solid.

LCMS (ESI) calcd for C₁₄H₁₂BrN₃O₂ [M+H]⁺ m/z 334.01 found 334.10.

### Preparation of 3-bromo-1-ethyl-6-(4-methoxybenzyl)-1,6-dihydro-7H-pyrrolo[2,3-d]pyridazin-7-one (2411)

To a solution of 3-bromo-6-(4-methoxybenzyl)-1,6-dihydro-7H-pyrrolo[2,3-d]pyridazin-7-one 2410 (445 mg, 1.33 mmol) in THE (10 mL) at 0° C. was added NaH (60%, 266 mg, 6.66 mmol). The reaction mixture was stirred at 0° C. for 10 min with N₂. Then added iodoethane (2076 mg, 13.00 mmol). The reaction mixture was stirred at 70° C. for 5 h with N. After cooling to rt, the reaction solution was quenched with ice water and extracted with EtOAc (50 mL×3). The organic phase was concentrated and purified by silica gel column to obtain 3-bromo-1-ethyl-6-(4-methoxybenzyl)-1,6-dihydro-7H-pyrrolo[2,3-d]pyridazin-7-one 2411 (410 mg, 1.02 mmol, 76% yield) as yellow oil.

LCMS (ESI) calcd for C₁₆H₁₆BrN₃O₂[M+H]⁺ m/z 362.04, found 362.00.

### Preparation of 3-allyl-1-ethyl-6-(4-methoxybenzyl)-1,6-dihydro-7H-pyrrolo[2,3-d]pyridazin-7-one (2413)

To a solution of 3-bromo-1-ethyl-6-(4-methoxybenzyl)-1,6-dihydro-7H-pyrrolo[2,3-d]pyridazin-7-one 2411 (300 mg, 0.83 mmol) in ACN (15 mL) at room temperature were added allyltributylstannane 2412 (822 mg, 2.48 mmol) and Pd(AMPHOS)Cl₂ (117 mg, 0.17 mmol). The reaction mixture was stirred at 100° C. for 3 h under N₂ in a sealed tube. After cooling to rt, the resulting reaction mixture was poured into cold saturated aqueous NH₄Cl and stirred for 5 min. Then the mixture was extracted with EtOAc (20 mL×3). The combined organic layer was washed with brine (10 mL×3), dried over Na₂SO₄ and concentrated under reduced pressure. The residue was purified by flash silica chromatography (eluting with PE/EtOAc=100:0 to 55:45) to give 3-allyl-1-ethyl-6-(4-methoxybenzyl)-1,6-dihydro-7H-pyrrolo[2,3-d]pyridazin-7-one 2413 (300 mg, 70% purity, 78% yield) as a yellow oil.

LCMS (ESI) calcd for C₁₉H₂₁N₃O₂ [M+H]⁺ m/z 324.16 found 324.20.

### Preparation of 2-(1-ethyl-6-(4-methoxybenzyl)-7-oxo-6,7-dihydro-1H-pyrrolo[2,3-d]pyridazin-3-yl)acetaldehyde (2414)

To a solution of 3-allyl-1-ethyl-6-(4-methoxybenzyl)-1,6-dihydro-7H-pyrrolo[2,3-d]pyridazin-7-one 2413 (150 mg, 0.46 mmol) in Dioxane/H₂O=3:1 (10 mL) at 0° C. were added NaIO₄ (397 mg, 1.85 mmol) and K₂OsO₄·2H₂O (17 mg, 0.046 mmol). The reaction mixture was stirred at 0° C. for 15 min. The reaction mixture was stirred at rt for 2 h. The reaction solution was quenched with water and extracted with EtOAc (20 mL×3). The organic phase was concentrated under reduced pressure to give 2-(1-ethyl-6-(4-methoxybenzyl)-7-oxo-6,7-dihydro-1H-pyrrolo[2,3-d]pyridazin-3-yl)acetaldehyde 2414 (100 mg, 70% purity, 45% yield) as a yellow oil.

LCMS (ESI) calcd for C₁₈H₁₉N₃O₃ [M+H]⁺ m/z 326.14 found 326.15.

### Preparation of 1-ethyl-3-(2-hydroxyethyl)-6-(4-methoxybenzyl)-1,6-dihydro-7H-pyrrolo[2,3-d]pyridazin-7-one (2415)

To a solution of 2-(1-ethyl-6-(4-methoxybenzyl)-7-oxo-6,7-dihydro-1H-pyrrolo[2,3-d]pyridazin-3-yl)acetaldehyde 2414 (100 mg, 0.30 mmol) in MeOH (5 mL) at 0° C. was added NaBH₄ (46 mg, 1.23 mmol). The reaction mixture was stirred at 0° C. for 1 h. The reaction solution was quenched with water and extracted with EtOAc (10 mL×3). The organic phase was concentrated under reduced pressure and the residue was purified by flash chromatography (eluting with PE/EtOAc=100:0 to 70:30) to give 1-ethyl-3-(2-hydroxyethyl)-6-(4-methoxybenzyl)-1,6-dihydro-7H-pyrrolo[2,3-d]pyridazin-7-one 2415 (70 mg, 40% purity, 27% yield) as yellow oil.

LCMS (ESI) calcd for C₁₈H₂₁N₃O₃ [M+H]⁺ m/z 328.16 found 328.15.

### Preparation of (E)-1-ethyl-6-(4-methoxybenzyl)-3-(2-((3-oxo-3-(4-(5-(trifluoromethyl)pyrimidin-2-yl)piperazin-1-yl)prop-1-en-1-yl)oxy)ethyl)-1,6-dihydro-7H-pyrrolo[2,3-d]pyridazin-7-one (2416)

To a solution of 1-ethyl-3-(2-hydroxyethyl)-6-(4-methoxybenzyl)-1,6-dihydro-7H-pyrrolo[2,3-d]pyridazin-7-one 2415 (70 mg, 0.21 mmol) in DCM (5 mL) at rt were added 1-(4-(5-(trifluoromethyl)pyrimidin-2-yl)piperazin-1-yl)prop-2-yn-1-one (79 mg, 0.28 mmol) and P(n-Bu)₃ (22 mg, 0.11 mmol). The reaction mixture was stirred at rt for 1 h. The reaction solution was quenched with water and extracted with DCM (10 mL×3). The organic phase was concentrated under reduced pressure and the residue was purified by flash chromatography (eluting with DCM/MeOH=100:0 to 95:5) to give (E)-1-ethyl-6-(4-methoxybenzyl)-3-(2-((3-oxo-3-(4-(5-(trifluoromethyl)pyrimidin-2-yl)piperazin-1-yl)prop-1-en-1-yl)oxy)ethyl)-1,6-dihydro-7H-pyrrolo[2,3-d]pyridazin-7-one 2416 (70 mg, 50% purity, 26% yield) as yellow oil.

LCMS (ESI) calcd for C₃₀H₃₂F₃N₇O₄[M+H]⁺ m/z 612.25 found 612.20.

### Preparation of 1-ethyl-6-(4-methoxybenzyl)-3-(2-(3-oxo-3-(4-(5-(trifluoromethyl)pyrimidin-2-yl)piperazin-1-yl)propoxy)ethyl)-1,6-dihydro-7H-pyrrolo[2,3-d]pyridazin-7-one (2417)

To a solution of (E)-1-ethyl-6-(4-methoxybenzyl)-3-(2-((3-oxo-3-(4-(5-(trifluoromethyl)pyrimidin-2-yl)piperazin-1-yl)prop-1-en-1-yl)oxy)ethyl)-1,6-dihydro-7H-pyrrolo[2,3-d]pyridazin-7-one 2416 (70 mg, 0.057 mmol) in MeOH (10 mL) at rt was added Pd/C (24 mg). The reaction mixture was stirred at rt for 24 h under H₂. The reaction solution was filtered and washed with MeOH (10 mL×3). The filtrate was concentrated under reduced pressure and the residue was purified by flash chromatography (eluting with DCM/MeOH=100:0 to 95:5) to give 1-ethyl-6-(4-methoxybenzyl)-3-(2-(3-oxo-3-(4-(5-(trifluoromethyl)pyrimidin-2-yl)piperazin-1-yl)propoxy)ethyl)-1,6-dihydro-7H-pyrrolo[2,3-d]pyridazin-7-one 2417 (30 mg, 70% purity, 29% yield) as yellow oil.

LCMS (ESI) calcd for C₃₀H₃₄F₃N₇O₄[M+H]⁺ m/z 614.26 found 614.20.

### Preparation of 1-ethyl-3-(2-(3-oxo-3-(4-(5-(trifluoromethyl)pyrimidin-2-yl)piperazin-1-yl)propoxy)ethyl)-1,6-dihydro-7H-pyrrolo[2,3-d]pyridazin-7-one (203)

To a solution of 1-ethyl-6-(4-methoxybenzyl)-3-(2-(3-oxo-3-(4-(5-(trifluoromethyl)pyrimidin-2-yl)piperazin-1-yl)propoxy)ethyl)-1,6-dihydro-7H-pyrrolo[2,3-d]pyridazin-7-one 2417 (30 mg, 0.04 mmol) in TFA (3 mL) at rt was added TfOH (30 mg, 0.20 mmol). The reaction mixture was stirred at rt for 1 h. The reaction solution was quenched with water and adjusted PH 7˜8 with aq. Na₂CO₃ at 0° C. The water phase was concentrated and purified on a Biotage Isolera One (C₁₈ column, eluting with 20% to 50% MeCN/H₂O containing 0.1% formic acid) and prep-TLC (DCM/MeOH=25:1) to give 1-ethyl-3-(2-(3-oxo-3-(4-(5-(trifluoromethyl)pyrimidin-2-yl)piperazin-1-yl)propoxy)ethyl)-1,6-dihydro-7H-pyrrolo[2,3-d]pyridazin-7-one 203 (2.8 mg, 93% purity, 13% yield) as a yellow solid.

¹H NMR (400 MHz, DMSO-d₆, ppm) δ: 12.24 (s, 1H), 8.73 (s, 2H), 8.19 (s, 1H), 7.37 (s, 1H), 4.44 (q, J=7.2 Hz, 2H), 3.80 (d, J=14.0 Hz, 4H), 3.68 (t, J=6.6 Hz, 2H), 3.61 (t, J=6.8 Hz, 2H), 3.58-3.52 (m, 4H), 2.85 (t, J=6.6 Hz, 2H), 2.63 (t, J=6.4 Hz, 2H), 1.34 (t, J=7.0 Hz, 3H).

LCMS (ESI) calcd for C₂₂H₂₆F₃N₇O₃[M+H]⁺ m/z 494.20, found 494.25.

### 16. Synthesis of 1-(2-(3-(4-(5-fluorobenzo[d]oxazol-2-yl)piperazin-1-yl)-3-oxopropoxy)ethyl)-3-(trifluoromethyl)-1,5-dihydro-4H-pyrazolo[3,4-d]pyridazin-4-one (compound 215)

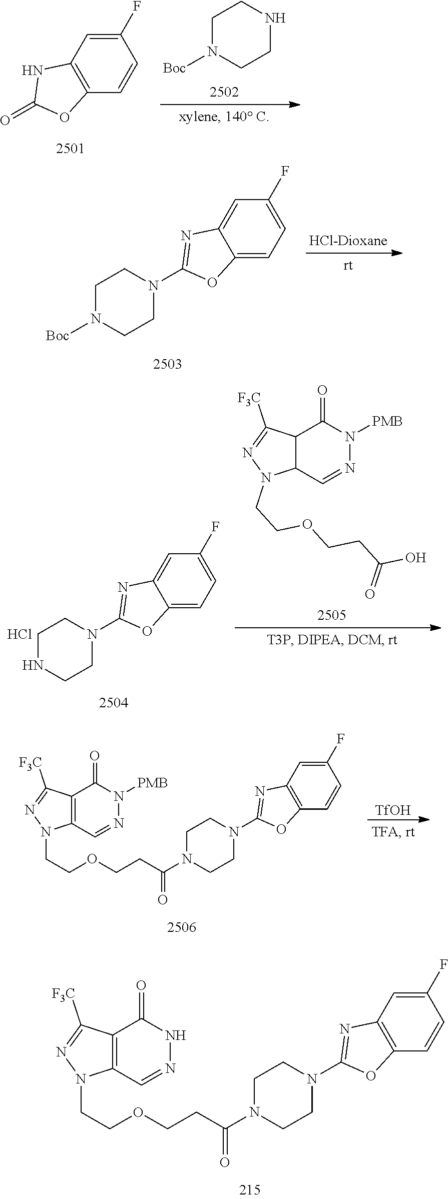

### Preparation of tert-butyl 4-(5-fluorobenzo[d]oxazol-2-yl)piperazine-1-carboxylate (2503)

Tert-butyl piperazine-1-carboxylate 2502 (5.54 g, 0.030 mol) was added to a solution of 5-fluorobenzo[d]oxazole-2(3H)-thione 2501 (2.5 g, 0.015 mol) in xylene (50 mL). The reaction mixture was stirred at 140° C. for 2 h and then concentrated under reduced pressure. The residue was purified by flash silica chromatography (eluting with EtOAc/PE, 0 to 37%) to give tert-butyl 4-(5-fluorobenzo[d]oxazol-2-yl)piperazine-1-carboxylate 2503 (4.42 g, 90% purity, 83% yield) as a white solid.

LCMS (ESI) calcd for C₁₆H₂₀FN₃O₃[M+H]⁺ m/z 322.16, found 322.20.

### Preparation of 5-fluoro-2-(piperazin-1-yl)benzo[d]oxazole hydrochloride (2504)

HCl-Dioxane (4 mol/L, 50 mL) was added to a round-bottom flask containing tert-butyl 4-(5-fluorobenzo[d]oxazol-2-yl)piperazine-1-carboxylate 2503 (4.42 g, 0.013 mol). The reaction mixture was stirred at rt for 20 min and then concentrated under reduced pressure. The residue was triturated with DCM (30 mL). The precipitate was collected and dried in vacuo to give 5-fluoro-2-(piperazin-1-yl)benzo[d]oxazole hydrochloride 2504 (3.41 g, 90% purity, 86% yield) as a white solid.

LCMS (ESI) calcd for C₁₁H₁₂FN₃O [M+H]⁺ m/z 222.10, found 222.20.

### Preparation of 1-(2-(3-(4-(5-fluorobenzo[d]oxazol-2-yl)piperazin-1-yl)-3-oxopropoxy)ethyl)-5-(4-methoxybenzyl)-3-(trifluoromethyl)-1,5-dihydro-4H-pyrazolo[3,4-d]pyridazin-4-one (2506)

DIPEA (147 mg, 1.135 mmol), 5-fluoro-2-(piperazin-1-yl)benzo[d]oxazole hydrochloride 2504 (70 mg, 0.272 mmol) and T3P (289 mg, 0.454 mmol, 50% in EtOAc) were added to a solution of 3-(2-(5-(4-methoxybenzyl)-4-oxo-3-(trifluoromethyl)-4,5-dihydro-1H-pyrazolo[3,4-d]pyridazin-1-yl)ethoxy)propanoic acid 2505 (100 mg, 0.227 mmol) in DCM (10 mL). The mixture was stirred at rt for 30 min, washed with water (20 mL) and extracted with DCM (10 mL×3). The combined organic layers were washed with brine, dried over Na₂SO₄ and concentrated under reduced pressure. The residue was purified by flash silica chromatography (eluting with EtOAc/PE, 0 to 100%) to give 1-(2-(3-(4-(5-fluorobenzo[d]oxazol-2-yl)piperazin-1-yl)-3-oxopropoxy)ethyl)-5-(4-methoxybenzyl)-3-(trifluoromethyl)-1,5-dihydro-4H-pyrazolo[3,4-d]pyridazin-4-one 2506 (150 mg, 90% purity, 92% yield) as a colorless oil.

LCMS (ESI) calcd for C₃₀H₂₉F₄N₇O₅[M+H]⁺ m/z 644.22, found 644.15.

### Preparation of 1-(2-(3-(4-(5-fluorobenzo[d]oxazol-2-yl)piperazin-1-yl)-3-oxopropoxy)ethyl)-3-(trifluoromethyl)-1,5-dihydro-4H-pyrazolo[3,4-d]pyridazin-4-one (compound 215)

TfOH (0.2 mL) was added to a solution of 1-(2-(3-(4-(5-fluorobenzo[d]oxazol-2-yl)piperazin-1-yl)-3-oxopropoxy)ethyl)-5-(4-methoxybenzyl)-3-(trifluoromethyl)-1,5-dihydro-4H-pyrazolo[3,4-d]pyridazin-4-one 2506 (140 mg, 0.217 mmol) in TFA (5 mL) at rt. The mixture was stirred at rt for 10 min. Then pH of the resulting mixture was adjusted to around 8.0 by progressively adding saturated NaHCO₃ solution under 0° C. The resulting mixture was extracted with DCM (20 mL×3). The combined organic layers were washed with brine, dried over Na₂SO₄ and concentrated under reduced pressure. The residue was purified on a Biotage Isolera One (C₁₈ column, eluting with 30% to 40% MeCN/H₂O containing 0.1% formic acid) to give 1-(2-(3-(4-(5-fluorobenzo[d]oxazol-2-yl)piperazin-1-yl)-3-oxopropoxy)ethyl)-3-(trifluoromethyl)-1,5-dihydro-4H-pyrazolo[3,4-d]pyridazin-4-one 215 (54.7 mg, 99% purity, 47% yield) as a white solid.

¹H NMR (400 MHz, DMSO-d₆, ppm) δ: 12.87 (s, 1H), 8.59 (s, 1H), 8.46 (s, 0.2H), 7.44-7.39 (m, 1H), 7.15 (dd, J=9.2, 2.4 Hz, 1H), 6.87-6.81 (m, 1H), 4.70 (t, J=4.8 Hz, 2H), 3.82 (t, J=5.0 Hz, 2H), 3.62 (t, J=6.2 Hz, 2H), 3.58-3.54 (m, 2H), 3.54-3.48 (m, 6H), 2.49-2.47 (m, 2H).

LCMS (ESI) calcd for C₂₂H₂₁F₄N₇O₄[M+H]⁺ m/z 524.17, found 524.25.

### 17. Synthesis of 5-(2,2,2-trifluoro-1-(4-oxo-4-(4-(5-(trifluoromethyl)pyrimidin-2-yl)piperazin-1-yl)butoxy)ethyl)phthalazin-1(2H)-one (compounds 221 and 222)

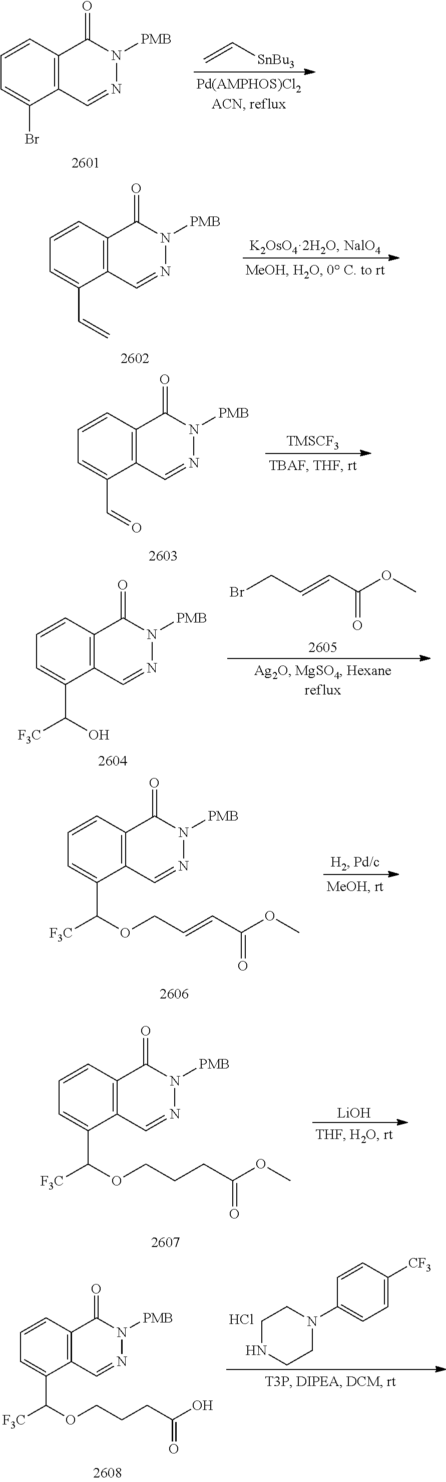

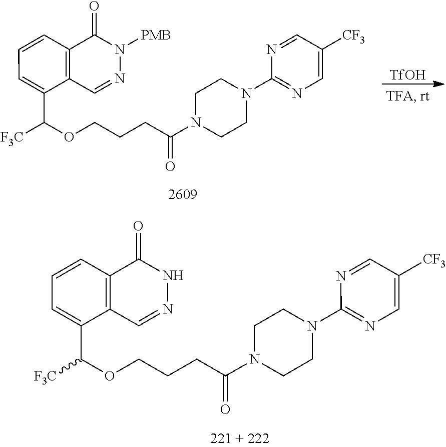

### Preparation of 2-(4-methoxybenzyl)-5-vinylphthalazin-1(2H)-one (2602)

Tributyl(vinyl)stannane (2.73 g, 0.0086 mol) and Pd(AMPHOS)Cl₂ (0.15 g, 0.0002 mol) were added to a solution of 5-bromo-2-(4-methoxybenzyl)phthalazin-1(2H)-one 2601 (1.50 g, 0.0043 mol) in MeCN (50 mL). The mixture was heated at reflux for 1 h. After cooling to rt, the mixture was concentrated under reduced pressure and purified by flash silica chromatography (eluting with EtOAc/PE, 0 to 15%) to give 2-(4-methoxybenzyl)-5-vinylphthalazin-1(2H)-one 2602 (1.01 g, 74% purity, 60% yield) as a light yellow solid.

LCMS (ESI) calcd for C₁₈H₁₆N₂O₂ [M+H]+m/z 293.13, found 293.16

### Preparation of 2-(4-methoxybenzyl)-1-oxo-1,2-dihydrophthalazine-5-carbaldehyde (2603)

K₂OsO₄·2H₂O (0.13 g, 0.0003 mol) was added to a solution of 2-(4-methoxybenzyl)-5-vinylphthalazin-1(2H)-one 2602 (1.0 g, 0.0034 mol) in MeOH/H₂O (3/1, 120 mL) at 0° C. After stirring at 0° C. for 10 min to the mixture was added NaIO₄ (2.91 g, 0.0136 mol) and then stirred for 2 h at rt. The mixture was filtered and the filtrate was extracted with EtOAc (30 mL×3). The combined organic layers were washed with brine, dried over Na₂SO₄ and concentrated under reduced pressure. The residue was purified by flash silica chromatography (eluting with EtOAc/PE, 0 to 56%) to give 2-(4-methoxybenzyl)-1-oxo-1,2-dihydrophthalazine-5-carbaldehyde 2603 (0.43 g, 90% purity, 38% yield) as a white solid.

LCMS (ESI) calcd for C₁₇H₁₄N₂O₃ [M+H]⁺ m/z 295.11, found 295.13

### Preparation of 2-(4-methoxybenzyl)-5-(2,2,2-trifluoro-1-hydroxyethyl)phthalazin-1(2H)-one (2604)

TBAF (0.1 mL, 0.139 mmol, 1 mol/L in THF) and TMSCF₃ (297 mg, 2.090 mmol) were added to a solution of 2-(4-methoxybenzyl)-1-oxo-1,2-dihydrophthalazine-5-carbaldehyde 2603 (410 mg, 1.393 mmol) in THE (20 mL). The mixture was stirred at rt for 30 min, acidified with HCl (1 mol/L) at 0° C., and extracted with EtOAc (10 mL×3). The combined organic layers were washed with brine, dried over Na₂SO₄ and concentrated under reduced pressure. The residue was purified with flash silica chromatography (eluting with EtOAc/PE, 0 to 19%) to give 2-(4-methoxybenzyl)-5-(2,2,2-trifluoro-1-hydroxyethyl)phthalazin-1(2H)-one 2604 (300 mg, 90% purity, 53% yield) as a white solid.

LCMS (ESI) calcd for C₁₈H₁₅F₃N₂O₃[M+H]⁺ m/z 365.11, found 365.00

### Preparation of methyl (E)-4-(2,2,2-trifluoro-1-(2-(4-methoxybenzyl)-1-oxo-1,2-dihydrophthalazin-5-yl)ethoxy)but-2-enoate (2606)

Ag₂O (951 mg, 4.106 mmol) and MgSO₄ (493 mg, 4.106 mmol) were added to a solution of 2-(4-methoxybenzyl)-5-(2,2,2-trifluoro-1-hydroxyethyl)phthalazin-1(2H)-one 2604 (300 mg, 0.821 mmol) in hexane (40 mL). The mixture was heated at reflux for 1 h, then methyl (E)-4-bromobut-2-enoate 2605 (294 mg, 1.642 mmol) was added. The mixture was heated at reflux for 16 h. After cooling to rt, the mixture was filtered to remove Ag₂O and the filtrate was concentrated under reduced pressure. The residue was purified by flash silica chromatography (eluting with EtOAc/PE, 0 to 35%) to give methyl (E)-4-(2,2,2-trifluoro-1-(2-(4-methoxybenzyl)-1-oxo-1,2-dihydrophthalazin-5-yl)ethoxy)but-2-enoate 2606 (270 mg, 70% purity, 50% yield) as a light yellow oil.

LCMS (ESI) calcd for C₂₃H₂₁F₃N₂O₅[M+Na]⁺ m/z 485.11, found 485.22

### Preparation of methyl 4-(2,2,2-trifluoro-1-(2-(4-methoxybenzyl)-1-oxo-1,2-dihydrophthalazin-5-yl)ethoxy)butanoate (2607)

To a solution of methyl (E)-4-(2,2,2-trifluoro-1-(2-(4-methoxybenzyl)-1-oxo-1,2-dihydrophthalazin-5-yl)ethoxy)but-2-enoate 2606 (260 mg, 0.561 mmol) in MeOH (20 mL) was added Pd/C (10%, 50 mg) at rt. The suspension was degassed with H₂ for 6 times. The reaction mixture was stirred at rt for 3 h under H₂ atmosphere. The resulting reaction mixture was filtered through celite. The filtrate was concentrated under reduced pressure to obtain methyl 4-(2,2,2-trifluoro-1-(2-(4-methoxybenzyl)-1-oxo-1,2-dihydrophthalazin-5-yl)ethoxy)butanoate 2607 (240 mg, 59% purity, 54% yield) as a yellow oil.

LCMS (ESI) calcd C₂₃H₂₃F₃N₂O₅[M+H]⁺ m/z 465.16, found 465.19

### Preparation of 4-(2,2,2-trifluoro-1-(2-(4-methoxybenzyl)-1-oxo-1,2-dihydrophthalazin-5-yl)ethoxy)butanoic acid (2608)

LiOH (35 mg, 1.483 mmol) was added to a solution of methyl 4-(2,2,2-trifluoro-1-(2-(4-methoxybenzyl)-1-oxo-1,2-dihydrophthalazin-5-yl)ethoxy)butanoate 2607 (230 mg, 0.494 mmol) in THF/H₂O (3/1, 16 mL). The mixture was stirred at rt for 2 h, concentrated under reduced pressure to remove THF, acidified with HCl solution (1 mol/L) and extracted with DCM (10 mL×3). The combined organic layers were washed with brine, dried over Na₂SO₄ and concentrated in vacuo to give 4-(2,2,2-trifluoro-1-(2-(4-methoxybenzyl)-1-oxo-1,2-dihydrophthalazin-5-yl)ethoxy)butanoic acid 2608 (240 mg, 60% purity, 64% yield) as a light yellow oil.

LCMS (ESI) calcd for C₂₂H₂₁F₃N₂O₅[M+H]⁺ m/z 451.15, found 451.15

### Preparation of 2-(4-methoxybenzyl)-5-(2,2,2-trifluoro-1-(4-oxo-4-(4-(5-(trifluoromethyl)pyrimidin-2-yl)piperazin-1-yl)butoxy)ethyl)phthalazin-1(2H)-one (2609)

DIPEA (329 mg, 2.547 mmol), 2-(piperazin-1-yl)-5-(trifluoromethyl)pyrimidine hydrochloride (164 mg, 0.611 mmol) and T3P (648 mg, 1.019 mmol, 50% in EtOAc) were added to a solution of 4-(2,2,2-trifluoro-1-(2-(4-methoxybenzyl)-1-oxo-1,2-dihydrophthalazin-5-yl)ethoxy)butanoic acid 2608 (230 mg, 0.509 mmol) in DCM (10 mL). The mixture was stirred at rt for 30 min, diluted with water (20 mL) and extracted with DCM (20 mL×3). The combined organic layers were washed with brine, dried over Na₂SO₄ and concentrated under reduced pressure to give 2-(4-methoxybenzyl)-5-(2,2,2-trifluoro-1-(4-oxo-4-(4-(5-(trifluoromethyl)pyrimidin-2-yl)piperazin-1-yl)butoxy)ethyl)phthalazin-1(2)-one 2609 (300 mg, 70% purity, 62% yield) as a light yellow oil.

LCMS (ESI) calcd for C₃₁H₃₀F₆N₆O₄[M+H]⁺ m/z 665.23, found 665.15

### Preparation of 5-(2,2,2-trifluoro-1-(4-oxo-4-(4-(5-(trifluoromethyl)pyrimidin-2-yl)piperazin-1-yl)butoxy)ethyl)phthalazin-1(2H)-one (compounds 221 and 222)

TfOH (0.2 mL) was added to a solution of 2-(4-methoxybenzyl)-5-(2,2,2-trifluoro-1-(4-oxo-4-(4-(5-(trifluoromethyl)pyrimidin-2-yl)piperazin-1-yl)butoxy)ethyl)phthalazin-1(2H)-one 2609 (300 mg, 0.451 mmol) in TFA (10 mL). The reaction mixture was stirred at rt for 10 min, adjusted to pH=8 by progressively adding saturated NaHCO₃ solution then extracted with DCM (20 mL×3). The combined organic layers were washed with brine, dried over Na₂SO₄ and concentrated under reduced pressure. The residue was purified with C₁₈ column (Agela 40 g, mobile phase: MeCN—H₂O (0.1% FA), gradient: 40%-50%) to give 5-(2,2,2-trifluoro-1-(4-oxo-4-(4-(5-(trifluoromethyl)pyrimidin-2-yl)piperazin-1-yl)butoxy)ethyl)phthalazin-1(2H)-one 221 and 222 (93 mg, 99%, 37% yield) as a white solid.

### Chiral resolution of 5-(2,2,2-trifluoro-1-(4-oxo-4-(4-(5-(trifluoromethyl)pyrimidin-2-yl)piperazin-1-yl)butoxy)ethyl)phthalazin-1(2H)-one (compounds 221 and 222)

Compounds 221 and 222 were separated by SFC (Column: Daicel CHIRALPAK IC 250 mm×20 mm I.D., 5 m; Mobile phase: CO₂/MeOH (0.1% NH₃)=65/35) and concentrated under reduced pressure to afford the first fraction as 221 (24.7 mg, 99% purity, 100% ee, white solid) and the second fraction as 222 (32.7 mg, 99% purity, 100% ee, white solid).

### Compound 221

¹H NMR (400 MHz, DMSO-d₆, ppm) δ: 12.85 (s, 1H), 8.77 (s, 1H), 8.74 (s, 2H), 8.35 (d, J=8.0 Hz, 1H), 8.10 (d, J=7.6 Hz, 1H), 7.95 (t, J=7.8 Hz, 1H), 6.04-5.95 (m, 1H), 3.86-3.81 (m, 2H), 3.80-3.74 (m, 2H), 3.72-3.65 (m, 1H), 3.58-3.48 (m, 5H), 2.46-2.35 (m, 2H), 1.88-1.79 (m, 2H).

LCMS (ESI) calcd for C₂₃H₂₂F₆N₆O₃[M+H]⁺ m/z 545.17, found 545.15

### Compound 222

¹H NMR (400 MHz, DMSO-d₆, ppm) δ. 12.85 (s, 1H), 8.77 (s, 1H), 8.74 (s, 2H), 8.35 (d, J=8.0 Hz, 1H), 8.10 (d, J=7.6 Hz, 1H), 7.95 (t, J=7.8 Hz, 1H), 6.05-5.97 (m, 1H), 3.86-3.80 (m, 2H), 3.80-3.75 (m, 2H), 3.72-3.65 (m, 1H), 3.59-3.48 (m, 5H), 2.46-2.35 (m, 2H), 1.89-1.79 (m, 2H).

LCMS (ESI) calcd for C₂₃H₂₂F₆N₆O₃[M+H]⁺ m/z 545.17, found 545.10

### 18. Synthesis of 1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrrolo[2,3-d]pyridazin-4-one (compounds 223 and 224)

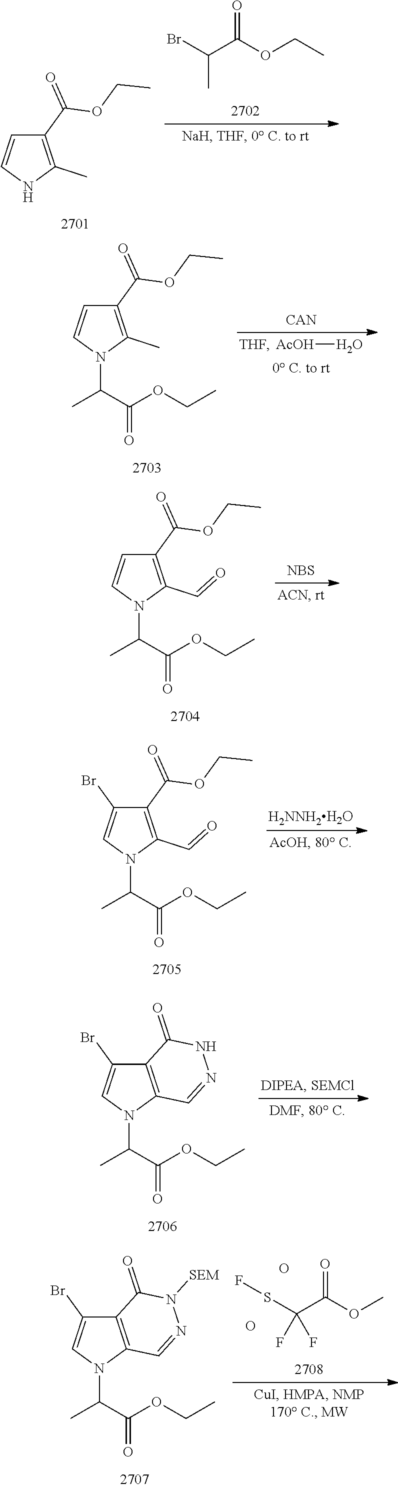

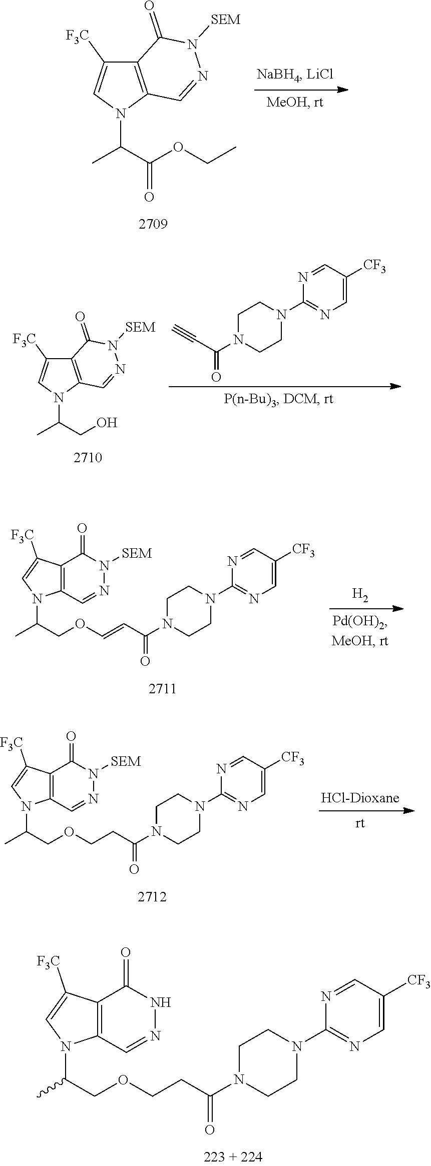

### Preparation of ethyl 1-(1-ethoxy-1-oxopropan-2-yl)-2-methyl-1H-pyrrole-3-carboxylate (2703)

To a stirred solution of NaH (60% wt, 5.95 g, 148.8 mmol) in THF (400 mL) at 0° C. was added ethyl 2-methyl-1H-pyrrole-3-carboxylate 2701 (4.06 g, 26.5 mmol) in portions. After stirring 15 min at 0° C., ethyl 2-bromopropanoate 2702 (24.4 g, 134.8 mmol) was added dropwise and the reaction was warmed to rt and stirred for 16 h. The reaction was quenched with saturated aqueous NH₄Cl and extracted with EtOAc (200 mL×4). The combined organic layers were washed with brine (30 mL), dried over Na₂SO₄ and concentrated in vacuo. The residue was purified by flash chromatography (eluting with PE/EtOAc=100:0 to 95:5) to afford ethyl 1-(1-ethoxy-1-oxopropan-2-yl)-2-methyl-1H-pyrrole-3-carboxylate 2703 (7.38 g, 98% purity, 72% yield) as an off-white solid.

LCMS (ESI) calcd for C₁₃H₁₉NO₄ [M+H]⁺ m/z 254.13, found 254.10.

### Preparation of ethyl 1-(1-ethoxy-1-oxopropan-2-yl)-2-formyl-1H-pyrrole-3-carboxylate (2704)

Ethyl 1-(1-ethoxy-1-oxopropan-2-yl)-2-methyl-1H-pyrrole-3-carboxylate 2703 (7.38 g, 28.88 mmol) was dissolved in THE (885 mL) under stirring, followed by addition a solution of AcOH (148 mL) and H₂O (148 mL). The mixture was homogeneously stirred at 0° C. and then CAN (94.4 g, 172.5 mmol) was added in one portion. After stirring at rt for 1 h, the reaction mixture was poured into ice-water (2000 mL) and stirred for another 30 min. The resulting solution was extracted with EtOAc (500 mL×3). The combined organic layer was washed with brine, dried over Na₂SO₄ and concentrated under reduced pressure. The residue was purified by flash chromatography (eluting with PE/EtOAc=100:0 to 90:10) to obtain the title compound ethyl 1-(1-ethoxy-1-oxopropan-2-yl)-2-formyl-1H-pyrrole-3-carboxylate 2704 (9.09 g, 90% purity, 86% yield) as a white solid.

LCMS (ESI) calcd for C₁₃H₁₇NO₅ [M+H]⁺ m/z 268.11, found 268.15.

### Preparation of ethyl 4-bromo-1-(1-ethoxy-1-oxopropan-2-yl)-2-formyl-1H-pyrrole-3-carboxylate (2705)

To a solution of ethyl 1-(1-ethoxy-1-oxopropan-2-yl)-2-formyl-1H-pyrrole-3-carboxylate 2704 (9.09 g, 33.9 mmol) in ACN (500 mL) was added NBS (5.8 g, 32.5 mmol) in one portion. The reaction mixture was stirred at rt for 1 h. The resulting mixture was diluted with water and extracted with DCM (30 mL×3). The combined organic layer was dried over Na₂SO₄ and concentrated under reduced pressure. The residue was purified by flash chromatography (eluting with PE/EtOAc=100:0 to 70:30) to obtain ethyl 4-bromo-1-(1-ethoxy-1-oxopropan-2-yl)-2-formyl-1H-pyrrole-3-carboxylate 2705 (3.23 g, 90% purity, 26% yield) as a white solid.

¹H NMR (400 MHz, DMSO-d₆, ppm) δ 10.01 (s, 1H), 7.74 (s, 1H), 5.63 (q, J=7.2 Hz, 1H), 4.33 (q, J=7.2 Hz, 2H), 4.15-4.10 (m, 2H), 1.67 (d, J=7.6 Hz, 3H), 1.32 (t, J=7.0 Hz, 3H), 1.17 (t, J=7.0 Hz, 3H)

### Preparation of ethyl 2-(3-bromo-4-oxo-4,5-dihydro-1H-pyrrolo[2,3-d]pyridazin-1-yl)propanoate (2706)

To a solution of ethyl 4-bromo-1-(1-ethoxy-1-oxopropan-2-yl)-2-formyl-1H-pyrrole-3-carboxylate 2705 (3.23 g, 9.37 mmol) in AcOH (50 mL) was added H₂NNH₂·H₂O (80% wt, 630 mg, 15.75 mmol) in one portion. The reaction mixture was heated with stirring at 80° C. for 2 h. The solvent was removed by evaporation (55° C.) under reduced pressure. The residue was diluted with DCM (50 mL) and then adjusted to pH to 8 with saturated aqueous NaHCO₃ at 0° C. The basified solution was extracted with DCM (10 mL×3). The combined organic layer was washed with brine, dried over Na₂SO₄ and concentrated under reduced pressure to obtain ethyl 2-(3-bromo-4-oxo-4,5-dihydro-1H-pyrrolo[2,3-d]pyridazin-1-yl)propanoate 2706 (2.06 g, 95% purity, 67% yield) as a white solid.

LCMS (ESI) calcd for C₁₁H₁₂BrN₃O₃ [M+H]⁺ m/z 314.01, found 314.05

### Preparation of ethyl 2-(3-bromo-4-oxo-5-((2-(trimethylsilyl)ethoxy)methyl)-4,5-dihydro-1H-pyrrolo[2,3-d]pyridazin-1-yl)propanoate (2707)

To a solution of ethyl 2-(3-bromo-4-oxo-4,5-dihydro-1H-pyrrolo[2,3-d]pyridazin-1-yl)propanoate 2706 (2.06 g, 6.6 mmol) and DIPEA (4.3 g, 33.3 mmol) in DMF (100 mL) at rt, SEMCl (5.40 g, 32.5 mmol) was added. After completing of addition, the reaction solution was heated at 80° C. for 1 h. The resulting reaction solution was cooled and poured into cold water, and then extracted with EtOAc (200 mL×3). The combined organic layer was washed with brine, dried over Na₂SO₄ and concentrated under reduced pressure. The residue was purified by flash chromatography (eluting with PE/EtOAc=70:30 to 40:60) to give ethyl 2-(3-bromo-4-oxo-5-((2-(trimethylsilyl)ethoxy)methyl)-4,5-dihydro-1H-pyrrolo[2,3-d]pyridazin-1-yl)propanoate 2707 (3 g, 90% purity, 89% yield) as a white solid.

LCMS (ESI) calcd for C₁₇H₂₆BrN₃O₄Si [M+H]⁺ m/z 444.09, found 444.05.

### Preparation of ethyl 2-(4-oxo-3-(trifluoromethyl)-5-((2-(trimethylsilyl)ethoxy)methyl)-4,5-dihydro-1H-pyrrolo[2,3-d]pyridazin-1-yl)propanoate (2709)

A solution of ethyl 2-(3-bromo-4-oxo-5-((2-(trimethylsilyl)ethoxy)methyl)-4,5-dihydro-1H-pyrrolo[2,3-d]pyridazin-1-yl)propanoate 2707 (2.24 g, 5.0 mmol) and methyl 2,2-difluoro-2-(fluorosulfonyl)acetate 2708 (4.86 g, 25.2 mmol), CuI (1.92 g, 10.1 mmol) and HMPA (4.51 g, 25.2 mmol) in NMP (30 mL) was prepared at rt. The mixture was heated at 170° C. in a microwave reactor for 1.5 hours under an atmosphere of N₂. The resulting reaction solution was poured into cold water and then extracted with EtOAc (200 mL×3). The combined organic layer was washed with brine, dried over Na₂SO₄ and concentrated under reduced pressure. The residue was purified by flash chromatography (eluting with EtOAc/PE=0%-30%) to give ethyl 2-(4-oxo-3-(trifluoromethyl)-5-((2-(trimethylsilyl)ethoxy)methyl)-4,5-dihydro-1H-pyrrolo[2,3-d]pyridazin-1-yl)propanoate 2709 (0.665 g, 65% purity, 33% yield) as a yellow solid.

LCMS (ESI) calcd for C₁₈H₂₆F₃N₃O₄Si [M+H]⁺ m/z 434.16, found 434.10.

### Preparation of 1-(1-hydroxypropan-2-yl)-3-(trifluoromethyl)-5-((2-(trimethylsilyl)ethoxy)methyl)-1,5-dihydro-4H-pyrrolo[2,3-d]pyridazin-4-one (2710)

To a suspension of ethyl 2-(4-oxo-3-(trifluoromethyl)-5-((2-(trimethylsilyl)ethoxy)methyl)-4,5-dihydro-1H-pyrrolo[2,3-d]pyridazin-1-yl)propanoate 2709 (665 mg, 1.53 mmol) and LiCl (265 mg, 6.25 mmol) in MeOH (20 mL) was added NaBH₄ (243 mg, 6.42 mmol) at 0° C. Then the reaction mixture was stirred at rt for 2 h. The resulting mixture was quenched with saturated aqueous NH₄Cl and then extracted with EtOAc. The combined organic layer was washed with brine, dried over Na₂SO₄ and concentrated under reduced pressure. The residue was purified by flash chromatography (eluting with DCM/MeOH=100:0 to 90:10) to obtain 1-(1-hydroxypropan-2-yl)-3-(trifluoromethyl)-5-((2-(trimethylsilyl)ethoxy)methyl)-1,5-dihydro-4H-pyrrolo[2,3-d]pyridazin-4-one 2710 (365 mg, 65% purity, 73% yield) as a yellow oil.

LCMS (ESI) calcd for C₁₆H₂₄F₃N₃O₃Si [M+H]⁺ m/z 392.15, found 392.17.

### Preparation of (E)-1-(1-((3-oxo-3-(4-(5-(trifluoromethyl)pyrimidin-2-yl)piperazin-1-yl)prop-1-en-1-yl)oxy)propan-2-yl)-3-(trifluoromethyl)-5-((2-(trimethylsilyl)ethoxy)methyl)-1,5-dihydro-4H-pyrrolo[2,3-d]pyridazin-4-one(2711)

A round-bottom flask containing a mixture of 1-(1-hydroxypropan-2-yl)-3-(trifluoromethyl)-5-((2-(trimethylsilyl)ethoxy)methyl)-1,5-dihydro-4H-pyrrolo[2,3-d]pyridazin-4-one 2710 (130 mg, 0.332 mmol), 1-(4-(5-(trifluoromethyl)pyrimidin-2-yl)piperazin-1-yl)prop-2-yn-1-one (124 mg, 0.437 mmol) and P(n-Bu)₃ (68 mg, 0.337 mmol) in DCM (20 mL) was stirred at rt for 16 h. The resulting reaction solution was concentrated under reduced pressure. The residue was purified by flash chromatography (eluting with DCM/MeOH=90:10) to give of (E)-1-(1-((3-oxo-3-(4-(5-(trifluoromethyl)pyrimidin-2-yl)piperazin-1-yl)prop-1-en-1-yl)oxy)propan-2-yl)-3-(trifluoromethyl)-5-((2-(trimethylsilyl)ethoxy)methyl)-1,5-dihydro-4H-pyrrolo[2,3-d]pyridazin-4-one 2711 (156 mg, 70% purity, 69% yield) as a yellow solid.

LCMS (ESI) calcd for C₂₈H₃₅F₆N₇O₄Si [M+H]⁺ m/z 676.24, found 676.20.

### Preparation 1-(1-(3-oxo-3-(4-(5-(trifluoromethyl)pyrimidin-2-yl)piperazin-1-yl)propoxy)propan-2-yl)-3-(trifluoromethyl)-5-((2-(trimethylsilyl)ethoxy)methyl)-1,5-dihydro-4H-pyrrolo[2,3-d]pyridazin-4-one (2712)

A mixture of compound (E)-1-(1-((3-oxo-3-(4-(5-(trifluoromethyl)pyrimidin-2-yl)piperazin-1-yl)prop-1-en-1-yl)oxy)propan-2-yl)-3-(trifluoromethyl)-5-((2-(trimethylsilyl)ethoxy)methyl)-1,5-dihydro-4H-pyrrolo[2,3-d]pyridazin-4-one 2711 (156 mg, 0.231 mmol) and Pd(OH)₂ (150 mg) in MeOH (20 mL) was stirred at rt under H₂ atmosphere for 16 hours. The resulting mixture was filtered through diatomaceous earth. The filtrate was concentrated to dryness under reduced pressure. The residue was purified by flash chromatography (eluting with DCM/MeOH=98:2) to 1-(1-(3-oxo-3-(4-(5-(trifluoromethyl)pyrimidin-2-yl)piperazin-1-yl)propoxy)propan-2-yl)-3-(trifluoromethyl)-5-((2-(trimethylsilyl)ethoxy)methyl)-1,5-dihydro-4H-pyrrolo[2,3-d]pyridazin-4-one 2712 (83 mg, 80% purity, 70% yield) as a white solid.

LCMS (ESI) calcd for C₂₈H₃₇F₆N₇O₄Si [M+H]⁺ m/z 678.26, found 678.30.

### Preparation of 1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrrolo[2,3-d]pyridazin-4-one (223 and 224)

A round-bottom flask containing a mixture of 1-(1-(3-oxo-3-(4-(5-(trifluoromethyl)pyrimidin-2-yl)piperazin-1-yl)propoxy)propan-2-yl)-3-(trifluoromethyl)-5-((2-(trimethylsilyl)ethoxy)methyl)-1,5-dihydro-4H-pyrrolo[2,3-d]pyridazin-4-one 2712 (83 mg, 0.123 mmol), HCl in 1,4-dioxane (4 M, 15 mL) was stirred at rt for 12 h. The resulting mixture was concentrated to dryness under reduced pressure. The mixture was adjusted to pH 8-9 with saturated aq. NaHCO₃, and then extracted with EtOAc (50 mL×3). The combined organic layers were concentrated under reduced pressure. The residue was purified by C₁₈ column (Agela 40 g, mobile phase: ACN-H₂O (0.1% FA), gradient: 10%-95%) to give 1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrrolo[2,3-d]pyridazin-4-one 223 and 224 (54 mg, 88% purity, 77% yield) as a white solid.

LCMS (ESI) calcd for C₂₂H₂₃F₆N₇O₃[M+H]⁺ m/z 548.18, found 548.27.

### Chiral resolution of 1-(1-(3-oxo-3-(4-(5-(trifluoromethyl)pyrimidin-2-yl)piperazin-1-yl)propoxy)propan-2-yl)-3-(trifluoromethyl)-1,5-dihydro-4H-pyrrolo[2,3-d]pyridazin-4-one (223 and 224)

Compounds 223 and 224 were separated by SFC (Column: DAICEL AD-H 4.6 mm I.D.×250 mmL 5 um; Mobile phase: CO₂/MeOH (0.1% NH₃)=70/30) and concentrated under reduced pressure to afford the first fraction as 223 (14.0 mg, 99% purity, 100% ee, off-white solid) and the second fraction as 224 (15.2 mg, 99% purity, 93% ee, white solid).

### Compound 223

¹H NMR (400 MHz, DMSO-d₆, ppm) δ: 12.55 (s, 1H), 8.73 (s, 2H), 8.49 (s, 1H), 8.14 (s, 1H), 4.99-4.88 (m, 1H), 3.82-3.70 (m, 6H), 3.70-3.54 (m, 2H), 3.52-3.42 (m, 4H), 2.49-2.45 (m, 2H), 1.46 (d, J=6.8 Hz, 3H).

LCMS (ESI) calcd for C₂₂H₂₃F₆N₇O₃ [M+H]⁺ m/z 548.18, found 548.15.

### Compound 224

¹H NMR (400 MHz, DMSO-d₆, ppm) δ: 12.55 (s, 1H), 8.73 (s, 2H), 8.49 (s, 1H), 8.14 (s, 1H), 5.00-4.87 (m, 1H), 3.81-3.70 (m, 6H), 3.70-3.54 (m, 2H), 3.52-3.42 (m, 4H), 1.46 (d, J=7.2 Hz, 3H).

LCMS (ESI) calcd for C₂₂H₂₃F₆N₇O₃ [M+H]⁺ m/z 548.18, found 548.15.

### 19. Synthesis of 3-(2-(3-oxo-3-(4-(5-(trifluoromethyl)pyrimidin-2-yl)piperazin-1-yl)propoxy)ethyl)-1-(trifluoromethyl)-6,7-dihydroimidazo[1,5-a]pyrazin-8(5H)-one (compound 235)

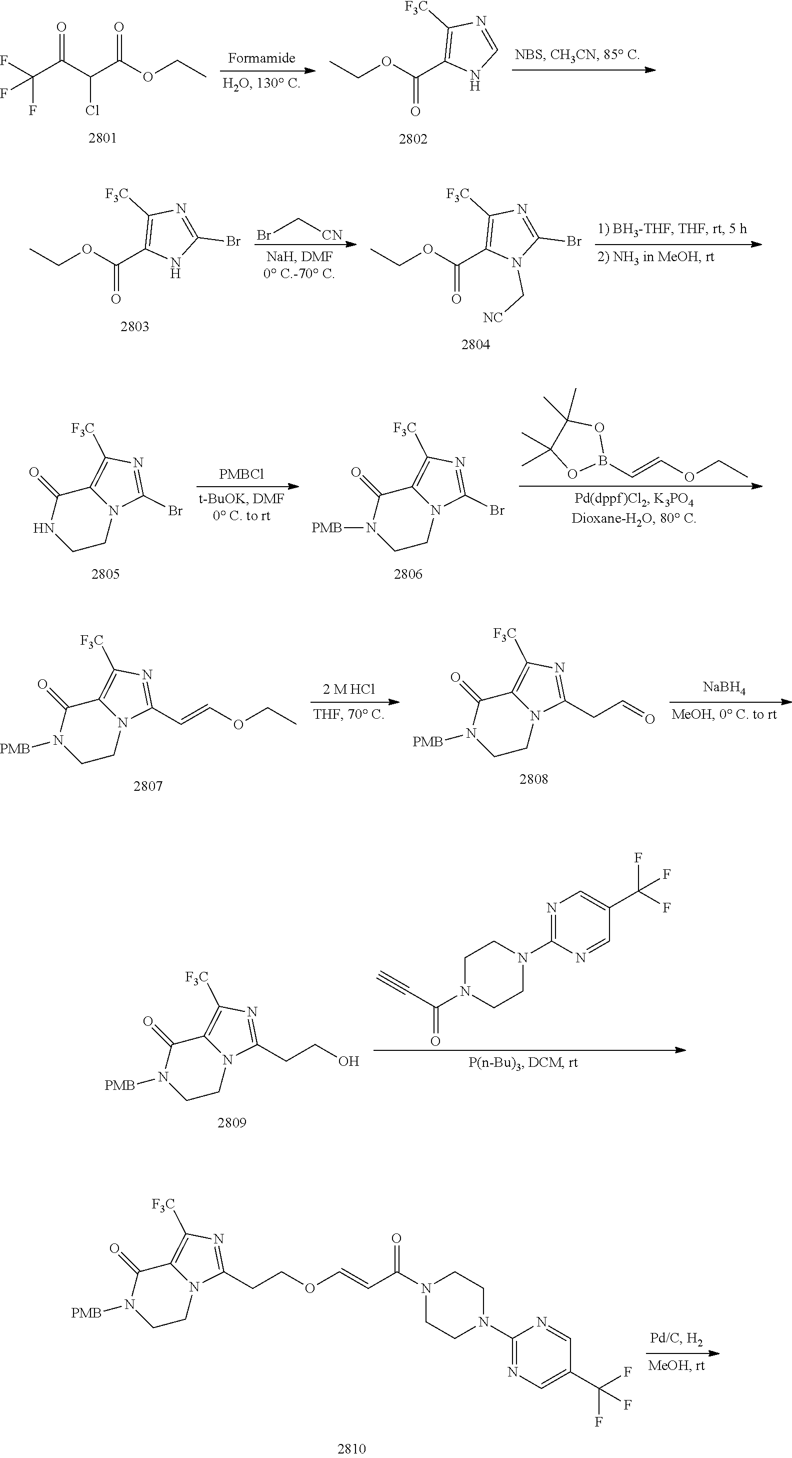

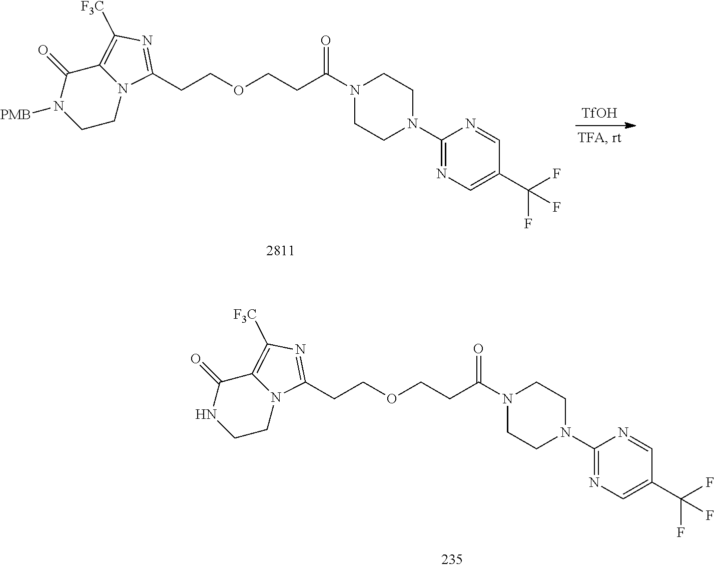

### Preparation of ethyl 4-(trifluoromethyl)-1H-imidazole-5-carboxylate (2802)

Ethyl 2-chloro-4,4,4-trifluoro-3-oxobutanoate 2801 (2180 mg, 10 mmol) was combined with formamide (4492 mg, 100 mmol) and water (0.4 mL). The reaction was heated at 130° C. for 1.5 h. The mixture was then cooled to rt, and 8 mL of ice water was added. The resulting solids were collected and washed with water then dried in vacuo to give ethyl 4-(trifluoromethyl)-1H-imidazole-5-carboxylate 2802 (440 mg, 90% purity, 19% yield) as a brown solid.

LCMS (ESI) calcd for C₇H₇F₃N₂O₂ [M+H]⁺ m/z 209.05, found 209.10

### Preparation of ethyl 2-bromo-4-(trifluoromethyl)-1H-imidazole-5-carboxylate (2803)

To a solution of ethyl 4-(trifluoromethyl)-1H-imidazole-5-carboxylate 2802 (1500 mg, 7.2 mmol) in CH₃CN (15 mL) was added NBS (1530 mg, 8.4 mmol). The mixture was stirred at 85° C. for 2 h. Water was added to quench the reaction. The obtained solution was extracted with EtOAc (40 mL×4). The combined organic phase was washed with brine, dried over Na₂SO₄ and concentrated under reduced pressure. The residue was purified by flash chromatography (eluting with PE/EtOAc=100:0 to 95:5) to give ethyl 2-bromo-4-(trifluoromethyl)-1H-imidazole-5-carboxylate 2803 (1560 mg, 90% purity, 67% yield) as a white solid.

LCMS (ESI) calcd for C₇H₆BrF₃N₂O₂ [M+H]⁺ m/z 286.95, found 287.00

### Preparation of ethyl 2-bromo-1-(cyanomethyl)-4-(trifluoromethyl)-1H-imidazole-5-carboxylate (2804)

To a solution of NaH (60%, 600 mg, 15 mmol) in DMF (5 mL) was added dropwise a solution of ethyl 2-bromo-4-(trifluoromethyl)-1H-imidazole-5-carboxylate 2803 (1440 mg, 5 mmol) and 2-bromoacetonitrile (600 mg, 5 mmol) in DMF (20 mL) at 0° C. under N₂ atmosphere. The reaction mixture was warmed to 70° C. and kept stirring for an additional 2 h. After completion of reaction, the reaction mixture was cooled to rt and then poured into cold saturated aqueous NH₄Cl. The resulting solution was extracted with EtOAc (40 mL×3). The combined organic layer was washed with brine, dried over Na₂SO₄ and concentrated under reduced pressure. The residue was purified by flash silica chromatography (eluting with PE/EtOAc=100:0 to 90:10) to give ethyl 2-bromo-1-(cyanomethyl)-4-(trifluoromethyl)-1H-imidazole-5-carboxylate 2804 (1250 mg, 90% purity, 68.8 yield) as a white solid.

LCMS (ESI) calcd for C₉H₇BrF₃N₃O₂ [M+H]⁺ m/z 325.97, found 326.05

### Preparation of 3-bromo-1-(trifluoromethyl)-6,7-dihydroimidazo[1,5-a]pyrazin-8(5H)-one (2805)

To a solution of ethyl 2-bromo-3-(cyanomethyl)-5-(trifluoromethyl)imidazole-4-carboxylate 2804 (1280 mg, 4 mmol) in THF (40 mL) was added slowly BH₃-THF (1 M, 20 mL, 20 mmol) at 0° C. After completion of addition, the reaction solution was warmed to rt and kept stirring at rt for an additional 5 h. The resulting reaction solution was added dropwise MeOH (15 mL) to quench the BH₃-THF at rt (caution: gas released) and then concentrated under reduced pressure to get ethyl 1-(2-aminoethyl)-2-bromo-4-(trifluoromethyl)-1H-imidazole-5-carboxylate (600 mg, 85% purity, 40% yield) as a white oil.

LCMS (ESI) calcd for C₉H₁₁BrF₃N₃O₂ [M+H]⁺ m/z 330.0, found 330.05

Ethyl 1-(2-aminoethyl)-2-bromo-4-(trifluoromethyl)-1H-imidazole-5-carboxylate (1200 mg, 3.6 mmol) was diluted with NH₃-MeOH (7 M, 7 mL) and kept stirring at rt overnight. LCMS monitored the formation of product, the reaction mixture was evaporated under reduced pressure to afford 3-bromo-1-(trifluoromethyl)-6,7-dihydroimidazo[1,5-a]pyrazin-8(5H)-one 2805 (700 mg, 75% purity, 51% yield) as an white solid

LCMS (ESI) calcd for C₇H₅BrF₃N₃O [M+H]⁺ m/z 283.96, found 284.00

### Preparation of 3-bromo-7-(4-methoxybenzyl)-1-(trifluoromethyl)-6,7-dihydroimidazo[1,5-a]pyrazin-8(5H)-one (2806)

To a solution of t-BuOK (664 mg, 6 mmol) in DMF (12 mL) was added slowly a solution of 3-bromo-1-(trifluoromethyl)-6,7-dihydroimidazo[1,5-a]pyrazin-8(5H)-one 2805 (560 mg, 2 mmol) and PMBCl (926 mg, 6 mmol) in DMF (8 mL) at 0° C. under N₂ atmosphere. After completion of addition, the reaction was warmed to rt and kept stirring at rt for an additional 1.5 h. The resulting reaction mixture was poured into cold saturated aqueous NH₄Cl and stirred for 5 min. Then the solution was extracted with EtOAc (50 mL×3). The combined organic layer was washed with brine (50 mL×3), dried over Na₂SO₄ and concentrated under reduced pressure. The residue was purified by flash silica chromatography (eluting with PE/EtOAc=100:0 to 55:45) to give 3-bromo-7-(4-methoxybenzyl)-1-(trifluoromethyl)-6,7-dihydroimidazo[1,5-a]pyrazin-8(5H)-one 2806 (600 mg, 90% purity, 67% yield) as a yellow solid.

LCMS (ESI) calcd for C₁₅H₁₃BrF₃N₃O₂ [M+H]⁺ m/z 404.01, found 404.10

### Preparation of (E)-3-(2-ethoxyvinyl)-7-(4-methoxybenzyl)-1-(trifluoromethyl)-6,7-dihydroimidazo[1,5-a]pyrazin-8(5H)-one (2807)

A solution of 3-bromo-7-(4-methoxybenzyl)-1-(trifluoromethyl)-6,7-dihydroimidazo[1,5-a]pyrazin-8(5H)-one 2806 (480 mg, 1.2 mmol), (E)-2-(2-ethoxyvinyl)-4,4,5,5-tetramethyl-1,3,2-dioxaborolane (471 mg, 2.4 mmol), K₃PO₄ (278 mg, 1.2 mmol) and Pd(dppf)Cl₂ (87 mg, 0.12 mmol) in Dioxane/H₂O (30 mL, 3:1) was stirred at 80° C. for 4 h under N₂ atmosphere. After cooling to rt, the reaction mixture was added into cold water and then extracted with EtOAc (60 mL×3). The combined organic layer was washed with brine, dried over Na₂SO₄ and concentrated under reduced pressure. The residue was purified by flash silica chromatography (eluting with PE/EtOAc=100:0 to 50:50) to give (E)-3-(2-ethoxyvinyl)-7-(4-methoxybenzyl)-1-(trifluoromethyl)-6,7-dihydroimidazo[1,5-a]pyrazin-8(5H)-one 2807 (480 mg, 70% purity, 74% yield) as a colorless oil.

LCMS (ESI) calcd for C₁₉H₂₀F₃N₃O₃[M+H]⁺ m/z 396.15, found 396.0

### Preparation of 2-(7-(4-methoxybenzyl)-8-oxo-1-(trifluoromethyl)-5,6,7,8-tetrahydroimidazo[1,5-a]pyrazin-3-yl)acetaldehyde (2808)

To a solution of (E)-3-(2-ethoxyvinyl)-7-(4-methoxybenzyl)-1-(trifluoromethyl)-6,7-dihydroimidazo[1,5-a]pyrazin-8(5H)-one 2807 (390 mg, 1 mmol) in THE (30 mL) was added 2 M aqueous HCl (15 mL) in one portion. The solution was then stirred at 70° C. for 5 h. The resulting reaction solution was basified (pH 8) by saturated aqueous NaHCO₃ at 0° C. and then extracted with EtOAc (30 mL×3). The combined organic layer was washed with brine, dried over Na₂SO₄ and concentrated under reduced pressure. The crude 2-(7-(4-methoxybenzyl)-8-oxo-1-(trifluoromethyl)-5,6,7,8-tetrahydroimidazo[1,5-a]pyrazin-3-yl)acetaldehyde 2808 (200 mg crude, 80% purity, 44.3% yield) as a brown oil was used directly in next step.

LCMS (ESI) calcd for C₁₇H₁₆F₃N₃O₃[M+H]⁺ m/z 368.11, found 368.20

### Preparation of 3-(2-hydroxyethyl)-7-(4-methoxybenzyl)-1-(trifluoromethyl)-6,7-dihydroimidazo[1,5-a]pyrazin-8(5H)-one (2809)

To a solution of 2-(7-(4-methoxybenzyl)-8-oxo-1-(trifluoromethyl)-5,6,7,8-tetrahydroimidazo[1,5-a]pyrazin-3-yl)acetaldehyde 2808 (900 mg, 2.5 mmol) in MeOH (10 mL) was added NaBH₄ (140 mg, 3.6 mmol) in portions at 0° C. The solution was warmed to rt and stirred for 1 h. Saturated aqueous NH₄Cl (15 mL) was added into the reaction solution and then stirred for 5 min. The resulting solution was extracted with EtOAc (50 mL×4). The combined organic layer was washed with brine, dried over Na₂SO₄ and concentrated under reduced pressure. The residue was purified by flash silica chromatography (eluting with PE/EtOAc=50:50 to 0:100) to give 3-(2-hydroxyethyl)-7-(4-methoxybenzyl)-1-(trifluoromethyl)-6,7-dihydroimidazo[1,5-a]pyrazin-8(5H)-one 2809 (620 mg, 90% purity, 62% yield) as a yellow solid.

LCMS (ESI) calcd for C₁₇H₁₈F₃N₃O₃[M+H]⁺ m/z 370.13, found 370.15

### Preparation of (E)-7-(4-methoxybenzyl)-3-(2-((3-oxo-3-(4-(5-(trifluoromethyl)pyrimidin-2-yl)piperazin-1-yl)prop-1-en-1-yl)oxy)ethyl)-1-(trifluoromethyl)-6,7-dihydroimidazo[1,5-a]pyrazin-8(5H)-one (2810)

To a solution of 3-(2-hydroxyethyl)-7-(4-methoxybenzyl)-1-(trifluoromethyl)-6,7-dihydroimidazo[1,5-a]pyrazin-8(5H)-one 2809 (200 mg, 0.56 mmol) and 1-(4-(5-(trifluoromethyl)pyrimidin-2-yl)piperazin-1-yl)prop-2-yn-1-one (168 mg, 0.60 mmol) in DCM (20 mL) was added P(n-Bu)₃ (56 mg, 0.28 mmol) dropwise. After completion of addition, the reaction solution was stirred at rt for 2 h. The resulting solution was evaporated under reduced pressure to get a deep brown solid crude which was purified by flash silica chromatography (eluting with PE/EtOAc=50:50 to 0:100) to give (E)-7-(4-methoxybenzyl)-3-(2-((3-oxo-3-(4-(5-(trifluoromethyl)pyrimidin-2-yl)piperazin-1-yl)prop-1-en-1-yl)oxy)ethyl)-1-(trifluoromethyl)-6,7-dihydroimidazo[1,5-a]pyrazin-8(5H)-one 2810 (228 mg, 90% purity, 58% yield) as a yellow solid.

LCMS (ESI) calcd for C₂₉H₂₉F₆N₇O₄[M+Na]⁺ m/z 676.21, found 676.75

### Preparation of 2-(4-methoxybenzyl)-6-(2-(3-oxo-3-(4-(5-(trifluoromethyl)pyrimidin-2-yl)piperazin-1-yl)propoxy)ethyl)-3,4-dihydropyrrolo[1,2-a]pyrazin-1(2H)-one (2811)

A solution of (E)-7-(4-methoxybenzyl)-3-(2-((3-oxo-3-(4-(5-(trifluoromethyl)pyrimidin-2-yl)piperazin-1-yl)prop-1-en-1-yl)oxy)ethyl)-1-(trifluoromethyl)-6,7-dihydroimidazo[1,5-a]pyrazin-8(5H)-one 2810 (280 mg, 0.43 mmol) and Pd/C (46 mg) in MeOH (10 mL) was stirred at rt for 1 h under H₂ atmosphere. The resulting solution was filtered through diatomaceous earth and the filter cake was washed with MeOH (10 mL×4). The filtrate was concentrated under reduced pressure to give 2-(4-methoxybenzyl)-6-(2-(3-oxo-3-(4-(5-(trifluoromethyl)pyrimidin-2-yl)piperazin-1-yl)propoxy)ethyl)-3,4-dihydropyrrolo[1,2-a]pyrazin-1(2H)-one 2811 (99 mg, 90% purity, 32% yield) as a colorless oil, which was used directly in the next step without further purification.

LCMS (ESI) calcd for C₂₉H₃₁F₆N₇O₄ [M+H]⁺ m/z 656.23, found 656.30

### Preparation of 3-(2-(3-oxo-3-(4-(5-(trifluoromethyl)pyrimidin-2-yl)piperazin-1-yl)propoxy)ethyl)-1-(trifluoromethyl)-6,7-dihydroimidazo[1,5-a]pyrazin-8(5H)-one (compound 235)

To a solution of 2-(4-methoxybenzyl)-6-(2-(3-oxo-3-(4-(5-(trifluoromethyl)pyrimidin-2-yl)piperazin-1-yl)propoxy)ethyl)-3,4-dihydropyrrolo[1,2-a]pyrazin-1(2H)-one 2811 (100 mg, 0.15 mmol) in TFA (10 ml) was added TfOH (228 mg, 1.5 mmol) at rt. After completion of addition, the reaction solution was stirred at rt for 3 h. The resulting solution was concentrated under reduced pressure to remove most TFA. The residue was diluted with DCM (20 mL) and then adjusted to pH to 8 with saturated aqueous NaHCO₃ at 0° C. The basified solution was extracted with DCM (20 mL×3). The combined organic layer was washed with brine, dried over Na₂SO₄ and concentrated under reduced pressure. The crude product was purified by flash silica chromatography (eluting with DCM/MeOH=100:0 to 90:10) and C18 column (Agela 40 g, mobile phase: ACN-H₂O (0.1% FA), gradient: 30-60) to give 3-(2-(3-oxo-3-(4-(5-(trifluoromethyl)pyrimidin-2-yl)piperazin-1-yl)propoxy)ethyl)-1-(trifluoromethyl)-6,7-dihydroimidazo[1,5-a]pyrazin-8(5H)-one (compound 235, 14.6 mg, 100% purity, 17% yield) as a white solid.

¹H NMR (400 MHz, DMSO-d₆, ppm) δ: 8.73 (s, 2H), 8.24 (s, 1H), 4.16 (t, J=5.8 Hz, 2H), 3.87-3.75 (m, 4H), 3.71 (t, J=6.4 Hz, 2H), 3.65 (t, J=6.4 Hz, 2H), 3.56-3.48 (m, 6H), 2.96 (t, J=6.4 Hz, 2H), 2.60 (t, J=6.4 Hz, 2H).

LCMS (ESI) calcd for C₂₁H₂₃F₆N₇O₃ [M+H]⁺ m/z 536.18, found 536.30

### 20. Synthesis of 3-(difluoromethyl)-1-(2-(3-oxo-3-(4-(5-(trifluoromethyl)pyrimidin-2-yl)piperazin-1-yl)propoxy)ethyl)-1,5-dihydro-4H-pyrazolo[3,4-d]pyridazin-4-one (compound 236)

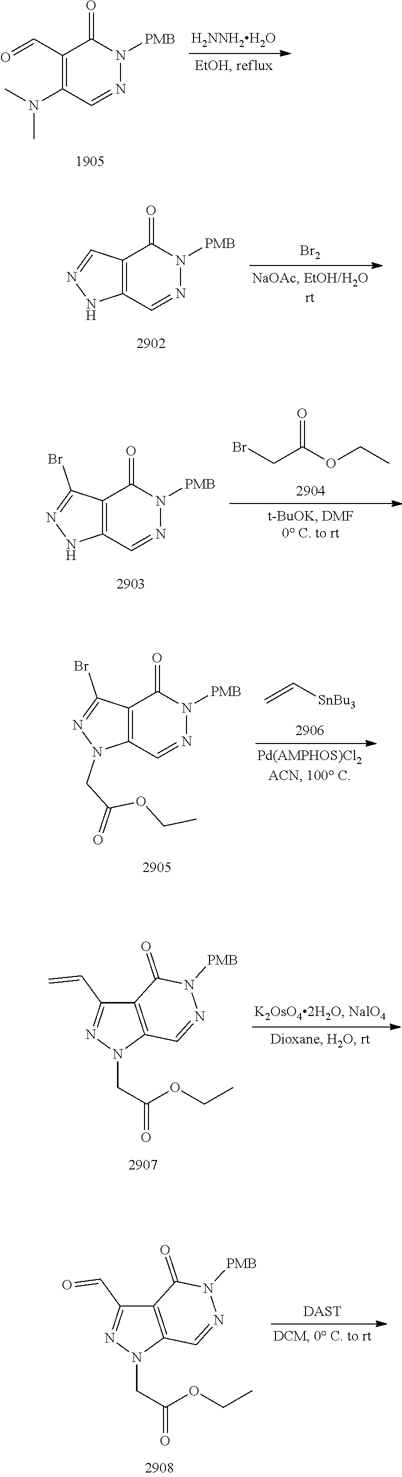

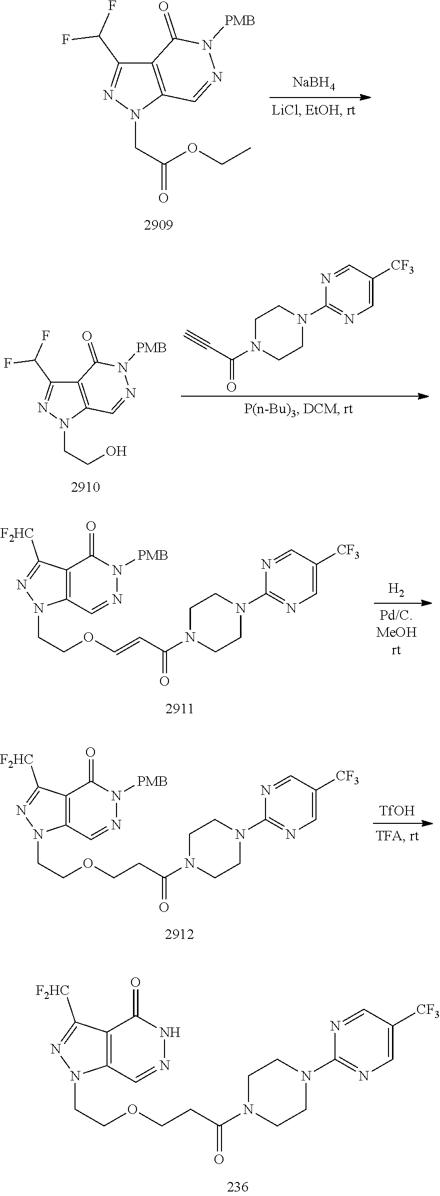

### Preparation of 5-(4-methoxybenzyl)-1,5-dihydro-4H-pyrazolo[3,4-d]pyridazin-4-one (2902)

To a solution of 5-(dimethylamino)-2-(2-methoxy-5-methylphenyl)-3-oxopyridazine-4-carbaldehyde 1905 (2.6 g, 0.009 mol) in EtOH (50 mL) was added H₂NNH₂·H₂O (80% wt, 2.0 g, 0.050 mol) at rt. The reaction mixture was stirred at 80° C. for 24 h. The resulting mixture was concentrated under reduced pressure. The residue was purified by flash column chromatography (eluting with PE/EtOAc=20:80 to 0:100) to afford 5-(4-methoxybenzyl)-1,5-dihydro-4H-pyrazolo[3,4-d]pyridazin-4-one 2902 (1.5 g, 90% purity, 59% yield) as a white solid.

LCMS (ESI) calcd for C₁₃H₂N₄O₂ [M+H]⁺ m/z 257.10, found 257.13.

### Preparation of 3-bromo-5-(4-methoxybenzyl)-1,5-dihydro-4H-pyrazolo[3,4-d]pyridazin-4-one (2903)

To a solution of 5-(2-methoxy-5-methylphenyl)-1H-pyrazolo[3,4-d]pyridazin-4-one 2902 (1.13 g, 4.4 mmol) in EtOH/H₂O (1:1, 20 mL) was added NaOAc (2.53 g, 30.8 mmol) and Br₂ (2.81 g, 17.6 mmol) successively at rt. The reaction mixture was stirred at rt for 1 h. The resulting mixture was quenched with saturated aq. Na₂S₂O₃ and extracted with EtOAc (50 mL×2). The combined organic layers were concentrated under reduced pressure. The residue was purified by flash column chromatography (eluting with PE/EtOAc=85:15 to 70:30) to afford 3-bromo-5-(4-methoxybenzyl)-1,5-dihydro-4H-pyrazolo[3,4-d]pyridazin-4-one 2903 (1.08 g, 90% purity, 66% yield) as a yellow solid.

LCMS (ESI) calcd for C₁₃H₁₁BrN₄O₂[M+H]⁺ m/z 335.01, found 335.10.

### Preparation of ethyl 2-(3-bromo-5-(4-methoxybenzyl)-4-oxo-4,5-dihydro-1H-pyrazolo[3,4-d]pyridazin-1-yl)acetate (2905)

To a solution of 3-bromo-5-(4-methoxybenzyl)-1,5-dihydro-4H-pyrazolo[3,4-d]pyridazin-4-one 2903 (1.0 g, 3.0 mmol) in DMF (20 mL) was added t-BuOK (1.01 g, 9.0 mmol) at 0° C. The reaction mixture was stirred at 0° C. for 15 min and ethyl 2-bromoacetate 2904 (1.5 g, 9.0 mmol) was added dropwise at 0° C. The reaction solution was stirred at rt for 2 h. The reaction solution was quenched with ice-water and extracted with EtOAc (20 mL×3). The organic phase was concentrated and purified by flash column chromatography (eluting with PE/EtOAc=85:15 to 50:50) to obtain ethyl 2-(3-bromo-5-(4-methoxybenzyl)-4-oxo-4,5-dihydro-1H-pyrazolo[3,4-d]pyridazin-1-yl)acetate 2905 (1.3 g, 90% purity, 97% yield) as a white solid.

LCMS (ESI) calcd for C₁₇H₁₇BrN₄O₄[M+H]⁺ m/z 421.04, found 421.15.

### Preparation of ethyl 2-(5-(4-methoxybenzyl)-4-oxo-3-vinyl-4,5-dihydro-1H-pyrazolo[3,4-d]pyridazin-1-yl)acetate (2907)

To a solution of ethyl 2-(3-bromo-5-(4-methoxybenzyl)-4-oxo-4,5-dihydro-1H-pyrazolo[3,4-d]pyridazin-1-yl)acetate 2905 (1.35 g, 3.2 mmol) in ACN (30 mL) were added tributyl(vinyl) stannane 2906 (1.52 g, 4.8 mmol) and Pd(AMPHOS)Cl₂ (230 mg, 0.3 mmol) at rt. The resulting mixture was stirred at 100° C. for 1 h. After cooling to rt, the mixture was concentrated in vacuo. The residue was purified by flash chromatography (eluting with PE/EtOAc=100:0 to 40: 60) to obtain ethyl 2-(5-(4-methoxybenzyl)-4-oxo-3-vinyl-4,5-dihydro-1H-pyrazolo[3,4-d]pyridazin-1-yl)acetate 2907 (1.0 g, 90% purity, 75% yield) as a yellow solid.

LCMS (ESI) calcd for C₁₉H₂₀N₄O₄ [M+H]⁺ m/z 369.15 found 369.20.

### Preparation of ethyl 2-(3-formyl-5-(4-methoxybenzyl)-4-oxo-4,5-dihydro-1H-pyrazolo[3,4-d]pyridazin-1-yl)acetate (2908)

To a solution of ethyl 2-(5-(4-methoxybenzyl)-4-oxo-3-vinyl-4,5-dihydro-1H-pyrazolo[3,4-d]pyridazin-1-yl)acetate 2907 (770 mg, 2.09 mmol) in Dioxane/H₂O (3:1, 20 mL) were added K₂OsO₄·2H₂O (77 mg, 0.21 mmol) and NaIO₄ (1.8 g, 8.36 mol) at rt. The reaction mixture was stirred at rt for 4 h. The resulting mixture was diluted with water and extracted with EtOAc (50 mL×3). The combined organic layer was washed with brine and concentrated under reduced pressure. The residue was purified by flash column chromatography (eluting with PE/EtOAc=100:0 to 50:50) to give ethyl 2-(3-formyl-5-(4-methoxybenzyl)-4-oxo-4,5-dihydro-1H-pyrazolo[3,4-d]pyridazin-1-yl)acetate 2908 (520 mg, 90% purity, 60% yield) as a yellow oil.

LCMS (ESI) calcd for C₁₈H₁₈N₄O₅[M+H]⁺ m/z 371.13, found 371.20.

### Preparation of ethyl 2-(3-(difluoromethyl)-5-(4-methoxybenzyl)-4-oxo-4,5-dihydro-1H-pyrazolo[3,4-d]pyridazin-1-yl)acetate (2909)

To a solution of ethyl 2-(3-formyl-5-(4-methoxybenzyl)-4-oxo-4,5-dihydro-1H-pyrazolo[3,4-d]pyridazin-1-yl)acetate 2908 (0.5 g, 1.35 mmol) in DCM (20 mL) was added DAST (1.74 g, 10.8 mmol) dropwise at 0° C. under N₂ atmosphere. The reaction solution was stirred at rt for 2 h. The reaction was quenched with aq. NaHCO₃, the aqueous layer was extracted with DCM (100 mL×3). The combined organic layers were concentrated under reduced pressure. The residue was purified by flash column chromatography (eluting with PE/EtOAc=70:30 to 50:50) to afford ethyl 2-(3-(difluoromethyl)-5-(4-methoxybenzyl)-4-oxo-4,5-dihydro-1H-pyrazolo[3,4-d]pyridazin-1-yl)acetate 2909 (420 mg, 90% purity, 71% yield) as a white solid.

LCMS (ESI) calcd for C₁₈H₁₈F₂N₄O₄ [M+H]⁺ m/z 393.13, found 393.18.

### Preparation of 3-(difluoromethyl)-1-(2-hydroxyethyl)-5-(4-methoxybenzyl)-1,5-dihydro-4H-pyrazolo[3,4-d]pyridazin-4-one (2910)

To a solution of ethyl 2-(3-(difluoromethyl)-5-(4-methoxybenzyl)-4-oxo-4,5-dihydro-1H-pyrazolo[3,4-d]pyridazin-1-yl)acetate 2909 (320 mg, 0.81 mmol) in EtOH (10 mL) were added LiCl (138 mg, 3.26 mmol) and NaBH₄ (123 mg, 3.26 mmol) at rt. The reaction mixture was stirred at rt for 1 h. The resulting mixture was quenched with water and extracted with EtOAc (50 mL×3). The combined organic layers were washed with brine and concentrated under reduced pressure. The residue was purified by silica gel column chromatography (eluting with EtOAc/PE, 0 to 50%) to obtain 3-(difluoromethyl)-1-(2-hydroxyethyl)-5-(4-methoxybenzyl)-1,5-dihydro-4H-pyrazolo[3,4-d]pyridazin-4-one 2910 (270 mg, 80% purity, 75% yield) as white solid.

LCMS (ESI) calcd for C₁₆H₁₆F₂N₄O₃[M+H]⁺ m/z 351.12, found 351.20

### Preparation of (E)-3-(difluoromethyl)-5-(4-methoxybenzyl)-1-(2-((3-oxo-3-(4-(5-(trifluoromethyl)pyrimidin-2-yl)piperazin-1-yl)prop-1-en-1-yl)oxy)ethyl)-1,5-dihydro-4H-pyrazolo[3,4-d]pyridazin-4-one (2911)

To a solution of 3-(difluoromethyl)-1-(2-hydroxyethyl)-5-(4-methoxybenzyl)-1,5-dihydro-4H-pyrazolo[3,4-d]pyridazin-4-one 2910 (150 mg, 0.42 mmol), 1-(4-(5-(trifluoromethyl)pyrimidin-2-yl)piperazin-1-yl)prop-2-yn-1-one (122 mg, 0.42 mmol) in DCM (15 mL) was added P(n-Bu)₃ (43 mg, 0.21 mmol) at rt. The reaction mixture was stirred at rt for 1 h. The resulting mixture was quenched with water and extracted with DCM (50 mL×3). The combined organic layer was washed with brine and concentrated under reduced pressure. The residue was purified by silica gel column (eluting with EtOAc/PE, 0 to 100%) to obtain (E)-3-(difluoromethyl)-5-(4-methoxybenzyl)-1-(2-((3-oxo-3-(4-(5-(trifluoromethyl)pyrimidin-2-yl)piperazin-1-yl)prop-1-en-1-yl)oxy)ethyl)-1,5-dihydro-4H-pyrazolo[3,4-d]pyridazin-4-one 2911 (190 mg, 90% purity, 63% yield) as a white solid.

LCMS (ESI) calcd for C₂₈H₂₇F₅N₈O₄ [M+H]⁺ m/z 635.21, found 635.40

### Preparation of 3-(difluoromethyl)-5-(4-methoxybenzyl)-1-(2-(3-oxo-3-(4-(5-(trifluoromethyl)pyrimidin-2-yl)piperazin-1-yl)propoxy)ethyl)-1,5-dihydro-4H-pyrazolo[3,4-d]pyridazin-4-one (2912)

To a solution of (E)-3-(difluoromethyl)-5-(4-methoxybenzyl)-1-(2-((3-oxo-3-(4-(5-(trifluoromethyl)pyrimidin-2-yl)piperazin-1-yl)prop-1-en-1-yl)oxy)ethyl)-1,5-dihydro-4H-pyrazolo[3,4-d]pyridazin-4-one 2911 (100 mg, 0.15 mmol) in MeOH (10 mL) was added 10% Pd/C (10 mg). The mixture was evacuated and backfilled with hydrogen three times and then charged with hydrogen. The resulting mixture was stirred at room temperature for 2 hours under N₂. Then the mixture was filtered through celite and concentrated under vacuum to give 3-(difluoromethyl)-5-(4-methoxybenzyl)-1-(2-(3-oxo-3-(4-(5-(trifluoromethyl)pyrimidin-2-yl)piperazin-1-yl)propoxy)ethyl)-1,5-dihydro-4H-pyrazolo[3,4-d]pyridazin-4-one 2912 (100 mg, 90% purity, 90% yield) which was used directly in next step without further purification.

LCMS (ESI) calcd for C₂₈H₂₉F₅N₈O₄[M+H]⁺ m/z 637.22, found 637.35

### Preparation of 3-(difluoromethyl)-1-(2-(3-oxo-3-(4-(5-(trifluoromethyl)pyrimidin-2-yl)piperazin-1-yl)propoxy)ethyl)-1,5-dihydro-4H-pyrazolo[3,4-d]pyridazin-4-one (compound 236)

To a solution of 3-(difluoromethyl)-5-(4-methoxybenzyl)-1-(2-(3-oxo-3-(4-(5-(trifluoromethyl)pyrimidin-2-yl)piperazin-1-yl)propoxy)ethyl)-1,5-dihydro-4H-pyrazolo[3,4-d]pyridazin-4-one 2912 (100 mg, 0.15 mmol) in TFA (1 mL) was added TfOH (707 mg, 4.71 mmol) dropwise. The mixture was stirred in rt for 2 h. The resulting light brown solution was concentrated under reduced pressure to remove most TFA. The residue was diluted with DCM (10 mL) and then adjusted pH to 8 with saturated aqueous NaHCO₃ at 0° C. The basified solution was extracted with DCM (10 mL×3). The combined organic layer was washed with brine, dried over Na₂SO₄ and concentrated under reduced pressure. The crude product was purified by C₁₈ column (Agela 40 g, mobile phase: ACN-H₂O (0.1% FA), gradient: 30%-60%) to give 3-(difluoromethyl)-1-(2-(3-oxo-3-(4-(5-(trifluoromethyl)pyrimidin-2-yl)piperazin-1-yl)propoxy)ethyl)-1,5-dihydro-4H-pyrazolo[3,4-d]pyridazin-4-one 236 (39.4 mg, 100% purity, 48% yield) as a white solid.

¹H NMR (400 MHz, DMSO-d₆, ppm) δ:12.75 (s, 1H), 8.73 (s, 2H), 8.55 (s, 1H), 7.30 (t, J=53.2 Hz, 1H), 4.66 (t, J=5.0 Hz, 2H), 3.87-3.73 (m, 6H), 3.63 (t, J=6.4 Hz, 2H), 3.52-3.42 (m, 4H), 2.49-2.46 (m, 2H).

LCMS (ESI) calcd for C₂₀H₂₁F₅N₈O₃[M+H]⁺ m/z 517.17, found 517.29

### 21. Synthesis of 1-(2-(3-oxo-3-(4-(5-(trifluoromethyl)pyridin-2-yl)piperazin-1-yl)propoxy)ethyl)-4,6-dihydropyrido[2,3-d]pyridazine-2,5(1H,3H)-dione (compound 240)

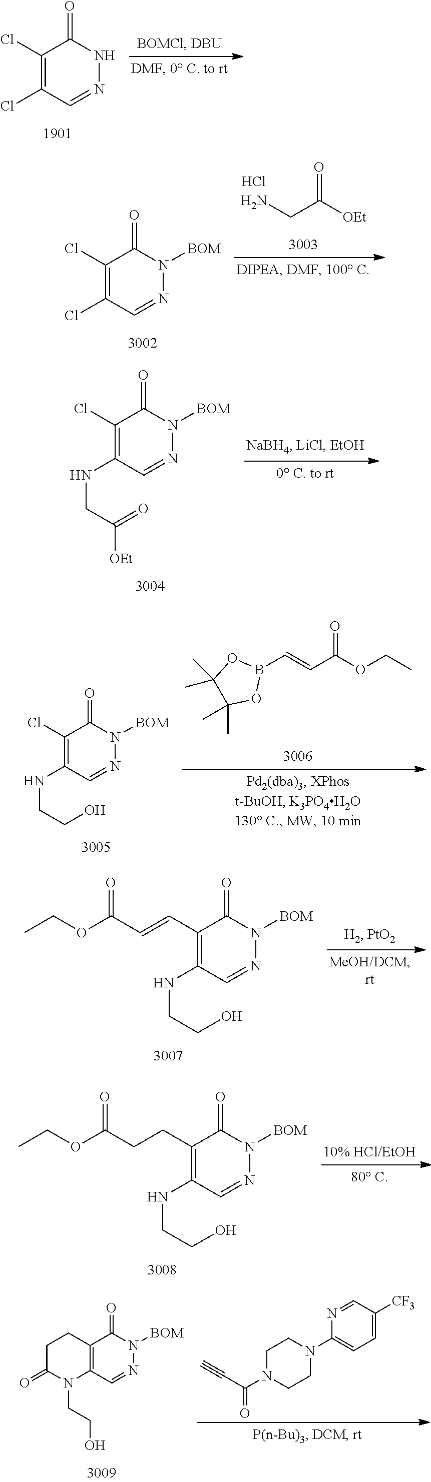

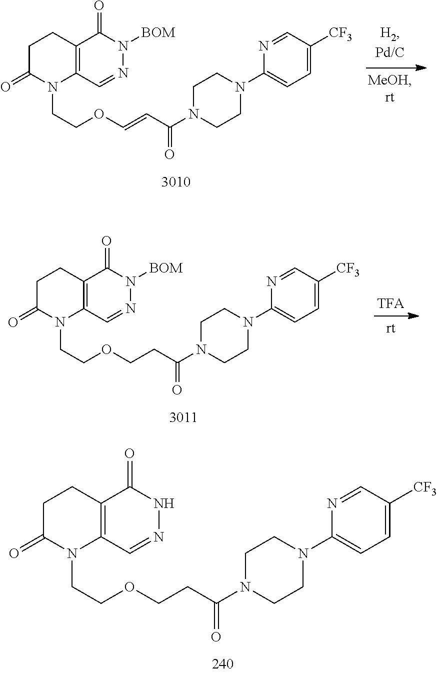

### Preparation of 2-((benzyloxy)methyl)-4,5-dichloropyridazin-3(2H)-one (3002)

To a solution of 4,5-dichloropyridazin-3(2H)-one 1901 (10 g, 0.061 mol) in DMF (100 mL) at 0° C. were added BOMCl (11.39 g, 0.073 mol) and DBU (11.07 g, 0.073 mol). The reaction mixture was stirred at rt for 2 h. The reaction solution was quenched with water and extracted with EtOAc (50 mL×3). The organic phase was concentrated under reduced pressure. The residue was purified by silica gel column (eluting with PE/EtOAc=100:0 to 95:5) to obtain 2-((benzyloxy)methyl)-4,5-dichloropyridazin-3(2H)-one 3002 (13.5 g, 90% purity, 70% yield) as a white solid.

LCMS (ESI) calcd for C₁₂H₁₀Cl₂N₂O₂ [M+H]⁺ m/z 285.01, found 284.95.

### Preparation of ethyl (1-((benzyloxy)methyl)-5-chloro-6-oxo-1,6-dihydropyridazin-4-yl)glycinate (3004)

To a solution of 2-((benzyloxy)methyl)-4,5-dichloropyridazin-3(2H)-one 3002 (13.5 g, 0.047 mol) in DMF (230 mL) at room temperature were added ethyl 2-aminoacetate HCl salt 3003 (5.37 g, 0.052 mol) and DIPEA (18.34 g, 0.14 mol). The reaction mixture was stirred at 100° C. for 3 h. The reaction solution was quenched with water and extracted with EtOAc (300 mL×3). The organic phase was concentrated under reduced pressure. The residue was purified by silica gel column (eluting with PE/EtOAc=100:0 to 70:30) to obtain ethyl (1-((benzyloxy)methyl)-5-chloro-6-oxo-1,6-dihydropyridazin-4-yl)glycinate 3004 (15 g, 90% purity, 81% yield) as yellow solid.

LCMS (ESI) calcd for C₁₆H₁ₛClN₃O₄[M+H]⁺ m/z 352.10, found 352.05.

### Preparation of 2-((benzyloxy)methyl)-4-chloro-5-((2-hydroxyethyl)amino)pyridazin-3(2H)-one (3005)

To a solution of ethyl (1-((benzyloxy)methyl)-5-chloro-6-oxo-1,6-dihydropyridazin-4-yl)glycinate 3004 (4.2 g, 0.012 mol) in EtOH (300 mL) at 0° C. was added NaBH₄ (1.80 g, 0.048 mol) and LiCl (2.02 g, 0.048 mol). The reaction mixture was stirred at rt for 1 h. The reaction solution was quenched with water and extracted with EtOAc (50 mL×3). The organic phase was concentrated under reduced pressure. The residue was purified by silica gel column (eluting with PE/EtOAc=100:0 to 0:100) to obtain 2-((benzyloxy)methyl)-4-chloro-5-((2-hydroxyethyl)amino)pyridazin-3(2H)-one 3005 (3.4 g, 90% purity, 83% yield) as yellow solid.

LCMS (ESI) calcd for C₁₄H₁₆ClN₃O₃[M+H]⁺ m/z 310.09, found 310.00.

### Preparation of ethyl (E)-3-(2-((benzyloxy)methyl)-5-((2-hydroxyethyl)amino)-3-oxo-2,3-dihydropyridazin-4-yl)acrylate (3007)

To a solution of 2-((benzyloxy)methyl)-4-chloro-5-((2-hydroxyethyl)amino)pyridazin-3(2H)-one 3005 (3.0 g, 0.0097 mol) in t-BuOH (120 mL) at room temperature were added ethyl (E)-3-(4,4,5,5-tetramethyl-1,3,2-dioxaborolan-2-yl)acrylate 3006 (2.63 g, 0.012 mol), Pd₂(dba)₃ (0.27 g, 0.0003 mol), XPhos (0.55 g, 0.0012 mol) and K₃PO₄H₂O (5.58 g, 0.024 mol). The reaction mixture was stirred at 130° C. for 10 min with N₂ under microwave. The reaction solution was quenched with water and extracted with EtOAc (100 mL×3). The organic phase was concentrated under reduced pressure. The residue was purified by silica gel column (eluting with PE/EtOAc=90:10 to 0:100) to obtain ethyl (E)-3-(2-((benzyloxy)methyl)-5-((2-hydroxyethyl)amino)-3-oxo-2,3-dihydropyridazin-4-yl)acrylate 3007 (1.2 g, 95% purity, 32% yield) as yellow solid.

LCMS (ESI) calcd for C₁₉H₂₃N₃O₅ [M+H]+m/z 374.16, found 374.10.

### Preparation of ethyl 3-(2-((benzyloxy)methyl)-5-((2-hydroxyethyl)amino)-3-oxo-2,3-dihydropyridazin-4-yl)propanoate (3008)

To a solution of ethyl (E)-3-(2-((benzyloxy)methyl)-5-((2-hydroxyethyl)amino)-3-oxo-2,3-dihydropyridazin-4-yl)acrylate 3007 (1.2 g, 0.0032 mol) in DCM (5 mL) and MeOH (20 mL) at rt was added Pd/C (0.29 g, 0.0013 mol). The reaction mixture was stirred at rt for 4 h. The reaction solution was filtered and concentrated under reduced pressure to obtain ethyl 3-(2-((benzyloxy)methyl)-5-((2-hydroxyethyl)amino)-3-oxo-2,3-dihydropyridazin-4-yl)propanoate 3008 (1.1 g, 95% purity, 87% yield) as yellow oil.

LCMS (ESI) calcd for C₁₉H₂₅N₃O₅ [M+H]+m/z 376.18, found 376.05.

### Preparation of 6-((benzyloxy)methyl)-1-(2-hydroxyethyl)-4,6-dihydropyrido[2,3-d]pyridazine-2,5(1H,3H)-dione (3009)

To a solution of ethyl 3-(2-((benzyloxy)methyl)-5-((2-hydroxyethyl)amino)-3-oxo-2,3-dihydropyridazin-4-yl)propanoate 3008 (1.1 g, 0.0029 mol) in EtOH (50 mL) at rt were added 10% HCl aqueous solution (20 mL). The reaction mixture was stirred at 80° C. for 8 h. Then the reaction was quenched with water and adjusted pH to 7˜8 with 1 M NaOH aqueous at 0° C. The reaction solution was extracted with EtOAc (50 mL×3). The organic phase was concentrated under reduced pressure and the residue was purified by flash chromatography (eluting with PE/EtOAc=100:0 to 0:100) to give 6-((benzyloxy)methyl)-1-(2-hydroxyethyl)-4,6-dihydropyrido[2,3-d]pyridazine-2,5(1H,3H)-dione 3009 (0.85 g, 95% purity, 86% yield) as yellow solid.

¹H NMR (400 MHz, DMSO-d₆, ppm) δ: 8.20 (s, 1H), 7.40-7.23 (m, 5H), 5.44 (s, 2H), 4.89 (t, J=6.0 Hz, 1H), 4.64 (s, 1H), 3.94 (t, J=5.6 Hz, 1H), 3.58-3.50 (m, 1H), 2.73 (t, J=8.0 Hz, 1H), 2.60 (t, J=8.0 Hz, 1H).

LCMS (ESI) calcd for C₁₇H₁₉N₃O₄ [M+H]⁺ m/z 330.14, found 330.10.

### Preparation of (E)-6-((benzyloxy)methyl)-1-(2-((3-oxo-3-(4-(5-(trifluoromethyl)pyridin-2-yl)piperazin-1-yl)prop-1-en-1-yl)oxy)ethyl)-4,6-dihydropyrido[2,3-d]pyridazine-2,5(1H,3H)-dione (3010)

To a solution of 6-((benzyloxy)methyl)-1-(2-hydroxyethyl)-4,6-dihydropyrido[2,3-d]pyridazine-2,5(1H,3H)-dione 3009 (140 mg, 0.425 mmol) in DCM (100 mL) were added 1-(4-(5-(trifluoromethyl)pyridin-2-yl)piperazin-1-yl)prop-2-yn-1-one (120 mg, 0.425 mmol), P(n-Bu)₃ (135 mg, 0.425 mmol) successively at room temperature. The reaction mixture was stirred at room temperature for 2 h then concentrated under reduced pressure. The residue was purified by silica gel column chromatography (eluting with EtOAc/PE, 50% to 100%) to give (E)-6-((benzyloxy)methyl)-1-(2-((3-oxo-3-(4-(5-(trifluoromethyl)pyridin-2-yl)piperazin-1-yl)prop-1-en-1-yl)oxy)ethyl)-4,6-dihydropyrido[2,3-d]pyridazine-2,5(1H,3H)-dione 3010 (150 mg, 90% purity, 52% yield) as a yellow solid.

LCMS (ESI) calcd for C₃₀H₃₁F₃N₆O₅[M+H]⁺ m/z 613.23, found 613.10.

### Preparation of 6-((benzyloxy)methyl)-1-(2-(3-oxo-3-(4-(5-(trifluoromethyl)pyridin-2-yl)piperazin-1-yl)propoxy)ethyl)-4,6-dihydropyrido[2,3-d]pyridazine-2,5(1H,3H)-dione (3011)

To a solution of (E)-6-((benzyloxy)methyl)-1-(2-((3-oxo-3-(4-(5-(trifluoromethyl)pyridin-2-yl)piperazin-1-yl)prop-1-en-1-yl)oxy)ethyl)-4,6-dihydropyrido[2,3-d]pyridazine-2,5(1H,3H)-dione 3010 (140 mg, 0.229 mmol) in MeOH (20 mL) was added 10% Pd/C (140 mg). The mixture was evacuated and backfilled with hydrogen three times and then charged with hydrogen. The resulting mixture was stirred at rt for 2 h. Then the mixture was filtered through celite, concentrated under vacuum and purified by flash column chromatography (eluting with MeOH/DCM, 0% to 10%) to afford 6-((benzyloxy)methyl)-1-(2-(3-oxo-3-(4-(5-(trifluoromethyl)pyridin-2-yl)piperazin-1-yl)propoxy)ethyl)-4,6-dihydropyrido[2,3-d]pyridazine-2,5(1H,3H)-dione 3011 (115 mg, 90% purity, 74% yield) as a yellow solid.

LCMS (ESI) calcd for C₃₀H₃₃F₃N₆O₅[M+H]⁺ m/z 615.25, found 615.30.

### Preparation of 1-(2-(3-oxo-3-(4-(5-(trifluoromethyl)pyridin-2-yl)piperazin-1-yl)propoxy)ethyl)-4,6-dihydropyrido[2,3-d]pyridazine-2,5(1H,3H)-dione (compound 240)

A solution of 6-((benzyloxy)methyl)-1-(2-(3-oxo-3-(4-(5-(trifluoromethyl)pyridin-2-yl)piperazin-1-yl)propoxy)ethyl)-4,6-dihydropyrido[2,3-d]pyridazine-2,5(1H,3H)-dione 3011 (115 mg, 0.187 mmol) in TFA (5 mL) was stirred at room temperature for 2 h. The pH was adjusted to around 8 by progressively adding saturated aqueous NaHCO₃ at 0° C. The mixture was then extracted with EtOAc. The combined organic layers were concentrated under reduced pressure. The residue was purified on a Biotage Isolera One (C₁₈ column, eluting with 0% to 55% MeCN/H₂O containing 0.1% formic acid) to provide 1-(2-(3-oxo-3-(4-(5-(trifluoromethyl)pyridin-2-yl)piperazin-1-yl)propoxy)ethyl)-4,6-dihydropyrido[2,3-d]pyridazine-2,5(1H,3H)-dione 240 (34.0 mg, 93% purity, 35% yield) as a solid.

¹H NMR (400 MHz, DMSO-d₆, ppm) δ:12.91 (s, 1H), 8.43 (s, 1H), 8.07 (s, 1H), 7.83 (dd, J=9.2, 2.4 Hz, 1H), 6.96 (d, J=9.2 Hz, 1H), 4.02 (t, J=5.6 Hz, 2H), 3.66-3.61 (m, 6H), 3.56-3.50 (m, 6H), 2.71-2.63 (m, 2H), 2.62-2.55 (m, 2H), 2.55-2.51 (m, 2H).

LCMS (ESI) calcd for C₂₂H₂₅F₃N₆O₄[M+H]⁺ m/z 495.19, found 495.25.

### 22. Synthesis of 1-(1-(3-(4-(5-fluoropyrimidin-2-yl)piperazin-1-yl)-3-oxopropoxy)propan-2-yl)-3-(trifluoromethyl)-1,5-dihydro-4H-pyrazolo[3,4-d]pyridazin-4-one (compounds 248 and 249)

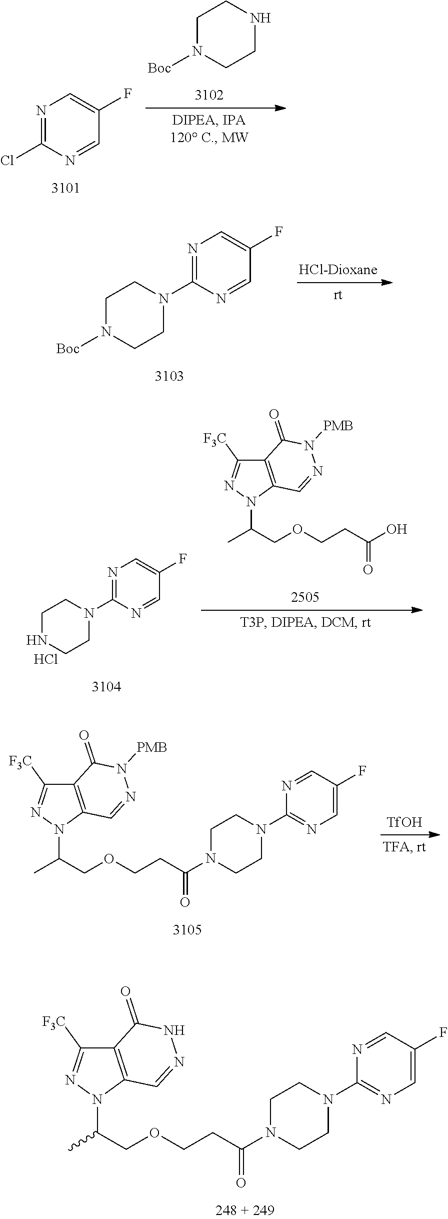

### Preparation of tert-butyl 4-(5-fluoropyrimidin-2-yl)piperazine-1-carboxylate (3103)

2-chloro-5-fluoropyrimidine 3101 (500 mg, 3.77 mmol), tert-butyl piperazine-1-carboxylate 3102 (843.28 mg, 4.53 mmol) and DIPEA (975.28 mg, 7.55 mmol) were dissolved in IPA (10 mL). The resulting mixture was heated at 120° C. for 2 hours in a microwave reactor. Then the mixture was cooled to room temperature, diluted with water (30 mL), and extracted with EtOAc (2×30 mL). The organic phases were dried over Na₂SO₄, filtered, and concentrated under reduced pressure. The residue was purified by silica gel column chromatography (eluting with EtOAc/PE, 0 to 20%) to afford tert-butyl 4-(5-fluoropyrimidin-2-yl)piperazine-1-carboxylate 3103 (300 mg, 85% purity, 24% yield) as a white solid.

LCMS (ESI) calcd for C₁₃H₁₉FN₄O₂[M−t-Bu+H]⁺ m/z 227.16, found 227.0.

### Preparation of 5-fluoro-2-(piperazin-1-yl)pyrimidine hydrochloride (3104)

To a 50 mL round-bottom flask was added tert-butyl 4-(5-fluoropyrimidin-2-yl)piperazine-1-carboxylate 3103 (300 mg, 1.059 mmol) and HCl-dioxane (4 M, 15 mL). The mixture was stirred for 5 hours at room temperature. The reaction mixture was concentrated to afford 5-fluoro-2-(piperazin-1-yl)pyrimidine hydrochloride 3104 (225 mg, 85% purity, 82.6% yield) as white solid.

LCMS (ESI) calcd for C₈H₁₁FN₄ [M+H]⁺ m/z 183.11, found 183.0.

### Preparation of 1-(1-(3-(4-(5-fluoropyrimidin-2-yl)piperazin-1-yl)-3-oxopropoxy)propan-2-yl)-5-(4-methoxybenzyl)-3-(trifluoromethyl)-1,5-dihydro-4H-pyrazolo[3,4-d]pyridazin-4-one (3105)

To a solution of 5-fluoro-2-(piperazin-1-yl) pyrimidine hydrochloride 3104 (200 mg, 0.44 mmol) in DCM (10 mL) was added 3-(2-(5-(4-methoxybenzyl)-4-oxo-3-(trifluoromethyl)-4,5-dihydro-1H-pyrazolo[3,4-d]pyridazin-1-yl)propoxy)propanoic acid 2505 (116 mg, 0.52 mmol), T3P (50% in EtOAc, 560 mg, 0.88 mmol) and DIPEA (284 mg, 2.2 mmol). The reaction mixture was stirred at room temperature for 2 hours. After the reaction was completed, the resulting mixture was diluted with water and extracted with DCM (30 mL×3). The combined organic layers were dried over Na₂SO₄ and concentrated under reduced pressure. The residue was purified by prep-HPLC (columns: Gemini 5 um C₁₈ 150×21.2 mm, mobile phase: ACN-H₂O (0.1% FA), gradient: 50%-95%) to give 1-(1-(3-(4-(5-fluoropyrimidin-2-yl)piperazin-1-yl)-3-oxopropoxy)propan-2-yl)-5-(4-methoxybenzyl)-3-(trifluoromethyl)-1,5-dihydro-4H-pyrazolo[3,4-d]pyridazin-4-one 3105 (116 mg, 98% purity, 42% yield) as a white solid.

LCMS (ESI) calcd for C₂₈H₃₀F₄N₈O₄ [M+H]⁺ m/z 619.24, found 619.0.

### Preparation of 1-(1-(3-(4-(5-fluoropyrimidin-2-yl)piperazin-1-yl)-3-oxopropoxy)propan-2-yl)-3-(trifluoromethyl)-1,5-dihydro-4H-pyrazolo[3,4-d]pyridazin-4-one (compounds 248 and 249)

TfOH (0.2 mL) was added to a solution of 1-(1-(3-(4-(5-fluoropyrimidin-2-yl)piperazin-1-yl)-3-oxopropoxy)propan-2-yl)-5-(4-methoxybenzyl)-3-(trifluoromethyl)-1,5-dihydro-4H-pyrazolo[3,4-d]pyridazin-4-one 3105 (100 mg, 0.16 mmol) in TFA (10 mL). The reaction mixture was stirred at room temperature for 10 h then adjusted to pH=8 by progressively adding saturated aqueous NaHCO₃ at 0° C. Then the mixture was extracted with DCM (30 mL×3). The combined organic layers were washed with brine, dried over Na₂SO₄, and concentrated under reduced pressure. The residue was purified twice by C₁₈ column (Agela 40 g, mobile phase: ACN-H₂O (0.1% FA), gradient: 40%-50%) to give 1-(1-(3-(4-(5-fluoropyrimidin-2-yl)piperazin-1-yl)-3-oxopropoxy)propan-2-yl)-3-(trifluoromethyl)-1,5-dihydro-4H-pyrazolo[3,4-d]pyridazin-4-one (compounds 248 and 249) (50 mg, 98% purity, 61% yield) as a white solid.

LCMS (ESI) calcd for C₂₀H₂₂F₄N₈O₃[M+H]⁺ m/z 499.19, found 499.0.

### Chiral resolution of 1-(1-(3-(4-(5-fluoropyrimidin-2-yl)piperazin-1-yl)-3-oxopropoxy)propan-2-yl)-3-(trifluoromethyl)-1,5-dihydro-4H-pyrazolo[3,4-d]pyridazin-4-one (compounds 248 and 249)

Compounds 248 and 249 were separated by SFC (Column: DAICEL OJ-H 4.6 mm I.D.×250 mmL 5 m; Mobile phase: CO₂/MeOH (0.1% FA)=60/40) and concentrated under reduced pressure to afford the first fraction as 248 (16.7 mg, 98% purity, 100% ee, white solid) and the second fraction as 249 (19.3 mg, 98% purity, 97% ee, white solid).

### Compound 248

¹H NMR (400 MHz, DMSO-d₆, ppm) δ: 12.84 (s, 1H), 8.65 (s, 1H), 8.47 (s, 2H), 5.26-5.08 (m, 1H), 3.78-3.72 (m, 1H), 3.71-3.62 (m, 2H), 3.62-3.55 (m, 4H), 3.54-3.46 (m, 1H), 3.46-3.36 (m, 4H), 2.45-2.38 (m, 2H), 1.49 (d, J=6.8 Hz, 3H).

LCMS (ESI) calcd for C₂₀H₂₂F₄N₈O₃[M+H]⁺ m/z 499.19, found 499.0.

### Compound 249

¹H NMR (400 MHz, DMSO-d₆, ppm) δ: 12.84 (s, 1H), 8.65 (s, 1H), 8.47 (s, 2H), 5.22-5.08 (m, 1H), 3.79-3.71 (m, 1H), 3.71-3.63 (m, 2H), 3.63-3.55 (m, 4H), 3.54-3.48 (m, 1H), 3.46-3.37 (m, 4H), 2.46-2.39 (m, 2H), 1.49 (d, J=6.8 Hz, 3H).

LCMS (ESI) calcd for C₂₀H₂₂F₄N₈O₃[M+H]⁺ m/z 499.19, found 499.0.

### 23. Synthesis of 6-(2-(3-oxo-3-(4-(5-(trifluoromethyl)pyrimidin-2-yl)piperazin-1-yl)propoxy)ethyl)-8-(trifluoromethyl)-3,4-dihydropyrrolo[1,2-a]pyrazin-1(2H)-one (compound 316)

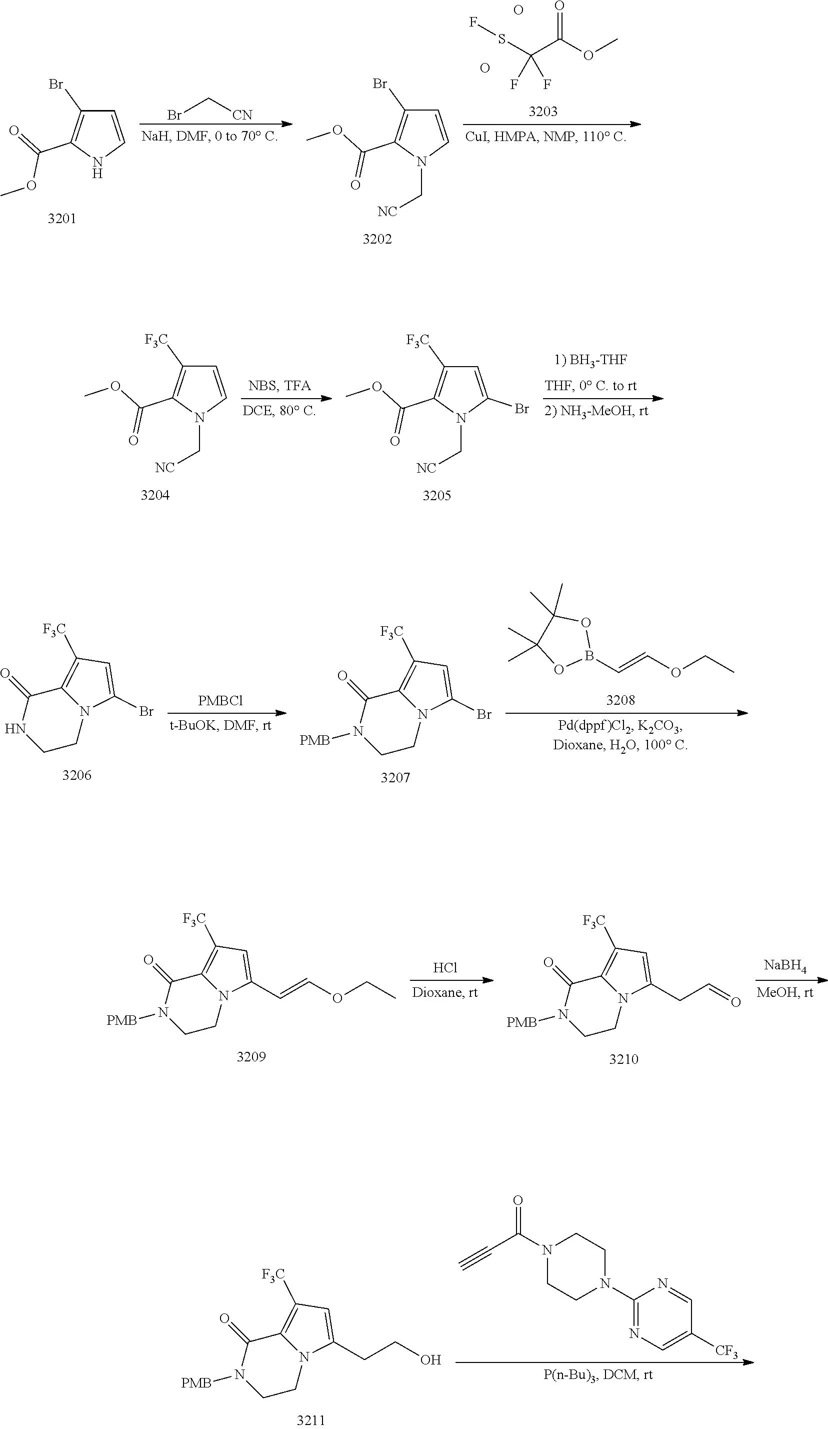

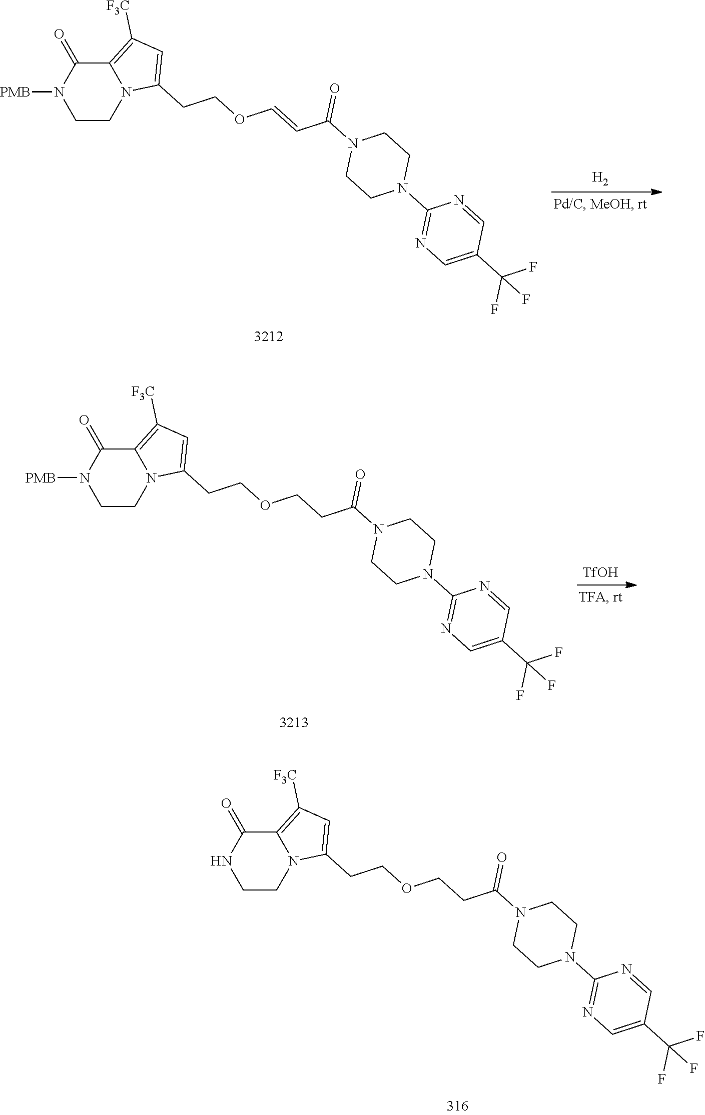

### Preparation of methyl 3-bromo-1-(cyanomethyl)-1H-pyrrole-2-carboxylate (3202)

To a suspension of NaH (60%, 0.98 g, 0.0245 mol) in DMF (50 mL) was added dropwise a solution of methyl 3-bromo-1H-pyrrole-2-carboxylate 3201 (1 g, 0.0049 mol) and 2-bromoacetonitrile (2.94 g, 0.0245 mmol) in DMF (10 mL) at 0° C. under N₂ atmosphere. The reaction mixture was warmed to 70° C. and kept stirring at 70° C. for an additional 2 h. After completion of reaction, the reaction mixture was cooled to rt and then poured into cold saturated aqueous NH₄Cl. The resulting solution was extracted with EtOAc (50 mL×3). The combined organic layer was washed with brine, dried over Na₂SO₄ and concentrated under reduced pressure. The residue was purified by flash silica chromatography (eluting with EtOAc/PE, 0% to 10%) to give methyl 3-bromo-1-(cyanomethyl)-1H-pyrrole-2-carboxylate 3202 (0.8 g, 90% purity, 31% yield) as a white solid.

### Preparation of methyl 1-(cyanomethyl)-3-(trifluoromethyl)-1H-pyrrole-2-carboxylate (3204)

To a stirred solution of methyl 3-bromo-1-(cyanomethyl)pyrrole-2-carboxylate 3202 (800 mg, 3.29 mmol), CuI (626 mg, 3.29 mmol), HMPA (2949 mg, 16.46 mmol) in NMP (20 mL) was added methyl 2,2-difluoro-2-(fluorosulfonyl)acetate 3203 (3161 mg, 16.46 mmol) at rt. The solution was then stirred at 110° C. for 3 h. The resulting reaction solution was filtered, and the filtrate was purified directly by flash silica chromatography (eluting with EtOAc/PE, 10% to 20%) to give 1-(cyanomethyl)-3-(trifluoromethyl)-1H-pyrrole-2-carboxylate 3204 (250 mg, 90% purity, 29% yield) as yellow solid.

### Preparation of methyl 5-bromo-1-(cyanomethyl)-3-(trifluoromethyl)-1H-pyrrole-2-carboxylate (3205)

To a solution of methyl 1-(cyanomethyl)-3-(trifluoromethyl)-1H-pyrrole-2-carboxylate 3204 (100 mg, 0.429 mmol) in DCE (5 mL) was added NBS (114 mg, 0.643 mmol) and TFA (24 mg, 0.214 mmol) at rt. The mixture was heated at 80° C. for 16 hours. The resulting mixture was diluted with water (30 mL) and extracted with DCM (10 mL×3). The combine organic phases were dried over sodium sulfate, concentrated, and purified by silica gel column chromatography (eluting with EtOAc/PE, 10% to 20%) to give methyl 5-bromo-1-(cyanomethyl)-3-(trifluoromethyl)-1H-pyrrole-2-carboxylate 3205 (40 mg, 90% purity, 26% yield) as a white solid.

### Preparation of 6-bromo-8-(trifluoromethyl)-3,4-dihydropyrrolo[1,2-a]pyrazin-1(2H)-one (3206)

To a solution of methyl 5-bromo-1-(cyanomethyl)-3-(trifluoromethyl)-1H-pyrrole-2-carboxylate 3205 (600 mg, 1.9289 mmol) in THF (10 mL) was added slowly BH₃-THF (1 M, 9.6 mL, 9.6 mmol) at 0° C. After completion of addition, the reaction solution was warmed to rt and kept stirring at rt for an additional 16 h. The resulting reaction solution was added dropwise MeOH (50 mL) to quench the BH₃-THF at rt and then concentrated under reduced pressure. The residue was diluted with NH₃-MeOH (7 M, 12.3 mL) and kept stirring at rt overnight. LCMS monitored the formation of product, the reaction mixture was concentrated, and purified by silica gel column chromatography (eluting with EtOAc/PE, 30% to 70%) to give 6-bromo-8-(trifluoromethyl)-3,4-dihydropyrrolo[1,2-a]pyrazin-1(2H)-one 3206 (250 mg, purity 90%, 41% yield) as a white solid.

LCMS (ESI) calcd for C₈H₆BrF₃N₂O [M+H]⁺ m/z 282.96, found 282.95.

### Preparation of 6-bromo-2-(4-methoxybenzyl)-8-(trifluoromethyl)-3,4-dihydropyrrolo[1,2-a]pyrazin-1(2H)-one (3207)

To a solution of t-BuOK (0.2 g, 1.77 mmol) in DMF (10 mL) was added slowly a solution of 6-bromo-8-(trifluoromethyl)-3,4-dihydropyrrolo[1,2-a]pyrazin-1(2H)-one 3206 (0.25 g, 0.883 mmol) and PMBCl (0.28 g, 1.77 mmol) in DMF (5 mL) at 0° C. under N₂ atmosphere. After completion of addition, the reaction was warmed to rt and kept stirring for an additional 1.5 h. The resulting reaction mixture was poured into cold saturated aqueous NH₄Cl and stirred for 5 min. Then the solution was extracted with EtOAc (50 mL×3). The combined organic layer was washed with bine (30 mL×3), dried over Na₂SO₄ and concentrated under reduced pressure. The residue was purified by flash silica chromatography (eluting with EtOAc/PE, 20% to 40%) to give 6-bromo-2-(4-methoxybenzyl)-8-(trifluoromethyl)-3,4-dihydropyrrolo[1,2-a]pyrazin-1(2H)-one 3207 (0.2 g, 90% purity, 50% yield) as a white solid.

LCMS (ESI) calcd for C₁₆H₁₄BrF₃N₂O₂ [M+H]⁺ m/z 403.02, found 403.10.

### Preparation of (E)-6-(2-ethoxyvinyl)-2-(4-methoxybenzyl)-8-(trifluoromethyl)-3,4-dihydropyrrolo[1,2-a]pyrazin-1(2H)-one (3209)

A suspension of 6-bromo-2-(4-methoxybenzyl)-8-(trifluoromethyl)-3,4-dihydropyrrolo[1,2-a]pyrazin-1(2H)-one 3207 (200 mg, 0.50 mmol), K₂CO₃ (137 mg, 0.99 mmol) and Pd(dppf)Cl₂ (36 mg, 0.496 mmol) in Dioxane/H₂O (10/1, 10 mL) was heated at 100° C. for 2 h under N₂. After cooling to rt, the reaction mixture was poured into ice water and then extracted with EtOAc (30 mL×3). The combined organic layers were washed with water, dried over Na₂SO₄, and concentrated under reduced pressure. The residue was purified by flash silica chromatography (eluting with EtOAc/PE, 0 to 35%) to give (E)-6-(2-ethoxyvinyl)-2-(4-methoxybenzyl)-8-(trifluoromethyl)-3,4-dihydropyrrolo[1,2-a]pyrazin-1(2H)-one 3209 (120 mg, 90% purity, 55% yield) as a white solid.

LCMS (ESI) calcd for C₂₀H₂₁F₃N₂O₃ [M+H]⁺ m/z 395.15, found 395.10.

### Preparation of 2-(2-(4-methoxybenzyl)-1-oxo-8-(trifluoromethyl)-1,2,3,4-tetrahydropyrrolo [1,2-a]pyrazin-6-yl)acetaldehyde (3210)

To a solution of (E)-6-(2-ethoxyvinyl)-2-(4-methoxybenzyl)-8-(trifluoromethyl)-3,4-dihydropyrrolo[1,2-a]pyrazin-1(2H)-one 3209 (120 mg, 0.304 mmol) in DCM (8 mL) was added HCl in dioxane (4 M, 2 mL) dropwise at rt. The mixture was stirred at rt for 5 min. The resulting mixture was concentrated under reduced pressure to give 2-(2-(4-methoxybenzyl)-1-oxo-8-(trifluoromethyl)-1,2,3,4-tetrahydropyrrolo [1,2-a]pyrazin-6-yl)acetaldehyde 3210 (100 mg, 50% purity, 44% yield) as a yellow oil.

LCMS (ESI) calcd for C₁₈H₁₇F₃N₂O₃[M+H]⁺ m/z 367.12, found 367.10.

### Preparation of 6-(2-hydroxyethyl)-2-(4-methoxybenzyl)-8-(trifluoromethyl)-3,4-dihydropyrrolo[1,2-a]pyrazin-1(2H)-one (3211)

To a solution of 2-(2-(4-methoxybenzyl)-1-oxo-8-(trifluoromethyl)-1,2,3,4-tetrahydropyrrolo [1,2-a]pyrazin-6-yl)acetaldehyde 3210 (100 mg, 0.273 mmol) in MeOH (10 mL) was added NaBH₄ (20 mg, 0.546 mmol) at rt. The reaction mixture was stirred at rt for 10 min. The resulting reaction solution was quenched with water and then extracted with EtOAc (50 mL×3). The combined organic layers were dried over Na₂SO₄, concentrated under reduced pressure. The residue was purified by flash chromatography (eluting with EtOAc/PE, 30% to 60%) to obtained 6-(2-hydroxyethyl)-2-(4-methoxybenzyl)-8-(trifluoromethyl)-3,4-dihydropyrrolo[1,2-a]pyrazin-1(2H)-one 3211 (40 mg, 90% purity, 35% yield) as a white solid.

LCMS (ESI) calcd for C₁₈H₁₉F₃N₂O₃[M+H]⁺ m/z 369.14, found 369.00.

### Preparation of (E)-2-(4-methoxybenzyl)-6-(2-((3-oxo-3-(4-(5-(trifluoromethyl)pyrimidin-2-yl)piperazin-1-yl)prop-1-en-1-yl)oxy)ethyl)-8-(trifluoromethyl)-3,4-dihydropyrrolo[1,2-a]pyrazin-1(2H)-one (3212)

To a solution of 6-(2-hydroxyethyl)-2-(4-methoxybenzyl)-8-(trifluoromethyl)-3,4-dihydropyrrolo[1,2-a]pyrazin-1(2H)-one 3211 (40 mg, 0.11 mmol) in DCM (5 mL) were added 1-(4-(5-(trifluoromethyl)pyrimidin-2-yl)piperazin-1-yl)prop-2-yn-1-one (34 mg, 0.12 mmol) and P(n-Bu)₃ (2 mg, 0.011 mmol) at rt successively. The reaction mixture was stirred at room temperature for 2 h. The reaction mixture was concentrated under reduced pressure. The residue was purified by flash column chromatography (eluting with MeOH/DCM, 0% to 5%) to afford (E)-2-(4-methoxybenzyl)-6-(2-((3-oxo-3-(4-(5-(trifluoromethyl)pyrimidin-2-yl)piperazin-1-yl)prop-1-en-1-yl)oxy)ethyl)-8-(trifluoromethyl)-3,4-dihydropyrrolo[1,2-a]pyrazin-1(2H)-one 3212 (60 mg, 90% purity, 76% yield) as a white solid.

LCMS (ESI) calcd for C₃₀H₃₀F₆N₆O₄[M+Na]⁺ m/z 675.22, found 675.25.

### Preparation of 2-(4-methoxybenzyl)-6-(2-(3-oxo-3-(4-(5-(trifluoromethyl)pyrimidin-2-yl)piperazin-1-yl)propoxy)ethyl)-8-(trifluoromethyl)-3,4-dihydropyrrolo[1,2-a]pyrazin-1(2H)-one (3213)

A solution of (E)-2-(4-methoxybenzyl)-6-(2-((3-oxo-3-(4-(5-(trifluoromethyl)pyrimidin-2-yl)piperazin-1-yl)prop-1-en-1-yl)oxy)ethyl)-8-(trifluoromethyl)-3,4-dihydropyrrolo[1,2-a]pyrazin-1(2H)-one 3212 (60 mg, 0.09 mmol) and Pd/C (10%, 6 mg) in MeOH (6 mL) was stirred at rt for 16 h under H₂ atmosphere. The resulting solution was filtered, and the filter cake was washed with MeOH three times. The filtrate was concentrated under reduced pressure to afford 2-(4-methoxybenzyl)-6-(2-(3-oxo-3-(4-(5-(trifluoromethyl)pyrimidin-2-yl)piperazin-1-yl)propoxy)ethyl)-8-(trifluoromethyl)-3,4-dihydropyrrolo[1,2-a]pyrazin-1(2H)-one 3213 (50 mg, 90% purity, 74% yield) as a white solid.

LCMS (ESI) calcd for C₃₀H₃₂F₆N₆O₄[M+H]⁺ m/z 655.24, found 655.35.

### Preparation of 6-(2-(3-oxo-3-(4-(5-(trifluoromethyl)pyrimidin-2-yl)piperazin-1-yl)propoxy)ethyl)-8-(trifluoromethyl)-3,4-dihydropyrrolo[1,2-a]pyrazin-1(2H)-one (compound 316)

TfOH (0.5 mL) was added to a solution of 2-(4-methoxybenzyl)-6-(2-(3-oxo-3-(4-(5-(trifluoromethyl)pyrimidin-2-yl)piperazin-1-yl)propoxy)ethyl)-8-(trifluoromethyl)-3,4-dihydropyrrolo[1,2-a]pyrazin-1(2H)-one 3213 (50 mg, 0.076 mmol) in TFA (2 mL). The reaction mixture was stirred at rt for 30 min. The pH of the resulting mixture was adjusted to around 8 by progressively adding saturated NaHCO₃ solution at 0° C., and then extracted with DCM (50 mL×3). The combined organic layers were washed with brine, dried over Na₂SO₄ and concentrated under reduced pressure. The residue was purified with C₁₈ column (Agela 40 g, mobile phase: ACN-H₂O (0.1% FA), gradient: 40-60) to give 6-(2-(3-oxo-3-(4-(5-(trifluoromethyl)pyrimidin-2-yl)piperazin-1-yl)propoxy)ethyl)-8-(trifluoromethyl)-3,4-dihydropyrrolo[1,2-a]pyrazin-1(2H)-one 316 (14.5 mg, 94% purity, 34% yield) as a white solid.

¹H NMR (400 MHz, DMSO-d₆, ppm) δ: 8.73 (s, 2H), 7.92 (s, 1H), 6.32 (s, 1H), 4.08-4.01 (m, 2H), 3.86-3.75 (m, 4H), 3.69-3.60 (m, 4H), 3.58-3.52 (m, 4H), 3.50-3.44 (m, 2H), 2.83 (t, J=6.4 Hz, 2H), 2.61 (t, J=6.4 Hz, 2H).

LCMS (ESI) calcd for C₂₂H₂₄F₆N₆O₃[M+H]⁺ m/z 535.18, found 535.05.

### 24. Synthesis of N-methyl-2-(2-(4-oxo-3-(trifluoromethyl)-4,5-dihydro-1H-pyrazolo[3,4-d]pyridazin-1-yl)ethoxy)-N-(1-(5-(trifluoromethyl)pyrimidin-2-yl)piperidin-4-yl)acetamide (compound 317)

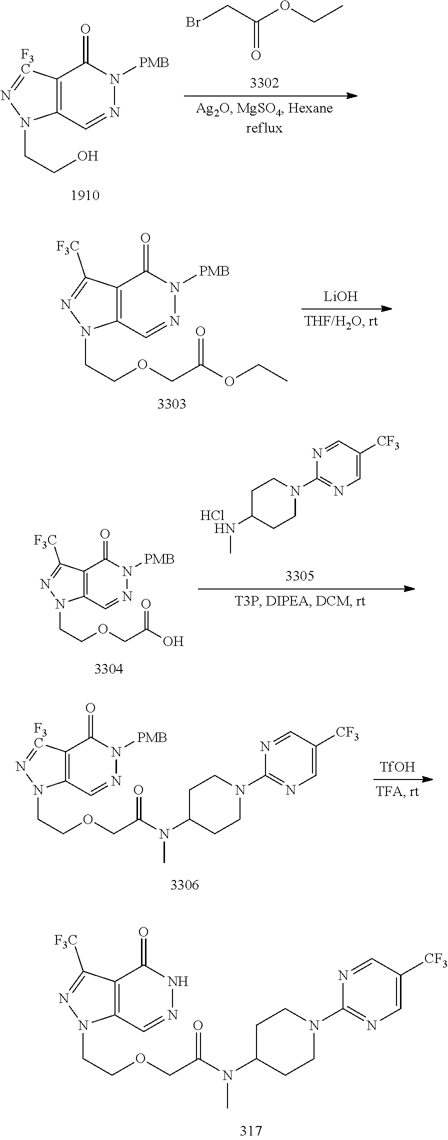

### Preparation of ethyl 2-(2-(5-(4-methoxybenzyl)-4-oxo-3-(trifluoromethyl)-4,5-dihydro-1H-pyrazolo[3,4-d]pyridazin-1-yl)ethoxy)acetate (3303)

To a solution of 1-(2-hydroxyethyl)-5-(4-methoxybenzyl)-3-(trifluoromethyl)-1,5-dihydro-4H-pyrazolo[3,4-d]pyridazin-4-one 1910 (100 mg, 0.27 mmol) in n-Hexane (2 mL) were added Ag₂O (252 mg, 1.09 mmol) and MgSO₄ (130 mg, 1.08 mmol) successively at 80° C. under N₂. After the reaction mixture was refluxed for 1 h, ethyl 2-bromoacetate 3302 (317 mg, 1.90 mmol) was added to the solution. The mixture was refluxed for additional 18 h. Then it was cooled to rt. The reaction mixture was poured into cold water and then extracted with EtOAc (50 mL×3), dried over anhydrous sodium sulfate and concentrated in vacuo. The residue was purified by Flash chromatography (eluting with PE/EtOAc=70:30 to 30:70) to afford ethyl 2-(2-(5-(4-methoxybenzyl)-4-oxo-3-(trifluoromethyl)-4,5-dihydro-1H-pyrazolo[3,4-d]pyridazin-1-yl)ethoxy)acetate 3303 (80 mg, 90% purity, 58% yield) as colorless oil.

LCMS (ESI) calcd for C₂₀H₂₁F₃N₄O₅[M+H]⁺ m/z 455.15, found 455.20.

### Preparation of 2-(2-(5-(4-methoxybenzyl)-4-oxo-3-(trifluoromethyl)-4,5-dihydro-1H-pyrazolo[3,4-d]pyridazin-1-yl)ethoxy)acetic acid (3304)

LiOH (13 mg, 0.53 mmol) was added to a solution of ethyl 2-(2-(5-(4-methoxybenzyl)-4-oxo-3-(trifluoromethyl)-4,5-dihydro-1H-pyrazolo[3,4-d]pyridazin-1-yl)ethoxy)acetate 3303 (80 mg, 0.18 mmol) in THF/H₂O (THF:H₂O=3:1, 4 mL). After stirring at rt for 3 h, the reaction mixture was concentrated under reduced pressure to remove THF. The obtained aqueous was acidified with HCl solution (1 mol/L) and extracted with DCM (20 mL×3). The combined organic layers were washed with brine, dried over Na₂SO₄ and concentrated under reduced pressure to give 2-(2-(5-(4-methoxybenzyl)-4-oxo-3-(trifluoromethyl)-4,5-dihydro-1H-pyrazolo[3,4-d]pyridazin-1-yl)ethoxy)acetic acid 3304 (70 mg, 80% purity, 74% yield) as a colorless oil.

LCMS (ESI) calcd for C₁₈H₁₇F₃N₄O₅[M+H]⁺ m/z 427.12, found 427.25.

### Preparation of 2-(2-(5-(4-methoxybenzyl)-4-oxo-3-(trifluoromethyl)-4,5-dihydro-1H-pyrazolo[3,4-d]pyridazin-1-yl)ethoxy)-N-methyl-N-(1-(5-(trifluoromethyl)pyrimidin-2-yl)piperidin-4-yl)acetamide (3306)

To a solution of 2-(2-(5-(4-methoxybenzyl)-4-oxo-3-(trifluoromethyl)-4,5-dihydro-1H-pyrazolo[3,4-d]pyridazin-1-yl)ethoxy)acetic acid 3304 (70 mg, 0.16 mmol) in DCM (2 mL) were added N-methyl-1-(5-(trifluoromethyl)pyrimidin-2-yl)piperidin-4-amine hydrochloride 3305 (51 mg, 0.20 mmol), DIPEA (106 mg, 0.82 mmol), T3P (50% in EtOAc, 157 mg, 0.25 mmol) at room temperature successively. The mixture was stirred at room temperature for 2 h. The resulting mixture was diluted with water and extracted with DCM (30 mL×3). The combined organic layers were dried over Na₂SO₄ and concentrated under reduced pressure. The residue was purified by C₁₈ column (mobile phase: ACN-H₂O (0.1% FA), gradient: 0%-100%) to obtain 2-(2-(5-(4-methoxybenzyl)-4-oxo-3-(trifluoromethyl)-4,5-dihydro-1H-pyrazolo[3,4-d]pyridazin-1-yl)ethoxy)-N-methyl-N-(1-(5-(trifluoromethyl)pyrimidin-2-yl)piperidin-4-yl)acetamide 3306 (80 mg, 90% purity, 65% yield) as white solid.

LCMS (ESI) calcd for C₂₉H₃₀F₆N₈O₄ [M+H]+m/z 669.23, found 669.33.

### Preparation of N-methyl-2-(2-(4-oxo-3-(trifluoromethyl)-4,5-dihydro-1H-pyrazolo[3,4-d]pyridazin-1-yl)ethoxy)-N-(1-(5-(trifluoromethyl)pyrimidin-2-yl)piperidin-4-yl)acetamide (compound 317)

To a solution of 2-(2-(5-(4-methoxybenzyl)-4-oxo-3-(trifluoromethyl)-4,5-dihydro-1H-pyrazolo[3,4-d]pyridazin-1-yl)ethoxy)-N-methyl-N-(1-(5-(trifluoromethyl)pyrimidin-2-yl)piperidin-4-yl)acetamide 3306 (80 mg, 0.12 mmol) in TFA (3 mL) was added TfOH (1 mL) dropwise at rt. After completion of addition, the reaction solution was stirred at rt for 20 min. The resulting mixture was adjusted pH to 8 with saturated aqueous NaHCO₃ at 0° C., then extracted with DCM (30 mL×3). The combined organic phases were concentrated under reduced pressure. The residue was purified by prep-HPLC (Gemini 5 um C₁₈ column, 150×21.2 mm, eluting with 30% to 95% MeCN/H₂O containing 0.1% FA) and C₁₈ column (mobile phase: ACN-H₂O (0.1% FA), gradient: 0%-100%) to obtain N-methyl-2-(2-(4-oxo-3-(trifluoromethyl)-4,5-dihydro-1H-pyrazolo[3,4-d]pyridazin-1-yl)ethoxy)-N-(1-(5-(trifluoromethyl)pyrimidin-2-yl)piperidin-4-yl)acetamide 317 (9.9 mg, 100% purity, 15% yield) as a white solid.

¹H NMR (400 MHz, DMSO-d₆, ppm) δ:12.62 (s, 1H), 8.62 (s, 2H), 8.59 (s, 1H), 4.80 (d, J=13.2 Hz, 2H), 4.73 (t, J=5.2 Hz, 2H), 4.59-4.05 (m, 3H), 3.91 (t, J=4.8 Hz, 2H), 2.96 (t, J=12.0 Hz, 2H), 2.59 (s, 3H), 1.66-1.53 (m, 4H).

LCMS (ESI) calcd for C₂₁H₂₂F₆N₈O₃[M+H]⁺ m/z 549.17, found 549.25.

### 25. Synthesis of 2-(4-(3-(2-(4-oxo-3-(trifluoromethyl)-4,5-dihydro-1H-pyrazolo[3,4-d]pyridazin-1-yl) propoxy) propanoyl) piperazin-1-yl) pyrimidine-5-carbonitrile (compounds 321 and 322)

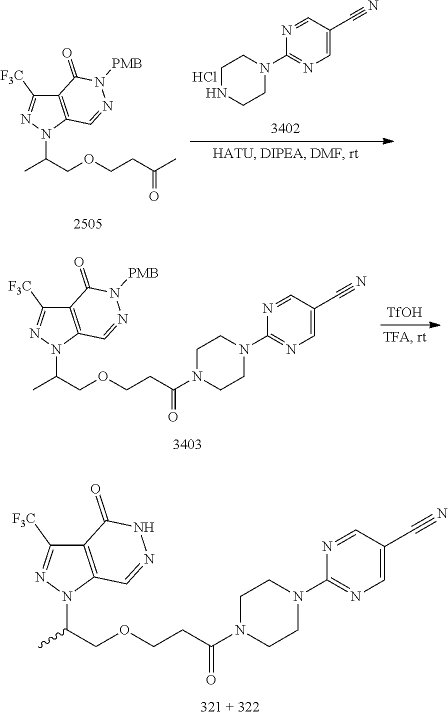

### Preparation of 2-(4-(3-(2-(5-(4-methoxybenzyl)-4-oxo-3-(trifluoromethyl)-4,5-dihydro-1H-pyrazolo[3,4-d]pyridazin-1-yl) propoxy) propanoyl) piperazin-1-yl) pyrimidine-5-carbonitrile (3403)

To a solution of 3-(2-(5-(4-methoxybenzyl)-4-oxo-3-(trifluoromethyl)-4,5-dihydro-1H-pyrazolo[3,4-d]pyridazin-1-yl) propoxy) propanoic acid 2505 (150 mg, 0.33 mmol) in DMF (5 mL) were added 2-(piperazin-1-yl) pyrimidine-5-carbonitrile hydrochloride 3402 (112 mg, 0.50 mmol), DIPEA (2123 mg, 1.65 mmol), HATU (158 mg, 0.50 mmol) at rt successively. The reaction mixture was stirred at rt for 1 h. The reaction mixture was diluted with water (15 mL) and extracted with EtOAc (15 mL×3). The combined organic layer was washed with brine, dried over Na₂SO₄. The combined organic phases were concentrated and purified by C₁₈ column (Agela 40 g, mobile phase: ACN-H₂O (0.1% FA), gradient: 47-50) to afford 2-(4-(3-(2-(5-(4-methoxybenzyl)-4-oxo-3-(trifluoromethyl)-4,5-dihydro-1H-pyrazolo[3,4-d]pyridazin-1-yl) propoxy) propanoyl) piperazin-1-yl) pyrimidine-5-carbonitrile 3403 (90 mg, 95% purity, 41% yield) as a yellow solid.

LCMS (ESI) calcd for C₂₉H₃₀F₃N₉O₄[M+H]⁺ m/z 626.24, found 626.39

### Preparation of 2-(4-(3-(2-(4-oxo-3-(trifluoromethyl)-4,5-dihydro-1H-pyrazolo[3,4-d]pyridazin-1-yl) propoxy) propanoyl) piperazin-1-yl) pyrimidine-5-carbonitrile (compounds 321 and 322)

To a stirred solution of 2-(4-(3-(2-(5-(4-methoxybenzyl)-4-oxo-3-(trifluoromethyl)-4,5-dihydro-1H-pyrazolo[3,4-d]pyridazin-1-yl) propoxy) propanoyl) piperazin-1-yl) pyrimidine-5-carbo nitrile 3403 (80 mg, 0.13 mmol) in TFA (5 mL) was added TfOH (0.1 mL) at rt. The mixture was stirred at rt for 10 min. The resulting solution was adjusted to pH 7-8 with saturated aqueous NaHCO₃ at 0° C. and extracted with DCM. The combined organic phases were washed with water and brine, dried over sodium sulfate, concentrated under vacuum, and purified by C₁₈ column (Agela 40 g, mobile phase: ACN-H₂O (0.1% FA), gradient: 45-47) to afford 2-(4-(3-(2-(4-oxo-3-(trifluoromethyl)-4,5-dihydro-1H-pyrazolo[3,4-d]pyridazin-1-yl) propoxy) propanoyl) piperazin-1-yl) pyrimidine-5-carbonitrile (a mixture of compounds 321 and 322) (20 mg, 95% purity) as a green solid.

### Chiral resolution of 2-(4-(3-(2-(4-oxo-3-(trifluoromethyl)-4,5-dihydro-1H-pyrazolo[3,4-d]pyridazin-1-yl) propoxy) propanoyl) piperazin-1-yl) pyrimidine-5-carbonitrile (compounds 321 and 322)

Compounds 321 and 322 were separated by SFC (Column: Daicel OJ-H 20 mm I.D.×250 mmL 5 m; Mobile phase: CO₂/MeOH (0.1% FA)=60/40) and concentrated under reduced pressure to afford the first fraction as 321 (6.3 mg, 98% purity, 100% ee, green solid) and the second fraction as 322 (8.9 mg, 98% purity, 100% ee, green solid).

### Compound 321

¹H NMR (400 MHz, DMSO-d₆, ppm) δ:12.83 (s, 1H), 8.78 (s, 2H), 8.64 (s, 1H), 5.25-5.06 (m, 1H), 3.77-3.65 (m, 7H), 3.53-3.40 (m, 5H), 2.47-2.41 (m, 2H), 1.49 (d, J=6.8 Hz, 3H).

LCMS (ESI) calcd. for C₂₁H₂₂F₃N₉O₃[M+H]⁺ m/z 506.18, found 506.25.

### Compound 322

¹H NMR (400 MHz, DMSO-d₆, ppm) δ: 12.83 (s, 1H), 8.78 (s, 2H), 8.64 (s, 1H), 5.25-5.11 (m, 1H), 3.78-3.65 (m, 7H), 3.53-3.40 (m, 5H), 2.45-2.40 (m, 2H), 1.49 (d, J=6.8 Hz, 3H).

LCMS (ESI) calcd. for C₂₁H₂₂F₃N₉O₃[M+H]⁺ m/z 506.18, found 506.25.

### 26. Synthesis of 1-(4-(2-oxo-2-(4-(5-(trifluoromethyl)pyrimidin-2-yl)piperazin-1-yl)ethoxy)butan-2-yl)-3-(trifluoromethyl)-1,5-dihydro-4H-pyrazolo[3,4-d]pyridazin-4-one (compounds 327 and 328)

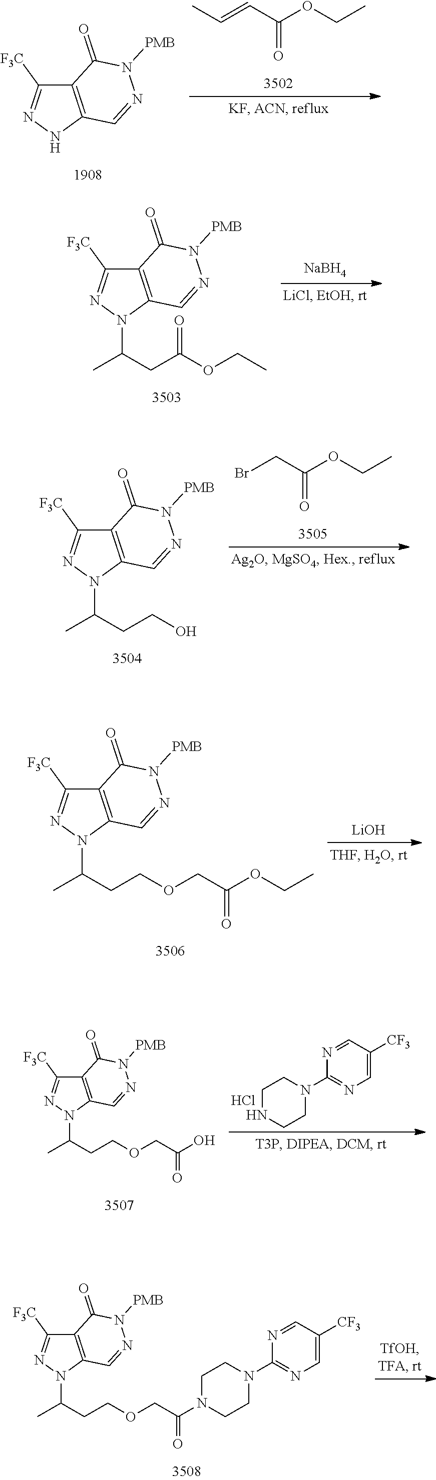

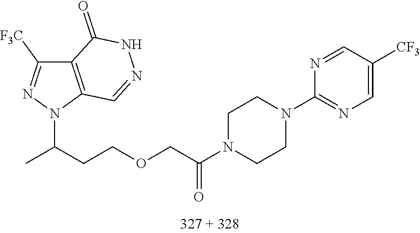 [SMILES: CC(CCOCC(=O)N1CCN(c2ncc(C(F)(F)F)cn2)CC1)n1nc(C(F)(F)F)c2c(=O)[nH]ncc21]

### Preparation of ethyl 3-(5-(4-methoxybenzyl)-4-oxo-3-(trifluoromethyl)-4,5-dihydro-1H-pyrazolo[3,4-d]pyridazin-1-yl)butanoate (3503)

To a solution of 5-(4-methoxybenzyl)-3-(trifluoromethyl)-1,5-dihydro-4H-pyrazolo[3,4-d]pyridazin-4-one 1908 (1.0 g, 0.0030 mol) in MeCN (50 mL) were added ethyl (E)-but-2-enoate 3502 (0.99 g, 0.0086 mol) and KF (0.36 g, 0.0062 mol) at room temperature. The reaction mixture was stirred at 80° C. for 18 h. The reaction solution was quenched with water and extracted with EtOAc (50 mL×3). The organic phases were concentrated under reduced pressure and the residue was purified by flash chromatography (eluting with PE/EtOAc=100:0 to 80:20) to give ethyl 3-(5-(4-methoxybenzyl)-4-oxo-3-(trifluoromethyl)-4,5-dihydro-1H-pyrazolo[3,4-d]pyridazin-1-yl)butanoate 3503 (0.7 g, 70% purity, 35% yield) as a yellow oil.

LCMS (ESI) calcd for C₂₀H₂₁F₃N₄O₄[M+H]⁺ m/z 439.15, found 439.05.

### Preparation of 1-(4-hydroxybutan-2-yl)-5-(4-methoxybenzyl)-3-(trifluoromethyl)-1,5-dihydro-4H-pyrazolo[3,4-d]pyridazin-4-one (3504)

To a solution of ethyl 3-(5-(4-methoxybenzyl)-4-oxo-3-(trifluoromethyl)-4,5-dihydro-1H-pyrazolo[3,4-d]pyridazin-1-yl)butanoate 3503 (1 g, 0.0023 mol) in EtOH (100 mL) were added NaBH₄ (0.35 g, 0.0092 mol) and LiCl (0.39 g, 0.0092 mol) at 0° C. The reaction mixture was stirred at room temperature for 1 h. The reaction solution was quenched with water and extracted with EtOAc (50 mL×3). The combined organic phases were concentrated under reduced pressure and the residue was purified by flash chromatography (eluting with PE/EtOAc=100:0 to 70:30) to give 1-(4-hydroxybutan-2-yl)-5-(4-methoxybenzyl)-3-(trifluoromethyl)-1,5-dihydro-4H-pyrazolo[3,4-d]pyridazin-4-one 3504 (0.68 g, 90% purity, 65% yield) as a yellow solid.

LCMS (ESI) calcd for C₁₈H₁₉F₃N₄O₃[M+H]⁺ m/z 397.14, found 397.25.

### Preparation of ethyl 2-(3-(5-(4-methoxybenzyl)-4-oxo-3-(trifluoromethyl)-4,5-dihydro-1H-pyrazolo[3,4-d]pyridazin-1-yl)butoxy)acetate (3506)

To a solution of 1-(4-hydroxybutan-2-yl)-5-(4-methoxybenzyl)-3-(trifluoromethyl)-1,5-dihydro-4H-pyrazolo[3,4-d]pyridazin-4-one 3504 (680 mg, 1.72 mmol) in hexane (50 mL) were added ethyl 2-bromoacetate 3505 (2.86 g, 17.2 mmol), Ag₂O (3.18 g, 13.72 mmol) and MgSO₄ (0.823 g, 6.86 mmol) at room temperature. The reaction mixture was stirred at 80° C. for 18 h. The resulting solution was filtered through celite and the filter cake was washed with DCM (5 mL×4). The filtrate was concentrated under reduced pressure and the residue was purified by flash chromatography (eluting with PE/EtOAc=100:0 to 70:30) to give ethyl 2-(3-(5-(4-methoxybenzyl)-4-oxo-3-(trifluoromethyl)-4,5-dihydro-1H-pyrazolo[3,4-d]pyridazin-1-yl)butoxy)acetate 3506 (520 mg, 70% purity, 43% yield) as a yellow oil.

LCMS (ESI) calcd for C₂₂H₂₅F₃N₄O₅[M+H]⁺ m/z 483.15, found 483.35.

### Preparation of 2-(3-(5-(4-methoxybenzyl)-4-oxo-3-(trifluoromethyl)-4,5-dihydro-1H-pyrazolo[3,4-d]pyridazin-1-yl)butoxy)acetic acid (3507)

To a solution of ethyl 2-(3-(5-(4-methoxybenzyl)-4-oxo-3-(trifluoromethyl)-4,5-dihydro-1H-pyrazolo[3,4-d]pyridazin-1-yl)butoxy)acetate 3506 (500 mg, 1.036 mmol) in THF/H₂O (3:1, 30 mL) was added LiOH (25 mg, 1.03 mmol) at room temperature. The reaction mixture was stirred at room temperature for 1 h. The reaction solution was adjusted pH to 4 with 1 M aqueous HCl. The water phase was on a Biotage Isolera One (C₁₈ column, eluting with 60% to 90% MeCN/H₂O containing 0.1% formic acid) to obtain 2-(3-(5-(4-methoxybenzyl)-4-oxo-3-(trifluoromethyl)-4,5-dihydro-1H-pyrazolo[3,4-d]pyridazin-1-yl)butoxy)acetic acid 3507 (300 mg, 50% purity, 31% yield) as a white solid.

LCMS (ESI) calcd for C₂₀H₂₁F₃N₄O₅[M+H]⁺ m/z 455.15, found 455.25.

### Preparation of 5-(4-methoxybenzyl)-1-(4-(2-oxo-2-(4-(5-(trifluoromethyl)pyrimidin-2-yl)piperazin-1-yl)ethoxy)butan-2-yl)-3-(trifluoromethyl)-1,5-dihydro-4H-pyrazolo[3,4-d]pyridazin-4-one (3508)

To a solution of 2-(3-(5-(4-methoxybenzyl)-4-oxo-3-(trifluoromethyl)-4,5-dihydro-1H-pyrazolo[3,4-d]pyridazin-1-yl)butoxy)acetic acid 3507 (300 mg, 0.33 mmol) in DCM (10 mL) were added 2-(piperazin-1-yl)-5-(trifluoromethyl)pyrimidine hydrochloride (92 mg, 0.40 mmol), T3P (50% wt in EtOAc, 420 mg, 0.66 mmol), DIPEA (128 mg, 0.99 mmol) at room temperature successively. The mixture was kept stirring at room temperature for 1 h. The resulting mixture was diluted with water and extracted with DCM (20 mL×3). The combined organic layer was dried over Na₂SO₄ and concentrated under reduced pressure. The residue was purified on a Biotage Isolera One (C₁₈ column, eluting with 60% to 90% MeCN/H₂O containing 0.1% formic acid) to give 5-(4-methoxybenzyl)-1-(4-(2-oxo-2-(4-(5-(trifluoromethyl)pyrimidin-2-yl)piperazin-1-yl)ethoxy)butan-2-yl)-3-(trifluoromethyl)-1,5-dihydro-4H-pyrazolo[3,4-d]pyridazin-4-one 3508 (110 mg, 80% purity, 39% yield) as a white solid.

LCMS (ESI) calcd for C₂₉H₃₀F₆N₈O₄[M+H]⁺ m/z 669.23, found 669.20.

### Preparation of 1-(4-(2-oxo-2-(4-(5-(trifluoromethyl)pyrimidin-2-yl)piperazin-1-yl)ethoxy)butan-2-yl)-3-(trifluoromethyl)-1,5-dihydro-4H-pyrazolo[3,4-d]pyridazin-4-one (mixture of compounds 327 and 328)

To a solution of 5-(4-methoxybenzyl)-1-(4-(2-oxo-2-(4-(5-(trifluoromethyl)pyrimidin-2-yl)piperazin-1-yl)ethoxy)butan-2-yl)-3-(trifluoromethyl)-1,5-dihydro-4H-pyrazolo[3,4-d]pyridazin-4-one 3508 (110 mg, 0.16 mmol) in TFA (5 mL) was added TfOH (123 mg, 0.82 mmol) at room temperature. The reaction mixture was stirred at room temperature for 0.5 h. The reaction solution was adjusted pH to 7-8 with saturated aqueous NaHCO₃ at 0° C. The solution was extracted with EtOAc. The combined organic phases were concentrated and purified on a Biotage Isolera One (C18 column, eluting with 60% to 90% MeCN/H₂O containing 0.1% formic acid) to provide 1-(4-(2-oxo-2-(4-(5-(trifluoromethyl)pyrimidin-2-yl)piperazin-1-yl)ethoxy)butan-2-yl)-3-(trifluoromethyl)-1,5-dihydro-4H-pyrazolo[3,4-d]pyridazin-4-one 327 and 328 (60 mg, 95% purity, 63% yield) as a white solid.

LCMS (ESI) calcd for C₂₁H₂₂F₆N₈O₃[M+H]⁺ m/z 549.17, found 549.10.

### Chiral resolution of 1-(4-(2-oxo-2-(4-(5-(trifluoromethyl)pyrimidin-2-yl)piperazin-1-yl)ethoxy)butan-2-yl)-3-(trifluoromethyl)-1,5-dihydro-4H-pyrazolo[3,4-d]pyridazin-4-one (compounds 327 and 328)

Compounds 327 and 328 were separated by SFC (Column: CHIRALPAK OJ-H, 250 mm×20 mm I.D., 5 mm; Mobile phase: CO₂/IPA=85/15) and concentrated under reduced pressure to afford the first fraction as 327 (20.2 mg, 100% purity, 100% ee, white solid) and the second fraction as 328 (17.9 mg, 99% purity, 95% ee, white solid).

### Compound 327

¹H NMR (400 MHz, DMSO-d₆, ppm) δ:12.85 (s, 1H), 8.73 (s, 2H), 8.67 (s, 1H), 5.13-5.15 (m, 1H), 4.07 (s, 2H), 3.86-3.76 (m, 4H), 3.56-3.47 (m, 2H), 3.45-3.37 (m, 3H), 3.18-3.08 (m, 1H), 2.19-2.10 (m, 2H), 1.55 (d, J=6.8 Hz, 3H).

LCMS (ESI) calcd for C₂₁H₂₂F₆N₈O₃[M+H]⁺ m/z 549.17, found 549.30.

### Compound 328

¹H NMR (400 MHz, DMSO-d₆, ppm) δ:12.85 (s, 1H), 8.73 (s, 2H), 8.67 (s, 1H), 5.10-5.15 (m, 1H), 4.07 (s, 2H), 3.87-3.73 (m, 4H), 3.56-3.46 (m, 2H), 3.45-3.37 (m, 3H), 3.17-3.09 (m, 1H), 2.17-2.09 (m, 2H), 1.55 (d, J=6.8 Hz, 3H).

LCMS (ESI) calcd for C₂₁H₂₂F₆N₈O₃[M+H]⁺ m/z 549.17, found 549.30.

### 27. Synthesis of 3-(difluoromethyl)-1-(1-(3-oxo-3-(4-(5-(trifluoromethyl)pyrimidin-2-yl)piperazin-1-yl)propoxy)propan-2-yl)-1,5-dihydro-4H-pyrrolo[2,3-d]pyridazin-4-one (compounds 395 and 396)

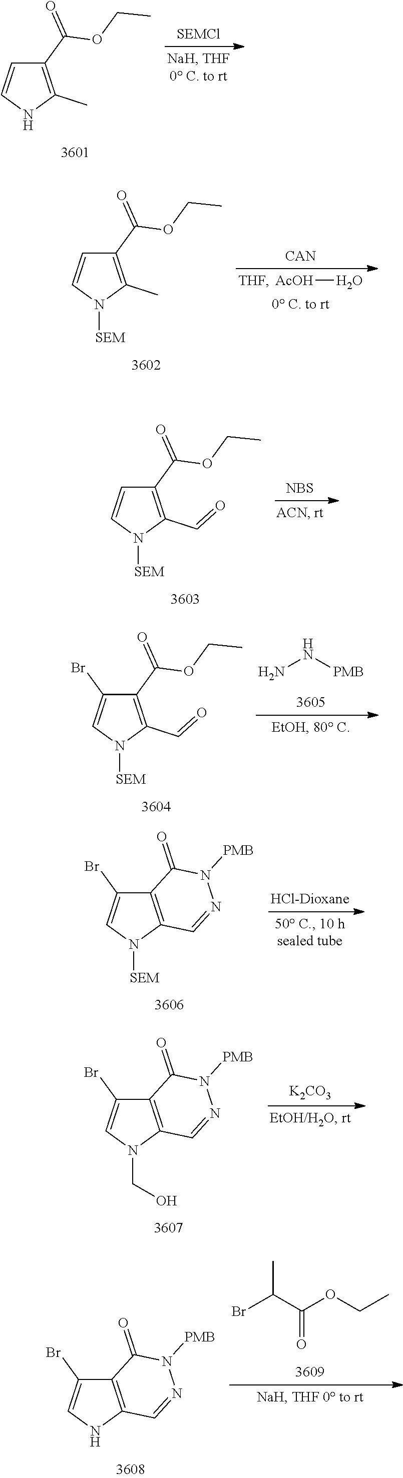

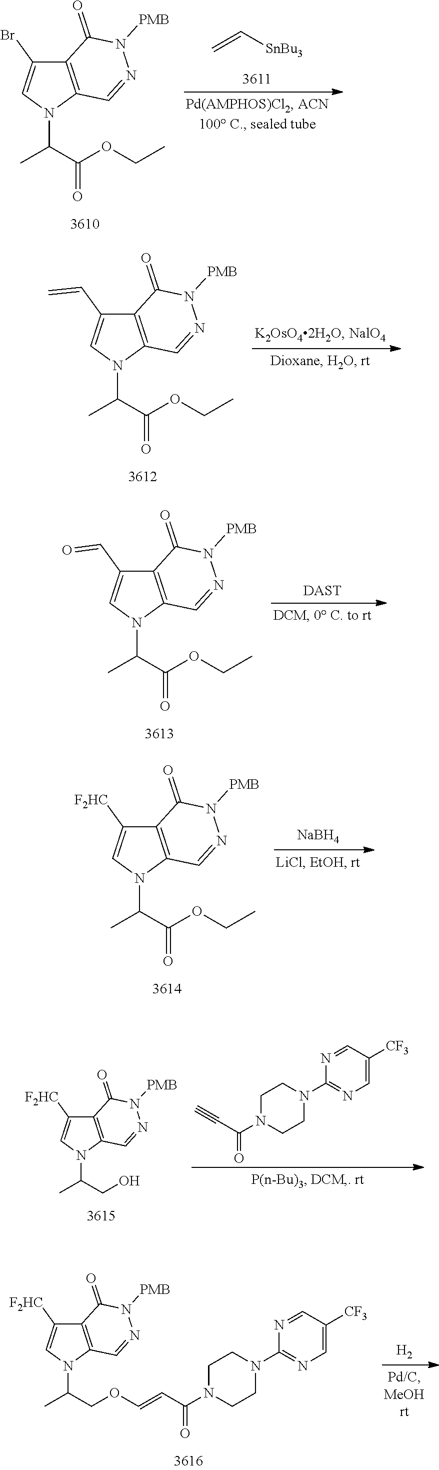

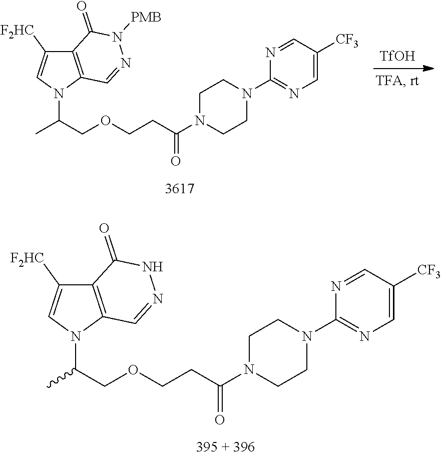

### Preparation of ethyl 2-methyl-1-((2-(trimethylsilyl)ethoxy)methyl)-JH-pyrrole-3-carboxylate (3602)

To a solution of ethyl 2-methyl-1H-pyrrole-3-carboxylate 3601 (45 g, 293.8 mmol) in THF (1000 mL) was added NaH (23.5 g, 587.6 mmol, 60% wt) at 00° C. The reaction mixture was stirred at 0° C. for 10 min SEMCl (58.8 g, 352.5 mmol) was added dropwise at 0° C. The reaction mixture was stirred at room temperature for 5 h. The reaction mixture was quenched with cold water and then extracted with EtOAc (300 mL×3). The combined organic layers were concentrated under reduced pressure. The residue was purified by flash column chromatography (eluting with PE/EtOAc=85:15 to 70:30) to afford ethyl 2-methyl-1-((2-(trimethylsilyl)ethoxy)methyl)-1H-pyrrole-3-carboxylate 3602 (52 g, 90% purity, 56% yield) as a yellow oil.

LCMS (ESI) calcd for Cl₄H₂₅NO₃Si [M+H]⁺ m/z 284.16, found 284.25.

### Preparation of ethyl 2-formyl-1-((2-(trimethylsilyl)ethoxy)methyl)-1H-pyrrole-3-carboxylate (3603)

To a solution of ethyl 2-methyl-1-((2-(trimethylsilyl)ethoxy)methyl)-1H-pyrrole-3-carboxylate 3602 (50 g, 175.8 mmol) in THF/AcOH/H₂O (800 mL, 1:1:1) was added CAN (385.5 g, 703.2 mmol) at 0° C. The reaction mixture was stirred at room temperature for 1 h. The reaction mixture was poured into ice-water (500 mL) and stirred for another 30 min. The resulting solution was extracted with EtOAc (300 mL×3). The combined organic layer was washed with brine, dried over Na₂SO₄ and concentrated under reduced pressure. The residue was purified by flash column chromatography (eluting with PE/EtOAc=100:0 to 80:20) to obtain ethyl 2-formyl-1-((2-(trimethylsilyl)ethoxy)methyl)-1H-pyrrole-3-carboxylate 3603 (18 g, 90% purity, 31% yield) as a yellow oil.

LCMS (ESI) calcd for C₁₄H₂₃NO₄Si [M+H]⁺ m/z 298.14, found 298.18.

### Preparation of ethyl 4-bromo-2-formyl-1-((2-(trimethylsilyl)ethoxy)methyl)-1H-pyrrole-3-carboxylate (3604)

To a solution of ethyl 2-formyl-1-((2-(trimethylsilyl)ethoxy)methyl)-1H-pyrrole-3-carboxylate 3603 (18 g, 60.3 mmol) in ACN (300 mL) was added NBS (10.7 g, 60.3 mmol) at rt. The reaction mixture was stirred at rt for 2 h. The reaction mixture was concentrated under reduced pressure. The residue was purified by flash column chromatography (eluting with PE/EtOAc=85:15 to 70:30) to afford ethyl 4-bromo-2-formyl-1-((2-(trimethylsilyl)ethoxy)methyl)-1H-pyrrole-3-carboxylate 3604 (15 g, 90% purity, 59% yield) as a yellow oil.

¹H NMR (400 MHz, DMSO-d₆, ppm) δ: 10.12 (s, 1H), 7.78 (s, 1H), 5.68 (s, 2H), 4.44-4.33 (m, 2H), 3.58-3.53 (m, 2H), 1.38 (t, J=7.2 Hz, 3H), 0.88 (t, J=7.8 Hz, 2H), 0.00 (s, 9H).

### Preparation of 3-bromo-5-(4-methoxybenzyl)-1-((2-(trimethylsilyl)ethoxy)methyl)-1,5-dihydro-4H-pyrrolo[2,3-d]pyridazin-4-one (3606)

To a solution of ethyl 4-bromo-2-formyl-1-((2-(trimethylsilyl)ethoxy)methyl)-1H-pyrrole-3-carboxylate 3604 (10 g, 26.5 mmol) in EtOH (50 mL) was added (2-methoxy-5-methylphenyl) hydrazine 3605 (8.1 g, 53 mmol) at rt. The reaction mixture was stirred at 80° C. for 1 h. The reaction solution was concentrated under reduced pressure. The residue was purified by flash column chromatography (eluting with PE/EtOAc=85:15 to 20:80) to afford 3-bromo-5-(4-methoxybenzyl)-1-((2-(trimethylsilyl)ethoxy)methyl)-1,5-dihydro-4H-pyrrolo[2,3-d]pyridazin-4-one 3606 (8.3 g, 90% purity, 60% yield) as a yellow solid.

LCMS (ESI) calcd for C₂₀H₂₆BrN₃O₃Si [M+H]⁺ m/z 464.09, found 464.18.

### Preparation of 3-bromo-5-(4-methoxybenzyl)-1,5-dihydro-4H-pyrrolo[2,3-d]pyridazin-4-one (3608)

A solution of 3-bromo-5-(4-methoxybenzyl)-1-((2-(trimethylsilyl)ethoxy)methyl)-1,5-dihydro-4H-pyrrolo[2,3-d]pyridazin-4-one 3606 (8.2 g, 17.6 mmol) in HCl-dioxane (150 mL, 4 M) was prepared at rt. The reaction mixture was stirred at 50° C. for 16 h in a sealed tube. After LCMS showed 3607 was formed the reaction solution was concentrated under reduced pressure. The residue was dissolved in EtOH/H₂O (100 mL, 5:1), K₂CO₃ (24.3 g, 0.18 mmol) was added at rt. The reaction mixture was stirred at rt for 2 h. The reaction solution was diluted with H₂O (200 mL), the aqueous layer was extracted with EtOAc (100 mL×3). The combined organic layers were concentrated under reduced pressure to give 3-bromo-5-(4-methoxybenzyl)-1,5-dihydro-4H-pyrrolo[2,3-d]pyridazin-4-one 3608 (5.8 g, 80% purity, 79% yield) as a yellow solid.

LCMS (ESI) calcd for C₁₄H₁₂BrN₃O₂ [M+H]⁺ m/z 334.01, found 334.07.

### Preparation of ethyl 2-(3-bromo-5-(4-methoxybenzyl)-4-oxo-4,5-dihydro-1H-pyrrolo[2,3-d]pyridazin-1-yl)propanoate (3610)

To a solution of 3-bromo-5-(4-methoxybenzyl)-1,5-dihydro-4H-pyrrolo[2,3-d]pyridazin-4-one 3608 (6 g, 18 mmol) in THE (100 mL) was added NaH (2.2 g, 54 mmol, 60% wt) at 0° C. The reaction mixture was stirred at 0° C. for 10 min. Ethyl 2-bromopropanoate 3609 (4.9 g, 27 mmol) was added dropwise at 0° C. The reaction mixture was stirred at room temperature for 12 h. The reaction mixture was quenched with cold water and then extracted with EtOAc (100 mL×3). The combined organic layers were concentrated under reduced pressure. The residue was purified by flash column chromatography (eluting with PE/EtOAc=85:15 to 60:40) to afford ethyl 2-(3-bromo-5-(4-methoxybenzyl)-4-oxo-4,5-dihydro-1H-pyrrolo[2,3-d]pyridazin-1-yl)propanoate 3610 (3.0 g, 90% purity, 34% yield) as a yellow solid.

LCMS (ESI) calcd for C₁₉H₂₀BrN₃O₄[M+H]⁺ m/z 434.06, found 434.15.

### Preparation of ethyl 2-(5-(4-methoxybenzyl)-4-oxo-3-vinyl-4,5-dihydro-1H-pyrrolo[2,3-d]pyridazin-1-yl)propanoate (3612)

To a solution of ethyl 2-(3-bromo-5-(4-methoxybenzyl)-4-oxo-4,5-dihydro-1H-pyrrolo[2,3-d]pyridazin-1-yl)propanoate 3610 (4.0 g, 9.2 mmol) in ACN (30 mL) were added tributyl(vinyl)stannane 3611 (5.83 g, 18.4 mmol) and Pd(AMPHOS)Cl₂ (650 mg, 0.9 mmol) at rt. The resulting mixture was stirred at 100° C. for 1 h in a sealed tube. After cooling to rt, the mixture was concentrated in vacuo. The residue was purified by flash chromatography (eluting with PE/EtOAc=100:0 to 60:40) to obtain ethyl 2-(5-(4-methoxybenzyl)-4-oxo-3-vinyl-4,5-dihydro-1H-pyrrolo[2,3-d]pyridazin-1-yl)propanoate 3612 (2.96 g, 90% purity, 76% yield) as a white solid.

LCMS (ESI) calcd for C₂₁H₂₃N₃O₄ [M+H]⁺ m/z 382.17, found 382.19.

### Preparation of ethyl 2-(3-formyl-5-(4-methoxybenzyl)-4-oxo-4,5-dihydro-1H-pyrrolo[2,3-d]pyridazin-1-yl)propanoate (3613)

To a solution of ethyl 2-(5-(4-methoxybenzyl)-4-oxo-3-vinyl-4,5-dihydro-1H-pyrrolo[2,3-d]pyridazin-1-yl)propanoate 3612 (3 g, 7.9 mmol) in 1,4-dioxane/H₂O (2:1, 50 mL) were added K₂OsO₄·2H₂O (290 mg, 0.8 mmol) and NaIO₄ (6.76 g, 31.6 mmol) at rt. The reaction mixture was stirred at rt for 5 h. The resulting mixture was diluted with water (50 mL) and extracted with EtOAc (100 mL×3). The combined organic layer was washed with brine and concentrated under reduced pressure. The residue was purified by flash chromatography (eluting with PE/EtOAc=100:0 to 50:50) to give ethyl 2-(3-formyl-5-(4-methoxybenzyl)-4-oxo-4,5-dihydro-1H-pyrrolo[2,3-d]pyridazin-1-yl)propanoate 3613 (1.7 g, 90% purity, 51% yield) as a yellow oil.

LCMS (ESI) calcd for C₂₀H₂₁N₃O₅ [M+H]⁺ m/z 384.15, found 384.19.

### Preparation of ethyl 2-(3-(difluoromethyl)-5-(4-methoxybenzyl)-4-oxo-4,5-dihydro-1H-pyrrolo[2,3-d]pyridazin-1-yl)propanoate (3614)

To a solution of ethyl 2-(3-formyl-5-(4-methoxybenzyl)-4-oxo-4,5-dihydro-1H-pyrrolo[2,3-d]pyridazin-1-yl)propanoate 3613 (1.7 g, 4.4 mmol) in DCM (50 mL) was added DAST (7.1 g, 44.0 mmol) at 0° C. The reaction mixture was stirred at rt for 16 h. The reaction solution was adjusted to pH 8-9 with saturated aq. NaHCO₃ at 0° C. The aqueous layer was extracted with EtOAc (50 mL×3). The combined organic layers were concentrated under reduced pressure. The residue was purified by flash column chromatography (eluting with PE/EtOAc=85:15 to 70:30) to afford ethyl 2-(3-(difluoromethyl)-5-(4-methoxybenzyl)-4-oxo-4,5-dihydro-1H-pyrrolo[2,3-d]pyridazin-1-yl)propanoate 3614 (1.36 g, 90% purity, 68% yield) as a white solid.

LCMS (ESI) calcd for C₂₀H₂₁F₂N₃O₄[M+H]⁺ m/z 406.15, found 406.14.

### Preparation of 3-(difluoromethyl)-1-(1-hydroxypropan-2-yl)-5-(4-methoxybenzyl)-1,5-dihydro-4H-pyrrolo[2,3-d]pyridazin-4-one (3615)

To a solution of ethyl 2-(3-(difluoromethyl)-5-(4-methoxybenzyl)-4-oxo-4,5-dihydro-1H-pyrrolo[2,3-d]pyridazin-1-yl)propanoate 3614 (1.35 g, 3.3 mmol) in EtOH (30 mL) was added NaBH₄ (0.5 g, 13.2 mmol) and LiCl (0.56 g, 13.2 mmol) successively at rt. The reaction was stirred at rt for 2 h. The reaction was quenched with H₂O (30 mL) and extracted with EtOAc (30 mL×2). The organic layer was concentrated under reduced pressure. The residue was purified by flash column chromatography (eluting with PE/EtOAc=70:30 to 20:80) to afford 3-(difluoromethyl)-1-(1-hydroxypropan-2-yl)-5-(4-methoxybenzyl)-1,5-dihydro-4H-pyrrolo[2,3-d]pyridazin-4-one 3615 (740 mg, 90% purity, 55% yield) as a white solid.

LCMS (ESI) calcd for C₁₈H₁₉F₂N₃O₃[M+H]⁺ m/z 364.14, found 364.18.

### Preparation of (E)-3-(difluoromethyl)-5-(4-methoxybenzyl)-1-(1-((3-oxo-3-(4-(5-(trifluoromethyl)pyrimidin-2-yl)piperazin-1-yl)prop-1-en-1-yl)oxy)propan-2-yl)-1,5-dihydro-4H-pyrrolo[2,3-d]pyridazin-4-one (3616)

To the solution of 3-(difluoromethyl)-1-(1-hydroxypropan-2-yl)-5-(4-methoxybenzyl)-1,5-dihydro-4H-pyrrolo[2,3-d]pyridazin-4-one 3615 (70 mg, 0.19 mmol) in DCM (5 mL) were added 1-(4-(5-(trifluoromethyl)pyrimidin-2-yl)piperazin-1-yl)prop-2-yn-1-one (66 mg, 0.23 mmol) and P(n-Bu)₃ (19 mg, 0.10 mmol) at rt. The reaction mixture was stirred at rt for 2 h. The reaction mixture was diluted with DCM and water. The aqueous layer was extracted with DCM (20 mL×3). The combined organic layers were dried over Na₂SO₄ and concentrated under reduced pressure. The residue was purified by flash column chromatography (eluting with PE/EtOAc=20:80 to 0:100) to afford (E)-3-(difluoromethyl)-5-(4-methoxybenzyl)-1-(1-((3-oxo-3-(4-(5-(trifluoromethyl)pyrimidin-2-yl)piperazin-1-yl)prop-1-en-1-yl)oxy)propan-2-yl)-1,5-dihydro-4H-pyrrolo[2,3-d]pyridazin-4-one 3616 (135 mg, 90% purity, 97% yield) as a white solid.

LCMS (ESI) calcd for C₃₀H₃₀F₅N₇O₄[M+H]⁺ m/z 648.23, found 648.37.

### Preparation of 3-(difluoromethyl)-5-(4-methoxybenzyl)-1-(1-(3-oxo-3-(4-(5-(trifluoromethyl)pyrimidin-2-yl)piperazin-1-yl)propoxy)propan-2-yl)-1,5-dihydro-4H-pyrrolo[2,3-d]pyridazin-4-one (3617)

A solution of (E)-3-(difluoromethyl)-5-(4-methoxybenzyl)-1-(1-((3-oxo-3-(4-(5-(trifluoromethyl)pyrimidin-2-yl)piperazin-1-yl)prop-1-en-1-yl)oxy)propan-2-yl)-1,5-dihydro-4H-pyrrolo[2,3-d]pyridazin-4-one 3616 (130 mg, 0.20 mmol) and Pd/C (15 mg) in MeOH (10 mL) was stirred at rt for 2 h under H₂ atmosphere. The resulting solution was filtered through diatomaceous earth and the filter cake was washed with DCM (5 mL×4). The filtrate was concentrated under reduced pressure to give 3-(difluoromethyl)-5-(4-methoxybenzyl)-1-(1-(3-oxo-3-(4-(5-(trifluoromethyl)pyrimidin-2-yl)piperazin-1-yl)propoxy)propan-2-yl)-1,5-dihydro-4H-pyrrolo[2,3-d]pyridazin-4-one 3617 (130 mg, 90% purity, 90% yield) as a white solid.

LCMS (ESI) calcd for C₃₀H₃₂F₅N₇O₄[M+H]⁺ m/z 650.24, found 650.30.

### Preparation of 3-(difluoromethyl)-1-(1-(3-oxo-3-(4-(5-(trifluoromethyl)pyrimidin-2-yl)piperazin-1-yl)propoxy)propan-2-yl)-1,5-dihydro-4H-pyrrolo[2,3-d]pyridazin-4-one (mixture of 395 and 396)

To a solution of 3-(difluoromethyl)-5-(4-methoxybenzyl)-1-(1-(3-oxo-3-(4-(5-(trifluoromethyl)pyrimidin-2-yl)piperazin-1-yl)propoxy)propan-2-yl)-1,5-dihydro-4H-pyrrolo[2,3-d]pyridazin-4-one 3617 (105 mg, 0.16 mmol) in TFA (3 mL) was added TfOH (0.3 mL) at 0° C. The reaction solution was stirred at rt for 1 h. The mixture was adjusted to pH 8-9 with saturated aqueous NaHCO₃ at 0° C., then extracted with EtOAc (10 mL×3). The combined organic layers were concentrated under reduced pressure. The residue was purified by C18 column (mobile phase: ACN-H₂O (0.1% FA), gradient: 10-95) to give 3-(difluoromethyl)-1-(1-(3-oxo-3-(4-(5-(trifluoromethyl)pyrimidin-2-yl)piperazin-1-yl)propoxy)propan-2-yl)-1,5-dihydro-4H-pyrrolo[2,3-d]pyridazin-4-one (mixture of 395 and 396) as a white solid.

### Chiral resolution of 3-(difluoromethyl)-1-(1-(3-oxo-3-(4-(5-(trifluoromethyl)pyrimidin-2-yl)piperazin-1-yl)propoxy)propan-2-yl)-1,5-dihydro-4H-pyrrolo[2,3-d]pyridazin-4-one (compounds 395 and 396)

Compounds 395 and 396 were separated by SFC (Column: DAICEL OD-H 4.6 mm I.D.×250 mmL 5 m; Mobile phase: CO₂/IPA [0.1% NH₃ (7 M Solution in MeOH)]=75/25) and concentrated under reduced pressure to afford the first fraction as 395 (24.3 mg, 100% purity, 99% ee, white solid) and the second fraction as 396 (20.3 mg, 100% purity, 98% ee, white solid).

### Compound 395

¹H NMR (400 MHz, DMSO-d₆, ppm) δ: 12.48 (s, 1H), 8.73 (s, 2H), 8.46 (s, 1H), 7.95 (s, 1H), 7.27 (t, J=55.6 Hz, 1H), 4.97-4.84 (m, 1H), 3.82-3.73 (m, 4H), 3.73-3.63 (m, 3H), 3.62-3.54 (m, 1H), 3.53-3.41 (m, 4H), 2.49-2.45 (m, 2H), 1.46 (d, J=6.8 Hz, 3H).

LCMS (ESI) calcd for C₂₂H₂₄F₅N₇O₃[M+H]⁺ m/z 530.19, found 530.30.

### Compound 396

¹H NMR (400 MHz, DMSO-d₆, ppm) δ: 12.49 (s, 1H), 8.73 (s, 2H), 8.46 (s, 1H), 7.95 (s, 1H), 7.27 (t, J=55.6 Hz, 1H), 4.98-4.84 (m, 1H), 3.82-3.63 (m, 7H), 3.64-3.54 (m, 1H), 3.53-3.40 (m, 4H), 2.50-2.45 (m, 2H), 1.46 (d, J=6.8 Hz, 3H).

LCMS (ESI) calcd for C₂₂H₂₄F₅N₇O₃[M+H]⁺ m/z 530.19, found 530.30.

### 28. Synthesis of 2-(4-(3-(2-(4-oxo-3-(trifluoromethyl)-4,5-dihydro-1H-pyrazolo[3,4-d]pyridazin-1-yl)propoxy-1,1-d2)propanoyl)piperazin-1-yl)pyrimidine-5-carbonitrile (compounds 420 and 421)

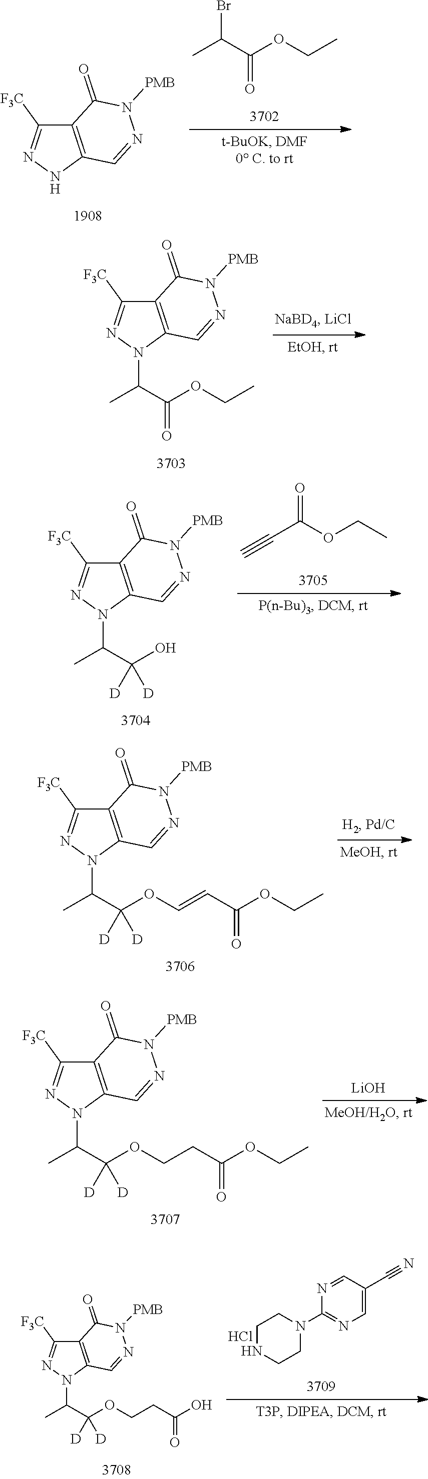

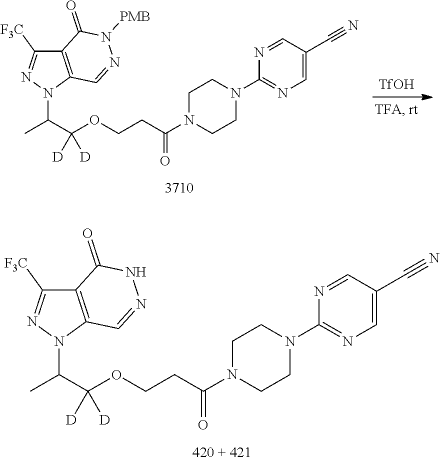

### Preparation of ethyl 2-(5-(4-methoxybenzyl)-4-oxo-3-(trifluoromethyl)-4,5-dihydro-1H-pyrazolo[3,4-d]pyridazin-1-yl)propanoate (3703)

To a solution of 5-(4-methoxybenzyl)-3-(trifluoromethyl)-1,5-dihydro-4H-pyrazolo[3,4-d]pyridazin-4-one 1908 (1 g, 0.0031 mol), ethyl 2-bromopropanoate 3702 (1.68 g, 0.0093 mmol) in DMF (30 mL) was added t-BuOK (1.04 mg, 0.0093 mol) at 0° C. The reaction mixture was stirred at rt for 6 h. The reaction solution was quenched with water and extracted with EtOAc (100 mL×3). The combined organic phases were concentrated under reduced pressure. The residue was purified by silica gel column (eluting with EtOAc/PE, 0 to 50%) to obtain ethyl 2-(5-(4-methoxybenzyl)-4-oxo-3-(trifluoromethyl)-4,5-dihydro-1H-pyrazolo[3,4-d]pyridazin-1-yl)propanoate 3703 (1.2 g, 90% purity, 80% yield) as a white solid.

LCMS (ESI) calcd for C₁₉H₁₉F₃N₄O₄[M+H]⁺ m/z 425.14, found 425.05.

### Preparation of 1-(1-hydroxypropan-2-yl-1,1-d2)-5-(4-methoxybenzyl)-3-(trifluoromethyl)-1,5-dihydro-4H-pyrazolo[3,4-d]pyridazin-4-one (3704)

To a solution of ethyl 2-(5-(4-methoxybenzyl)-4-oxo-3-(trifluoromethyl)-4,5-dihydro-1H-pyrazolo[3,4-d]pyridazin-1-yl)propanoate 3703 (1.2 g, 0.0028 mol) in EtOH (30 mL) were added LiCl (0.47 g, 0.0112 mmol) and NaBD₄ (0.47 g, 0.0112 mol) at rt. The reaction mixture was stirred at rt for 2 h. The resulting mixture was quenched with water and extracted with EtOAc (50 mL×3). The combined organic layers were washed with brine and concentrated under reduced pressure. The residue was purified by silica gel column chromatography (eluting with EtOAc/PE, 0 to 80%) to obtain 1-(1-hydroxypropan-2-yl-1,1-d2)-5-(4-methoxybenzyl)-3-(trifluoromethyl)-1,5-dihydro-4H-pyrazolo[3,4-d]pyridazin-4-one 3704 (0.8 g, 90% purity, 67% yield) as white solid.

LCMS (ESI) calcd for C₁₇H₁₅D₂F₃N₄O₃ [M+H]⁺ m/z 385.14, found 385.25.

### Preparation of ethyl (E)-3-(2-(5-(4-methoxybenzyl)-4-oxo-3-(trifluoromethyl)-4,5-dihydro-1H-pyrazolo[3,4-d]pyridazin-1-yl)propoxy-1,1-d2)acrylate (3706)

To a solution of 1-(1-hydroxypropan-2-yl-1,1-d2)-5-(4-methoxybenzyl)-3-(trifluoromethyl)-1,5-dihydro-4H-pyrazolo[3,4-d]pyridazin-4-one 3704 (500 mg, 1.3 mmol), ethyl propiolate 3705 (383 mg, 3.9 mmol) in DCM (10 mL) was added P(n-Bu)₃ (132 mg, 0.65 mmol) at rt. The reaction mixture was stirred at rt for 0.5 h. The resulting mixture was quenched with water and extracted with DCM (50 mL×3). The combined organic layer was washed with brine and concentrated under reduced pressure. The residue was purified by silica gel column (eluting with EtOAc/PE, 0 to 100%) to obtain ethyl (E)-3-(2-(5-(4-methoxybenzyl)-4-oxo-3-(trifluoromethyl)-4,5-dihydro-1H-pyrazolo[3,4-d]pyridazin-1-yl)propoxy-1,1-d2)acrylate 3706 (600 mg, 90% purity, 86% yield) as a yellow oil.

LCMS (ESI) calcd for C₂₂H₂₁D₂F₃N₄O₅ [M+Na]⁺ m/z 505.17, found 505.05.

### Preparation of ethyl 3-(2-(5-(4-methoxybenzyl)-4-oxo-3-(trifluoromethyl)-4,5-dihydro-1H-pyrazolo[3,4-d]pyridazin-1-yl)propoxy-1,1-d2)propanoate (3707)

To a solution of ethyl (E)-3-(2-(5-(4-methoxybenzyl)-4-oxo-3-(trifluoromethyl)-4,5-dihydro-1H-pyrazolo[3,4-d]pyridazin-1-yl)propoxy-1,1-d2)acrylate 3706 (600 mg, 1.24 mmol) in MeOH (20 mL) was added 10% Pd/C (60 mg). The mixture was evacuated and backfilled with hydrogen three times and then charged with hydrogen. The resulting mixture was stirred at room temperature for 2 hours. Then the mixture was filtered through celite and concentrated under vacuum to give crude ethyl 3-(2-(5-(4-methoxybenzyl)-4-oxo-3-(trifluoromethyl)-4,5-dihydro-1H-pyrazolo[3,4-d]pyridazin-1-yl)propoxy-1,1-d2)propanoate 3707 (600 mg, 90% purity, 89% yield) which was used directly in next step without further purification.

LCMS (ESI) calcd for C₂₂H₂₃D₂F₃N₄O₅ [M+H]⁺ m/z 485.19, found 485.15.

### Preparation of 3-(2-(5-(4-methoxybenzyl)-4-oxo-3-(trifluoromethyl)-4,5-dihydro-1H-pyrazolo[3,4-d]pyridazin-1-yl)propoxy-1,1-d2)propanoic acid (3708)

To a solution of ethyl 3-(2-(5-(4-methoxybenzyl)-4-oxo-3-(trifluoromethyl)-4,5-dihydro-1H-pyrazolo[3,4-d]pyridazin-1-yl)propoxy-1,1-d2)propanoate 3707 (600 mg, 1.24 mmol) in MeOH/H₂O (3/1, 30 mL) was added LiOH (89 mg, 3.72 mmol). The mixture was stirred at rt for 2 h. The mixture was acidified with 1 M aqueous HCl to pH 4˜5. The water phase was purified by C₁₈ column (Agela 80 g, mobile phase: ACN-H₂O (0.1% FA), gradient: 20%-50%) to give 3-(2-(5-(4-methoxybenzyl)-4-oxo-3-(trifluoromethyl)-4,5-dihydro-1H-pyrazolo[3,4-d]pyridazin-1-yl)propoxy-1,1-d2)propanoic acid 3708 (400 mg, 90% purity, 63% yield) as a yellow oil.

LCMS (ESI) calcd for C₂₀H₁₉D₂F₃N₄O₅ [M+H]⁺ m/z 457.16, found 457.20.

### Preparation of 2-(4-(3-(2-(5-(4-methoxybenzyl)-4-oxo-3-(trifluoromethyl)-4,5-dihydro-1H-pyrazolo[3,4-d]pyridazin-1-yl)propoxy-1,1-d2)propanoyl)piperazin-1-yl)pyrimidine-5-carbonitrile (3710)

To a solution of 3-(2-(5-(4-methoxybenzyl)-4-oxo-3-(trifluoromethyl)-4,5-dihydro-1H-pyrazolo[3,4-d]pyridazin-1-yl)propoxy-1,1-d2)propanoic acid 3708 (100 mg, 0.22 mmol), 2-(piperazin-1-yl)pyrimidine-5-carbonitrile hydrochloride 3709 (64 mg, 0.28 mmol) and DIPEA (141 mg, 1.1 mmol) in DCM (10 mL) was added T3P (50% in EtOAc, 279 mg, 0.44 mmol) at rt. The mixture was stirred at room temperature for 30 min. The resulting solution was diluted with water (30 mL) and extracted with DCM (30 mL×3). The combined organic layer was dried over Na₂SO₄ and concentrated under reduced pressure. The residue was purified by C₁₈ column (Agela 80 g, mobile phase: ACN-H₂O (0.1% FA), gradient: 20%-80%) to give 2-(4-(3-(2-(5-(4-methoxybenzyl)-4-oxo-3-(trifluoromethyl)-4,5-dihydro-1H-pyrazolo[3,4-d]pyridazin-1-yl)propoxy-1,1-d2)propanoyl)piperazin-1-yl)pyrimidine-5-carbonitrile 3710 (100 mg, 90% purity, 81% yield) as a white solid.

LCMS (ESI) calcd for C₂₉H₂₈D₂F₃N₉O₄ [M+H]⁺ m/z 628.25, found 628.20.

### Preparation of 2-(4-(3-(2-(4-oxo-3-(trifluoromethyl)-4,5-dihydro-1H-pyrazolo[3,4-d]pyridazin-1-yl)propoxy-1,1-d2)propanoyl)piperazin-1-yl)pyrimidine-5-carbonitrile (compounds 420 and 421)

To a solution of 2-(4-(3-(2-(5-(4-methoxybenzyl)-4-oxo-3-(trifluoromethyl)-4,5-dihydro-1H-pyrazolo[3,4-d]pyridazin-1-yl)propoxy-1,1-d2)propanoyl)piperazin-1-yl)pyrimidine-5-carbonitrile 3710 (100 mg, 0.16 mmol) in TFA (5 mL) was added TfOH (0.2 mL) at rt. The reaction mixture was stirred at rt for 0.5 h. The resulting mixture was diluted with DCM (50 mL) and then adjusted pH to 8 with saturated aqueous NaHCO₃ at 0° C. The basified solution was extracted with DCM (20 mL×3). The combined organic layers were washed with brine, dried over Na₂SO₄, and concentrated under reduced pressure. The crude product was purified by C₁₈ column (Agela 40 g, mobile phase: ACN-H₂O (0.1% FA), gradient: 30%-60%) to give 2-(4-(3-(2-(4-oxo-3-(trifluoromethyl)-4,5-dihydro-1H-pyrazolo[3,4-d]pyridazin-1-yl)propoxy-1,1-d2)propanoyl)piperazin-1-yl)pyrimidine-5-carbonitrile (mixture of compounds 420 and 421) (40 mg, 95% purity, 47% yield) as a white solid.

### Chiral resolution of 2-(4-(3-(2-(4-oxo-3-(trifluoromethyl)-4,5-dihydro-1H-pyrazolo[3,4-d]pyridazin-1-yl)propoxy-1,1-d2)propanoyl)piperazin-1-yl)pyrimidine-5-carbonitrile (compounds 420 and 421)

Compounds 420 and 421 were separated by SFC (Column: DAICEL OJ-H 4.6 mm I.D.×250 mmL 5 m; Mobile phase: CO₂/MeOH [0.1% NH₃ (7 M Solution in MeOH)]=65/35) and concentrated under reduced pressure to afford the first fraction as 420 (16.8 mg, 98% purity, 100% ee, white solid) and the second fraction as 421 (14 mg, 99% purity, 100% ee, white solid).

### Compound 420

¹H NMR (400 MHz, DMSO-d₆, ppm) δ:12.83 (s, 1H), 8.78 (s, 2H), 8.65 (s, 1H), 5.16 (q, J=6.8 Hz, 1H), 3.78-3.71 (m, 4H), 3.71-3.63 (m, 1H), 3.55-3.49 (m, 1H), 3.48-3.40 (m, 4H), 2.47-2.40 (m, 2H), 1.49 (d, J=6.8 Hz, 3H).

LCMS (ESI) calcd for C₂₁H₂₀D₂F₃N₉O₃ [M+H]⁺ m/z 508.19, found 508.05.

### Compound 421

¹H NMR (400 MHz, DMSO-d₆, ppm) δ: 12.83 (s, 1H), 8.78 (s, 2H), 8.65 (s, 1H), 5.16 (q, J=6.8 Hz, 1H), 3.78-3.72 (m, 4H), 3.70-3.63 (m, 1H), 3.55-3.49 (m, 1H), 3.48-3.39 (m, 4H), 2.46-2.39 (m, 2H), 1.49 (d, J=6.8 Hz, 3H).

LCMS (ESI) calcd for C₂₁H₂₀D₂F₃N₉O₃ [M+H]⁺ m/z 508.19, found 508.10.

### 29. Synthesis of N-methyl-2-(2-(4-oxo-3-(trifluoromethyl)-4,5-dihydro-1H-pyrazolo[3,4-d]pyridazin-1-yl) propoxy)-N-(1-(5-(trifluoromethyl) pyrimidin-2-yl) piperidin-4-yl) acetamide (compounds 460 and 461)

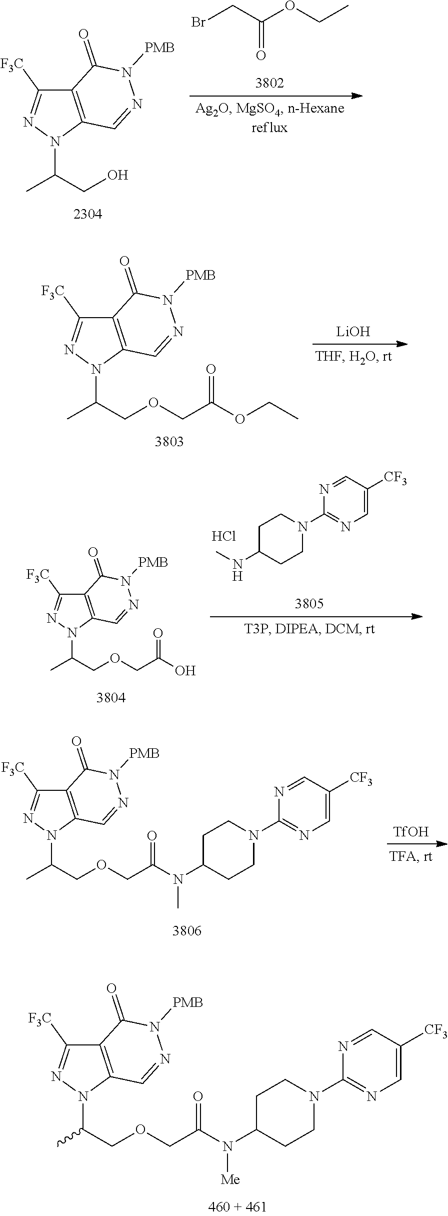

### Preparation of ethyl 2-(2-(5-(4-methoxybenzyl)-4-oxo-3-(trifluoromethyl)-4,5-dihydro-1H-pyrazolo[3,4-d]pyridazin-1-yl) propoxy) acetate (3803)

To a solution of 1-(1-hydroxypropan-2-yl)-5-(4-methoxybenzyl)-3-(trifluoromethyl)-1,5-dihydro-4H-pyrazolo[3,4-d]pyridazin-4-one 2304 (250 mg, 0.65 mmol) in hexane (30 mL) were added ethyl 2-bromoacetate 3802 (765 mg, 4.58 mmol), MgSO₄ (314 mg, 2.62 mmol), Ag₂O (607 mg, 2.62 mmol) at rt successively under an atmosphere of N₂. The mixture was heated at 80° C. for 32 h. The mixture was filtered through celite. The filtrate was concentrated under vacuum, purified by flash chromatography (eluting with PE/EtOAc=100:0 to 60:40) to afford ethyl 2-(2-(5-(4-methoxybenzyl)-4-oxo-3-(trifluoromethyl)-4,5-dihydro-1H-pyrazolo[3,4-d]pyridazin-1-yl)propoxy) acetate 3803 (140 mg, 90% purity, 41% yield) as clear oil.

LCMS (ESI) calcd C₂₁H₂₃F₃N₄O₅ [M+H]+m/z 469.16, found 469.15.

### Preparation of 2-(2-(5-(4-methoxybenzyl)-4-oxo-3-(trifluoromethyl)-4,5-dihydro-1H-pyrazolo[3,4-d]pyridazin-1-yl) propoxy) acetic acid (3804)

To a solution of ethyl 2-(2-(5-(4-methoxybenzyl)-4-oxo-3-(trifluoromethyl)-4,5-dihydro-1H-pyrazolo[3,4-d]pyridazin-1-yl) propoxy) acetate 3803 (190 mg, 0.41 mmol) in THF/H₂O=5:1 (5 mL) was added LiOH (30 mg, 1.22 mmol) at rt. The reaction mixture was stirred at rt for 2 h. The solvent was adjusted to pH 2-3 with saturated NH₄Cl and extracted with DCM. The combined organic phases were washed with water and brine, dried over sodium sulfate, concentrated under vacuum, and purified by C₁₈ column (Agela 40 g, mobile phase: ACN-H₂O (0.1% FA), gradient: 70-80)) to afford 2-(2-(5-(4-methoxybenzyl)-4-oxo-3-(trifluoromethyl)-4,5-dihydro-1H-pyrazolo[3,4-d]pyridazin-1-yl)propoxy)acetic acid 3804 (150 mg, 85% purity, 71% yield) as a clear oil.

LCMS (ESI) calcd C₁₉H₁₉F₃N₄O₅[M+H]⁺ m/z 441.13, found 441.17.

### Preparation of 2-(2-(5-(4-methoxybenzyl)-4-oxo-3-(trifluoromethyl)-4,5-dihydro-1H-pyrazolo[3,4-d]pyridazin-1-yl) propoxy)-N-methyl-N-(1-(5- (trifluoromethyl) pyrimidin-2-yl) piperidin-4-yl) acetamide (3806)

To a solution of 2-(2-(5-(4-methoxybenzyl)-4-oxo-3-(trifluoromethyl)-4,5-dihydro-1H-pyrazolo[3,4-d]pyridazin-1-yl)propoxy)acetic acid 3804 (80 mg, 0.18 mmol) in DCM (5 mL) were added N-methyl-1-(5-(trifluoromethyl)pyrimidin-2-yl)piperidin-4-amine hydrochloride 3805 (60 mg, 0.20 mmol), DIPEA (118 mg, 0.91 mmol), T3P (50% wt in EtOAc, 232 mg, 0.36 mmol) at rt successively. The reaction mixture was stirred at rt for 1 h. The reaction solution was quenched with water (10 mL) and extracted with DCM (10 mL×3). The combined organic phases were concentrated and purified by flash chromatography (eluting with PE/EtOAc=100:0 to 80:20) to afford 2-(2-(5-(4-methoxybenzyl)-4-oxo-3-(trifluoromethyl)-4,5-dihydro-1H-pyrazolo[3,4-d]pyridazin-1-yl)propoxy)-N-methyl-N-(1-(5-(trifluoromethyl)pyrimidin-2-yl)piperidin-4-yl)acetamide 3806 (70 mg, 90% purity, 50% yield) as a clear oil.

LCMS (ESI) calcd C₃₀H₃₂F₆N₈O₄[M+H]⁺ m/z 683.25, found 683.25.

### Preparation of N-methyl-2-(2-(4-oxo-3-(trifluoromethyl)-4,5-dihydro-1H-pyrazolo[3,4-d]pyridazin-1-yl) propoxy)-N-(1-(5-(trifluoromethyl) pyrimidin-2-yl) piperidin-4-yl) acetamide (mixture of 460 and 461)

To a stirred solution of 2-(2-(5-(4-methoxybenzyl)-4-oxo-3-(trifluoromethyl)-4,5-dihydro-1H-pyrazolo[3,4-d]pyridazin-1-yl)propoxy)-N-methyl-N-(1-(5-(trifluoromethyl)pyrimidin-2-yl)piperidin-4-yl) acetamide 3806 (90 mg, 0.13 mmol) in TFA (3 mL) was added TfOH (0.06 mL) at rt. The mixture was stirred at rt for 10 min. The solvent was adjusted to pH 7-8 with saturated NaHCO₃ at 0° C. and extracted with DCM. The combined organic phases were washed with water and brine, dried over sodium sulfate, concentrated under vacuum, and purified by C₁₈ column (Agela 40 g, mobile phase: ACN-H₂O (0.1% FA), gradient: 47-49) to afford N-methyl-2-(2-(4-oxo-3-(trifluoromethyl)-4,5-dihydro-1H-pyrazolo[3,4-d]pyridazin-1-yl)propoxy)-N-(1-(5-(trifluoromethyl) pyrimidin-2-yl) piperidin-4-yl) acetamide (mixture of compounds 460 and 461) (50 mg) as a white solid.

### Chiral resolution of N-methyl-2-(2-(4-oxo-3-(trifluoromethyl)-4,5-dihydro-1H-pyrazolo[3,4-d]pyridazin-1-yl) propoxy)-N-(1-(5-(trifluoromethyl) pyrimidin-2-yl) piperidin-4-yl) acetamide (compounds 460 and 461)

Compounds 460 and 461 were separated by SFC (Column: Daicel OJ-H 20 mm I.D.×250 mmL 5 m; Mobile phase: CO₂/MeOH=80/20) and concentrated under reduced pressure to afford the first fraction as 460 (17.2 mg, white solid, 99% purity, 100% ee) and the second fraction as 461 (17.2 mg, white solid, 99% purity, 100% ee).

### Compound 460

¹H NMR (400 MHz, DMSO-d₆, ppm) δ:12.99-12.75 (m, 1H), 8.80-8.58 (m, 3H), 5.39-5.15 (m, 1H), 4.88-4.68 (m, 2H), 4.54-4.41 (m, 0.6H), 4.26-4.17 (m, 1H), 4.11-4.06 (m, 1H), 3.87-3.61 (m, 2.4H), 3.04-2.94 (m, 1H), 2.94-2.78 (m, 1H), 2.58 (s, 1H), 1.69-1.41 (m, 7H).

LCMS (ESI) calcd. for C₂₂H₂₄F₆N₈O₃[M+H]⁺ m/z 563.19, found 563.25.

### Compound 461

¹H NMR (400 MHz, DMSO-d₆, ppm) δ:12.98-12.77 (m, 1H), 8.75-8.64 (m, 3H), 5.32-5.19 (m, 1H), 4.86-4.72 (m, 2H), 4.52-4.42 (m, 0.6H), 4.27-4.17 (m, 1H), 4.12-4.06 (m, 1H), 3.87-3.64 (m, 2.4H), 3.04-2.95 (m, 1H), 2.92-2.81 (m, 1H), 2.58 (s, 1H), 1.71-1.39 (m, 7H).

LCMS (ESI) calcd. for C₂₂H₂₄F₆N₈O₃[M+H]⁺ m/z 563.19, found 563.25.

### 30. Synthesis of 4-methyl-1-(2-(3-oxo-3-(4-(5-(trifluoromethyl)pyrimidin-2-yl)piperazin-1-yl)propoxy)ethyl)pyrido[2,3-d]pyridazine-2,5(1H,6H)-dione (compound 470)

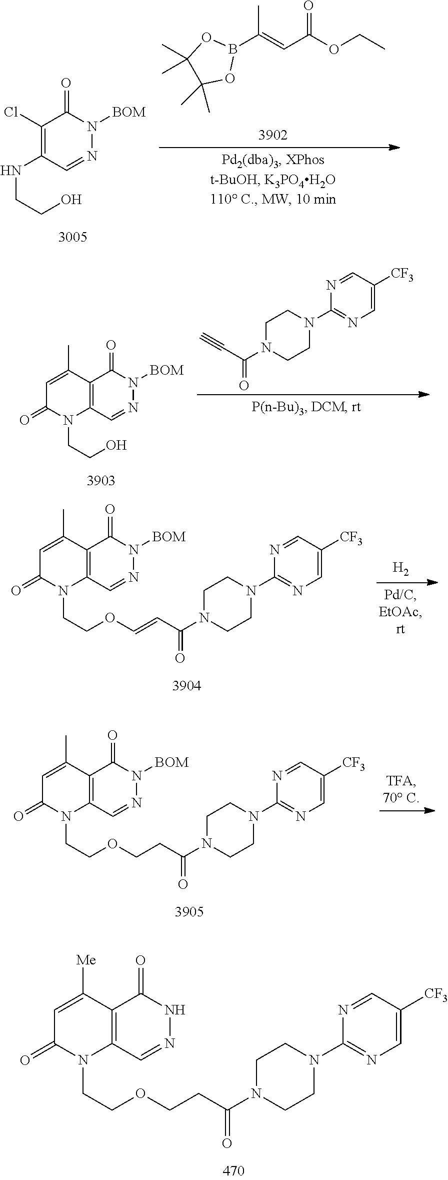

### Preparation of 6-((benzyloxy)methyl)-1-(2-hydroxyethyl)-4-methylpyrido[2,3-d]pyridazine-2,5(1H,6H)-dione (3903)

To a solution of 2-((benzyloxy)methyl)-4-chloro-5-((2-hydroxyethyl)amino)pyridazin-3(2H)-one 3005 (200 mg, 0.646 mmol) and ethyl (Z)-3-(4,4,5,5-tetramethyl-1,3,2-dioxaborolan-2-yl)but-2-enoate 3902 (186 mg, 0.775 mmol) in t-BuOH (10 mL) was added Pd₂(dba)₃ (59 mg, 0.0646 mmol), XPhos (154 mg, 0.323 mmol) and K₃PO₄H₂O (445 mg, 1.937 mmol) successively. The mixture was heated at 110° C. in a microwave reactor for 10 minutes under an atmosphere of N₂. The resulting solution was evaporated under reduced pressure to obtain a deep brown solid crude which was purified by flash silica chromatography (eluting with PE/EtOAc=50:50 to 0:100) to give 6-((benzyloxy)methyl)-1-(2-hydroxyethyl)-4-methylpyrido[2,3-d]pyridazine-2,5(1H,6H)-dione 3903 (50 mg, 90% purity, 23% yield) as a yellow solid.

LCMS (ESI) calcd for C₁₈H₁₉N₃O₄ [M+H]⁺ m/z 342.14, found 342.00.

### Preparation of (E)-6-((benzyloxy)methyl)-4-methyl-1-(2-((3-oxo-3-(4-(5-(trifluoromethyl)pyrimidin-2-yl)piperazin-1-yl)prop-1-en-1-yl)oxy)ethyl)pyrido[2,3-d]pyridazine-2,5(1H,6H)-dione (3904)

To a solution of 6-((benzyloxy)methyl)-1-(2-hydroxyethyl)-4-methylpyrido[2,3-d]pyridazine-2,5(1H,6H)-dione 3903 (50 mg, 0.613 mmol) and 1-(4-(5-(trifluoromethyl)pyrimidin-2-yl)piperazin-1-yl)prop-2-yn-1-one (40 mg, 0.1172 mmol) in dry DCM (5 mL) was added P(n-Bu)₃ (12 mg, 0.0586 mmol) at rt. The reaction mixture was stirred at rt for 2 h. The reaction solution was concentrated under reduced pressure. The residue was purified by silica gel column (eluting with PE/EtOAc=90:10 to 0:100) to obtain (E)-6-((benzyloxy)methyl)-4-methyl-1-(2-((3-oxo-3-(4-(5-(trifluoromethyl)pyrimidin-2-yl)piperazin-1-yl)prop-1-en-1-yl)oxy)ethyl)pyrido[2,3-d]pyridazine-2,5(1H,6H)-dione 3904 (60 mg, 90% purity, 49% yield) as white solid.

LCMS (ESI) calcd for C₃₀H₃₀F₃N₇O₅[M+H]⁺ m/z 626.23, found 626.30.

### Preparation of 6-((benzyloxy)methyl)-4-methyl-1-(2-(3-oxo-3-(4-(5-(trifluoromethyl)pyrimidin-2-yl)piperazin-1-yl)propoxy)ethyl)pyrido[2,3-d]pyridazine-2,5(1H,6H)-dione (3905)

To a solution of (E)-6-((benzyloxy)methyl)-4-methyl-1-(2-((3-oxo-3-(4-(5-(trifluoromethyl)pyrimidin-2-yl)piperazin-1-yl)prop-1-en-1-yl)oxy)ethyl)pyrido[2,3-d]pyridazine-2,5(1H,6H)-dione 3904 (60 mg, 0.26 mmol) and 10% Pd/C (6 mg) in EtOAc (15 mL) was stirred at room temperature for 18 h under H₂ atmosphere. The resulting solution was filtered through celite, and the filter cake was washed with DCM (5 mL×3). The filtrate was concentrated under reduced pressure to give 6-((benzyloxy)methyl)-4-methyl-1-(2-(3-oxo-3-(4-(5-(trifluoromethyl)pyrimidin-2-yl)piperazin-1-yl)propoxy)ethyl)pyrido[2,3-d]pyridazine-2,5(1H,6H)-dione 3905 (50 mg, 90% purity, 73% yield) as a yellow solid.

LCMS (ESI) calcd for C₃₀H₃₂F₃N₇O₅[M+H]⁺ m/z 628.24, found 628.15.

### Preparation of 4-methyl-1-(2-(3-oxo-3-(4-(5-(trifluoromethyl)pyrimidin-2-yl)piperazin-1-yl)propoxy)ethyl)pyrido[2,3-d]pyridazine-2,5(1H,6H)-dione (compound 470)

A solution of 6-((benzyloxy)methyl)-4-methyl-1-(2-(3-oxo-3-(4-(5-(trifluoromethyl)pyrimidin-2-yl)piperazin-1-yl)propoxy)ethyl)pyrido[2,3-d]pyridazine-2,5(1H,6H)-dione 3905 (50 mg, 0.085 mmol) in TFA (10 mL) was heated at 70° C. for 1 h. The solution was concentrated under reduced pressure to remove most TFA. The residue was diluted with DCM (50 mL) and then adjusted pH to 8 with saturated aqueous NaHCO₃ at 0° C. The basified solution was extracted with DCM (10 mL×3). The combined organic layer was washed with brine, dried over Na₂SO₄, and concentrated under reduced pressure. The crude product was purified by C₁₈ column (mobile phase: ACN-H₂O (0.1% FA), gradient: 25%-65%) to obtain 4-methyl-1-(2-(3-oxo-3-(4-(5-(trifluoromethyl)pyrimidin-2-yl)piperazin-1-yl)propoxy)ethyl)pyrido[2,3-d]pyridazine-2,5(1H,6H)-dione 470 (7.3 mg, 98% purity, 31% yield) as a white solid.

¹H NMR (400 MHz, DMSO-d₆, ppm) δ: 12.89 (s, 1H), 8.73 (s, 2H), 8.35 (s, 1H), 6.56 (s, 1H), 4.41 (t, J=5.2 Hz, 2H), 3.86-3.73 (m, 4H), 3.71-3.58 (m, 4H), 3.57-3.42 (m, 4H), 2.56 (s, 3H), 2.53-2.51 (m, 2H).

LCMS (ESI) calcd for C₂₂H₂₄F₃N₇O₄[M+H]⁺ m/z 508.18, found 508.24.

### 31. Synthesis of 1-(1-(3-oxo-3-(4-(5-(trifluoromethyl)pyrimidin-2-yl)piperazin-1-yl)propoxy)propan-2-yl-2-d)-3-(trifluoromethyl)-1,5-dihydro-4H-pyrrolo[2,3-d]pyridazin-4-one (compounds 476 and 477)

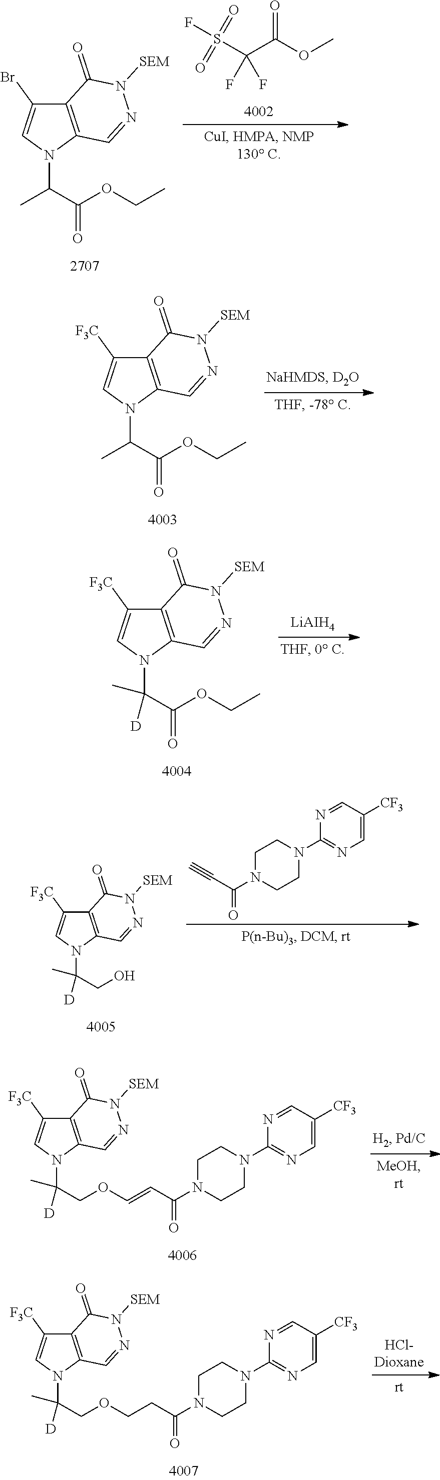

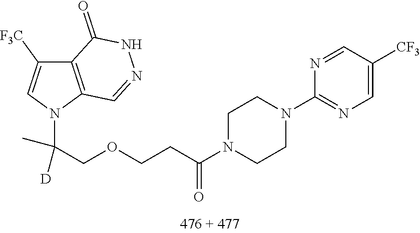 [SMILES: [2H]C(C)(COCCC(=O)N1CCN(c2ncc(C(F)(F)F)cn2)CC1)n1cc(C(F)(F)F)c2c(=O)[nH]ncc21]

### Preparation of ethyl 2-(4-oxo-3-(trifluoromethyl)-5-((2-(trimethylsilyl)ethoxy)methyl)-4,5-dihydro-1H-pyrrolo[2,3-d]pyridazin-1-yl)propanoate (4003)

To a solution of ethyl 2-(3-bromo-4-oxo-5-((2-(trimethylsilyl)ethoxy)methyl)-4,5-dihydro-1H-pyrrolo[2,3-d]pyridazin-1-yl)propanoate 2707 (1 g, 2.2 mmol) in NMP (20 mL) was added CuI (0.84 g, 4.4 mmol) and HMPA (1.97 g, 11.0 mmol) successively at rt. The reaction mixture was stirred at 130° C. under N₂ atmosphere. Methyl 2,2-difluoro-2-(fluorosulfonyl)acetate 4002 (2.1 g, 11.0 mmol) was added dropwise slowly at 130° C. The reaction mixture was stirred at 130° C. for 3 h. The reaction mixture was poured into water and then extracted with EtOAc (100 mL×3). The combined organic layers were washed with brine (100 mL×3), dried over Na₂SO₄ and concentrated under reduced pressure. The residue was purified by flash column chromatography (eluting with PE/EtOAc=85:15 to 60:40) to afford ethyl 2-(4-oxo-3-(trifluoromethyl)-5-((2-(trimethylsilyl)ethoxy)methyl)-4,5-dihydro-1H-pyrrolo[2,3-d]pyridazin-1-yl)propanoate 4003 (500 mg, 90% purity, 45% yield) as a yellow oil.

LCMS (ESI) calcd for C₁₈H₂₆F₃N₃O₄Si [M−27]⁺ m/z 406.16, found 406.20.

### Preparation of ethyl 2-(4-oxo-3-(trifluoromethyl)-5-((2-(trimethylsilyl)ethoxy)methyl)-4,5-dihydro-1H-pyrrolo[2,3-d]pyridazin-1-yl)propanoate-2-d (4004)

To a solution of ethyl 2-(4-oxo-3-(trifluoromethyl)-5-((2-(trimethylsilyl)ethoxy)methyl)-4,5-dihydro-1H-pyrrolo[2,3-d]pyridazin-1-yl)propanoate 4003 (500 mg, 1.15 mmol) in THE (15 mL) was added NaHMDS (1.15 mL, 2.3 mmol, 2 M in THF) at −78° C. under N₂ atmosphere. The reaction mixture was stirred at −78° C. for 2 h. The reaction mixture was quenched with D₂O (5 mL) at −78° C., the resulting solution was extracted with EtOAc (15 mL×3). The combined organic layers were concentrated under reduced pressure. The residue was purified by flash column chromatography (eluting with PE/EtOAc=85:15 to 50:50) to afford ethyl 2-(4-oxo-3-(trifluoromethyl)-5-((2-(trimethylsilyl)ethoxy)methyl)-4,5-dihydro-1H-pyrrolo[2,3-d]pyridazin-1-yl)propanoate-2-d 4004 (335 mg, 90% purity, 60% yield) as a colorless oil.

¹H NMR (400 MHz, DMSO-d₆, ppm) δ: 8.60 (s, 1H), 8.26 (s, 1H), 5.47 (s, 2H), 4.25-4.16 (m, 2H), 3.72-3.62 (m, 2H), 1.85 (s, 3H), 1.25-1.21 (m, 3H), 0.93-0.88 (m, 2H), −0.00 (s, 9H).

### Preparation of 1-(1-hydroxypropan-2-yl-2-d)-3-(trifluoromethyl)-5-((2-(trimethylsilyl)ethoxy)methyl)-1,5-dihydro-4H-pyrrolo[2,3-d]pyridazin-4-one (4005)

To a solution of ethyl 2-(4-oxo-3-(trifluoromethyl)-5-((2-(trimethylsilyl)ethoxy)methyl)-4,5-dihydro-1H-pyrrolo[2,3-d]pyridazin-1-yl)propanoate-2-d 4004 (120 mg, 0.28 mmol) in THE (10 mL) was added LiAlH₄ (17 mg, 0.41 mmol) at 0° C. The reaction mixture was stirred at 0° C. for 0.5 h. The reaction mixture was quenched with water, the aqueous layer was extracted with EtOAc (20 mL×3). The combined organic layers were concentrated under reduced pressure. The residue was purified by flash column chromatography (eluting with PE/EtOAc=85:15 to 20:80) to afford 1-(1-hydroxypropan-2-yl-2-d)-3-(trifluoromethyl)-5-((2-(trimethylsilyl)ethoxy)methyl)-1,5-dihydro-4H-pyrrolo[2,3-d]pyridazin-4-one 4005 (85 mg, 90% purity, 60% yield) as a white solid.

LCMS (ESI) calcd for C₁₆H₂₃DF₃N₃O₃Si [M+H]⁺ m/z 393.16, found 393.19.

### Preparation of (E)-1-(1-((3-oxo-3-(4-(5-(trifluoromethyl)pyrimidin-2-yl)piperazin-1-yl)prop-1-en-1-yl)oxy)propan-2-yl-2-d)-3-(trifluoromethyl)-5-((2-(trimethylsilyl)ethoxy)methyl)-1,5-dihydro-4H-pyrrolo[2,3-d]pyridazin-4-one (4006)

To a solution of 1-(1-hydroxypropan-2-yl-2-d)-3-(trifluoromethyl)-5-((2-(trimethylsilyl)ethoxy)methyl)-1,5-dihydro-4H-pyrrolo[2,3-d]pyridazin-4-one 4005 (85 mg, 0.22 mmol) in DCM (5 mL) were added 1-(4-(5-(trifluoromethyl)pyrimidin-2-yl)piperazin-1-yl)prop-2-yn-1-one (74 mg, 0.26 mmol) and P(n-Bu)₃ (22 mg, 0.11 mmol) at rt. The reaction mixture was stirred at room temperature for 2 h. The reaction mixture was diluted with DCM and water. The aqueous layer was extracted with DCM (10 mL×3). The combined organic layers were dried over Na₂SO₄ and concentrated under reduced pressure. The residue was purified by flash column chromatography (eluting with PE/EtOAc=20:80 to 0:100) to afford (E)-1-(1-((3-oxo-3-(4-(5-(trifluoromethyl)pyrimidin-2-yl)piperazin-1-yl)prop-1-en-1-yl)oxy)propan-2-yl-2-d)-3-(trifluoromethyl)-5-((2-(trimethylsilyl)ethoxy)methyl)-1,5-dihydro-4H-pyrrolo[2,3-d]pyridazin-4-one 4006 (135 mg, 90% purity, 83% yield) as a white solid.

LCMS (ESI) calcd for C₂₈H₃₄DF₆N₇O₄Si [M+H]⁺ m/z 677.25, found 677.15.

### Preparation of 1-(1-(3-oxo-3-(4-(5-(trifluoromethyl)pyrimidin-2-yl)piperazin-1-yl)propoxy)propan-2-yl-2-d)-3-(trifluoromethyl)-5-((2-(trimethylsilyl)ethoxy)methyl)-1,5-dihydro-4H-pyrrolo[2,3-d]pyridazin-4-one (4007)

A solution of (E)-1-(1-((3-oxo-3-(4-(5-(trifluoromethyl)pyrimidin-2-yl)piperazin-1-yl)prop-1-en-1-yl)oxy)propan-2-yl-2-d)-3-(trifluoromethyl)-5-((2-(trimethylsilyl)ethoxy)methyl)-1,5-dihydro-4H-pyrrolo[2,3-d]pyridazin-4-one 4006 (130 mg, 0.19 mmol) and Pd/C (15 mg) in MeOH (5 mL) was stirred at rt for 2 h under H₂ atmosphere. The resulting solution was filtered through diatomaceous earth and the filter cake was washed with DCM (5 mL×4). The filtrate was concentrated under reduced pressure to obtained 1-(1-(3-oxo-3-(4-(5-(trifluoromethyl)pyrimidin-2-yl)piperazin-1-yl)propoxy)propan-2-yl-2-d)-3-(trifluoromethyl)-5-((2-(trimethylsilyl)ethoxy)methyl)-1,5-dihydro-4H-pyrrolo[2,3-d]pyridazin-4-one 4007 (120 mg, 90% purity, 83% yield) as a white solid.

LCMS (ESI) calcd for C₂₈H₃₆DF₆N₇O₄Si [M+H]⁺ m/z 679.26, found 679.15.

### Preparation of 1-(1-(3-oxo-3-(4-(5-(trifluoromethyl)pyrimidin-2-yl)piperazin-1-yl)propoxy)propan-2-yl-2-d)-3-(trifluoromethyl)-1,5-dihydro-4H-pyrrolo[2,3-d]pyridazin-4-one (compounds 476 and 477)

A solution of 1-(1-(3-oxo-3-(4-(5-(trifluoromethyl)pyrimidin-2-yl)piperazin-1-yl)propoxy)propan-2-yl-2-d)-3-(trifluoromethyl)-5-((2-(trimethylsilyl)ethoxy)methyl)-1,5-dihydro-4H-pyrrolo[2,3-d]pyridazin-4-one 4007 (120 mg, 0.18 mmol) in HCl-Dioxane (10 mL, 4 M) was stirred at rt for 6 h. The reaction solution was concentrated under reduced pressure. The residue was purified by C18 column (mobile phase: ACN-H₂O (0.1% FA), gradient: 10-95) to give 1-(1-(3-oxo-3-(4-(5-(trifluoromethyl)pyrimidin-2-yl)piperazin-1-yl)propoxy)propan-2-yl-2-d)-3-(trifluoromethyl)-1,5-dihydro-4H-pyrrolo[2,3-d]pyridazin-4-one (mixture of 476 and 477) as a white solid.

### Chiral resolution of 1-(1-(3-oxo-3-(4-(5-(trifluoromethyl)pyrimidin-2-yl)piperazin-1-yl)propoxy)propan-2-yl-2-d)-3-(trifluoromethyl)-1,5-dihydro-4H-pyrrolo[2,3-d]pyridazin-4-one (compounds 476 and 477)

Compounds 476 and 477 were separated by SFC (Column: DAICEL OD-H 20 mm I.D.×250 mmL 5 m; Mobile phase: CO₂/IPA [0.1% NH₃ (7 M Solution in MeOH)]=75/25) and concentrated under reduced pressure to afford the first fraction as 476 (8.1 mg, 99% purity, 100% ee, white solid) and the second fraction as 477 (6.1 mg, 99% purity, 98% ee, white solid).

### Compound 476

¹H NMR (400 MHz, DMSO-d₆, ppm) δ: 12.55 (s, 1H), 8.73 (s, 2H), 8.49 (s, 1H), 8.13 (s, 1H), 3.82-3.70 (m, 6H), 3.69-3.55 (m, 2H), 3.53-3.40 (m, 4H), 2.49-2.46 (m, 2H), 1.46 (s, 3H).

LCMS (ESI) calcd for C₂₂H₂₂DF₆N₇O₃ [M+H]⁺ m/z 549.18 found 549.25.

### Compound 477

¹H NMR (400 MHz, DMSO-d₆, ppm) δ: 12.55 (s, 1H), 8.72 (s, 2H), 8.49 (s, 1H), 8.13 (s, 1H), 3.83-3.70 (m, 6H), 3.70-3.54 (m, 2H), 3.52-3.41 (m, 4H), 2.49-2.45 (m, 2H), 1.46 (s, 3 H).

LCMS (ESI) calcd for C₂₂H₂₂DF₆N₇O₃ [M+H]⁺ m/z 549.18 found 549.25.

### 32. Synthesis of 2-(2-(4-oxo-3-(trifluoromethyl)-4,5-dihydro-1H-pyrazolo[3,4-d]pyridazin-1-yl) propoxy)-N-(1-(5-(trifluoromethyl) pyrimidin-2-yl) piperidin-4-yl) acetamide (compounds 479 and 480)

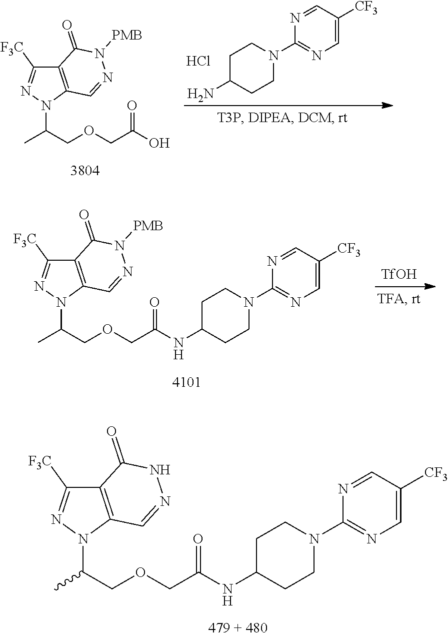

### Preparation of 2-(2-(5-(4-methoxybenzyl)-4-oxo-3-(trifluoromethyl)-4,5-dihydro-1H-pyrazolo[3,4-d]pyridazin-1-yl) propoxy)-N-(1-(5-(trifluoromethyl) pyrimidin-2-yl) piperidin-4-yl) a cetamide (4101)

To a solution of 2-(2-(5-(4-methoxybenzyl)-4-oxo-3-(trifluoromethyl)-4,5-dihydro-1H-pyrazolo[3,4-d]pyridazin-1-yl)propoxy)acetic acid 3804 (80 mg, 0.18 mmol) in DCM (4 mL) were added 1-(5-(trifluoromethyl)pyrimidin-2-yl)piperidin-4-amine hydrochloride (78 mg, 0.27 mmol), DIPEA (118 mg, 0.91 mmol), T3P (50% wt in EtOAc, 232 mg, 0.36 mmol) at rt successively. The reaction mixture was stirred at rt for 1 h. The reaction solution was quenched with water (10 mL) and extracted with DCM (15 mL×3). The combined organic phases were concentrated and purified by flash chromatography (eluting with DCM/MeOH=100:0 to 90:10) to afford 2-(2-(5-(4-methoxybenzyl)-4-oxo-3-(trifluoromethyl)-4,5-dihydro-1H-pyrazolo[3,4-d]pyridazin-1-yl) propoxy)-N-(1-(5-(trifluoromethyl) pyrimidin-2-yl) piperidin-4-yl) acetamide 4101 (70 mg, 90% purity, 51% yield) as a yellow oil.

LCMS (ESI) calcd C₂₉H₃₀F₆N₈O₄[M+H]+m/z 669.23, found 669.15.

### Preparation of 2-(2-(4-oxo-3-(trifluoromethyl)-4,5-dihydro-1H-pyrazolo[3,4-d]pyridazin-1-yl) propoxy)-N-(1-(5-(trifluoromethyl) pyrimidin-2-yl) piperidin-4-yl) acetamide (compound s 479 and 480)

To a stirred solution of 2-(2-(5-(4-methoxybenzyl)-4-oxo-3-(trifluoromethyl)-4,5-dihydro-1H-pyrazolo[3,4-d]pyridazin-1-yl)propoxy)-N-(1-(5-(trifluoromethyl) pyrimidin-2-yl) piperidin-4-yl) acetamide 4101 (65 mg, 0.10 mmol) in TFA (3 mL) was added TfOH (0.06 mL) at rt. The mixture was stirred at rt for 10 min. The solvent was adjusted to pH 7-8 with saturated NaHCO₃ at 0° C. and then extracted with DCM. The combined organic phases were washed with water and brine, dried over sodium sulfate, concentrated under vacuum, and purified by C₁₈ column (Agela 40 g, mobile phase: ACN-H₂O (0.1% FA), gradient: 45-47) to afford 2-(2-(4-oxo-3-(trifluoromethyl)-4,5-dihydro-1H-pyrazolo[3,4-d]pyridazin-1-yl) propoxy)-N-(1-(5-(trifluoromethyl) pyrimidin-2-yl) piperidin-4-yl) acetamide (mixture of 479 and 480) (20 mg, 98% purity, 36% yield) as a white solid.

### Chiral resolution of 2-(2-(4-oxo-3-(trifluoromethyl)-4,5-dihydro-1H-pyrazolo[3,4-d]pyridazin-1-yl) propoxy)-N-(1-(5-(trifluoromethyl) pyrimidin-2-yl) piperidin-4-yl) acetamide (compounds 479 and 480)

Compounds 479 and 480 were separated by SFC (Column: Daicel OJ-H 30 mm I.D.×250 mmL 10 m; Mobile phase: CO₂/MeOH=80/20) and concentrated under reduced pressure to afford the first fraction as 479 (13.1 mg, white solid, 97% purity, 99% ee) and the second fraction as 480 (9.8 mg, white solid, 98% purity, 100% ee).

### Compound 479

¹H NMR (400 MHz, DMSO-d₆, ppm) δ: 12.87 (s, 1H), 8.73 (s, 3H), 8.70 (s, 2H), 7.42 (d, J=8.0 Hz, 1H), 5.34-5.09 (m, 1H), 4.59 (d, J=13.2 Hz, 2H), 4.02-3.73 (m, 5H), 3.13 (t, J=11.8 Hz, 2H), 1.82-1.65 (m, 2H), 1.51 (d, J=6.4 Hz, 3H), 1.40-1.27 (m, 2H).

LCMS (ESI) calcd. for C₂₁H₂₂F₆N₈O₃[M+H]⁺ m/z 549.17, found 549.24.

### Compound 480

¹H NMR (400 MHz, DMSO-d₆, ppm) δ: 12.87 (s, 1H), 8.73 (s, 1H), 8.70 (s, 2H), 7.42 (d, J=8.0 Hz, 1H), 5.33-5.16 (m, 1H), 4.59 (d, J=13.2 Hz, 2H), 3.95-3.67 (m, 5H), 3.13 (t, J=11.8 Hz, 2H), 1.78-1.63 (m, 2H), 1.51 (d, J=6.8 Hz, 3H), 1.39-1.27 (m, 2H).

LCMS (ESI) calcd. for C₂₁H₂₂F₆N₈O₃[M+H]⁺ m/z 549.17, found 549.29.

### Assays

Exemplary compounds of the invention were prepared and tested to determine their effect as PARP7 inhibitors. A typical assay is described below.

### PARP7 Biochemical Dissociation-Enhanced Lanthanide Fluorescence Immunoassay (DELFIA Assay)

Optiplate HB 384-well plates were coated with anti-FLAG antibody, supplied as a 4 mg/ml solution, using a Na₂CO₃/HCO₃ coating buffer at pH 9.6, overnight at 4° C., in order to achieve a final immobilisation per well of 0.3 μg. Wells were then washed 3× in coating wash buffer (PBS/0.05% Tween (v/v)), blocked with 2% BSA (w/v) in coating wash buffer and washed 3 further times prior to assay. For the assay 20 μl of 12.5-37.5 nM recombinant human Flag-tagged PARP7 (amino acids 456-657) was added to each well of the 384-well plate for 30 min at room temperature. 50 nl of test compound in DMSO was added using pintool technology and plates were incubated for a further 30 min at room temperature. 5 μl of 15 μM biotin-NAD⁺ assay solution in 20 mM HEPES (pH 7.5), 100 mM NaCl, 2 mM DTT, 0.1% BSA (w/v), 0.02% Tween (v/v) assay buffer was then added and MARylation proceeded for 2-3 h at room temperature prior to the addition of 5 μl of 12 mM NAD⁺ quenching solution. After 30 min at room temperature, assay solution was removed and following washing 5 times, 100 μl of a 1:1000 dilution of DELFIA Eu-N1 Streptavidin reagent was added. Plates were then incubated for 30 min at room temperature. Reaction mixture was removed and plates washed 5 times prior to the addition of 25 μl DELFIA enhancement solution. Following incubation for 30 min at room temperature, fluorescence was read on either an Envision or Pherastar FS (Ex337 nm, Em620 nm).

Typically compounds were tested from 10-20 μM at 0.5 log intervals in 10-12-point concentration-response curves to determine IC₅₀ values. Data was analysed using ActivityBase software and replicate values for the low (without enzyme, 0.2% DMSO) and high (0.2% DMSO) % controls were averaged and the data obtained from the test compounds expressed as a % of 100% using the below formulae:

% activity=100*(value−low control)/(high control−low control)
% activity data was fitted with 4-parameter non-linear regression equation to obtain IC50 values.

The IC50 values for a variety of test compounds are shown in Table 1.

TABLE 1
Results of Parp 7 assay for selected compounds

| | PARP7 |
|---|---|
| Compound | activity |
| 1 | +++ |
| 2 | + |
| 3 | ++ |
| 4 | +++ |
| 5 | + |
| 6 | ++ |
| 7 | ++ |
| 8 | +++ |
| 9 | ++ |
| 10 | +++ |
| 11 | + |
| 12 | ++ |
| 13 | +++ |
| 14 | + |
| 15 | ++ |
| 16 | ++ |
| 17 | + |
| 18 | ++ |
| 19 | + |
| 20 | ++ |
| 21 | + |
| 22 | + |
| 23 | ++ |
| 24 | ++ |
| 25 | +++ |
| 26 | + |
| 27 | + |
| 28 | ++ |
| 29 | ++ |
| 30 | +++ |
| 31 | +++ |
| 32 | + |
| 33 | ++ |
| 34 | + |
| 35 | ++ |
| 36 | + |
| 37 | + |
| 38 | + |
| 39 | + |
| 40 | ++ |
| 41 | + |
| 42 | +++ |
| 43 | ++ |
| 44 | +++ |
| 45 | +++ |
| 46 | ++ |
| 47 | + |
| 48 | +++ |
| 49 | ++ |
| 50 | +++ |
| 51 | ++ |
| 52 | ++ |
| 53 | ++ |
| 54 | ++ |
| 55 | +++ |
| 56 | + |
| 57 | +++ |
| 58 | ++ |
| 59 | ++ |
| 60 | +++ |
| 61 | ++ |
| 62 | ++ |
| 63 | +++ |
| 64 | +++ |
| 65 | +++ |
| 66 | ++ |
| 67 | +++ |
| 68 | ++ |
| 69 | ++ |
| 70 | + |
| 71 | +++ |
| 72 | + |
| 73 | +++ |
| 74 | +++ |
| 75 | +++ |
| 76 | ++ |
| 77 | +++ |
| 78 | ++ |
| 79 | +++ |
| 80 | +++ |
| 81 | +++ |
| 82 | +++ |
| 83 | ++ |
| 84 | ++ |
| 85 | +++ |
| 86 | +++ |
| 87 | +++ |
| 88 | +++ |
| 89 | +++ |
| 90 | +++ |
| 91 | +++ |
| 92 | ++ |
| 93 | + |
| 94 | + |
| 95 | ++ |
| 96 | +++ |
| 97 | +++ |
| 98 | +++ |
| 99 | +++ |
| 100 | +++ |
| 101 | +++ |
| 102 | +++ |
| 103 | ++ |
| 104 | ++ |
| 105 | +++ |
| 106 | +++ |
| 107 | +++ |
| 108 | +++ |
| 109 | + |
| 110 | ++ |
| 111 | ++ |
| 112 | ++ |
| 113 | ++ |
| 114 | +++ |
| 115 | ++ |
| 116 | + |
| 117 | + |
| 118 | ++ |
| 119 | + |
| 120 | +++ |
| 121 | ++ |
| 122 | +++ |
| 123 | +++ |
| 124 | +++ |
| 125 | +++ |
| 126 | ++ |
| 127 | +++ |
| 128 | ++ |
| 129 | +++ |
| 130 | +++ |
| 131 | +++ |
| 132 | +++ |
| 133 | +++ |
| 134 | +++ |
| 135 | ++ |
| 136 | ++ |
| 137 | +++ |
| 138 | +++ |
| 139 | +++ |
| 140 | +++ |
| 141 | +++ |
| 142 | +++ |
| 143 | +++ |
| 144 | +++ |
| 145 | + |
| 146 | +++ |
| 147 | +++ |
| 148 | +++ |
| 149 | +++ |
| 150 | +++ |
| 151 | +++ |
| 152 | +++ |
| 153 | +++ |
| 154 | +++ |
| 155 | +++ |
| 156 | ++ |
| 157 | +++ |
| 158 | +++ |
| 159 | ++ |
| 160 | +++ |
| 161 | +++ |
| 162 | ++ |
| 163 | +++ |
| 164 | +++ |
| 165 | +++ |
| 166 | +++ |
| 167 | ++ |
| 168 | +++ |
| 169 | ++ |
| 170 | ++ |
| 171 | +++ |
| 172 | +++ |
| 173 | +++ |
| 174 | +++ |
| 175 | +++ |
| 176 | +++ |
| 177 | +++ |
| 178 | +++ |
| 179 | +++ |
| 180 | +++ |
| 181 | +++ |
| 182 | +++ |
| 183 | +++ |
| 184 | ++ |
| 185 | +++ |
| 186 | +++ |
| 187 | +++ |
| 188 | +++ |
| 189 | +++ |
| 190 | ++ |
| 191 | +++ |
| 192 | +++ |
| 193 | +++ |
| 194 | +++ |
| 195 | +++ |
| 196 | +++ |
| 197 | ++ |
| 198 | +++ |
| 199 | +++ |
| 200 | +++ |
| 201 | +++ |
| 202 | +++ |
| 203 | +++ |
| 204 | ++ |
| 205 | +++ |
| 206 | +++ |
| 207 | ++ |
| 208 | +++ |
| 209 | +++ |
| 210 | +++ |
| 211 | +++ |
| 212 | +++ |
| 213 | +++ |
| 214 | +++ |
| 215 | +++ |
| 216 | +++ |
| 217 | +++ |
| 218 | +++ |
| 219 | ++ |
| 220 | +++ |
| 221 | ++ |
| 222 | ++ |
| 223 | +++ |
| 224 | +++ |
| 225 | +++ |
| 226 | +++ |
| 227 | +++ |
| 228 | +++ |
| 229 | +++ |
| 230 | +++ |
| 231 | +++ |
| 232 | +++ |
| 233 | +++ |
| 234 | +++ |
| 235 | +++ |
| 236 | +++ |
| 237 | +++ |
| 238 | +++ |
| 239 | +++ |
| 240 | +++ |
| 241 | +++ |
| 242 | +++ |
| 243 | +++ |
| 244 | +++ |
| 245 | +++ |
| 246 | +++ |
| 247 | +++ |
| 248 | +++ |
| 249 | +++ |
| 250 | + |
| 251 | +++ |
| 252 | +++ |
| 253 | +++ |
| 254 | +++ |
| 255 | +++ |
| 256 | +++ |
| 257 | +++ |
| 258 | +++ |
| 259 | +++ |
| 260 | + |
| 261 | +++ |
| 262 | +++ |
| 263 | +++ |
| 264 | +++ |
| 265 | +++ |
| 266 | +++ |
| 267 | +++ |
| 268 | +++ |
| 269 | +++ |
| 270 | +++ |
| 271 | +++ |
| 272 | + |
| 273 | +++ |
| 274 | +++ |
| 275 | +++ |
| 276 | +++ |
| 277 | +++ |
| 278 | +++ |
| 279 | +++ |
| 280 | +++ |
| 281 | +++ |
| 282 | + |
| 283 | +++ |
| 284 | +++ |
| 285 | +++ |
| 286 | +++ |
| 287 | +++ |
| 288 | +++ |
| 289 | +++ |
| 290 | +++ |
| 291 | ++ |
| 292 | +++ |
| 293 | +++ |
| 294 | +++ |
| 295 | +++ |
| 296 | +++ |
| 297 | +++ |
| 298 | +++ |
| 299 | +++ |
| 300 | +++ |
| 301 | +++ |
| 302 | +++ |
| 303 | +++ |
| 304 | ++ |
| 305 | +++ |
| 306 | +++ |
| 307 | +++ |
| 308 | +++ |
| 309 | +++ |
| 310 | +++ |
| 311 | ++ |
| 312 | +++ |
| 313 | +++ |
| 314 | +++ |
| 315 | +++ |
| 316 | +++ |
| 317 | +++ |
| 318 | ++ |
| 319 | +++ |
| 320 | +++ |
| 321 | +++ |
| 322 | +++ |
| 323 | +++ |
| 324 | +++ |
| 325 | +++ |
| 326 | +++ |
| 327 | +++ |
| 328 | +++ |
| 329 | +++ |
| 330 | +++ |
| 331 | +++ |
| 332 | +++ |
| 333 | +++ |
| 334 | +++ |
| 335 | +++ |
| 336 | +++ |
| 337 | + |
| 338 | +++ |
| 339 | +++ |
| 340 | +++ |
| 341 | +++ |
| 342 | +++ |
| 343 | +++ |
| 344 | +++ |
| 345 | ++ |
| 346 | +++ |
| 347 | +++ |
| 348 | +++ |
| 349 | +++ |
| 350 | +++ |
| 351 | +++ |
| 352 | +++ |
| 353 | +++ |
| 354 | ++ |
| 355 | +++ |
| 356 | ++ |
| 357 | +++ |
| 358 | +++ |
| 359 | +++ |
| 360 | +++ |
| 361 | +++ |
| 362 | +++ |
| 363 | +++ |
| 364 | +++ |
| 365 | +++ |
| 366 | +++ |
| 367 | +++ |
| 368 | ++ |
| 369 | +++ |
| 370 | +++ |
| 371 | +++ |
| 372 | +++ |
| 373 | +++ |
| 374 | +++ |
| 375 | +++ |
| 376 | +++ |
| 377 | +++ |
| 378 | +++ |
| 379 | +++ |
| 380 | +++ |
| 381 | +++ |
| 382 | +++ |
| 383 | +++ |
| 384 | +++ |
| 385 | +++ |
| 386 | +++ |
| 387 | +++ |
| 388 | +++ |
| 389 | +++ |
| 390 | +++ |
| 391 | +++ |
| 392 | ++ |
| 393 | +++ |
| 394 | +++ |
| 395 | +++ |
| 396 | +++ |
| 397 | +++ |
| 398 | +++ |
| 399 | ++ |
| 400 | +++ |
| 401 | +++ |
| 402 | +++ |
| 403 | +++ |
| 404 | +++ |
| 405 | ++ |
| 406 | +++ |
| 407 | +++ |
| 408 | +++ |
| 409 | +++ |
| 410 | +++ |
| 411 | +++ |
| 412 | +++ |
| 413 | +++ |
| 414 | ++ |
| 415 | +++ |
| 416 | +++ |
| 417 | +++ |
| 418 | +++ |
| 419 | +++ |
| 420 | +++ |
| 421 | +++ |
| 422 | +++ |
| 423 | +++ |
| 424 | +++ |
| 425 | ++ |
| 426 | + |
| 427 | ++ |
| 428 | +++ |
| 429 | +++ |
| 430 | ++ |
| 431 | +++ |
| 432 | +++ |
| 433 | + |
| 434 | +++ |
| 435 | +++ |
| 436 | +++ |
| 437 | +++ |
| 438 | +++ |
| 439 | +++ |
| 440 | +++ |
| 441 | +++ |
| 442 | + |
| 443 | + |
| 444 | +++ |
| 445 | +++ |
| 446 | +++ |
| 447 | +++ |
| 448 | +++ |
| 449 | +++ |
| 450 | +++ |
| 451 | +++ |
| 452 | +++ |
| 453 | +++ |
| 454 | +++ |
| 455 | +++ |
| 456 | +++ |
| 457 | +++ |
| 458 | +++ |
| 459 | +++ |
| 460 | +++ |
| 461 | +++ |
| 462 | +++ |
| 463 | +++ |
| 464 | +++ |
| 465 | +++ |
| 466 | +++ |
| 467 | +++ |
| 468 | +++ |
| 469 | +++ |
| 470 | +++ |
| 471 | +++ |
| 472 | +++ |
| 473 | ++ |
| 474 | +++ |
| 475 | +++ |
| 476 | +++ |
| 477 | +++ |
| 478 | +++ |
| 479 | +++ |
| 480 | +++ |
| 481 | +++ |
| 482 | +++ |
| 483 | +++ |
| 484 | +++ |
| 485 | +++ |
| 486 | +++ |
| 487 | +++ |
| 488 | +++ |
| 489 | +++ |
| 490 | +++ |
| 491 | +++ |
| 492 | +++ |
| 493 | +++ |
| 494 | +++ |
| 495 | ++ |
| 496 | +++ |
| 497 | +++ |
| 498 | +++ |
| 499 | +++ |
| 500 | +++ |
| 501 | +++ |
| 502 | +++ |

Key:
+++ indicates IC₅₀ ≤100 nM
++ indicates IC₅₀ >100 nM and ≤1 μM
+ indicates IC₅₀ >1 μM and ≤10 μM

### Induction of Type I Interferon (Interferon β, IFN β) Production in Cancer Cells as a Cell-Based Measure of PARP7 Inhibition

Selected compounds were also tested for their ability to induce type I interferon (interferon β, IFNβ) production in cancer cells as a cell-based measure of PARP7 inhibition and its effects on stimulating the STING pathway signalling. FIG. 1 demonstrates the induction of IFNβ in the supernatants of murine CT26 and MC38 colorectal cancer cells following 24 hour incubation with compound 177 alone or in combination with a variety of chemotherapeutic agents. As can be seen, the combination of PARP7 inhibitor and chemotherapeutic agents augments activation of STING signalling as measured by IFNβ induction. Such data highlight the therapeutic utility of combining PARP7 inhibitors with chemotherapy or other agents that can induce aberrant cytosolic nucleic acids in order to further enhance an anti-tumour immune response.

A particularly suitable assay to measure IFNβ from mouse cancer cell supernatants is described below.

### High Sensitivity Mouse IFNβ ELISA Assay

CT26 and MC38 cells were seeded in 96-well microplates each at a density of 30,000 cells per well. Following overnight incubation, cells were treated with either DMSO, 10 μM compound 177, or various chemotherapeutic agents (final DMSO concentration of 0.2% v/v). Cells were also treated with 10 μM compound 177 in combination with various chemotherapeutic agents. After 24 hours, IFNβ was measured from cell supernatants using a sandwich enzyme linked immunosorbent assay (ELISA) assay kit (PBL Assay Sciences, catalogue number 42410-2) according to manufacturer's instructions.

1. A PARP7 inhibitor compound, which compound comprises the following formula: 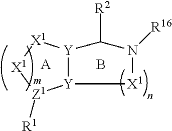
wherein each X¹ may be the same or different and is independently selected from C, N, O and S; each Y may be the same or different and is independently selected from C and N; Z¹ is independently selected from C and N; each X¹ may independently be unsubstituted, or may independently be substituted with H or a substituted or unsubstituted organic group; each Y may independently be unsubstituted, or may independently be substituted with H or a substituted or unsubstituted organic group; Z¹ may independently be further substituted with H or a substituted or unsubstituted organic group; m may be 1, 2, 3 or 4; n may be 1, 2, or 3; the bonds between all of the atoms in ring A may independently be single bonds or double bonds provided that when X¹ is O or S the bonds to that X¹ are single bonds; the bonds between all of the atoms in ring B may independently be single bonds or double bonds provided that when X¹ is O or S the bonds to that X¹ are single bonds;
and wherein R¹ may be attached to Z¹ by a single bond or a double bond and is a substituent of formula: 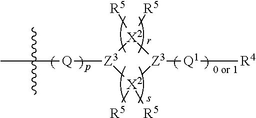
wherein each Q may be the same or different and is independently selected from C, N, O and S; each Q may independently be attached to another Q, or to Z³, by a single bond or a double bond; each Q may independently be unsubstituted, or may independently be substituted by H or a substituted or unsubstituted organic group; two or more Q atoms may form a ring together with their substituents; p is a number from 2 to 8; each Z³ may be the same or different and is independently selected from C and N; each X² may be the same or different and is independently selected from C, N, O and S; r is a number from 1 to 5; s is independently a number from 1 to 5; wherein Q¹ is selected from C, N, O and S and may be attached to Z³ and R⁴, by a single bond or a double bond and may be unsubstituted, or substituted by H or an organic group; and R⁴ is a substituted or unsubstituted organic group comprising a substituted or unsubstituted carbocyclic or heterocyclic ring; each bond in the ring comprised of Z³ and X² atoms may independently be a double bond or a single bond, provided that when X² is O or S the bonds to that X² are single bonds; each R⁵ may be present or absent depending on the number of bonds to, and the valence of, the X² atom attached to that R⁵; and wherein each R⁵ is independently selected from H or a substituted or unsubstituted organic group;
and wherein R² may be attached to ring B by a single bond or a double bond and is a substituted or unsubstituted organic group;
and wherein R¹⁶ may be present or absent and when present is selected from H, a C₁-C₆ alkyl group or a linear or branched C₁-C₆ halogenated alkyl group.

2. A compound according to claim 1, wherein R¹ has any of the following structures:

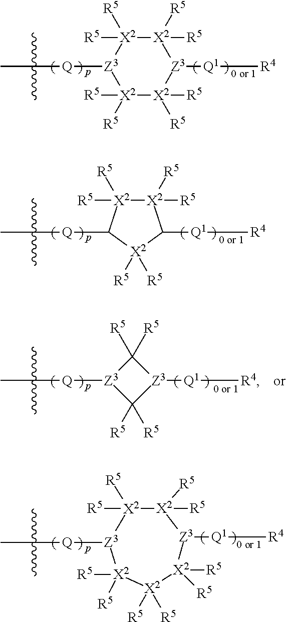

3. A compound according to claim 1, wherein R¹ has any of the following structures: 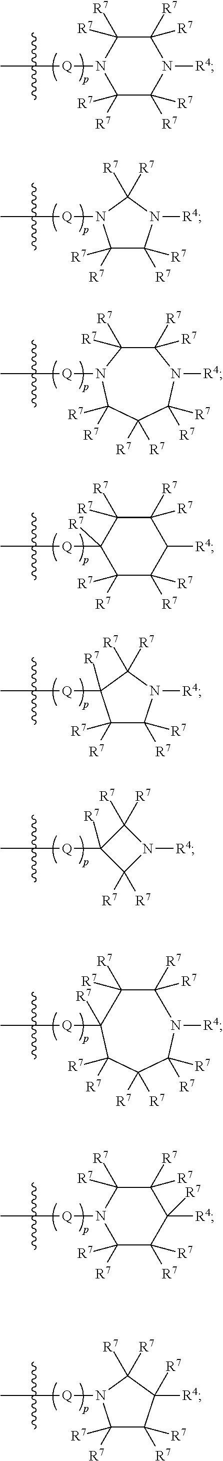 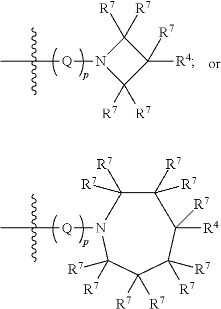
wherein R⁷ is selected from H or a substituted or unsubstituted organic group.

4. A compound according to claim 1, wherein the linking group -(Q)ₚ- has any of the following structures: 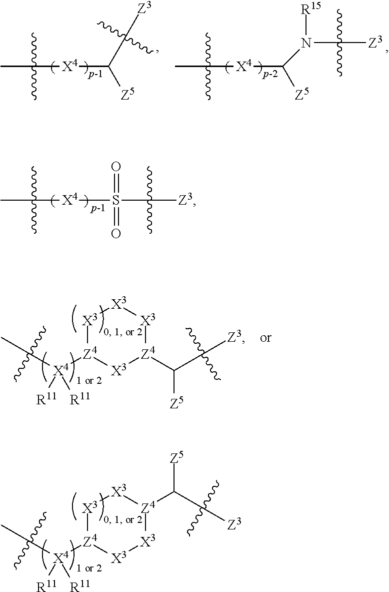
wherein each X³ may be the same or different and is independently selected from C, N, O and S; when C or N, each X³ may independently be unsubstituted or substituted with H or a substituted or unsubstituted organic group; each X⁴ may be the same or different and is independently selected from C, N, O and S; each Z⁴ may be the same or different and is independently selected from C and N; the bonds between all of the atoms of any ring may independently be single bonds or double bonds provided that when X³ is O or S the bonds to that X³ are single bonds; R¹¹ may be present or absent depending on the number of bonds and the valence of the X⁴ atom comprising that R¹¹; and wherein each R¹¹ is independently selected from H or a substituted or unsubstituted organic group; and wherein R¹⁵ is selected from H, a linear or branched C₁-C₆ alkyl group or a linear or branched C₁-C₆ halogenated alkyl group; and wherein Z⁵ may be attached via a single bond or a double bond and is selected from the following: 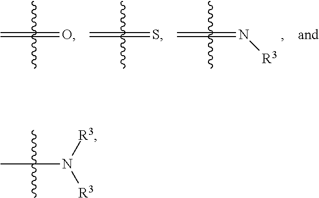
and
wherein each R³ may be the same or different and is independently selected from H and a substituted or unsubstituted organic group.

5. A compound according to claim 1, wherein the linking group -(Q)ₚ- is selected from the following: 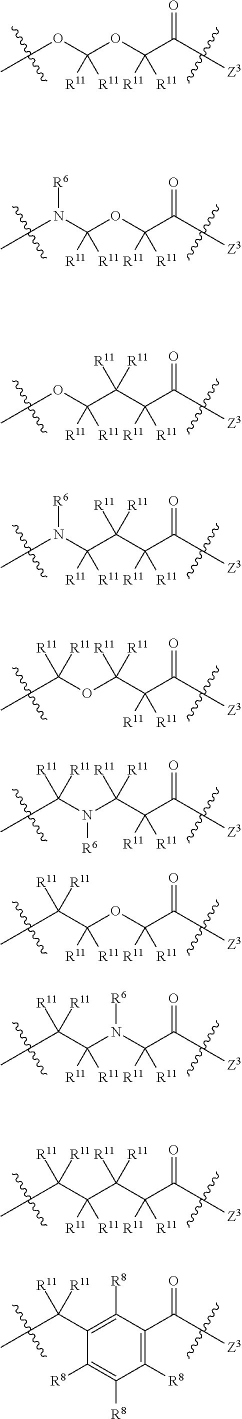 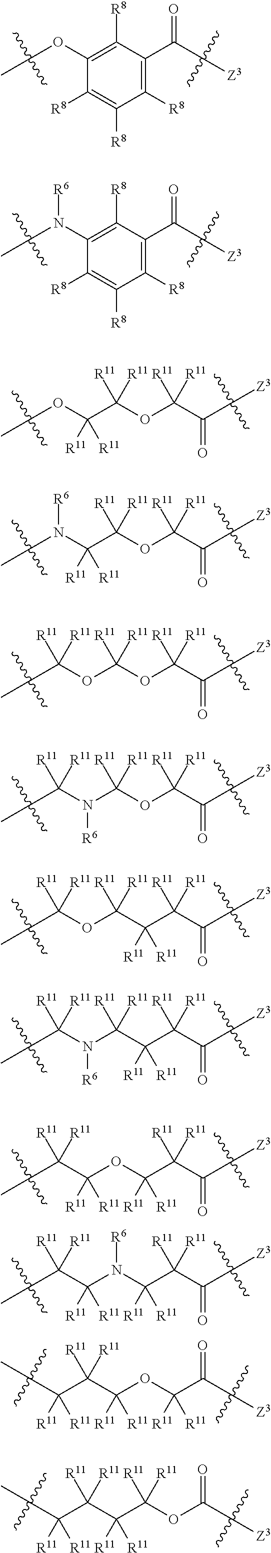 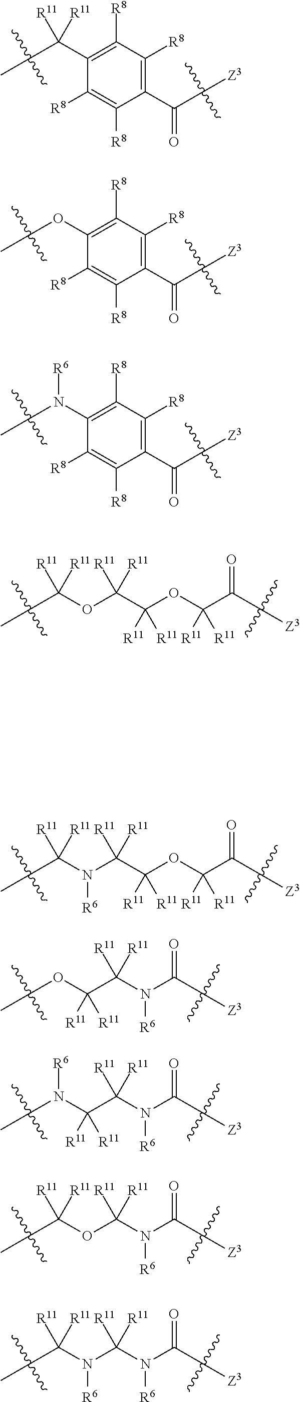 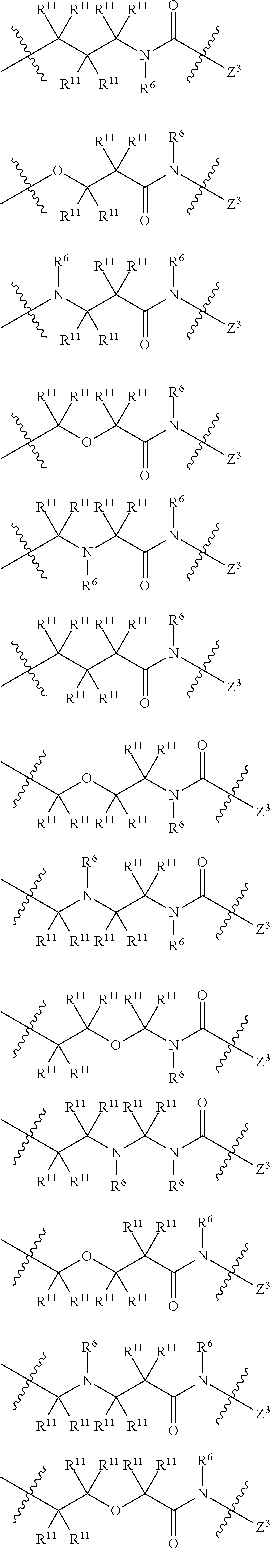 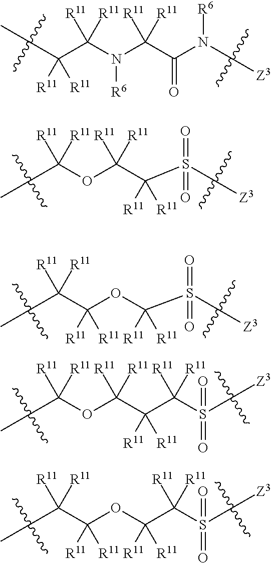
wherein each R⁶, R⁸, and R¹¹ is independently selected from H and a substituted or unsubstituted organic group.

6. A compound according to claim 1, wherein R⁴ may be attached via a single bond or a double bond and is selected from the following: 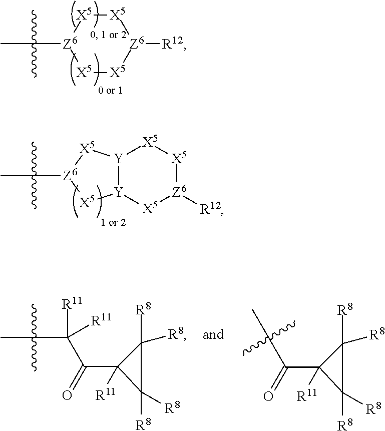
wherein each X⁵ may be the same or different and is independently selected from C, N, O and S; when C or N, each X⁵ may independently be unsubstituted or substituted with H or a substituted or unsubstituted organic group; each Z⁶ may be the same or different and is independently selected from C and N; the bonds between all of the atoms of any ring may independently be single bonds or double bonds provided that when X⁵ is O or S the bonds to that X⁵ are single bonds; R¹² may be present or absent depending on the number of bonds and the valence of the Z⁶ atom comprising that R¹²; wherein R¹² is independently selected from H or a substituted or unsubstituted organic group; wherein each R⁸ and each R¹¹ is independently selected from H or a substitute or unsubstituted organic group.

7. A compound according to claim 1, wherein R⁴ is selected from the following: 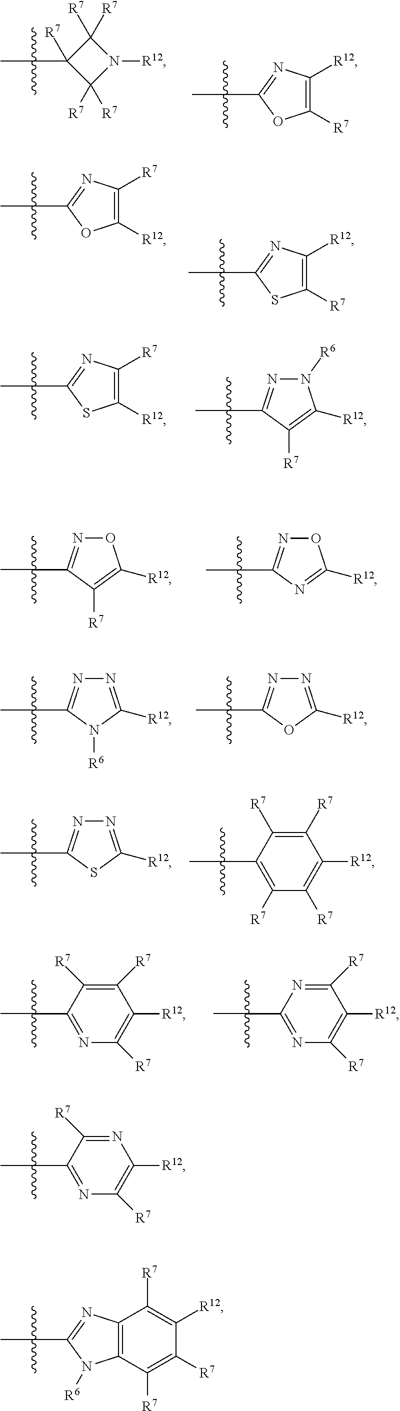 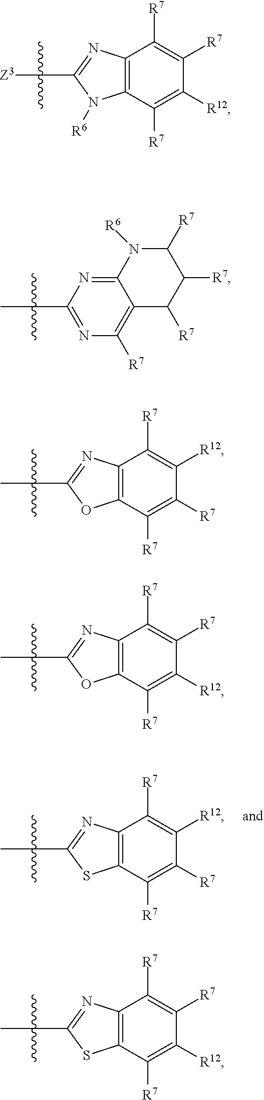
wherein R⁶, R⁷ and R¹² are each independently or a substituted or unsubstituted organic group.

8. A compound according to claim 1, wherein R² may be attached via a single bond or a double bond and is selected from the following: 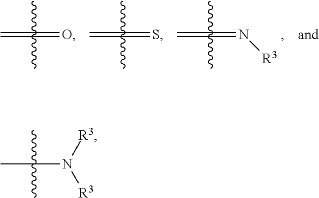
wherein each R³ may be the same or different and is independently selected from H and a substituted or unsubstituted organic group.

9. A compound according to claim 1, wherein R¹ is-selected from the following: 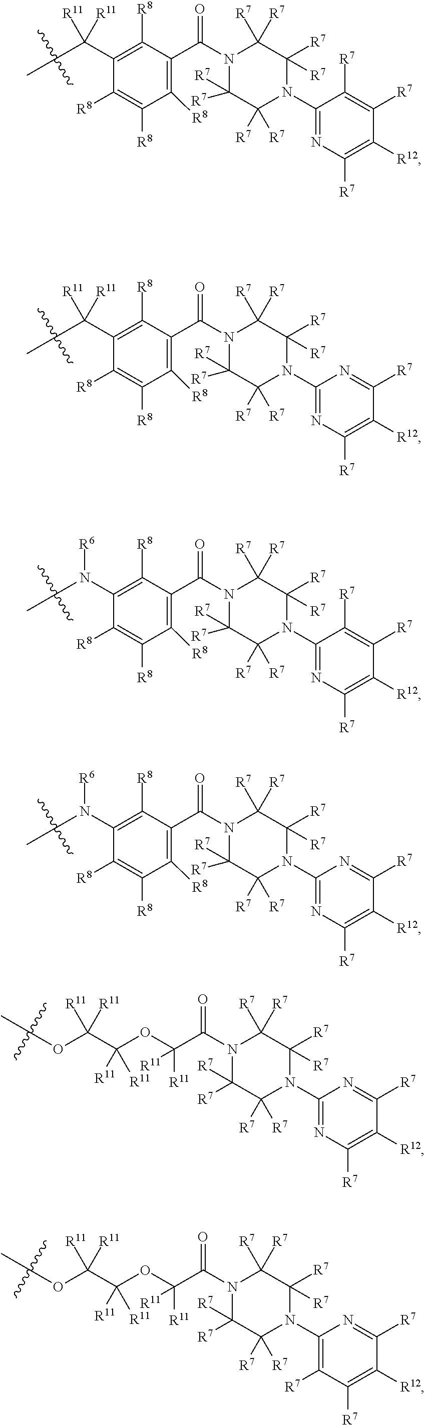 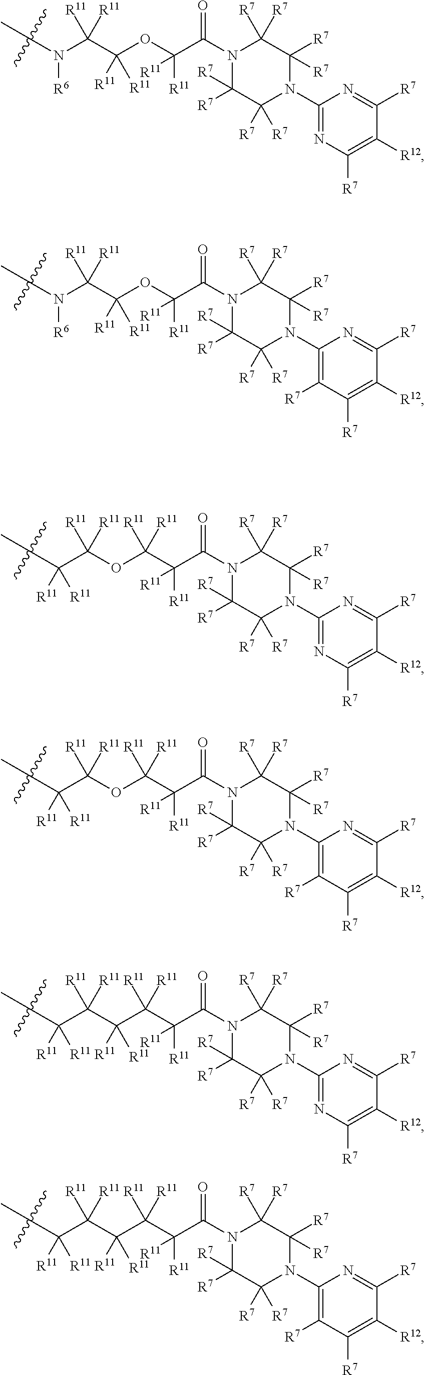 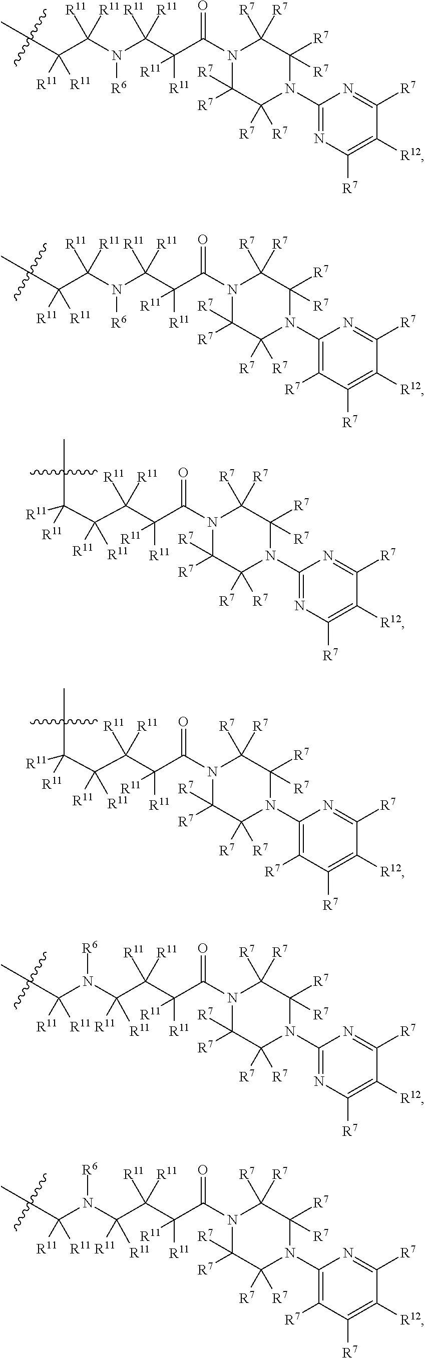 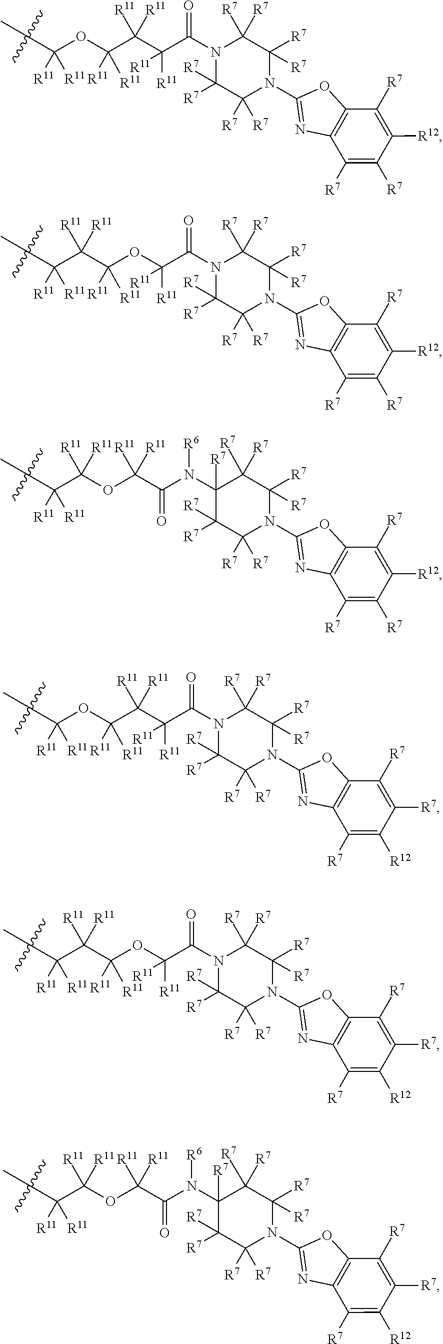 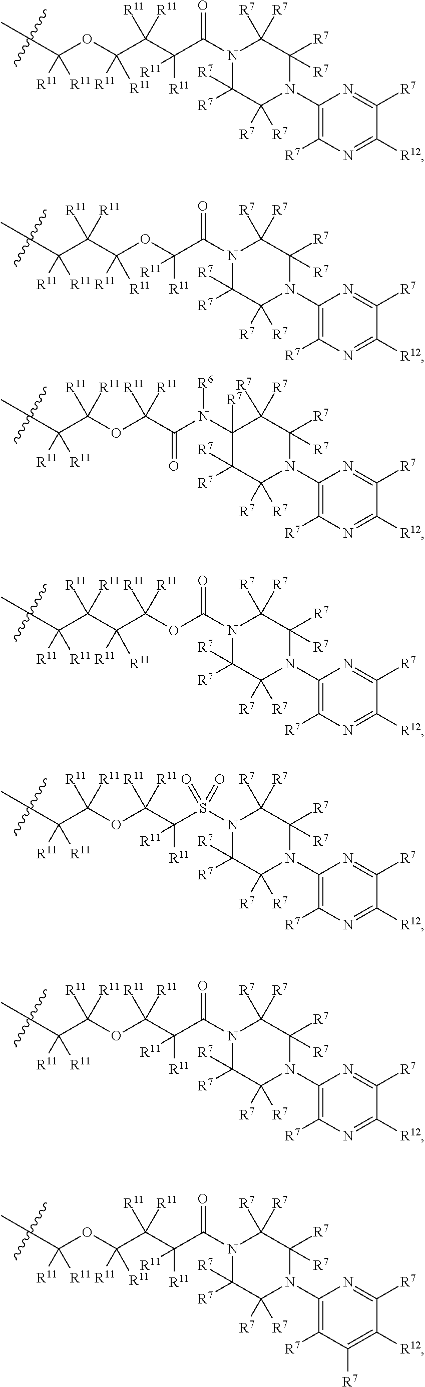 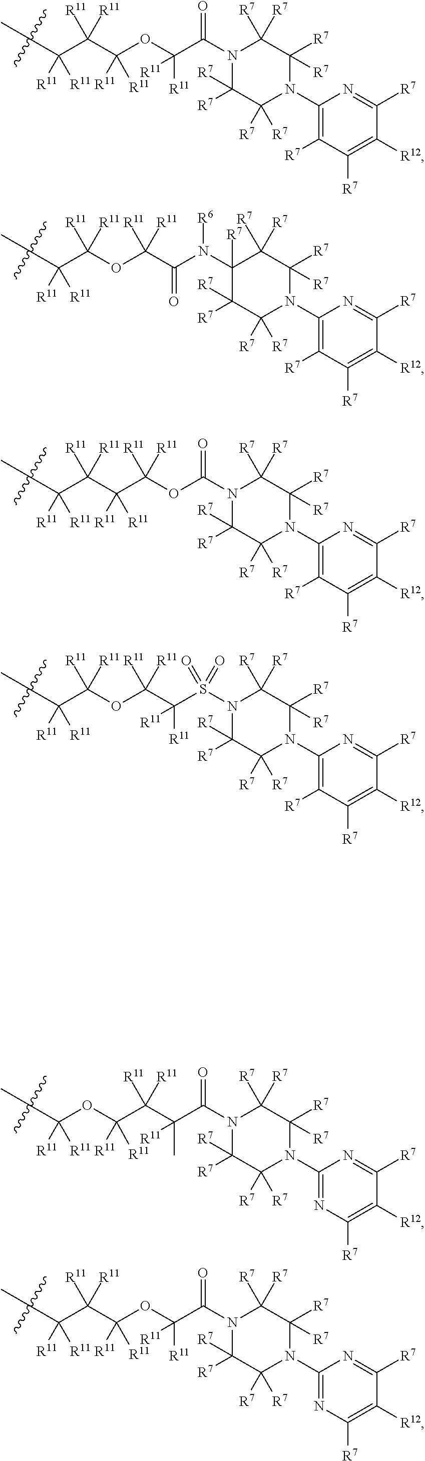 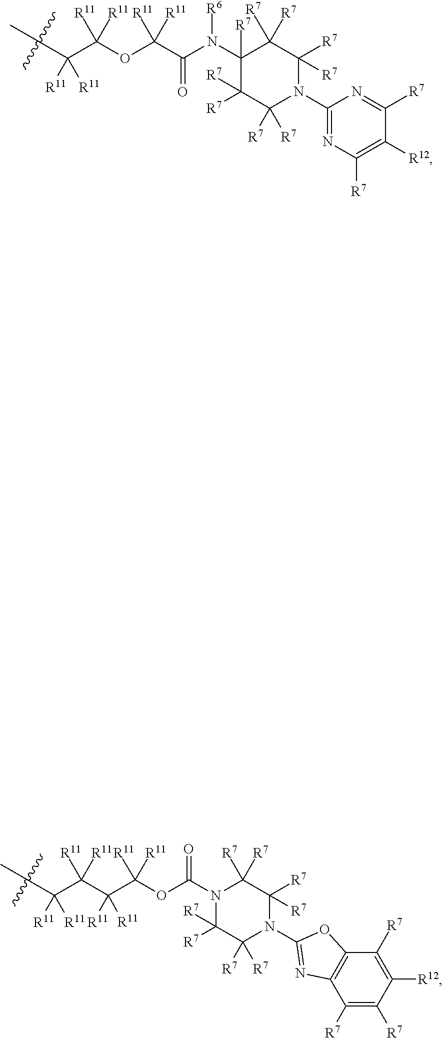 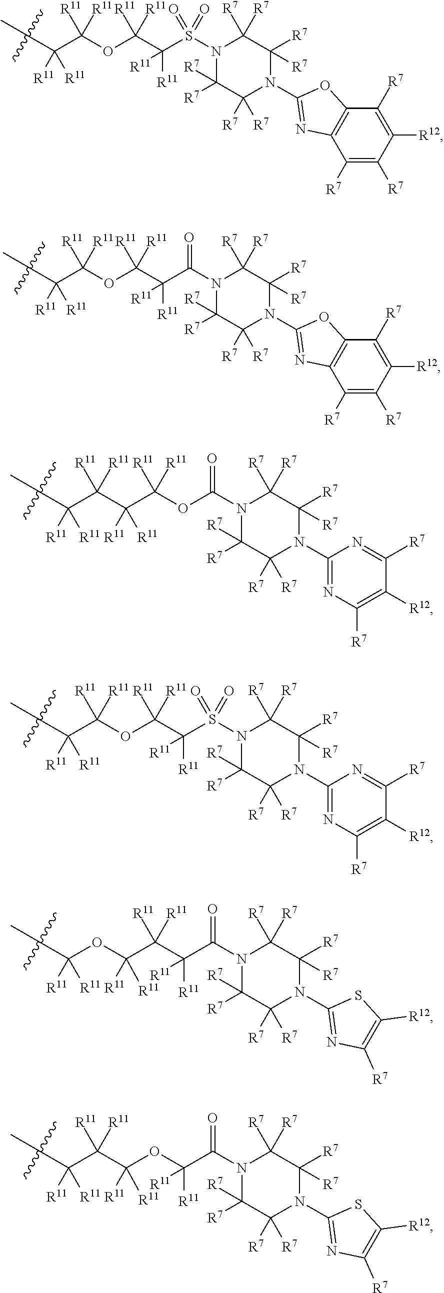 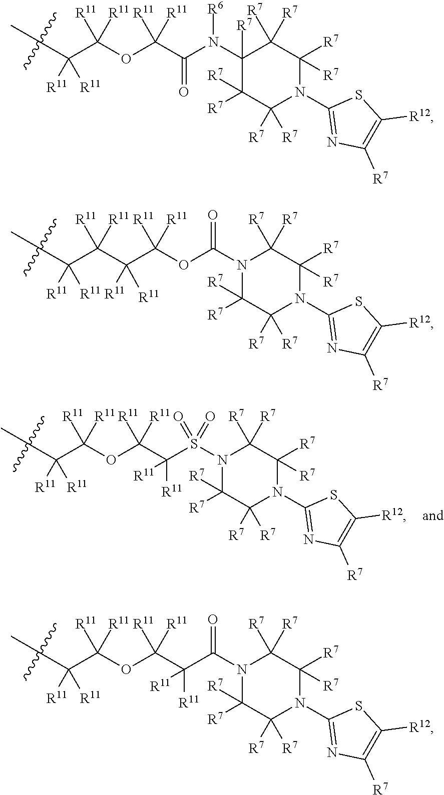
wherein R⁶, R⁷, R⁸, R¹¹ and R¹² are each independently H or a substituted or unsubstituted organic group.

10. A compound according to claim 9, which is selected from the following: 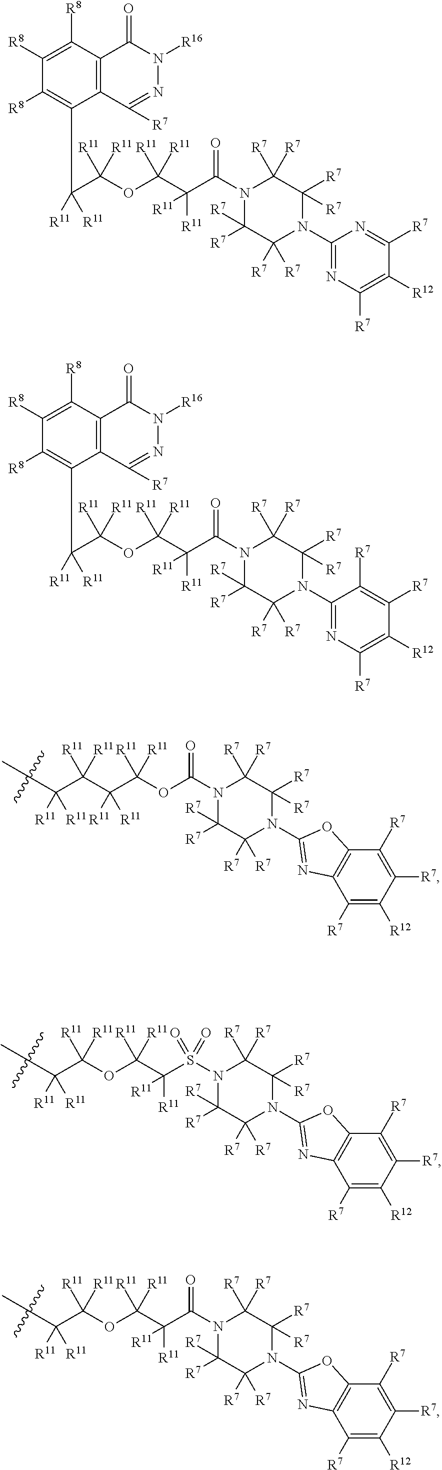 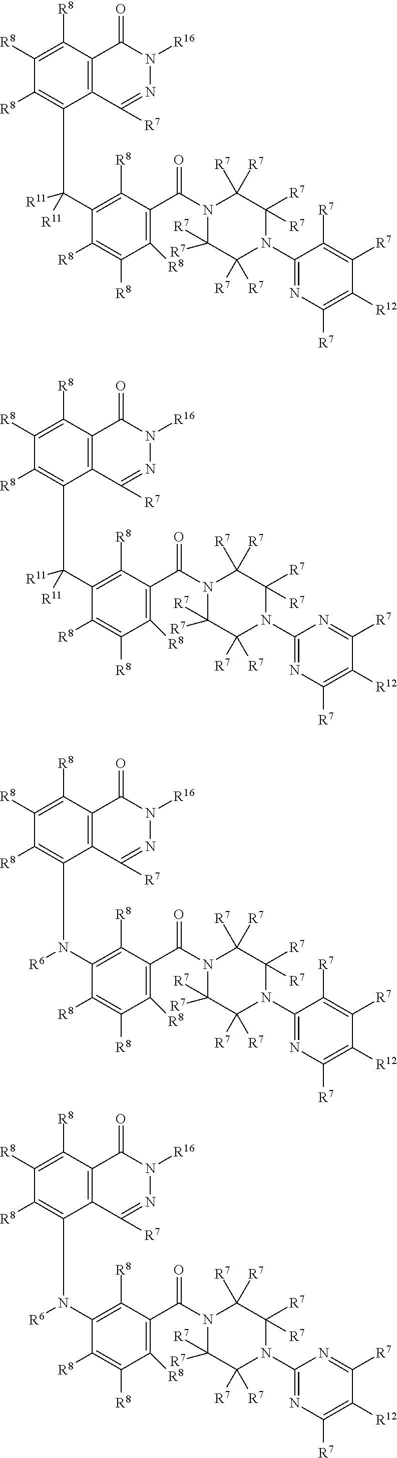 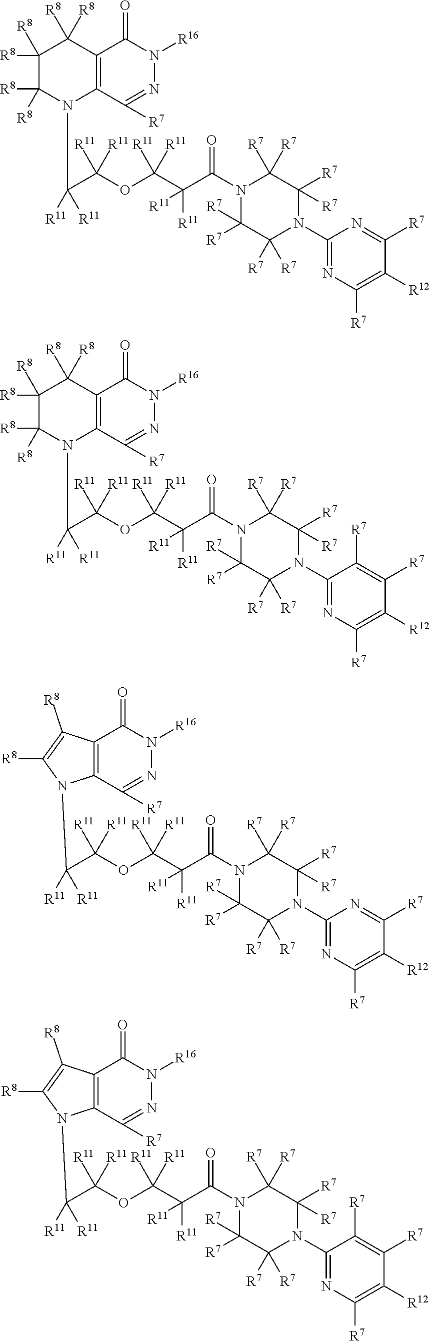 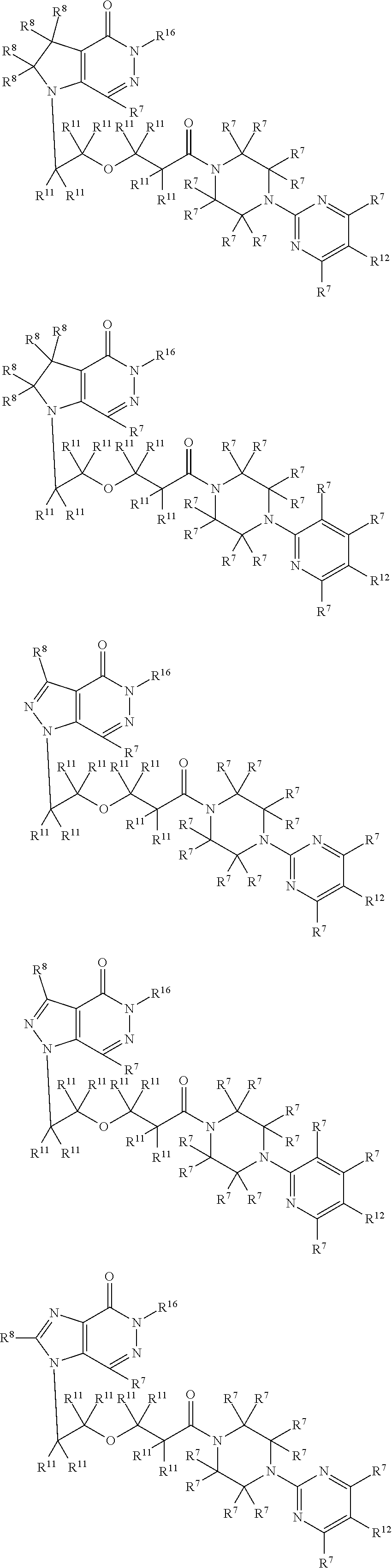 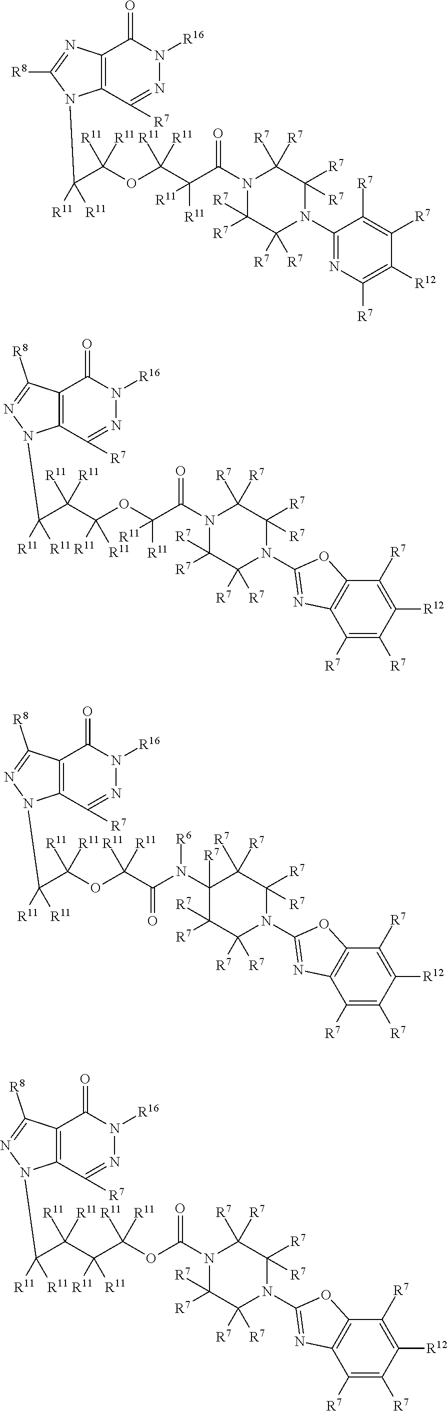 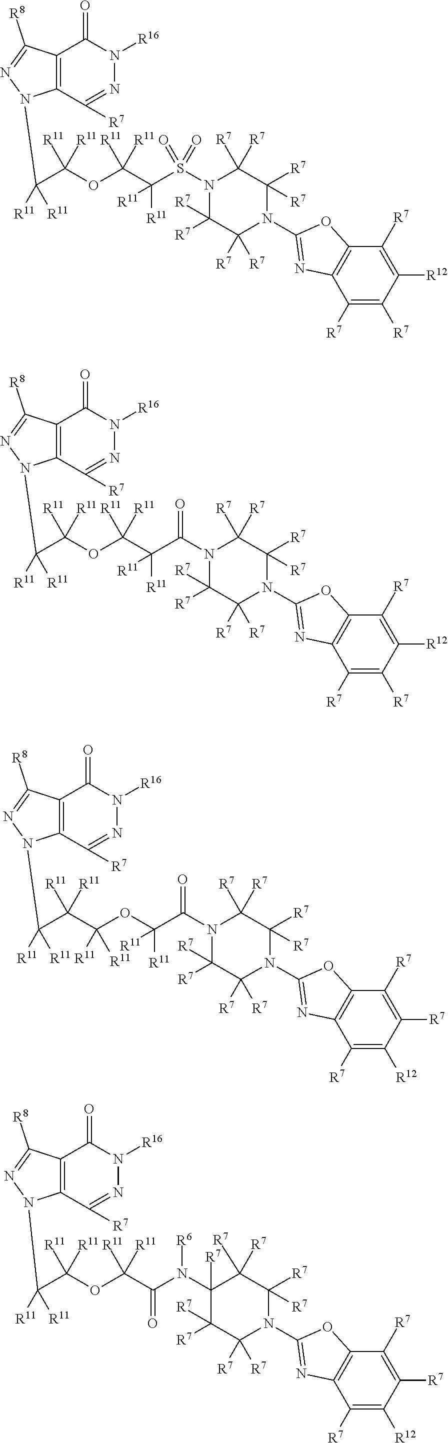 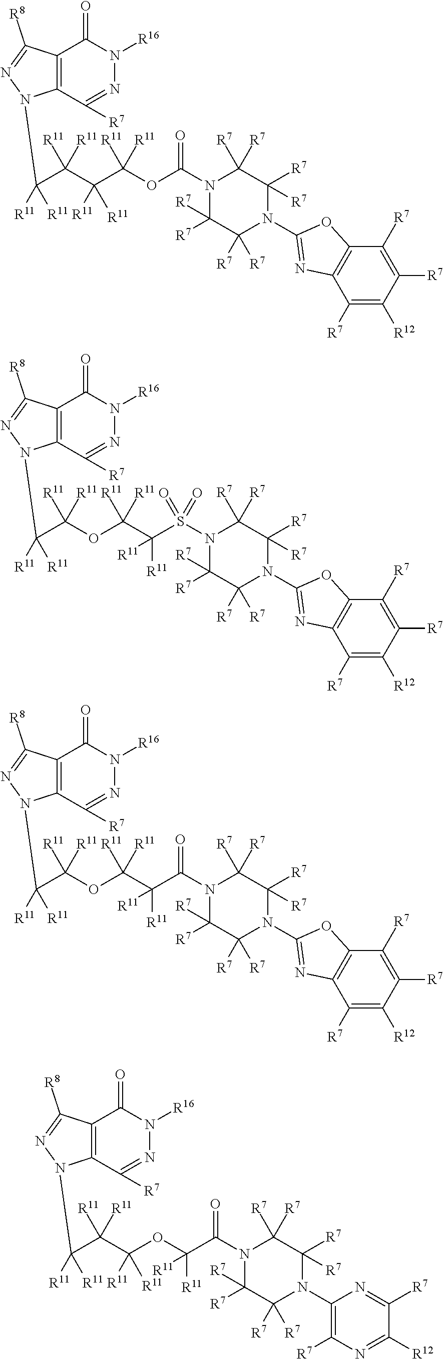 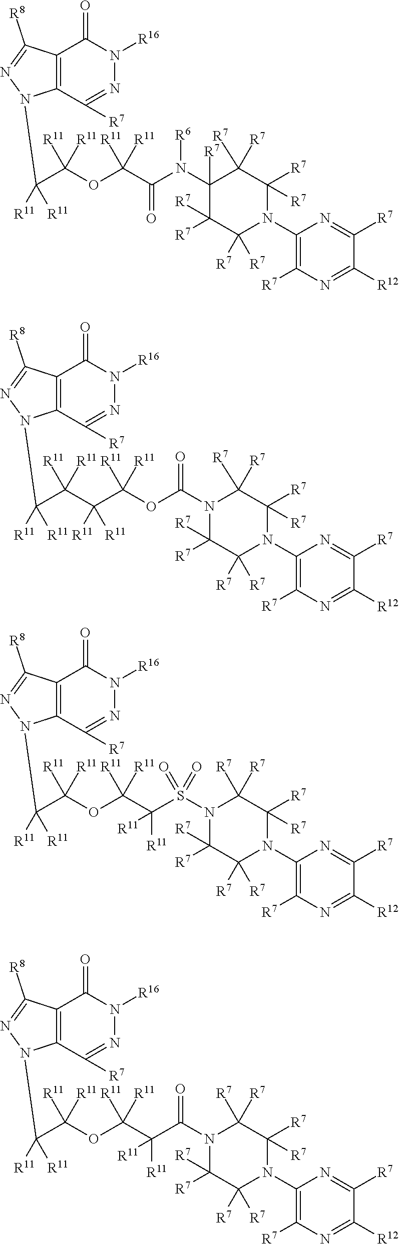 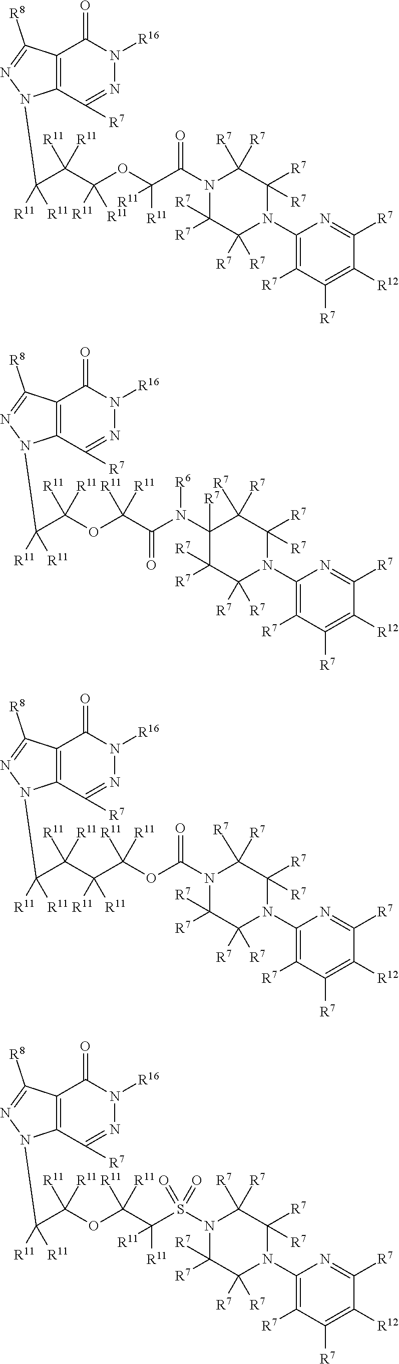 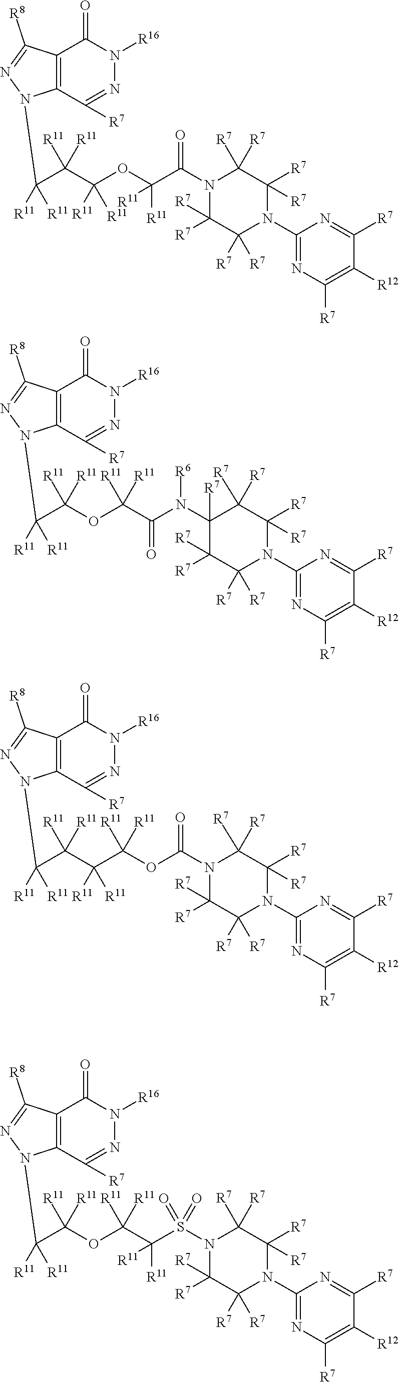 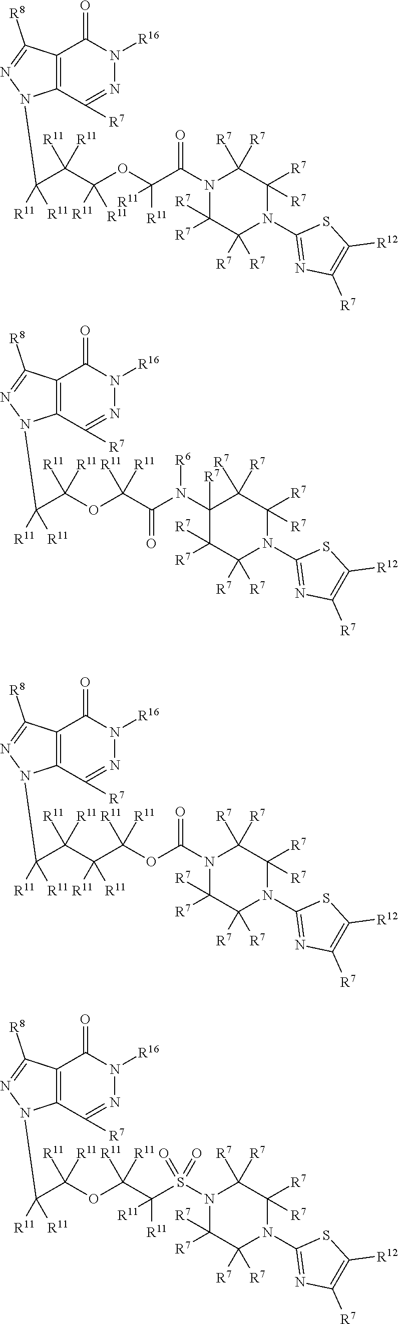 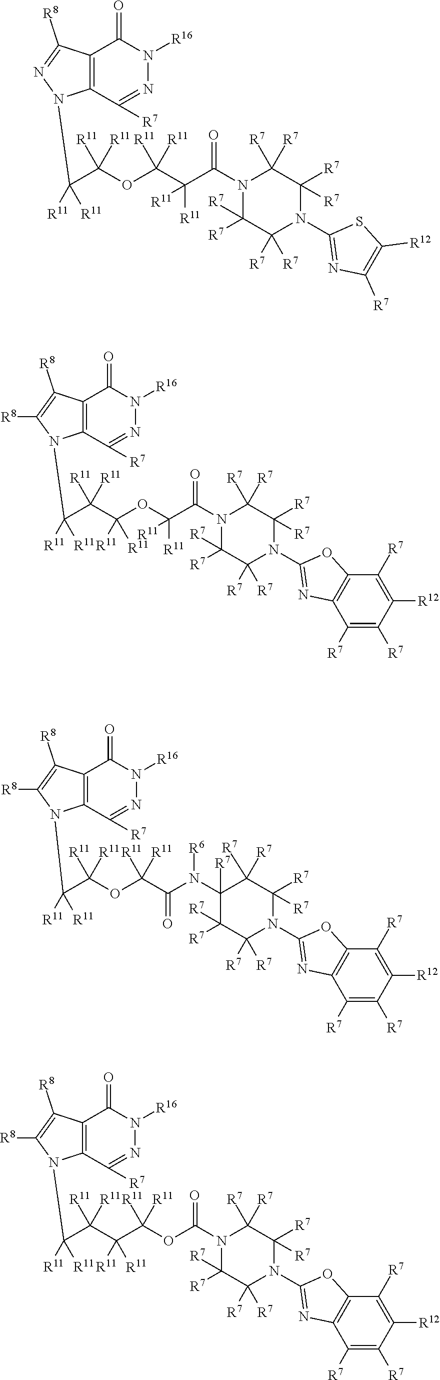 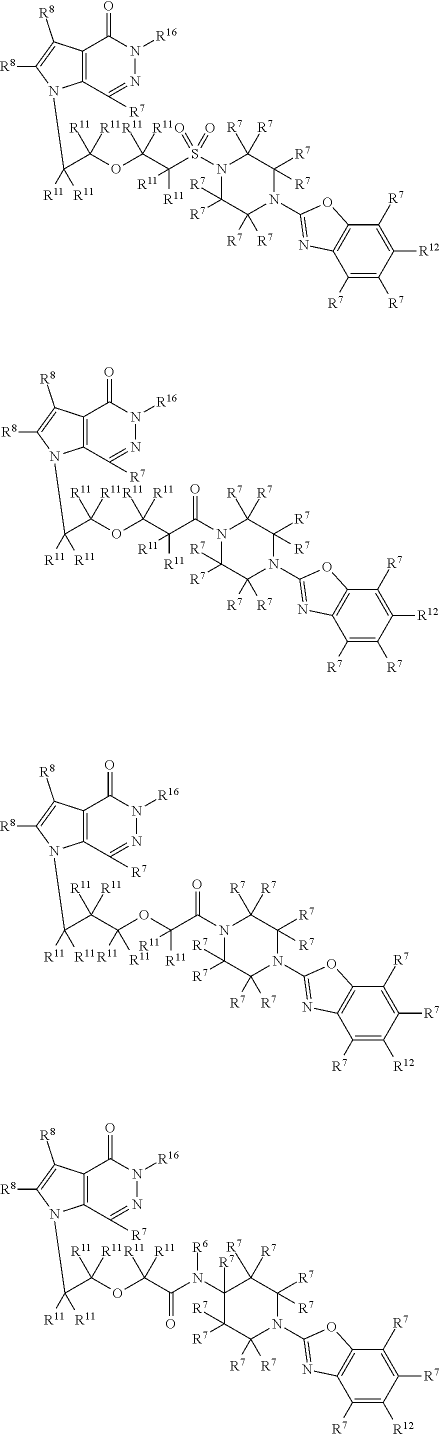 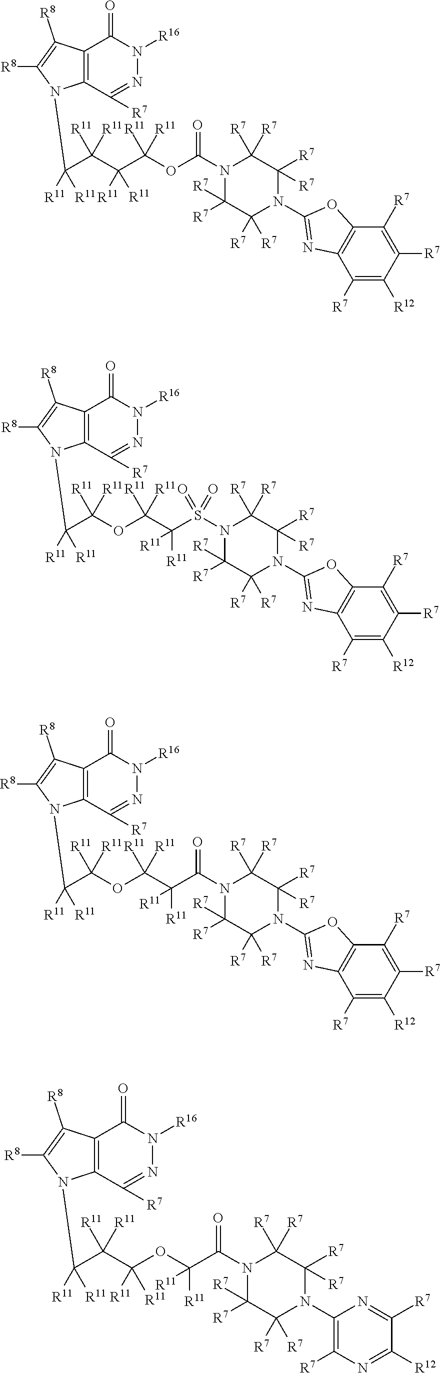 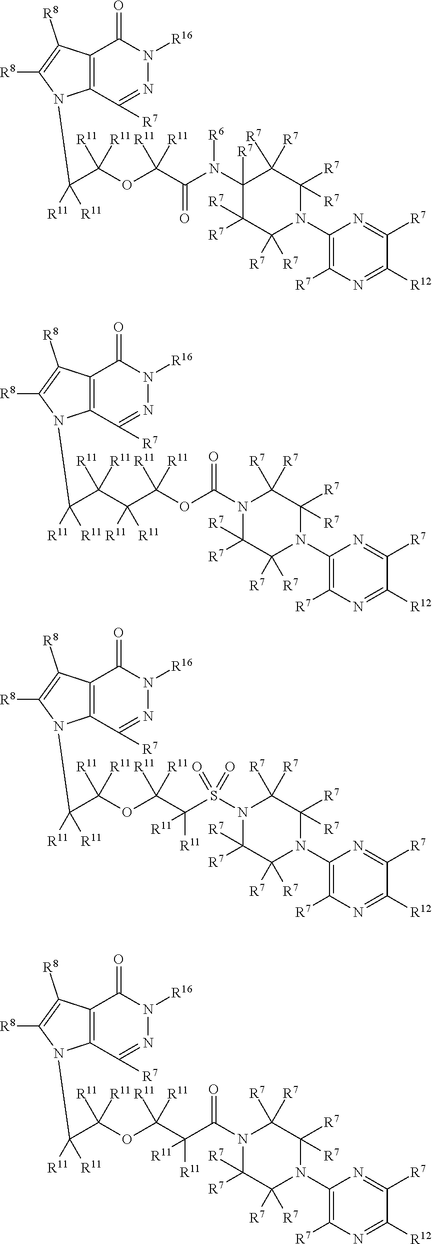 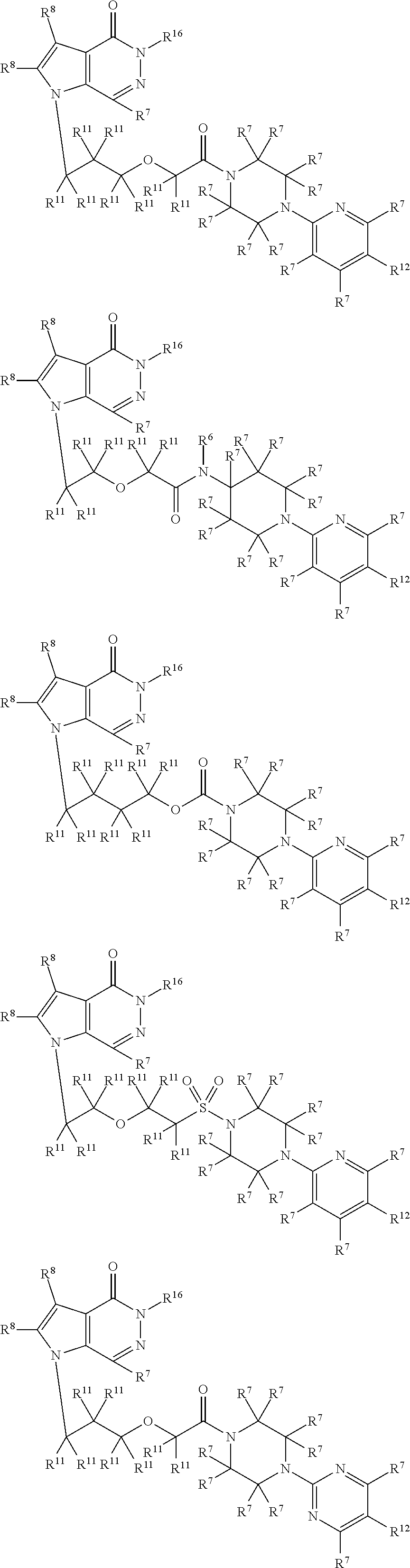 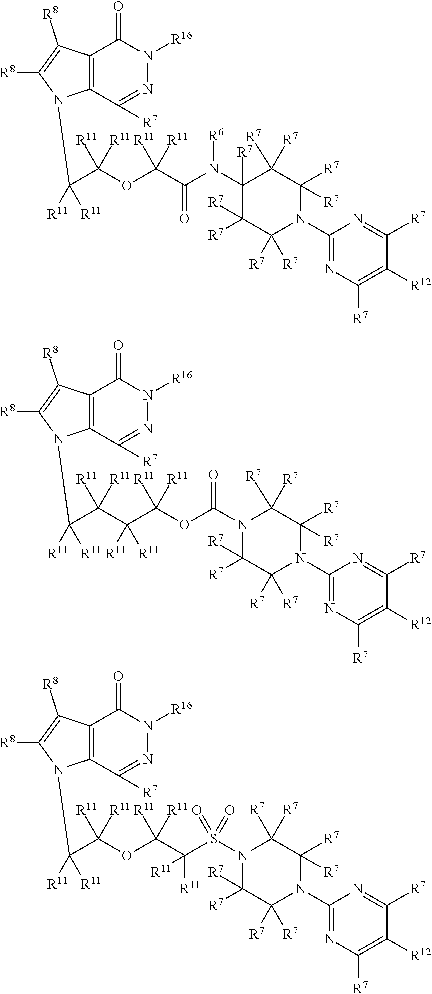 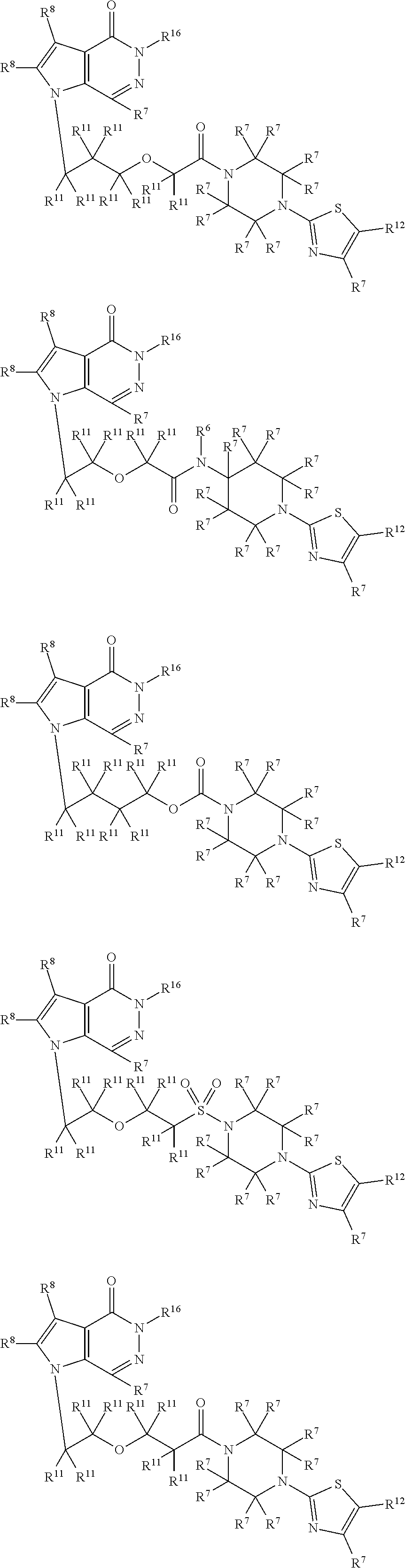 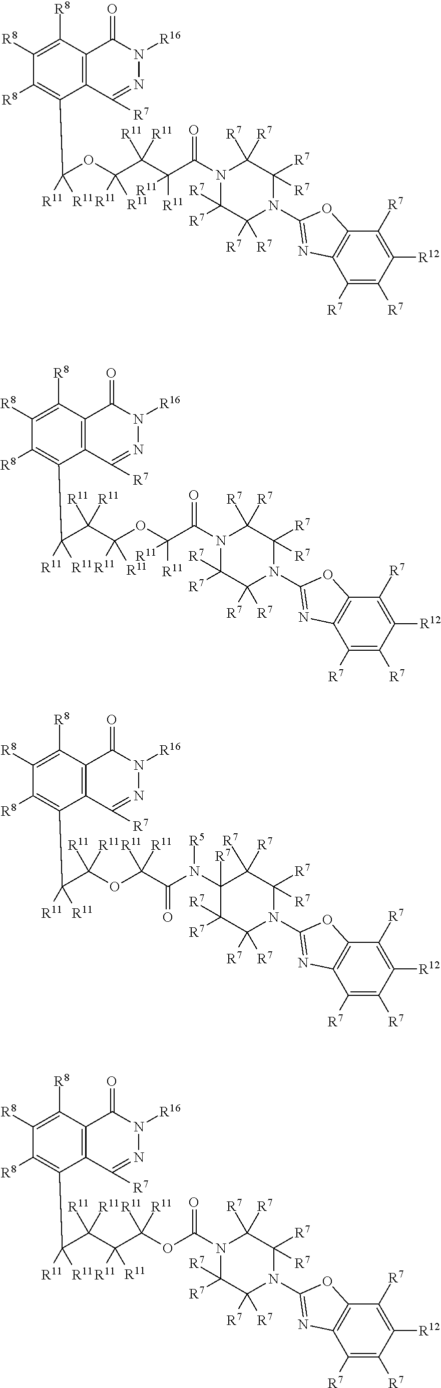 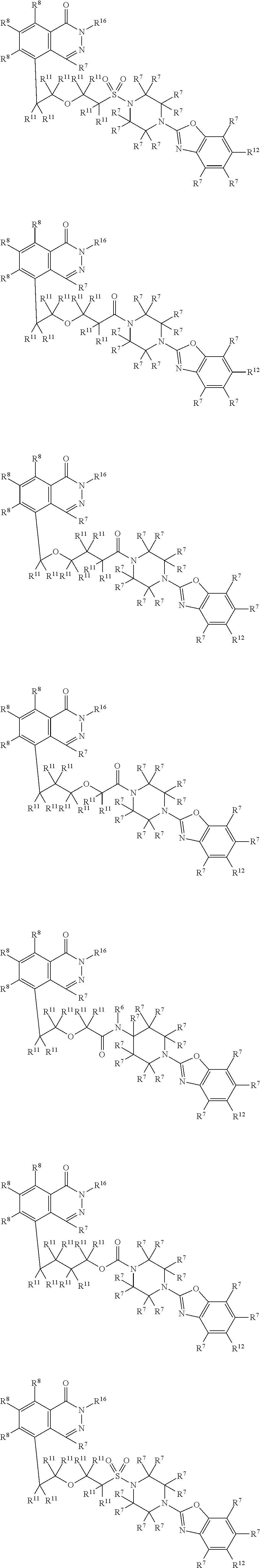 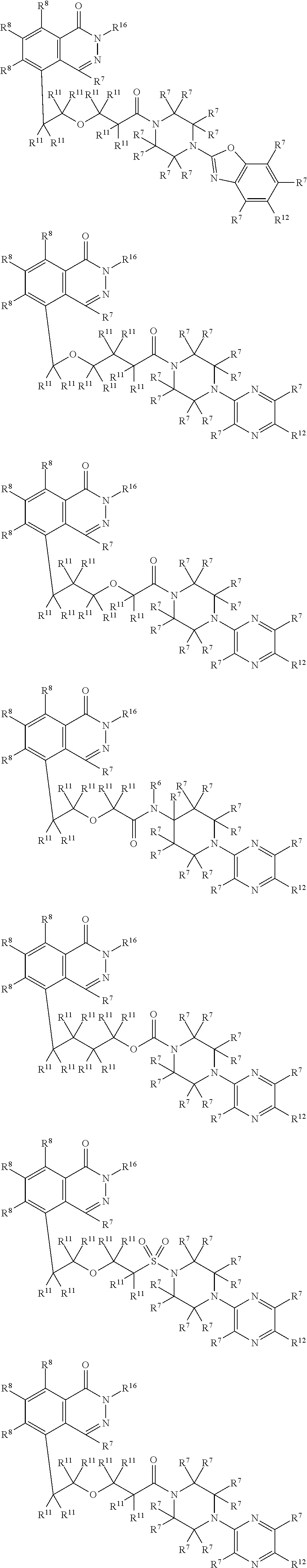 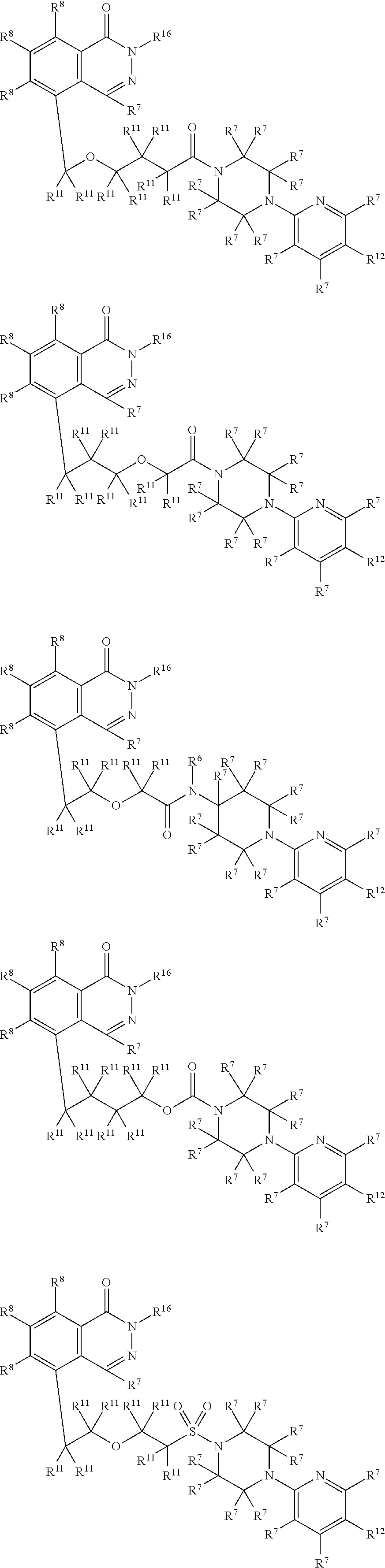 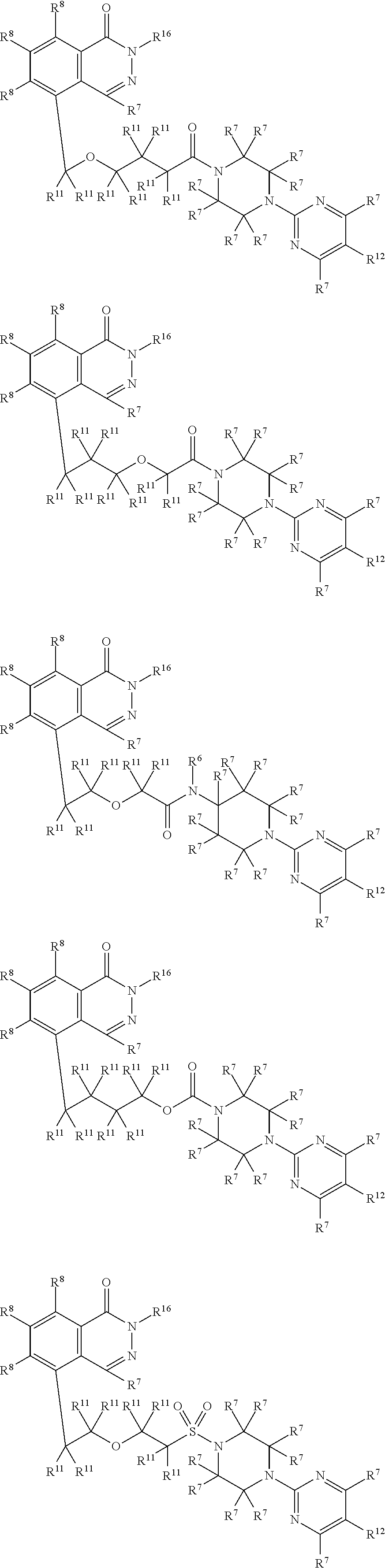 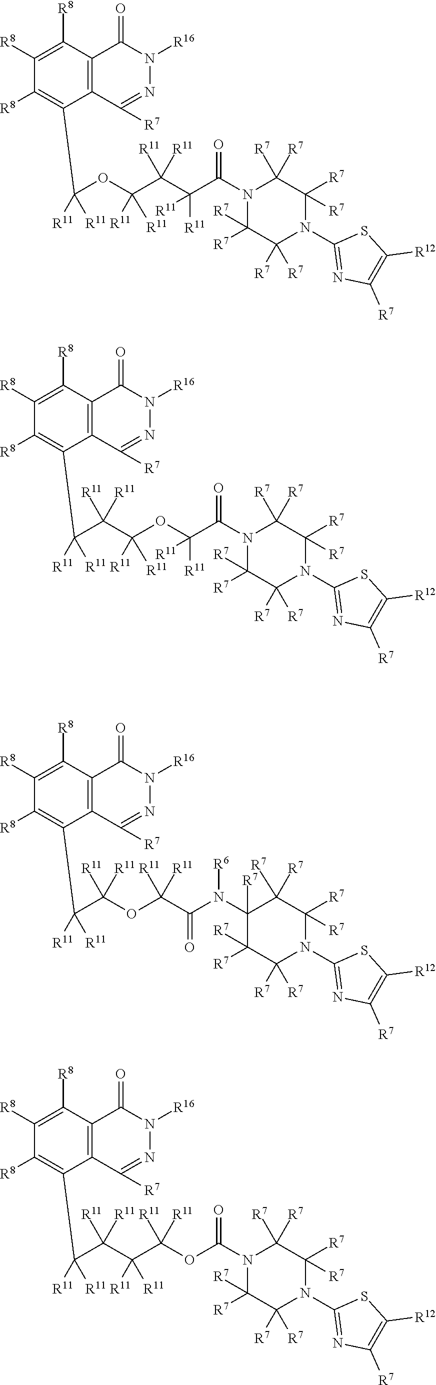 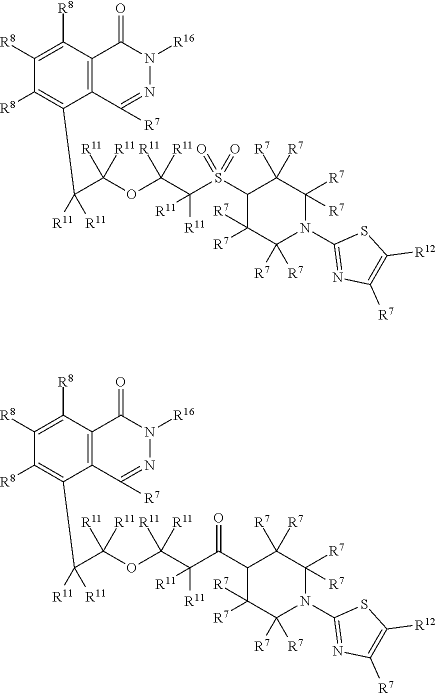
wherein R⁶, R⁷, R⁸, R¹¹ and R¹² are each independently H or a substituted or unsubstituted organic group.

11. A compound according to claim 1, which compound comprises the following formula:
wherein m is 1, 2, or 3;
and wherein R¹ may be attached to Z¹ by a single bond or a double bond and is a substituent of formula: 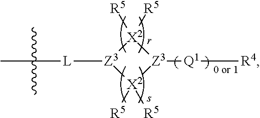
wherein L is a group selected from any of the following: 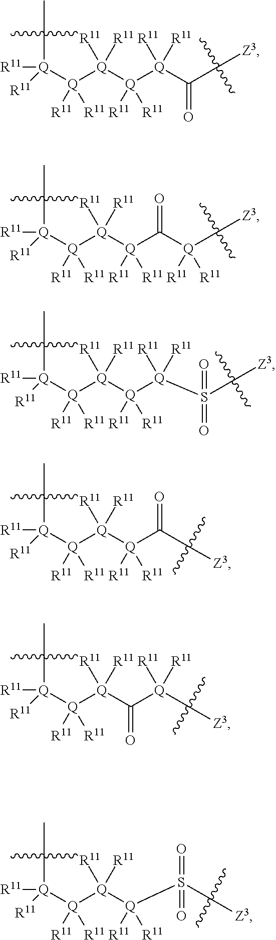 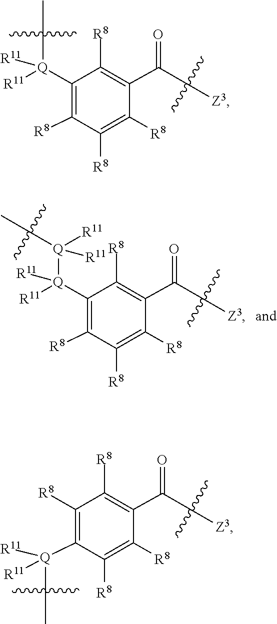
wherein each R⁸ is independently selected from H and a substituted or unsubstituted organic group; R¹¹ may be present or absent depending on the number of bonds and the valence of the Q atom comprising that R¹¹; and each R¹¹ is independently selected from H and a substituted or unsubstituted organic group;
wherein r is a number from 1 to 3; and s is independently a number from 1 to 3;
and wherein R⁴ is a group selected from any of the following: 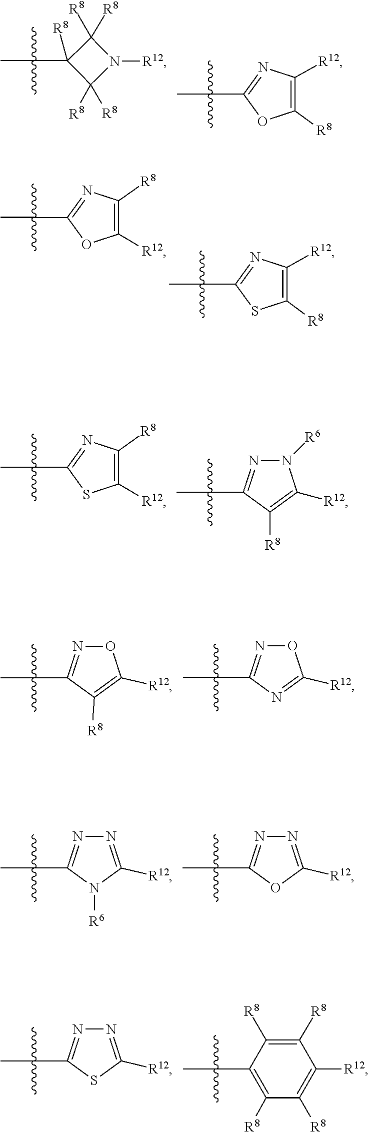 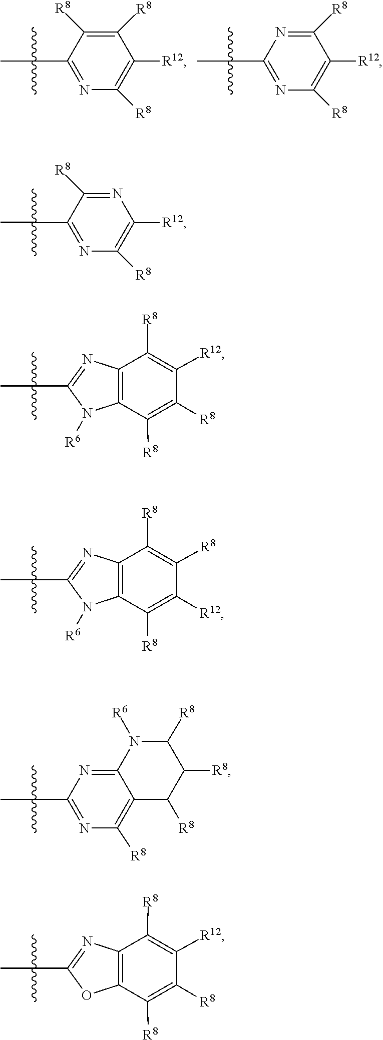 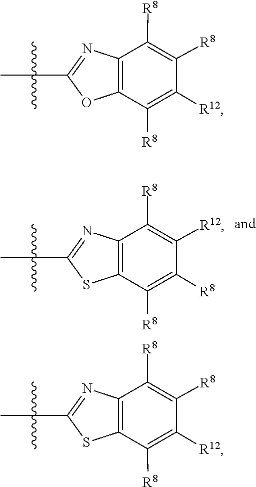
and wherein each R⁶ is independently selected from H and a substituted or unsubstituted organic group; and wherein R¹² is independently selected from H or a substituted or unsubstituted organic group and preferably a group selected from: —H, —CH₃, —CN, —CF₃, —CHF₂, —CH₂F, —OCF₃, —OMe, —CH₂CF₃, —CF₂CH₃, —OCHF₂, —OCH₂F, —F, —Cl, —Br, —I, —SO₂Me, —CONHMe, t-Bu, cyclopropyl and

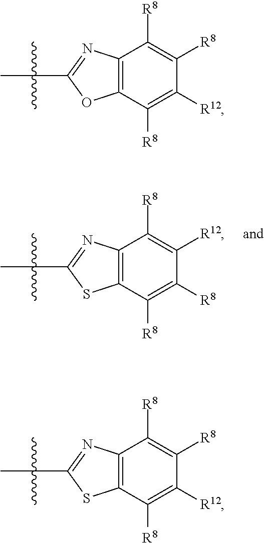

12. A compound according to claim 11, wherein R¹ has any of the following structures:

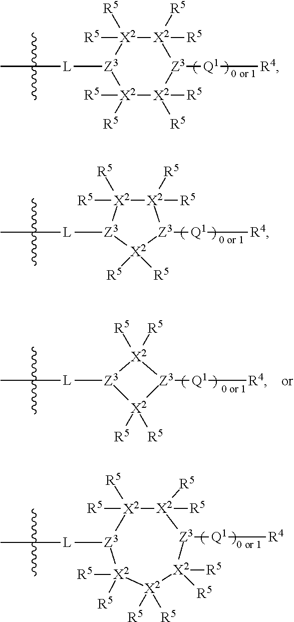

13. A compound according to claim 11, wherein R¹ has any of the following structures:

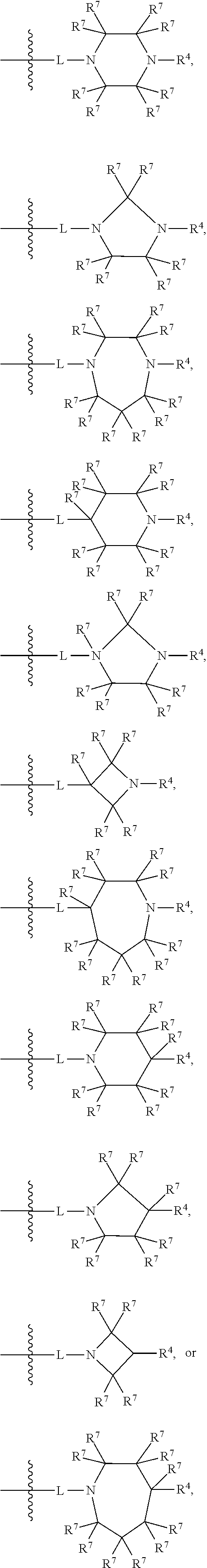

14. A compound according to claim 1, wherein ring B is selected from the following:

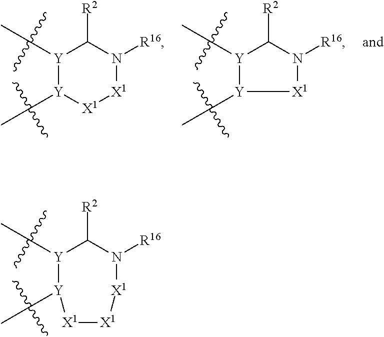

15. A compound according to claim 14, wherein ring B is selected from the following:

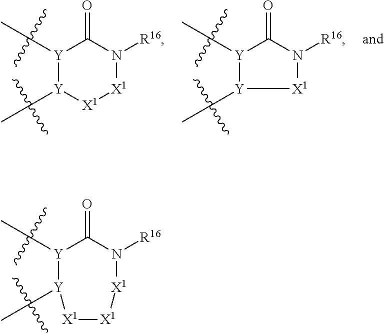

16. A compound according to claim 14, wherein ring B is selected from the following: 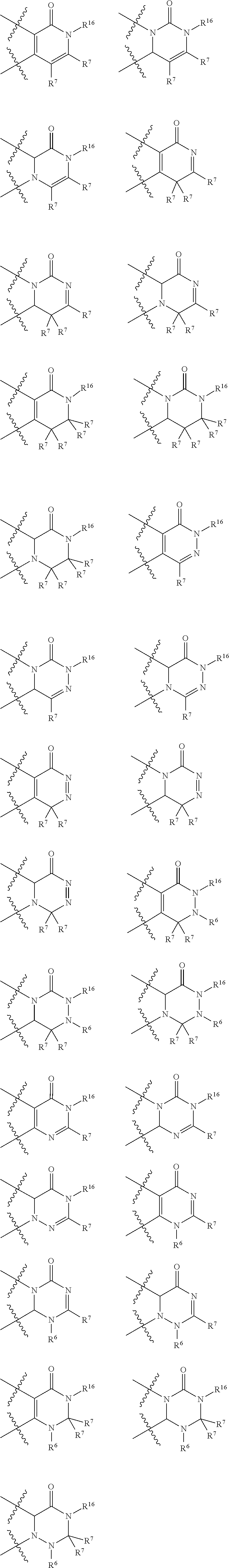 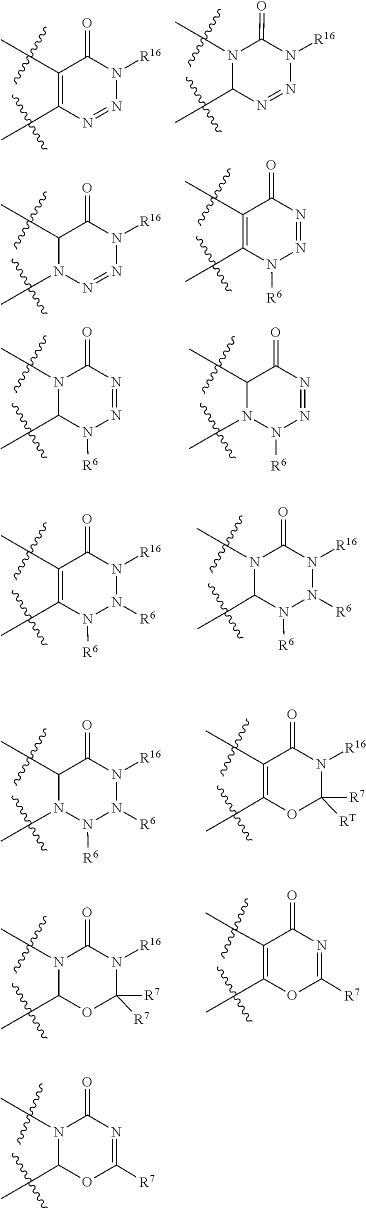 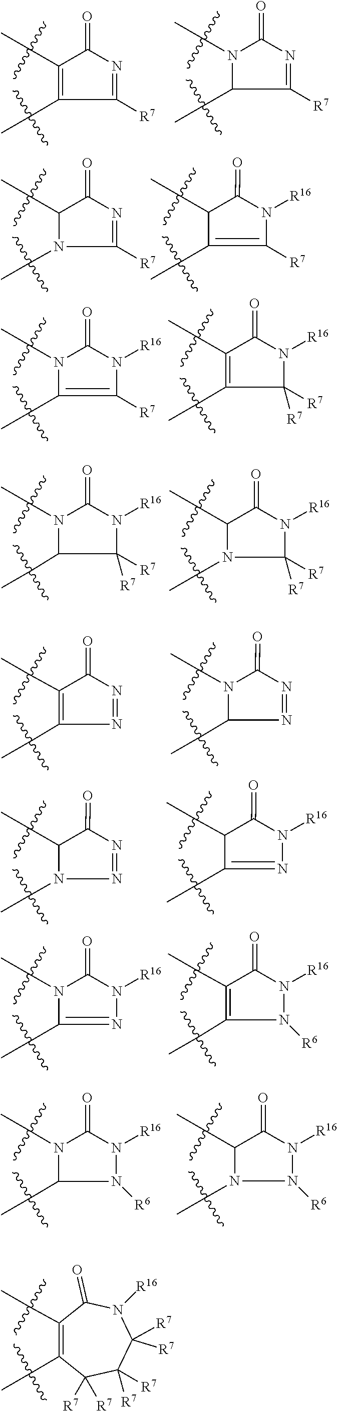 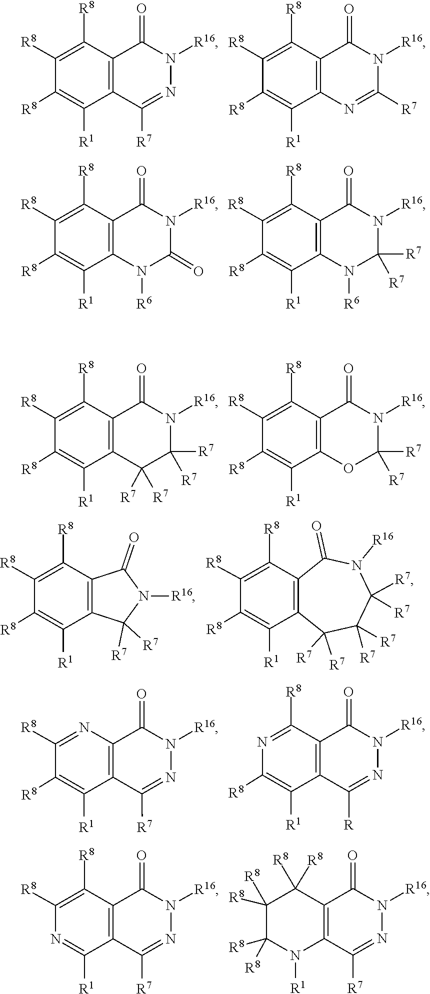 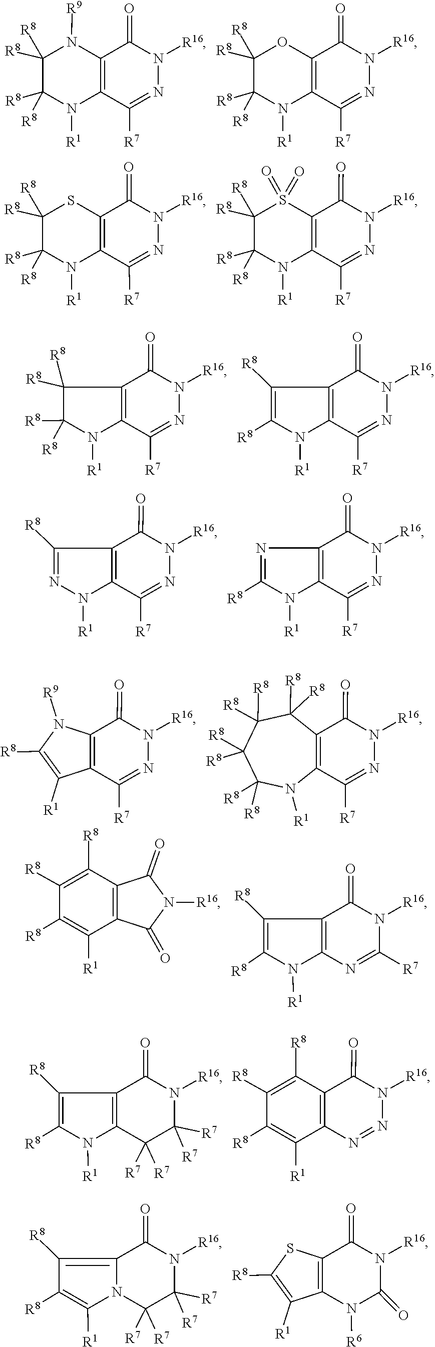 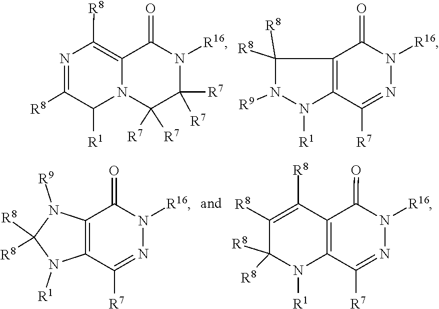
wherein R⁶ and R⁷ are independently selected from H or a substituted or unsubstituted organic group.

17. A compound according to claim 1, wherein ring A is selected from the following:

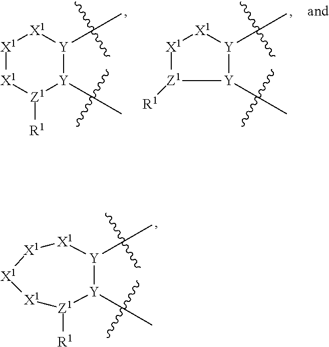

18. A compound according to claim 1, wherein ring A is selected from the following 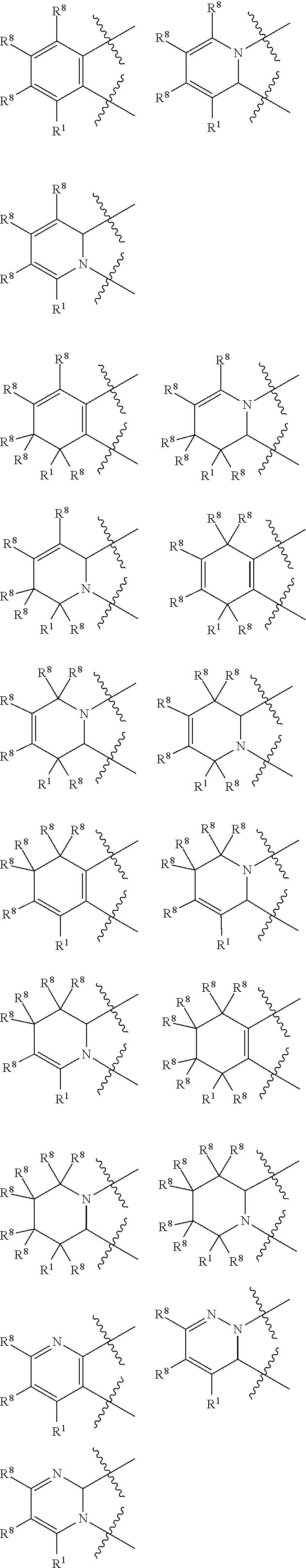 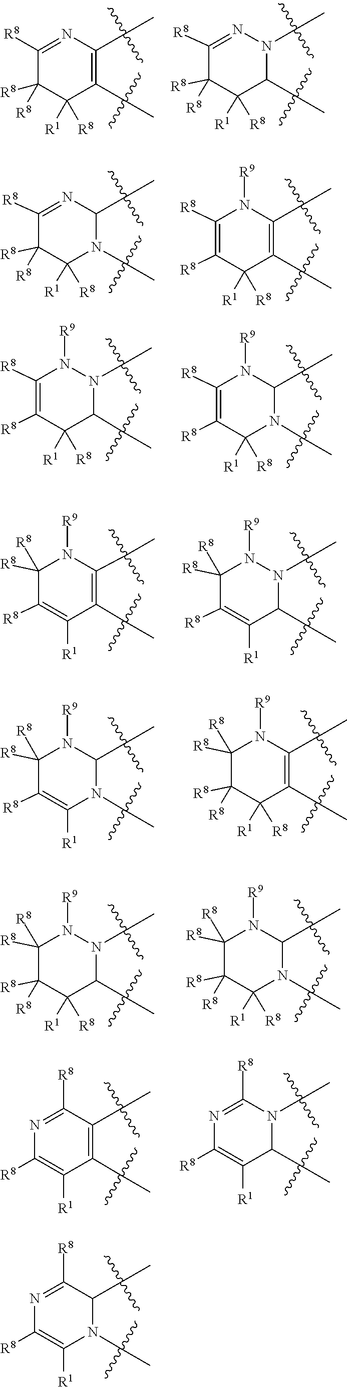 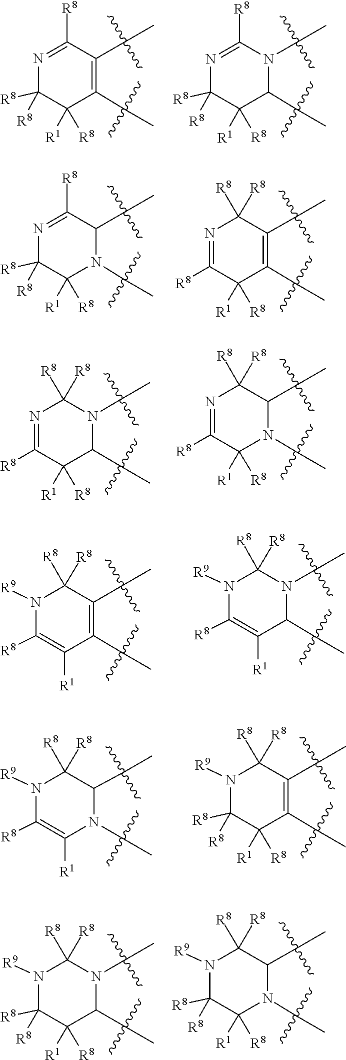 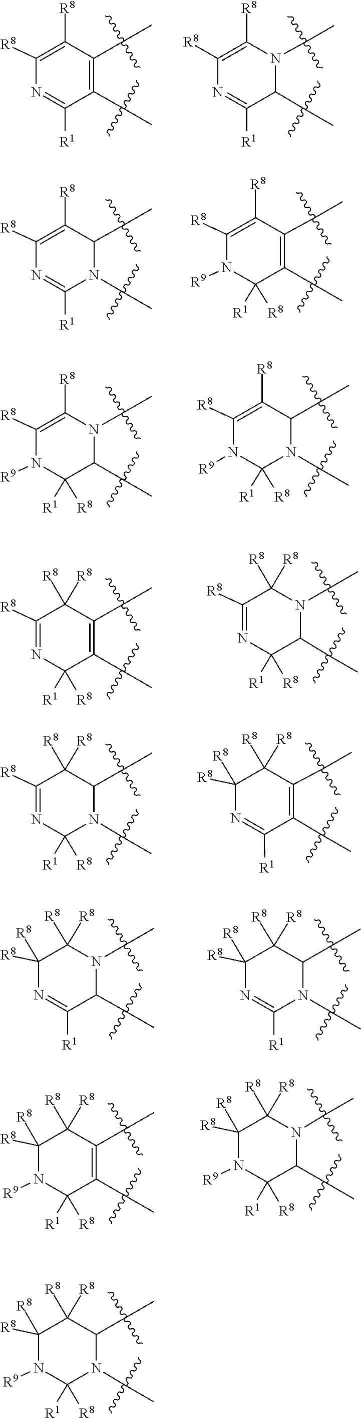 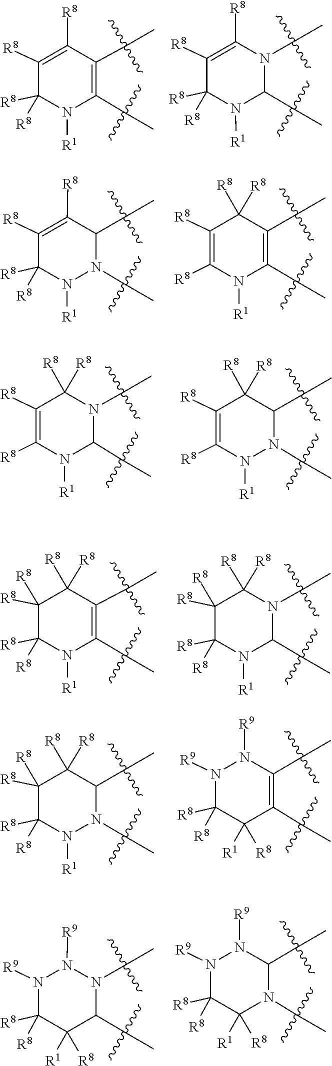 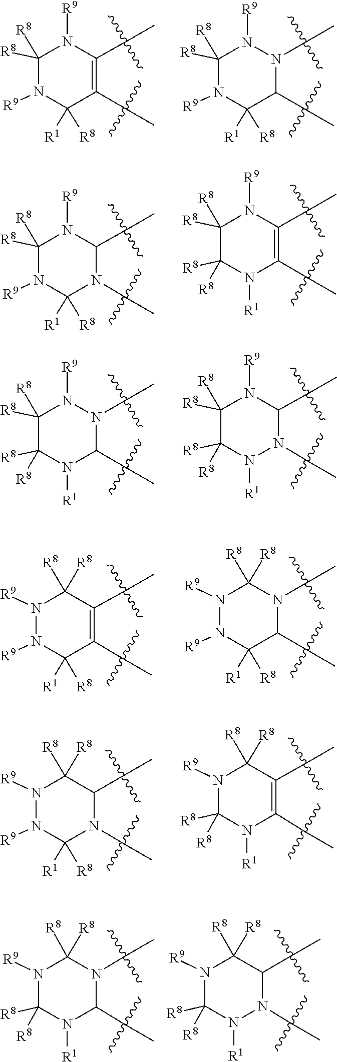 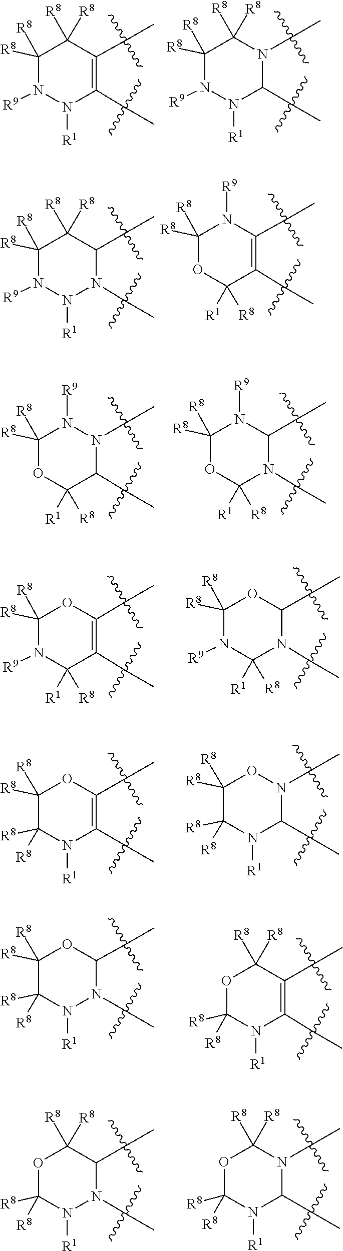 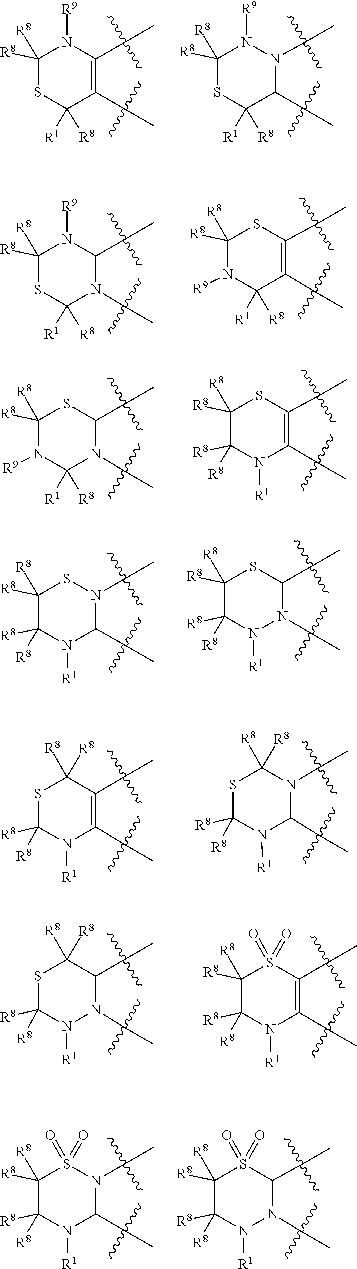 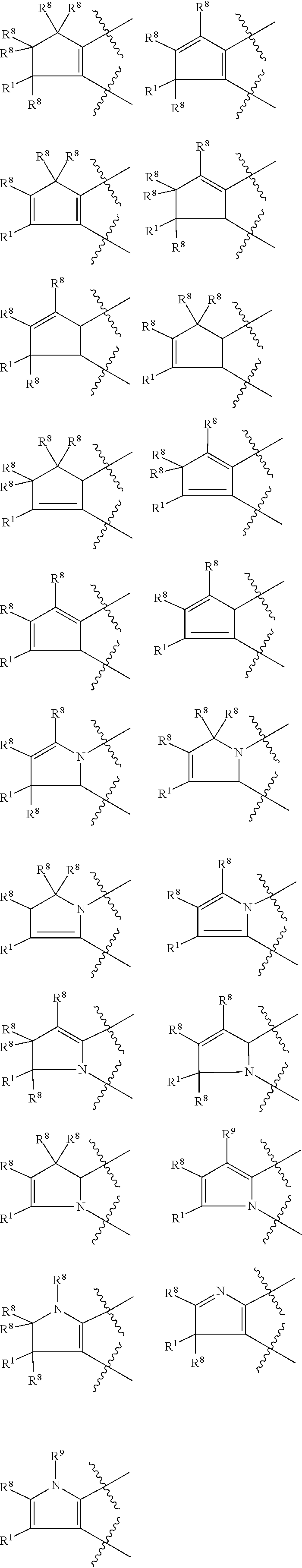 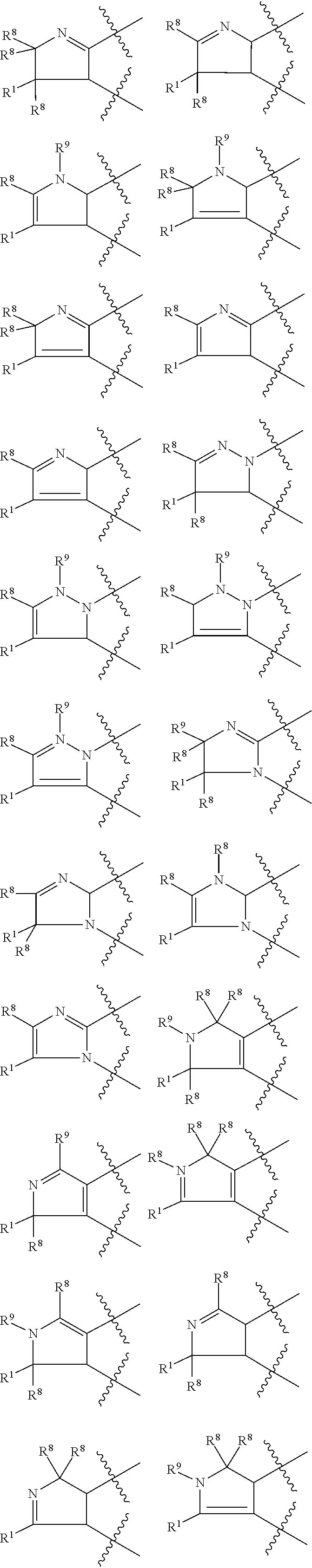 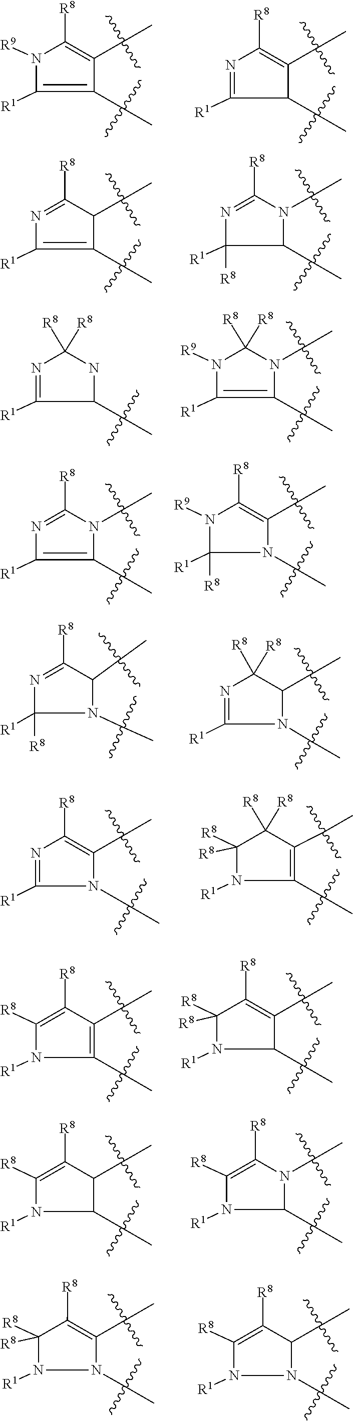 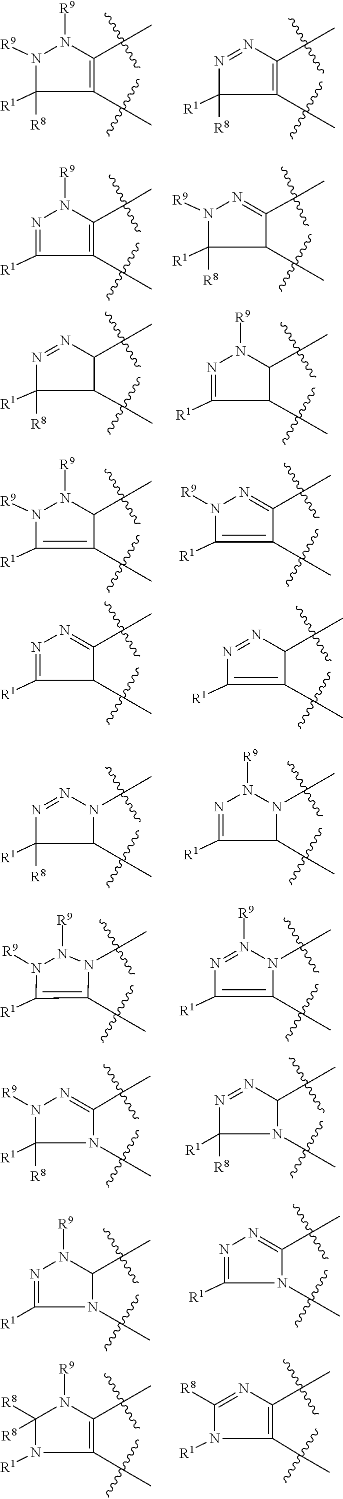 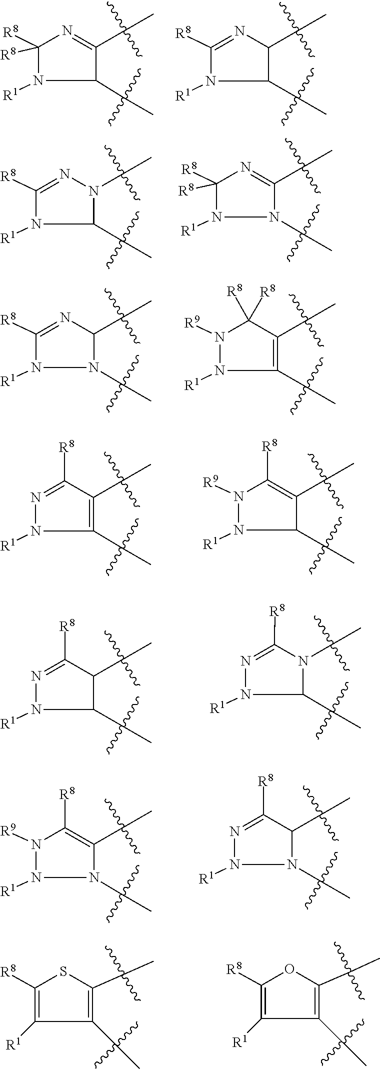
wherein R⁸ and R⁹ are independently selected from H and a substituted or unsubstituted organic group.

19. A compound according to claim 1, which compound comprises the following formula:

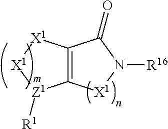

20. A compound according to claim 1, which is selected from the following:
wherein R⁶, R⁷, R⁸ and R⁹ are each independently H or a substituted or unsubstituted organic group.

21. A compound according to claim 1, wherein R⁵, R⁷, R⁸, R¹¹ and R¹² are each independently selected from H and a group selected from the following groups:
deuterium
a halogen (such as —F, —Cl, —Br and —I);
a substituted or unsubstituted linear or branched C₁-C₆ alkyl group (such as Me, Et, Pr, i-Pr, n-Bu, i-Bu, t-Bu, pentyl and hexyl);
a substituted or unsubstituted linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a substituted or unsubstituted linear or branched C₁-C₆ halogenated alkyl group (such as —CH₂F, —CHF₂, —CH₂CH₂F, —CH₂Cl, —CH₂Br, —CH₂₁, —CF₃, —CCl₃, —CBr₃, —Cl₃, —CH₂CF₃, —CH₂CCl₃, —CH₂CBr₃, and —CH₂Cl₃);
NH₂ or a substituted or unsubstituted linear or branched primary secondary or tertiary C₁-C₆ amine group (such as —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂-NEtH, —CH₂-NEtMe, —CH₂-NEt₂, —CH₂—NPrH, —CH₂—NPrMe, and —CH₂—NPrEt);
a substituted or unsubstituted amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph,
a substituted or unsubstituted cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a substituted or unsubstituted cyclic C₃-C₈ alkyl group (such as cyclopropyl, cyclobutyl, cyclopentyl, cyclohexyl, cycloheptyl and cyclooctyl);
an —OH group or a substituted or unsubstituted linear or branched C₁-C₆ alcohol group (such as —CH₂OH, —CH₂CH₂OH, —CH(CH₃)CH₂OH, —C(CH₃)₂OH, —CH₂CH₂CH₂OH, —CH₂CH₂CH₂CH₂OH, —CH(CH₃)CH₂CH₂OH, —CH(CH₃)CH(CH₃)OH, —CH(CH₂CH₃)CH₂OH, —C(CH₃)₂CH₂OH, —CH₂CH₂CH₂CH₂CH₂OH, and —CH₂CH₂CH₂CH₂CH₂CH₂OH); —a substituted or unsubstituted linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a substituted or unsubstituted linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a substituted or unsubstituted linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a substituted or unsubstituted linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a substituted or unsubstituted linear or branched C₁-C₇ amino carbonyl group (such as —NH—CO-Me, —NH—CO-Et, —NH—CO—Pr, —NH—CO-Bu, —NH—CO-pentyl, —NH—CO-hexyl, —NH—CO-Ph, —NMe-CO-Me, —NMe-CO-Et, —NMe-CO—Pr, —NMe-CO-Bu, —NMe-CO-pentyl, —NMe-CO-hexyl, —NMe-CO-Ph;
a substituted or unsubstituted linear or branched C₁-C₇ alkoxy or aryloxy group (such as —OMe, —OEt, —OPr, —O-i-Pr, —O-n-Bu, —O-i-Bu, —O-t-Bu, —O-pentyl, —O— hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a substituted or unsubstituted linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a substituted or unsubstituted sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃), —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe;
a substituted or unsubstituted aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a substituted or unsubstituted aromatic group (such as Ph-, 2-F-Ph-, 3-F-Ph-4-F-Ph-, 2-Cl-Ph-, 3-Cl-Ph-, 4-Cl-Ph-, 2-Br-Ph-, 3-Br-Ph-, 4-Br-Ph-, 2-1-Ph-, 3-1-Ph, 4-1-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-);
a saturated or unsaturated, substituted or unsubstituted, heterocyclic group including an aromatic heterocyclic group and/or a non-aromatic heterocyclic group (such as pyrrole-1-yl, pyrrole-2-yl, pyrrole-3-yl, pyrazole-1-yl, pyrazole-3-yl, pyrazole-4-yl, pyrazole-5-yl, imidazole-1-yl, imidazole-2-yl, imidazole-4-yl, imidazole-5-yl, 1,2,3-triazole-1-yl, 1,2,3-triazole-4-yl, 1,2,3-triazole-5-yl, 1,2,4-triazole-1-yl, 1,2,4-triazole-3-yl, 1,2,4-triazole-5-yl, pyridin-2-yl, pyridin-3-yl, pyridin-4-yl, pyridazine-3-yl, pyridazine-4-yl, pyrimidin-2-yl, pyrimidin-4-yl, pyrimidin-5-yl, pyrimidin-6-yl, pyrazine-2-yl, pyrrolidine-1-yl, pyrrolidine-2-yl, pyrrolidine-3-yl, piperidine-1-yl, piperidine-2-yl, piperidine-3-yl, piperidine-4-yl, 2-azapiperidine-1-yl, 2-azapiperidine-3-yl, 2-azapiperidine-4-yl, 3-azapiperidine-1-yl, 3-azapiperidine-2-yl, 3-azapiperidine-4-yl, 3-azapiperidine-5-yl, piperazine-1-yl, piperazine-2-yl, furan-2-yl, furan-3-yl, pyran-2-yl, pyran-3-yl, pyran-4-yl, 2-azapyran-2-yl, 2-azapyran-3-yl, 2-azapyran-4-yl, 2-azapyran-5-yl, 2-azapyran-6-yl, 3-azapyran-2-yl, 3-azapyran-4-yl, 3-azapyran-5-yl, 3-azapyran-6-yl, 4-azapyran-2-yl, 4-azapyran-3-yl, 4-azapyran-4-yl, 4-azapyran-5-yl, 4-azapyran-6-yl, oxetan-2-yl, oxetan-3-yl, tetrahydrofuran-2-yl, tetrahydrofuran-3-yl, 2-aza-tetrahydrofuran-2-yl, 2-aza-tetrahydrofuran-3-yl, 2-aza-tetrahydrofuran-4-yl, 2-aza-tetrahydrofuran-5-yl, 3-aza-tetrahydrofuran-2-yl, 3-aza-tetrahydrofuran-3-yl, 3-aza-tetrahydrofuran-4-yl, 3-aza-tetrahydrofuran-5-yl, tetrahydropyran-2-yl, tetrahydropyran-3-yl, tetrahydropyran-4-yl, 2-aza-tetrahydropyran-2-yl, 2-aza-tetrahydropyran-3-yl, 2-aza-tetrahydropyran-4-yl, 2-aza-tetrahydropyran-5-yl, 2-aza-tetrahydropyran-6-yl, 3-aza-tetrahydropyran-2-yl, 3-aza-tetrahydropyran-3-yl, 3-aza-tetrahydropyran-4-yl, 3-aza-tetrahydropyran-5-yl, 3-aza-tetrahydropyran-6-yl, morpholine-2-yl, morpholine-3-yl, morpholine-4-yl, thiophen-2-yl, thiophen-3-yl, isothiazole-3-yl, isothiazole-4-yl, isothiazole-5-yl, thiazole-2-yl, thiazole-4-yl, thiazole-5-yl, thiopyran-2-yl, thiopyran-3-yl, thiopyran-4-yl, 2-azathiopyran-2-yl, 2-azathiopyran-3-yl, 2-azathiopyran-4-yl, 2-azathiopyran-5-yl, 2-azathiopyran-6-yl, 3-azathiopyran-2-yl, 3-azathiopyran-4-yl, 3-azathiopyran-5-yl, 3-azathiopyran-6-yl, 4-azathiopyran-2-yl, 4-azathiopyran-3-yl, 4-azathiopyran-4-yl, 4-azathiopyran-5-yl, 4-azathiopyran-6-yl, thiolane-2-yl, thiolane-3-yl, thiane-2-yl, thiane-3-yl, thiane-4-yl, oxazol-2-yl, oxazol-4-yl, oxazol-5-yl, isoxazol-3-yl, isoxazol-4-yl, isoxazol-5-yl, furazan-3-yl, (1,3,4-oxadiazol)-2-yl, (1,3,4-oxadiazol)-5-yl, (1,2,4-oxadiazol)-3-yl, (1,2,4-oxadiazol)-5-yl; and tetrazole-1-yl, tetrazole-2-yl, tetrazole-5-yl);
where there are two R groups attached to the same atom, they may together form a group which is double bonded to that atom, (such as a carbonyl group (═O) or an alkene group (═C(R′)₂) wherein each R′ group is the same or different and is H or an organic group, preferably H or a straight or branched C₁-C₆ alkyl group); and
R⁷ and R⁸ may also be independently selected from a nitrile group.

22. A compound according to claim 21, wherein R⁵ is independently selected from H, deuterium, a halogen (such as —F, —Cl, —Br, and —I, preferably F), a substituted or unsubstituted C₁-C₆ alkyl group, a substituted or unsubstituted linear or branched C₁-C₆ halogenated alkyl group, an —OH group or a substituted or unsubstituted linear or branched C₁-C₆ alcohol group, an —NH₂ group or a substituted or unsubstituted C₁-C₆ amino group and a substituted or unsubstituted C₁-C₆ alkoxy group; or wherein there are two R⁵ groups on the same atom which together form a carbonyl group.

23. A compound according to claim 21, wherein R⁷ and R⁸ are each independently selected from H, deuterium, a halogen (such as —F, —Cl, —Br, and —I), a substituted or unsubstituted C₁-C₆ alkyl or cycloalkyl group, a substituted or unsubstituted linear or branched C₁-C₆ halogenated alkyl group, an —OH group or a substituted or unsubstituted linear or branched C₁-C₆ alcohol group, an —NH₂ group or a substituted or unsubstituted C₁-C₆ amino group, a substituted or unsubstituted C₁-C₆ alkoxy group, and a nitrile group; or wherein there are two R⁷ or R⁸ groups on the same atom which together form a carbonyl group.

24. A compound according to claim 21, wherein R¹¹ is selected from H, deuterium a halogen (such as —F, —Cl, —Br, and —I, preferably —F), a substituted or unsubstituted C₁-C₆ alkyl group, a substituted or unsubstituted linear or branched C₁-C₆ halogenated alkyl group (preferably CF₃), an —NH₂ group or a substituted or unsubstituted C₁-C₆ amino group, an —OH group or a substituted or unsubstituted linear or branched C₁-C₆ alcohol group and a substituted or unsubstituted C₁-C₆ alkoxy group.

25. A compound according to claim 21, wherein R¹² is selected from: —H, —CH₃, —CN, —CF₃, —CHF₂, —CH₂F, —OCF₃, —OMe, —CH₂CF₃, —CF₂CH₃, —OCHF₂, —OCH₂F, —F, —Cl, —Br, —I, —SO₂Me, —CONHMe, t-Bu, cyclopropyl and

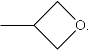

26. A compound according to claim 1, wherein R³, R⁶ and R⁹ are each independently selected from H and a group selected from the following groups:
a substituted or unsubstituted linear or branched C₁-C₆ alkyl group (such as Me, Et, Pr, i-Pr, n-Bu, i-Bu, t-Bu, pentyl and hexyl);
a substituted or unsubstituted linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a substituted or unsubstituted linear or branched C₁-C₆ halogenated alkyl group (such as —CH₂F, —CH₂CF₃ and —CH₂CH₂F);
a substituted or unsubstituted cyclic amine or amido group (such as pyrrolidin-3-yl, piperidin-3-yl, piperidin-4-yl, 2-keto-pyrrolidinyl, 3-keto-pyrrolidinyl, 2-keto-piperidinyl, 3-keto-piperidinyl, and 4-keto-piperidinyl);
a substituted or unsubstituted cyclic C₃-C₈ alkyl group (such as cyclopropyl, cyclobutyl, cyclopentyl, cyclohexyl, cycloheptyl and cyclooctyl);
a substituted or unsubstituted linear or branched C₂-C₆ alcohol group (such as —CH₂CH₂OH, —CH(CH₃)CH₂OH, —C(CH₃)₂OH, —CH₂CH₂CH₂OH, —CH₂CH₂CH₂CH₂OH, —CH(CH₃)CH₂CH₂OH, —CH(CH₃)CH(CH₃)OH, —CH(CH₂CH₃)CH₂OH, —C(CH₃)₂CH₂OH, —CH₂CH₂CH₂CH₂CH₂OH, and —CH₂CH₂CH₂CH₂CH₂CH₂OH);
a substituted or unsubstituted linear or branched C₂-C₆ carboxylic acid group (such as —CH₂COOH, —CH₂CH₂COOH, —CH₂CH₂CH₂COOH, —CH₂CH₂CH₂CH₂COOH, and —CH₂CH₂CH₂CH₂CH₂COOH);
a substituted or unsubstituted linear or branched carbonyl group (such as —(CO)Me, —(CO)Et, —(CO)Pr, —(CO)-i_Pr, —(CO)-n-Bu, —(CO)-i-Bu, —(CO)-t-Bu, —(CO) Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a substituted or unsubstituted linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a substituted or unsubstituted linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a substituted or unsubstituted sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃), —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe;
a substituted or unsubstituted aromatic group (such as Ph-, 2-F-Ph-, 3-F-Ph-4-F-Ph-, 2-Cl-Ph-, 3-Cl-Ph-, 4-Cl-Ph-, 2-Br-Ph-, 3-Br-Ph-, 4-Br-Ph-, 2-1-Ph-, 3-1-Ph, 4-1-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a substituted or unsubstituted saturated or unsaturated, substituted or unsubstituted, heterocyclic group including an aromatic heterocyclic group and/or a non-aromatic heterocyclic group (such as pyrrole-2-yl, pyrrole-3-yl, pyrazole-3-yl, pyrazole-4-yl, pyrazole-5-yl, imidazole-2-yl, imidazole-4-yl, imidazole-5-yl, 1,2,3-triazole-4-yl, 1,2,3-triazole-5-yl, 1,2,4-triazole-3-yl, 1,2,4-triazole-5-yl, pyridin-2-yl, pyridin-3-yl, pyridin-4-yl, pyridazine-3-yl, pyridazine-4-yl, pyrimidin-2-yl, pyrimidin-4-yl, pyrimidin-5-yl, pyrimidin-6-yl, pyrazine-2-yl, pyrrolidine-2-yl, pyrrolidine-3-yl, piperidine-2-yl, piperidine-3-yl, piperidine-4-yl, 2-azapiperidine-3-yl, 2-azapiperidine-4-yl, 3-azapiperidine-2-yl, 3-azapiperidine-4-yl, 3-azapiperidine-5-yl, piperazine-2-yl, furan-2-yl, furan-3-yl, pyran-2-yl, pyran-3-yl, pyran-4-yl, 2-azapyran-3-yl, 2-azapyran-4-yl, 2-azapyran-5-yl, 2-azapyran-6-yl, 3-azapyran-2-yl, 3-azapyran-4-yl, 3-azapyran-5-yl, 3-azapyran-6-yl, 4-azapyran-2-yl, 4-azapyran-3-yl, 4-azapyran-5-yl, 4-azapyran-6-yl, oxetan-3-yl, tetrahydrofuran-2-yl, tetrahydrofuran-3-yl, 2-aza-tetrahydrofuran-3-yl, 2-aza-tetrahydrofuran-4-yl, 2-aza-tetrahydrofuran-5-yl, 3-aza-tetrahydrofuran-2-yl, 3-aza-tetrahydrofuran-4-yl, 3-aza-tetrahydrofuran-5-yl, tetrahydropyran-2-yl, tetrahydropyran-3-yl, tetrahydropyran-4-yl, 2-aza-tetrahydropyran-3-yl, 2-aza-tetrahydropyran-4-yl, 2-aza-tetrahydropyran-5-yl, 2-aza-tetrahydropyran-6-yl, 3-aza-tetrahydropyran-2-yl, 3-aza-tetrahydropyran-4-yl, 3-aza-tetrahydropyran-5-yl, 3-aza-tetrahydropyran-6-yl, morpholine-2-yl, morpholine-3-yl, thiophen-2-yl, thiophen-3-yl, isothiazole-3-yl, isothiazole-4-yl, isothiazole-5-yl, thiazole-2-yl, thiazole-4-yl, thiazole-5-yl, thiopyran-2-yl, thiopyran-3-yl, thiopyran-4-yl, 2-azathiopyran-3-yl, 2-azathiopyran-4-yl, 2-azathiopyran-5-yl, 2-azathiopyran-6-yl, 3-azathiopyran-2-yl, 3-azathiopyran-4-yl, 3-azathiopyran-5-yl, 3-azathiopyran-6-yl, 4-azathiopyran-2-yl, 4-azathiopyran-3-yl, 4-azathiopyran-5-yl, 4-azathiopyran-6-yl, thiolane-2-yl, thiolane-3-yl, thiane-2-yl, thiane-3-yl, thiane-4-yl, oxazol-2-yl, oxazol-4-yl, oxazol-5-yl, isoxazol-3-yl, isoxazol-4-yl, isoxazol-5-yl, furazan-3-yl, (1,3,4-oxadiazol)-2-yl, (1,3,4-oxadiazol)-5-yl, (1,2,4-oxadiazol)-3-yl, (1,2,4-oxadiazol)-5-yl; and tetrazole-5-yl).

27. A compound according to claim 26, wherein R³, R⁶ and R⁹ are each independently selected from H, a substituted or unsubstituted C₁-C₆ alkyl group or a substituted or unsubstituted linear or branched C₁-C₆ halogenated alkyl group.

28. A compound according to claim 1, wherein R¹⁶ is absent or selected from H, a C₁-C₃ alkyl group and a C₁-C₃ halogenated alkyl group.

29. A compound according to claim 28, wherein R¹⁶ is H.

30. A compound according to claim 1, which is selected from:

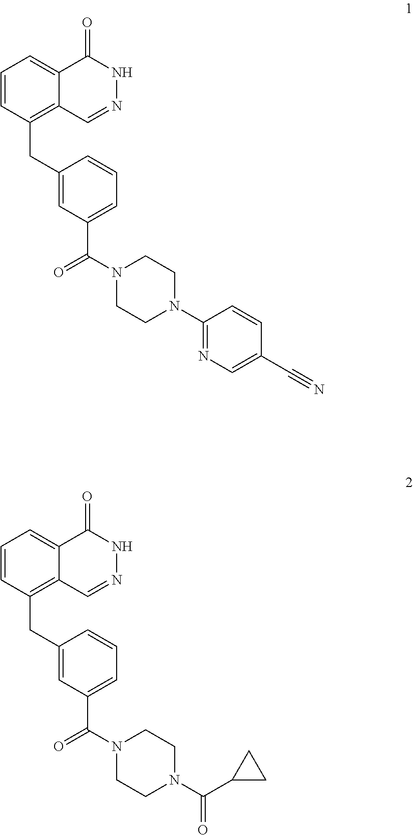 [SMILES: N#Cc1ccc(N2CCN(C(=O)c3cccc(Cc4cccc5c(=O)[nH]ncc45)c3)CC2)nc1 ; O=C(c1cccc(Cc2cccc3c(=O)[nH]ncc23)c1)N1CCN(C(=O)C2CC2)CC1]

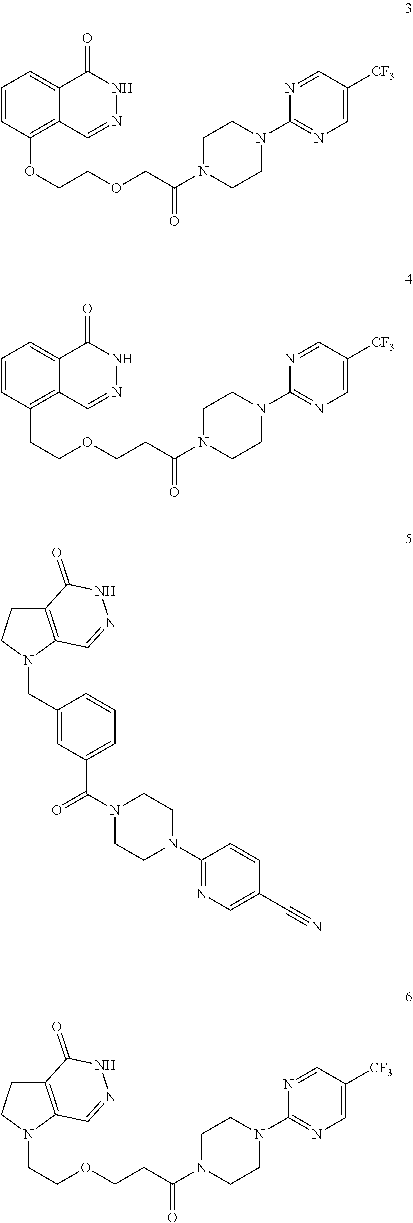 [SMILES: O=C(COCCOc1cccc2c(=O)[nH]ncc12)N1CCN(c2ncc(C(F)(F)F)cn2)CC1 ; O=C(CCOCCc1cccc2c(=O)[nH]ncc12)N1CCN(c2ncc(C(F)(F)F)cn2)CC1 ; N#Cc1ccc(N2CCN(C(=O)c3cccc(CN4CCc5c4cn[nH]c5=O)c3)CC2)nc1 ; O=C(CCOCCN1CCc2c1cn[nH]c2=O)N1CCN(c2ncc(C(F)(F)F)cn2)CC1]

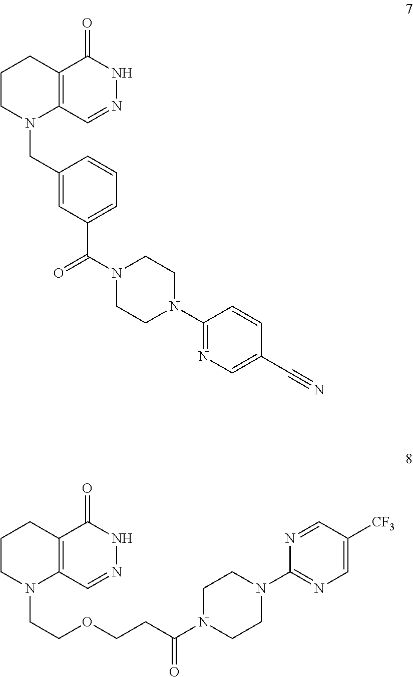 [SMILES: N#Cc1ccc(N2CCN(C(=O)c3cccc(CN4CCCc5c4cn[nH]c5=O)c3)CC2)nc1 ; O=C(CCOCCN1CCCc2c1cn[nH]c2=O)N1CCN(c2ncc(C(F)(F)F)cn2)CC1]

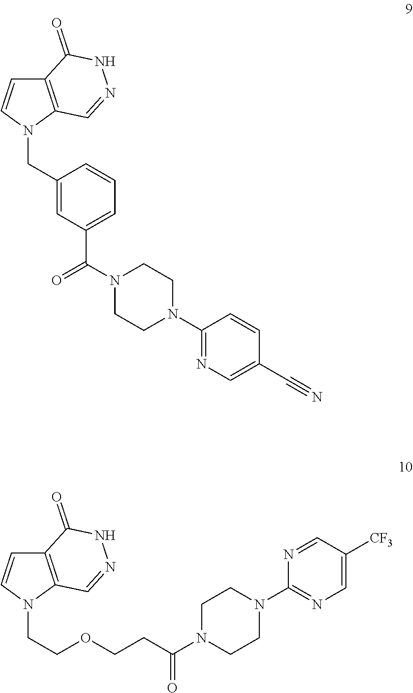 [SMILES: N#Cc1ccc(N2CCN(C(=O)c3cccc(Cn4ccc5c(=O)[nH]ncc54)c3)CC2)nc1 ; O=C(CCOCCn1ccc2c(=O)[nH]ncc21)N1CCN(c2ncc(C(F)(F)F)cn2)CC1]

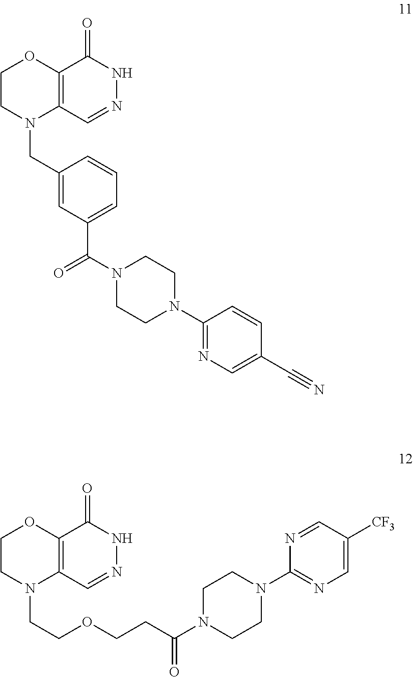 [SMILES: N#Cc1ccc(N2CCN(C(=O)c3cccc(CN4CCOc5c4cn[nH]c5=O)c3)CC2)nc1 ; O=C(CCOCCN1CCOc2c1cn[nH]c2=O)N1CCN(c2ncc(C(F)(F)F)cn2)CC1]

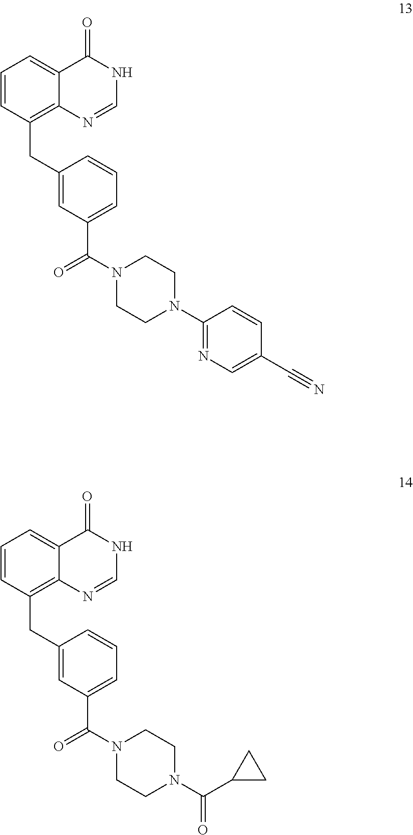 [SMILES: N#Cc1ccc(N2CCN(C(=O)c3cccc(Cc4cccc5c(=O)[nH]cnc45)c3)CC2)nc1 ; O=C(c1cccc(Cc2cccc3c(=O)[nH]cnc23)c1)N1CCN(C(=O)C2CC2)CC1]

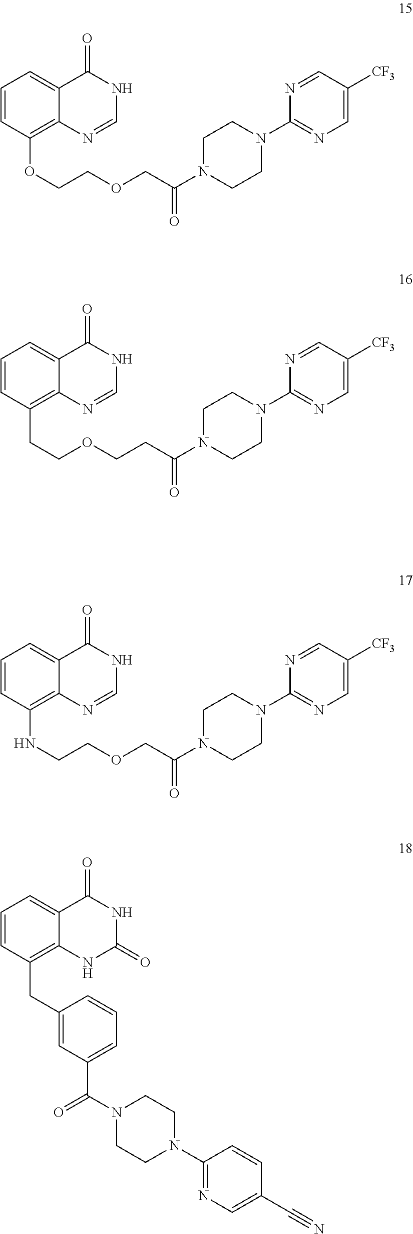 [SMILES: O=C(COCCOc1cccc2c(=O)[nH]cnc12)N1CCN(c2ncc(C(F)(F)F)cn2)CC1 ; O=C(CCOCCc1cccc2c(=O)[nH]cnc12)N1CCN(c2ncc(C(F)(F)F)cn2)CC1 ; O=C(COCCNc1cccc2c(=O)[nH]cnc12)N1CCN(c2ncc(C(F)(F)F)cn2)CC1 ; N#Cc1ccc(N2CCN(C(=O)c3cccc(Cc4cccc5c(=O)[nH]c(=O)[nH]c45)c3)CC2)nc1]

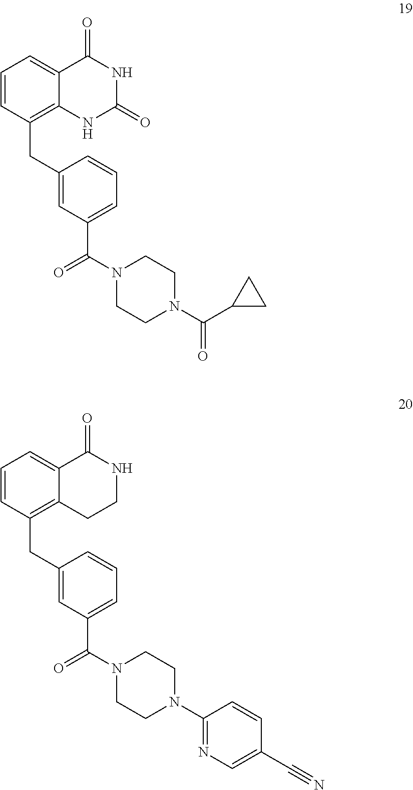 [SMILES: O=C(c1cccc(Cc2cccc3c(=O)[nH]c(=O)[nH]c23)c1)N1CCN(C(=O)C2CC2)CC1 ; N#Cc1ccc(N2CCN(C(=O)c3cccc(Cc4cccc5c4CCNC5=O)c3)CC2)nc1]

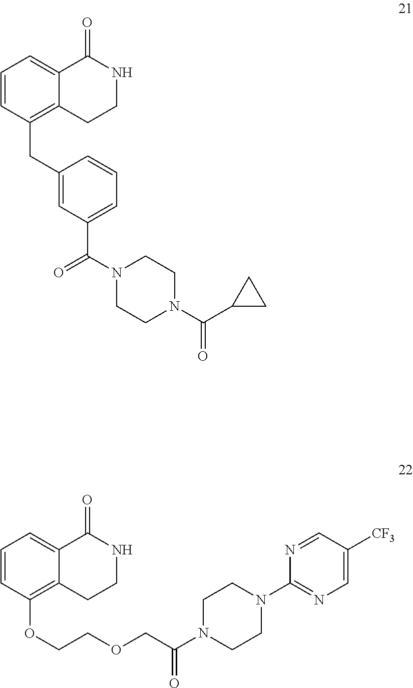 [SMILES: O=C1NCCc2c(Cc3cccc(C(=O)N4CCN(C(=O)C5CC5)CC4)c3)cccc21 ; O=C1NCCc2c(OCCOCC(=O)N3CCN(c4ncc(C(F)(F)F)cn4)CC3)cccc21]

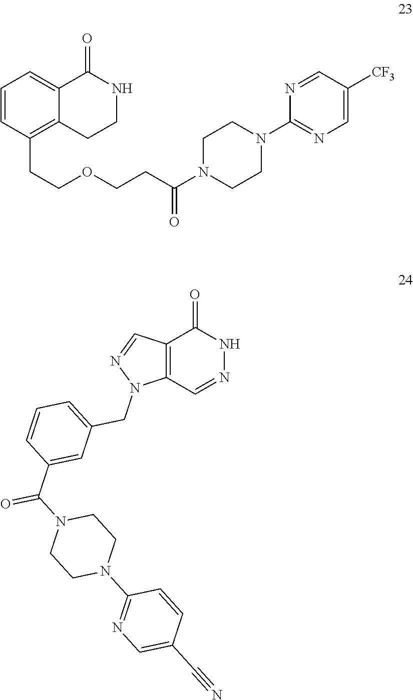 [SMILES: O=C1NCCc2c(CCOCCC(=O)N3CCN(c4ncc(C(F)(F)F)cn4)CC3)cccc21 ; N#Cc1ccc(N2CCN(C(=O)c3cccc(Cn4ncc5c(=O)[nH]ncc54)c3)CC2)nc1]

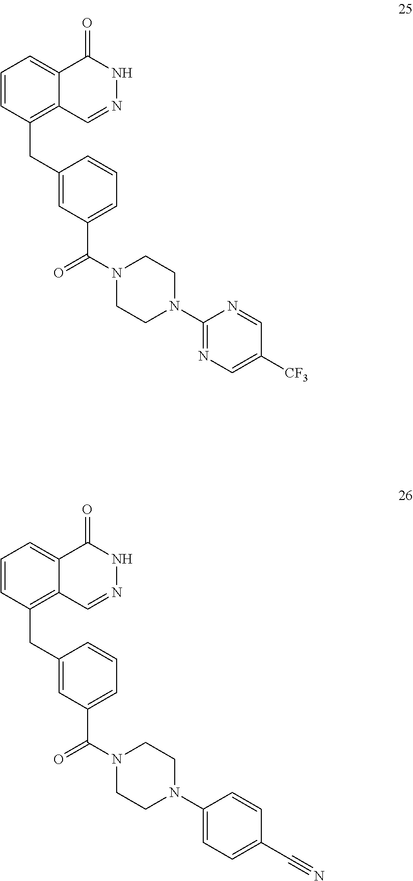 [SMILES: O=C(c1cccc(Cc2cccc3c(=O)[nH]ncc23)c1)N1CCN(c2ncc(C(F)(F)F)cn2)CC1 ; N#Cc1ccc(N2CCN(C(=O)c3cccc(Cc4cccc5c(=O)[nH]ncc45)c3)CC2)cc1]

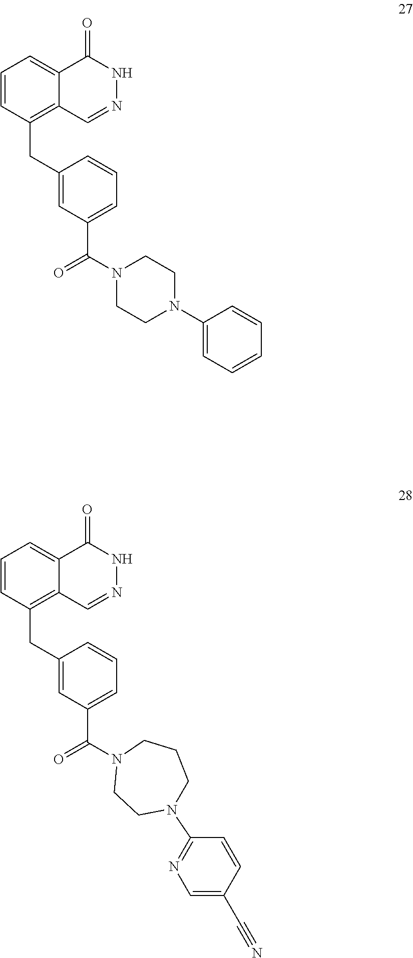 [SMILES: O=C(c1cccc(Cc2cccc3c(=O)[nH]ncc23)c1)N1CCN(c2ccccc2)CC1 ; N#Cc1ccc(N2CCCN(C(=O)c3cccc(Cc4cccc5c(=O)[nH]ncc45)c3)CC2)nc1]

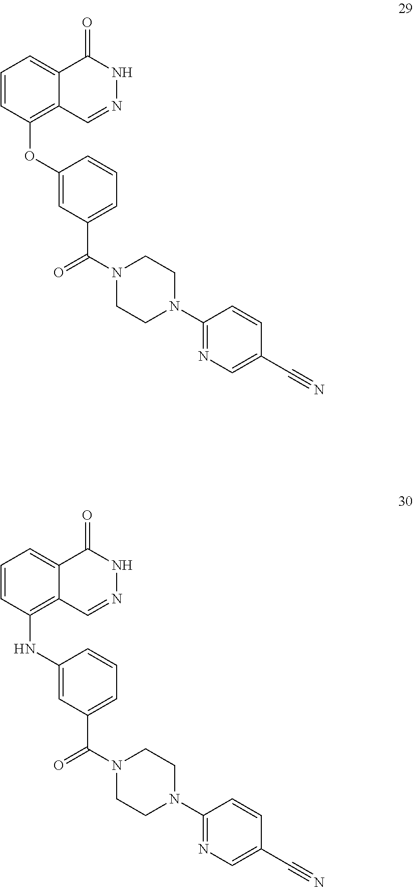 [SMILES: N#Cc1ccc(N2CCN(C(=O)c3cccc(Oc4cccc5c(=O)[nH]ncc45)c3)CC2)nc1 ; N#Cc1ccc(N2CCN(C(=O)c3cccc(Nc4cccc5c(=O)[nH]ncc45)c3)CC2)nc1]

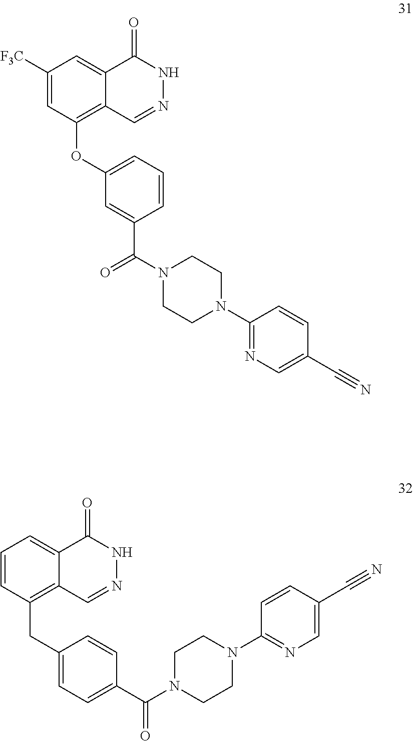 [SMILES: N#Cc1ccc(N2CCN(C(=O)c3cccc(Oc4cc(C(F)(F)F)cc5c(=O)[nH]ncc45)c3)CC2)nc1 ; N#Cc1ccc(N2CCN(C(=O)c3ccc(Cc4cccc5c(=O)[nH]ncc45)cc3)CC2)nc1]

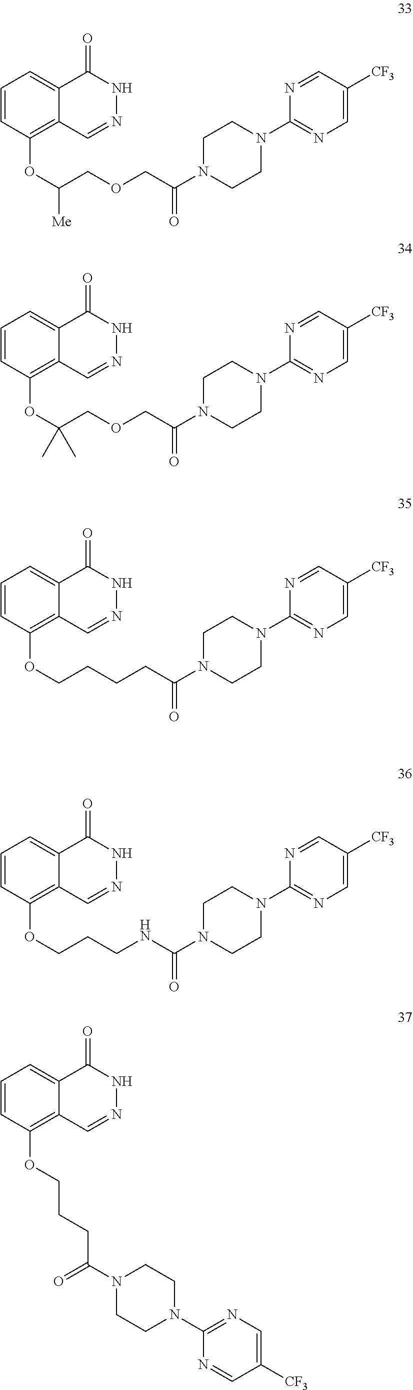 [SMILES: CC(COCC(=O)N1CCN(c2ncc(C(F)(F)F)cn2)CC1)Oc1cccc2c(=O)[nH]ncc12 ; CC(C)(COCC(=O)N1CCN(c2ncc(C(F)(F)F)cn2)CC1)Oc1cccc2c(=O)[nH]ncc12 ; O=C(CCCCOc1cccc2c(=O)[nH]ncc12)N1CCN(c2ncc(C(F)(F)F)cn2)CC1 ; O=C(NCCCOc1cccc2c(=O)[nH]ncc12)N1CCN(c2ncc(C(F)(F)F)cn2)CC1 ; O=C(CCCOc1cccc2c(=O)[nH]ncc12)N1CCN(c2ncc(C(F)(F)F)cn2)CC1]

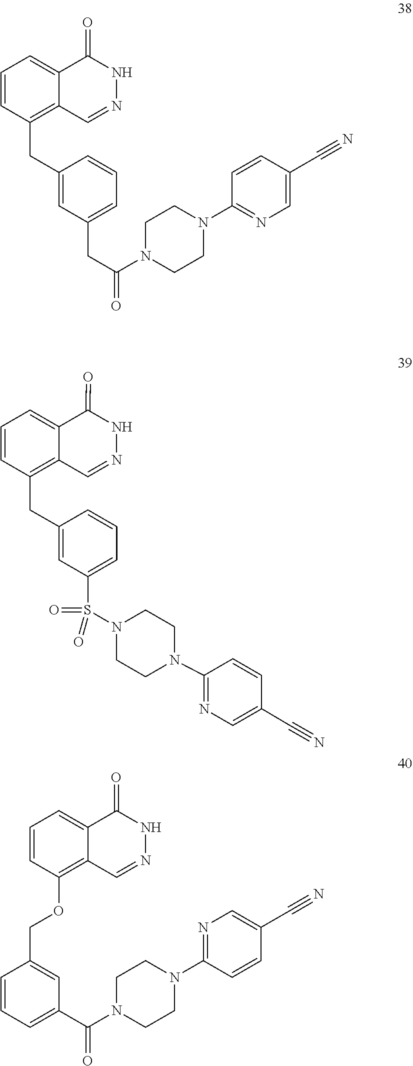 [SMILES: N#Cc1ccc(N2CCN(C(=O)Cc3cccc(Cc4cccc5c(=O)[nH]ncc45)c3)CC2)nc1 ; N#Cc1ccc(N2CCN(S(=O)(=O)c3cccc(Cc4cccc5c(=O)[nH]ncc45)c3)CC2)nc1 ; N#Cc1ccc(N2CCN(C(=O)c3cccc(COc4cccc5c(=O)[nH]ncc45)c3)CC2)nc1]

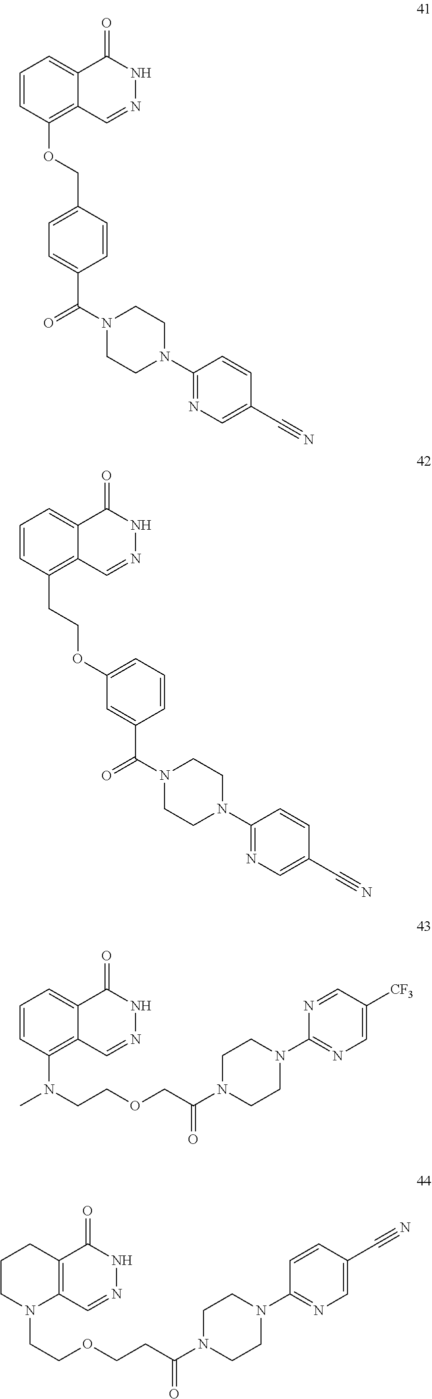 [SMILES: N#Cc1ccc(N2CCN(C(=O)c3ccc(COc4cccc5c(=O)[nH]ncc45)cc3)CC2)nc1 ; N#Cc1ccc(N2CCN(C(=O)c3cccc(OCCc4cccc5c(=O)[nH]ncc45)c3)CC2)nc1 ; CN(CCOCC(=O)N1CCN(c2ncc(C(F)(F)F)cn2)CC1)c1cccc2c(=O)[nH]ncc12 ; N#Cc1ccc(N2CCN(C(=O)CCOCCN3CCCc4c3cn[nH]c4=O)CC2)nc1]

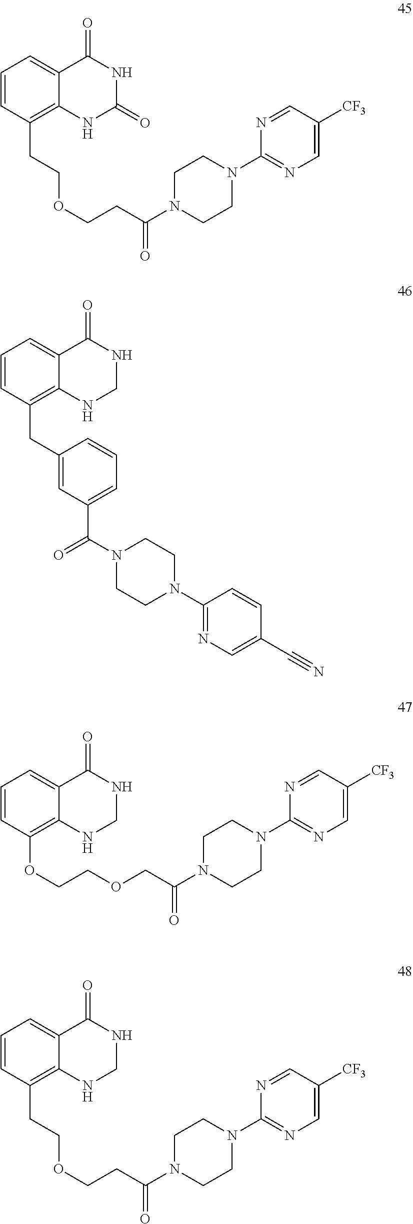 [SMILES: O=C(CCOCCc1cccc2c(=O)[nH]c(=O)[nH]c12)N1CCN(c2ncc(C(F)(F)F)cn2)CC1 ; N#Cc1ccc(N2CCN(C(=O)c3cccc(Cc4cccc5c4NCNC5=O)c3)CC2)nc1 ; O=C1NCNc2c(OCCOCC(=O)N3CCN(c4ncc(C(F)(F)F)cn4)CC3)cccc21 ; O=C1NCNc2c(CCOCCC(=O)N3CCN(c4ncc(C(F)(F)F)cn4)CC3)cccc21]

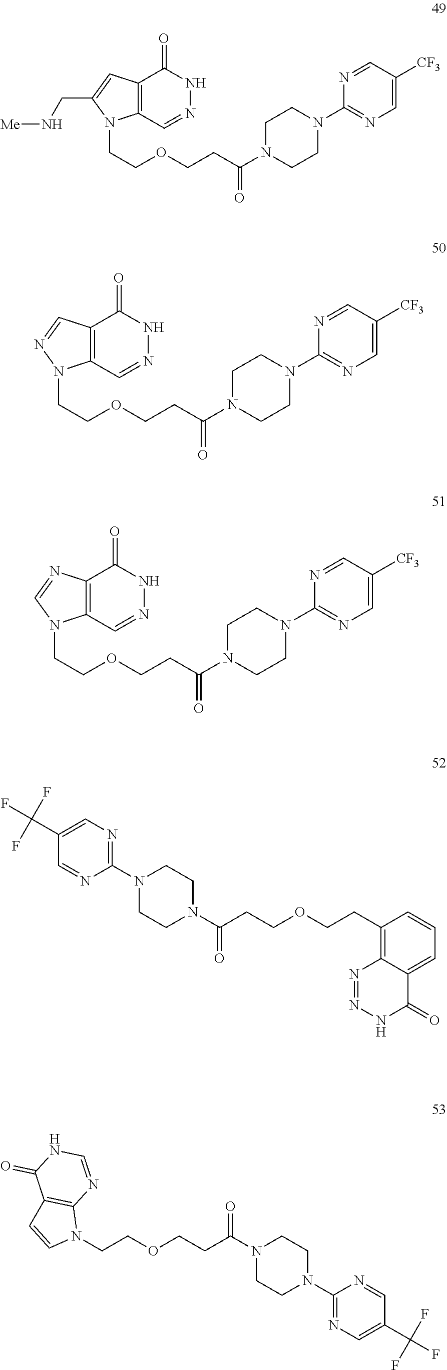 [SMILES: CNCc1cc2c(=O)[nH]ncc2n1CCOCCC(=O)N1CCN(c2ncc(C(F)(F)F)cn2)CC1 ; O=C(CCOCCn1ncc2c(=O)[nH]ncc21)N1CCN(c2ncc(C(F)(F)F)cn2)CC1 ; O=C(CCOCCn1cnc2c(=O)[nH]ncc21)N1CCN(c2ncc(C(F)(F)F)cn2)CC1 ; O=C(CCOCCc1cccc2c(=O)[nH]nnc12)N1CCN(c2ncc(C(F)(F)F)cn2)CC1 ; O=C(CCOCCn1ccc2c(=O)[nH]cnc21)N1CCN(c2ncc(C(F)(F)F)cn2)CC1]

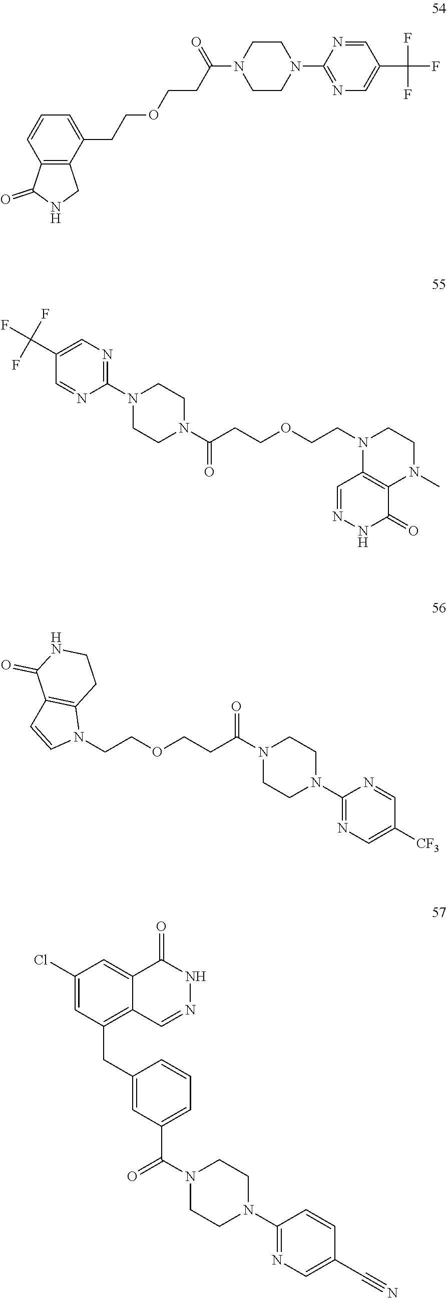 [SMILES: O=C1NCc2c(CCOCCC(=O)N3CCN(c4ncc(C(F)(F)F)cn4)CC3)cccc21 ; CN1CCN(CCOCCC(=O)N2CCN(c3ncc(C(F)(F)F)cn3)CC2)c2cn[nH]c(=O)c21 ; O=C1NCCc2c1ccn2CCOCCC(=O)N1CCN(c2ncc(C(F)(F)F)cn2)CC1 ; N#Cc1ccc(N2CCN(C(=O)c3cccc(Cc4cc(Cl)cc5c(=O)[nH]ncc45)c3)CC2)nc1]

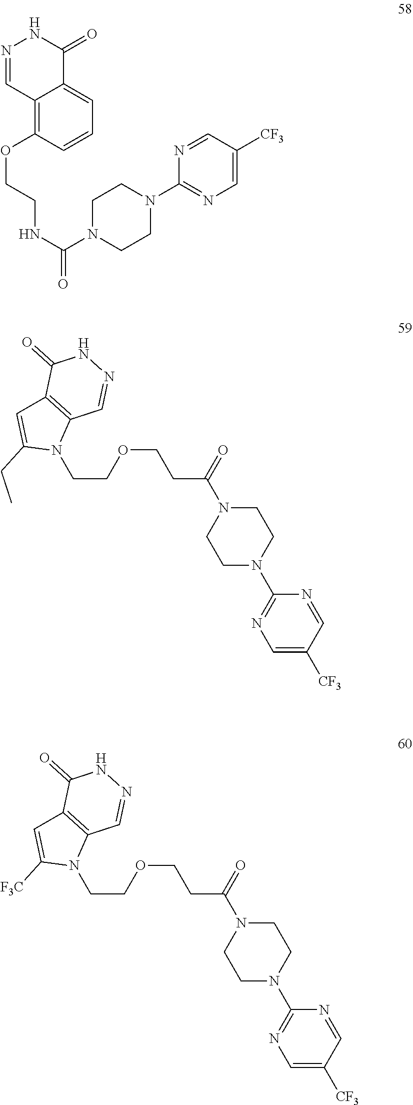 [SMILES: O=C(NCCOc1cccc2c(=O)[nH]ncc12)N1CCN(c2ncc(C(F)(F)F)cn2)CC1 ; CCc1cc2c(=O)[nH]ncc2n1CCOCCC(=O)N1CCN(c2ncc(C(F)(F)F)cn2)CC1 ; O=C(CCOCCn1c(C(F)(F)F)cc2c(=O)[nH]ncc21)N1CCN(c2ncc(C(F)(F)F)cn2)CC1]

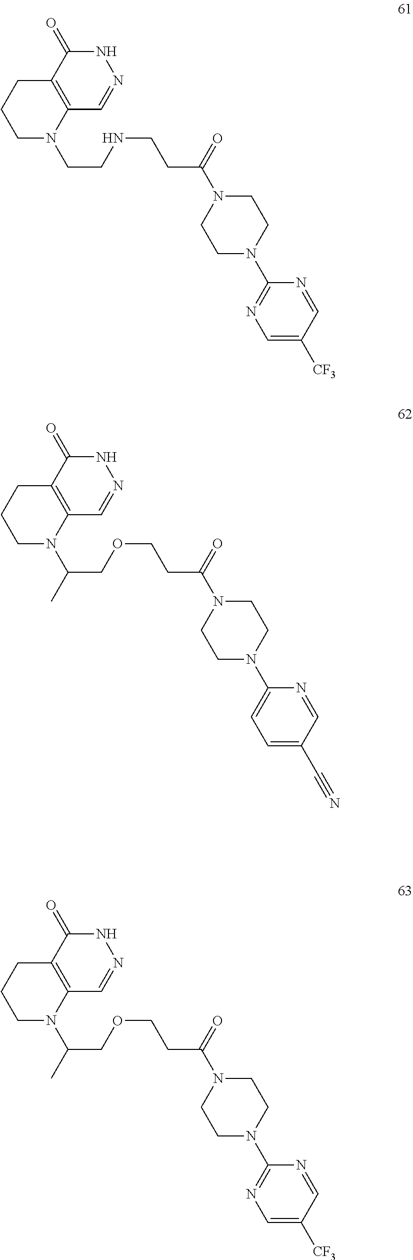 [SMILES: O=C(CCNCCN1CCCc2c1cn[nH]c2=O)N1CCN(c2ncc(C(F)(F)F)cn2)CC1 ; CC(COCCC(=O)N1CCN(c2ccc(C#N)cn2)CC1)N1CCCc2c1cn[nH]c2=O ; CC(COCCC(=O)N1CCN(c2ncc(C(F)(F)F)cn2)CC1)N1CCCc2c1cn[nH]c2=O]

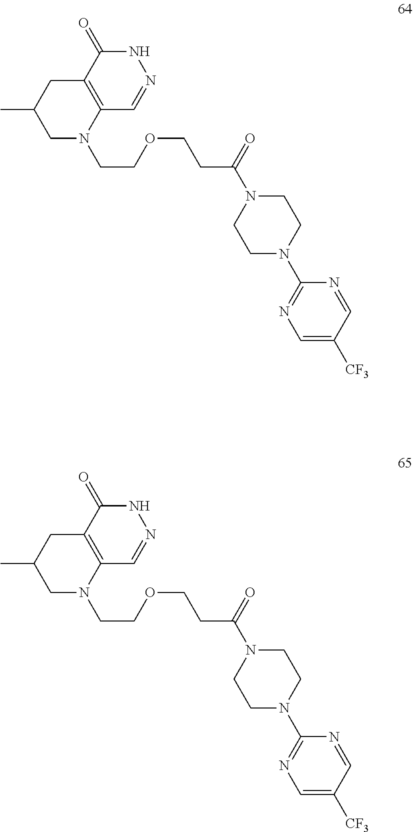 [SMILES: CC1Cc2c(cn[nH]c2=O)N(CCOCCC(=O)N2CCN(c3ncc(C(F)(F)F)cn3)CC2)C1 ; CC1Cc2c(cn[nH]c2=O)N(CCOCCC(=O)N2CCN(c3ncc(C(F)(F)F)cn3)CC2)C1]

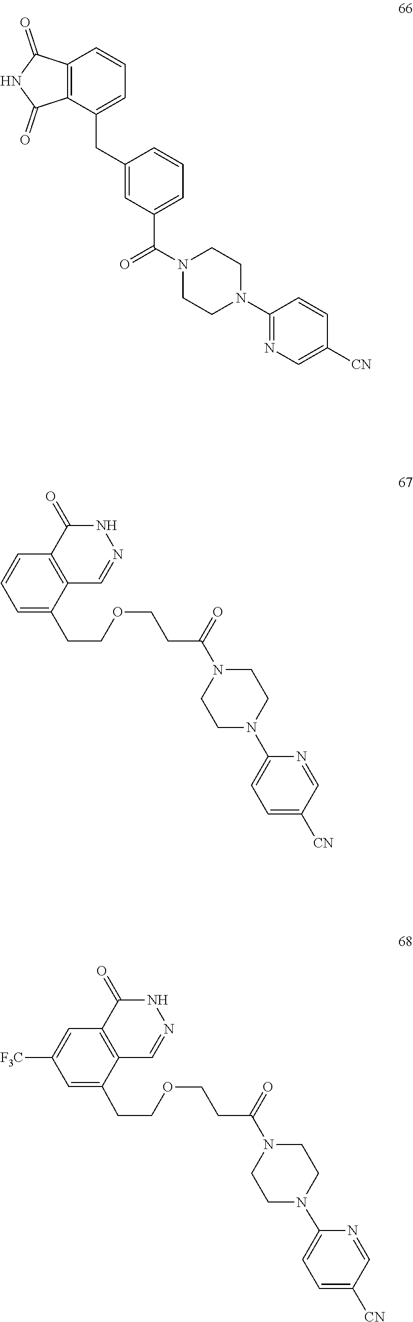 [SMILES: N#Cc1ccc(N2CCN(C(=O)c3cccc(Cc4cccc5c4C(=O)NC5=O)c3)CC2)nc1 ; N#Cc1ccc(N2CCN(C(=O)CCOCCc3cccc4c(=O)[nH]ncc34)CC2)nc1 ; N#Cc1ccc(N2CCN(C(=O)CCOCCc3cc(C(F)(F)F)cc4c(=O)[nH]ncc34)CC2)nc1]

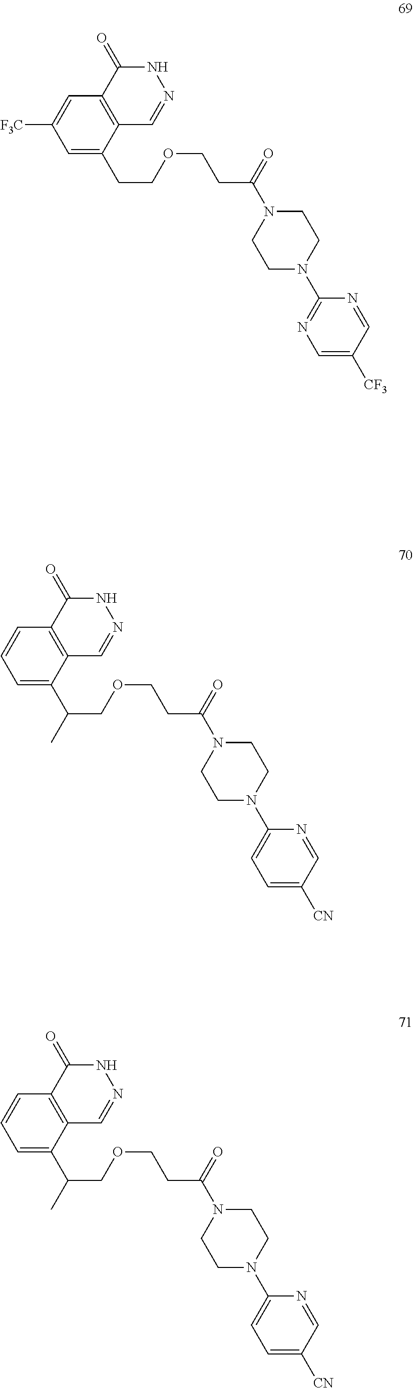 [SMILES: O=C(CCOCCc1cc(C(F)(F)F)cc2c(=O)[nH]ncc12)N1CCN(c2ncc(C(F)(F)F)cn2)CC1 ; CC(COCCC(=O)N1CCN(c2ccc(C#N)cn2)CC1)c1cccc2c(=O)[nH]ncc12 ; CC(COCCC(=O)N1CCN(c2ccc(C#N)cn2)CC1)c1cccc2c(=O)[nH]ncc12]

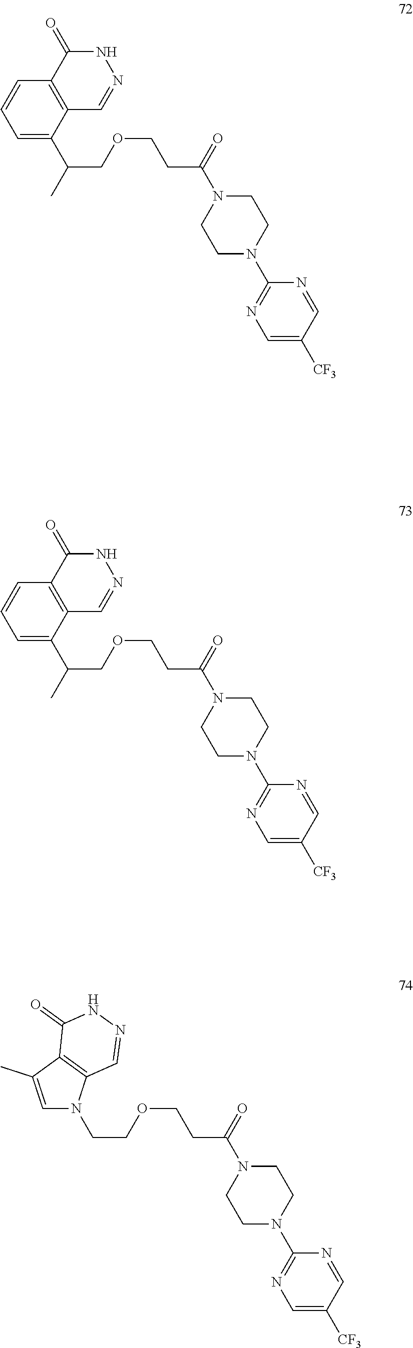 [SMILES: CC(COCCC(=O)N1CCN(c2ncc(C(F)(F)F)cn2)CC1)c1cccc2c(=O)[nH]ncc12 ; CC(COCCC(=O)N1CCN(c2ncc(C(F)(F)F)cn2)CC1)c1cccc2c(=O)[nH]ncc12 ; Cc1cn(CCOCCC(=O)N2CCN(c3ncc(C(F)(F)F)cn3)CC2)c2cn[nH]c(=O)c12]

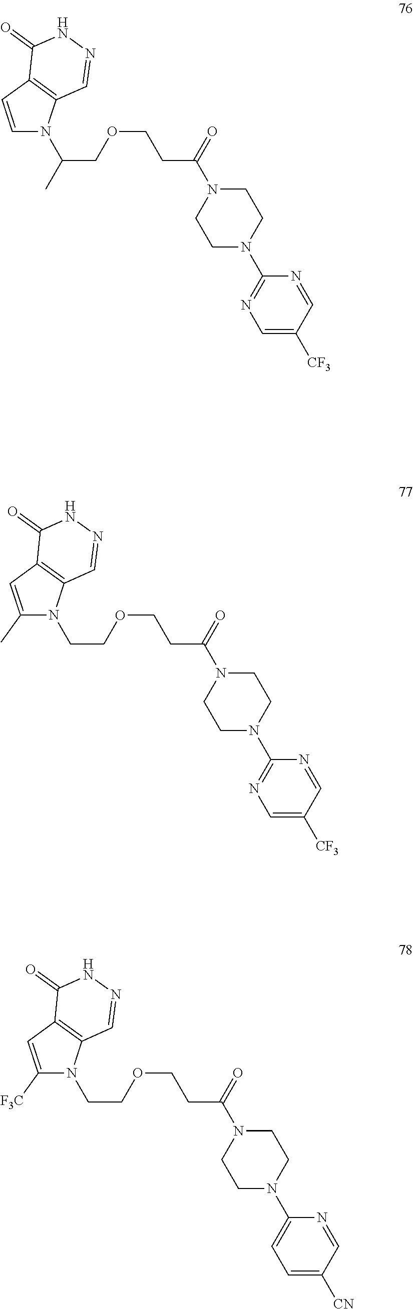 [SMILES: CC(COCCC(=O)N1CCN(c2ncc(C(F)(F)F)cn2)CC1)n1ccc2c(=O)[nH]ncc21 ; Cc1cc2c(=O)[nH]ncc2n1CCOCCC(=O)N1CCN(c2ncc(C(F)(F)F)cn2)CC1 ; N#Cc1ccc(N2CCN(C(=O)CCOCCn3c(C(F)(F)F)cc4c(=O)[nH]ncc43)CC2)nc1]

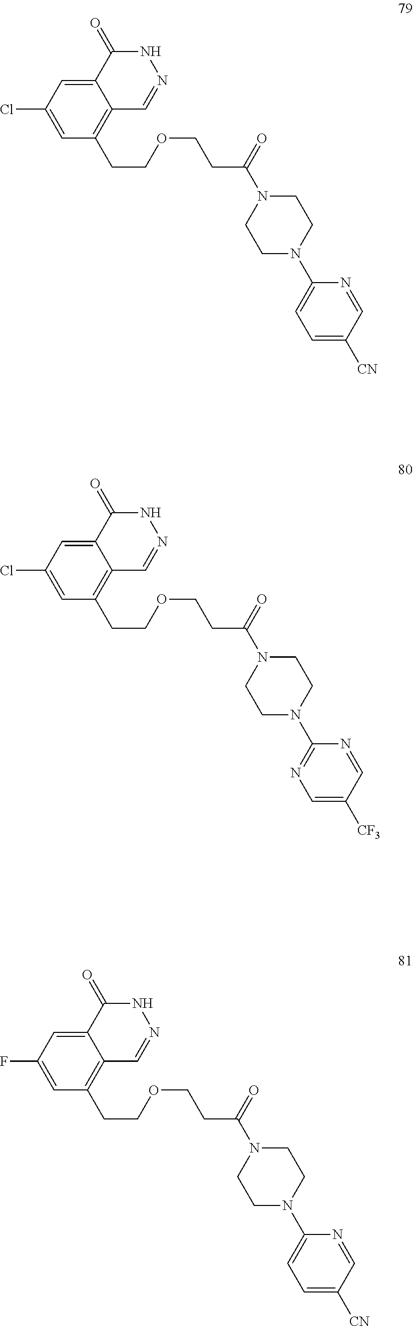 [SMILES: N#Cc1ccc(N2CCN(C(=O)CCOCCc3cc(Cl)cc4c(=O)[nH]ncc34)CC2)nc1 ; O=C(CCOCCc1cc(Cl)cc2c(=O)[nH]ncc12)N1CCN(c2ncc(C(F)(F)F)cn2)CC1 ; N#Cc1ccc(N2CCN(C(=O)CCOCCc3cc(F)cc4c(=O)[nH]ncc34)CC2)nc1]

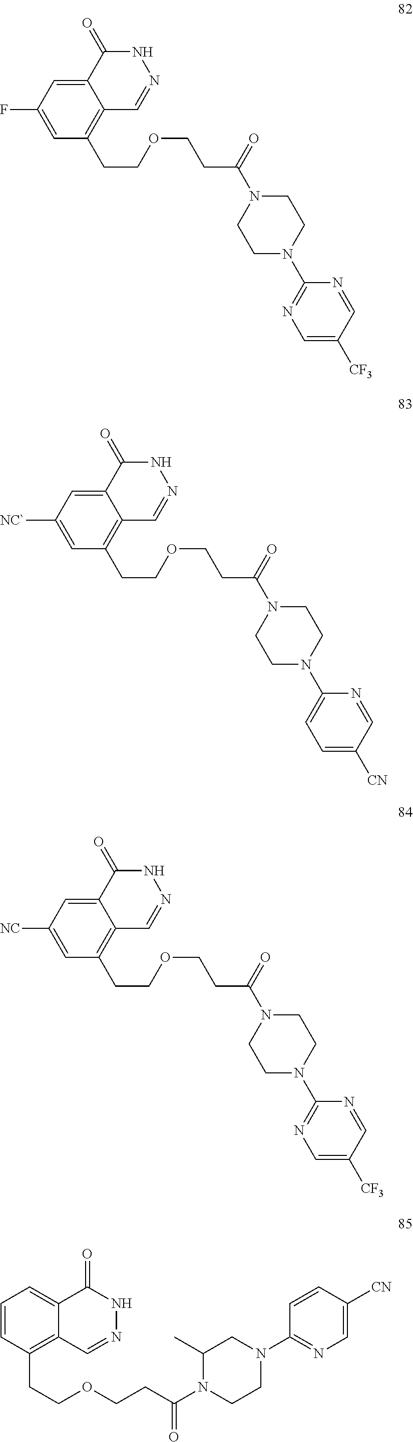

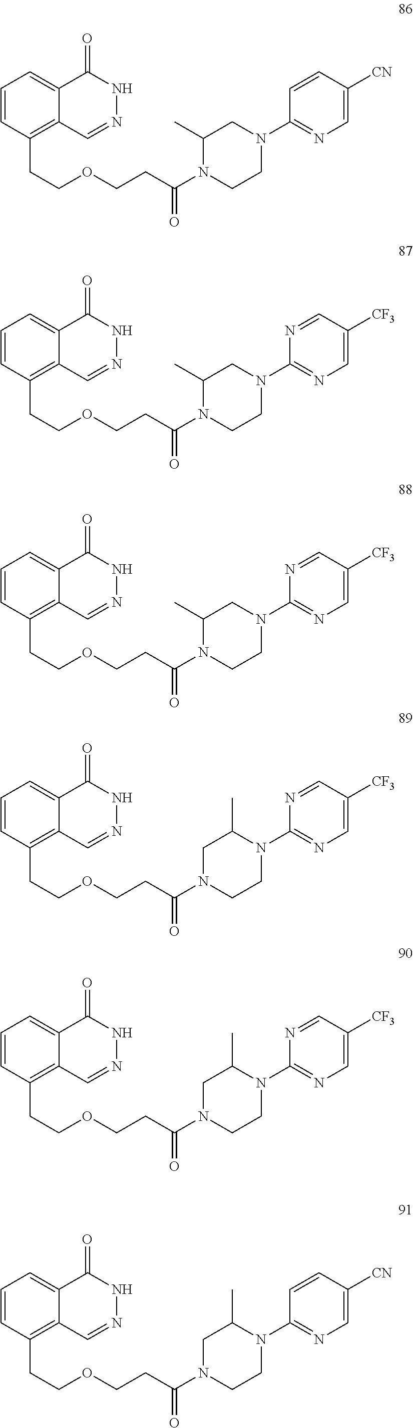 [SMILES: CC1CN(c2ccc(C#N)cn2)CCN1C(=O)CCOCCc1cccc2c(=O)[nH]ncc12 ; CC1CN(c2ncc(C(F)(F)F)cn2)CCN1C(=O)CCOCCc1cccc2c(=O)[nH]ncc12 ; CC1CN(c2ncc(C(F)(F)F)cn2)CCN1C(=O)CCOCCc1cccc2c(=O)[nH]ncc12 ; CC1CN(C(=O)CCOCCc2cccc3c(=O)[nH]ncc23)CCN1c1ncc(C(F)(F)F)cn1 ; CC1CN(C(=O)CCOCCc2cccc3c(=O)[nH]ncc23)CCN1c1ncc(C(F)(F)F)cn1 ; CC1CN(C(=O)CCOCCc2cccc3c(=O)[nH]ncc23)CCN1c1ccc(C#N)cn1]

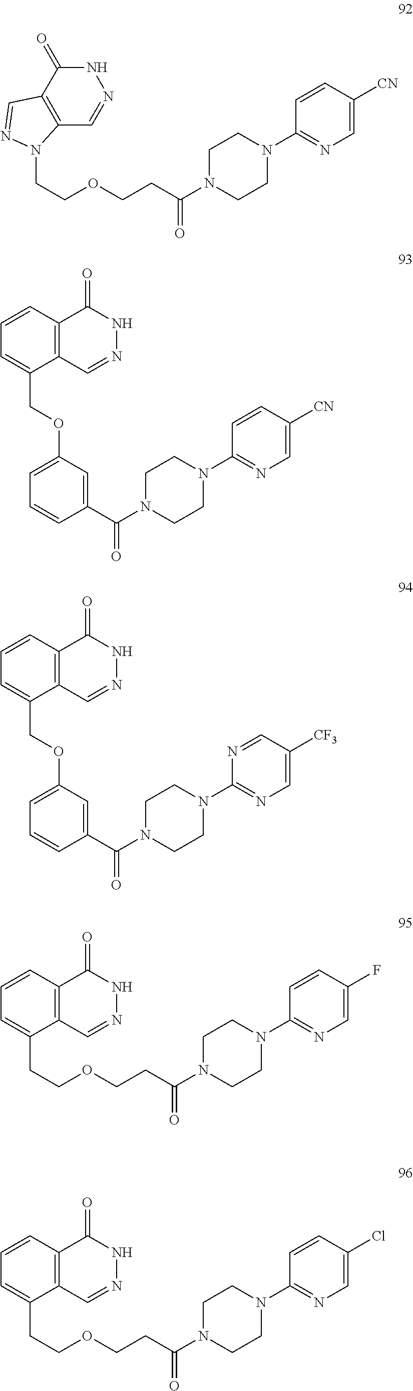 [SMILES: N#Cc1ccc(N2CCN(C(=O)CCOCCn3ncc4c(=O)[nH]ncc43)CC2)nc1 ; N#Cc1ccc(N2CCN(C(=O)c3cccc(OCc4cccc5c(=O)[nH]ncc45)c3)CC2)nc1 ; O=C(c1cccc(OCc2cccc3c(=O)[nH]ncc23)c1)N1CCN(c2ncc(C(F)(F)F)cn2)CC1 ; O=C(CCOCCc1cccc2c(=O)[nH]ncc12)N1CCN(c2ccc(F)cn2)CC1 ; O=C(CCOCCc1cccc2c(=O)[nH]ncc12)N1CCN(c2ccc(Cl)cn2)CC1]

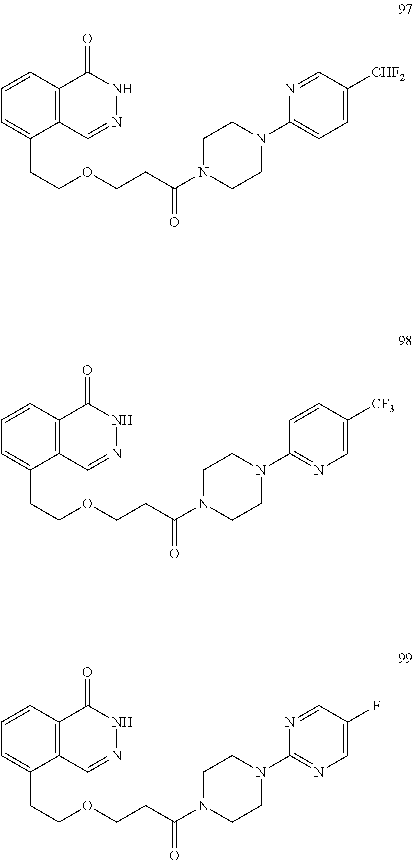 [SMILES: O=C(CCOCCc1cccc2c(=O)[nH]ncc12)N1CCN(c2ccc(C(F)F)cn2)CC1 ; O=C(CCOCCc1cccc2c(=O)[nH]ncc12)N1CCN(c2ccc(C(F)(F)F)cn2)CC1 ; O=C(CCOCCc1cccc2c(=O)[nH]ncc12)N1CCN(c2ncc(F)cn2)CC1]

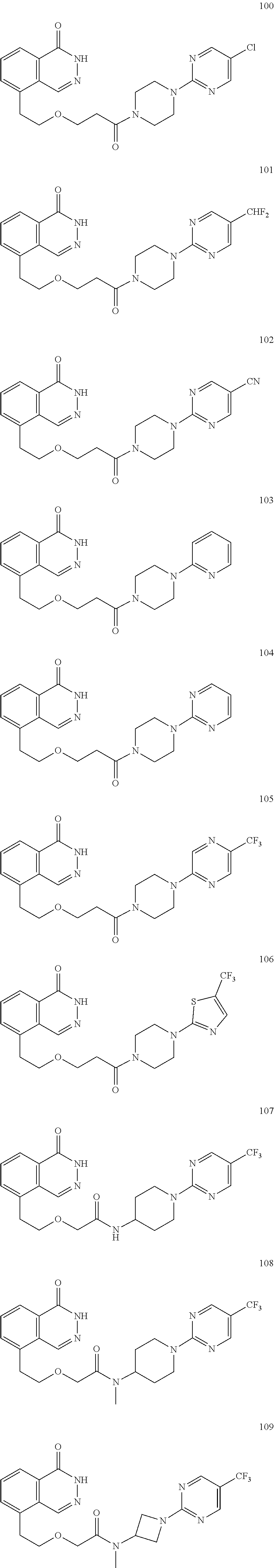

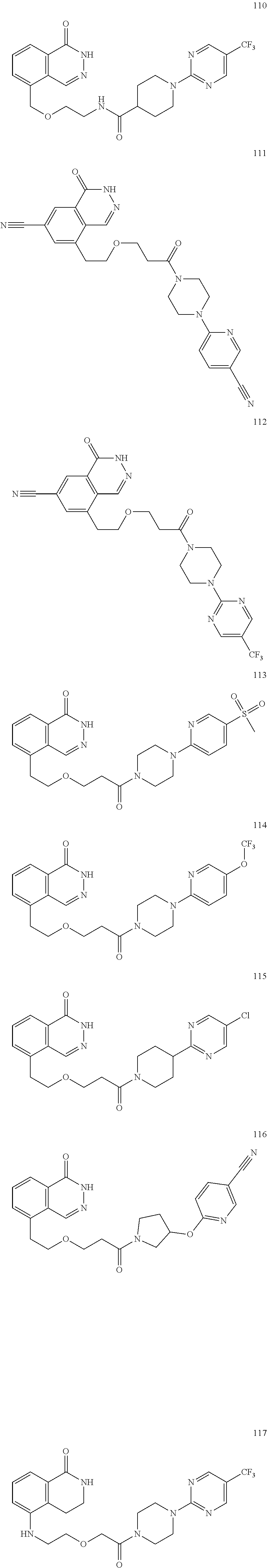

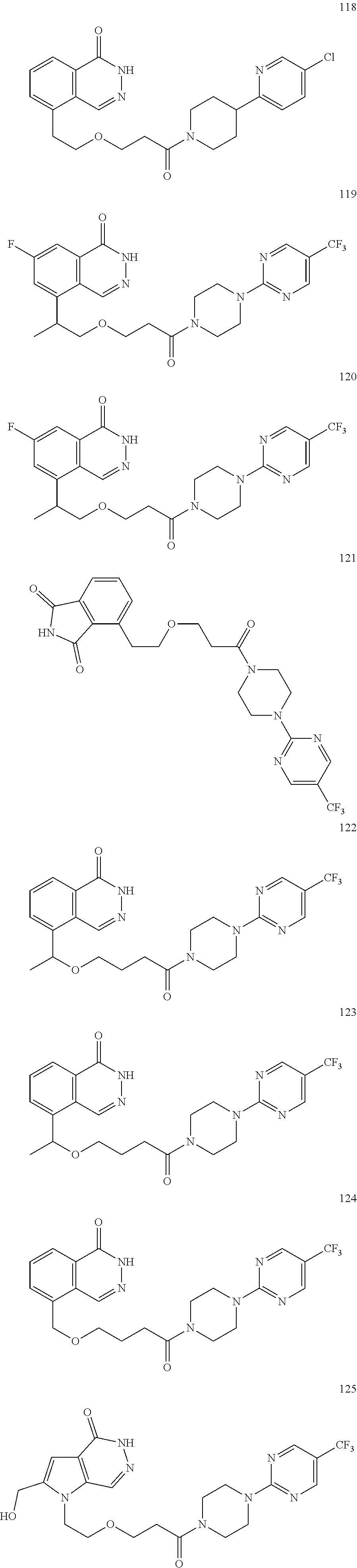

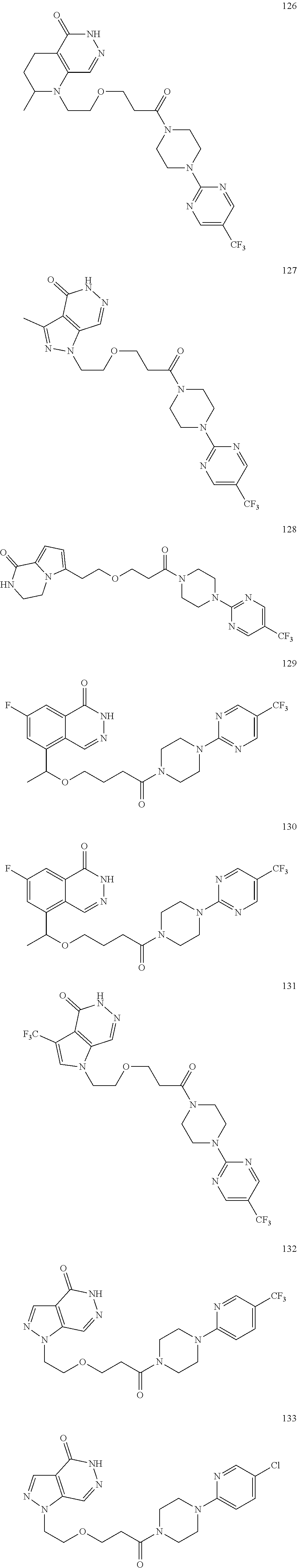

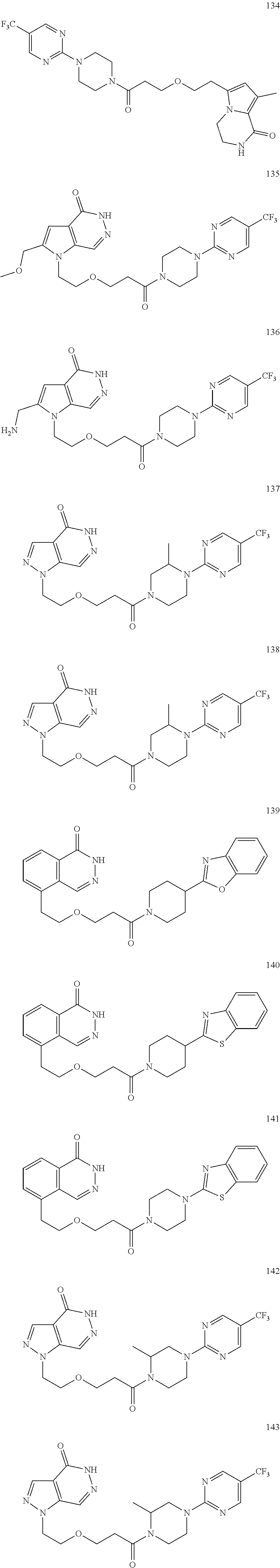

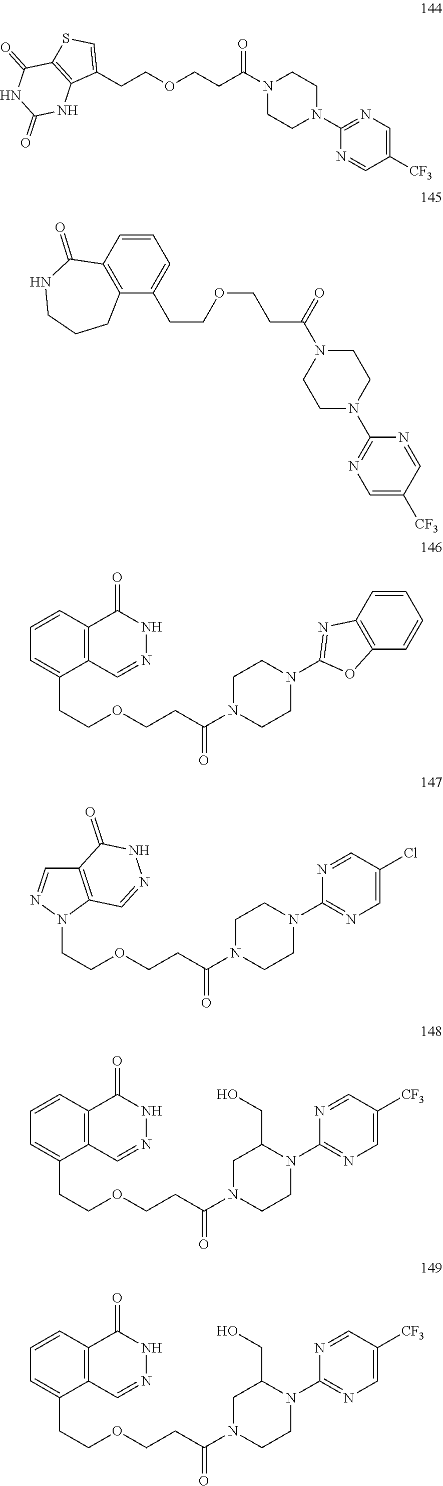 [SMILES: O=C(CCOCCc1csc2c(=O)[nH]c(=O)[nH]c12)N1CCN(c2ncc(C(F)(F)F)cn2)CC1 ; O=C1NCCCc2c(CCOCCC(=O)N3CCN(c4ncc(C(F)(F)F)cn4)CC3)cccc21 ; O=C(CCOCCc1cccc2c(=O)[nH]ncc12)N1CCN(c2nc3ccccc3o2)CC1 ; O=C(CCOCCn1ncc2c(=O)[nH]ncc21)N1CCN(c2ncc(Cl)cn2)CC1 ; O=C(CCOCCc1cccc2c(=O)[nH]ncc12)N1CCN(c2ncc(C(F)(F)F)cn2)C(CO)C1 ; O=C(CCOCCc1cccc2c(=O)[nH]ncc12)N1CCN(c2ncc(C(F)(F)F)cn2)C(CO)C1]

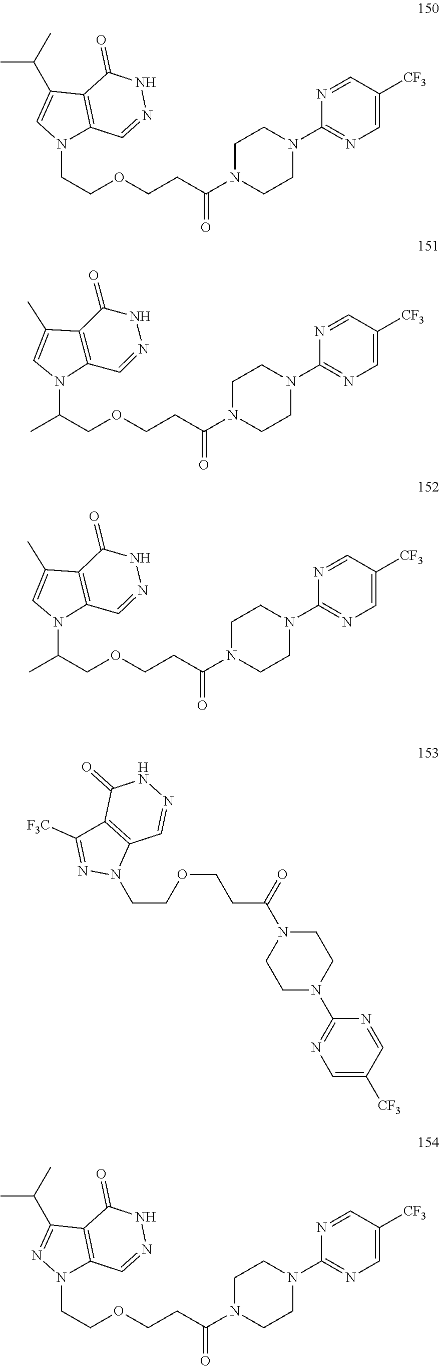 [SMILES: CC(C)c1cn(CCOCCC(=O)N2CCN(c3ncc(C(F)(F)F)cn3)CC2)c2cn[nH]c(=O)c12 ; Cc1cn(C(C)COCCC(=O)N2CCN(c3ncc(C(F)(F)F)cn3)CC2)c2cn[nH]c(=O)c12 ; Cc1cn(C(C)COCCC(=O)N2CCN(c3ncc(C(F)(F)F)cn3)CC2)c2cn[nH]c(=O)c12 ; O=C(CCOCCn1nc(C(F)(F)F)c2c(=O)[nH]ncc21)N1CCN(c2ncc(C(F)(F)F)cn2)CC1 ; CC(C)c1nn(CCOCCC(=O)N2CCN(c3ncc(C(F)(F)F)cn3)CC2)c2cn[nH]c(=O)c12]

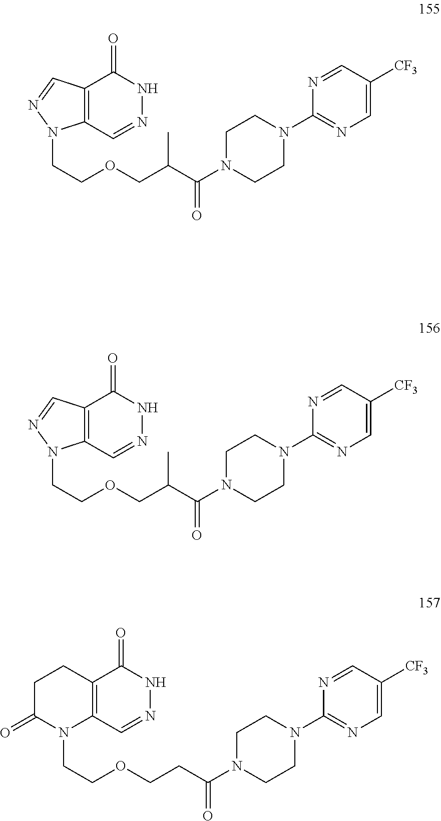 [SMILES: CC(COCCn1ncc2c(=O)[nH]ncc21)C(=O)N1CCN(c2ncc(C(F)(F)F)cn2)CC1 ; CC(COCCn1ncc2c(=O)[nH]ncc21)C(=O)N1CCN(c2ncc(C(F)(F)F)cn2)CC1 ; O=C(CCOCCN1C(=O)CCc2c1cn[nH]c2=O)N1CCN(c2ncc(C(F)(F)F)cn2)CC1]

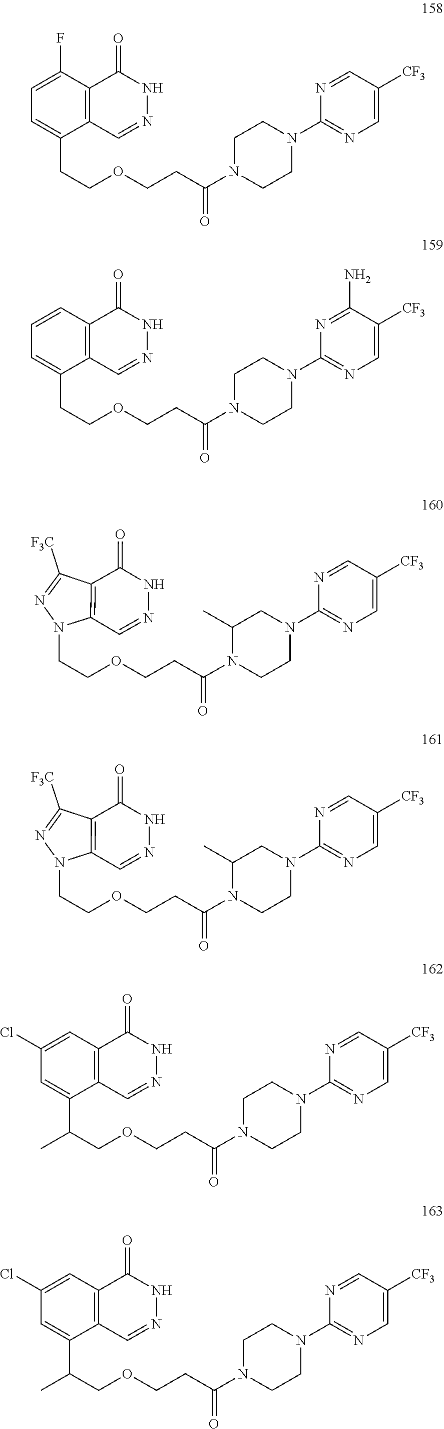 [SMILES: O=C(CCOCCc1ccc(F)c2c(=O)[nH]ncc12)N1CCN(c2ncc(C(F)(F)F)cn2)CC1 ; Nc1nc(N2CCN(C(=O)CCOCCc3cccc4c(=O)[nH]ncc34)CC2)ncc1C(F)(F)F ; CC1CN(c2ncc(C(F)(F)F)cn2)CCN1C(=O)CCOCCn1nc(C(F)(F)F)c2c(=O)[nH]ncc21 ; CC1CN(c2ncc(C(F)(F)F)cn2)CCN1C(=O)CCOCCn1nc(C(F)(F)F)c2c(=O)[nH]ncc21 ; CC(COCCC(=O)N1CCN(c2ncc(C(F)(F)F)cn2)CC1)c1cc(Cl)cc2c(=O)[nH]ncc12 ; CC(COCCC(=O)N1CCN(c2ncc(C(F)(F)F)cn2)CC1)c1cc(Cl)cc2c(=O)[nH]ncc12]

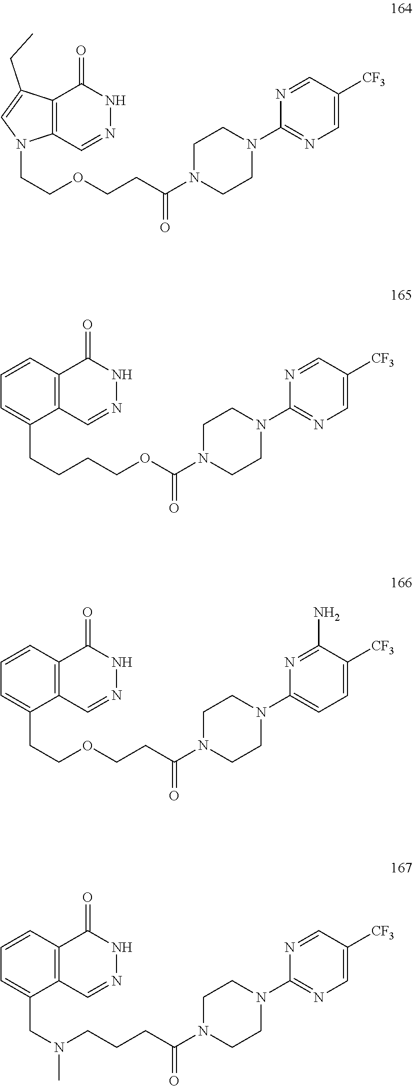 [SMILES: CCc1cn(CCOCCC(=O)N2CCN(c3ncc(C(F)(F)F)cn3)CC2)c2cn[nH]c(=O)c12 ; O=C(OCCCCc1cccc2c(=O)[nH]ncc12)N1CCN(c2ncc(C(F)(F)F)cn2)CC1 ; Nc1nc(N2CCN(C(=O)CCOCCc3cccc4c(=O)[nH]ncc34)CC2)ccc1C(F)(F)F ; CN(CCCC(=O)N1CCN(c2ncc(C(F)(F)F)cn2)CC1)Cc1cccc2c(=O)[nH]ncc12]

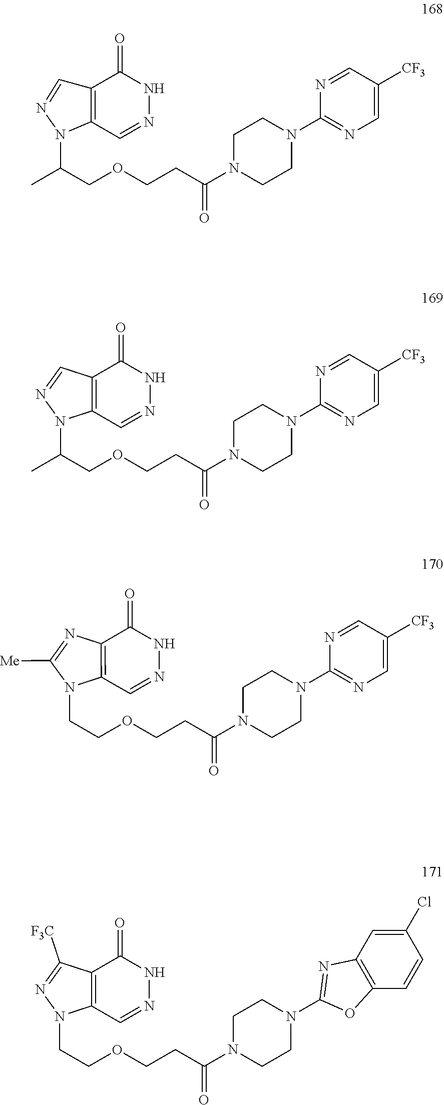

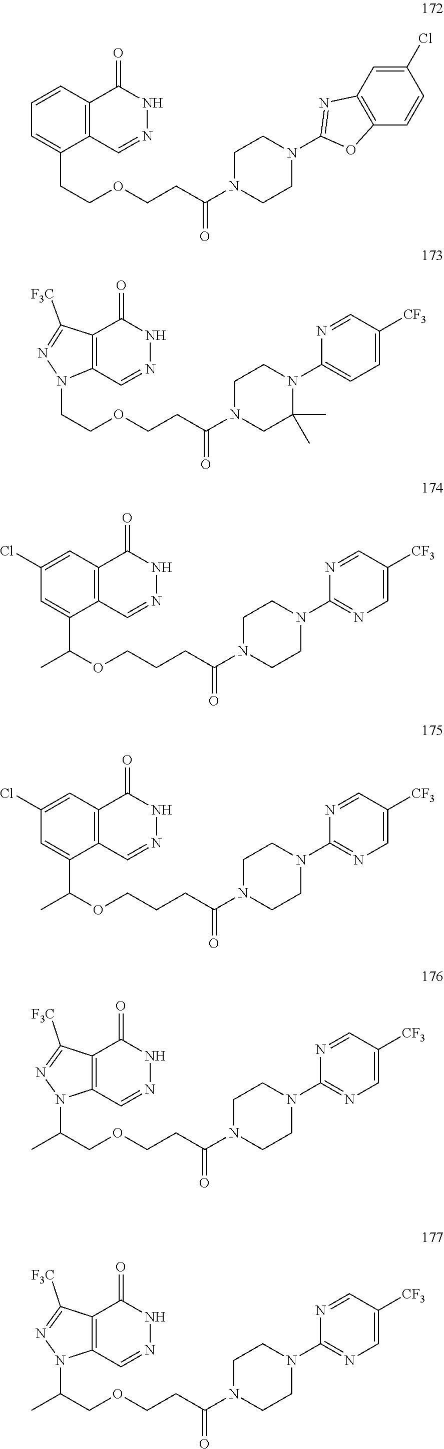

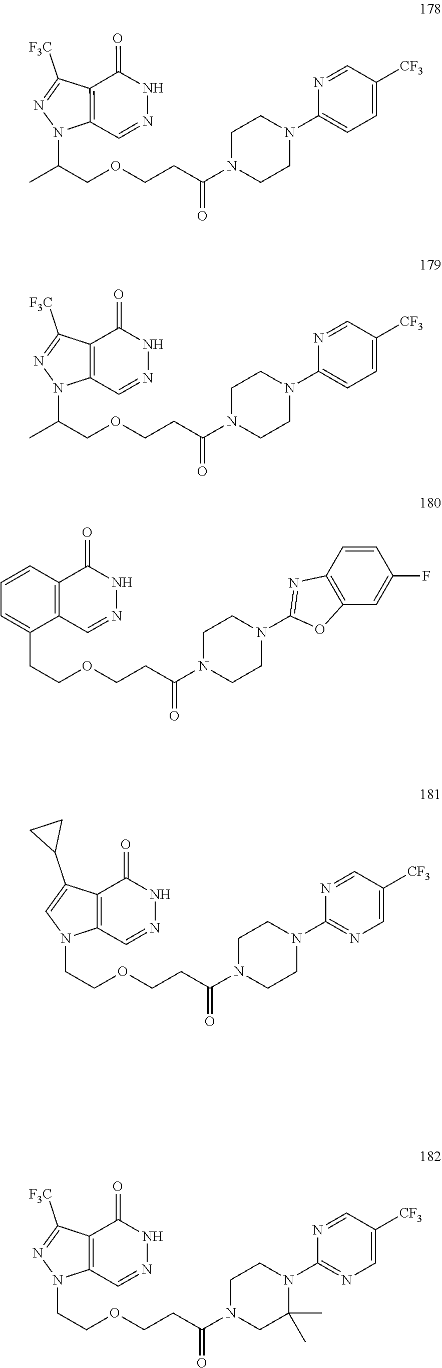 [SMILES: CC(COCCC(=O)N1CCN(c2ccc(C(F)(F)F)cn2)CC1)n1nc(C(F)(F)F)c2c(=O)[nH]ncc21 ; CC(COCCC(=O)N1CCN(c2ccc(C(F)(F)F)cn2)CC1)n1nc(C(F)(F)F)c2c(=O)[nH]ncc21 ; O=C(CCOCCc1cccc2c(=O)[nH]ncc12)N1CCN(c2nc3ccc(F)cc3o2)CC1 ; O=C(CCOCCn1cc(C2CC2)c2c(=O)[nH]ncc21)N1CCN(c2ncc(C(F)(F)F)cn2)CC1 ; CC1(C)CN(C(=O)CCOCCn2nc(C(F)(F)F)c3c(=O)[nH]ncc32)CCN1c1ncc(C(F)(F)F)cn1]

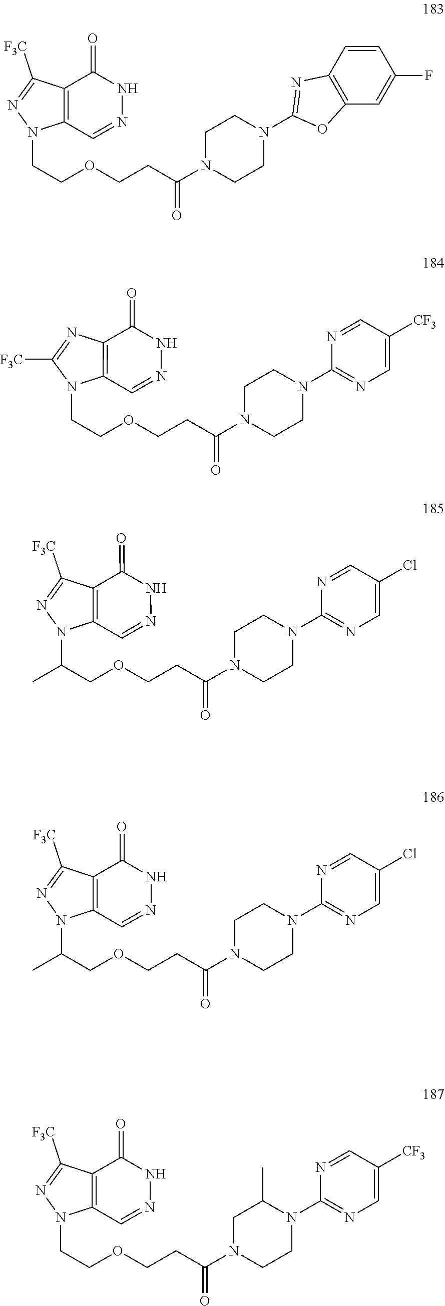

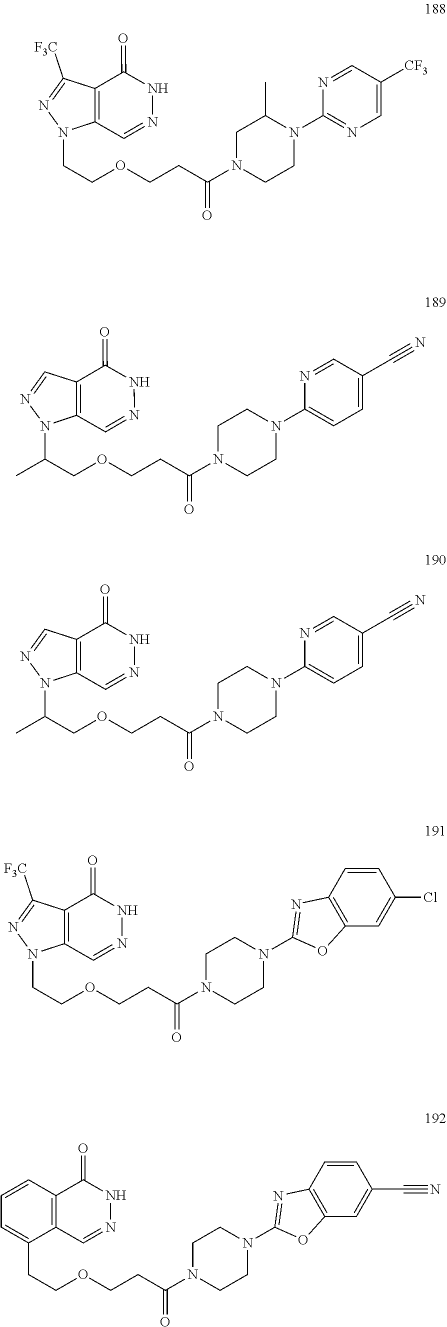

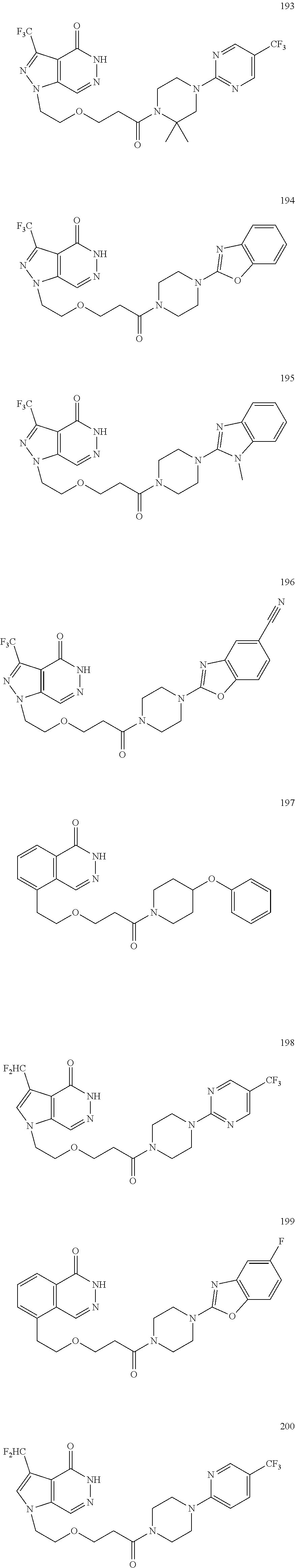

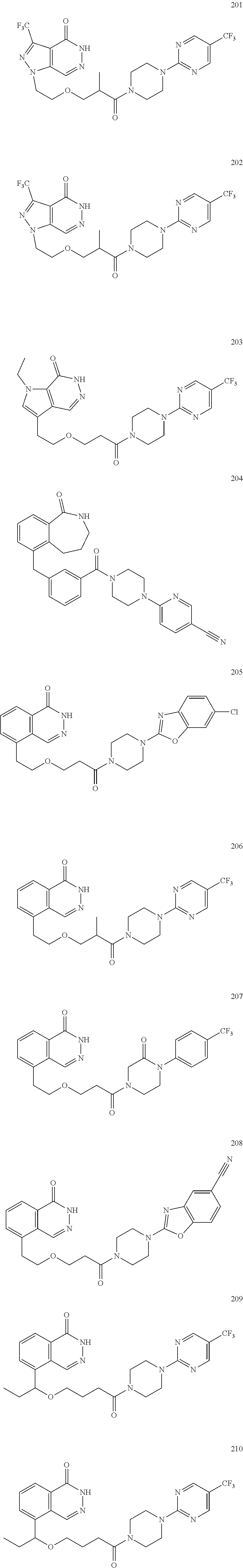

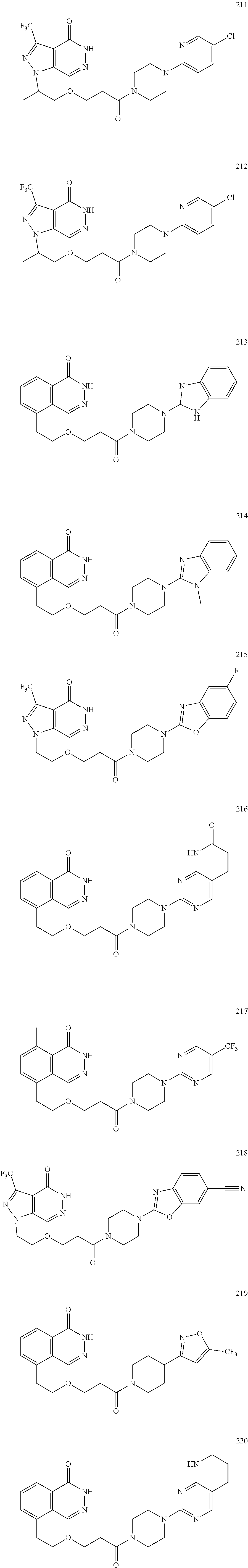

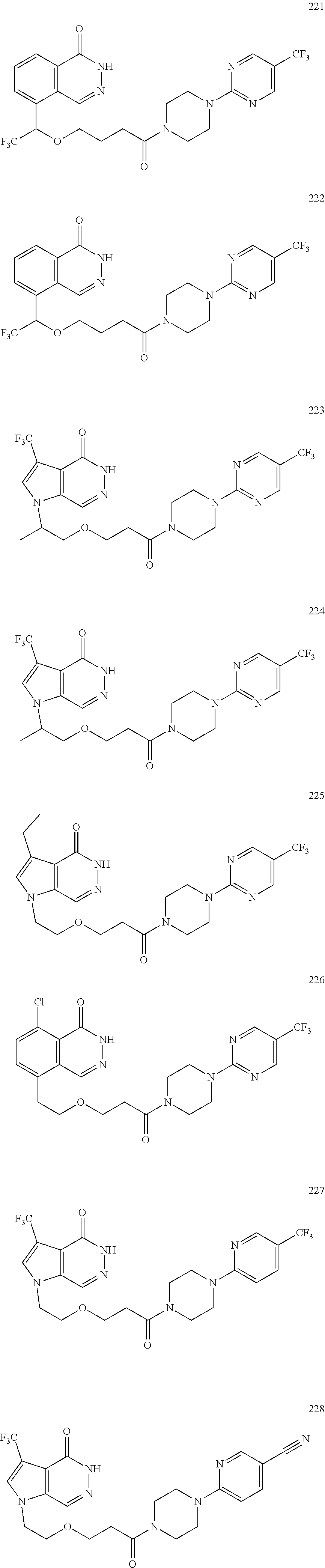

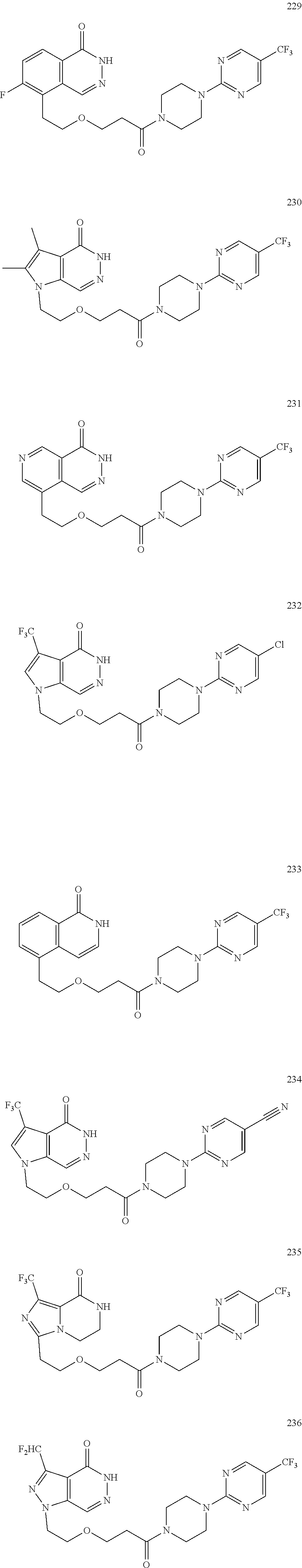

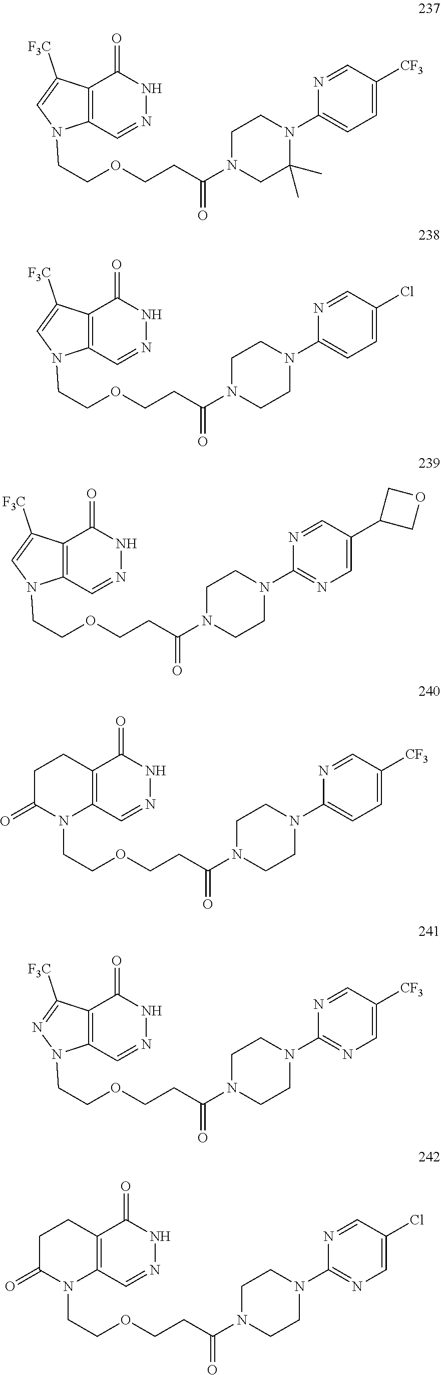 [SMILES: CC1(C)CN(C(=O)CCOCCn2cc(C(F)(F)F)c3c(=O)[nH]ncc32)CCN1c1ccc(C(F)(F)F)cn1 ; O=C(CCOCCn1cc(C(F)(F)F)c2c(=O)[nH]ncc21)N1CCN(c2ccc(Cl)cn2)CC1 ; O=C(CCOCCn1cc(C(F)(F)F)c2c(=O)[nH]ncc21)N1CCN(c2ncc(C3COC3)cn2)CC1 ; O=C(CCOCCN1C(=O)CCc2c1cn[nH]c2=O)N1CCN(c2ccc(C(F)(F)F)cn2)CC1 ; O=C(CCOCCn1nc(C(F)(F)F)c2c(=O)[nH]ncc21)N1CCN(c2ncc(C(F)(F)F)cn2)CC1 ; O=C(CCOCCN1C(=O)CCc2c1cn[nH]c2=O)N1CCN(c2ncc(Cl)cn2)CC1]

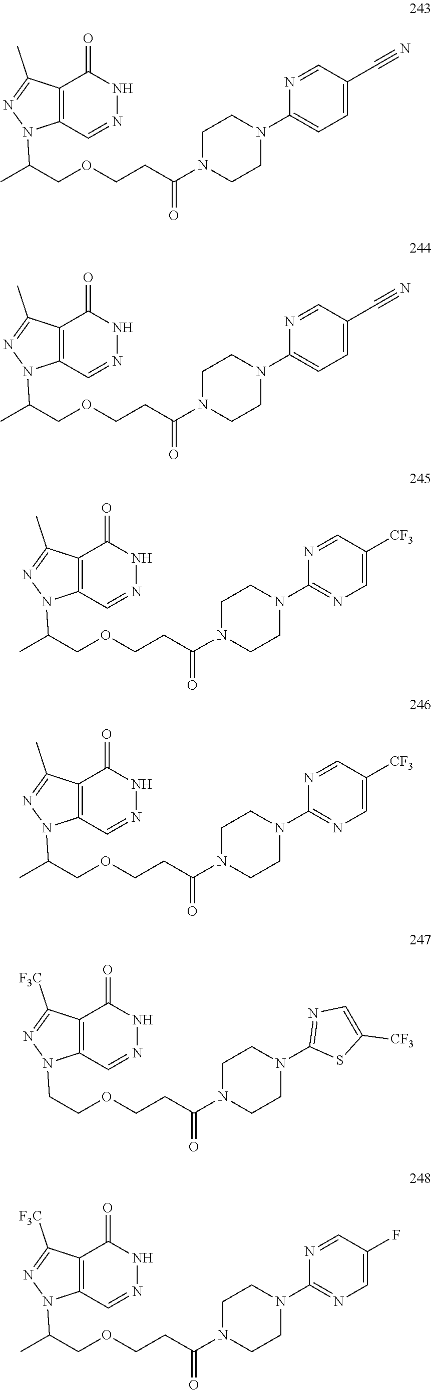 [SMILES: Cc1nn(C(C)COCCC(=O)N2CCN(c3ccc(C#N)cn3)CC2)c2cn[nH]c(=O)c12 ; Cc1nn(C(C)COCCC(=O)N2CCN(c3ccc(C#N)cn3)CC2)c2cn[nH]c(=O)c12 ; Cc1nn(C(C)COCCC(=O)N2CCN(c3ncc(C(F)(F)F)cn3)CC2)c2cn[nH]c(=O)c12 ; Cc1nn(C(C)COCCC(=O)N2CCN(c3ncc(C(F)(F)F)cn3)CC2)c2cn[nH]c(=O)c12 ; O=C(CCOCCn1nc(C(F)(F)F)c2c(=O)[nH]ncc21)N1CCN(c2ncc(C(F)(F)F)s2)CC1 ; CC(COCCC(=O)N1CCN(c2ncc(F)cn2)CC1)n1nc(C(F)(F)F)c2c(=O)[nH]ncc21]

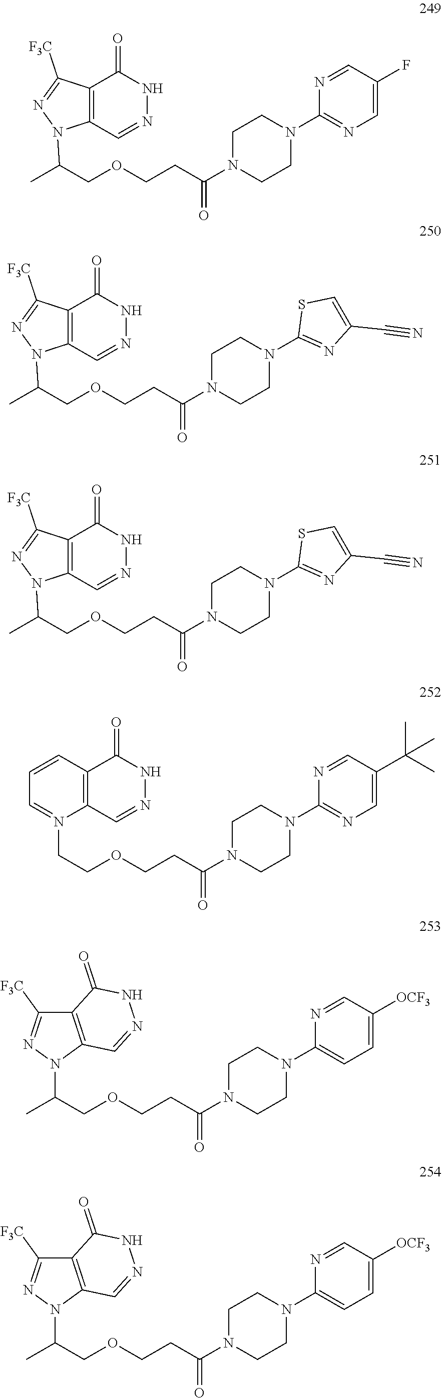

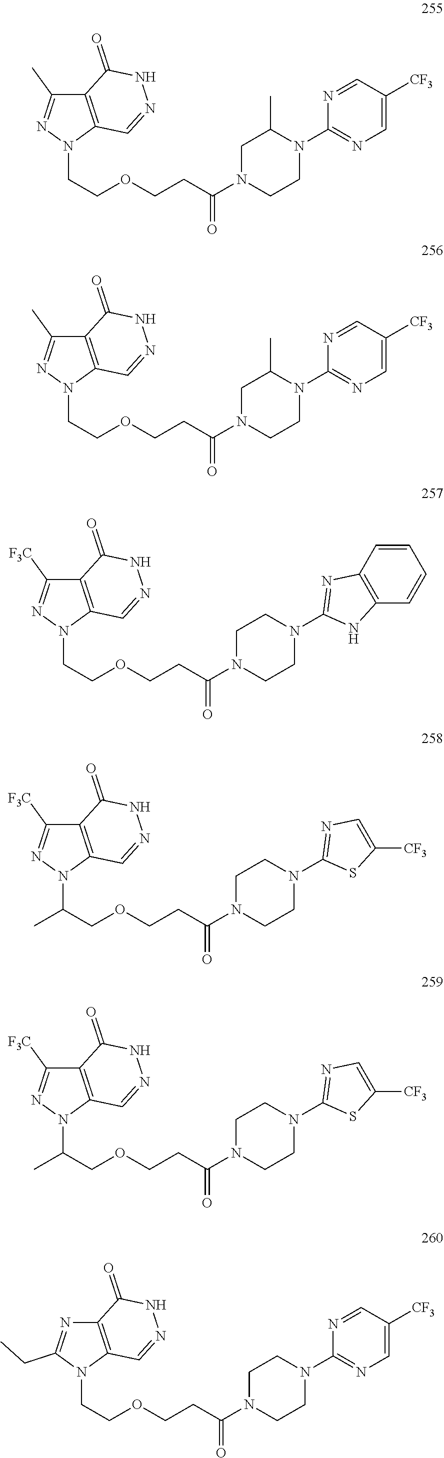 [SMILES: Cc1nn(CCOCCC(=O)N2CCN(c3ncc(C(F)(F)F)cn3)C(C)C2)c2cn[nH]c(=O)c12 ; Cc1nn(CCOCCC(=O)N2CCN(c3ncc(C(F)(F)F)cn3)C(C)C2)c2cn[nH]c(=O)c12 ; O=C(CCOCCn1nc(C(F)(F)F)c2c(=O)[nH]ncc21)N1CCN(c2nc3ccccc3[nH]2)CC1 ; CC(COCCC(=O)N1CCN(c2ncc(C(F)(F)F)s2)CC1)n1nc(C(F)(F)F)c2c(=O)[nH]ncc21 ; CC(COCCC(=O)N1CCN(c2ncc(C(F)(F)F)s2)CC1)n1nc(C(F)(F)F)c2c(=O)[nH]ncc21 ; CCc1nc2c(=O)[nH]ncc2n1CCOCCC(=O)N1CCN(c2ncc(C(F)(F)F)cn2)CC1]

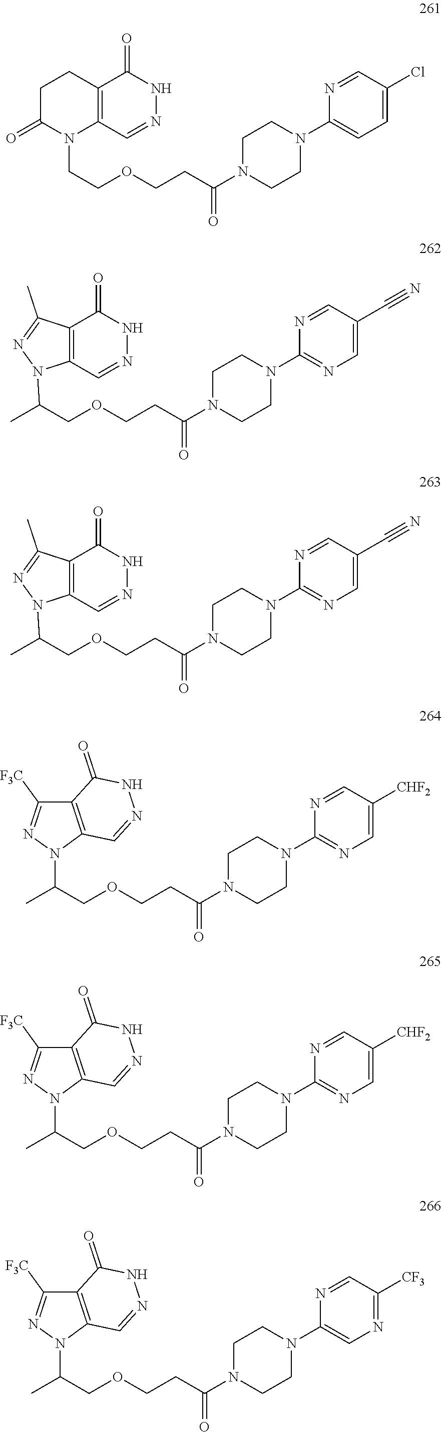 [SMILES: O=C(CCOCCN1C(=O)CCc2c1cn[nH]c2=O)N1CCN(c2ccc(Cl)cn2)CC1 ; Cc1nn(C(C)COCCC(=O)N2CCN(c3ncc(C#N)cn3)CC2)c2cn[nH]c(=O)c12 ; Cc1nn(C(C)COCCC(=O)N2CCN(c3ncc(C#N)cn3)CC2)c2cn[nH]c(=O)c12 ; CC(COCCC(=O)N1CCN(c2ncc(C(F)F)cn2)CC1)n1nc(C(F)(F)F)c2c(=O)[nH]ncc21 ; CC(COCCC(=O)N1CCN(c2ncc(C(F)F)cn2)CC1)n1nc(C(F)(F)F)c2c(=O)[nH]ncc21 ; CC(COCCC(=O)N1CCN(c2cnc(C(F)(F)F)cn2)CC1)n1nc(C(F)(F)F)c2c(=O)[nH]ncc21]

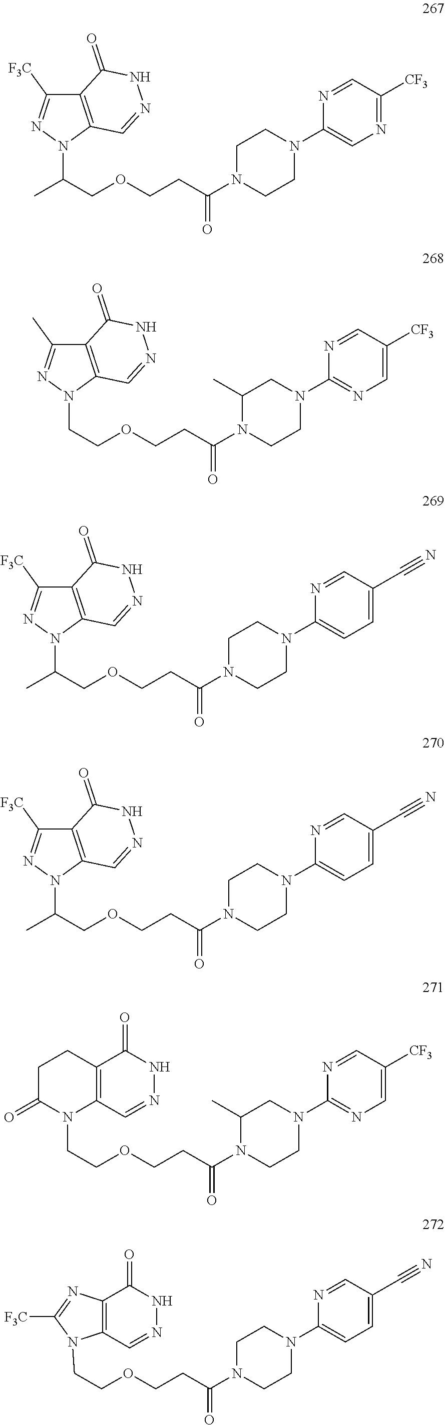 [SMILES: CC(COCCC(=O)N1CCN(c2cnc(C(F)(F)F)cn2)CC1)n1nc(C(F)(F)F)c2c(=O)[nH]ncc21 ; Cc1nn(CCOCCC(=O)N2CCN(c3ncc(C(F)(F)F)cn3)CC2C)c2cn[nH]c(=O)c12 ; CC(COCCC(=O)N1CCN(c2ccc(C#N)cn2)CC1)n1nc(C(F)(F)F)c2c(=O)[nH]ncc21 ; CC(COCCC(=O)N1CCN(c2ccc(C#N)cn2)CC1)n1nc(C(F)(F)F)c2c(=O)[nH]ncc21 ; CC1CN(c2ncc(C(F)(F)F)cn2)CCN1C(=O)CCOCCN1C(=O)CCc2c1cn[nH]c2=O ; N#Cc1ccc(N2CCN(C(=O)CCOCCn3c(C(F)(F)F)nc4c(=O)[nH]ncc43)CC2)nc1]

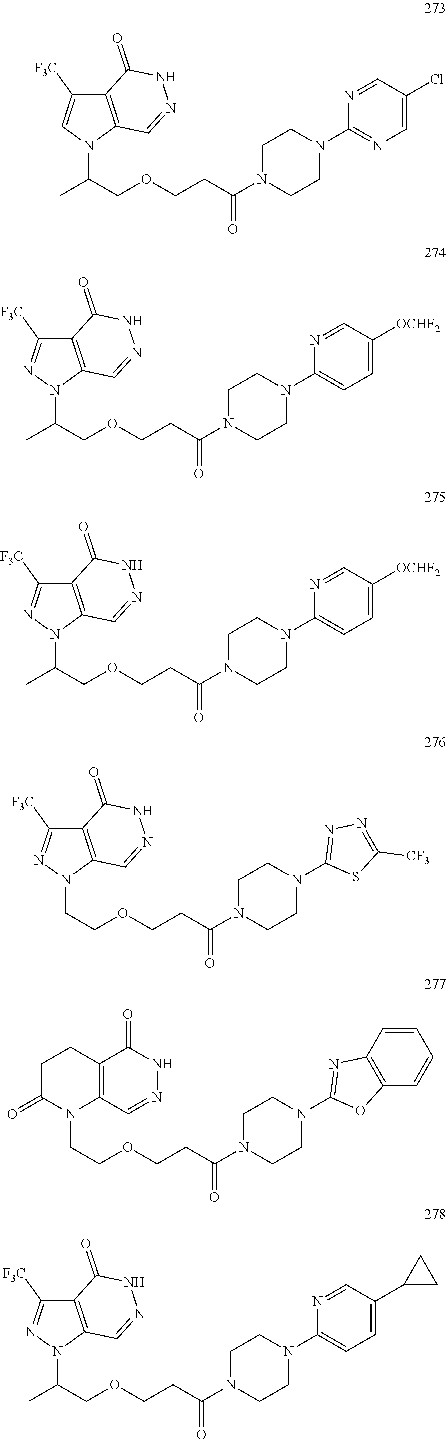 [SMILES: CC(COCCC(=O)N1CCN(c2ncc(Cl)cn2)CC1)n1cc(C(F)(F)F)c2c(=O)[nH]ncc21 ; CC(COCCC(=O)N1CCN(c2ccc(OC(F)F)cn2)CC1)n1nc(C(F)(F)F)c2c(=O)[nH]ncc21 ; CC(COCCC(=O)N1CCN(c2ccc(OC(F)F)cn2)CC1)n1nc(C(F)(F)F)c2c(=O)[nH]ncc21 ; O=C(CCOCCn1nc(C(F)(F)F)c2c(=O)[nH]ncc21)N1CCN(c2nnc(C(F)(F)F)s2)CC1 ; O=C(CCOCCN1C(=O)CCc2c1cn[nH]c2=O)N1CCN(c2nc3ccccc3o2)CC1 ; CC(COCCC(=O)N1CCN(c2ccc(C3CC3)cn2)CC1)n1nc(C(F)(F)F)c2c(=O)[nH]ncc21]

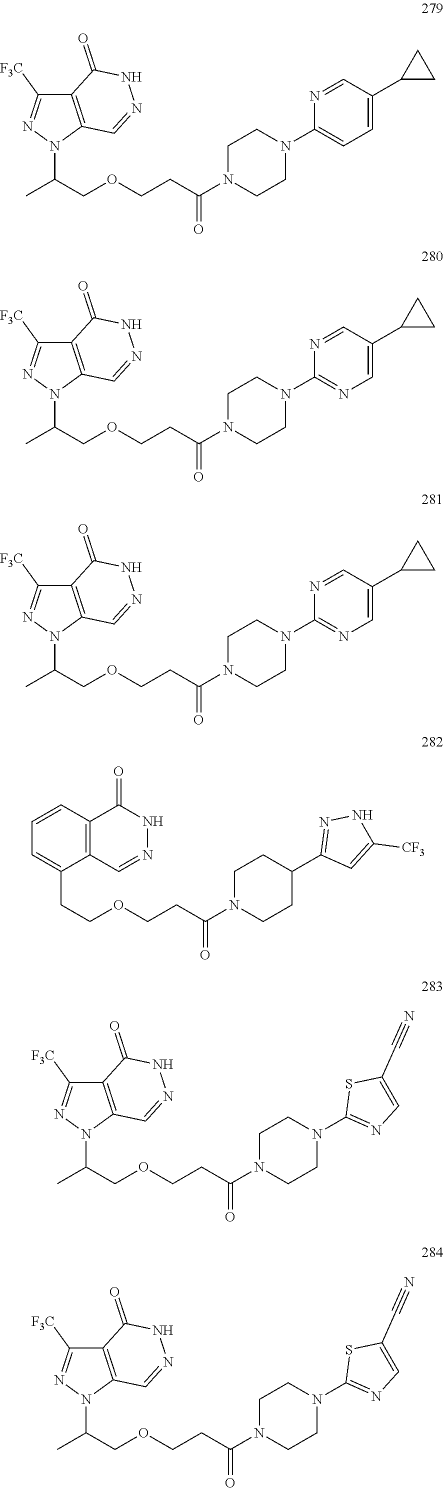 [SMILES: CC(COCCC(=O)N1CCN(c2ccc(C3CC3)cn2)CC1)n1nc(C(F)(F)F)c2c(=O)[nH]ncc21 ; CC(COCCC(=O)N1CCN(c2ncc(C3CC3)cn2)CC1)n1nc(C(F)(F)F)c2c(=O)[nH]ncc21 ; CC(COCCC(=O)N1CCN(c2ncc(C3CC3)cn2)CC1)n1nc(C(F)(F)F)c2c(=O)[nH]ncc21 ; O=C(CCOCCc1cccc2c(=O)[nH]ncc12)N1CCC(c2cc(C(F)(F)F)[nH]n2)CC1 ; CC(COCCC(=O)N1CCN(c2ncc(C#N)s2)CC1)n1nc(C(F)(F)F)c2c(=O)[nH]ncc21 ; CC(COCCC(=O)N1CCN(c2ncc(C#N)s2)CC1)n1nc(C(F)(F)F)c2c(=O)[nH]ncc21]

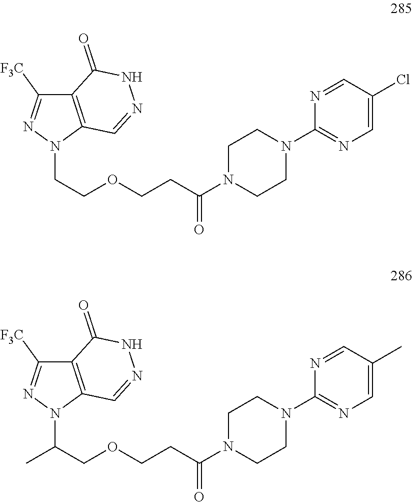 [SMILES: O=C(CCOCCn1nc(C(F)(F)F)c2c(=O)[nH]ncc21)N1CCN(c2ncc(Cl)cn2)CC1 ; Cc1cnc(N2CCN(C(=O)CCOCC(C)n3nc(C(F)(F)F)c4c(=O)[nH]ncc43)CC2)nc1]

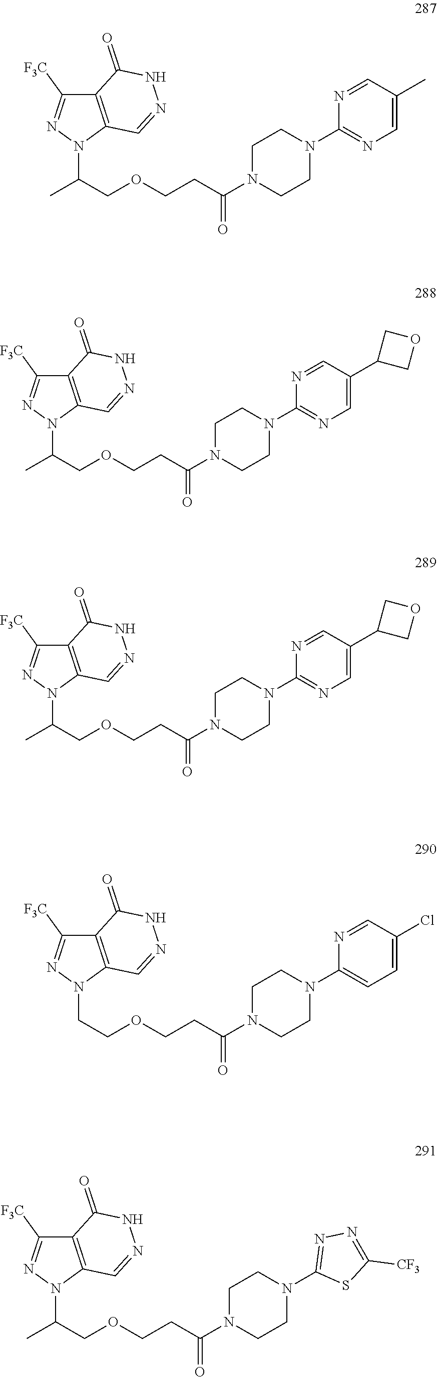 [SMILES: Cc1cnc(N2CCN(C(=O)CCOCC(C)n3nc(C(F)(F)F)c4c(=O)[nH]ncc43)CC2)nc1 ; CC(COCCC(=O)N1CCN(c2ncc(C3COC3)cn2)CC1)n1nc(C(F)(F)F)c2c(=O)[nH]ncc21 ; CC(COCCC(=O)N1CCN(c2ncc(C3COC3)cn2)CC1)n1nc(C(F)(F)F)c2c(=O)[nH]ncc21 ; O=C(CCOCCn1nc(C(F)(F)F)c2c(=O)[nH]ncc21)N1CCN(c2ccc(Cl)cn2)CC1 ; CC(COCCC(=O)N1CCN(c2nnc(C(F)(F)F)s2)CC1)n1nc(C(F)(F)F)c2c(=O)[nH]ncc21]

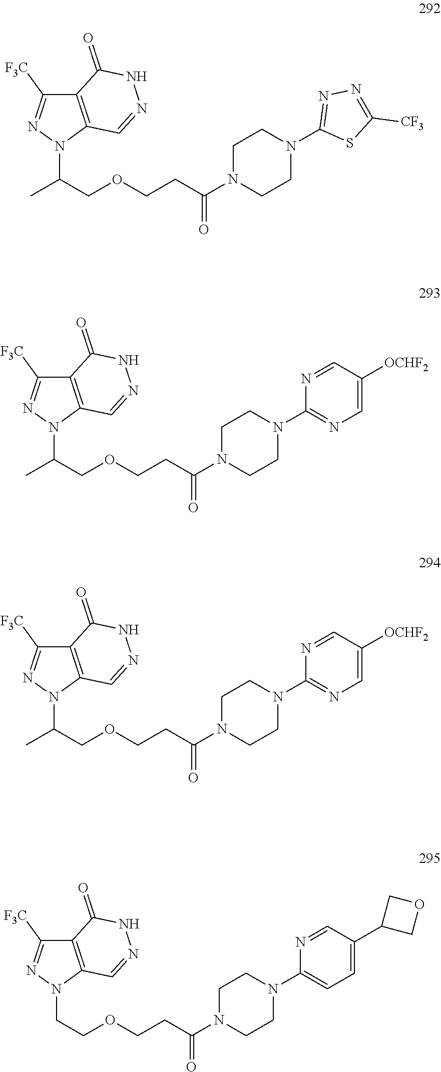 [SMILES: CC(COCCC(=O)N1CCN(c2nnc(C(F)(F)F)s2)CC1)n1nc(C(F)(F)F)c2c(=O)[nH]ncc21 ; CC(COCCC(=O)N1CCN(c2ncc(OC(F)F)cn2)CC1)n1nc(C(F)(F)F)c2c(=O)[nH]ncc21 ; CC(COCCC(=O)N1CCN(c2ncc(OC(F)F)cn2)CC1)n1nc(C(F)(F)F)c2c(=O)[nH]ncc21 ; O=C(CCOCCn1nc(C(F)(F)F)c2c(=O)[nH]ncc21)N1CCN(c2ccc(C3COC3)cn2)CC1]

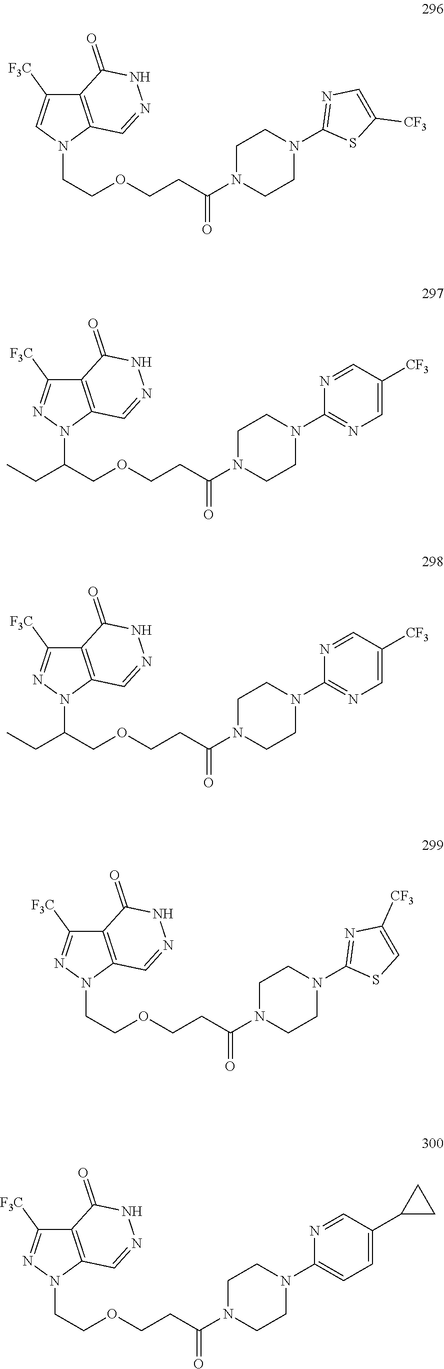 [SMILES: O=C(CCOCCn1cc(C(F)(F)F)c2c(=O)[nH]ncc21)N1CCN(c2ncc(C(F)(F)F)s2)CC1 ; CCC(COCCC(=O)N1CCN(c2ncc(C(F)(F)F)cn2)CC1)n1nc(C(F)(F)F)c2c(=O)[nH]ncc21 ; CCC(COCCC(=O)N1CCN(c2ncc(C(F)(F)F)cn2)CC1)n1nc(C(F)(F)F)c2c(=O)[nH]ncc21 ; O=C(CCOCCn1nc(C(F)(F)F)c2c(=O)[nH]ncc21)N1CCN(c2nc(C(F)(F)F)cs2)CC1 ; O=C(CCOCCn1nc(C(F)(F)F)c2c(=O)[nH]ncc21)N1CCN(c2ccc(C3CC3)cn2)CC1]

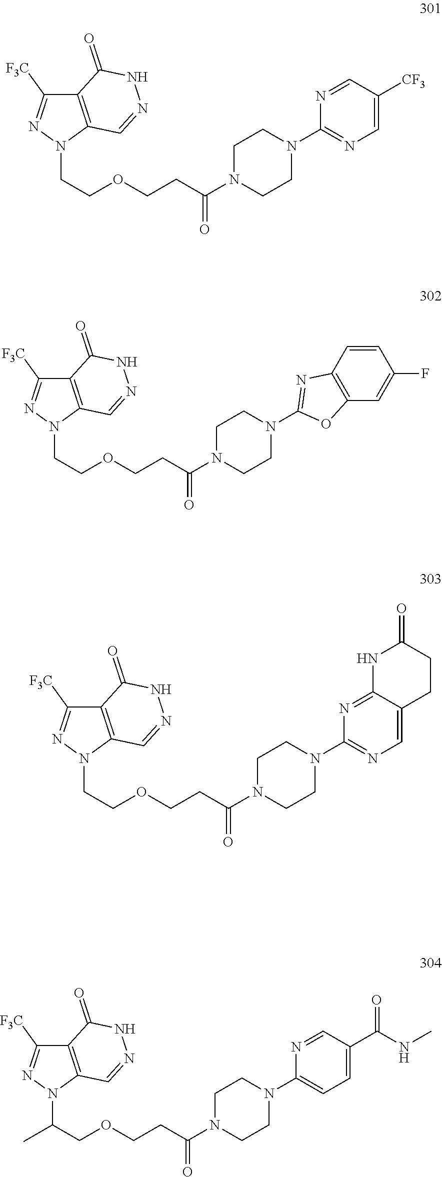 [SMILES: O=C(CCOCCn1nc(C(F)(F)F)c2c(=O)[nH]ncc21)N1CCN(c2ncc(C(F)(F)F)cn2)CC1 ; O=C(CCOCCn1nc(C(F)(F)F)c2c(=O)[nH]ncc21)N1CCN(c2nc3ccc(F)cc3o2)CC1 ; O=C1CCc2cnc(N3CCN(C(=O)CCOCCn4nc(C(F)(F)F)c5c(=O)[nH]ncc54)CC3)nc2N1 ; CNC(=O)c1ccc(N2CCN(C(=O)CCOCC(C)n3nc(C(F)(F)F)c4c(=O)[nH]ncc43)CC2)nc1]

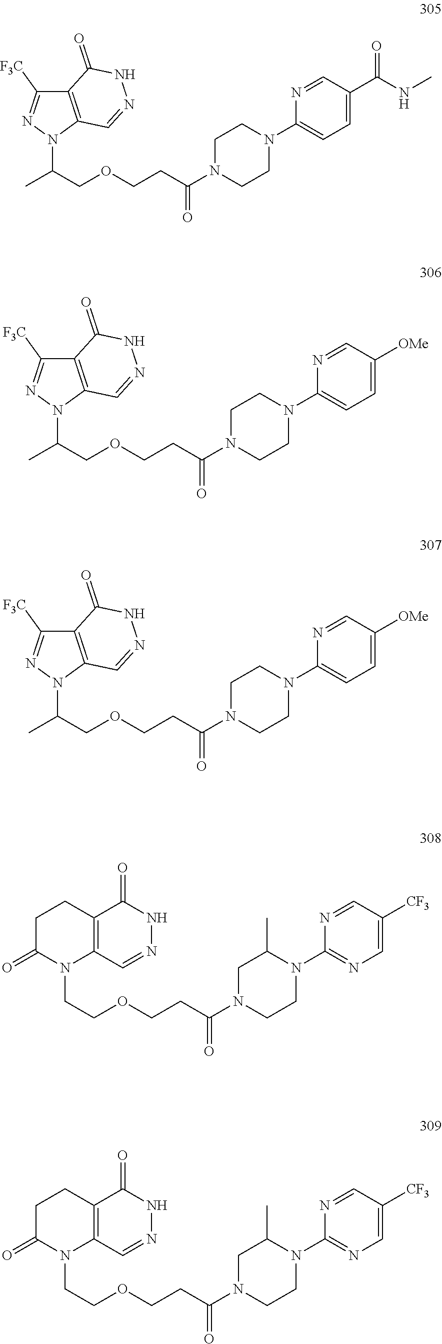 [SMILES: CNC(=O)c1ccc(N2CCN(C(=O)CCOCC(C)n3nc(C(F)(F)F)c4c(=O)[nH]ncc43)CC2)nc1 ; COc1ccc(N2CCN(C(=O)CCOCC(C)n3nc(C(F)(F)F)c4c(=O)[nH]ncc43)CC2)nc1 ; COc1ccc(N2CCN(C(=O)CCOCC(C)n3nc(C(F)(F)F)c4c(=O)[nH]ncc43)CC2)nc1 ; CC1CN(C(=O)CCOCCN2C(=O)CCc3c2cn[nH]c3=O)CCN1c1ncc(C(F)(F)F)cn1 ; CC1CN(C(=O)CCOCCN2C(=O)CCc3c2cn[nH]c3=O)CCN1c1ncc(C(F)(F)F)cn1]

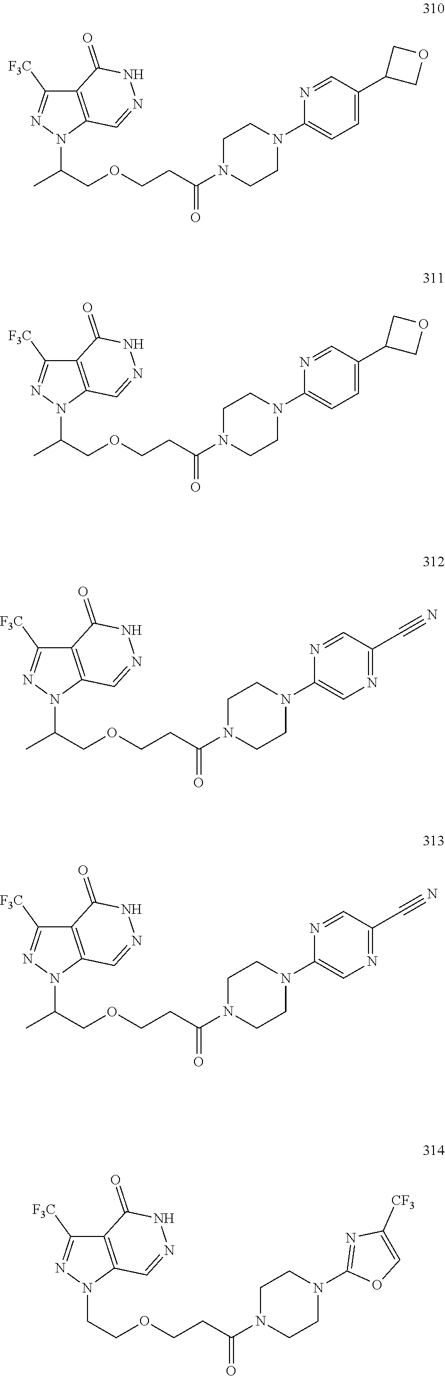 [SMILES: CC(COCCC(=O)N1CCN(c2ccc(C3COC3)cn2)CC1)n1nc(C(F)(F)F)c2c(=O)[nH]ncc21 ; CC(COCCC(=O)N1CCN(c2ccc(C3COC3)cn2)CC1)n1nc(C(F)(F)F)c2c(=O)[nH]ncc21 ; CC(COCCC(=O)N1CCN(c2cnc(C#N)cn2)CC1)n1nc(C(F)(F)F)c2c(=O)[nH]ncc21 ; CC(COCCC(=O)N1CCN(c2cnc(C#N)cn2)CC1)n1nc(C(F)(F)F)c2c(=O)[nH]ncc21 ; O=C(CCOCCn1nc(C(F)(F)F)c2c(=O)[nH]ncc21)N1CCN(c2nc(C(F)(F)F)co2)CC1]

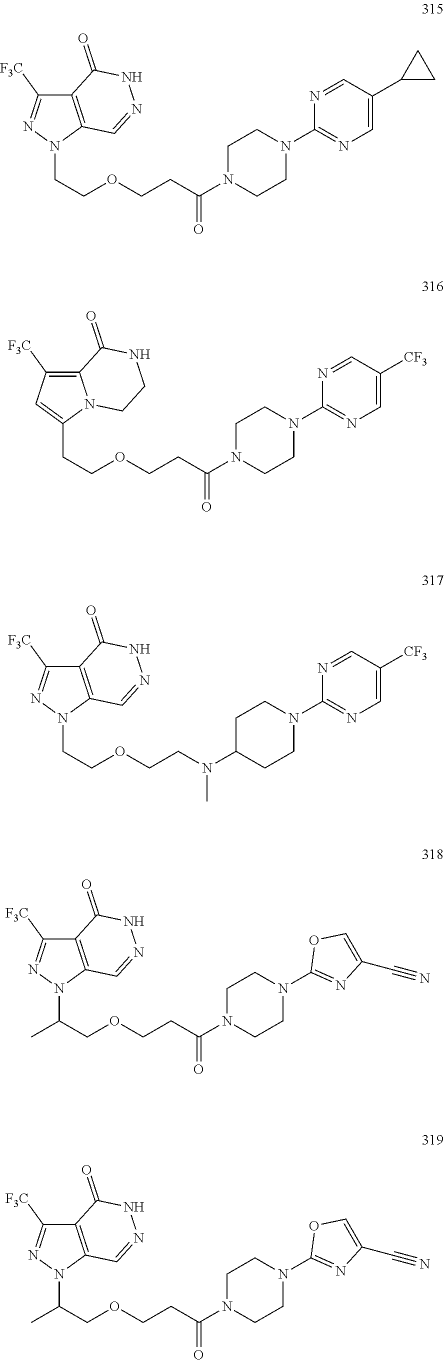 [SMILES: O=C(CCOCCn1nc(C(F)(F)F)c2c(=O)[nH]ncc21)N1CCN(c2ncc(C3CC3)cn2)CC1 ; O=C1NCCn2c(CCOCCC(=O)N3CCN(c4ncc(C(F)(F)F)cn4)CC3)cc(C(F)(F)F)c21 ; CN(CCOCCn1nc(C(F)(F)F)c2c(=O)[nH]ncc21)C1CCN(c2ncc(C(F)(F)F)cn2)CC1 ; CC(COCCC(=O)N1CCN(c2nc(C#N)co2)CC1)n1nc(C(F)(F)F)c2c(=O)[nH]ncc21 ; CC(COCCC(=O)N1CCN(c2nc(C#N)co2)CC1)n1nc(C(F)(F)F)c2c(=O)[nH]ncc21]

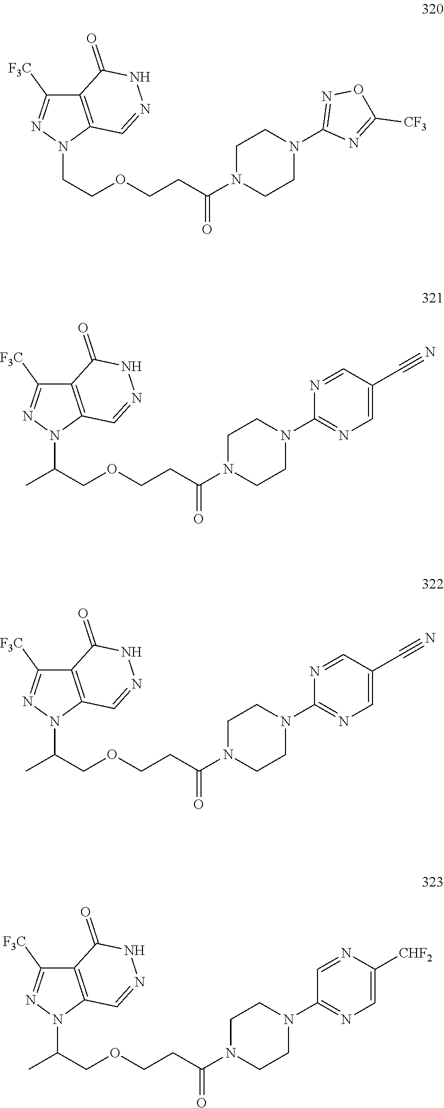 [SMILES: O=C(CCOCCn1nc(C(F)(F)F)c2c(=O)[nH]ncc21)N1CCN(c2noc(C(F)(F)F)n2)CC1 ; CC(COCCC(=O)N1CCN(c2ncc(C#N)cn2)CC1)n1nc(C(F)(F)F)c2c(=O)[nH]ncc21 ; CC(COCCC(=O)N1CCN(c2ncc(C#N)cn2)CC1)n1nc(C(F)(F)F)c2c(=O)[nH]ncc21 ; CC(COCCC(=O)N1CCN(c2cnc(C(F)F)cn2)CC1)n1nc(C(F)(F)F)c2c(=O)[nH]ncc21]

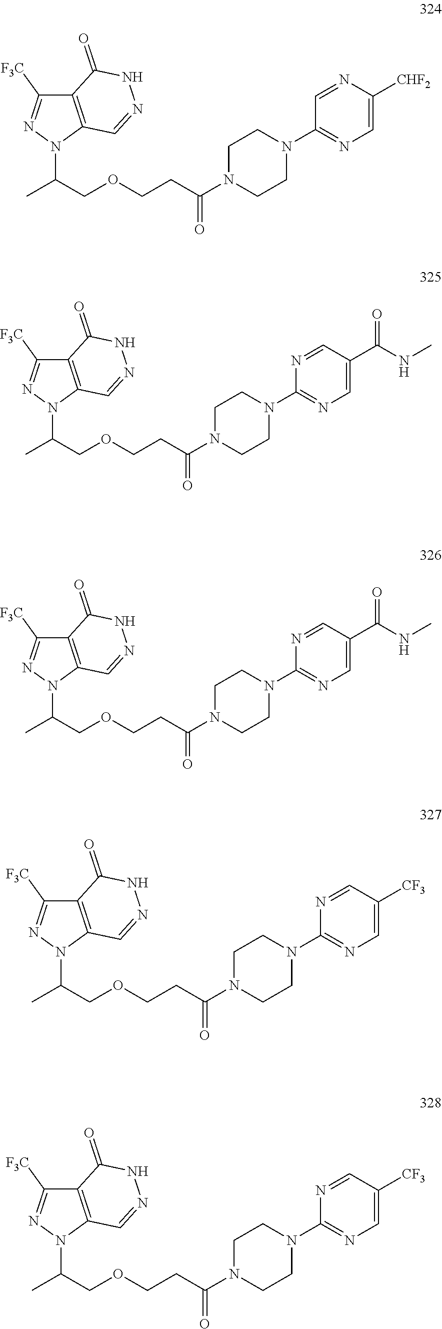 [SMILES: CC(COCCC(=O)N1CCN(c2cnc(C(F)F)cn2)CC1)n1nc(C(F)(F)F)c2c(=O)[nH]ncc21 ; CNC(=O)c1cnc(N2CCN(C(=O)CCOCC(C)n3nc(C(F)(F)F)c4c(=O)[nH]ncc43)CC2)nc1 ; CNC(=O)c1cnc(N2CCN(C(=O)CCOCC(C)n3nc(C(F)(F)F)c4c(=O)[nH]ncc43)CC2)nc1 ; CC(COCCC(=O)N1CCN(c2ncc(C(F)(F)F)cn2)CC1)n1nc(C(F)(F)F)c2c(=O)[nH]ncc21 ; CC(COCCC(=O)N1CCN(c2ncc(C(F)(F)F)cn2)CC1)n1nc(C(F)(F)F)c2c(=O)[nH]ncc21]

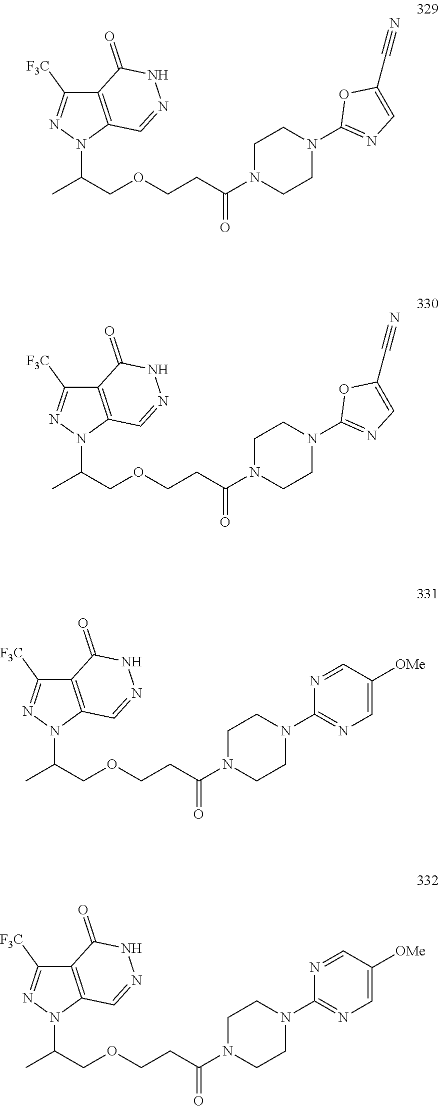 [SMILES: CC(COCCC(=O)N1CCN(c2ncc(C#N)o2)CC1)n1nc(C(F)(F)F)c2c(=O)[nH]ncc21 ; CC(COCCC(=O)N1CCN(c2ncc(C#N)o2)CC1)n1nc(C(F)(F)F)c2c(=O)[nH]ncc21 ; COc1cnc(N2CCN(C(=O)CCOCC(C)n3nc(C(F)(F)F)c4c(=O)[nH]ncc43)CC2)nc1 ; COc1cnc(N2CCN(C(=O)CCOCC(C)n3nc(C(F)(F)F)c4c(=O)[nH]ncc43)CC2)nc1]

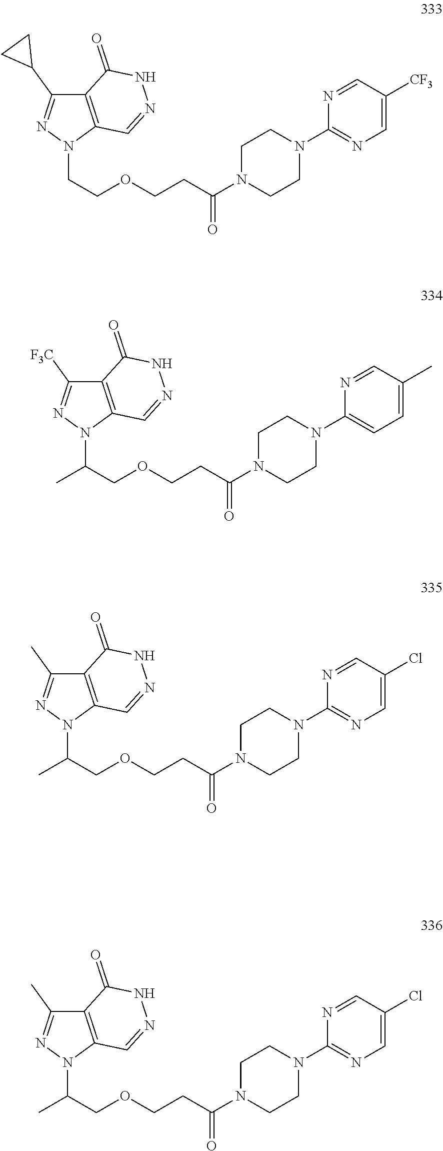

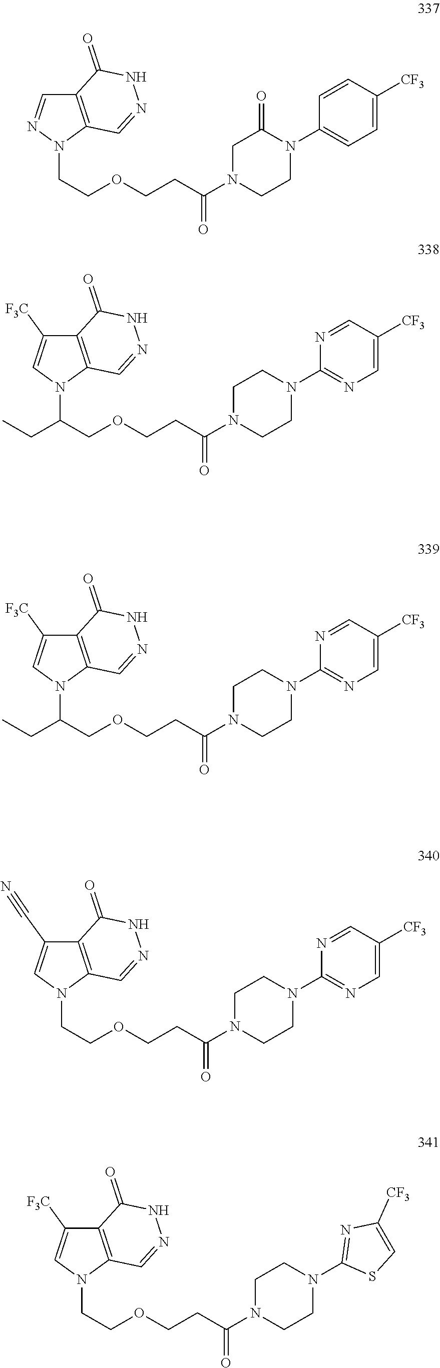 [SMILES: O=C(CCOCCn1ncc2c(=O)[nH]ncc21)N1CCN(c2ccc(C(F)(F)F)cc2)C(=O)C1 ; CCC(COCCC(=O)N1CCN(c2ncc(C(F)(F)F)cn2)CC1)n1cc(C(F)(F)F)c2c(=O)[nH]ncc21 ; CCC(COCCC(=O)N1CCN(c2ncc(C(F)(F)F)cn2)CC1)n1cc(C(F)(F)F)c2c(=O)[nH]ncc21 ; N#Cc1cn(CCOCCC(=O)N2CCN(c3ncc(C(F)(F)F)cn3)CC2)c2cn[nH]c(=O)c12 ; O=C(CCOCCn1cc(C(F)(F)F)c2c(=O)[nH]ncc21)N1CCN(c2nc(C(F)(F)F)cs2)CC1]

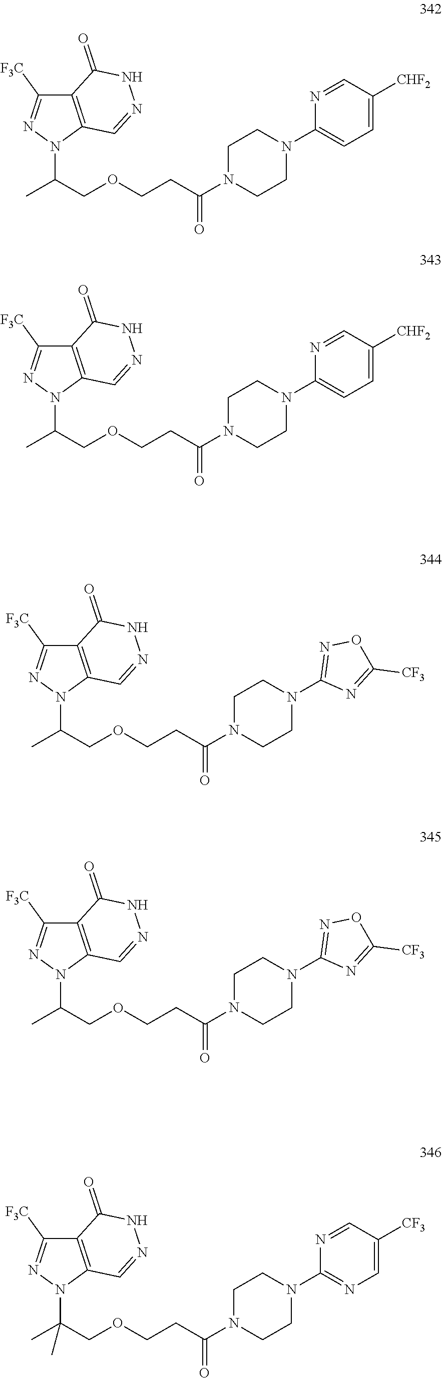 [SMILES: CC(COCCC(=O)N1CCN(c2ccc(C(F)F)cn2)CC1)n1nc(C(F)(F)F)c2c(=O)[nH]ncc21 ; CC(COCCC(=O)N1CCN(c2ccc(C(F)F)cn2)CC1)n1nc(C(F)(F)F)c2c(=O)[nH]ncc21 ; CC(COCCC(=O)N1CCN(c2noc(C(F)(F)F)n2)CC1)n1nc(C(F)(F)F)c2c(=O)[nH]ncc21 ; CC(COCCC(=O)N1CCN(c2noc(C(F)(F)F)n2)CC1)n1nc(C(F)(F)F)c2c(=O)[nH]ncc21 ; CC(C)(COCCC(=O)N1CCN(c2ncc(C(F)(F)F)cn2)CC1)n1nc(C(F)(F)F)c2c(=O)[nH]ncc21]

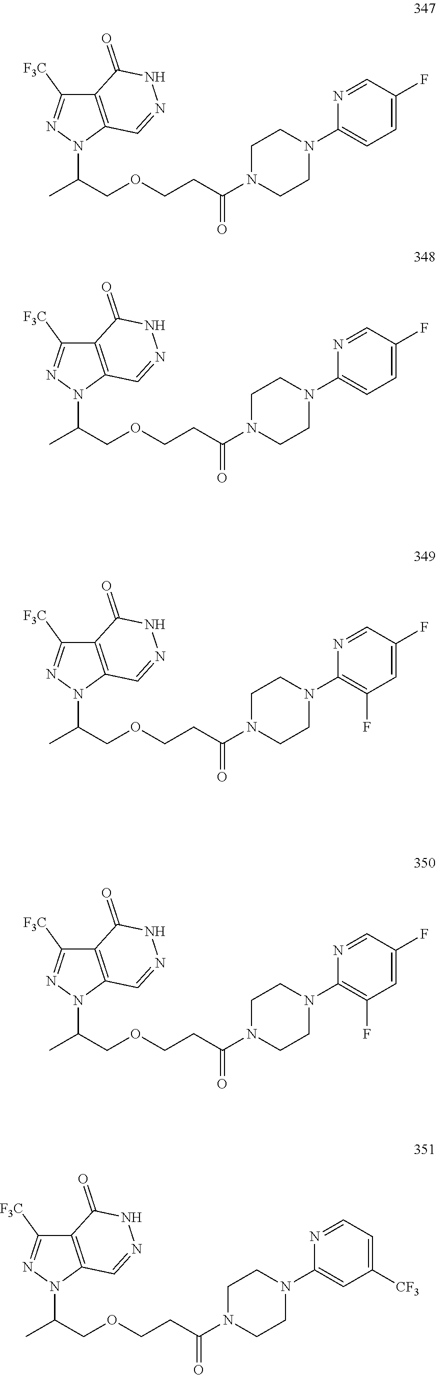 [SMILES: CC(COCCC(=O)N1CCN(c2ccc(F)cn2)CC1)n1nc(C(F)(F)F)c2c(=O)[nH]ncc21 ; CC(COCCC(=O)N1CCN(c2ccc(F)cn2)CC1)n1nc(C(F)(F)F)c2c(=O)[nH]ncc21 ; CC(COCCC(=O)N1CCN(c2ncc(F)cc2F)CC1)n1nc(C(F)(F)F)c2c(=O)[nH]ncc21 ; CC(COCCC(=O)N1CCN(c2ncc(F)cc2F)CC1)n1nc(C(F)(F)F)c2c(=O)[nH]ncc21 ; CC(COCCC(=O)N1CCN(c2cc(C(F)(F)F)ccn2)CC1)n1nc(C(F)(F)F)c2c(=O)[nH]ncc21]

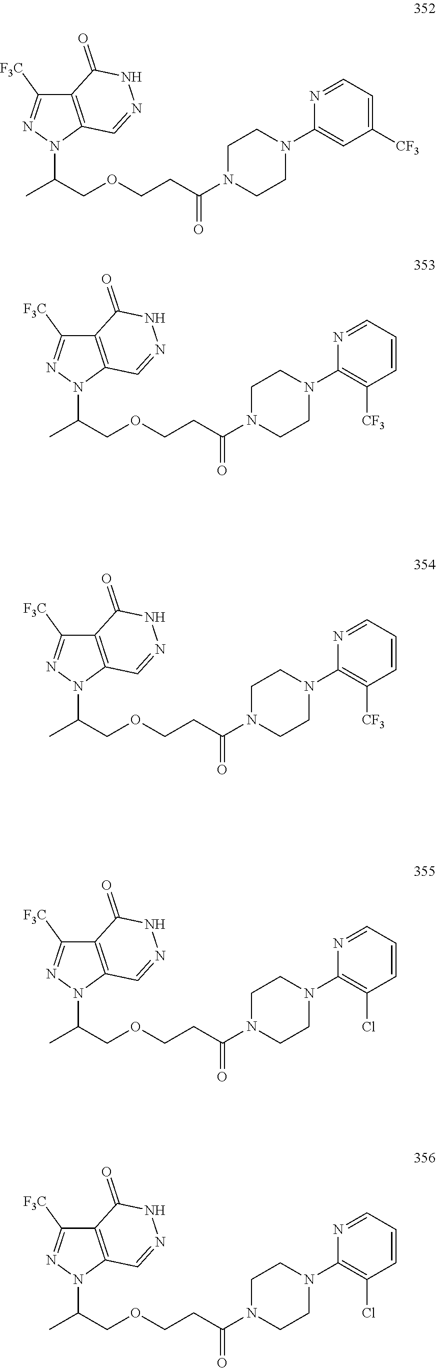 [SMILES: CC(COCCC(=O)N1CCN(c2cc(C(F)(F)F)ccn2)CC1)n1nc(C(F)(F)F)c2c(=O)[nH]ncc21 ; CC(COCCC(=O)N1CCN(c2ncccc2C(F)(F)F)CC1)n1nc(C(F)(F)F)c2c(=O)[nH]ncc21 ; CC(COCCC(=O)N1CCN(c2ncccc2C(F)(F)F)CC1)n1nc(C(F)(F)F)c2c(=O)[nH]ncc21 ; CC(COCCC(=O)N1CCN(c2ncccc2Cl)CC1)n1nc(C(F)(F)F)c2c(=O)[nH]ncc21 ; CC(COCCC(=O)N1CCN(c2ncccc2Cl)CC1)n1nc(C(F)(F)F)c2c(=O)[nH]ncc21]

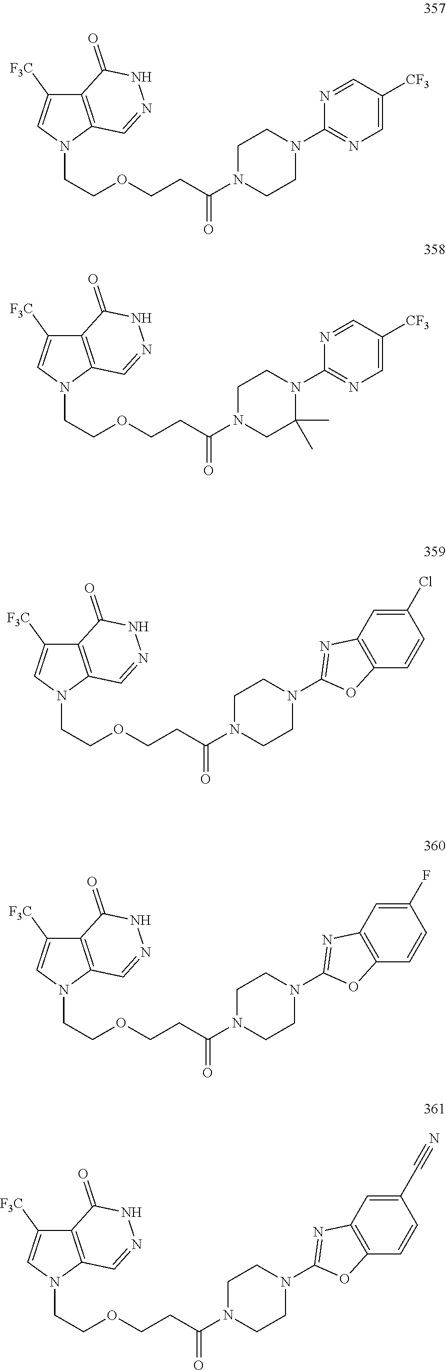 [SMILES: O=C(CCOCCn1cc(C(F)(F)F)c2c(=O)[nH]ncc21)N1CCN(c2ncc(C(F)(F)F)cn2)CC1 ; CC1(C)CN(C(=O)CCOCCn2cc(C(F)(F)F)c3c(=O)[nH]ncc32)CCN1c1ncc(C(F)(F)F)cn1 ; O=C(CCOCCn1cc(C(F)(F)F)c2c(=O)[nH]ncc21)N1CCN(c2nc3cc(Cl)ccc3o2)CC1 ; O=C(CCOCCn1cc(C(F)(F)F)c2c(=O)[nH]ncc21)N1CCN(c2nc3cc(F)ccc3o2)CC1 ; N#Cc1ccc2oc(N3CCN(C(=O)CCOCCn4cc(C(F)(F)F)c5c(=O)[nH]ncc54)CC3)nc2c1]

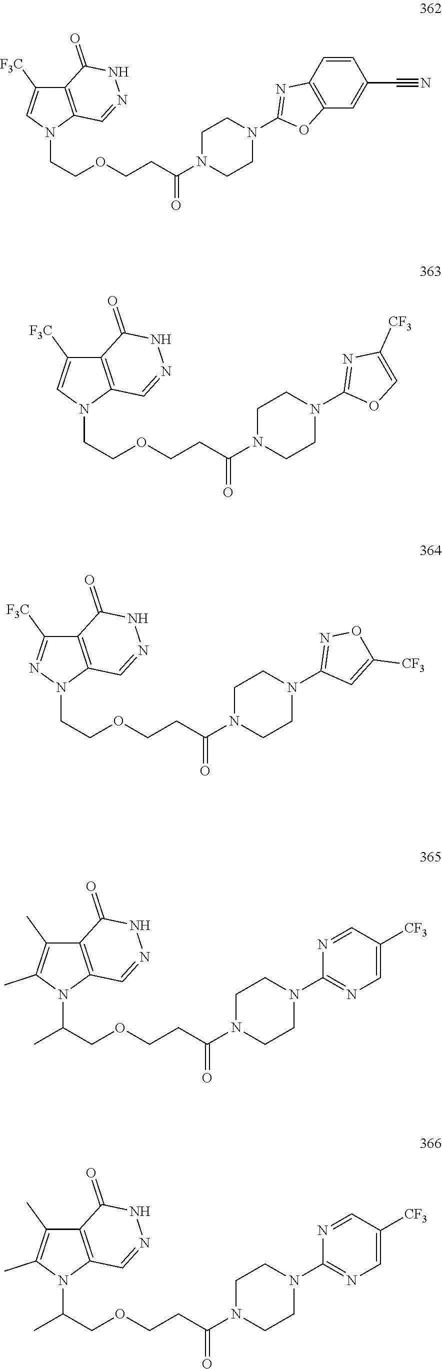 [SMILES: N#Cc1ccc2nc(N3CCN(C(=O)CCOCCn4cc(C(F)(F)F)c5c(=O)[nH]ncc54)CC3)oc2c1 ; O=C(CCOCCn1cc(C(F)(F)F)c2c(=O)[nH]ncc21)N1CCN(c2nc(C(F)(F)F)co2)CC1 ; O=C(CCOCCn1nc(C(F)(F)F)c2c(=O)[nH]ncc21)N1CCN(c2cc(C(F)(F)F)on2)CC1 ; Cc1c(C)n(C(C)COCCC(=O)N2CCN(c3ncc(C(F)(F)F)cn3)CC2)c2cn[nH]c(=O)c12 ; Cc1c(C)n(C(C)COCCC(=O)N2CCN(c3ncc(C(F)(F)F)cn3)CC2)c2cn[nH]c(=O)c12]

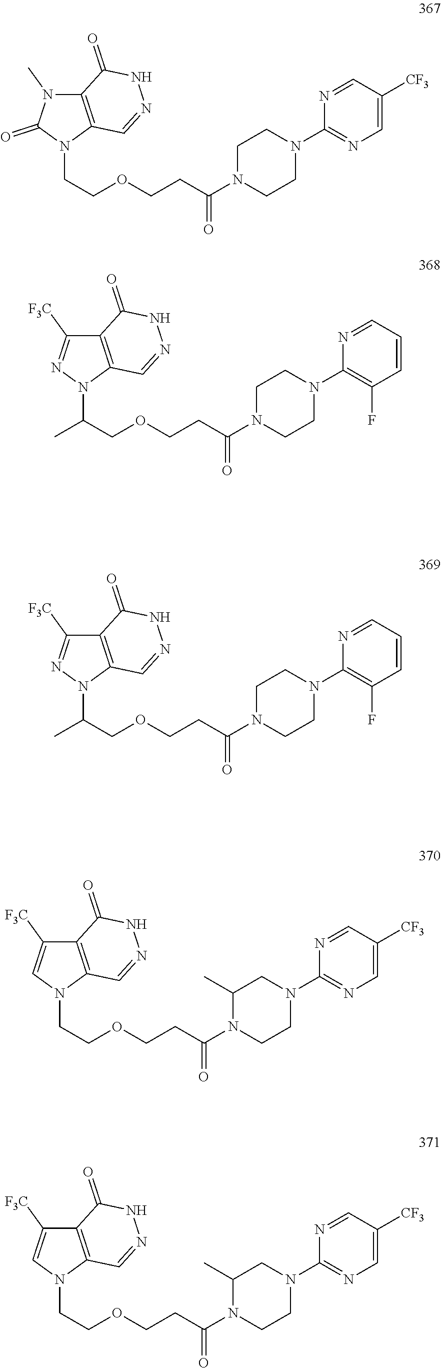 [SMILES: Cn1c(=O)n(CCOCCC(=O)N2CCN(c3ncc(C(F)(F)F)cn3)CC2)c2cn[nH]c(=O)c21 ; CC(COCCC(=O)N1CCN(c2ncccc2F)CC1)n1nc(C(F)(F)F)c2c(=O)[nH]ncc21 ; CC(COCCC(=O)N1CCN(c2ncccc2F)CC1)n1nc(C(F)(F)F)c2c(=O)[nH]ncc21 ; CC1CN(c2ncc(C(F)(F)F)cn2)CCN1C(=O)CCOCCn1cc(C(F)(F)F)c2c(=O)[nH]ncc21 ; CC1CN(c2ncc(C(F)(F)F)cn2)CCN1C(=O)CCOCCn1cc(C(F)(F)F)c2c(=O)[nH]ncc21]

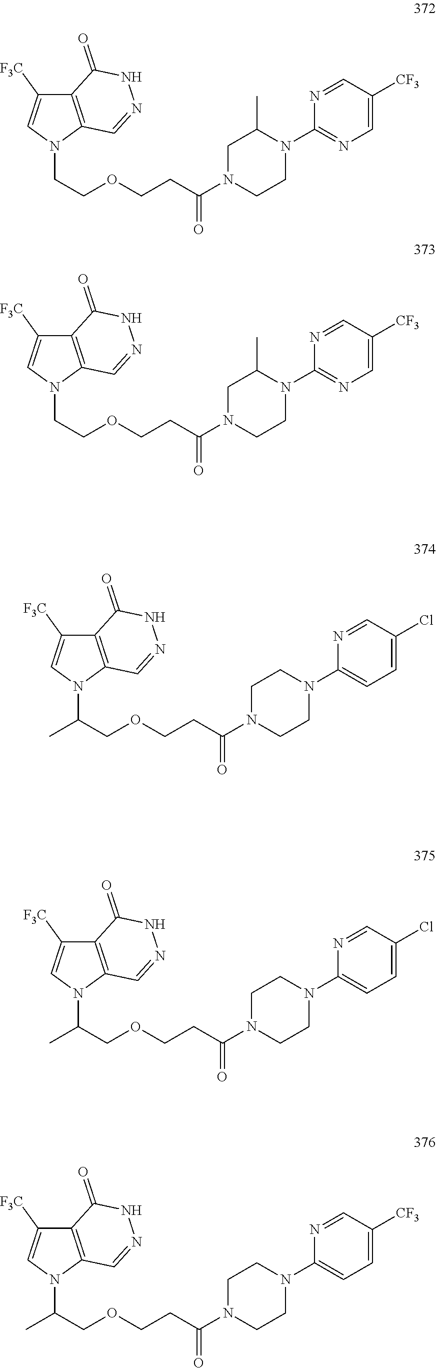 [SMILES: CC1CN(C(=O)CCOCCn2cc(C(F)(F)F)c3c(=O)[nH]ncc32)CCN1c1ncc(C(F)(F)F)cn1 ; CC1CN(C(=O)CCOCCn2cc(C(F)(F)F)c3c(=O)[nH]ncc32)CCN1c1ncc(C(F)(F)F)cn1 ; CC(COCCC(=O)N1CCN(c2ccc(Cl)cn2)CC1)n1cc(C(F)(F)F)c2c(=O)[nH]ncc21 ; CC(COCCC(=O)N1CCN(c2ccc(Cl)cn2)CC1)n1cc(C(F)(F)F)c2c(=O)[nH]ncc21 ; CC(COCCC(=O)N1CCN(c2ccc(C(F)(F)F)cn2)CC1)n1cc(C(F)(F)F)c2c(=O)[nH]ncc21]

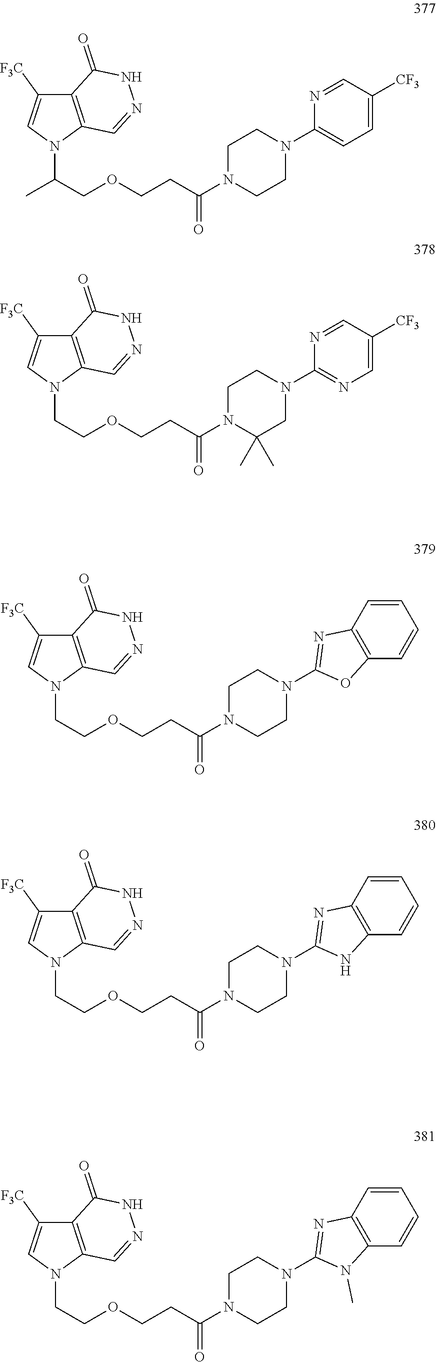 [SMILES: CC(COCCC(=O)N1CCN(c2ccc(C(F)(F)F)cn2)CC1)n1cc(C(F)(F)F)c2c(=O)[nH]ncc21 ; CC1(C)CN(c2ncc(C(F)(F)F)cn2)CCN1C(=O)CCOCCn1cc(C(F)(F)F)c2c(=O)[nH]ncc21 ; O=C(CCOCCn1cc(C(F)(F)F)c2c(=O)[nH]ncc21)N1CCN(c2nc3ccccc3o2)CC1 ; O=C(CCOCCn1cc(C(F)(F)F)c2c(=O)[nH]ncc21)N1CCN(c2nc3ccccc3[nH]2)CC1 ; Cn1c(N2CCN(C(=O)CCOCCn3cc(C(F)(F)F)c4c(=O)[nH]ncc43)CC2)nc2ccccc21]

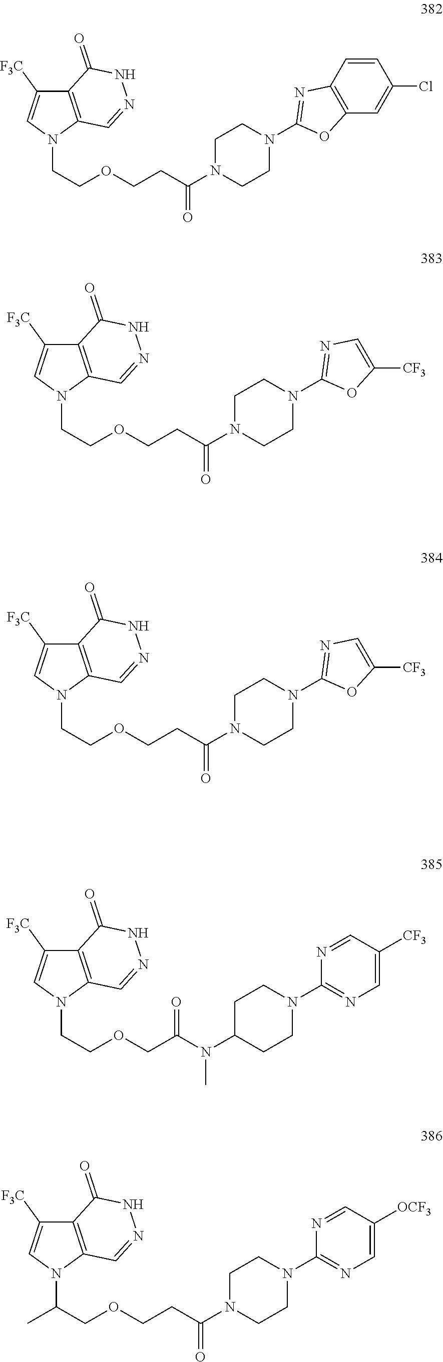 [SMILES: O=C(CCOCCn1cc(C(F)(F)F)c2c(=O)[nH]ncc21)N1CCN(c2nc3ccc(Cl)cc3o2)CC1 ; O=C(CCOCCn1cc(C(F)(F)F)c2c(=O)[nH]ncc21)N1CCN(c2ncc(C(F)(F)F)o2)CC1 ; O=C(CCOCCn1cc(C(F)(F)F)c2c(=O)[nH]ncc21)N1CCN(c2ncc(C(F)(F)F)o2)CC1 ; CN(C(=O)COCCn1cc(C(F)(F)F)c2c(=O)[nH]ncc21)C1CCN(c2ncc(C(F)(F)F)cn2)CC1 ; CC(COCCC(=O)N1CCN(c2ncc(OC(F)(F)F)cn2)CC1)n1cc(C(F)(F)F)c2c(=O)[nH]ncc21]

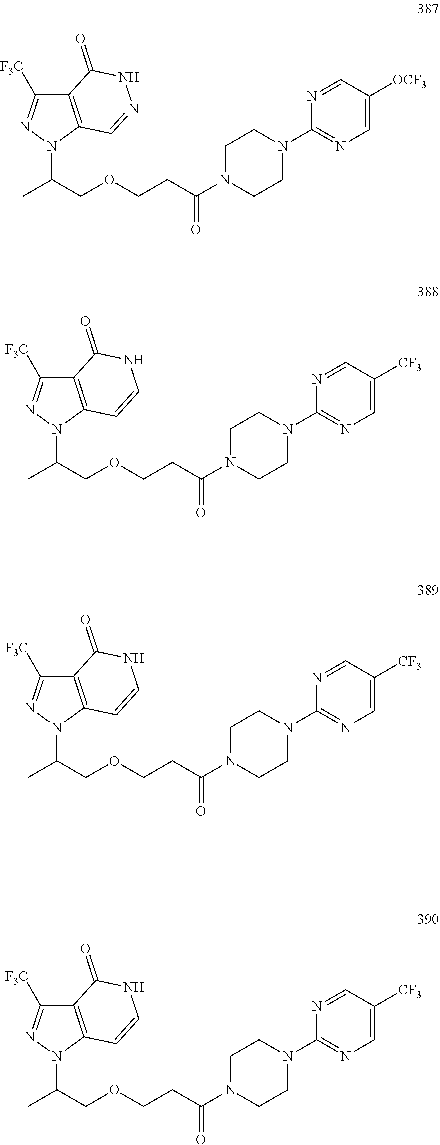 [SMILES: CC(COCCC(=O)N1CCN(c2ncc(OC(F)(F)F)cn2)CC1)n1nc(C(F)(F)F)c2c(=O)[nH]ncc21 ; CC(COCCC(=O)N1CCN(c2ncc(C(F)(F)F)cn2)CC1)n1nc(C(F)(F)F)c2c(=O)[nH]ccc21 ; CC(COCCC(=O)N1CCN(c2ncc(C(F)(F)F)cn2)CC1)n1nc(C(F)(F)F)c2c(=O)[nH]ccc21 ; CC(COCCC(=O)N1CCN(c2ncc(C(F)(F)F)cn2)CC1)n1nc(C(F)(F)F)c2c(=O)[nH]ccc21]

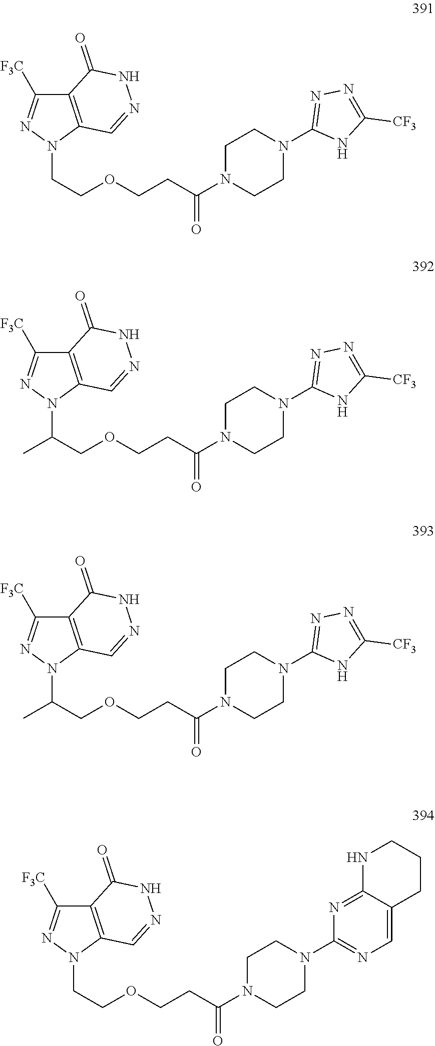 [SMILES: O=C(CCOCCn1nc(C(F)(F)F)c2c(=O)[nH]ncc21)N1CCN(c2nnc(C(F)(F)F)[nH]2)CC1 ; CC(COCCC(=O)N1CCN(c2nnc(C(F)(F)F)[nH]2)CC1)n1nc(C(F)(F)F)c2c(=O)[nH]ncc21 ; CC(COCCC(=O)N1CCN(c2nnc(C(F)(F)F)[nH]2)CC1)n1nc(C(F)(F)F)c2c(=O)[nH]ncc21 ; O=C(CCOCCn1nc(C(F)(F)F)c2c(=O)[nH]ncc21)N1CCN(c2ncc3c(n2)NCCC3)CC1]

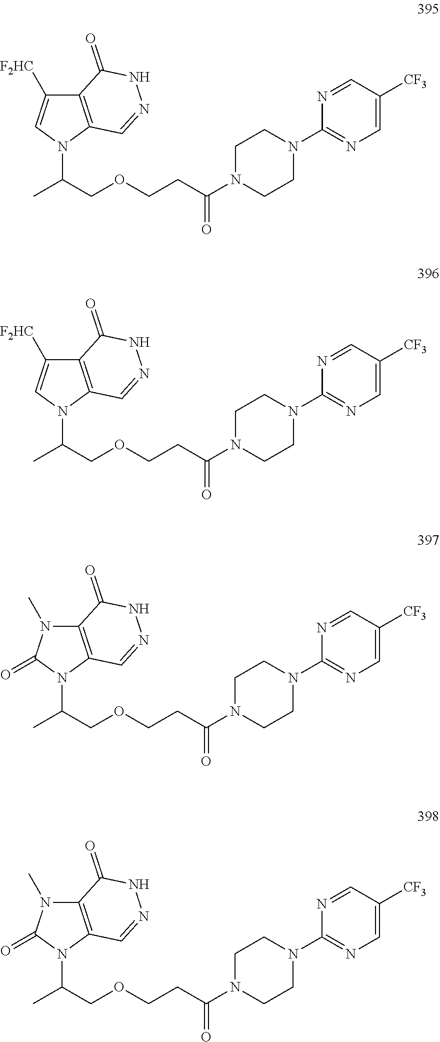 [SMILES: CC(COCCC(=O)N1CCN(c2ncc(C(F)(F)F)cn2)CC1)n1cc(C(F)F)c2c(=O)[nH]ncc21 ; CC(COCCC(=O)N1CCN(c2ncc(C(F)(F)F)cn2)CC1)n1cc(C(F)F)c2c(=O)[nH]ncc21 ; CC(COCCC(=O)N1CCN(c2ncc(C(F)(F)F)cn2)CC1)n1c(=O)n(C)c2c(=O)[nH]ncc21 ; CC(COCCC(=O)N1CCN(c2ncc(C(F)(F)F)cn2)CC1)n1c(=O)n(C)c2c(=O)[nH]ncc21]

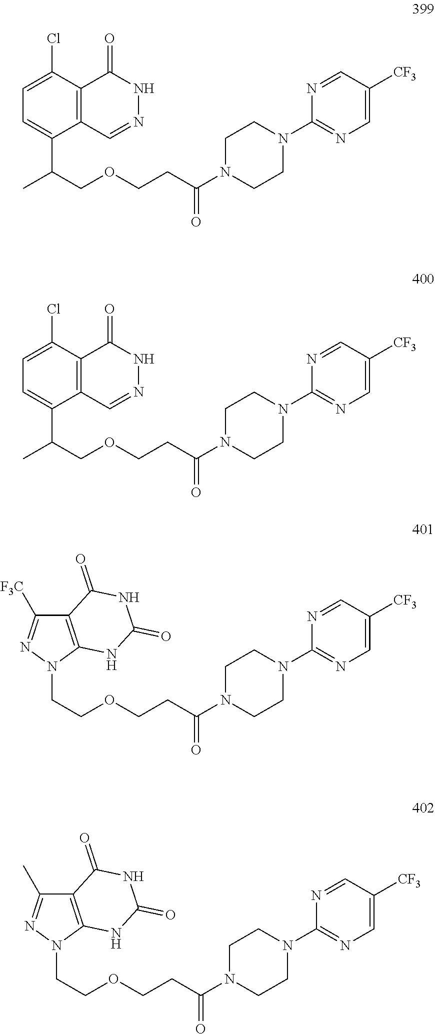 [SMILES: CC(COCCC(=O)N1CCN(c2ncc(C(F)(F)F)cn2)CC1)c1ccc(Cl)c2c(=O)[nH]ncc12 ; CC(COCCC(=O)N1CCN(c2ncc(C(F)(F)F)cn2)CC1)c1ccc(Cl)c2c(=O)[nH]ncc12 ; O=C(CCOCCn1nc(C(F)(F)F)c2c(=O)[nH]c(=O)[nH]c21)N1CCN(c2ncc(C(F)(F)F)cn2)CC1 ; Cc1nn(CCOCCC(=O)N2CCN(c3ncc(C(F)(F)F)cn3)CC2)c2[nH]c(=O)[nH]c(=O)c12]

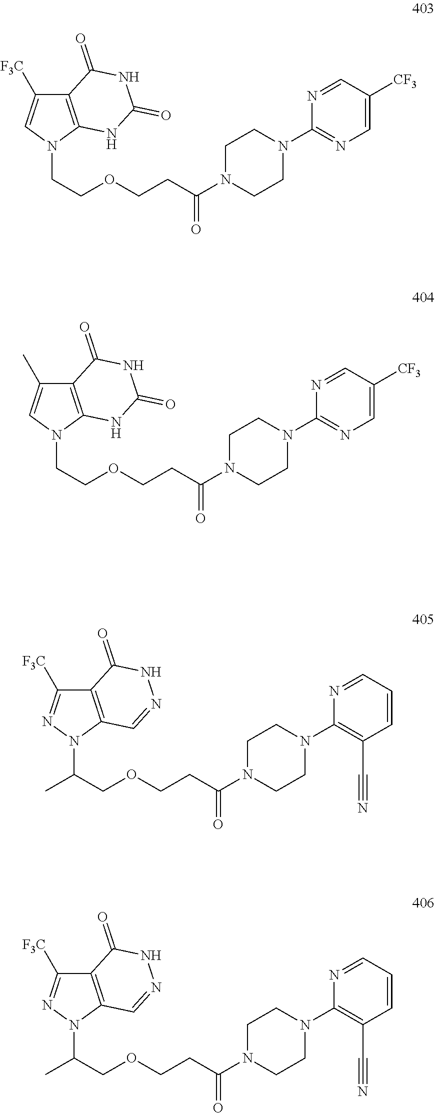 [SMILES: O=C(CCOCCn1cc(C(F)(F)F)c2c(=O)[nH]c(=O)[nH]c21)N1CCN(c2ncc(C(F)(F)F)cn2)CC1 ; Cc1cn(CCOCCC(=O)N2CCN(c3ncc(C(F)(F)F)cn3)CC2)c2[nH]c(=O)[nH]c(=O)c12 ; CC(COCCC(=O)N1CCN(c2ncccc2C#N)CC1)n1nc(C(F)(F)F)c2c(=O)[nH]ncc21 ; CC(COCCC(=O)N1CCN(c2ncccc2C#N)CC1)n1nc(C(F)(F)F)c2c(=O)[nH]ncc21]

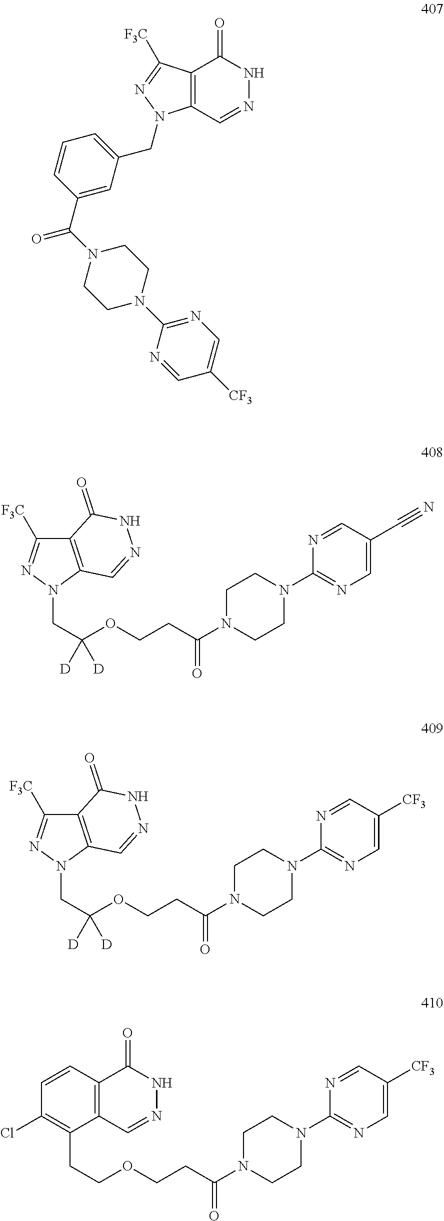 [SMILES: O=C(c1cccc(Cn2nc(C(F)(F)F)c3c(=O)[nH]ncc32)c1)N1CCN(c2ncc(C(F)(F)F)cn2)CC1 ; [2H]C([2H])(Cn1nc(C(F)(F)F)c2c(=O)[nH]ncc21)OCCC(=O)N1CCN(c2ncc(C#N)cn2)CC1 ; [2H]C([2H])(Cn1nc(C(F)(F)F)c2c(=O)[nH]ncc21)OCCC(=O)N1CCN(c2ncc(C(F)(F)F)cn2)CC1 ; O=C(CCOCCc1c(Cl)ccc2c(=O)[nH]ncc12)N1CCN(c2ncc(C(F)(F)F)cn2)CC1]

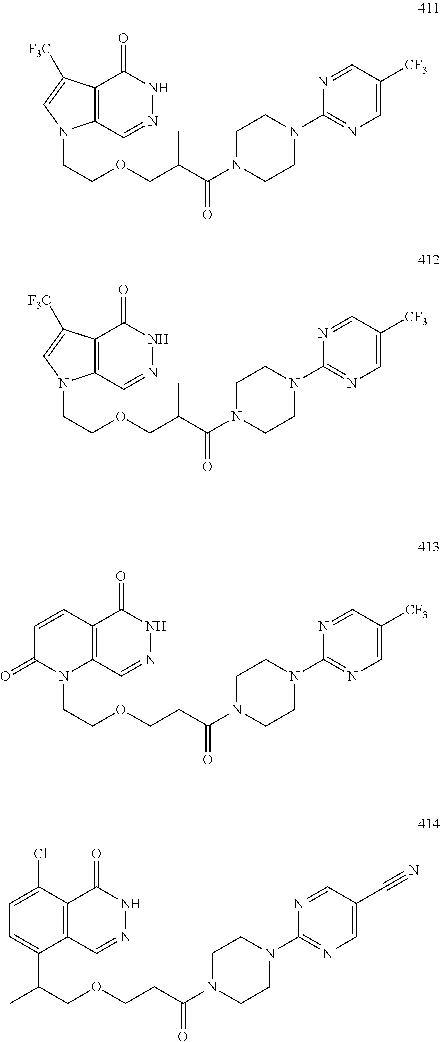 [SMILES: CC(COCCn1cc(C(F)(F)F)c2c(=O)[nH]ncc21)C(=O)N1CCN(c2ncc(C(F)(F)F)cn2)CC1 ; CC(COCCn1cc(C(F)(F)F)c2c(=O)[nH]ncc21)C(=O)N1CCN(c2ncc(C(F)(F)F)cn2)CC1 ; O=C(CCOCCn1c(=O)ccc2c(=O)[nH]ncc21)N1CCN(c2ncc(C(F)(F)F)cn2)CC1 ; CC(COCCC(=O)N1CCN(c2ncc(C#N)cn2)CC1)c1ccc(Cl)c2c(=O)[nH]ncc12]

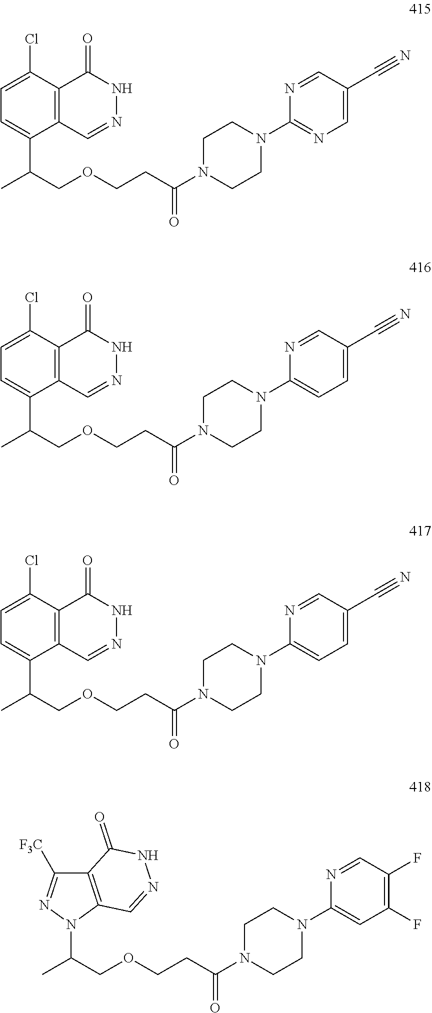 [SMILES: CC(COCCC(=O)N1CCN(c2ncc(C#N)cn2)CC1)c1ccc(Cl)c2c(=O)[nH]ncc12 ; CC(COCCC(=O)N1CCN(c2ccc(C#N)cn2)CC1)c1ccc(Cl)c2c(=O)[nH]ncc12 ; CC(COCCC(=O)N1CCN(c2ccc(C#N)cn2)CC1)c1ccc(Cl)c2c(=O)[nH]ncc12 ; CC(COCCC(=O)N1CCN(c2cc(F)c(F)cn2)CC1)n1nc(C(F)(F)F)c2c(=O)[nH]ncc21]

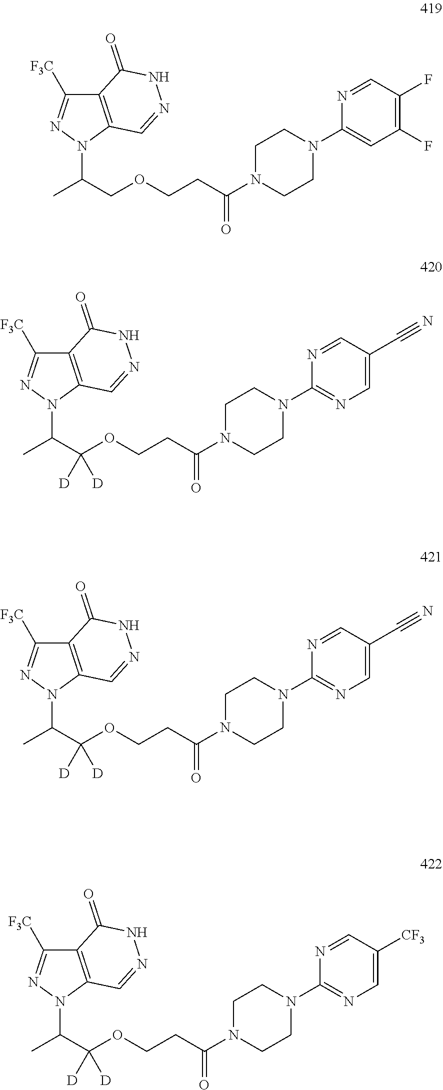 [SMILES: CC(COCCC(=O)N1CCN(c2cc(F)c(F)cn2)CC1)n1nc(C(F)(F)F)c2c(=O)[nH]ncc21 ; [2H]C([2H])(OCCC(=O)N1CCN(c2ncc(C#N)cn2)CC1)C(C)n1nc(C(F)(F)F)c2c(=O)[nH]ncc21 ; [2H]C([2H])(OCCC(=O)N1CCN(c2ncc(C#N)cn2)CC1)C(C)n1nc(C(F)(F)F)c2c(=O)[nH]ncc21 ; [2H]C([2H])(OCCC(=O)N1CCN(c2ncc(C(F)(F)F)cn2)CC1)C(C)n1nc(C(F)(F)F)c2c(=O)[nH]ncc21]

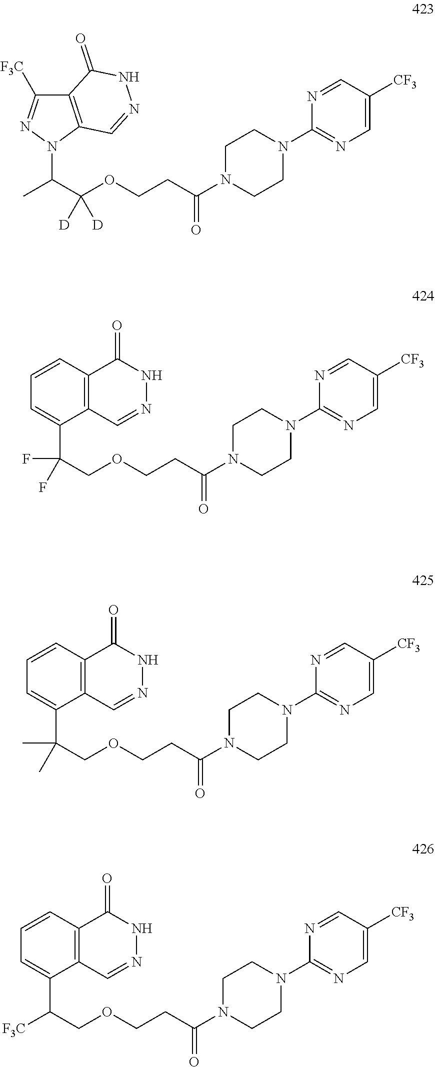 [SMILES: [2H]C([2H])(OCCC(=O)N1CCN(c2ncc(C(F)(F)F)cn2)CC1)C(C)n1nc(C(F)(F)F)c2c(=O)[nH]ncc21 ; O=C(CCOCC(F)(F)c1cccc2c(=O)[nH]ncc12)N1CCN(c2ncc(C(F)(F)F)cn2)CC1 ; CC(C)(COCCC(=O)N1CCN(c2ncc(C(F)(F)F)cn2)CC1)c1cccc2c(=O)[nH]ncc12 ; O=C(CCOCC(c1cccc2c(=O)[nH]ncc12)C(F)(F)F)N1CCN(c2ncc(C(F)(F)F)cn2)CC1]

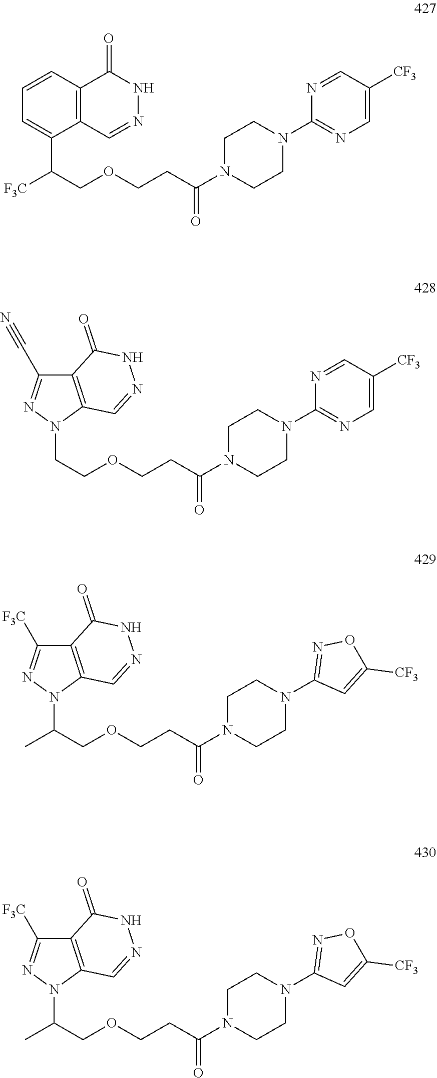 [SMILES: O=C(CCOCC(c1cccc2c(=O)[nH]ncc12)C(F)(F)F)N1CCN(c2ncc(C(F)(F)F)cn2)CC1 ; N#Cc1nn(CCOCCC(=O)N2CCN(c3ncc(C(F)(F)F)cn3)CC2)c2cn[nH]c(=O)c12 ; CC(COCCC(=O)N1CCN(c2cc(C(F)(F)F)on2)CC1)n1nc(C(F)(F)F)c2c(=O)[nH]ncc21 ; CC(COCCC(=O)N1CCN(c2cc(C(F)(F)F)on2)CC1)n1nc(C(F)(F)F)c2c(=O)[nH]ncc21]

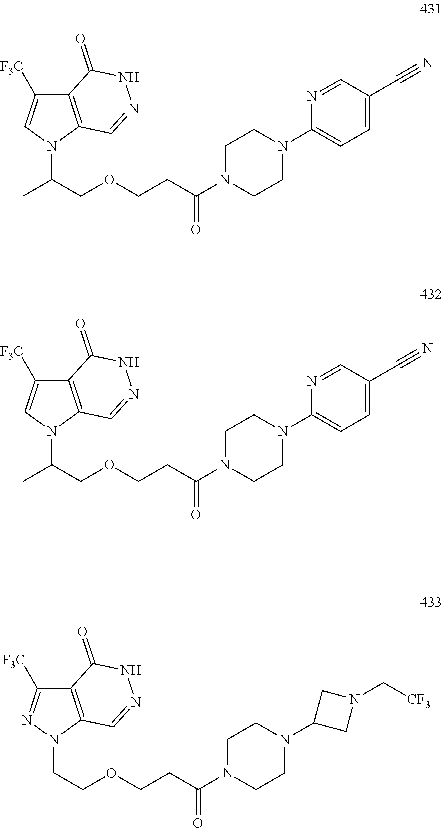 [SMILES: CC(COCCC(=O)N1CCN(c2ccc(C#N)cn2)CC1)n1cc(C(F)(F)F)c2c(=O)[nH]ncc21 ; CC(COCCC(=O)N1CCN(c2ccc(C#N)cn2)CC1)n1cc(C(F)(F)F)c2c(=O)[nH]ncc21 ; O=C(CCOCCn1nc(C(F)(F)F)c2c(=O)[nH]ncc21)N1CCN(C2CN(CC(F)(F)F)C2)CC1]

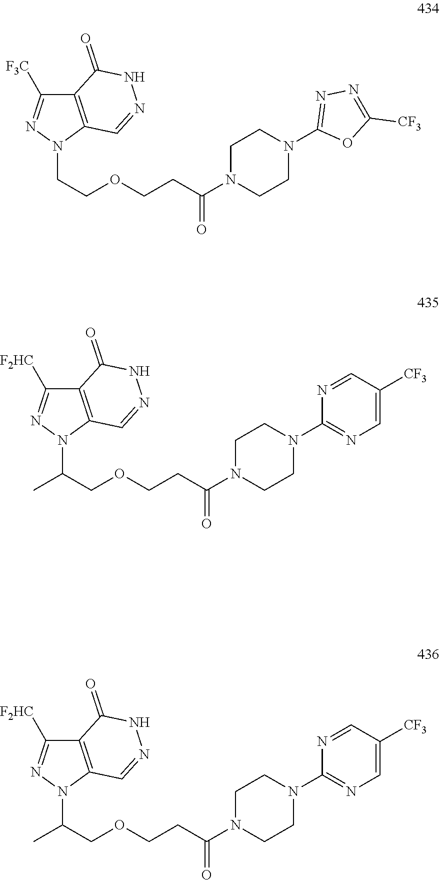 [SMILES: O=C(CCOCCn1nc(C(F)(F)F)c2c(=O)[nH]ncc21)N1CCN(c2nnc(C(F)(F)F)o2)CC1 ; CC(COCCC(=O)N1CCN(c2ncc(C(F)(F)F)cn2)CC1)n1nc(C(F)F)c2c(=O)[nH]ncc21 ; CC(COCCC(=O)N1CCN(c2ncc(C(F)(F)F)cn2)CC1)n1nc(C(F)F)c2c(=O)[nH]ncc21]

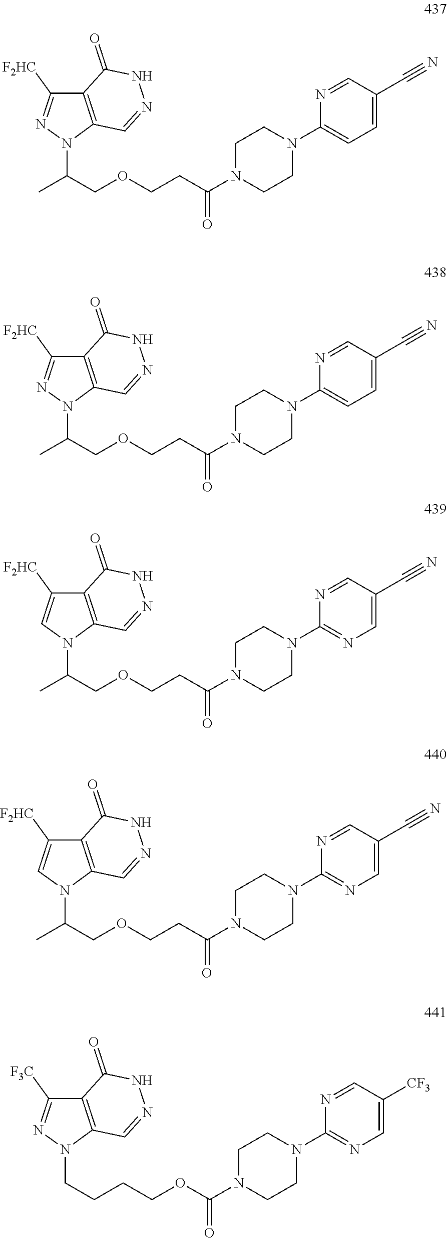 [SMILES: CC(COCCC(=O)N1CCN(c2ccc(C#N)cn2)CC1)n1nc(C(F)F)c2c(=O)[nH]ncc21 ; CC(COCCC(=O)N1CCN(c2ccc(C#N)cn2)CC1)n1nc(C(F)F)c2c(=O)[nH]ncc21 ; CC(COCCC(=O)N1CCN(c2ncc(C#N)cn2)CC1)n1cc(C(F)F)c2c(=O)[nH]ncc21 ; CC(COCCC(=O)N1CCN(c2ncc(C#N)cn2)CC1)n1cc(C(F)F)c2c(=O)[nH]ncc21 ; O=C(OCCCCn1nc(C(F)(F)F)c2c(=O)[nH]ncc21)N1CCN(c2ncc(C(F)(F)F)cn2)CC1]

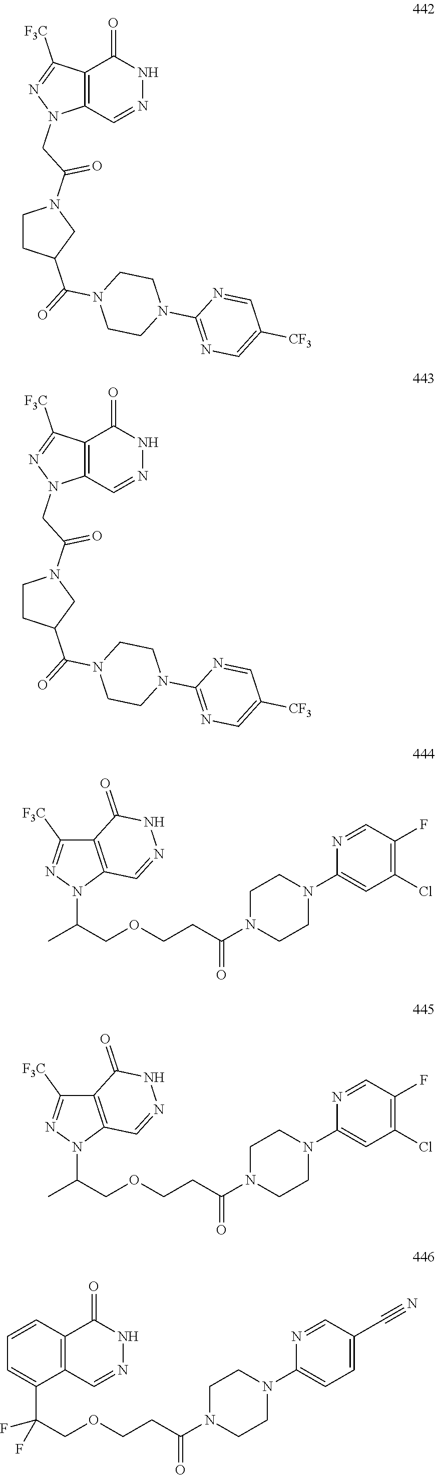 [SMILES: O=C(Cn1nc(C(F)(F)F)c2c(=O)[nH]ncc21)N1CCC(C(=O)N2CCN(c3ncc(C(F)(F)F)cn3)CC2)C1 ; O=C(Cn1nc(C(F)(F)F)c2c(=O)[nH]ncc21)N1CCC(C(=O)N2CCN(c3ncc(C(F)(F)F)cn3)CC2)C1 ; CC(COCCC(=O)N1CCN(c2cc(Cl)c(F)cn2)CC1)n1nc(C(F)(F)F)c2c(=O)[nH]ncc21 ; CC(COCCC(=O)N1CCN(c2cc(Cl)c(F)cn2)CC1)n1nc(C(F)(F)F)c2c(=O)[nH]ncc21 ; N#Cc1ccc(N2CCN(C(=O)CCOCC(F)(F)c3cccc4c(=O)[nH]ncc34)CC2)nc1]

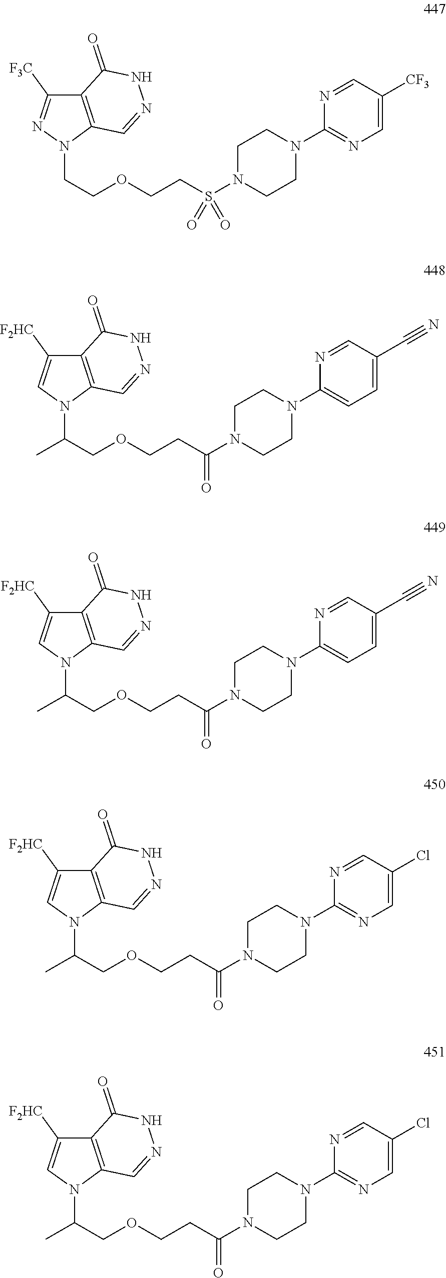 [SMILES: O=c1[nH]ncc2c1c(C(F)(F)F)nn2CCOCCS(=O)(=O)N1CCN(c2ncc(C(F)(F)F)cn2)CC1 ; CC(COCCC(=O)N1CCN(c2ccc(C#N)cn2)CC1)n1cc(C(F)F)c2c(=O)[nH]ncc21 ; CC(COCCC(=O)N1CCN(c2ccc(C#N)cn2)CC1)n1cc(C(F)F)c2c(=O)[nH]ncc21 ; CC(COCCC(=O)N1CCN(c2ncc(Cl)cn2)CC1)n1cc(C(F)F)c2c(=O)[nH]ncc21 ; CC(COCCC(=O)N1CCN(c2ncc(Cl)cn2)CC1)n1cc(C(F)F)c2c(=O)[nH]ncc21]

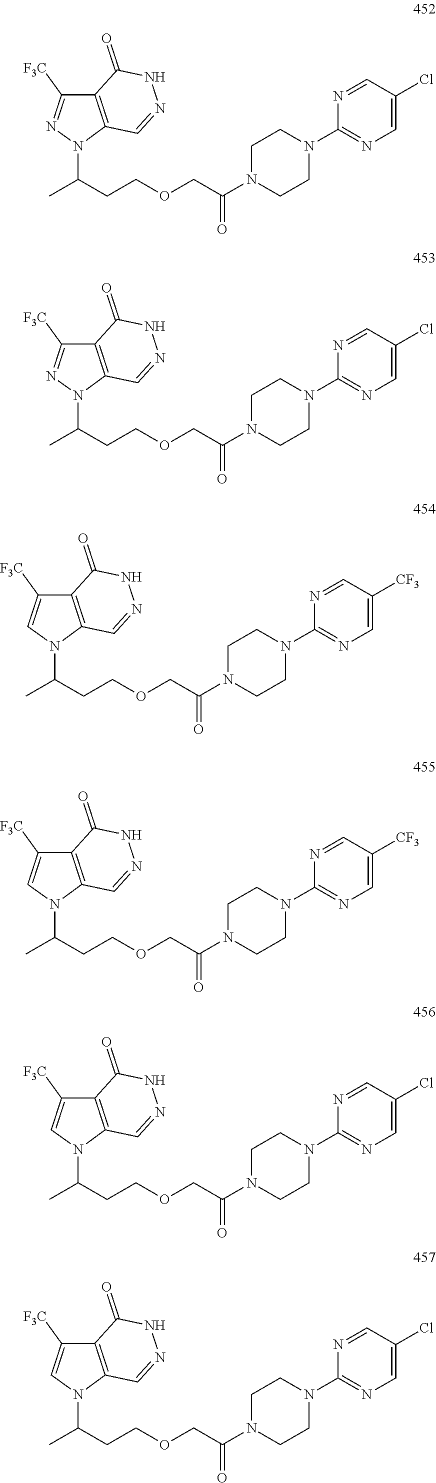 [SMILES: CC(CCOCC(=O)N1CCN(c2ncc(Cl)cn2)CC1)n1nc(C(F)(F)F)c2c(=O)[nH]ncc21 ; CC(CCOCC(=O)N1CCN(c2ncc(Cl)cn2)CC1)n1nc(C(F)(F)F)c2c(=O)[nH]ncc21 ; CC(CCOCC(=O)N1CCN(c2ncc(C(F)(F)F)cn2)CC1)n1cc(C(F)(F)F)c2c(=O)[nH]ncc21 ; CC(CCOCC(=O)N1CCN(c2ncc(C(F)(F)F)cn2)CC1)n1cc(C(F)(F)F)c2c(=O)[nH]ncc21 ; CC(CCOCC(=O)N1CCN(c2ncc(Cl)cn2)CC1)n1cc(C(F)(F)F)c2c(=O)[nH]ncc21 ; CC(CCOCC(=O)N1CCN(c2ncc(Cl)cn2)CC1)n1cc(C(F)(F)F)c2c(=O)[nH]ncc21]

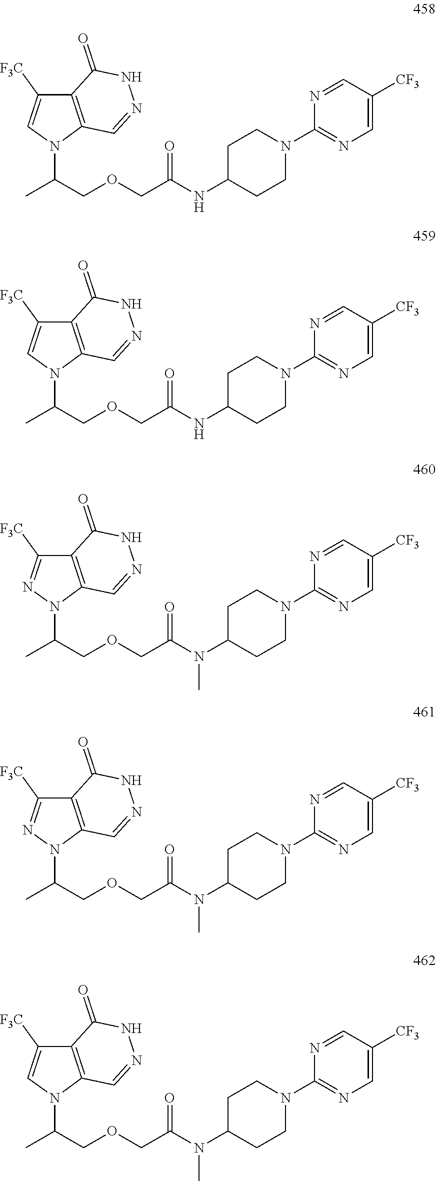 [SMILES: CC(COCC(=O)NC1CCN(c2ncc(C(F)(F)F)cn2)CC1)n1cc(C(F)(F)F)c2c(=O)[nH]ncc21 ; CC(COCC(=O)NC1CCN(c2ncc(C(F)(F)F)cn2)CC1)n1cc(C(F)(F)F)c2c(=O)[nH]ncc21 ; CC(COCC(=O)N(C)C1CCN(c2ncc(C(F)(F)F)cn2)CC1)n1nc(C(F)(F)F)c2c(=O)[nH]ncc21 ; CC(COCC(=O)N(C)C1CCN(c2ncc(C(F)(F)F)cn2)CC1)n1nc(C(F)(F)F)c2c(=O)[nH]ncc21 ; CC(COCC(=O)N(C)C1CCN(c2ncc(C(F)(F)F)cn2)CC1)n1cc(C(F)(F)F)c2c(=O)[nH]ncc21]

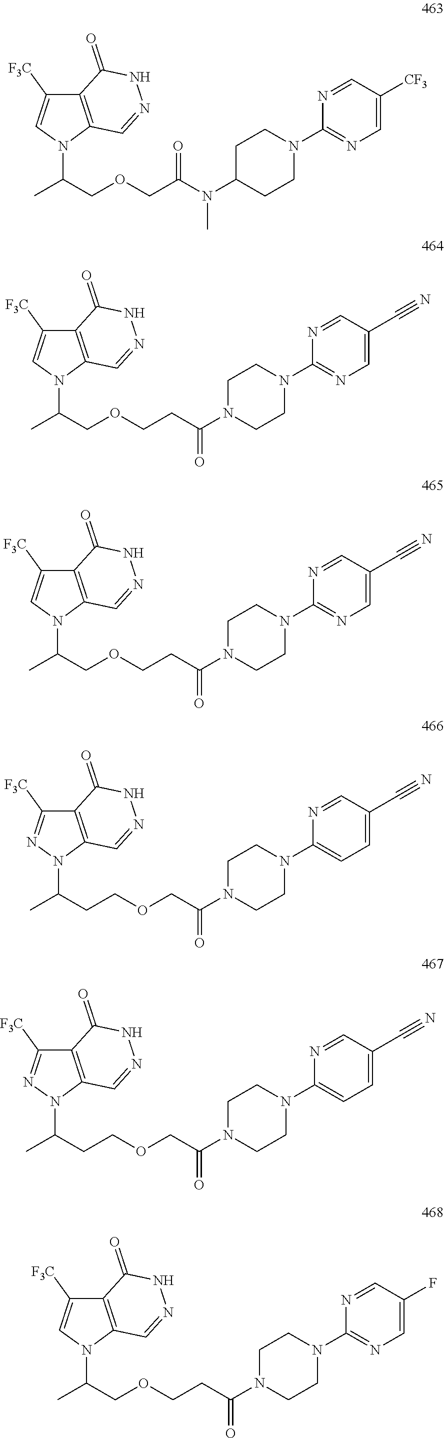 [SMILES: CC(COCC(=O)N(C)C1CCN(c2ncc(C(F)(F)F)cn2)CC1)n1cc(C(F)(F)F)c2c(=O)[nH]ncc21 ; CC(COCCC(=O)N1CCN(c2ncc(C#N)cn2)CC1)n1cc(C(F)(F)F)c2c(=O)[nH]ncc21 ; CC(COCCC(=O)N1CCN(c2ncc(C#N)cn2)CC1)n1cc(C(F)(F)F)c2c(=O)[nH]ncc21 ; CC(CCOCC(=O)N1CCN(c2ccc(C#N)cn2)CC1)n1nc(C(F)(F)F)c2c(=O)[nH]ncc21 ; CC(CCOCC(=O)N1CCN(c2ccc(C#N)cn2)CC1)n1nc(C(F)(F)F)c2c(=O)[nH]ncc21 ; CC(COCCC(=O)N1CCN(c2ncc(F)cn2)CC1)n1cc(C(F)(F)F)c2c(=O)[nH]ncc21]

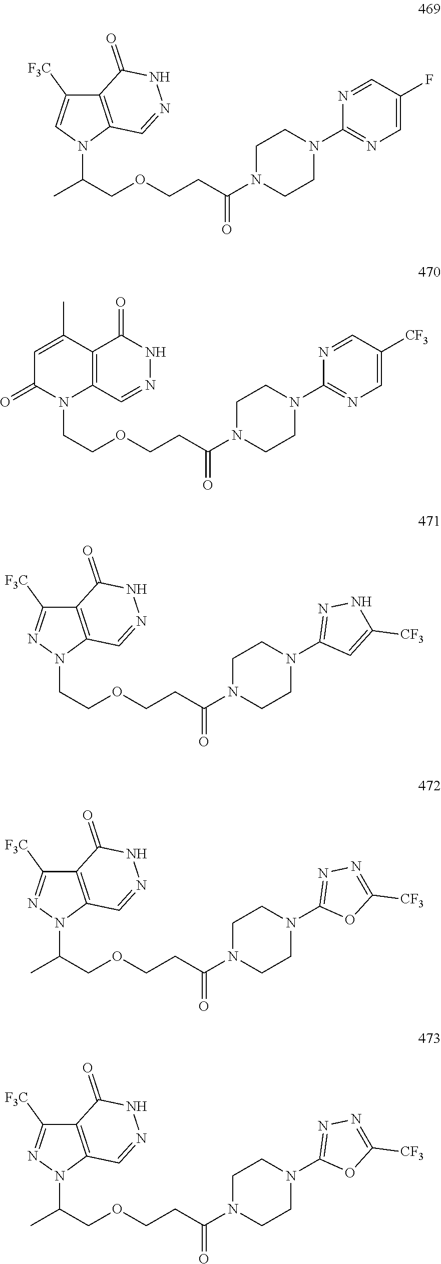 [SMILES: CC(COCCC(=O)N1CCN(c2ncc(F)cn2)CC1)n1cc(C(F)(F)F)c2c(=O)[nH]ncc21 ; Cc1cc(=O)n(CCOCCC(=O)N2CCN(c3ncc(C(F)(F)F)cn3)CC2)c2cn[nH]c(=O)c12 ; O=C(CCOCCn1nc(C(F)(F)F)c2c(=O)[nH]ncc21)N1CCN(c2cc(C(F)(F)F)[nH]n2)CC1 ; CC(COCCC(=O)N1CCN(c2nnc(C(F)(F)F)o2)CC1)n1nc(C(F)(F)F)c2c(=O)[nH]ncc21 ; CC(COCCC(=O)N1CCN(c2nnc(C(F)(F)F)o2)CC1)n1nc(C(F)(F)F)c2c(=O)[nH]ncc21]

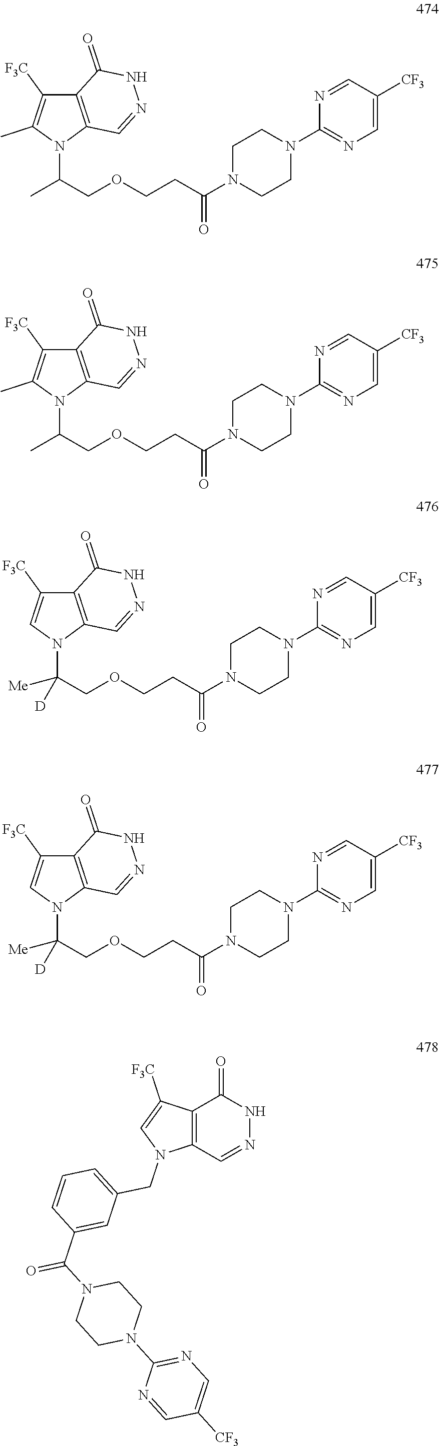 [SMILES: Cc1c(C(F)(F)F)c2c(=O)[nH]ncc2n1C(C)COCCC(=O)N1CCN(c2ncc(C(F)(F)F)cn2)CC1 ; Cc1c(C(F)(F)F)c2c(=O)[nH]ncc2n1C(C)COCCC(=O)N1CCN(c2ncc(C(F)(F)F)cn2)CC1 ; [2H]C(C)(COCCC(=O)N1CCN(c2ncc(C(F)(F)F)cn2)CC1)n1cc(C(F)(F)F)c2c(=O)[nH]ncc21 ; [2H]C(C)(COCCC(=O)N1CCN(c2ncc(C(F)(F)F)cn2)CC1)n1cc(C(F)(F)F)c2c(=O)[nH]ncc21 ; O=C(c1cccc(Cn2cc(C(F)(F)F)c3c(=O)[nH]ncc32)c1)N1CCN(c2ncc(C(F)(F)F)cn2)CC1]

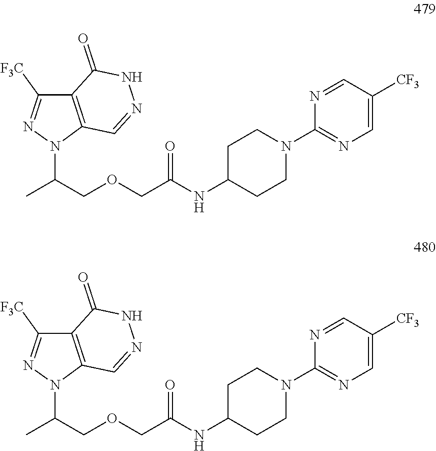 [SMILES: CC(COCC(=O)NC1CCN(c2ncc(C(F)(F)F)cn2)CC1)n1nc(C(F)(F)F)c2c(=O)[nH]ncc21 ; CC(COCC(=O)NC1CCN(c2ncc(C(F)(F)F)cn2)CC1)n1nc(C(F)(F)F)c2c(=O)[nH]ncc21]

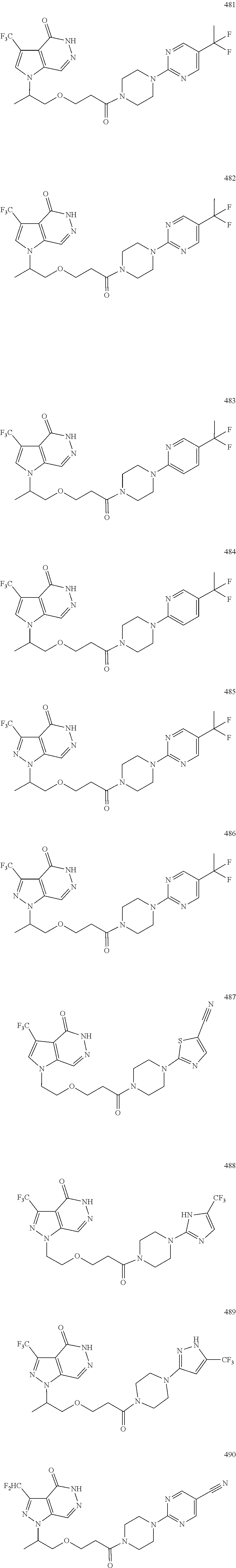

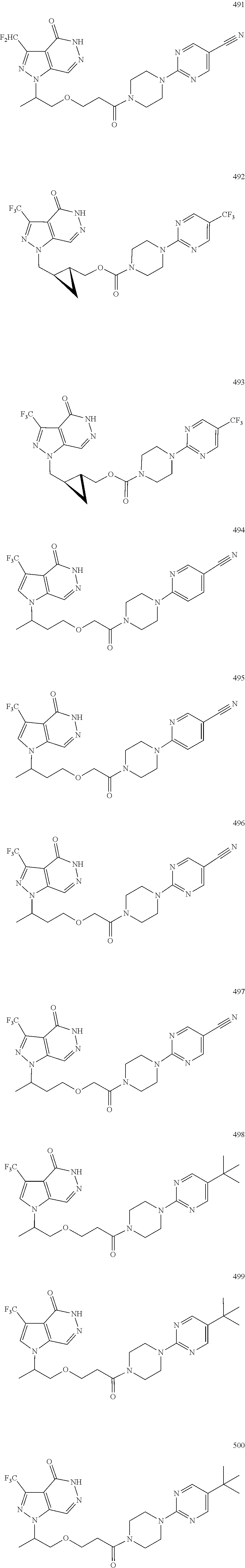

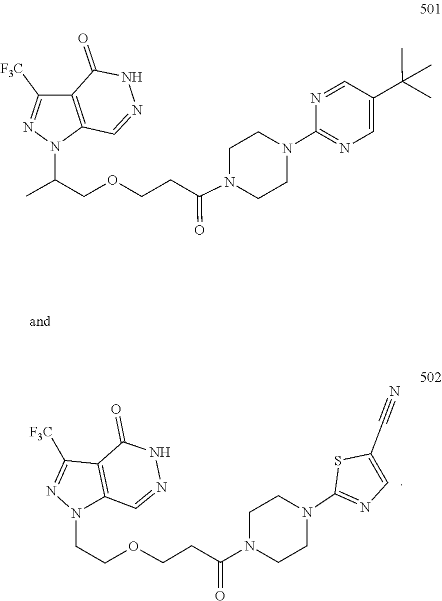

31. A compound according to claim 1, which compound comprises:
an isolated enantiomer, or
a mixture of two or more enantiomers, or
a mixture of two or more diastereomers, and/or epimers, or
a racemic mixture, or
a tautomer of the compound.

32-36. (canceled)

37. A pharmaceutical composition comprising a compound as defined in claim 1.

38. A pharmaceutical composition according to claim 37, further comprising a pharmaceutically acceptable additive and/or excipient, and/or wherein the compound is in the form of a pharmaceutically acceptable salt, hydrate, acid, ester, or other alternative form of the compound.

39. (canceled)

40. A pharmaceutical composition according to claim 37, further comprising a further agent for treating cancer; preferably wherein the further agent for treating cancer is selected from anti-microtubule agents, platinum coordination complexes, alkylating agents, antibiotic agents, topoisomerase II inhibitors, antimetabolites, topoisomerase I inhibitors, senolytic agents, hormones and hormone analogues, signal transduction pathway inhibitors, DNA damage repair pathway inhibitors, non-receptor tyrosine kinase angiogenesis inhibitors, immunotherapeutic agents (such as an anti-tumour vaccine, an oncolytic virus, an immune stimulatory antibody such as anti-CTLA4, anti-PD1, anti-PDL-1, anti-OX40, anti-41 BB, anti-CD27, anti-CD40, anti-LAG3, anti-TIM3, and anti-GITR, a novel adjuvant, a peptide, a cytokine, a chimeric antigen receptor T cell therapy (CAR-T), a small molecule immune modulator such as an IDO or TDO inhibitor or a pattern recognition receptor agonist such as a STING, TLR-9 or RIG-1 Helicase agonist, tumour microenvironment modulators, and anti-angiogenic agents), receptor tyrosine kinase inhibitors, cell growth inhibitors such as Ras and Raf inhibitors, proapoptotic agents and cell cycle signalling inhibitors.

41. A pharmaceutical composition according to claim 40, further comprising an agent selected from: an anti-tumour vaccine; a cancer immunotherapy treatment (such as an immune checkpoint modulator such as an anti-CTLA4, anti-PD1, anti PDL-1, anti-LAG3, or anti-TIM3 agent, and CD40, OX40, 41 BB or GITR agonists, IDO or TDO inhibitors); an immunomodulator such as a pattern recognition receptor agonist such as a STING, TLR-9 or RIG-1 Helicase; an immunosuppressant; a cytokine therapy; a tyrosine kinase inhibitor; and a chimeric antigen receptor T cell therapy (CAR-T).

42. A pharmaceutical kit for treating a cancer, which pharmaceutical kit comprises:
(a) a compound as defined in claim 1; and
(b) a further agent for treating cancer; preferably wherein the further agent for treating cancer is selected from anti-microtubule agents, platinum coordination complexes, alkylating agents, antibiotic agents, topoisomerase II inhibitors, antimetabolites, topoisomerase I inhibitors, senolytic agents, hormones and hormone analogues, signal transduction pathway inhibitors, DNA damage repair pathway inhibitors, non-receptor tyrosine kinase angiogenesis inhibitors, immunotherapeutic agents (such as an anti-tumour vaccine, an oncolytic virus, an immune stimulatory antibody such as anti-CTLA4, anti-PD1, anti-PDL-1, anti-OX40, anti-41 BB, anti-CD27, anti-CD40, anti-LAG3, anti-TIM3, and anti-GITR, a novel adjuvant, a peptide, a cytokine, a chimeric antigen receptor T cell therapy (CAR-T), a small molecule immune modulator such as a pattern recognition receptor agonist such as a STING, TLR-9 or RIG-1 Helicase agonist, tumour microenvironment modulators, and anti-angiogenic agents), receptor tyrosine kinase inhibitors, cell growth inhibitors such as Ras and Raf inhibitors, proapoptotic agents and cell cycle signalling inhibitors; wherein the compound and the further agent are suitable for administration simultaneously, sequentially or separately.

43. A method of treating a disease and/or a condition and/or a disorder, which method comprises administering to a patient a compound of claim 1.

44. A method according to claim 43, wherein the disease or condition or disorder is a disease, condition or disorder selected from: a cancer, an infectious disease, a central nervous system disease or disorder, and a pain condition.

45. A method according to claim 44 for treating a cancer, which method further comprises administering to the patient a further agent for treating a cancer; wherein the compound or composition and the further agent are administered simultaneously, sequentially or separately.

46. A method according to claim 43, wherein the patient is an animal, preferably a mammal, such as a human, canine or feline.

47. A method according to claim 46, wherein the patient is a human.

48. A method of synthesis of a compound claim 1, which method comprises conducting a reaction between (i) a first reactant comprising rings A and B bearing a portion of substituent group R¹ and (ii) a second reactant comprising the remainder of substituent group R¹ so as to form the PARP7 inhibitor compound.

49. A method according to claim 48, wherein the first reactant comprises a compound of general formula: 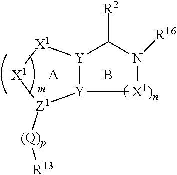
and the second reactant comprises a compound of general formula: 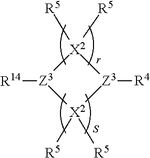
wherein R¹³ and R¹⁴ are each independently substituent groups which are removed during the reaction.

50. A method according to claim 48, wherein the reaction is carried out under conditions suitable for an amide formation, nucleophilic displacement or Michael addition reaction, optionally with one or more additional substitution steps.
